(19) United States
(12) Patent Application Publication
(10) Pub. No.: US 2022/0325363 A1
(43) Pub. Date: Oct. 13, 2022

(54) ASSAYS AND METHODS FOR DETECTION OF NUCLEIC ACIDS

(71) Applicant: Mammoth Biosciences, Inc., Brisbane, CA (US)

(72) Inventors: James Paul BROUGHTON, San Francisco, CA (US); Jasmeet Singh, Santa Clara, CA (US); Clare Louise FASCHING, Redwood City, CA (US); Maria-Nefeli TSALOGLOU, San Francisco, CA (US); Pedro Patrick Draper GALARZA, Oakland, CA (US); Janice Sha CHEN, San Francisco, CA (US); Xin MIAO, Mountain View, CA (US); Lucas HARRINGTON, San Francisco, CA (US); Daniel Thomas DRZAL, Redwood City, CA (US); Sarah Jane Shapiro, Pacifica, CA (US)

(21) Appl. No.: 17/555,236

(22) Filed: Dec. 17, 2021

Related U.S. Application Data
(63) Continuation of application No. PCT/US2020/038242, filed on Jun. 17, 2020.
(60) Provisional application No. 62/985,850, filed on Mar. 5, 2020.
(60) Provisional application No. 62/944,926, filed on Dec. 6, 2019.
(60) Provisional application No. 62/881,809, filed on Aug. 1, 2019.
(60) Provisional application No. 62/879,325, filed on Jul. 26, 2019.
(60) Provisional application No. 62/863,178, filed on Jun. 18, 2019.

(51) Int. Cl. C12Q 1/70; B01L 3/00

(52) U.S. Cl. CPC C12Q 1/701; B01L 3/5023; B01L 2300/069; B01L 2200/16; B01L 2300/0883; B01L 2400/0622; B01L 2400/086; B01L 2300/1805; C12Q 1/6823

(57) ABSTRACT

Described herein are devices, systems, fluidic devices, kits, and methods for detection of target nucleic acids.

### CROSS-REFERENCE

This application is a continuation of International Application No. PCT/US2020/038242, filed Jun. 17, 2020, which claims priority to and benefit from U.S. Provisional Application No.: 62/863,178, filed on June 18, 2019, U.S. Provisional Application No.: 62/879,325, filed on Jul. 26, 2019, U.S. Provisional Application No.: 62/881,809, filed on Aug. 1, 2019, U.S. Provisional Application No.: 62/944,926, filed on Dec. 6, 2019, and U.S. Provisional Application No.: 62/985,850, filed on Mar. 5, 2020, the entire contents of each of which are herein incorporated by reference.

### SEQUENCE LISTING

The instant application contains a Sequence Listing which has been submitted electronically in ASCII format and is hereby incorporated by reference in its entirety. Said ASCII copy, created on Jul. 22, 2020, is named 53694-730_601_SL.txt and is 1,121,102 bytes in size.

### BACKGROUND

Various communicable diseases can easily spread from an individual or environment to an individual. These diseases may include but are not limited to influenza. Individuals with influenza may have poor outcomes. The detection of the ailments, especially at the early stages of infection, may provide guidance on treatment or intervention to reduce the progression or transmission of the ailment.

### SUMMARY

In various aspects, the present disclosure provides a microfluidic cartridge for detecting a target nucleic acid comprising: an amplification chamber fluidically connected to a valve; a detection chamber fluidically connected to the valve, wherein the valve is connected to a sample metering channel; a detection reagent chamber fluidically connected to the detection chamber via a resistance channel, the detection reagent chamber comprising a programmable nuclease, a guide nucleic acid, and a labeled detector nucleic acid, wherein the labeled detector nucleic acid is capable of being cleaved upon binding of the guide nucleic acid to a segment of a target nucleic acid.

In some aspects, the sample metering channel controls volumes of liquids dispensed in a channel or chamber. In some aspects, the sample metering channel is fluidically connected to the detection chamber. In some aspects, the resistance channel has a serpentine path, an angular path, or a circuitous path. In some aspects, the valve is a rotary valve, pneumatic valve, a hydraulic valve, an elastomeric valve. In some aspects, the resistance channel is fluidically connected with the valve. In some aspects, the valve comprises casing comprising a “substrate” or an “over-mold.” In some aspects, the valve is actuated by a solenoid. In some aspects, the valve is controlled manually, magnetically, electrically, thermally, by a bistable circuit, with a piezoelectric material, electrochemically, with phase change, rheologically, pneumatically, with a check valve, with capillarity, or any combination thereof. In some aspects, the rotary valve fluidically connects at least 3, at least, 4, or at least 5 chambers.

In some aspects, the microfluidic cartridge further comprises an amplification reagent chamber fluidically connected to the amplification chamber. In some aspects, the microfluidic cartridge further comprises a sample chamber fluidically connected to the amplification reagent chamber. In some aspects, the microfluidic cartridge further comprises a sample inlet connected to the sample chamber. In some aspects, the sample inlet is sealable. In some aspects, the sample inlet forms a seal around the sample.

In some aspects, the sample chamber comprises a lysis buffer. In some aspects, the microfluidic cartridge further comprises a lysis buffer storage chamber fluidically connected to the sample chamber. In some aspects, the lysis buffer storage chamber comprises a lysis buffer. In some aspects, the lysis buffer is a dual lysis/amplification buffer.

In some aspects, the lysis buffer storage chamber is fluidically connected to the sample chamber through a second valve. In some aspects, the sample chamber is fluidically connected to the amplification chamber through the amplification reagent chamber. In some aspects, the sample chamber is fluidically connected to the amplification reagent chamber through the amplification chamber. In some aspects, the microfluidic cartridge is configured to direct fluid bidirectionally between the amplification reagent chamber and amplification chamber. In some aspects, the detection reagent chamber is fluidically connected to the amplification chamber. In some aspects, the amplification chamber is fluidically connected to the detection chamber through the detection reagent chamber. In some aspects, comprising a reagent port above the detection chamber configured to deliver fluid from the detection reagent chamber to the detection chamber. In some aspects, the amplification chamber is fluidically connected to the detection reagent chamber through the detection chamber.

In some aspects, the resistance channel is configured to reduce backflow into the detection chamber and the detection reagent chamber. In some aspects, the sample metering channel is configured to direct a predetermined volume of fluid from the detection reagent chamber to the detection chamber. In some aspects, the amplification chamber and detection chamber are thermally isolated. In some aspects, the detection reagent chamber is fluidically connected to the detection chamber. In some aspects, the detection reagent chamber is fluidically connected to the detection chamber via a second resistance channel. In some aspects, the resistance channel or the second resistance channel is a serpentine resistance channel. In some aspects, the resistance channel or the second resistance channel comprises at least two hairpins. In some aspects, the resistance channel or the second resistance channel comprises at least one, at least 2, at least 3, or at least 4 right angles.

In some aspects, the amplification chamber comprises a sealable sample inlet. In some aspects, the sample inlet is configured to form a seal around a swab. In some aspects, microfluidic cartridge is configured to connect to a first pump to pump fluid from the amplification chamber to the detection chamber. In some aspects, microfluidic cartridge is configured to connect to a second pump to pump fluid from the detection reagent chamber to the detection chamber. In some aspects, first pump or the second pump is a pneumatic pump, a peristaltic pump, a hydraulic pump, or a syringe pump. In some aspects, the amplification chamber is fluidically connected to a port configured to receive pneumatic pressure. In some aspects, the amplification chamber is fluidically connected to the port through a channel. In some aspects, the amplification reagent chamber is connected to a second port configured to receive pneumatic pressure. In some aspects, the amplification reagent chamber is fluidically connected to the second port through a second channel.

In some aspects, the microfluidic cartridge is configured to connect to a third pump to pump fluid from the amplification reagent chamber to the amplification chamber. In some aspects, the third pump is a pneumatic pump, a peristaltic pump, a hydraulic pump, or a syringe pump. In some aspects, the detection reagent chamber is connected to a port configured to receive pneumatic pressure. In some aspects, the detection reagent chamber is fluidically connected to a third port through a third channel.

In some aspects, the microfluidic cartridge is configured to connect to a fourth pump to pump fluid from the detection reagent chamber to the detection chamber. In some aspects, the fourth pump is a pneumatic pump, a peristaltic pump, a hydraulic pump, or a syringe pump.

In some aspects, the microfluidic cartridge further comprises a plurality of ports configured to couple to a gas manifold, wherein the plurality of ports is configured to receive pneumatic pressure. In some aspects, any chamber of the microfluidic cartridge is connected to the plurality of ports. In some aspects, the valve is opened upon application of current electrical signal.

In some aspects, the detection reagent chamber is circular. In some aspects, the detection reagent chamber is elongated. In some aspects, the detection reagent chamber is hexagonal. In some aspects, a region of the resistance channel is molded to direct flow in a direction perpendicular to the net flow direction. In some aspects, a region of the resistance channel is molded to direct flow in a direction perpendicular to the axis defined by two ends of the resistance channel. In some aspects, a region of the resistance channel is molded to direct flow along the z-axis of the microfluidic cartridge. In some aspects, the valve is fluidically connected to two detection chambers via an amplification mix splitter. In some aspects, the valve is fluidically connected to 3, 4, 5, 6, 7, 8, 9, or 10 detection chambers via an amplification mix splitter.

In some aspects, the microfluidic cartridge further comprises a second valve fluidically connected to the detection reagent chamber and the detection chamber. In some aspects, the detection chamber is vented with a hydrophobic PTFE vent. In some aspects, the detection chamber comprises an optically transparent surface.

In some aspects, the amplification chamber is configured to hold from 10 μL to 500 μL of fluid. In some aspects, the amplification reagent chamber is configured to hold from 10 μL to 500 μL of fluid. In some aspects, the microfluidic cartridge is configured to accept from 2 μL to 100 μL of a sample comprising a nucleic acid. In some aspects, the amplification reagent chamber comprises between 5 and 200 μl an amplification buffer. In some aspects, the amplification chamber comprises 45 μ1 amplification buffer. In some aspects, the detection reagent chamber stores from 5 to 200 μl of fluid containing the programmable nuclease, the guide nucleic acid, and the labeled detector nucleic acid.

In some aspects, the microfluidic cartridge comprises 2, 3, 4, 5, 6, 7, or 8 detection chambers. In some aspects, the 2, 3, 4, 5, 6, 7, or 8 detection chambers are fluidically connected to a single sample chamber. In some aspects, the detection chamber holds up to 100 μL, 200 μL, 300 μL, or 400 μL of fluid.

In some aspects, the microfluidic cartridge comprises 5-7 layers. In some aspects, the microfluidic cartridge comprises layers as shown in FIG. 130B. In some aspects, the microfluidic cartridge further comprises a sample inlet configured to adapt with a slip luer tip. In some aspects, the slip luer tip is adapted to fit a syringe holding a sample. In some aspects, the sample inlet is capable of being hermetically sealed.

In some aspects, the microfluidic cartridge further comprises a sliding valve. In some aspects, the sliding valve connects the amplification reagent chamber to the amplification chamber. In some aspects, the sliding valve connects the amplification chamber to the detection reagent chamber. In some aspects, the sliding valve connects the amplification reagent chamber to the detection chamber.

In various aspects, the present disclosure provides a manifold configured to accept the microfluidic cartridge. In some aspects, the manifold comprises a pump configured to pump fluid into the detection chamber, an illumination source configured to illuminate the detection chamber, a detector configured to detect a detectable signal produced by the labeled detector nucleic acid, and a heater configured to heat the amplification chamber. In some aspects, the manifold further comprises a second heater configured to heat the detection chamber.

In some aspects, the illumination source is a broad spectrum light source. In some aspects, the illumination source light produces an illumination with a bandwidth of less than 5 nm. In some aspects, the illumination source is a light emitting diode. In some aspects, the light emitting diode produces white light, blue light, or green light.

In some aspects, the detectable signal is light. In some aspects, the detector is a camera or a photodiode. In some aspects, the detector has a detection bandwidth of less than 100 nm, less than 75 nm, less than 50 nm, less than 40 nm, less than 30 nm, less than 20 nm, less than 10 nm, or less than 5 nm.

In some aspects, the manifold further comprises an optical filter configured to be between the detection chamber and the detector. In some aspects, the amplification chamber comprises amplification reagents. In some aspects, the amplification reagent chamber comprises amplification reagents. In some aspects, the amplification reagents comprise a primer, a polymerase, dNTPs, an amplification buffer. In some aspects, the amplification chamber comprises a lysis buffer. In some aspects, the amplification reagent chamber comprises a lysis buffer. In some aspects, the amplification reagents comprise a reverse transcriptase. In some aspects, the amplification reagents comprise reagents for thermal cycling amplification. In some aspects, the amplification reagents comprise reagents for isothermal amplification. In some aspects, the amplification reagents comprise reagents for transcription mediated amplification (TMA), helicase dependent amplification (HDA), circular helicase dependent amplification (cHDA), strand displacement amplification (SDA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). In some aspects, the amplification reagents comprise reagents for loop mediated amplification (LAMP).

In some aspects, the lysis buffer and the amplification buffer are a single buffer. In some aspects, the lysis buffer storage chamber comprises a lysis buffer. In some aspects, the lysis buffer has a pH of from pH 4 to pH 5.

In some aspects, the microfluidic cartridge further comprises reverse transcription reagents. In some aspects, the reverse transcription reagents comprise a reverse transcriptase, a primer, and dNTPs. In some aspects, the programmable nuclease comprises an RuvC catalytic domain. In some aspects, the programmable nuclease is a type V CRISPR/Cas effector protein. In some aspects, the type V CRISPR/Cas effector protein is a Cas12 protein. In some aspects, the Cas12 protein comprises a Cas12a polypeptide, a Cas12b polypeptide, a Cas12c polypeptide, a Cas12d polypeptide, a Cas12e polypeptide, a C2c4 polypeptide, a C2c8 polypeptide, a C2c5 polypeptide, a C2c10 polypeptide, and a C2c9 polypeptide. In some aspects, the Cas12 protein has at least 80%, at least 85%, at least 90%, at least 92%, at least 95%, at least 97%, or at least 99% sequence identity to any one of SEQ ID NO: 27-SEQ ID NO: 37. In some aspects, the Cas12 protein is selected from SEQ ID NO: 27-SEQ ID NO: 37.

In some aspects, the type V CRIPSR/Cas effector protein is a Cas14 protein. In some aspects, the Cas14 protein comprises a Cas14a polypeptide, a Cas14b polypeptide, a Cas14c polypeptide, a Cas14d polypeptide, a Cas14e polypeptide, a Cas 14f polypeptide, a Cas14g polypeptide, a Cas14h polypeptide, a Cas14i polypeptide, a Cas14j polypeptide, or a Cas14k polypeptide. In some aspects, the Cas14 protein has at least 80%, at least 85%, at least 90%, at least 92%, at least 95%, at least 97%, or at least 99% sequence identity to any one of SEQ ID NO: 38-SEQ ID NO: 129. In some aspects, the Cas14 protein is selected from SEQ ID NO: 38-SEQ ID NO: 129.

In some aspects, the type V CRIPSR/Cas effector protein is a CasΦ protein. In some aspects, the CasΦ protein has at least 80%, at least 85%, at least 90%, at least 92%, at least 95%, at least 97%, or at least 99% sequence identity to any one of SEQ ID NO: 274-SEQ ID NO: 321. In some aspects, the Case protein is selected from SEQ ID NO: 274-SEQ ID NO: 321.

In some aspects, microfluidic cartridge further provides one or more chambers for in vitro transcribing amplified coronavirus target nucleic acid. In some aspects, the in vitro transcribing comprises contacting the amplified coronavirus target nucleic acid to reagents for in vitro transcription. In some aspects, the reagents for in vitro transcription comprise an RNA polymerase, NTPs, and a primer.

In some aspects, the programable nuclease comprises a HEPN cleaving domain. In some aspects, the programmable nuclease is a type VI CRISPR/Cas effector protein. In some aspects, the type VI CRISPR/Cas effector protein is a Cas13 protein. In some aspects, the Cas13 protein comprises a Cas13a polypeptide, a Cas13b polypeptide, a Cas13c polypeptide, a Cas13c polypeptide, a Cas13d polypeptide, or a Cas13e polypeptide. In some aspects, the Cas13 protein has at least 80%, at least 85%, at least 90%, at least 92%, at least 95%, at least 97%, or at least 99% sequence identity to any one of SEQ ID NOs: 130-SEQ ID NO: 137. In some aspects, the Cas13 protein is selected from SEQ ID NOs: 130-SEQ ID NO: 137.

In some aspects, the target nucleic acid is from a virus. In some aspects, the virus comprises a respiratory virus. In some aspects, the respiratory virus is an upper respiratory virus. In some aspects, the virus comprises an influenza virus. In some aspects, the virus comprises a coronavirus.

In some aspects, the coronavirus target nucleic acid is from SARS-CoV-2. In some aspects, the coronavirus target nucleic acid is from an N gene, an E gene, or a combination thereof. In some aspects, the coronavirus target nucleic acid has a sequence of any one of SEQ ID NO: 333-SEQ ID NO: 338. In some aspects, the influenza virus comprises an influenza A virus, influenza B virus, or a combination thereof. In some aspects, the plurality of target sequences comprises sequences from influenza A virus, influenza B virus, and a third pathogen.

In some aspects, the guide nucleic acid is a guide RNA. In some aspects, the guide nucleic acid has at least 80%, at least 85%, at least 90%, at least 92%, at least 95%, at least 97%, or at least 99% sequence identify to any one of SEQ ID NO: 323-SEQ ID NO: 328. In some aspects, the guide nucleic acid is selected from any one of SEQ ID NO: 323-SEQ ID NO: 328. In some aspects, the microfluidic cartridge comprises a control nucleic acid. In some aspects, the control nucleic acid is in the detection chamber. In some aspects, the control nucleic acid is RNaseP. In some aspects, the control nucleic acid has a sequence of SEQ ID NO: 379.

In some aspects, the guide nucleic acid has at least 80%, at least 85%, at least 90%, at least 92%, at least 95%, at least 97%, or at least 99% sequence identify to any one of SEQ ID NO: 330-SEQ ID NO: 332. In some aspects, the guide nucleic acid is selected from any one of SEQ ID NO: 330-SEQ ID NO: 332. In some aspects, the guide nucleic acid targets a plurality of target sequences.

In some aspects, the microfluidic cartridge comprises a plurality of guide sequences tiled against a virus. In some aspects, the labeled detector nucleic acid comprises a single stranded reporter comprising a detection moiety. In some aspects, the detection moiety is a fluorophore, a FRET pair, a fluorophore/quencher pair, or an electrochemical reporter molecule. In some aspects, the electrochemical reporter molecule comprises a species shown in FIG. 149. In some aspects, the labeled detector produced a detectable signal upon cleavage of the detector nucleic acid. In some aspects, the detectable signal is a colorimetric signal, a fluorescence signal, an amperometric signal, or a potentiometric signal.

In various aspects, the present disclosure provides a method of detecting a target nucleic acid, the method comprising: providing a sample from a subject; adding the sample to a microfluidic cartridge; correlating a detectable signal to the presence or absence of a target nucleic acid; and optionally quantifying the detectable signal, thereby quantifying an amount of the target nucleic acid present in the sample.

In some aspects, a microfluidic cartridge may be used in a method for detecting a target nucleic acid. In some aspects, a system may be used in a method for detecting a targeting nucleic acid. In some aspects, a programmable nuclease may be used in a method for detecting a target nucleic acid. In some aspects, a composition may be used in a method for detecting a target a nucleic acid. In some aspects, a DNA-activated programmable RNA nuclease may be used in a method for assaying for a target deoxyribonucleic acid from a virus in a sample. In some aspects, a DNA-activated programmable RNA nuclease may be used in a method of assaying for a target ribonucleic acid from a virus in a sample. In some aspects, a programmable nuclease may be used in a method for detecting a target nucleic acid in a sample.

In various aspects, the present disclosure provides a system for detecting a target nucleic acid, said system comprising: a guide nucleic acid targeting a target sequence from a virus; a programmable nuclease capable of being activated when complexed with the guide nucleic acid and the target sequence; and a reporter, wherein the reporter is capable of being cleaved by the activated nuclease, thereby generating a detectable signal.

In some aspects, the reporter comprises a single stranded reporter comprising a detection moiety. In some aspects, the virus comprises an influenza virus. In some aspects, the influenza virus comprises an influenza A virus, influenza B virus, or a combination thereof. In some aspects, the virus comprises a respiratory virus. In some aspects, the respiratory virus is an upper respiratory virus. In some aspects, the guide nucleic acid targets a plurality of target sequences.

In some aspects, the system comprises a plurality of guide sequences tiled against the virus. In some aspects, the plurality of target sequences comprises sequences from influenza A virus, influenza B virus, and a third pathogen. In some aspects, the single stranded reporter comprises the detection moiety at the 5′ end. In some aspects, the single stranded reporter comprises a biotin-dT/FAM moiety or a biotin-dT/ROX moiety. In some aspects, the single stranded reporter comprises a chemical functional handle at the 3′end capable of being conjugated to a substrate.

In some aspects, the substrate is a magnetic bead. In some aspects, the substrate is a surface of a reaction chamber. In some aspects, downstream of the reaction chamber is a test line. In some aspects, the test line comprises a streptavidin. In some aspects, downstream of the test line is a flow control line. In some aspects, the flow control line comprises an anti-IgG antibody. In some aspects, the anti-IgG antibody comprises an anti-rabbit IgG antibody.

In some aspects, the activated nuclease is capable of cleaving the single stranded reporter and releases the biotin-dT/FAM moiety or the biotin-dT/ROX moiety. In some aspects, the biotin-dT/FAM moiety is capable of binding the streptavidin at the test line. In some aspects, the reporter is an electroactive reporter. In some aspects, the electroactive reporter comprises biotin and methylene blue. In some aspects, the reporter is an enzyme-nucleic acid. In some aspects, the enzyme-nucleic acid is an invertase enzyme. In some aspects, an enzyme of the enzyme-nucleic acid is a sterically hindered enzyme.

In some aspects, upon cleavage of a nucleic acid of the enzyme-nucleic acid, the enzyme is functional. In some aspects, the detectable signal is a colorimetric signal, a fluorescence signal, an amperometric signal, or a potentiometric signal.

In various aspects, the present disclosure provides a method of detecting a target nucleic acid in a sample comprising: contacting the sample with a guide nucleic acid targeting a target sequence; a programmable nuclease capable of being activated when complexed with the guide nucleic acid and the target sequence; and a reporter, wherein the reporter is capable of being cleaved by the activated nuclease, thereby generating a detectable signal.

In some aspects, the target nucleic acid is from an exogenous pathogen. In some aspects, the exogenous pathogen comprises a virus. In some aspects, the virus comprises an influenza virus. In some aspects, the influenza virus comprises an influenza A virus, influenza B virus, or a combination thereof. In some aspects, the virus comprises a respiratory virus. In some aspects, the respiratory virus is an upper respiratory virus.

In some aspects, the detectable signal indicates presence of the virus in the sample. In some aspects, the method further comprises diagnosing a subject from which the sample was taken with the virus. In some aspects, the subject is a human. In some aspects, the sample is a buccal swab, a nasal swab, or urine. In some aspects, the reporter comprises a single stranded reporter comprising a detection moiety. In some aspects, the guide nucleic acid targets a plurality of target sequences.

In some aspects, the system comprises a plurality of guide sequences tiled against the virus. In some aspects, the plurality of target sequences comprises sequences from influenza A virus, influenza B virus, and a third pathogen. In some aspects, the single stranded reporter comprises the detection moiety at the 5′ end. In some aspects, the single stranded reporter comprises a biotin-dT/FAM moiety or a biotin-dT/ROX moiety. In some aspects, the single stranded reporter comprises a chemical functional handle at the 3′end capable of being conjugated to a substrate. In some aspects, the substrate is a magnetic bead.

In some aspects, the substrate is a surface of a reaction chamber. In some aspects, downstream of the reaction chamber is a test line. In some aspects, the test line comprises a streptavidin. In some aspects, downstream of the test line is a flow control line. In some aspects, the flow control line comprises an anti-IgG antibody. In some aspects, the anti-IgG antibody comprises an anti-rabbit IgG antibody.

In some aspects, the activated nuclease is capable of cleaving the single stranded reporter and releases the biotin-dT/FAM moiety or the biotin-dT/ROX moiety. In some aspects, the biotin-dT/FAM moiety is capable of binding the streptavidin at the test line. In some aspects, the reporter is an electroactive reporter. In some aspects, the electroactive reporter comprises biotin and methylene blue. In some aspects, the reporter is an enzyme-nucleic acid. In some aspects, the enzyme-nucleic acid is an invertase enzyme. In some aspects, an enzyme of the enzyme-nucleic acid is a sterically hindered enzyme. In some aspects, upon cleavage of a nucleic acid of the enzyme-nucleic acid, the enzyme is functional. In some aspects, the detectable signal is a colorimetric signal, a fluorescence signal, an amperometric signal, or a potentiometric signal. In some aspects, in any of the above systems, the respiratory virus is a lower respiratory virus. In some aspects, in any of the above methods, the respiratory virus is a lower respiratory virus.

In some aspects, a composition comprises a DNA-activated programmable RNA nuclease; and a guide nucleic acid comprising a segment that is reverse complementary to a segment of a target deoxyribonucleic acid, wherein the DNA-activated programmable RNA nuclease binds to the guide nucleic acid to form a complex. In some aspects, the composition further comprises an RNA reporter. In some aspects, the composition further comprises the target deoxyribonucleic acid from a virus. In some aspects, the target deoxyribonucleic acid is an amplicon of a nucleic acid. In some aspects, wherein the nucleic acid is a deoxyribonucleic acid or a ribonucleic acid. In some aspects, the DNA-activated programmable RNA nuclease is a Type VI CRISPR/Cas enzyme. In some aspects, the DNA-activated programmable RNA nuclease is a Cas13. In some aspects, the DNA-activated programmable RNA nuclease is a Cas13a. In some aspects, the Cas13a is Lbu-Cas13a or Lwa-Cas13a. In some aspects, the composition has a pH from pH 6.8 to pH 8.2. In some aspects, the target deoxyribonucleic acid lacks a guanine at the 3′ end. In some aspects, the target deoxyribonucleic acid is a single-stranded deoxyribonucleic acid. In some aspects, the composition further comprises a support medium. In some aspects, the composition further comprises a lateral flow assay device. In some aspects, the composition further comprises a device configured for fluorescence detection. In some aspects, the composition further comprises a second guide nucleic acid and a DNA-activated programmable DNA nuclease, wherein the second guide nucleic acid comprises a segment that is reverse complementary to a segment of a second target deoxyribonucleic acid comprising a guide nucleic acid. In some aspects, the composition further comprises a DNA reporter. In some aspects, the DNA-activated programmable DNA nuclease is a Type V CRISPR/Cas enzyme. In some aspects, the DNA-activated programmable DNA nuclease is a Cas12. In some aspects, the Cas12 is a Cas12a, Cas12b, Cas12c, Cas12d, or Cas12e. In some aspects, the DNA-activated programmable DNA nuclease is a Cas14. In some aspects, the Cas14 is a Cas 14a, Cas14b, Cas14c, Cas 14d, Cas 14e, Cas 14f, Cas 14g, or Cas 14h.

In some aspects, a method of assaying for a target deoxyribonucleic acid from a virus in a sample comprises contacting the sample to a complex comprising a guide nucleic acid and a DNA-activated programmable RNA nuclease, wherein the guide nucleic acid comprises a segment that is reverse complementary to a segment of the target deoxyribonucleic acid, and

assaying for a signal produced by cleavage of at least some RNA reporters of a plurality of RNA reporters. In some aspects, a method of assaying for a target ribonucleic acid from a virus in a sample comprises: amplifying a nucleic acid in a sample to produce a target deoxyribonucleic acid; contacting the target deoxyribonucleic acid to a complex comprising a guide nucleic acid and a DNA-activated programmable RNA nuclease, wherein the guide nucleic acid comprises a segment that is reverse complementary to a segment of the target deoxyribonucleic acid, and assaying for a signal produced by cleavage of at least some RNA reporters of a plurality of RNA reporters. In some aspects, the DNA-activated programmable RNA nuclease is a Type VI CRISPR nuclease. In some aspects, the DNA-activated programmable RNA nuclease is a Cas13. In some aspects, the Cas13 is a Cas13a. In some aspects, the Cas13a is Lbu-Cas13a or Lwa-Cas13a. In some aspects, cleavage of the at least some RNA reporters of the plurality of reporters occurs from pH 6.8 to pH 8.2. In some aspects, the target deoxyribonucleic acid lacks a guanine at the 3′ end. In some aspects, the target deoxyribonucleic acid is a single-stranded deoxyribonucleic acid. In some aspects, the target deoxyribonucleic acid is an amplicon of a ribonucleic acid. In some aspects, the target deoxyribonucleic acid or the ribonucleic acid is from an organism. In some aspects, the organism is a virus, bacteria, plant, or animal. In some aspects, the target deoxyribonucleic acid is produced by a nucleic acid amplification method. In some aspects, the nucleic acid amplification method is isothermal amplification. In some aspects, the nucleic acid amplification method is thermal amplification. In some aspects, the nucleic acid amplification method is recombinase polymerase amplification (RPA), transcription mediated amplification (TMA), strand displacement amplification (SDA), helicase dependent amplification (HDA), loop mediated amplification (LAMP), rolling circle amplification (RCA), single primer isothermal amplification (SPIA), ligase chain reaction (LCR), simple method amplifying RNA targets (SMART), or improved multiple displacement amplification (IMDA), or nucleic acid sequence-based amplification (NASBA). In some aspects, the signal is fluorescence, luminescence, colorimetric, electrochemical, enzymatic, calorimetric, optical, amperometric, or potentiometric. In some aspects, the method further comprises contacting the sample to a second guide nucleic acid and a DNA-activated programmable DNA nuclease, wherein the second guide nucleic acid comprises a segment that is reverse complementary to a segment of a second target deoxyribonucleic acid comprising a guide nucleic acid. In some aspects, the method further comprises assaying for a signal produced by cleavage of at least some DNA reporters of a plurality of DNA reporters. In some aspects, the DNA-activated programmable DNA nuclease is a Type V CRISPR nuclease. In some aspects, the DNA-activated programmable DNA nuclease is a Cas12. In some aspects, the Cas12 is a Cas12a, Cas12b, Cas12c, Cas12d, or Cas12e. In some aspects, the DNA-activated programmable DNA nuclease is a Cas14. In some aspects, the Cas14 is a Cas14a, Cas14b, Cas14c, Cas14d, Cas14e, Cas14f, Cas14g, or Cas14h. In some aspects, the guide nucleic acid comprises a crRNA. In some aspects, the guide nucleic acid comprises a crRNA and a tracrRNA. In some aspects, the signal is present prior to cleavage of the at least some RNA reporters. In some aspects, the signal is absent prior to cleavage of the at least some RNA reporters. In some aspects, the sample comprises blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. In some aspects, the method is carried out on a support medium. In some aspects, the method is carried out on a lateral flow assay device. In some aspects, the method is carried out on a device configured for fluorescence detection.

In various aspects, the present disclosure provides a method of designing a plurality of primers for amplification of a target nucleic acid, the method comprising: providing a target nucleic acid, herein a guide nucleic acid hybridizes to the target nucleic acid and wherein at least 60% of a sequence of the target nucleic acid is between an F1c region and a B1 region or between an F1 and a B1c region; and designing the plurality of primers comprising: i) a forward inner primer comprising a sequence of the F1c region 5′ of a sequence of an F2 region; ii) a backward inner primer comprising a sequence of the B1c region 5′ of a sequence of a B2 region; iii) a forward outer primer comprising a sequence of an F3 region; and iv) a backward outer primer comprising a sequence of a B3 region.

In various aspects, the present disclosure provides a method of detecting a target nucleic acid in a sample, the method comprising: contacting the sample to: a plurality of primers comprising: i) a forward inner primer comprising a sequence corresponding to an F1c region 5′ of a sequence corresponding to an F2 region; ii) a backward inner primer comprising a sequence corresponding to a B1c region 5′ of a sequence corresponding to a B2 region; iii) a forward outer primer comprising a sequence corresponding to an F3 region; and iv) a backward outer primer comprising a sequence corresponding to a B3 region; a guide nucleic acid, wherein the guide nucleic acid hybridizes to the target nucleic acid and wherein at least 60% of a sequence of the target nucleic acid is between the F1c region and a B1 region or between an F1 region and the B1c region; a reporter; and a programmable nuclease that cleaves the reporter when complexed with the guide nucleic acid; and

measuring a detectable signal produced by cleavage of the reporter, wherein the measuring provides for detection of the target nucleic acid in the sample.

In some aspects, the sequence between the F1c region and the B1 region or the sequence between the B1c region and the F1 region is at least 50% reverse complementary to the guide nucleic acid sequence. In some aspects, the guide nucleic acid sequence is reverse complementary to no more than 50% of the forward inner primer, the backward inner primer, or a combination thereof. In some aspects, the guide nucleic acid does not hybridize to the forward inner primer and the backward inner primer.

In some aspects, a protospacer adjacent motif (PAM) or a protospacer flanking site (PFS) is 3′ of the target nucleic acid. In some aspects, a protospacer adjacent motif (PAM) or a protospacer flanking site (PFS) is 3′ of the B1 region and 5′ of the F1c region or the protospacer adjacent motif (PAM) or a protospacer flanking site (PFS) is 3′ of the Fl region and 5′ of the B1c region. In some aspects, the 3′ end of the target nucleic acid is 5′ of the 5′ end of the F3c region or the 3′ end of the target nucleic acid is 5′ of the 5′ end of the B3c region. In some aspects, the 3′ end of the target nucleic acid is 5′ of the 5′ end of the F2c region or 3′ end of the target nucleic acid is 5′ of the 5′ end of the B2c region. In some aspects, the target nucleic acid is between the F1c region and the B1 region and the 3′ end of the target nucleic acid is 5′ of the 3′ end of the F2c region, or wherein the target nucleic acid is between the B1c region and the F1 region and the 3′ end of the target nucleic acid is 5′ of the 3′ end of the B2c region.

In some aspects, the guide nucleic acid has a sequence reverse complementary to no more than 50% of the forward inner primer, the backward inner primer, the forward outer primer, the backward outer primer, or any combination thereof. In some aspects, the guide nucleic acid sequence does not hybridize to the forward inner primer, the backward inner primer, the forward outer primer, the backward outer primer, or any combination thereof.

In some aspects, the guide nucleic acid sequence has a sequence reverse complementary to no more than 50% of a sequence of an F3c region, an F2c region, the F1c region, the B1c region, an B2c region, an B3c region, or any combination thereof. In some aspects, the guide nucleic acid sequence does not hybridize to a sequence of an F3c region, an F2c region, the F1c region, the B1c region, an B2c region, an B3c region, or any combination thereof.

In various aspects, the present disclosure provides a method of designing a plurality of primer for amplification of a target nucleic acid, the method comprising: providing the target nucleic acid comprising a sequence between a B2 region and a B1 region or between an F2 region and an F1 region that hybridizes to a guide nucleic acid; and designing the plurality of primers comprising: i) a forward inner primer comprising a sequence of the F1c region 5′ of a sequence of an F2 region; ii) a backward inner primer comprising a sequence of the B1c region 5′ of a sequence of a B2 region; iii) a forward outer primer comprising a sequence of an F3 region; and iv) a backward outer primer comprising a sequence of a B3 region.

In various aspects, the present disclosure provides a method of designing a plurality of primer for amplification of a target nucleic acid, the method comprising: providing the target nucleic acid comprising a sequence between a F1c region and an F2c region or between a B1c region and a B2c region that hybridizes to a guide nucleic acid; and designing the plurality of primers comprising: i) a forward inner primer comprising a sequence of the F1c region 5′ of a sequence of an F2 region; ii) a backward inner primer comprising a sequence of the B1c region 5′ of a sequence of a B2 region; iii) a forward outer primer comprising a sequence of an F3 region; and iv) a backward outer primer comprising a sequence of a B3 region.

In various aspects, the present disclosure provides a method of detecting a target nucleic acid in a sample, the method comprising: contacting the sample to: a plurality of primers comprising: i) a forward inner primer comprising a sequence corresponding to an F1c region 5′ of a sequence corresponding to an F2 region; ii) a backward inner primer comprising a sequence corresponding to a B1c region 5′ of a sequence corresponding to a B2 region; iii) a forward outer primer comprising a sequence corresponding to an F3 region; and iv) a backward outer primer comprising a sequence corresponding to a B3 region; a guide nucleic acid, wherein the target nucleic acid comprises a sequence between a B2 region and a B1 region or between the F2 region and an F1 region that hybridizes to the guide nucleic acid; a reporter; and a programmable nuclease that cleaves the reporter when complexed with the guide nucleic acid; and measuring a detectable signal produced by cleavage of the reporter, wherein the measuring provides for detection of the target nucleic acid in the sample.

In various aspects, the present disclosure provides a method of detecting a target nucleic acid in a sample, the method comprising: contacting the sample to: a plurality of primers comprising: i) a forward inner primer comprising a sequence corresponding to an F1c region 5′ of a sequence corresponding to an F2 region; ii) a backward inner primer comprising a sequence corresponding to a B1c region 5′ of a sequence corresponding to a B2 region; iii) a forward outer primer comprising a sequence corresponding to an F3 region; and iv) a backward outer primer comprising a sequence corresponding to a B3 region; a guide nucleic acid, wherein the target nucleic acid comprises a sequence between the F1c region and an F2c region or between the B1c region and a B2c region that hybridizes to the guide nucleic acid; a reporter; and a programmable nuclease that cleaves the reporter when complexed with the guide nucleic acid; and measuring a detectable signal produced by cleavage of the reporter, wherein the measuring provides for detection of the target nucleic acid in the sample.

In some aspects, a protospacer adjacent motif (PAM) or a protospacer flanking site (PFS) is 3′ of the B2 region and 5′ of the B1 region or the protospacer adjacent motif (PAM) or a protospacer flanking site (PFS) is 3′ of the F2 region and 5′ of the F1 region. In some aspects, a protospacer adjacent motif (PAM) or a protospacer flanking site (PFS) is 3′ of the B1c region and 5′ of the B2c region or the protospacer adjacent motif (PAM) or a protospacer flanking site (PFS) is 3′ of the F1c region and 5′ of the F2c region.

In some aspects, a protospacer adjacent motif (PAM) or a protospacer flanking site (PFS) is 3′ of the target nucleic acid. In some aspects, the PAM and the PFS are 5′ of the 5′ end of the F1c region, 5′ of the 5′ end of the B1c region, 3′ of the 3′ end of the F3 region, 3′ of the 3′ end of the B3 region, 3′ of the 3′ end of the F2 region, 3′ of the 3′ end of the B2 region, or any combination thereof

In some aspects, the PAM and the PFS do not overlap the F2 region, the B3 region, the F1c region, the F2 region, the B1c region, the B2 region, or any combination thereof. In some aspects, the PAM and the PFS do not hybridize to the forward inner primer, the backward inner primer, the forward outer primer, the backward outer primer, or any combination thereof.

In some aspects, the plurality of primers further comprises a loop forward primer. In some aspects, the plurality of primers further comprises a loop backward primer. In some aspects, the loop forward primer is between an F1c region and an F2c region. In some aspects, the loop backward primer is between a B1c region and a B2c region.

In some aspects, the target nucleic acid comprises a single nucleotide polymorphism (SNP). In some aspects, the single nucleotide polymorphism (SNP) comprises a HERC2 SNP. In some aspects, the single nucleotide polymorphism (SNP) is associated with an increased risk or decreased risk of cancer. In some aspects, the target nucleic acid comprises a single nucleotide polymorphism (SNP), and wherein the detectable signal is higher in the presence of a guide nucleic acid that is 100% complementary to the target nucleic acid comprising the single nucleotide polymorphism (SNP) than in the presence of a guide nucleic acid that is less than 100% complementary to the target nucleic acid comprising the single nucleotide polymorphism (SNP).

In some aspects, the plurality of primers and the guide nucleic acid are present together in a sample comprising the target nucleic acid. In some aspects, the contacting the sample to the plurality of primers results in amplifying the target nucleic acid. In some aspects, the amplifying and the contacting the sample to the guide nucleic acid occurs at the same time. In other aspects, the amplifying and the contacting the sample to the guide nucleic acid occur at different times. In some aspects, the method further comprises providing a polymerase, a dATP, a dTTP, a dGTP, a dCTP, or any combination thereof.

In some aspects, the target nucleic acid is from a virus. In some aspects, the virus comprises an influenza virus, respiratory syncytial virus, or a combination thereof. In further aspects, the influenza virus comprises an influenza A virus, influenza B virus, or a combination thereof. In some aspects, the virus comprises a respiratory virus. In further aspects, the respiratory virus is an upper respiratory virus.

In some aspects, the system further comprises a forward inner primer, a backward inner primer, a forward outer primer, a backward outer primer, a loop forward primer, a loop backward primer, or any combination thereof. In some aspects, method further comprising contacting the sample with a forward inner primer, a backward inner primer, a forward outer primer, a backward outer primer, a loop forward primer, a loop backward primer, or any combination thereof. In some aspects, method further comprising amplifying the target deoxyribonucleic acid with a forward inner primer, a backward inner primer, a forward outer primer, a backward outer primer, a loop forward primer, a loop backward primer, or any combination thereof. In some aspects, the amplifying comprises contacting the sample to a forward inner primer, a backward inner primer, a forward outer primer, a backward outer primer, a loop forward primer, a loop backward primer, or any combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a schematic illustrating a workflow of a CRISPR-Cas reaction. Step 1 shown in the workflow is sample preparation, Step 2 shown in the workflow is nucleic acid amplification. Step 3 shown in the workflow is Cas reaction incubation. Step 4 shown in the workflow is detection (readout). Non-essential steps are shown as oval circles. Steps 1 and 2 are not essential, and steps 3 and 4 can occur concurrently, if detection and readout are incorporated to the CRISPR reaction.

FIG. 2 shows an example fluidic device for sample preparation that may be used in Step 1 of the workflow schematic of FIG. 1. The sample preparation fluidic device shown in this figure can process different types of biological sample: finger-prick blood, urine or swabs with fecal, cheek or other collection.

FIG. 3 shows three example fluidic devices for a Cas reaction with a fluorescence or electrochemical readout that may be used in Step 2 to Step 4 of the workflow schematic of FIG. 1. This figure shows that the device performs three iterations of Steps 2 through 4 of the workflow schematic of FIG. 1.

FIG. 4 shows schematic diagrams of a readout process that may be used including (a) fluorescence readout and (b) electrochemical readout.

FIG. 5 shows an example fluidic device for coupled invertase/Cas reactions with colorimetric or electrochemical/glucometer readout. This diagram illustrates a fluidic device for miniaturizing a Cas reaction coupled with the enzyme invertase. Surface modification and readout processes are depicted in exploded view schemes at the bottom including (a) optical readout using DNS, or other compound and (b) electrochemical readout (electrochemical analyzer or glucometer).

FIG. 6A shows a panel of gRNAs for RSV evaluated for detection efficiency. Darker squares in the background subtracted row indicate greater efficiency of detecting RSV target nucleic acids.

FIG. 6B shows graphs of pools of gRNA versus background subtracted fluorescence.

FIG. 7 shows individual parts of sample preparation devices of the present disclosure.

FIG. 8 shows a sample work flow using a sample processing device.

FIG. 9 shows extraction buffers used to extract Influenza A RNA from remnant clinical samples.

FIG. 10 shows that low pH conditions allow for rapid extraction of Influenza A genomic RNA.

FIG. 11 shows the application of RT-RPA to the detection of Influenza A, Influenza B, and human Respiratory Syncytial Virus (RSV) viral RNA by Cas12a. The schematic at left shows the workflow including providing DNA/RNA, RPA/RT-RPA, and Cas12a detection. The graphs at right show the results of Cas12a detection as measured by fluorescence over time.

FIG. 12 shows the application of RT-RPA coupled with an IVT reaction enabling detection of viral RNA using Cas13a. The schematic at left shows the workflow including providing DNA/RNA, RPA/RT-RPA, in vitro transcription, and Cas13a detection. The graph at right shows the results of Cas13a detection as measured by fluorescence for each tested condition.

FIG. 13 shows the production of RNA, as detected by Cas13a, from an RNA virus using an RT-RPA-IVT “two-pot” reaction. The schematic at left shows the workflow including providing DNA/RNA, the “two-pot” reaction including RPA/RT-RPA and in vitro transcription in a first reaction, and Cas13a detection in a second reaction. The graph at right shows the results of Cas13a detection as measured by fluorescence for each tested condition.

FIG. 14 shows the effect of various buffers on the performance of a one-pot Cas13a assay. The schematic at left shows the workflow including providing DNA/RNA and RPA/RT-RPA, in vitro transcription, and Cas13a detection. The graph at right shows the results of Cas13a detection as measured by fluorescence for each tested condition.

FIG. 15 shows the specific detection of viral RNA from the Peste des petits ruminants (PPR) virus that infects goats using the one-pot Cas13a assay. The schematic at left shows the workflow including providing DNA/RNA and RPA/RT-RPA, in vitro transcription, and Cas13a detection. The graphs at right show the results of Cas13a detection as measured by fluorescence over time for the tested conditions.

FIG. 16 shows the specific detection of Influenza B using the one-pot Cas13a assay run at 40° C. 40 fM of viral RNA was added to the reaction. The schematic at left shows the workflow including providing DNA/RNA and RPA/RT-RPA, in vitro transcription, and Cas13a detection. The graphs at right show the results of Cas13a detection as measured by fluorescence for each tested condition.

FIG. 17 shows the tolerance of the one-pot Cas13a assay for the detection of RNA from the Influenza B virus in the presence and in the absence of a universal viral transport medium called universal transport media (UTM Copan) at 40° C. The schematic at left shows the workflow including providing DNA/RNA and RPA/RT-RPA, in vitro transcription, and Cas13a detection. The graphs at right show the results of Cas13a detection as measured by fluorescence over time for each tested condition.

FIGS. 18A-18D show the one-pot Cas13a detection assay at various temperatures.

FIG. 18A shows a schematic of the workflow including providing DNA/RNA and the one-pot reaction including RPA/RT-RPA, in vitro transcription, and Cas13a detection.

FIG. 18B shows a graph of Cas13a detection of Influenza A RNA at various temperatures.

FIG. 18C shows a graph of Cas13a detection of Influenza B RNA at various temperatures.

FIG. 18D shows a graph of Cas13a detection of human RSV RNA at various temperatures.

FIGS. 19A-19C show the optimization of a LAMP reaction for the detection of an internal amplification control using a DNA sequence derived from the Mammuthus primigenius (Wooly Mammoth) mitochondria.

FIG. 19A shows a schematic of the workflow including providing DNA/RNA, LAMP/RT-LAMP, and Cas12a detection.

FIG. 19B shows the time to result for LAMP reactions for an internal amplification control using a DNA sequence derived from the Mammuthus primigenius, as quantified by fluorescence.

FIG. 19C shows Cas12a specific detection at 37° C. of LAMP amplicon from the 68° C. temperature reaction.

FIGS. 20A-20C show the optimization of LAMP and Cas12 specific detection of the human POP7 gene that is a component of RNase P (SEQ ID NO: 379,

GGAGTATTGAATAGTTGGGAATTGGAACCCCTCCAGGGGGAACCAAACAT
TGTCGTTCAGAAGAAGACAAAGAGAGATTGAAATGAAGCTGTTGATTTCA
ACACACAAATTCTGGTGGTAGATGAAAGCAAAGCAAGTAAGTTTCTCCGA
ATCCCTAGTCAACTGGAGGTAGAGACGGACTGCGCAGGTTAACTACAGCT
CCCAGCATGCCTGAGGGGCGGGCTCAGCGGCTGCGCAGACTGGCGCGCGC
GGACGGTCATGGGACTTCAGCATGGCGGTGTTTGCAGATTTGGACCTGCG
AGCGGGTTCTGACCTGAAGGCTCTGCGCGGACTTGTGGAGACAGCCGCTC
ACCTTGGCTATTCAGTTGTTGCTATCAATCATATCGTTGACTTTAAGGAA
AAGAAACAGGAAATTGAAAAACCAGTAGCTGTTTCTGAACTCTTCACAAC
TTTGCCAATTGTACAGGGAAAATCAAGACCAATTAAAATTTTAACTAGAT
TAACAATTATTGTCTCGGATCCATCTCACTGCAATGTTTTGAGAGCAACT
TCTTCAAGGGCCCGGCTCTATGATGTTGTTGCAGTTTTTCCAAAGACAGA
AAAGCTTTTTCATATTGCTTGCACACATTTAGATGTGGATTTAGTCTGCA
TAACTGTAACAGAGAAACTACCATTTTACTTCAAAAGACCTCCTATTAAT
GTGGCGATTGACCGAGGCCTGGCTTTTGAACTTGTCTATAGCCCTGCTAT
CAAAGACTCCACAATGAGAAGGTATACAATTTCCAGTGCCCTCAATTTGA
TGCAAATCTGCAAAGGAAAGAATGTAATTATATCTAGTGCTGCAGAAAGG
CCTTTAGAAATAAGAGGGCCATATGACGTGGCAAATCTAGGCTTGCTGTT
TGGGCTCTCTGAAAGTGACGCCAAGGCTGCGGTGTCCACCAACTGCCGAG
CAGCGCTTCTCCATGGAGAAACTAGAAAAACTGCTTTTGGAATTATCTCT
ACAGTGAAGAAACCTCGGCCATCAGAAGGAGATGAAGATTGTCTTCCAGC
TTCCAAGAAAGCCAAGTGTGAGGGCTGAAAAGAATGCCCCAGTCTCTGTC
AGCACTCCCTTCTTCCCTTTTATAGTTCATCAGCCACAACAAAAATAAAA
CCTTTGTGTGATTTACTGTTTTCATTTGGAGCTAGAAATCAATAGTCTAT
AAAAACAGTTTTACTTGCAATCCATTAAAACAACAAACGAAACCTAGTGA
AGCATCTTTTTAAAAGGCTGCCAGCTTAATGAATTTAGATGTACTTTAAG
AGAGAAAGACTGGTTATTTCTCCTTTGTGTAAGTGATAAACAACAGCAAA
TATACTTGAATAAAATGTTTCAGGTATTTTTGTTTCATTTTGTTTTTGAG
ATAGGGTCTTTGTTGCTCAGGCTGGAGTACAGTGGCATAATCACAGCTCA
CTGCAACCTCAATCCTGGGCTCAAGTGATCCTCCCGCTTCAGCCTCTCAA
GCAGCGGGAACTACAGGTGTGCACTACCACACCTGGCTATTTTTTTTTTT
TTTTTTTTTTTCCCTTGTAGAGACATGGTCTCACTATGTTGCTGAGGCTG
GTCTCAAACTCCTAGGATCAAGCCATCCTCCCGCTTTGGCCTCCTAAAGT
GCTGGGATTACATGAGCCACCACATGCAGCCAGATGTTTGAATATTTTAA
GAGCTTCTTTCGAAAGTTTCTTGTTCATACTCAAATAGTAGTTATTTTGA
AGATATTCAAACTTATATTGAAGAAGTGACTTTAGTTCCTCTTGTTTTAA
GCTTCTTTCATGTATTCAAATCAGCATTTTTTTCTAAGAAATTGCTATAG
AATTTGTGGAAGGAGAGAGGATACACATGTAAAATTACATCTGGTCTCTT
CCTTCACTGCTTCATGCCTACGTAAGGTCTTTGAAATAGGATTCCTTACT
TTTAGTTAGAAACCCCTAAAACGCTAATATTGATTTTCCTGATAGCTGTA
TTAAAAATAGCAAAGCATCGGACTGA).

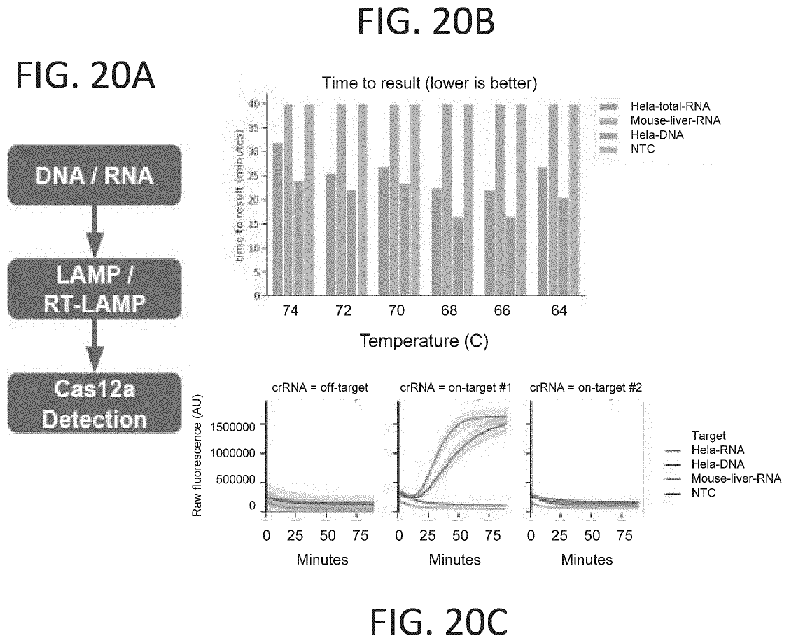

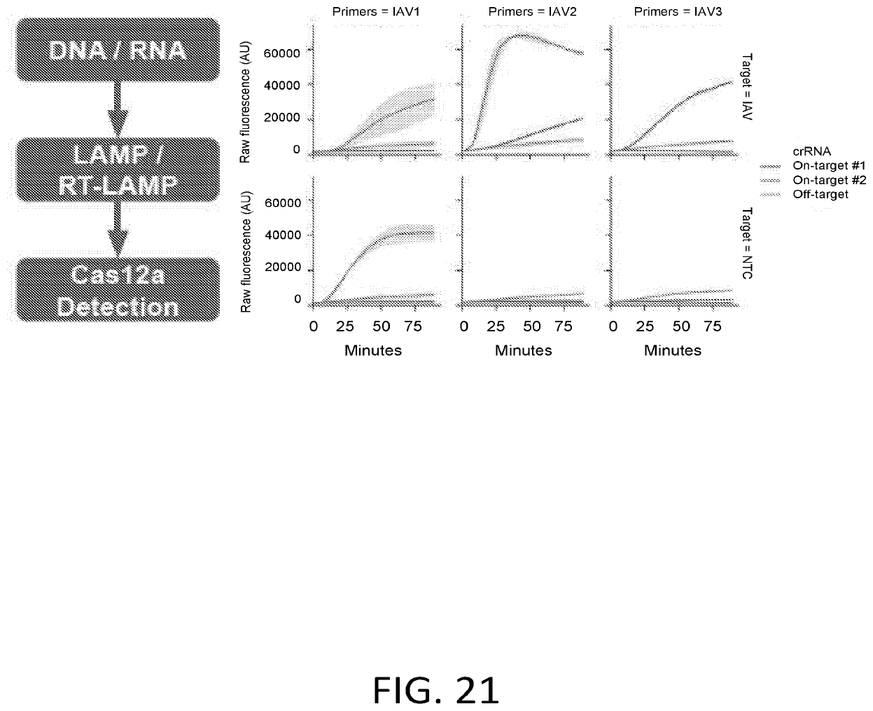

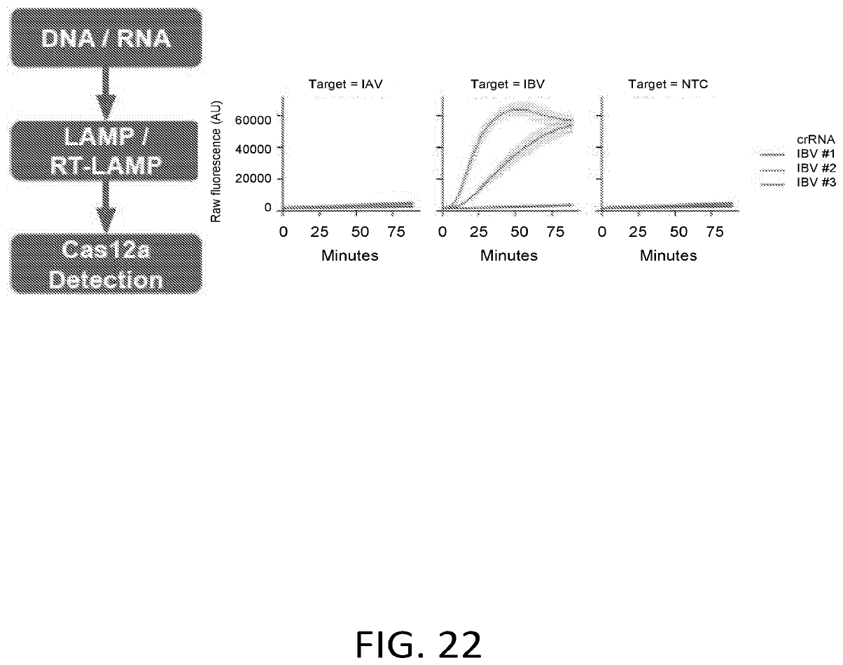

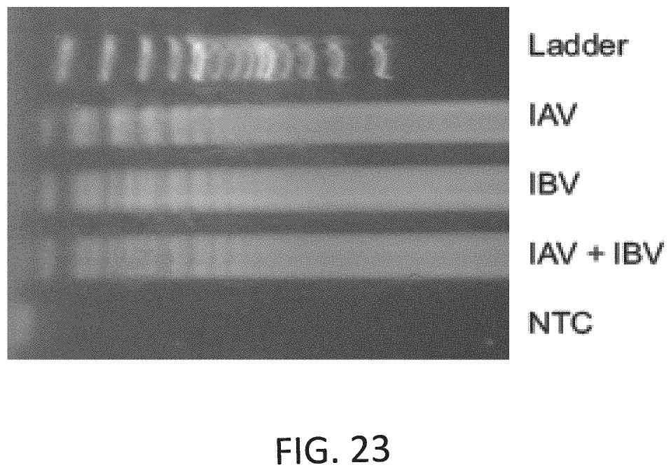

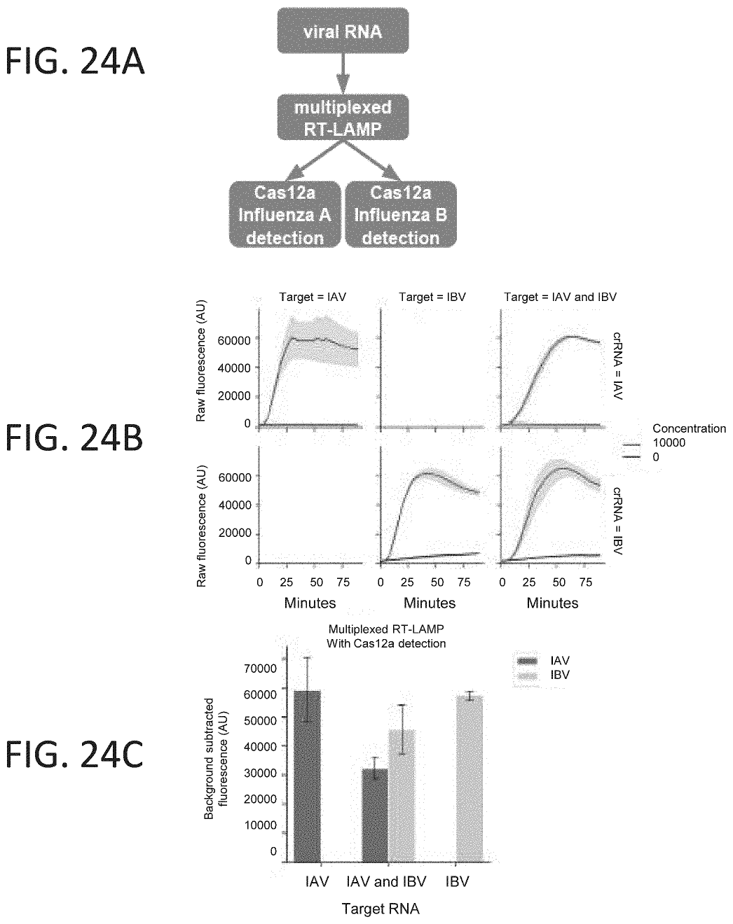

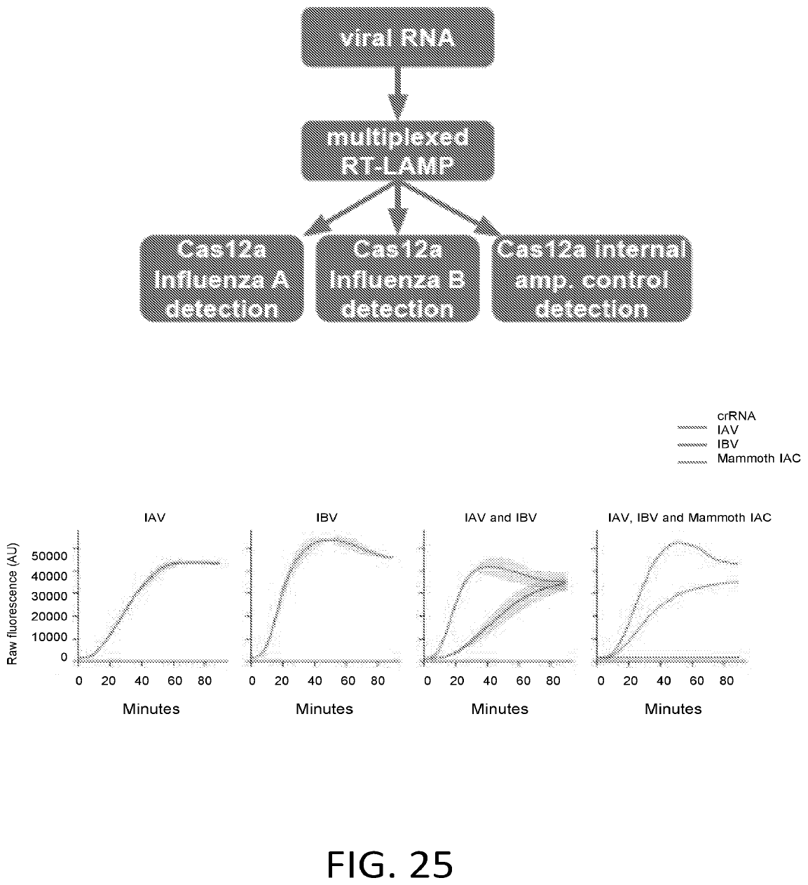

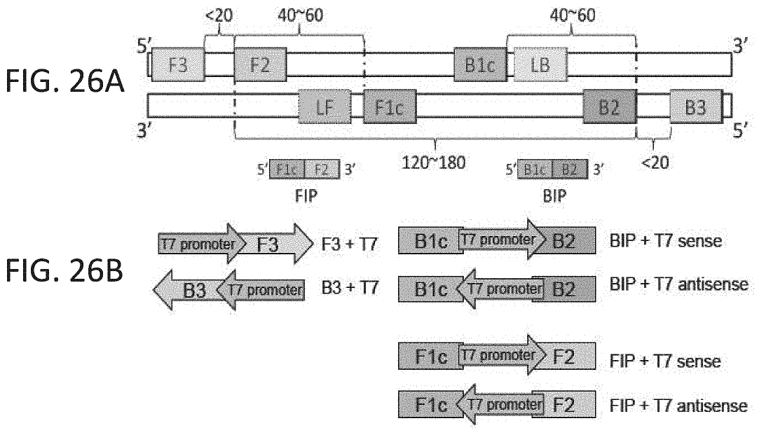

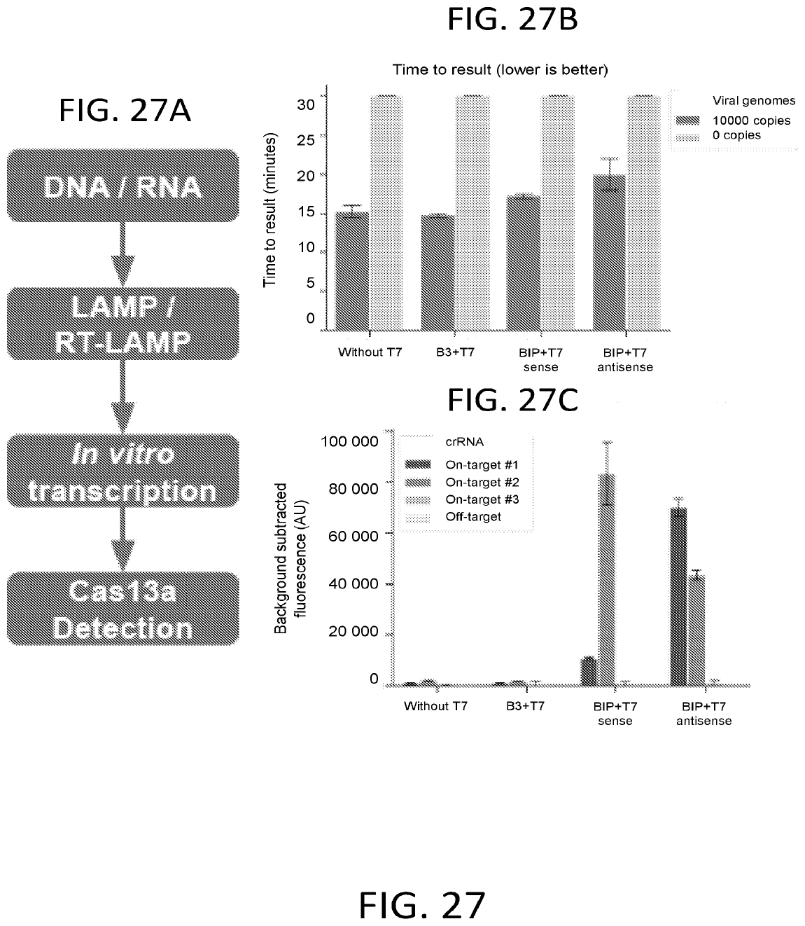

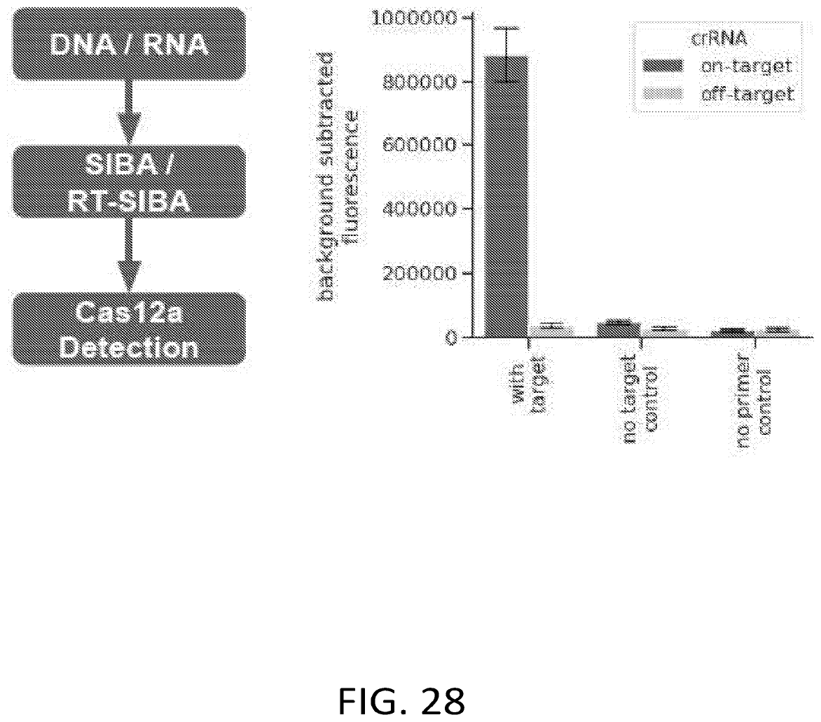

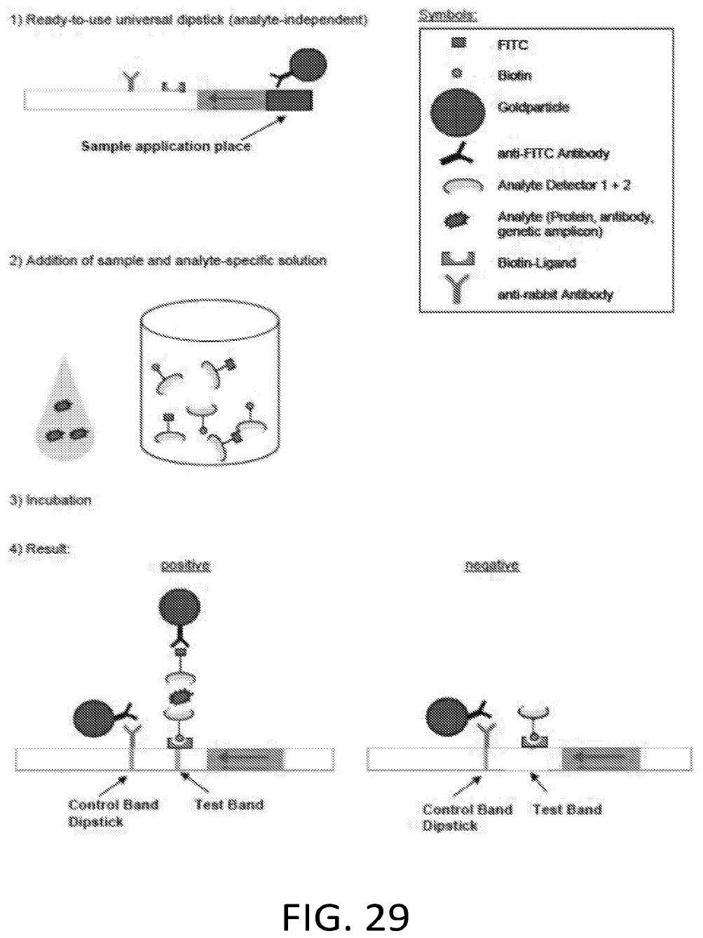

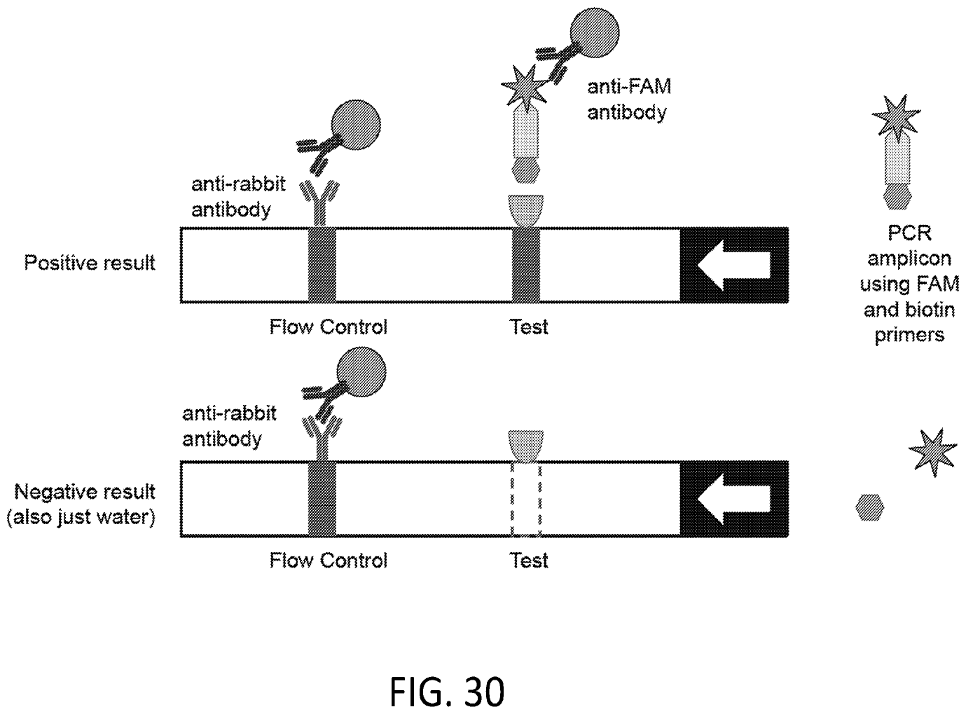

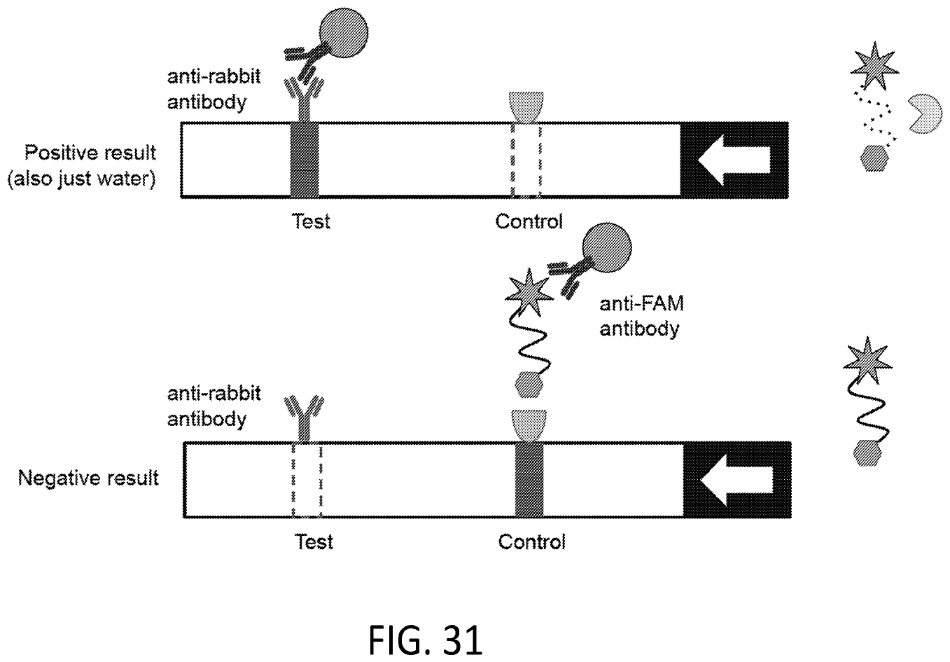

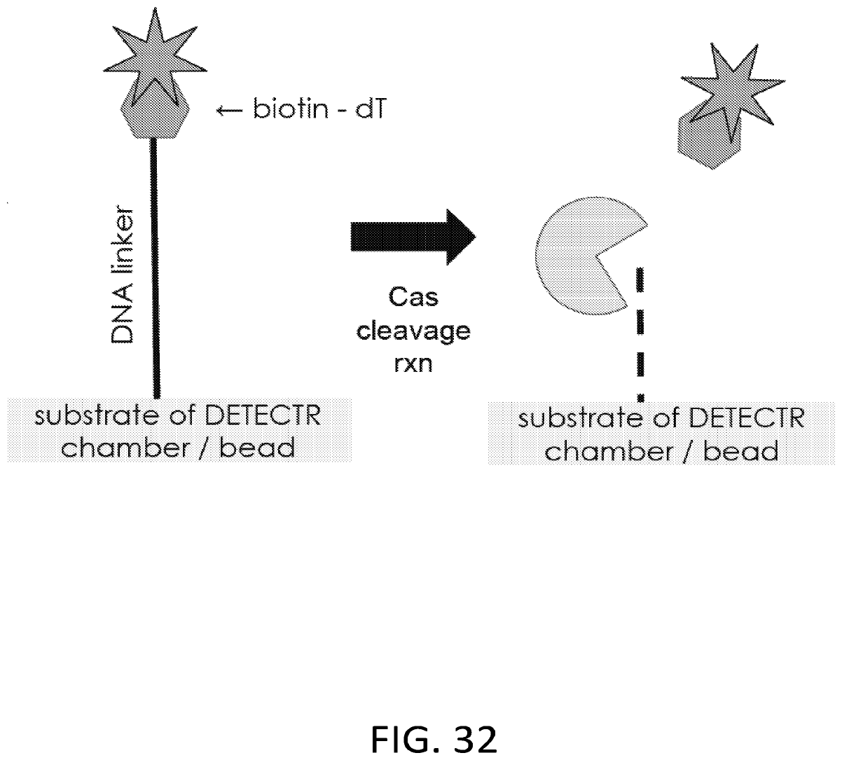

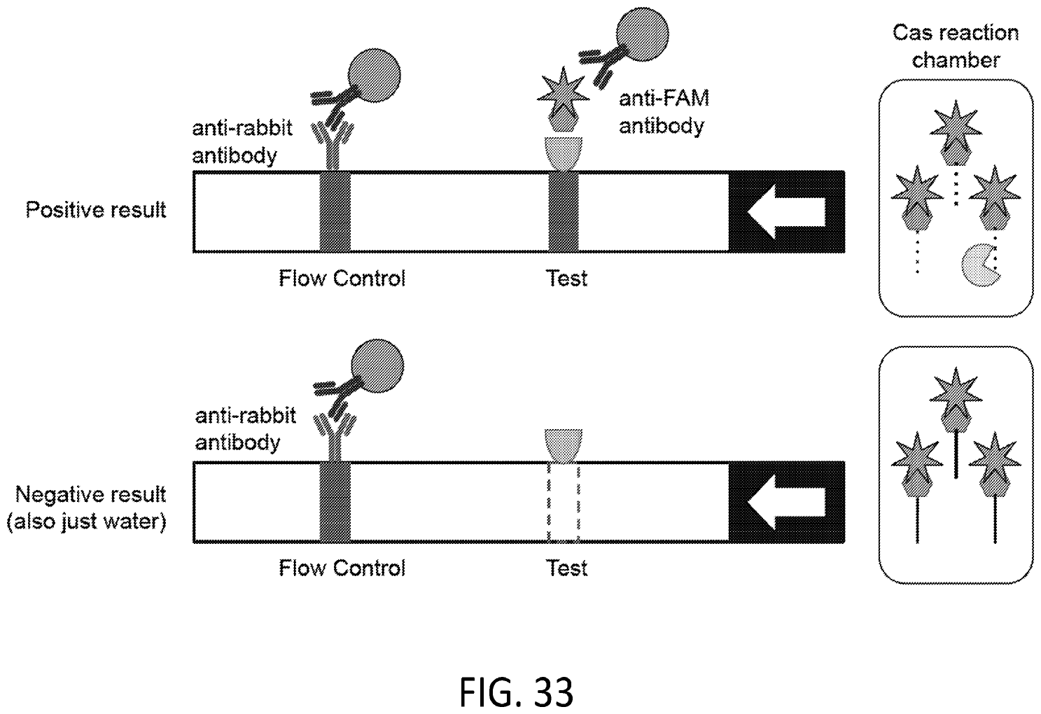

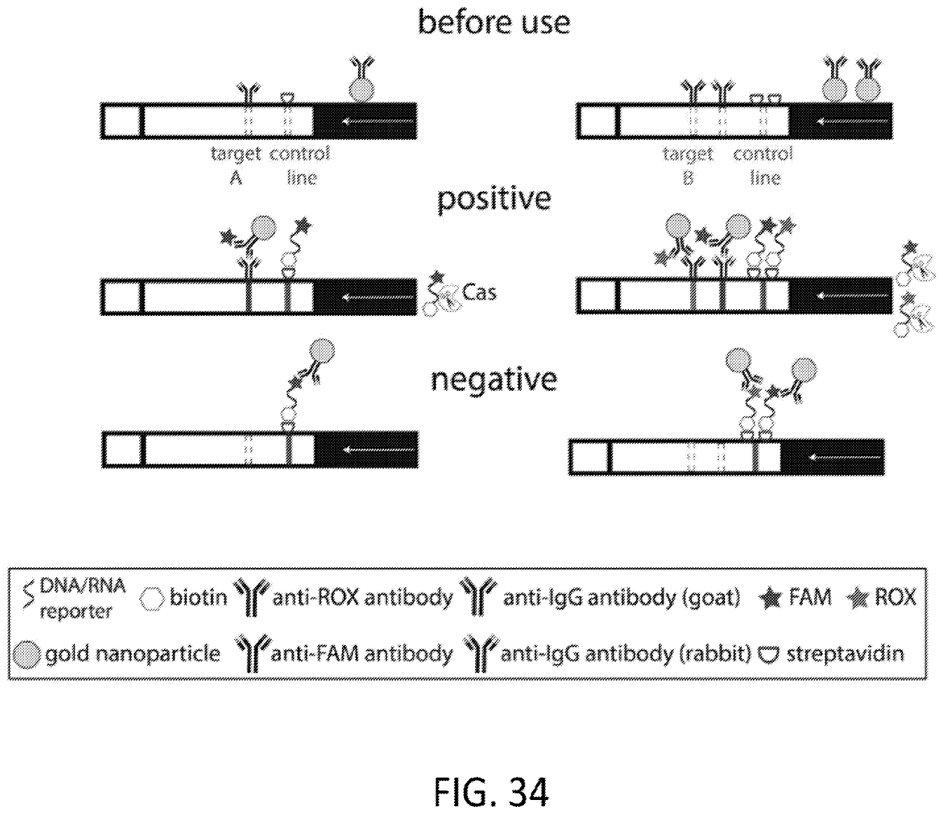

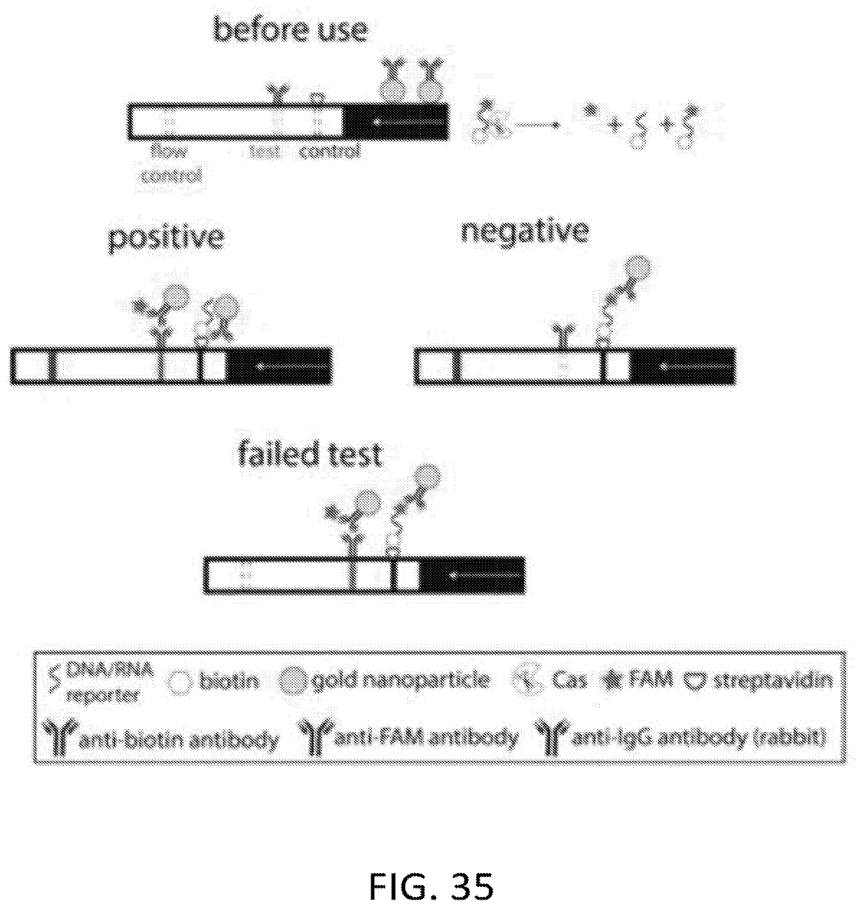

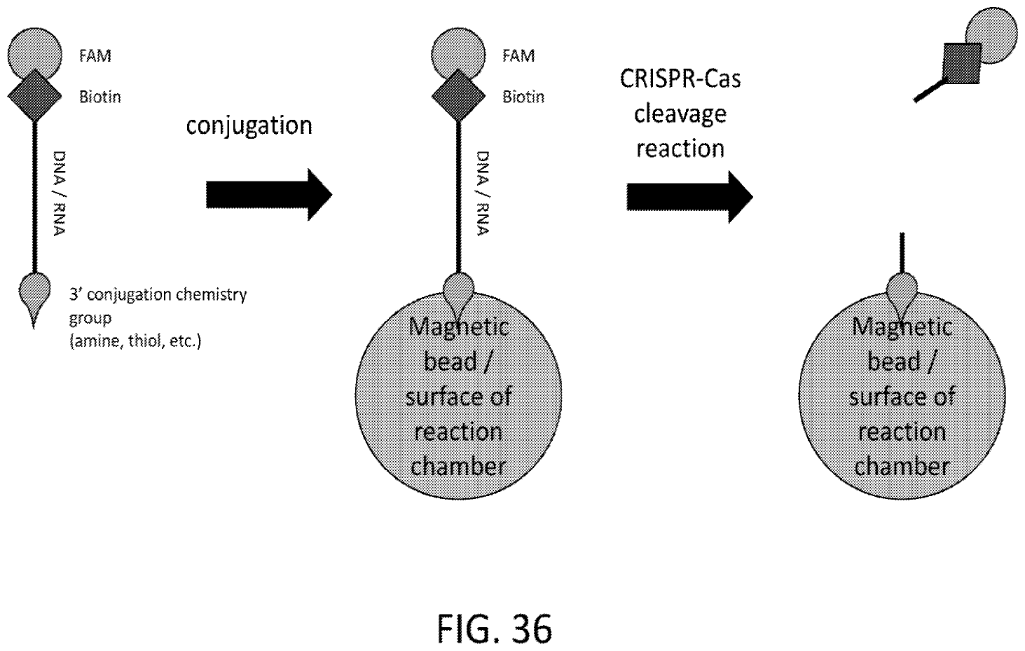

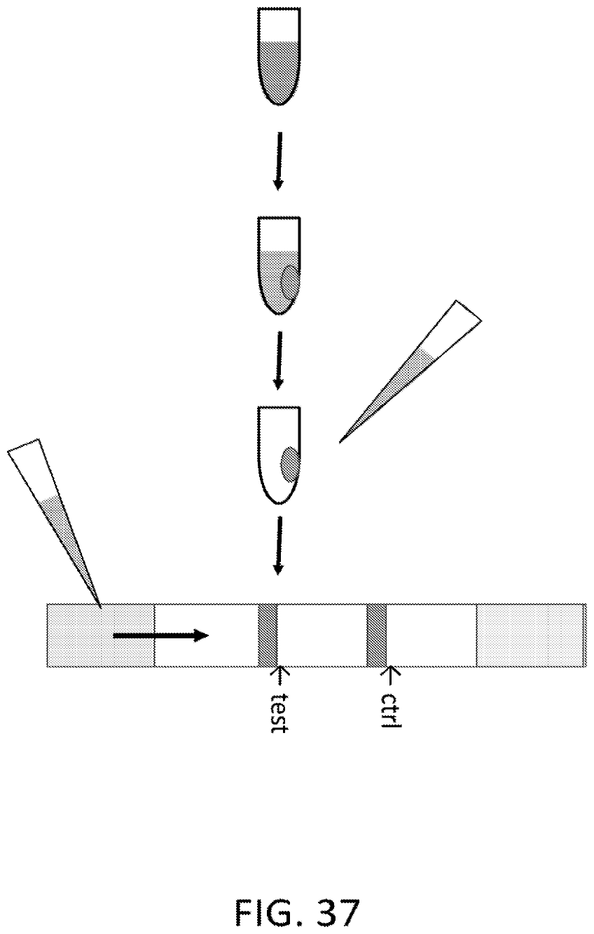

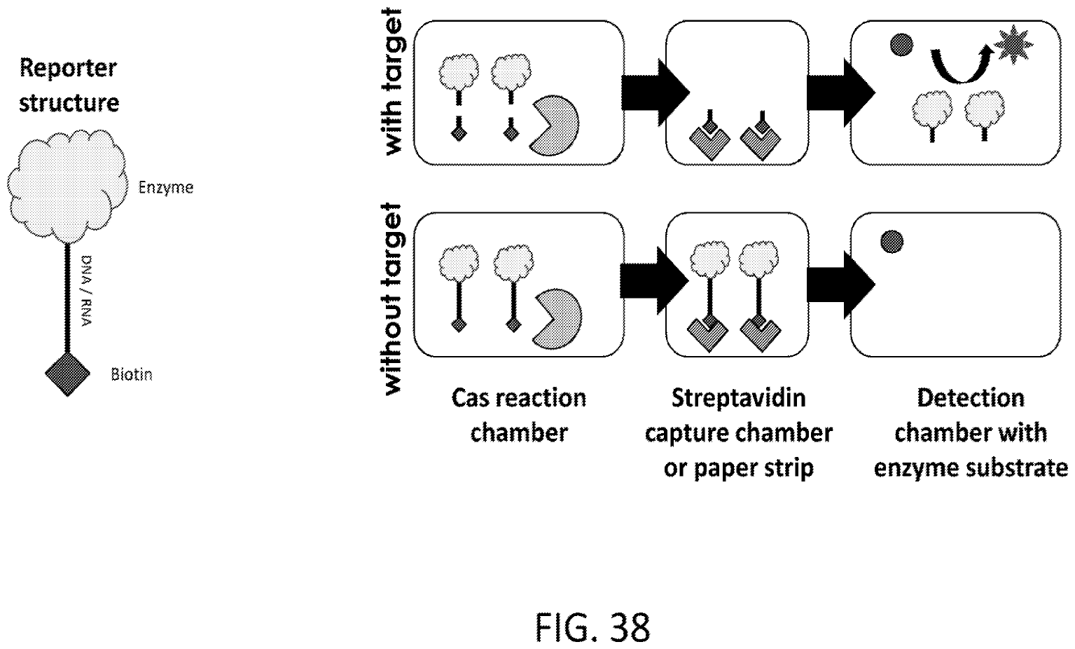

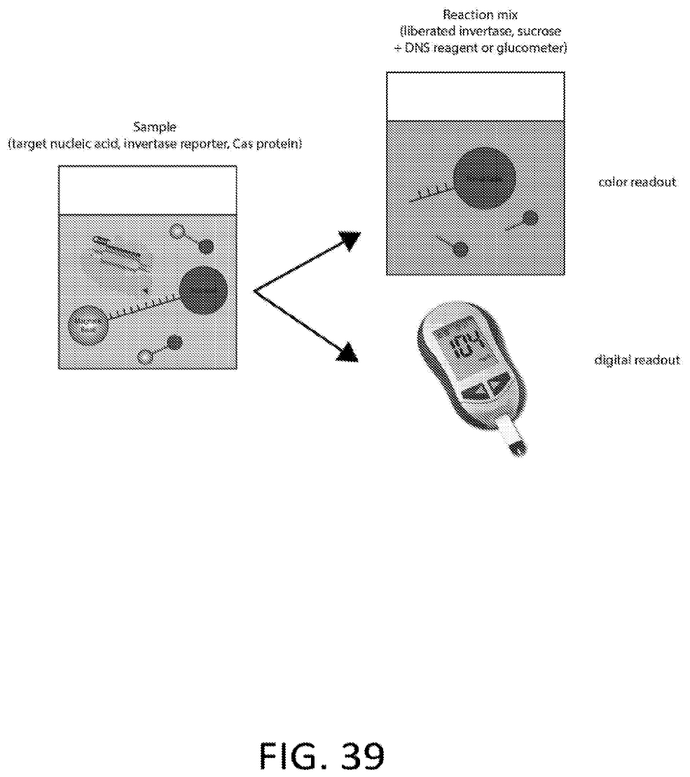

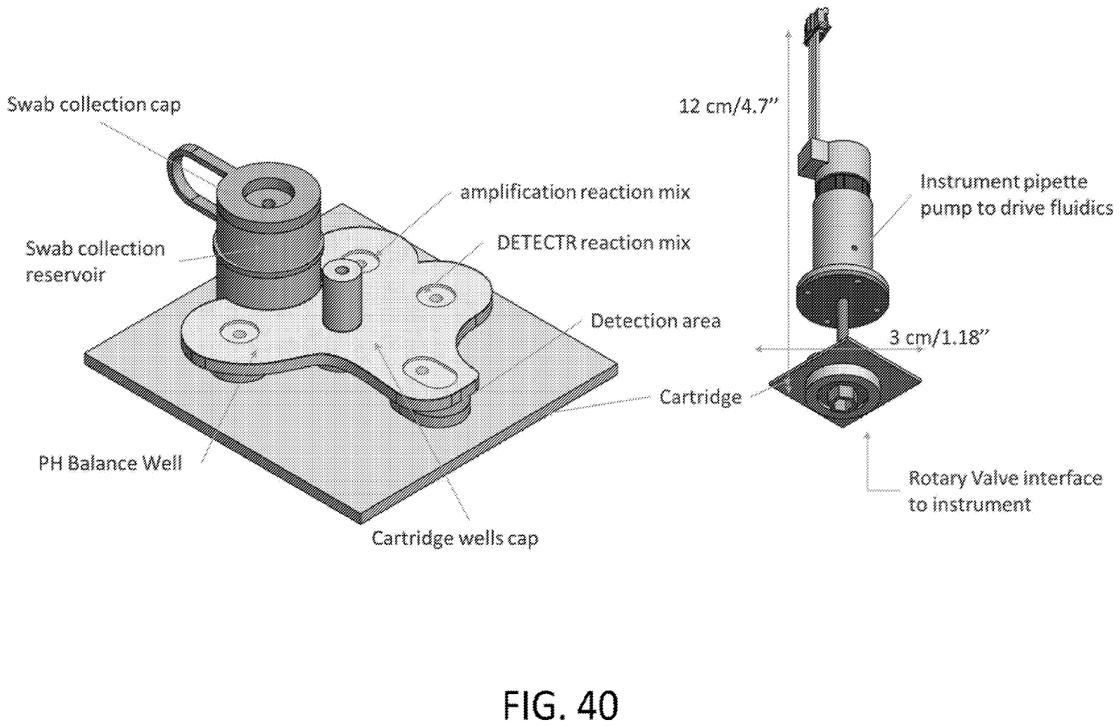

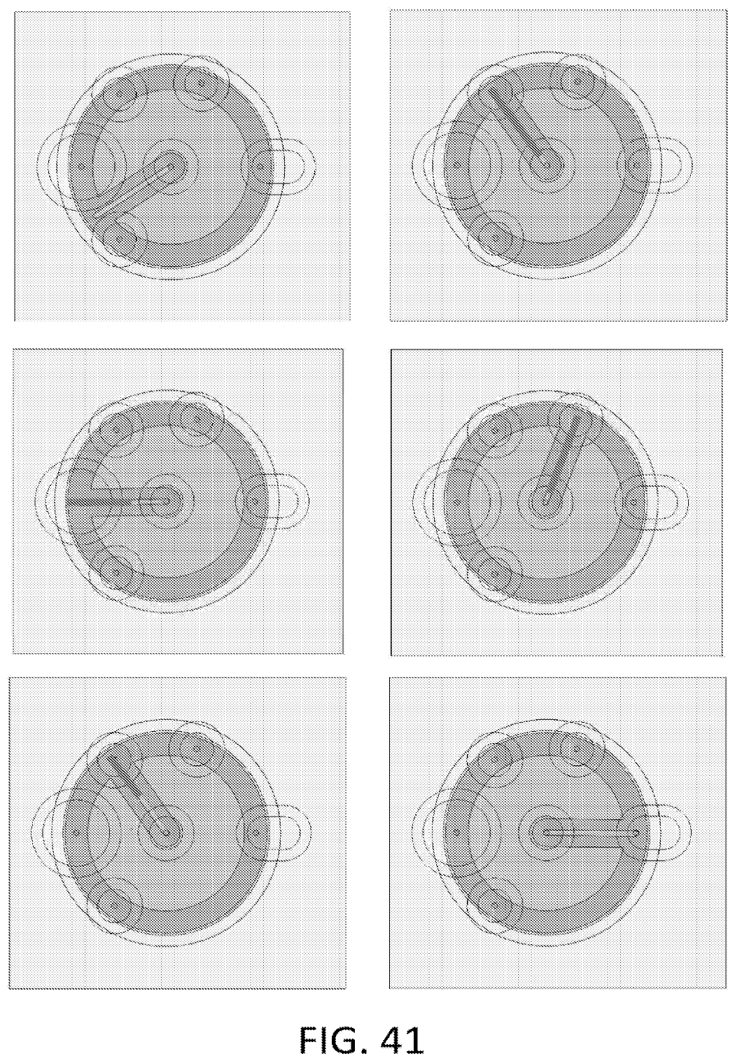

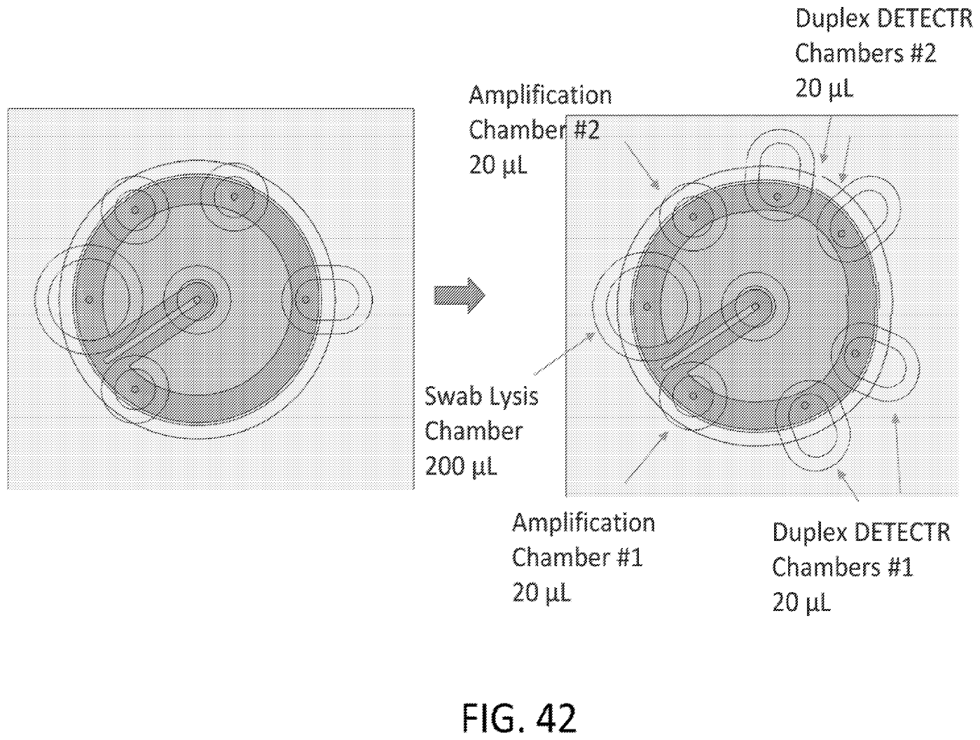

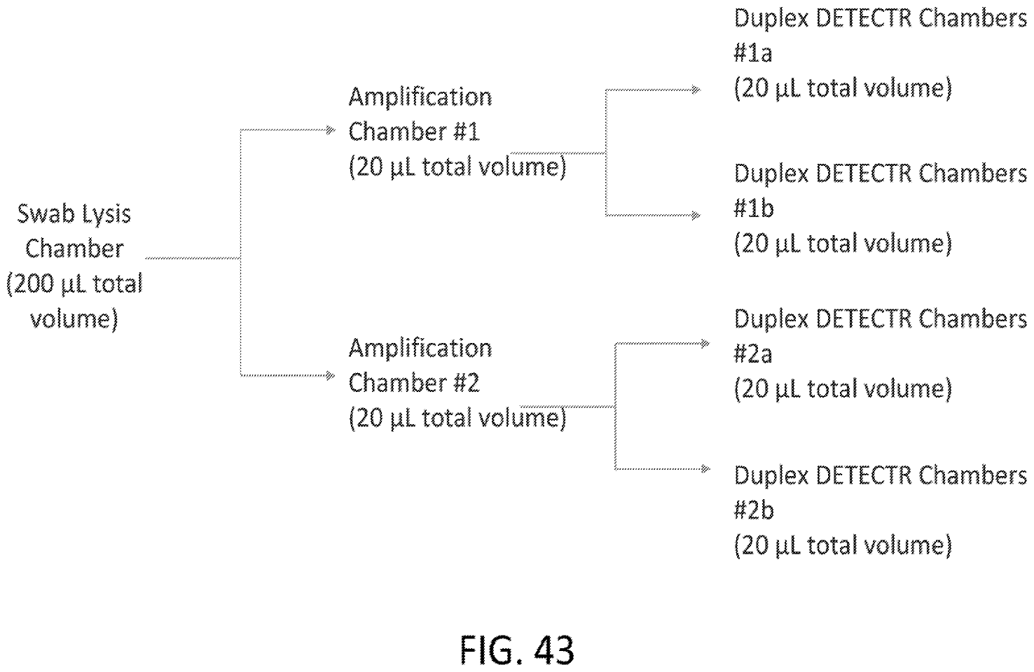

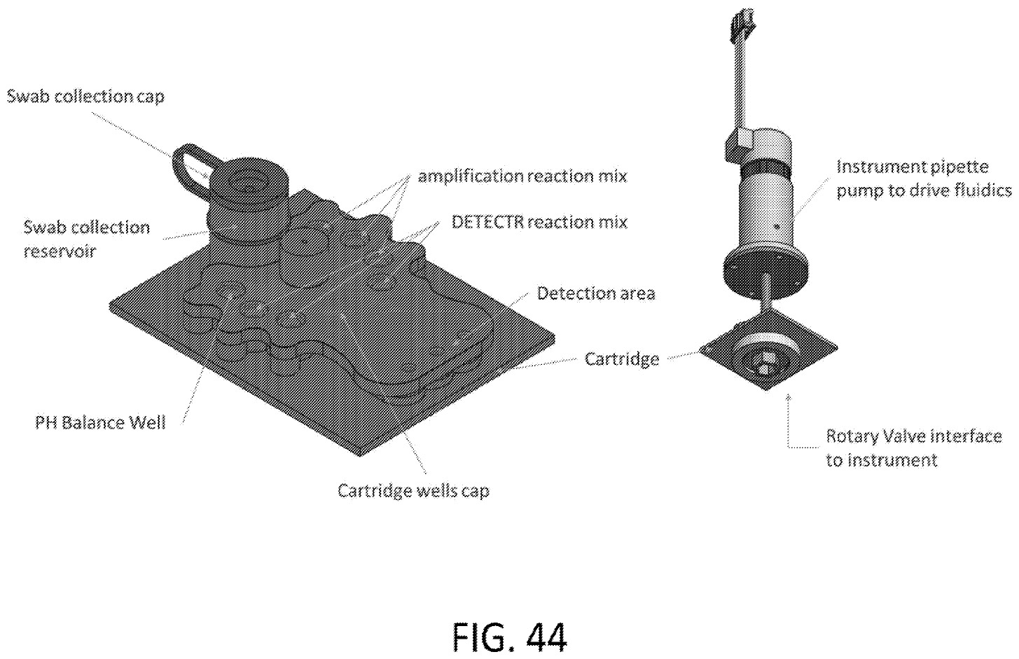

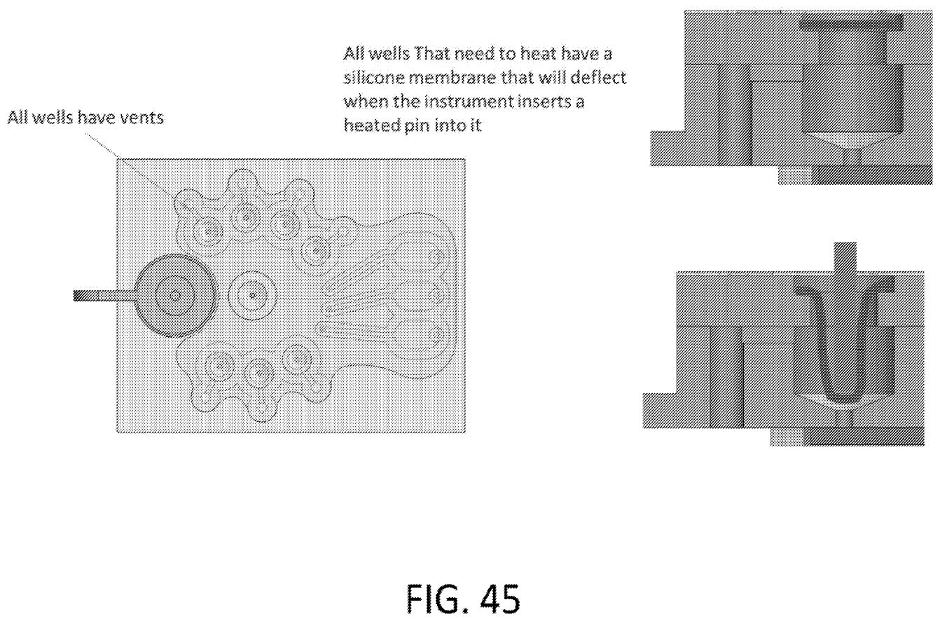

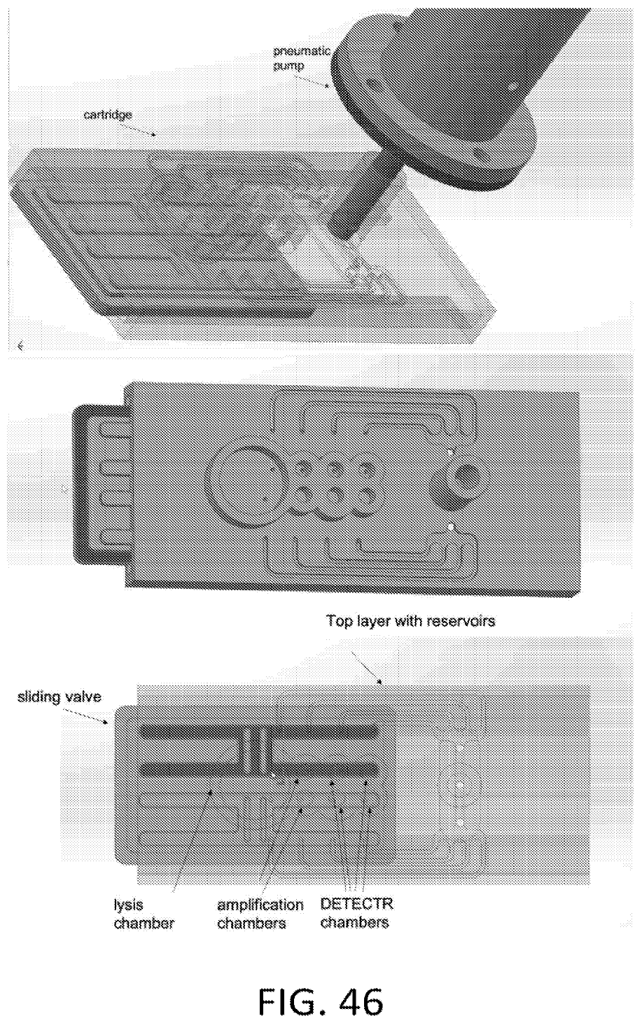

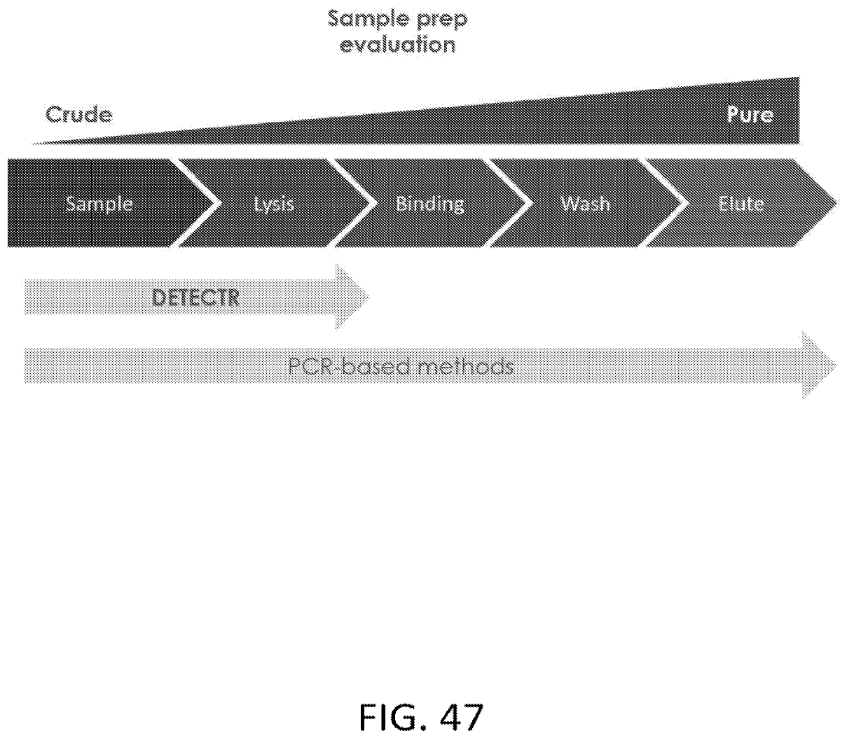

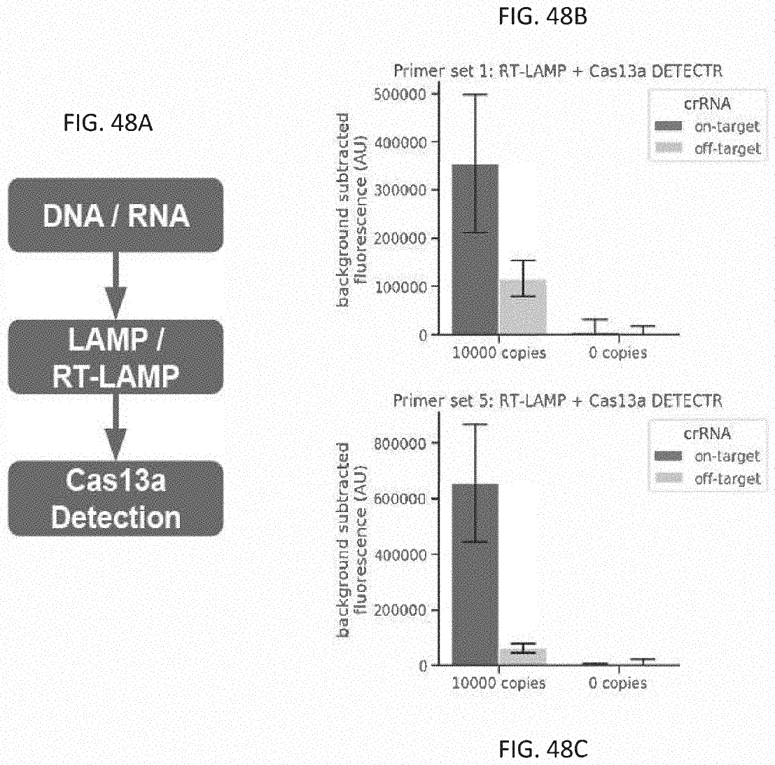

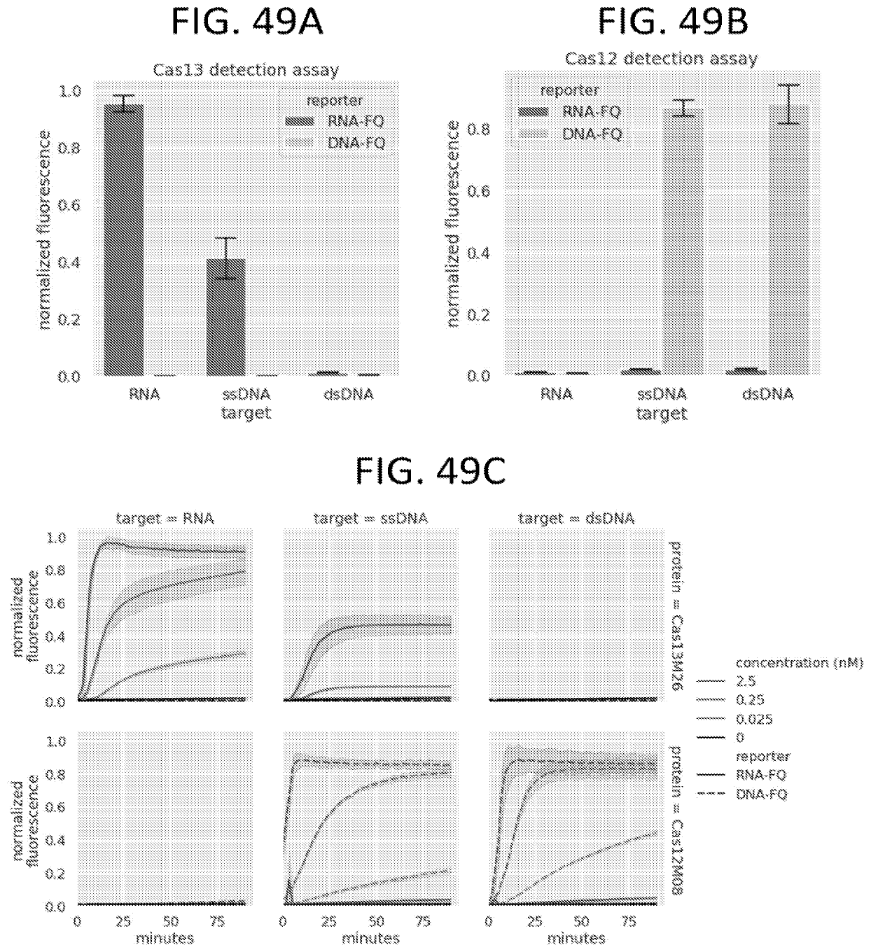

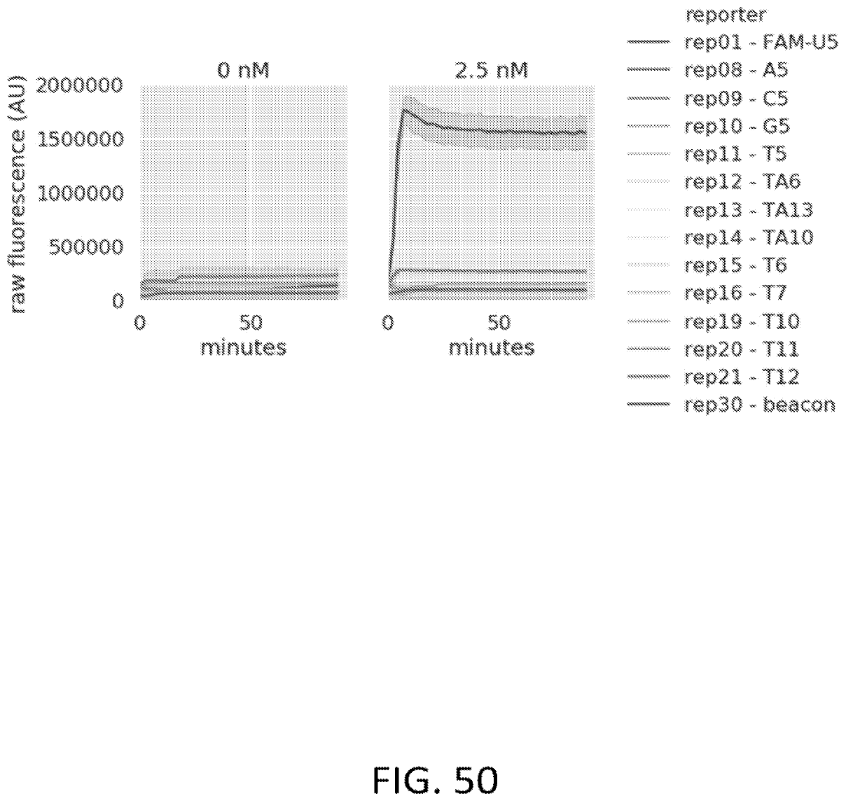

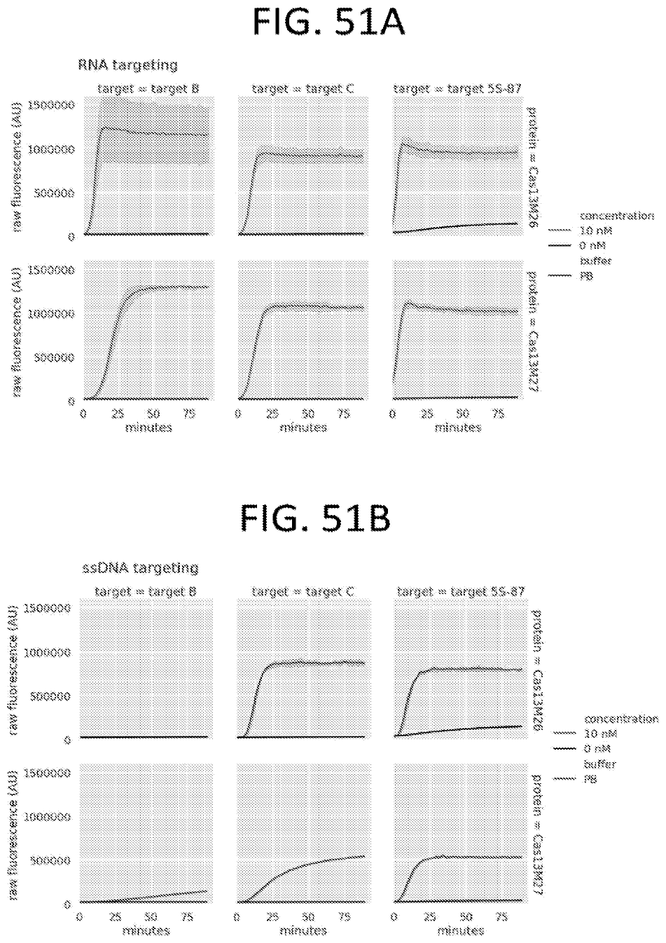

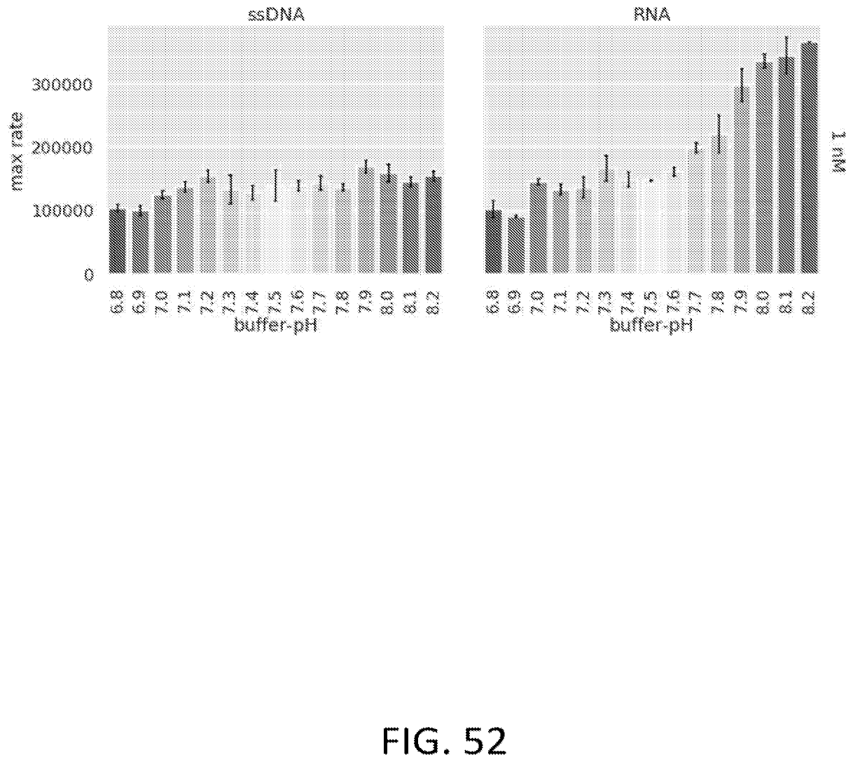

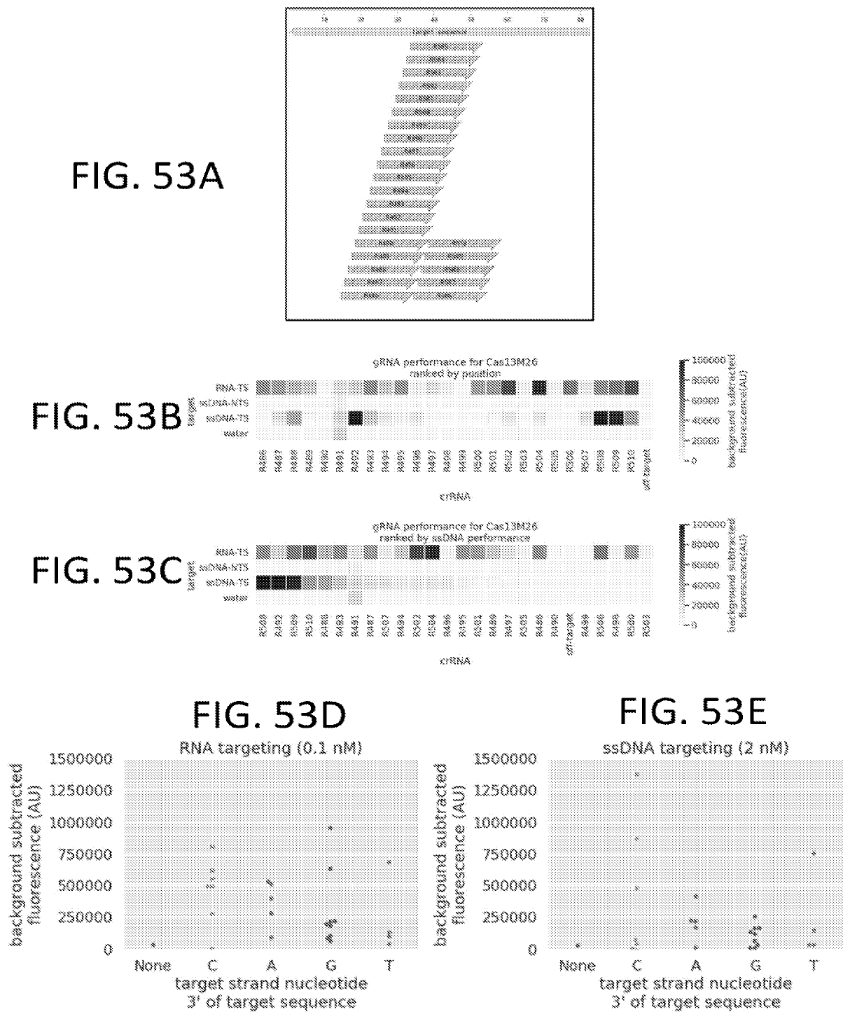

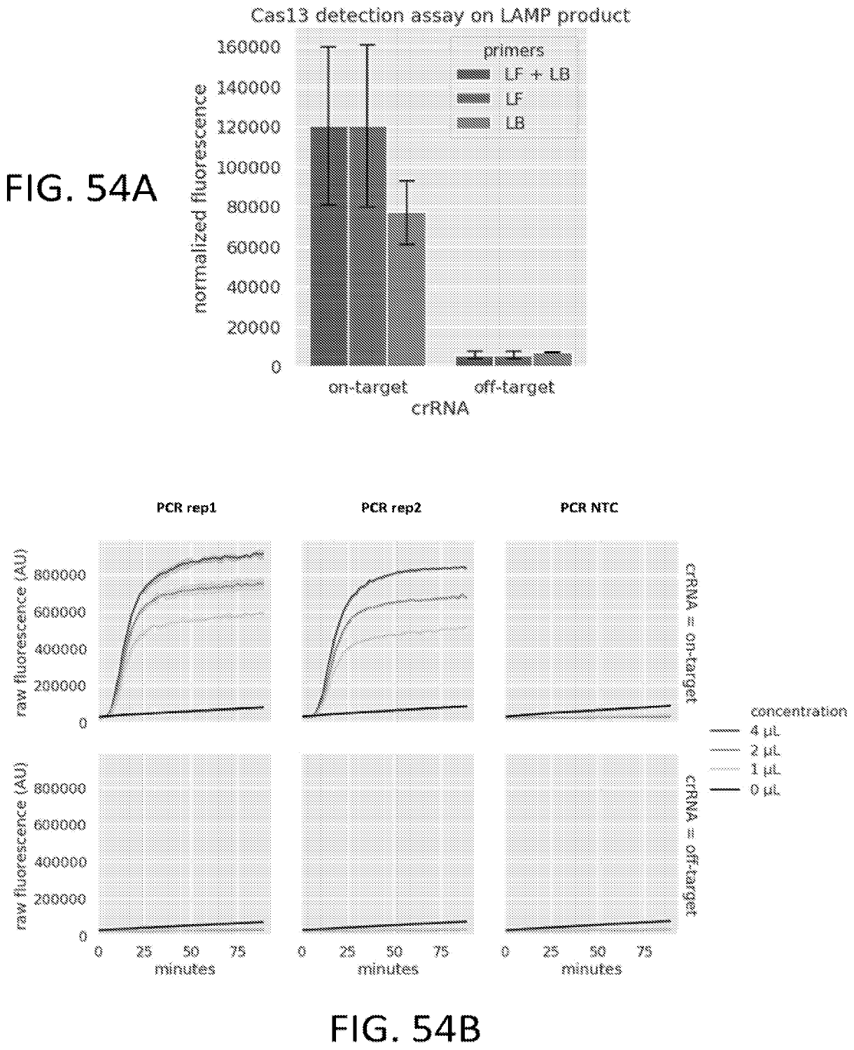

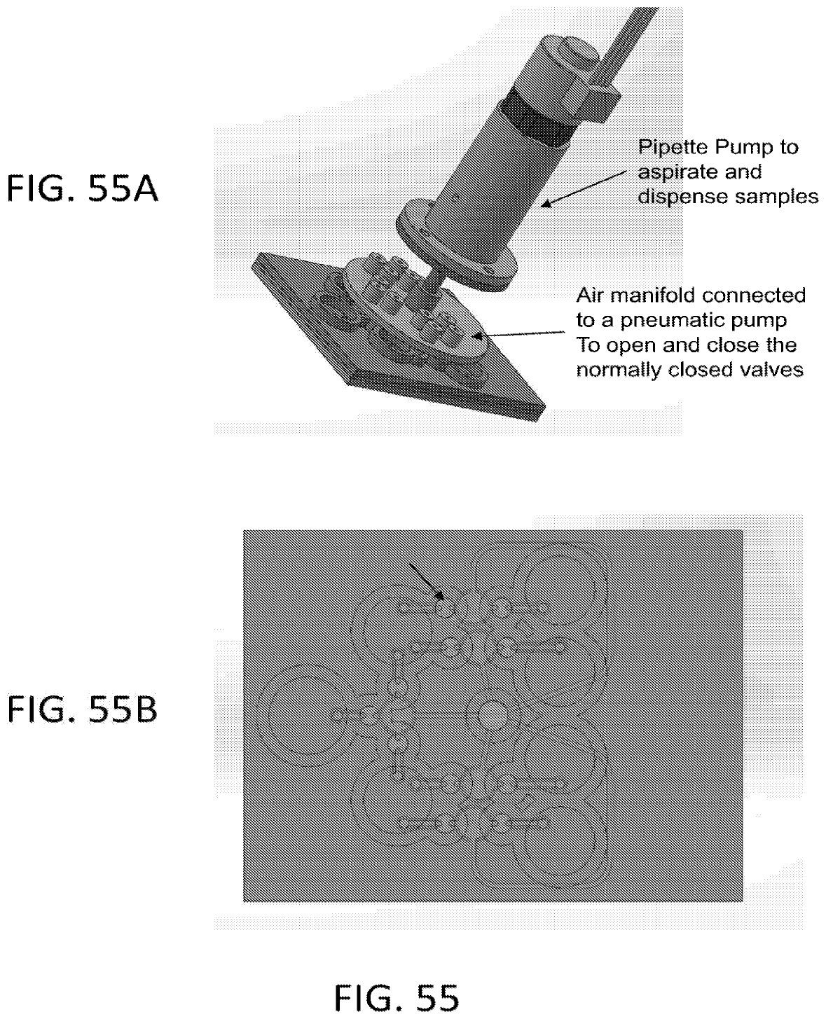

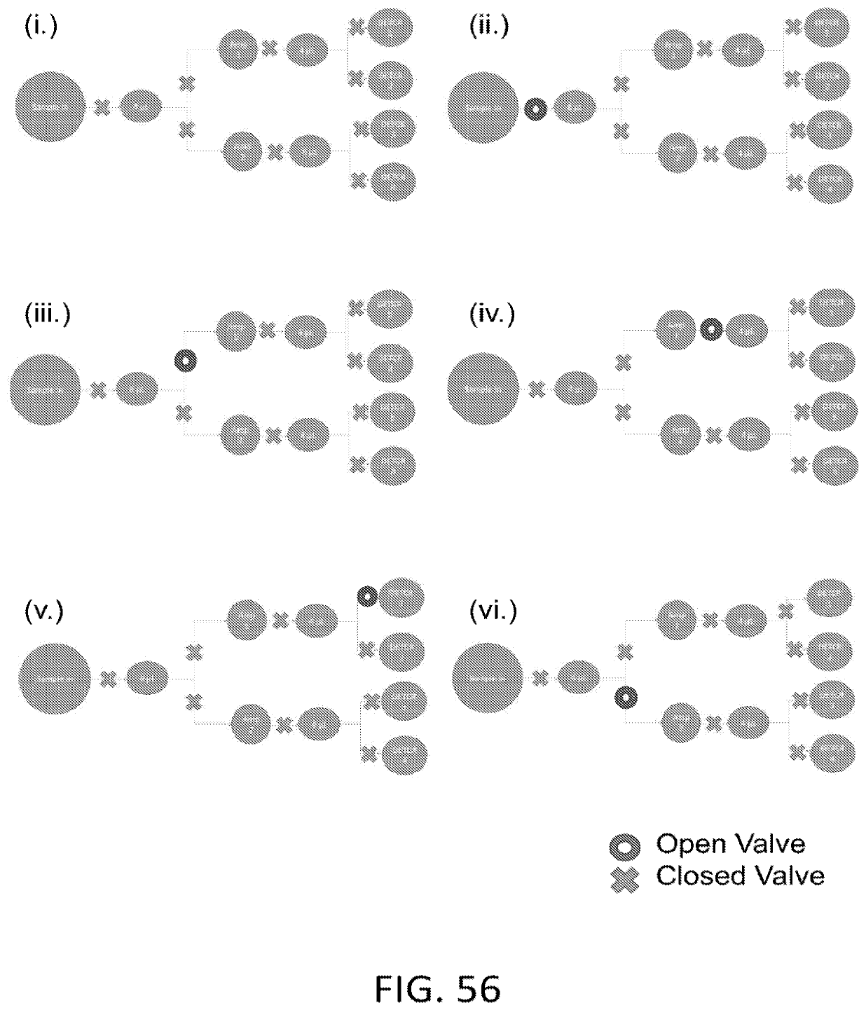

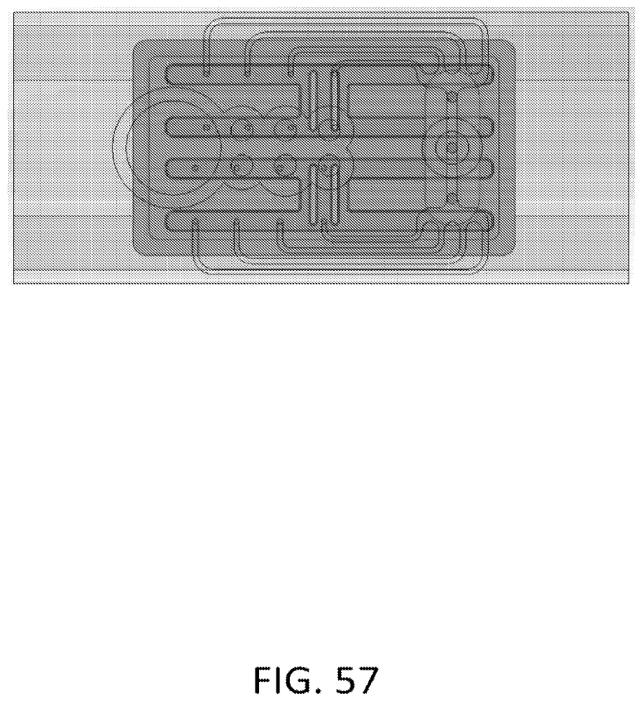

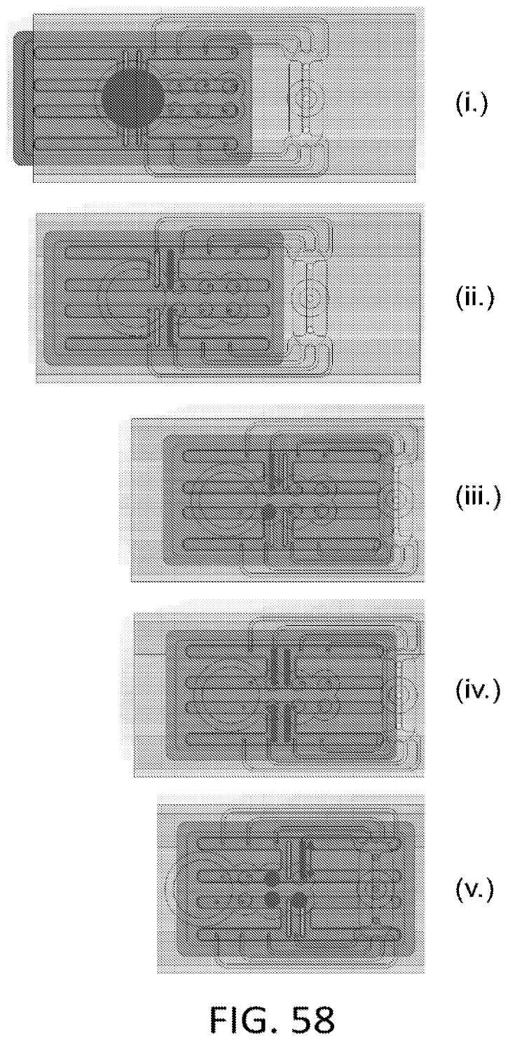

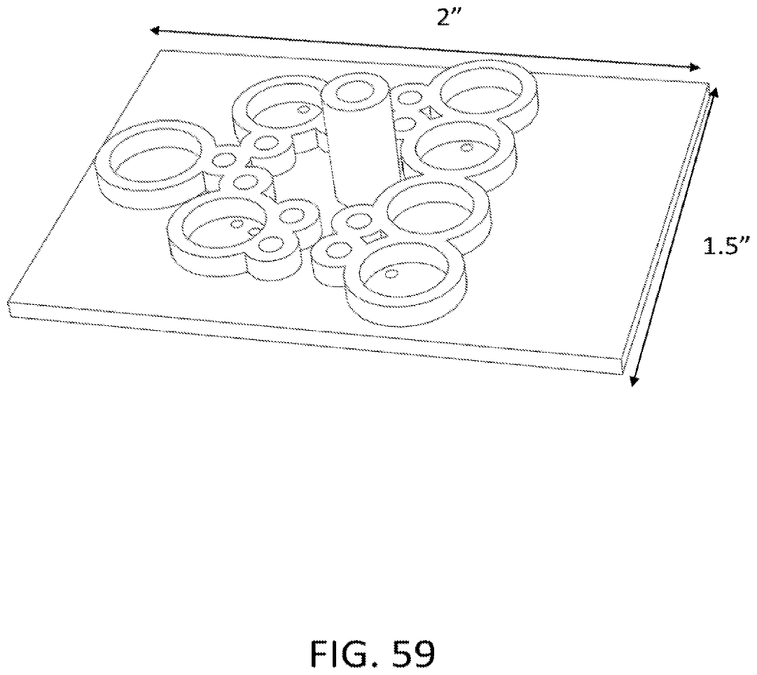

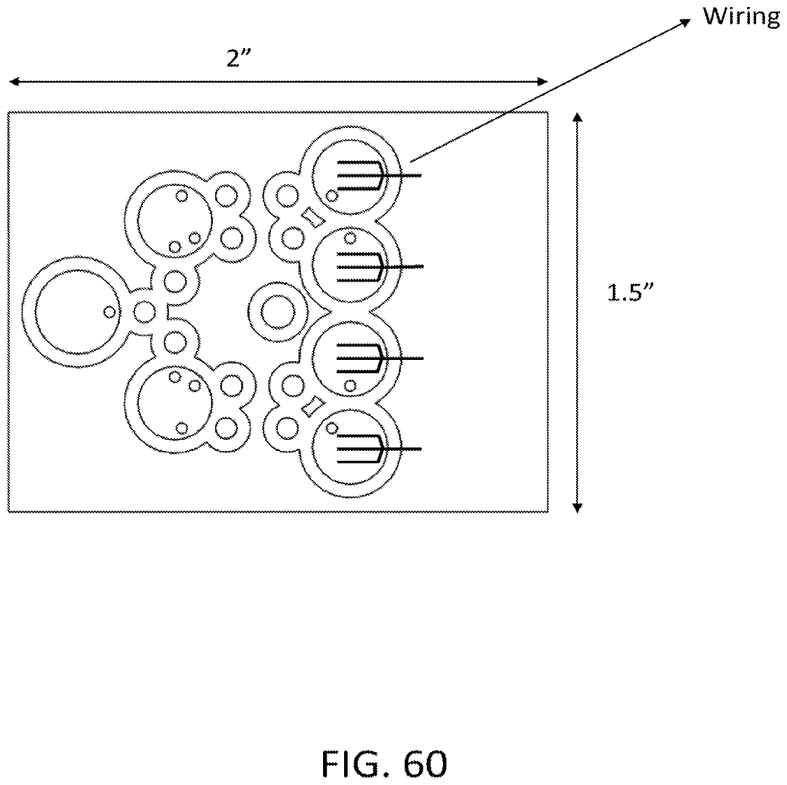

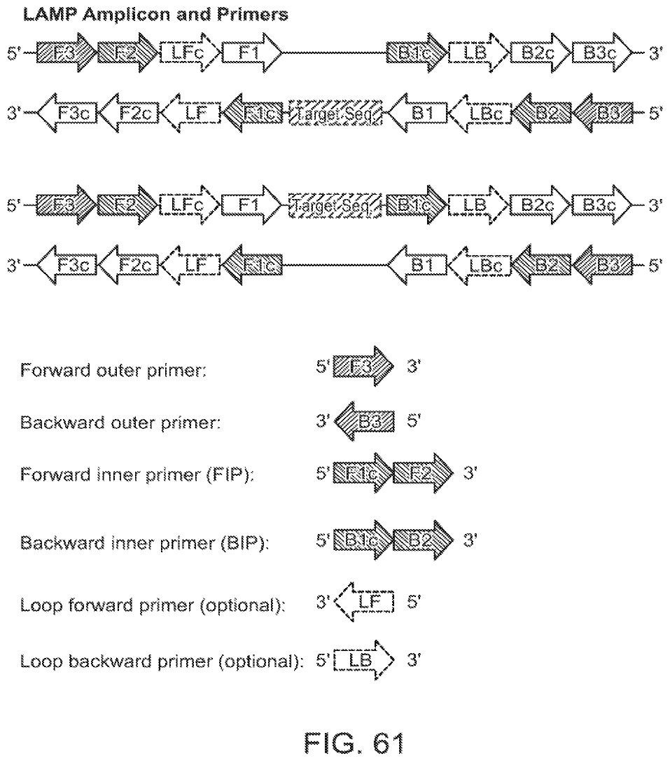

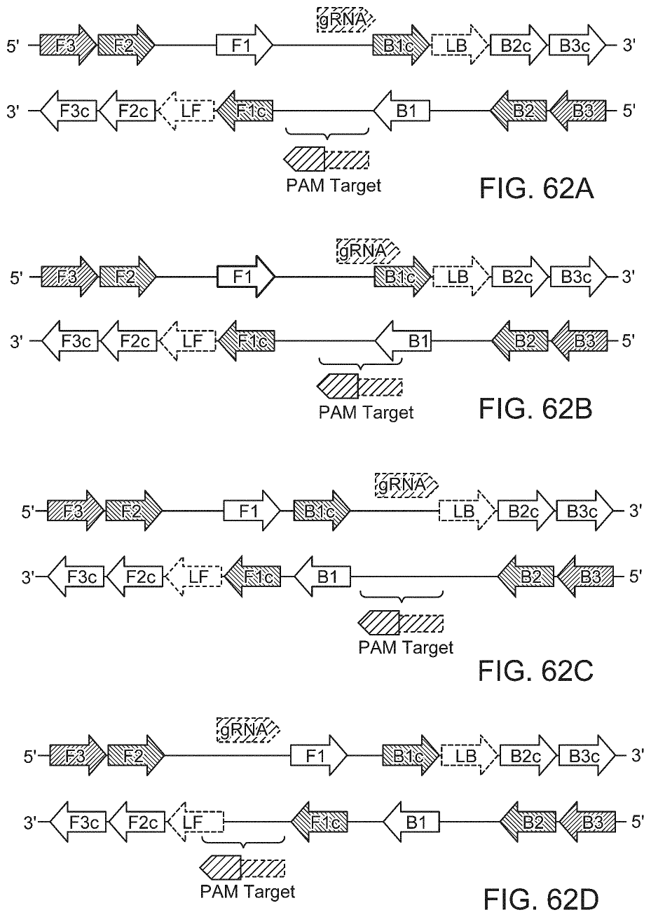

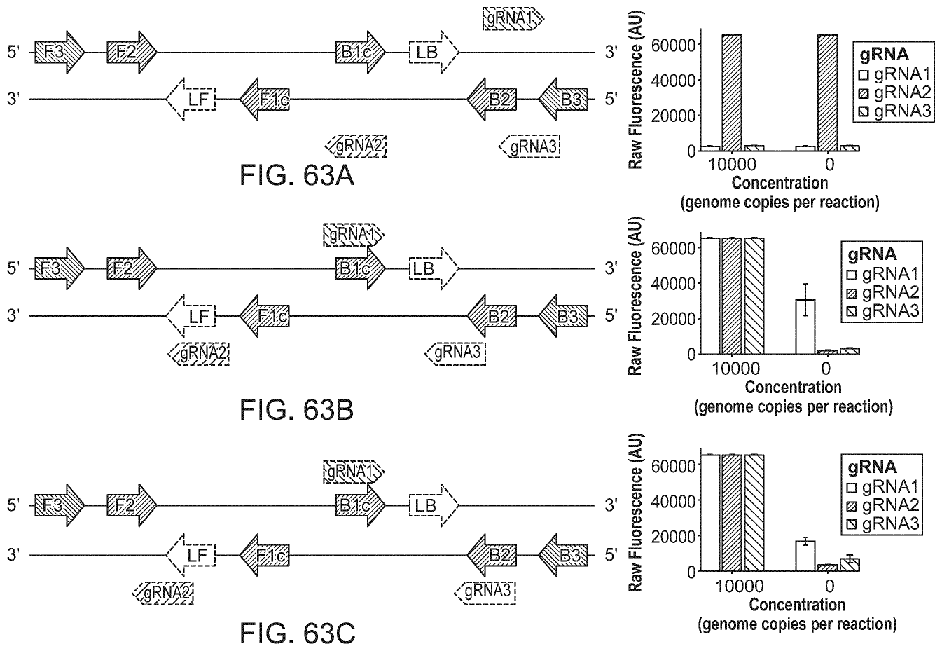

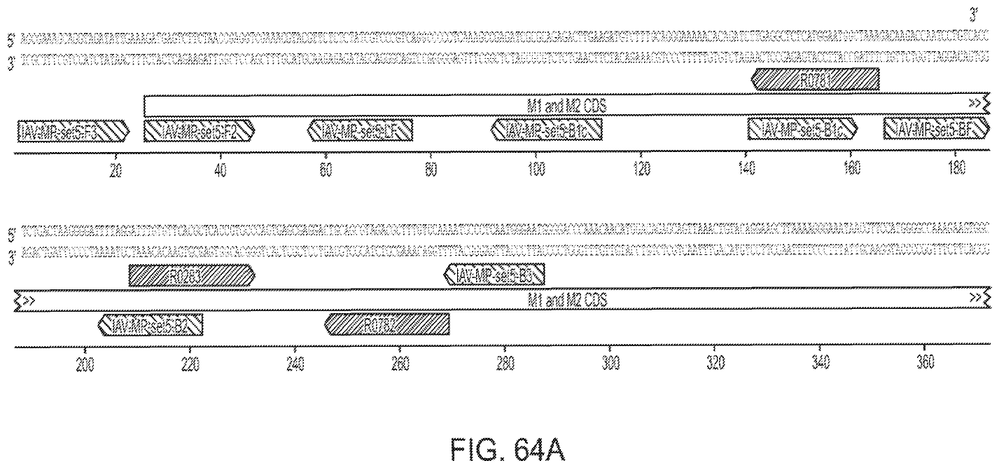

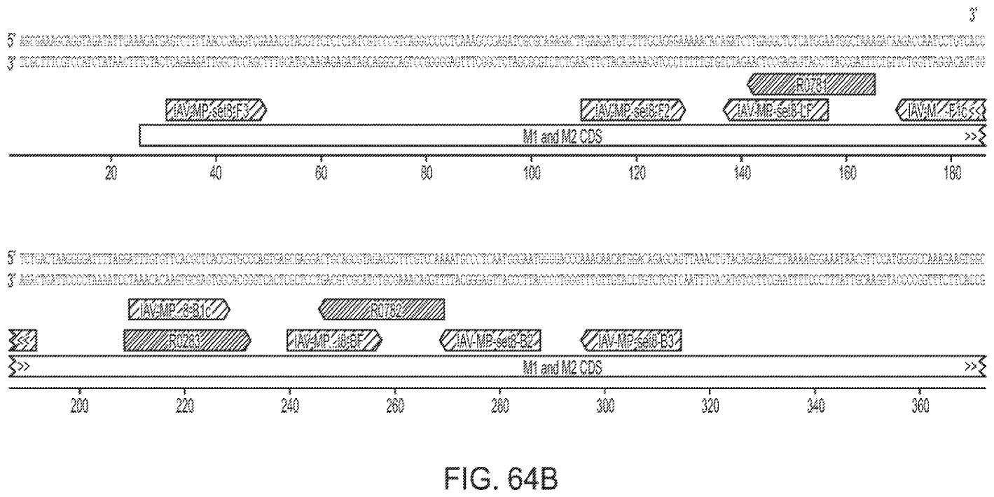

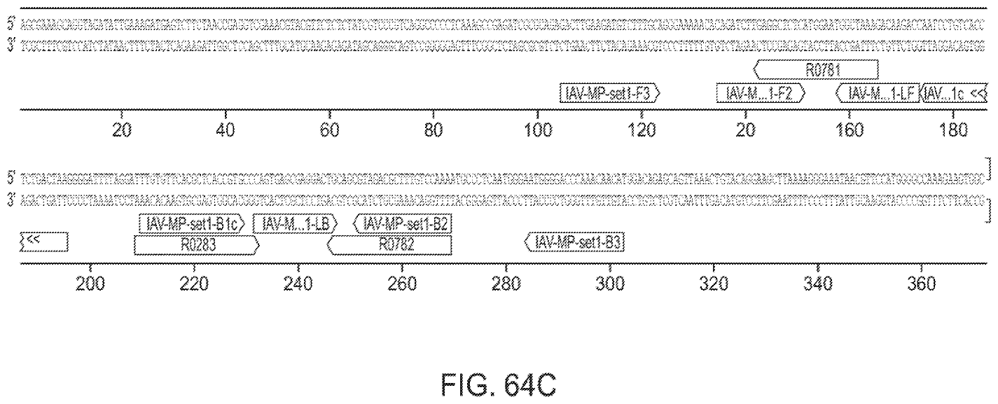

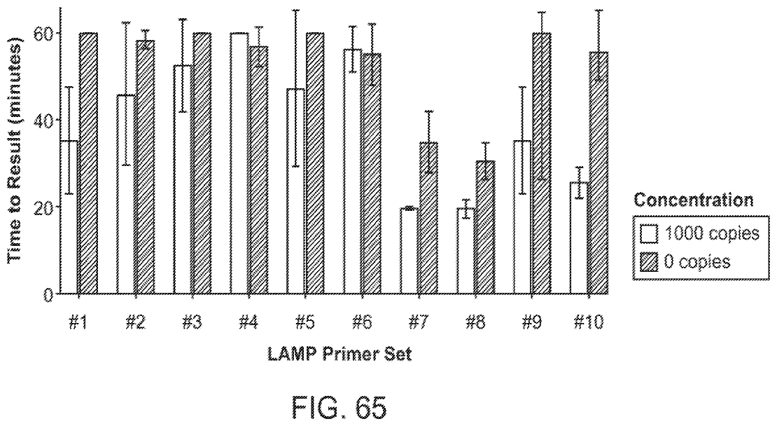

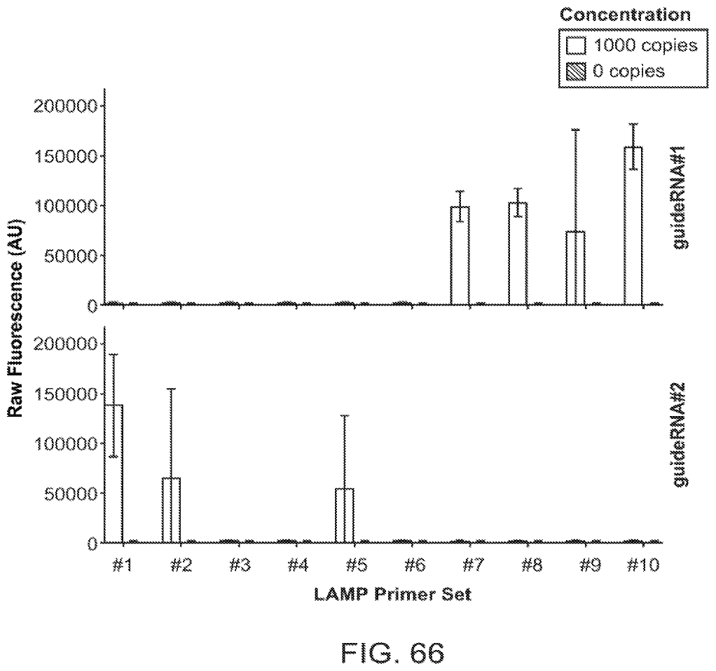

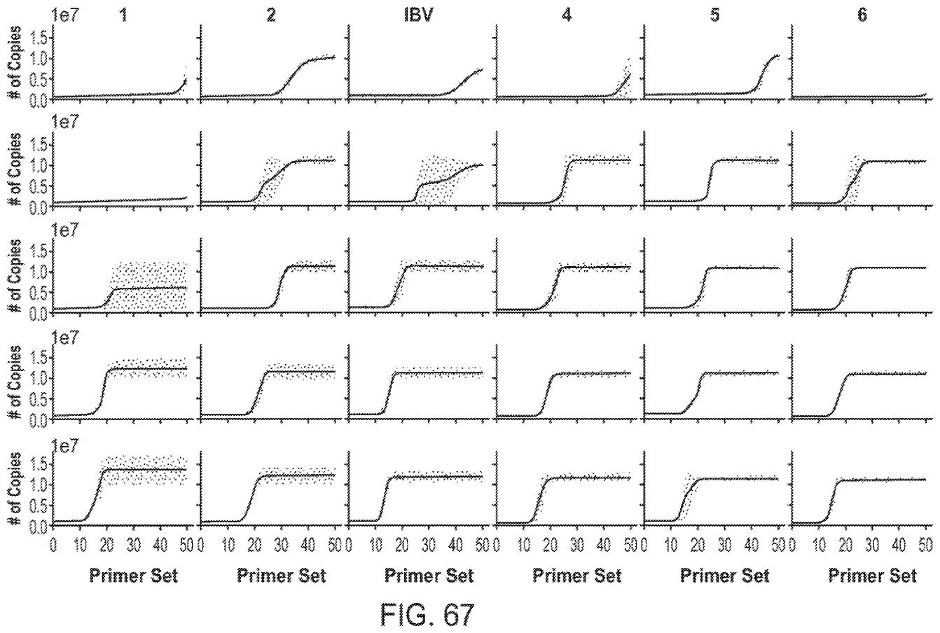

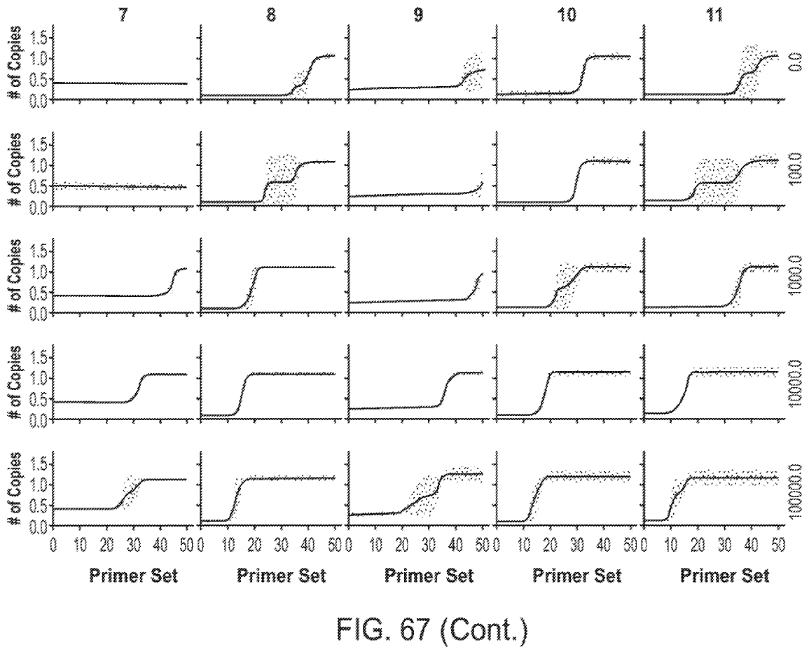

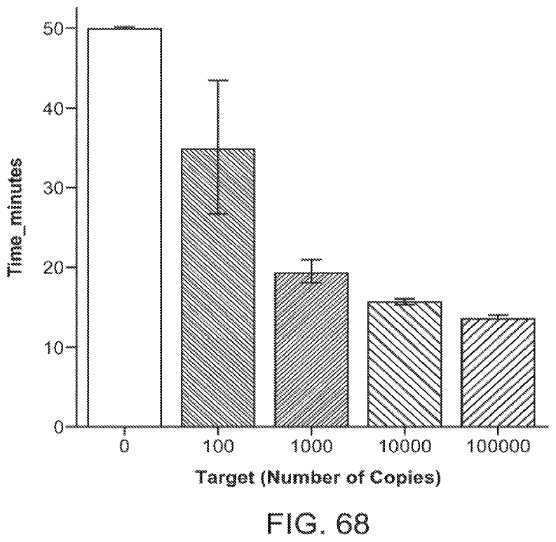

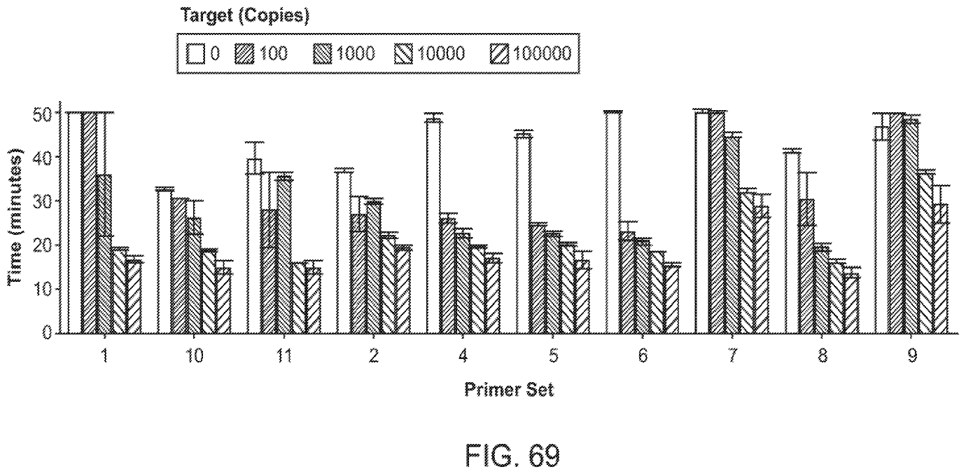

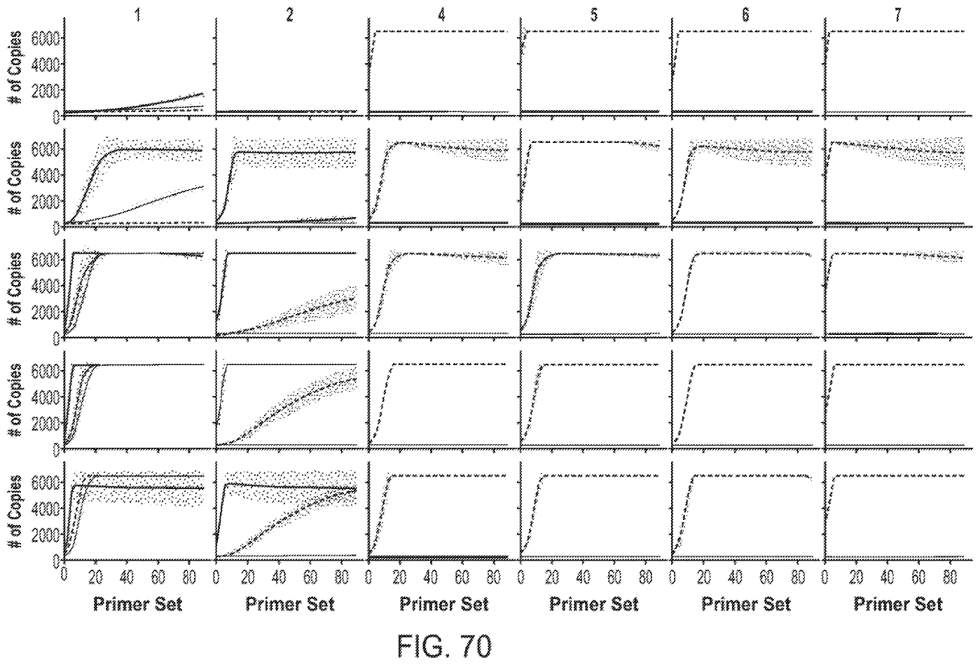

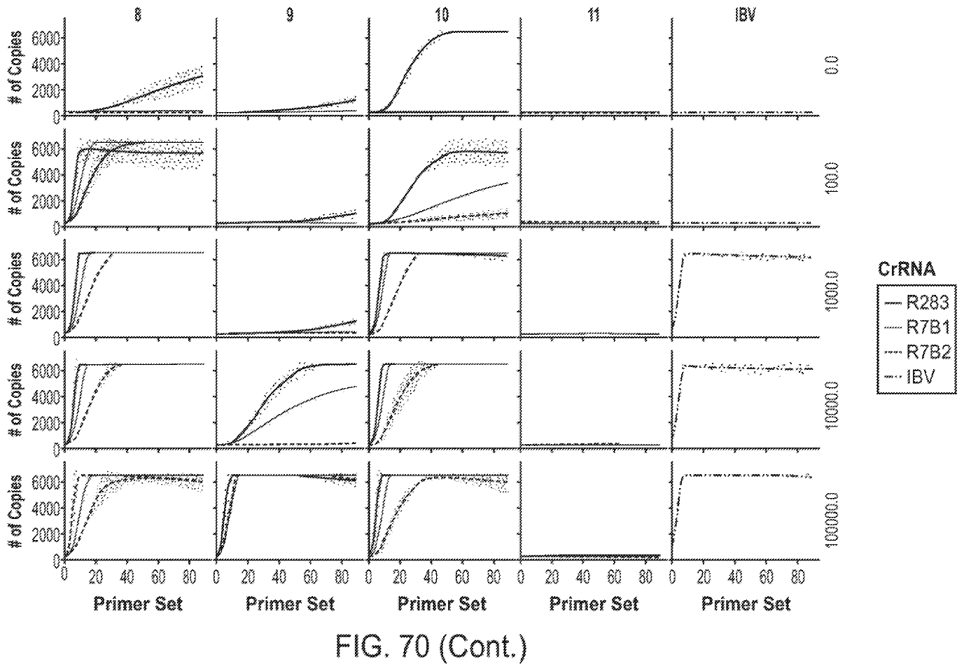

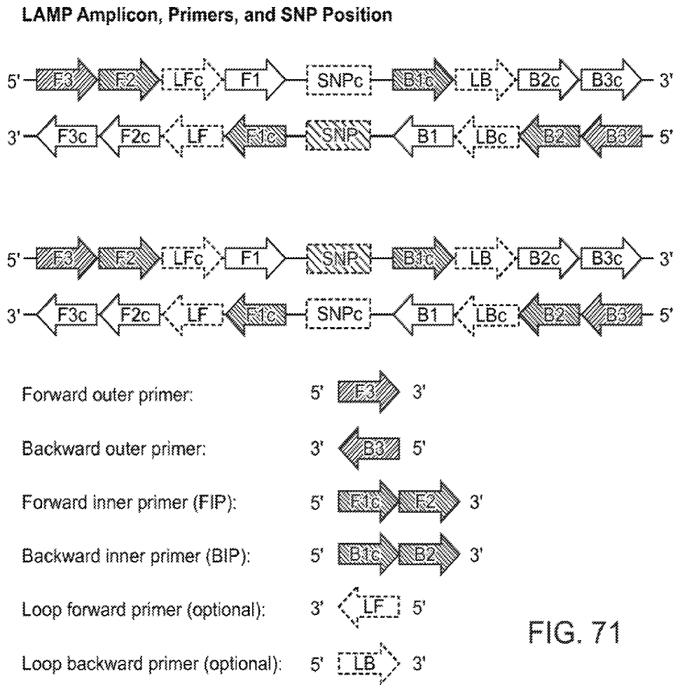

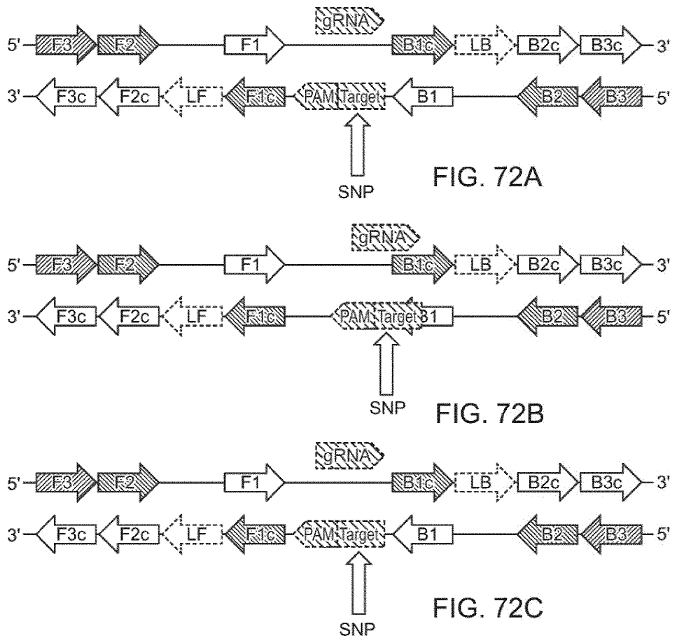

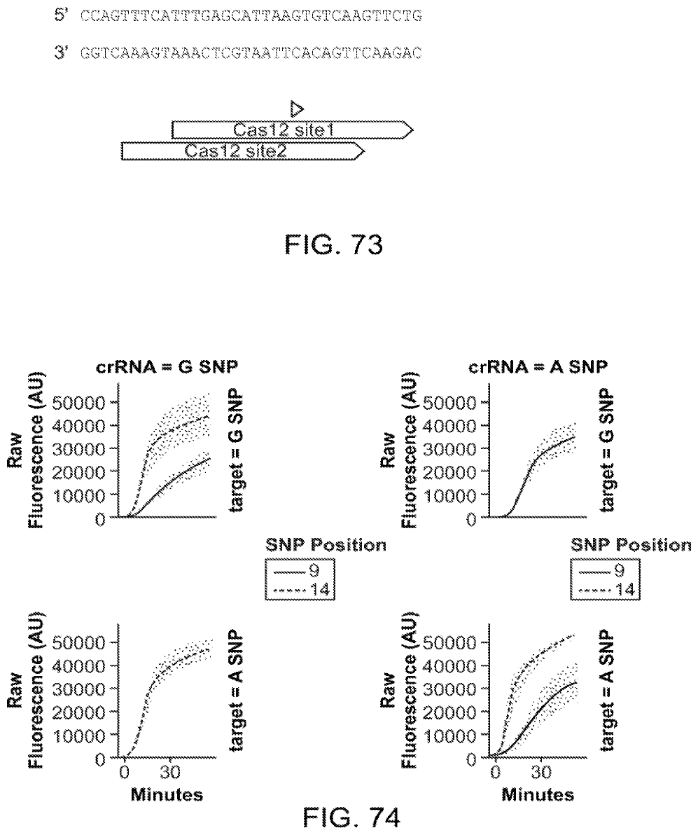

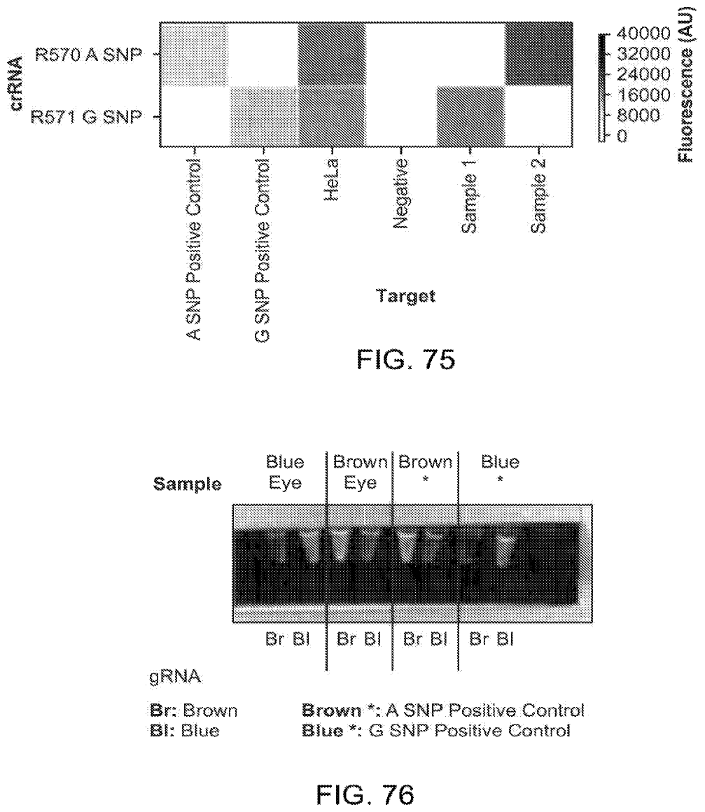

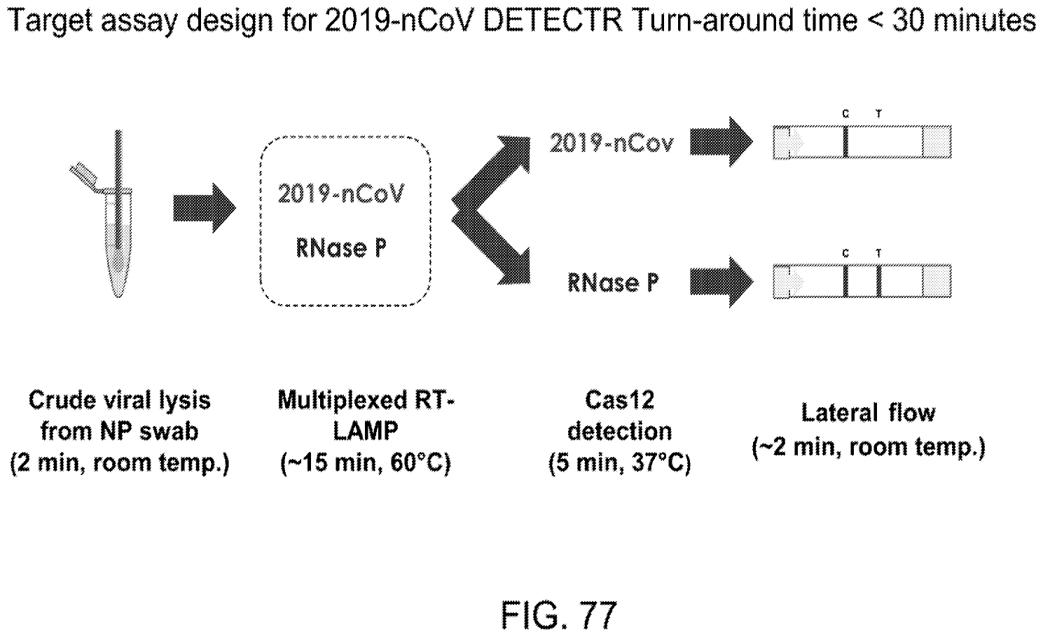

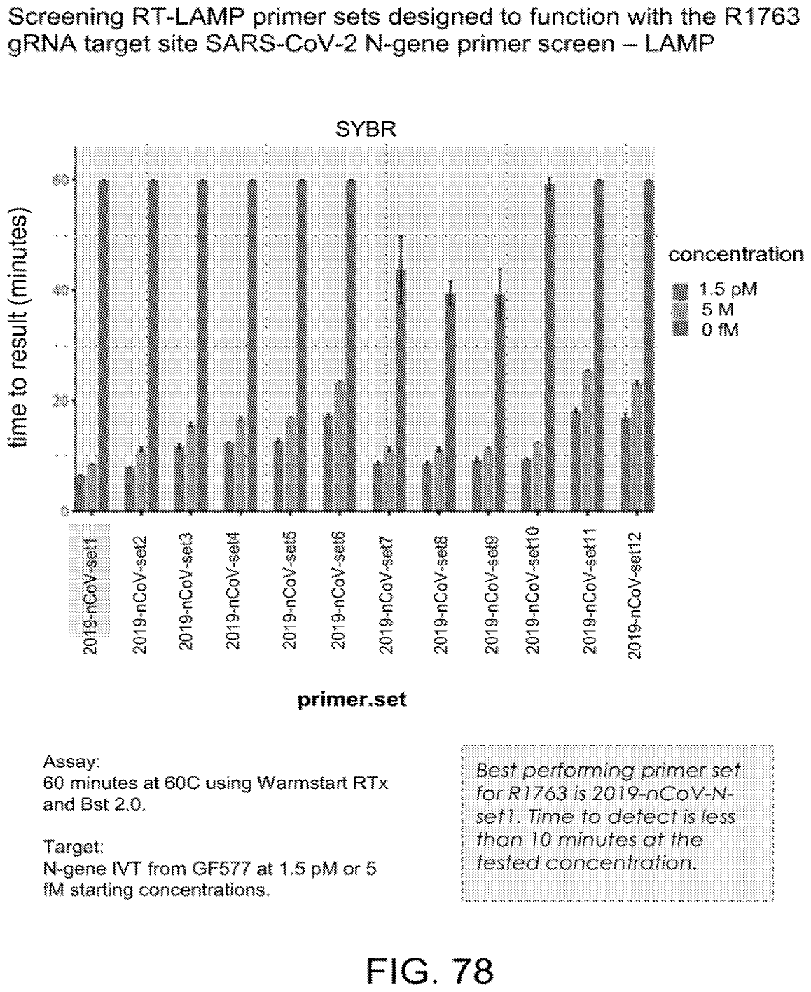

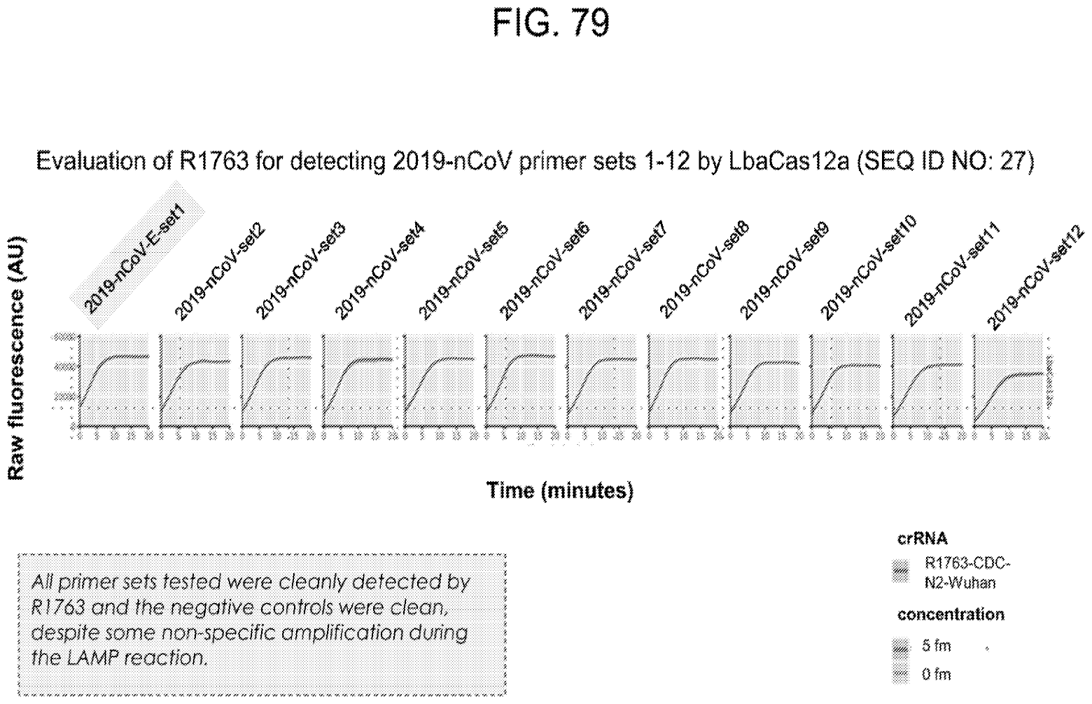

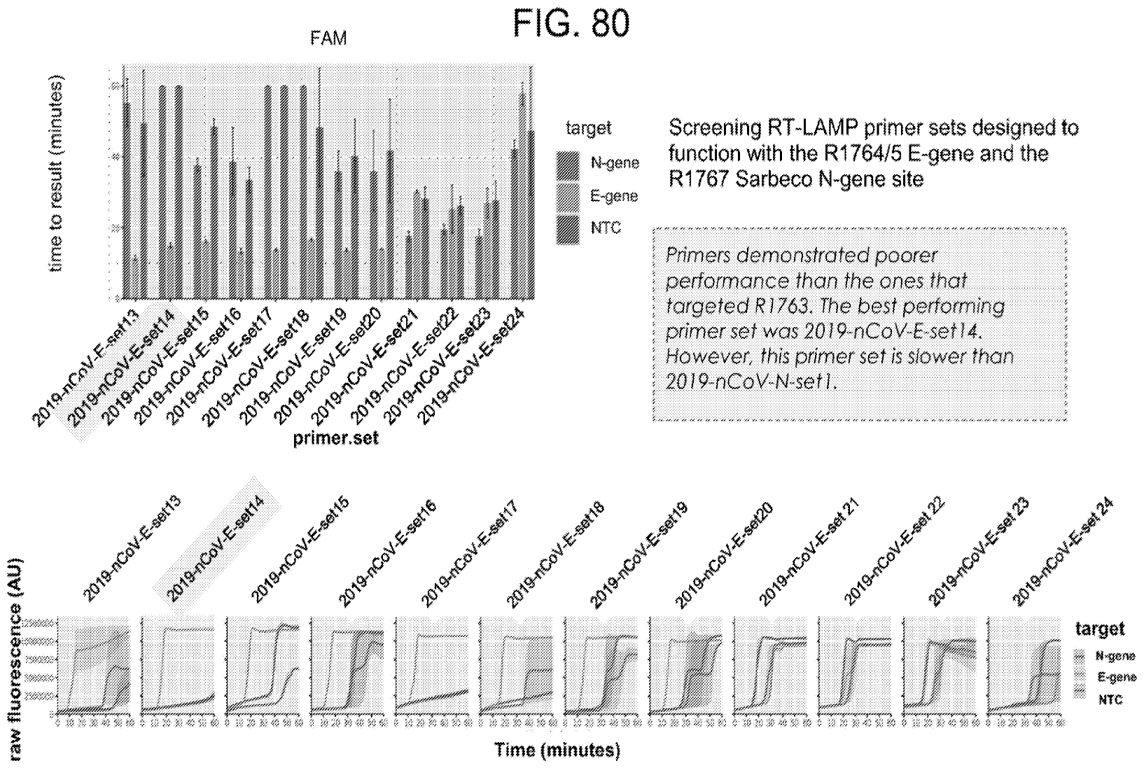

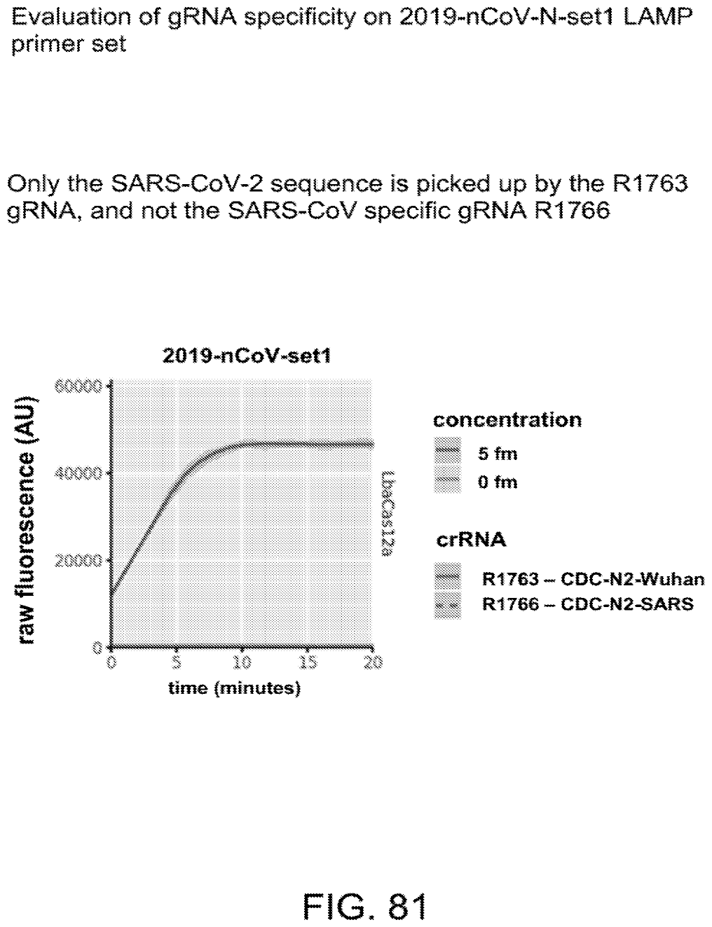

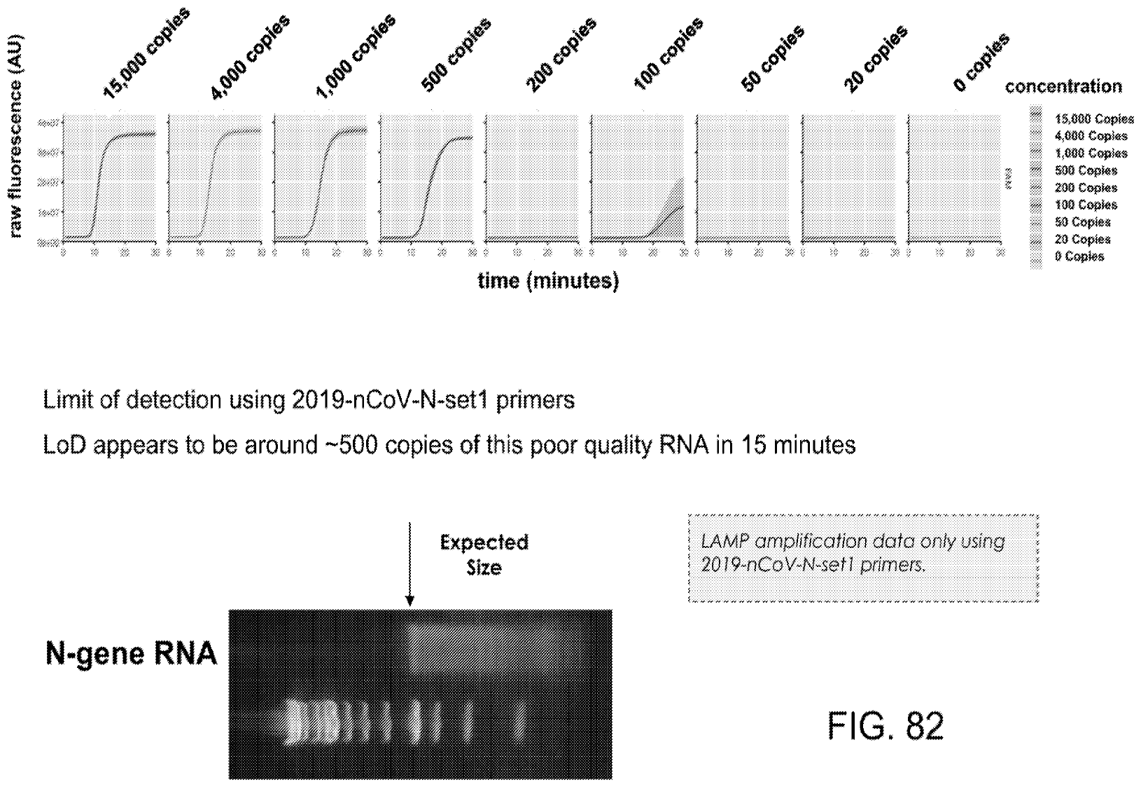

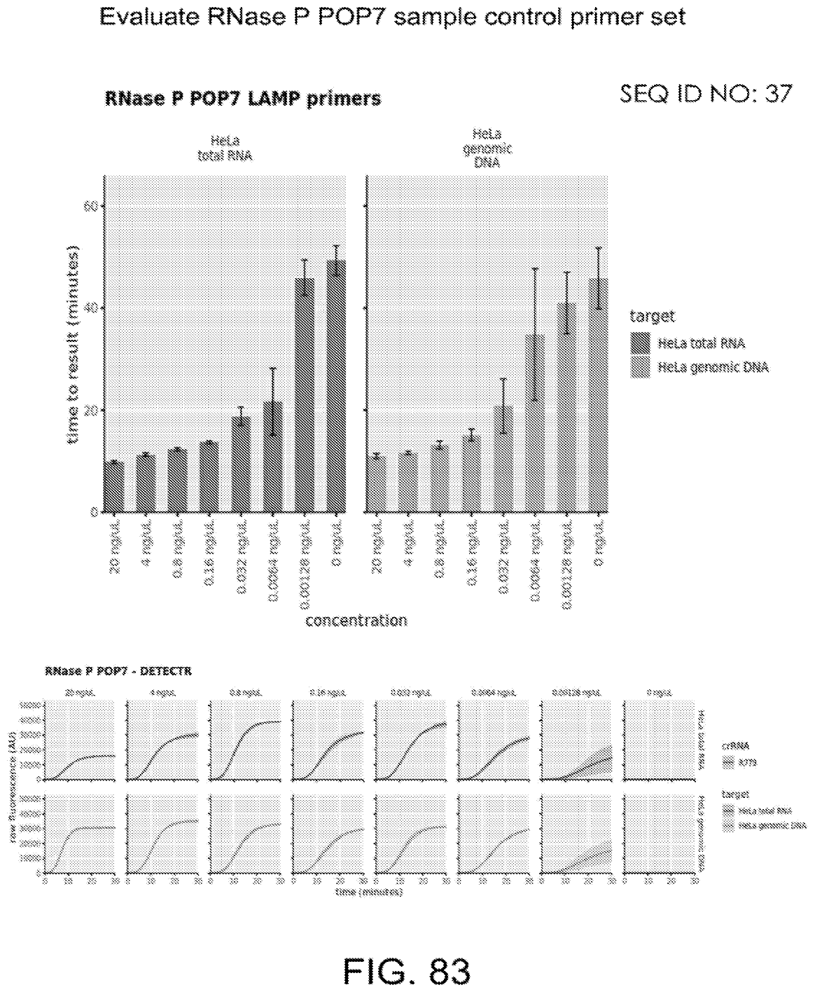

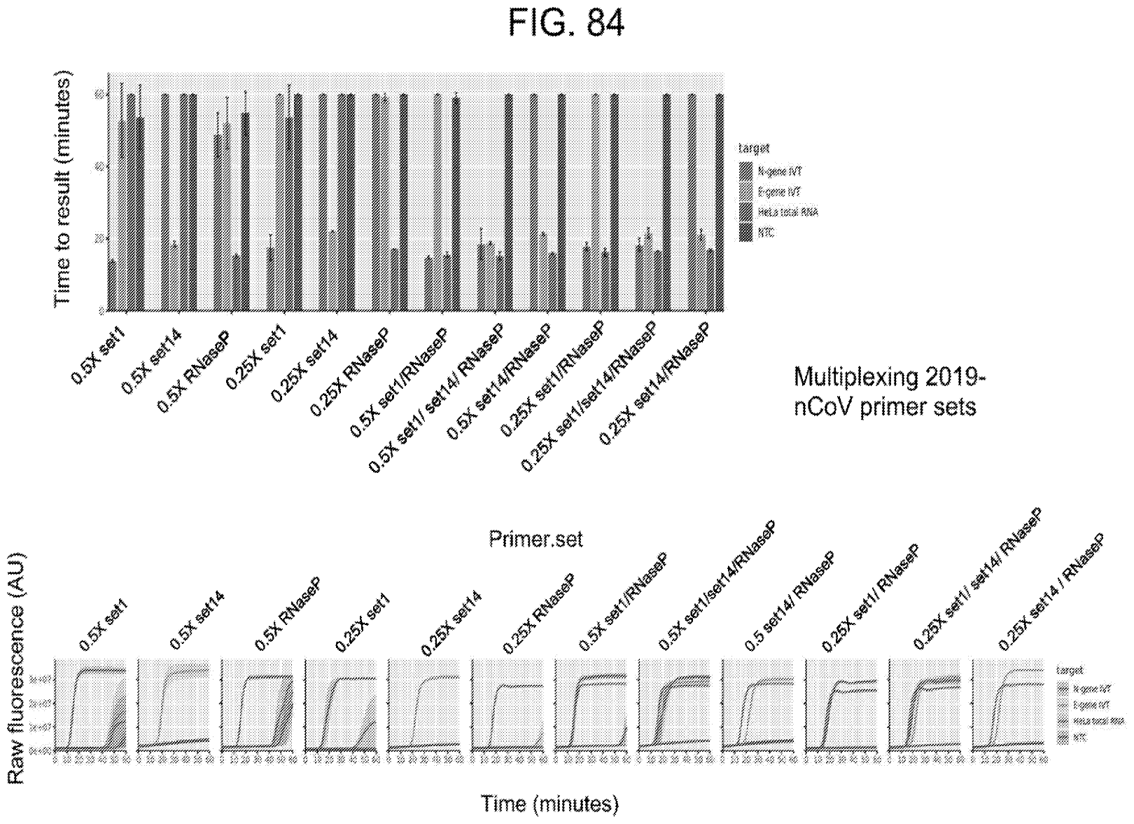

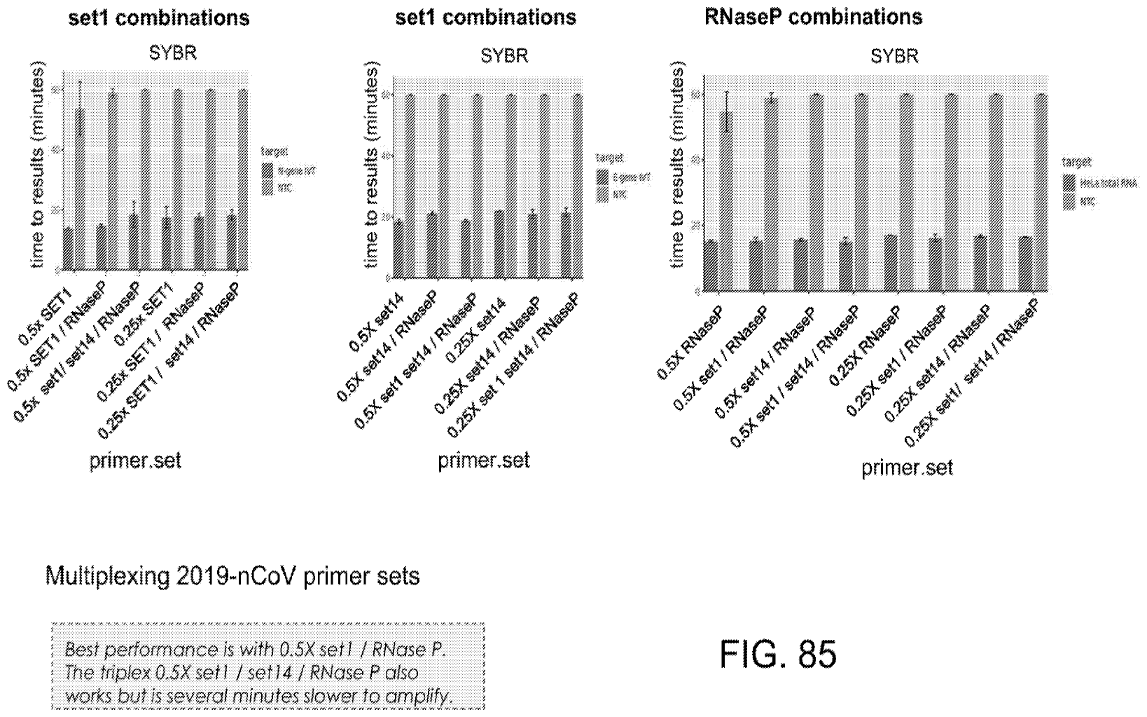

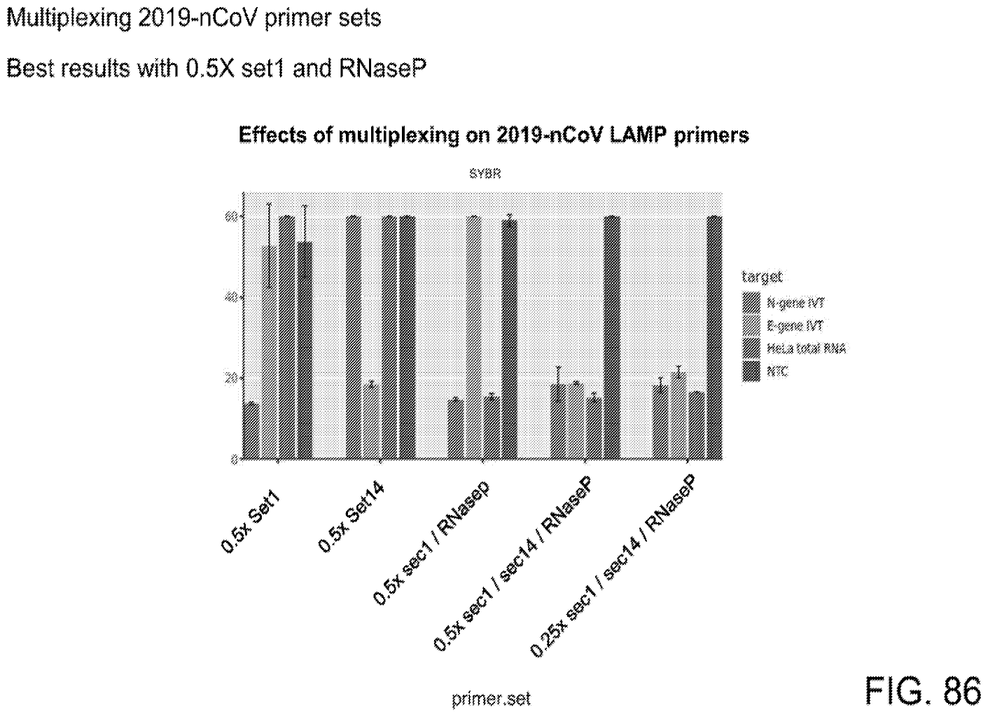

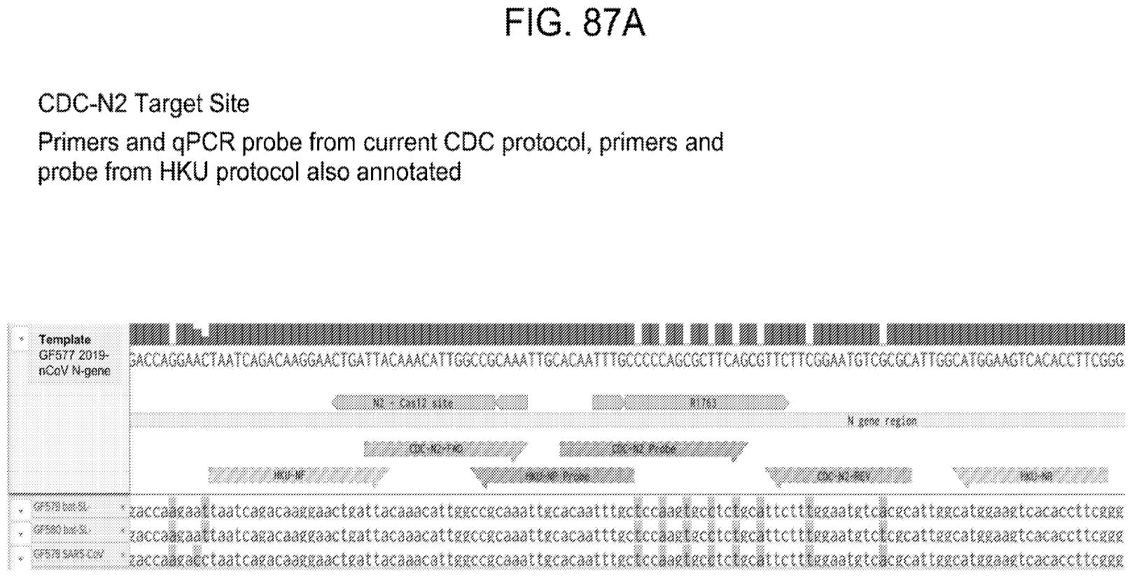

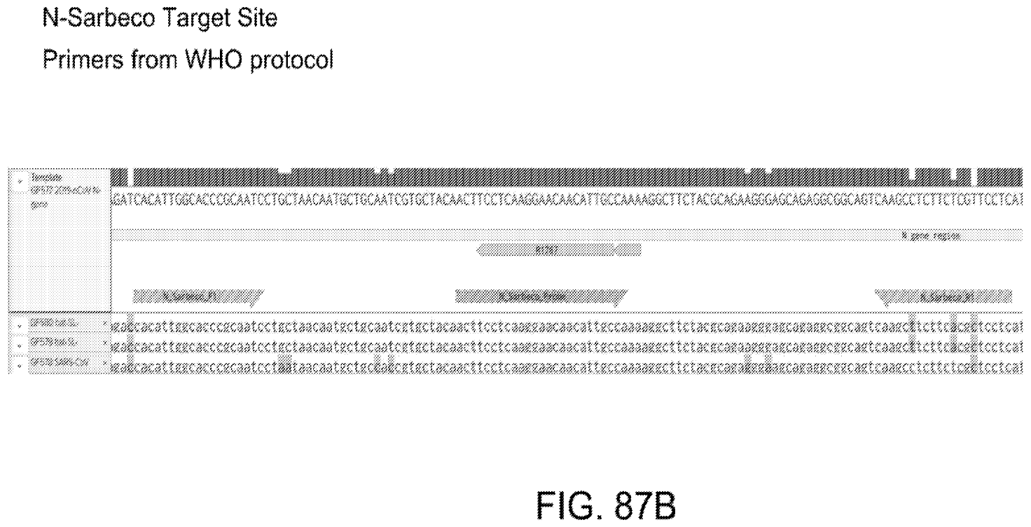

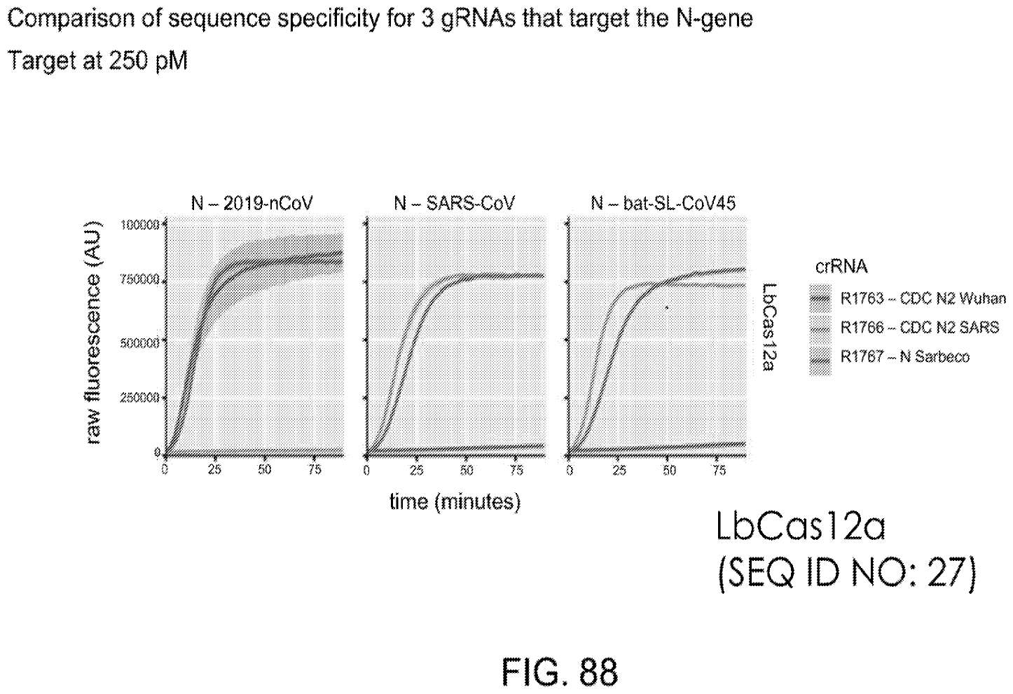

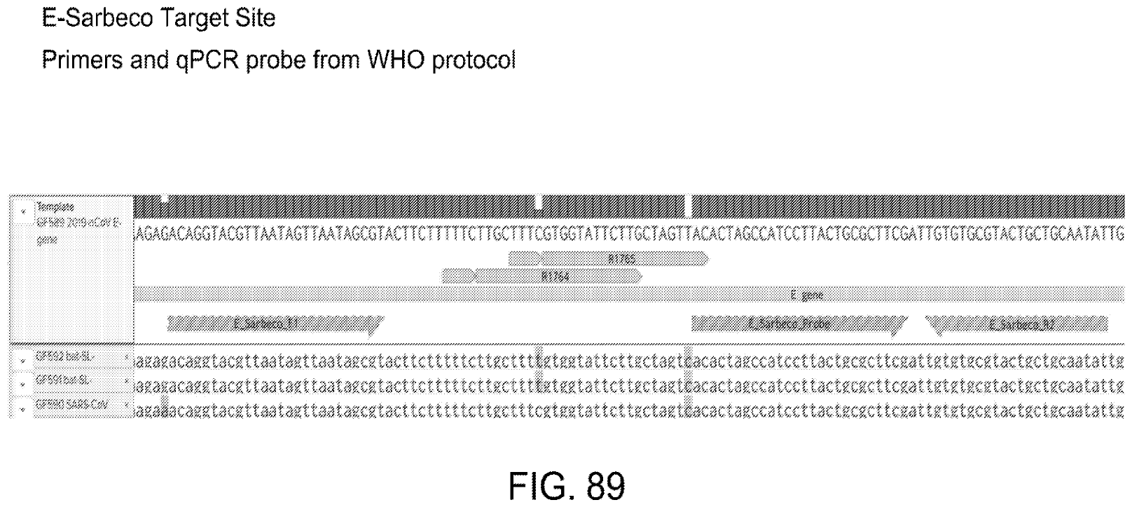

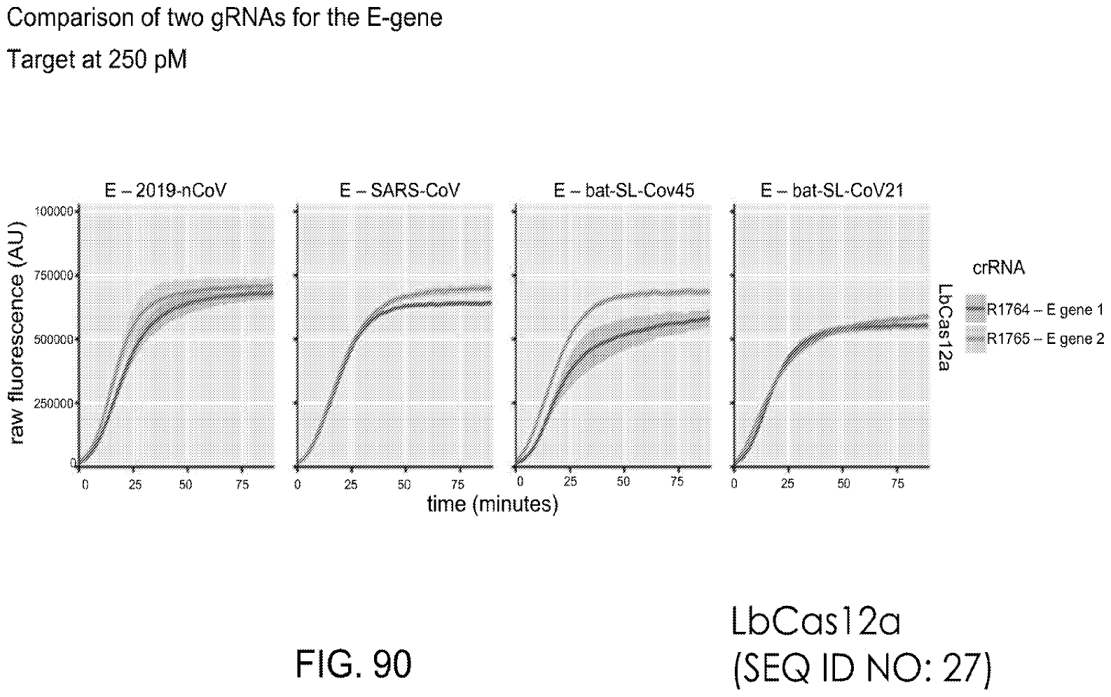

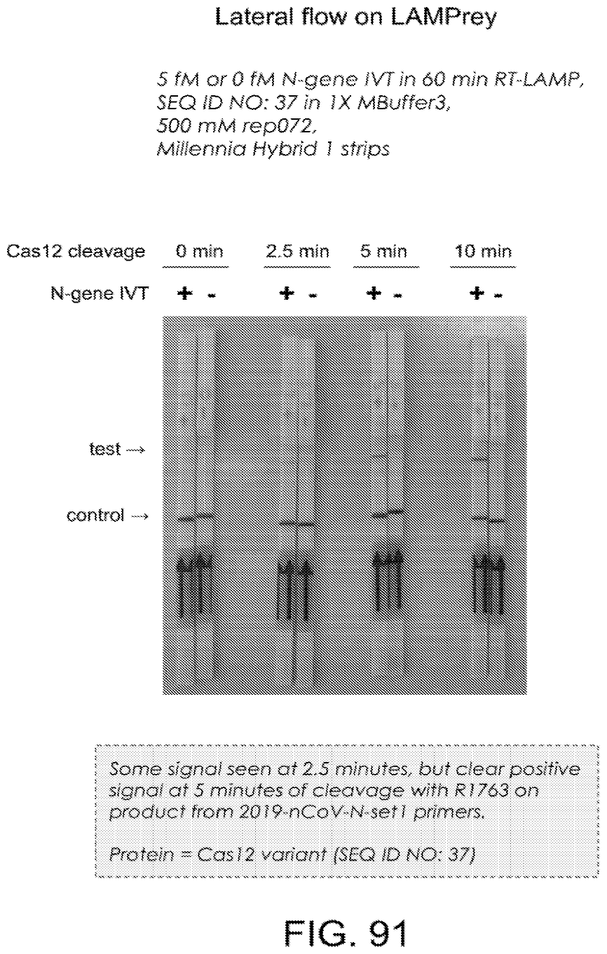

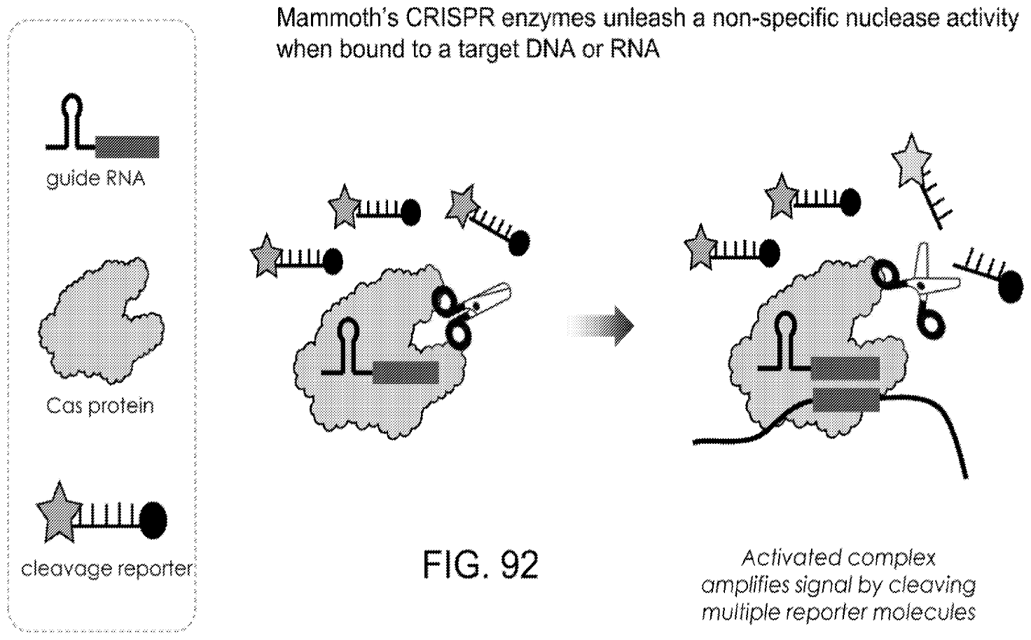

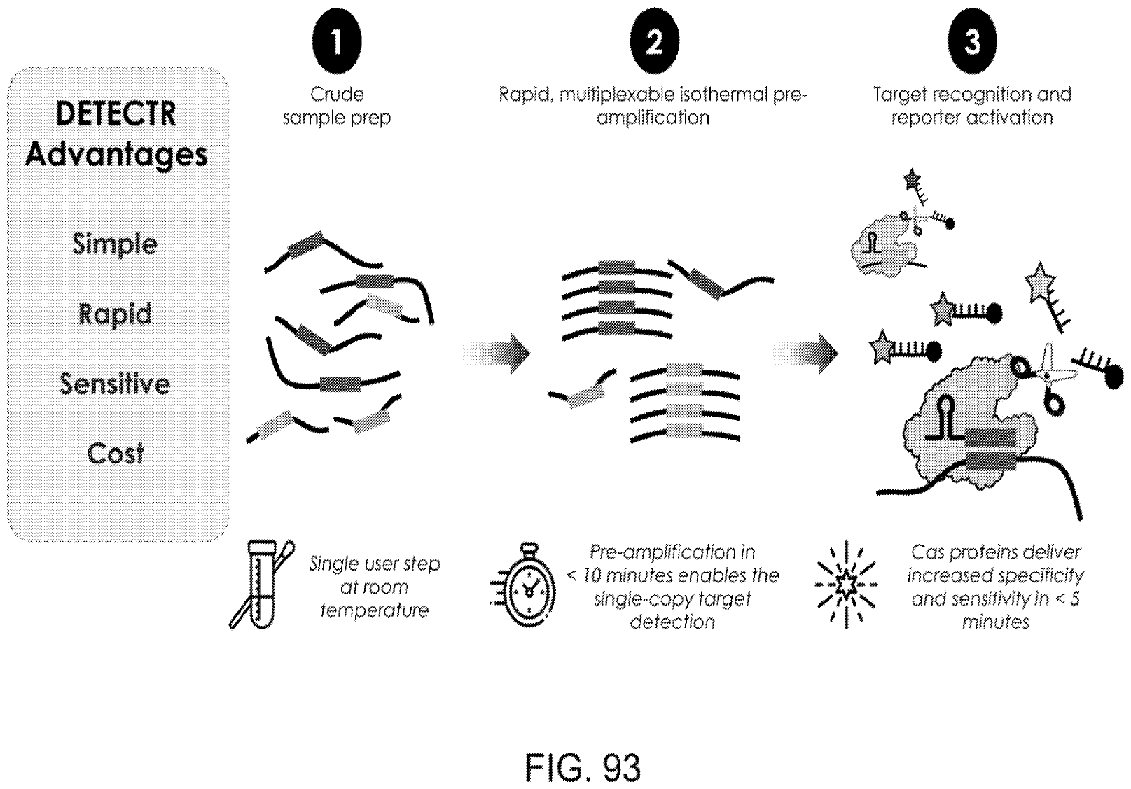

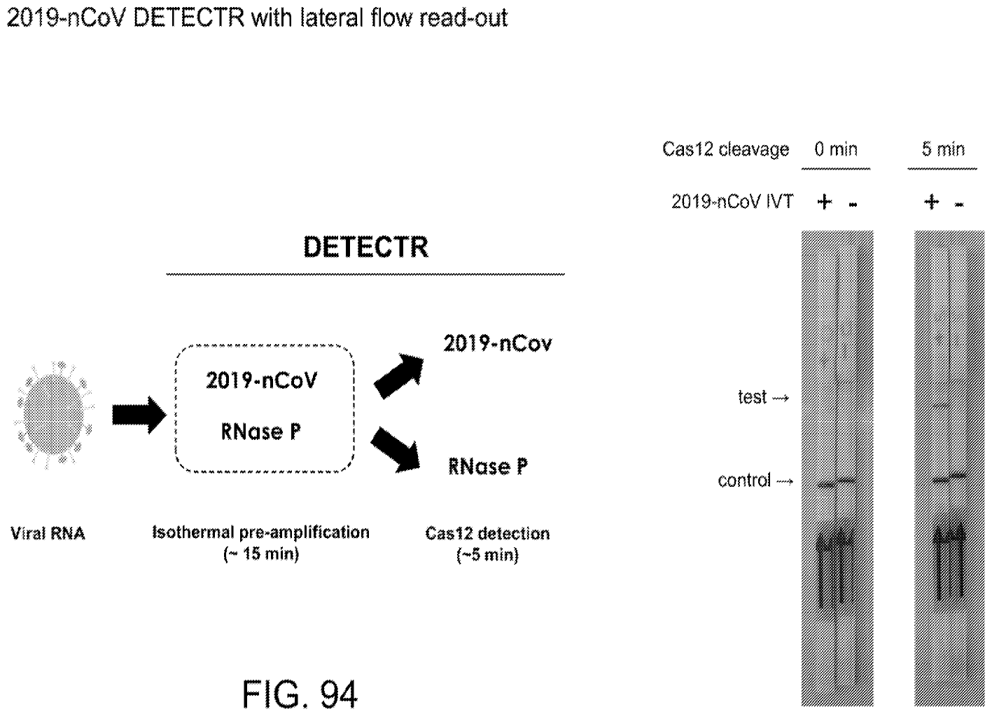

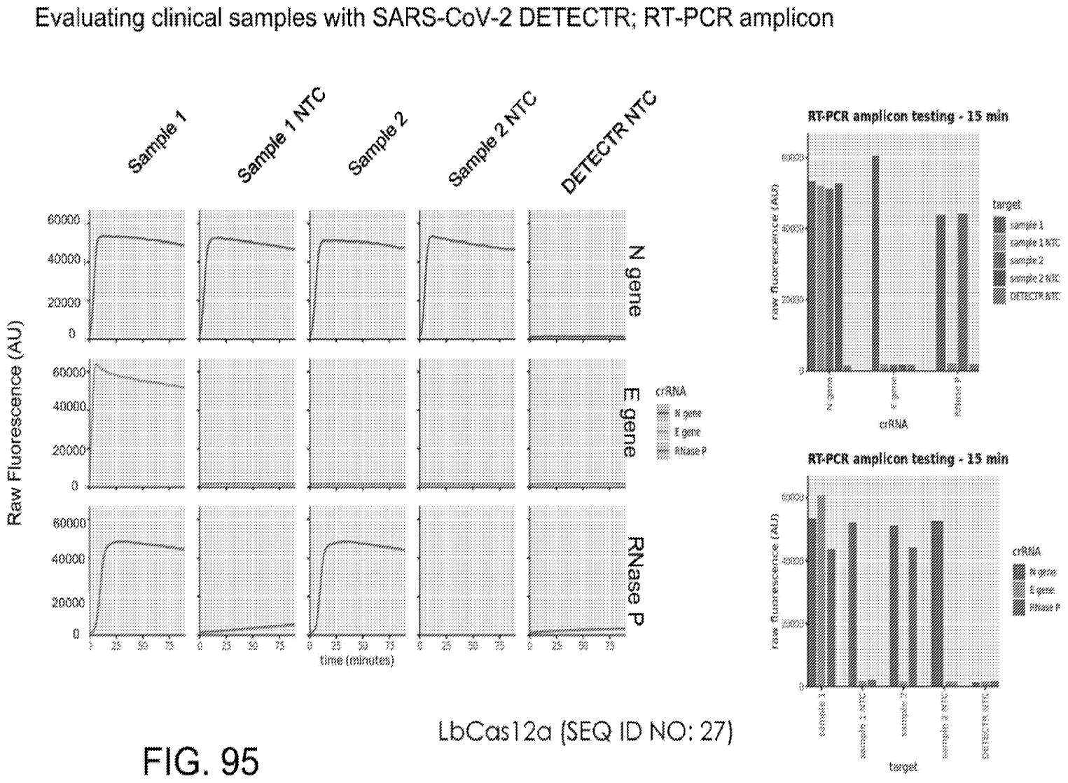

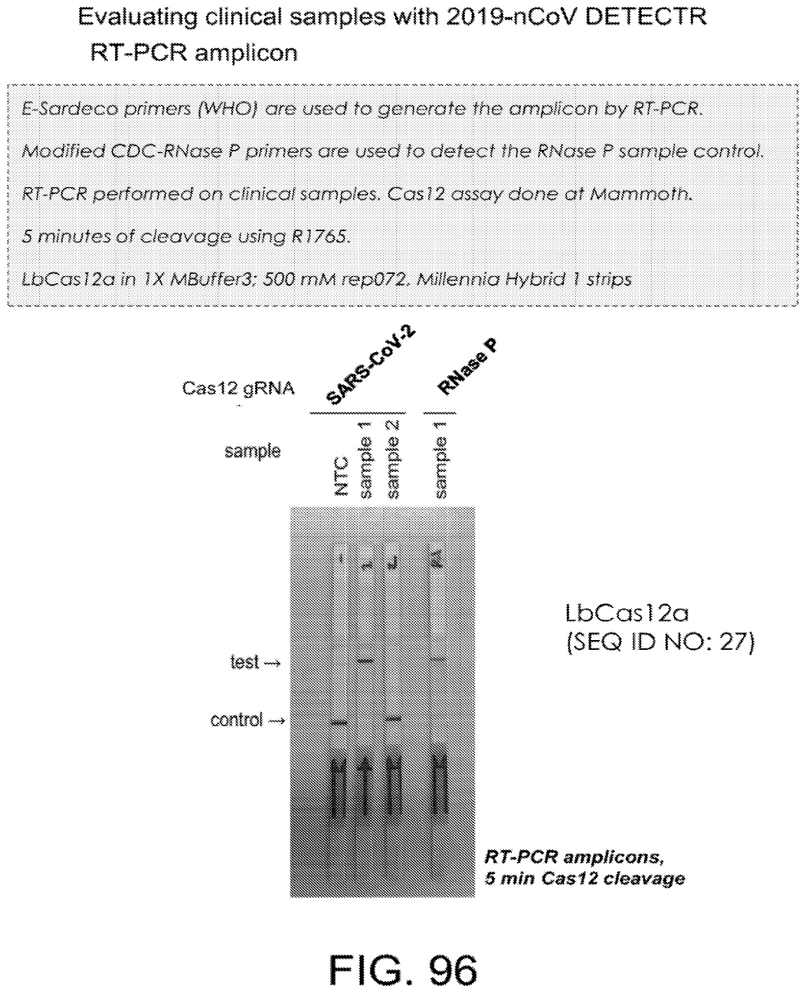

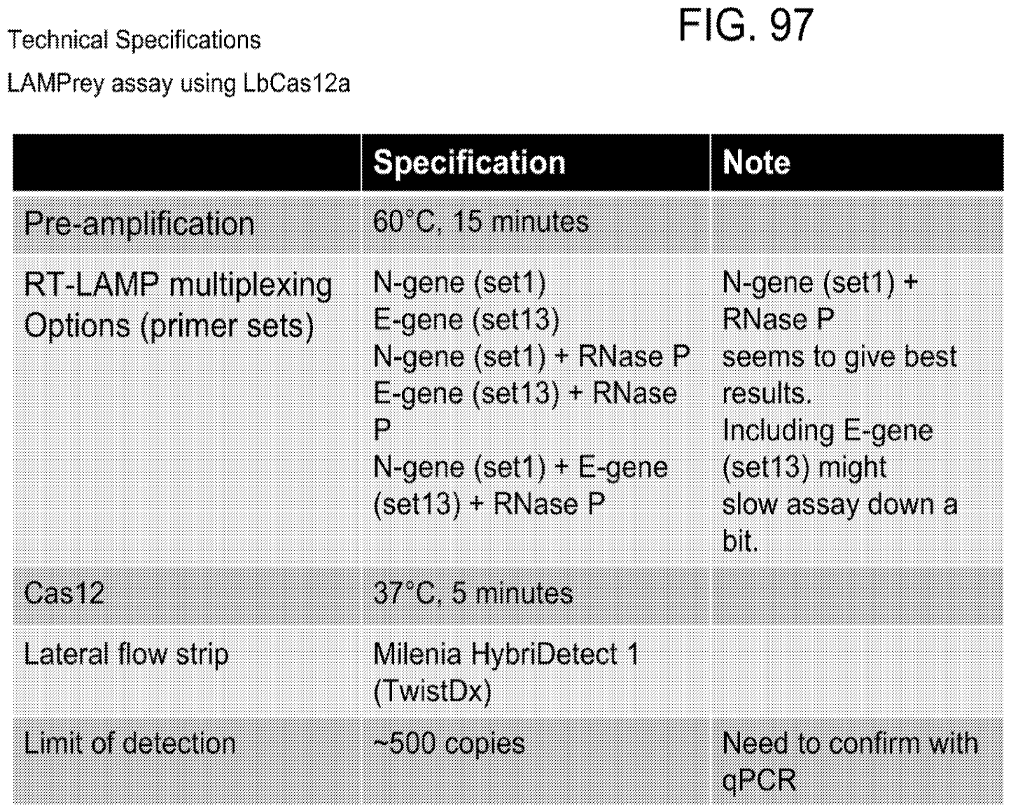

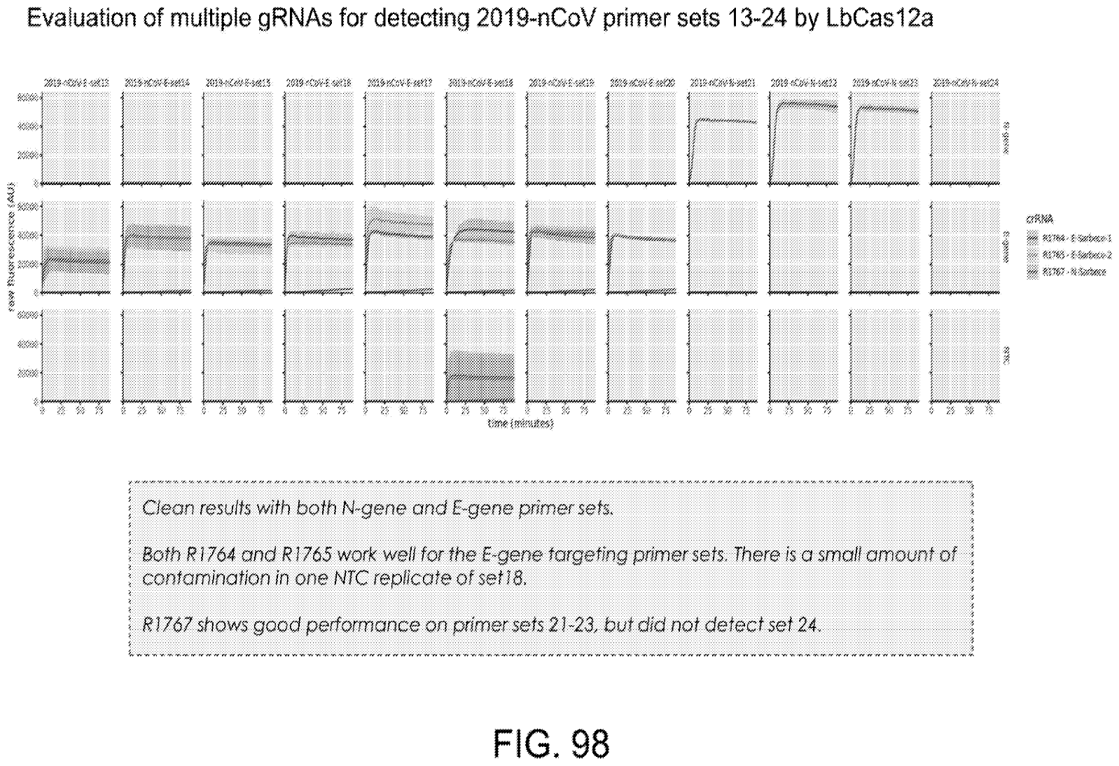

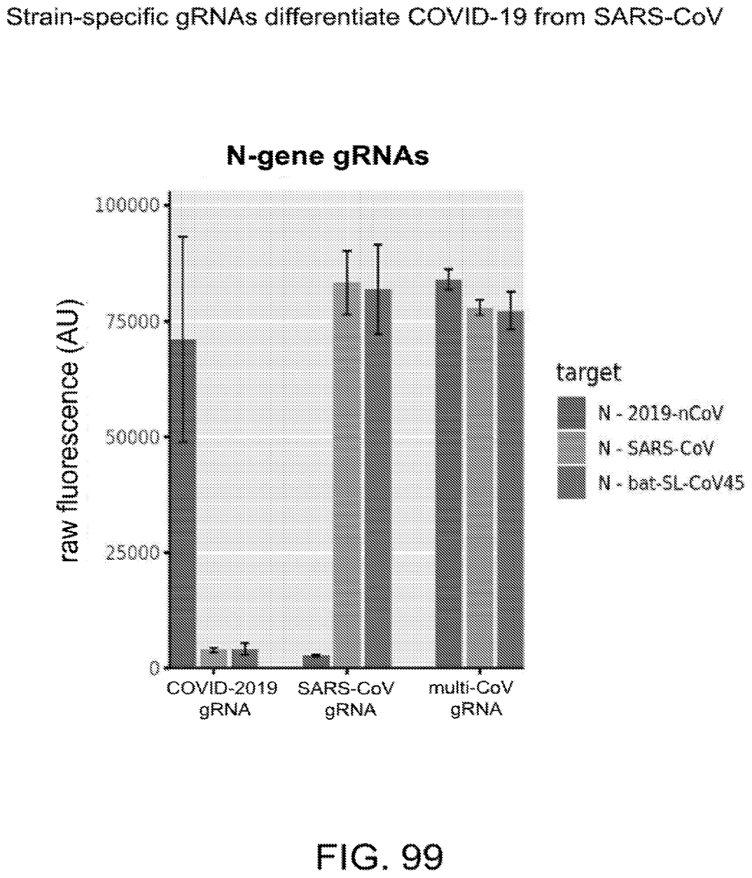

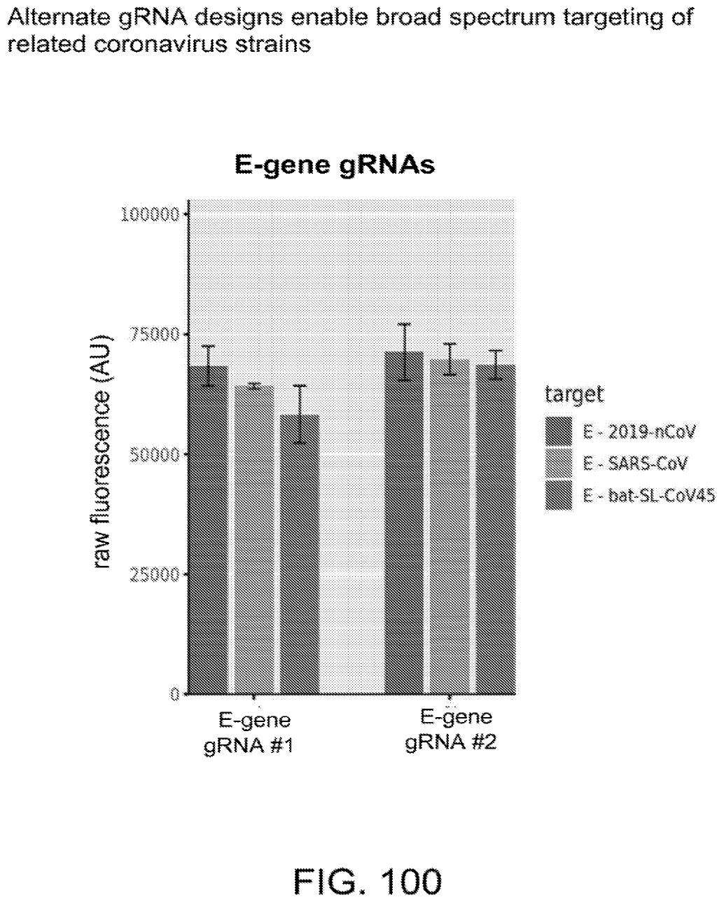

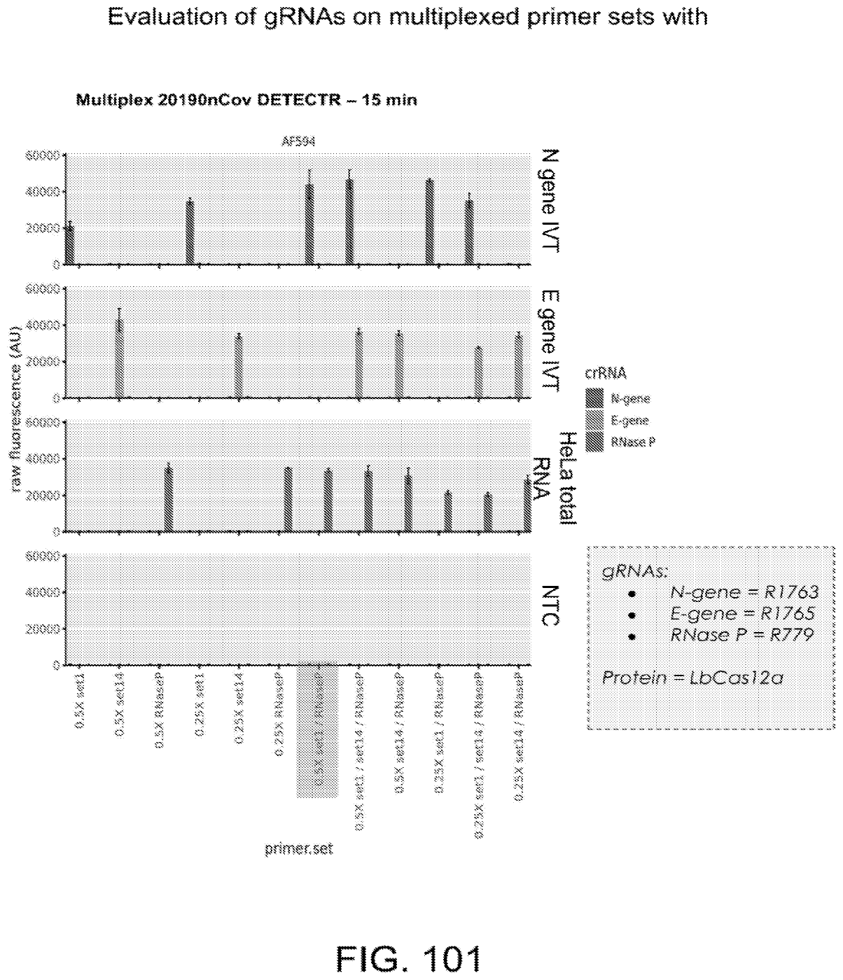

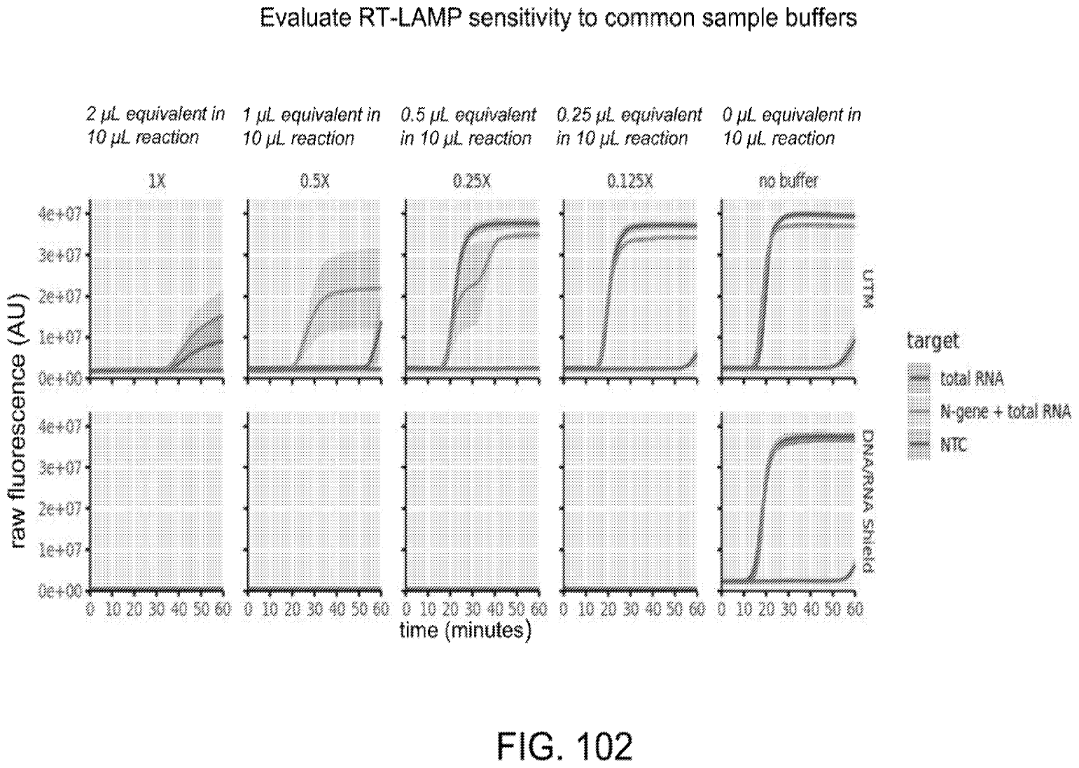

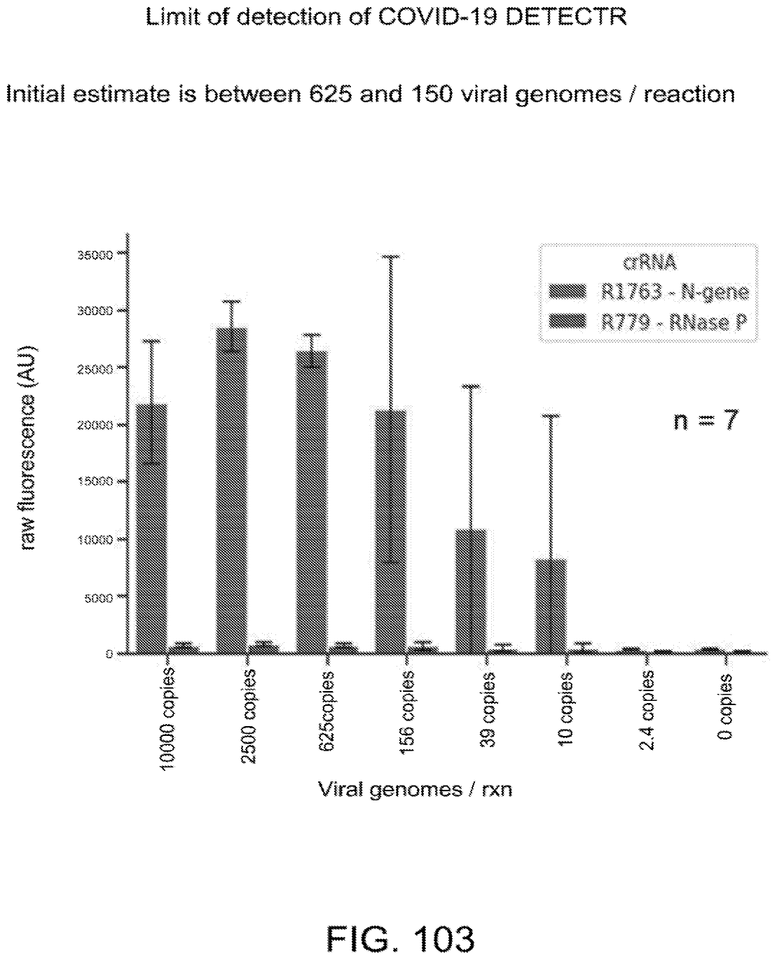

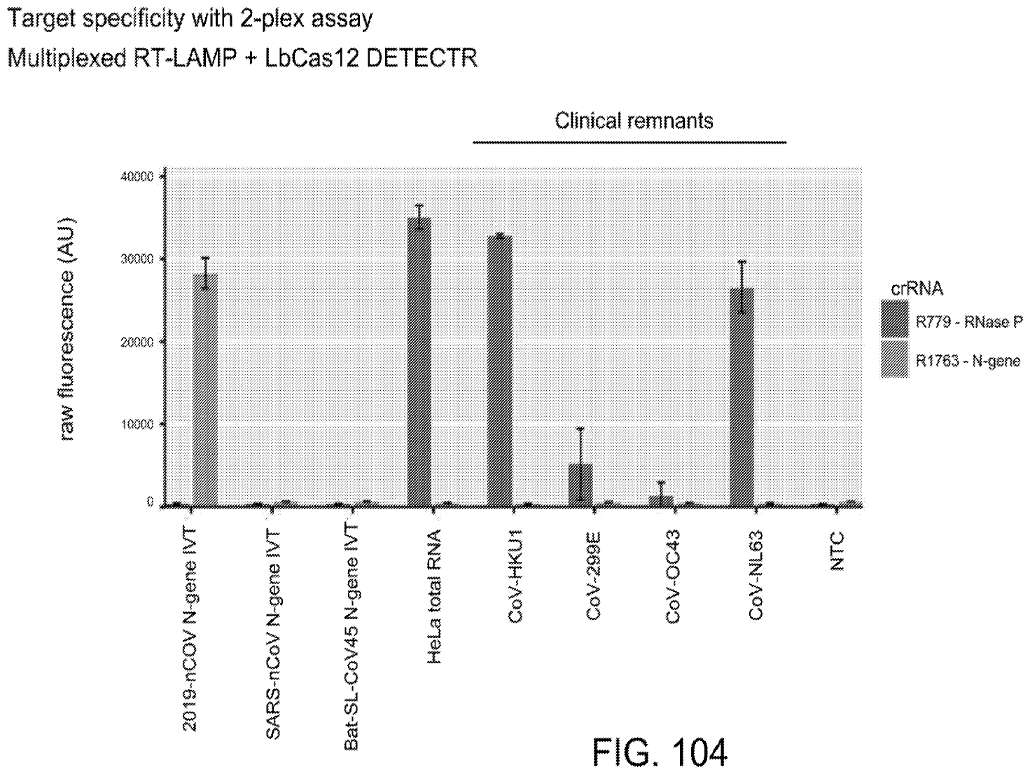

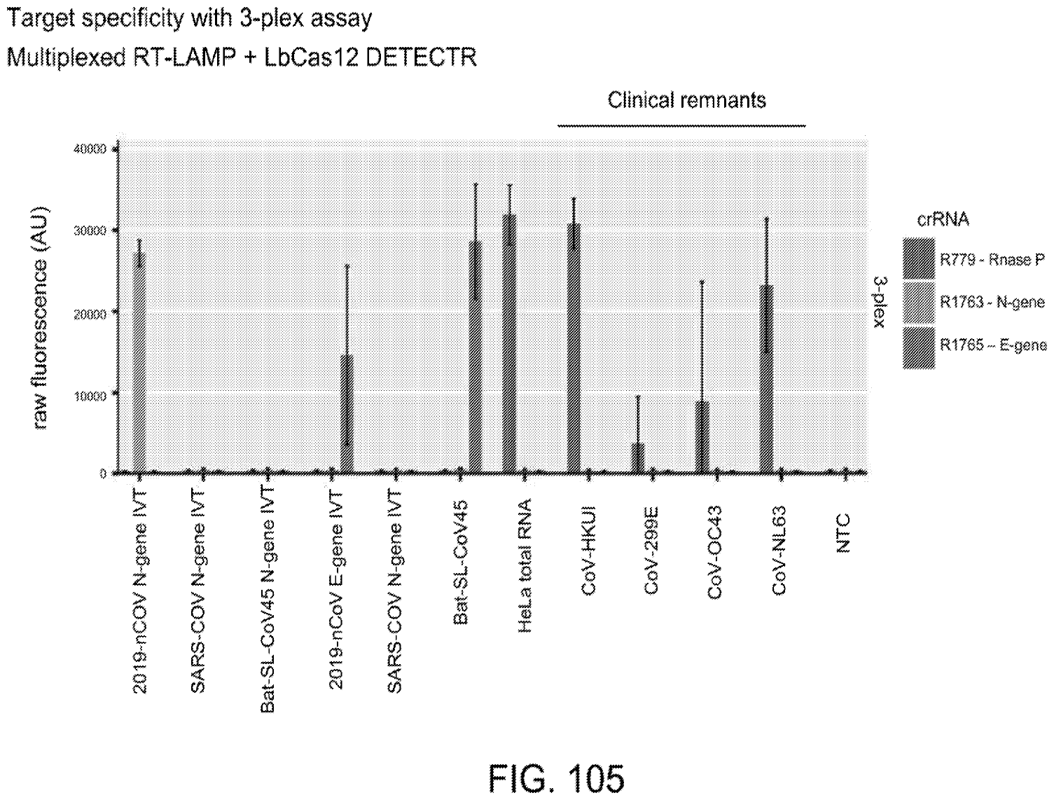

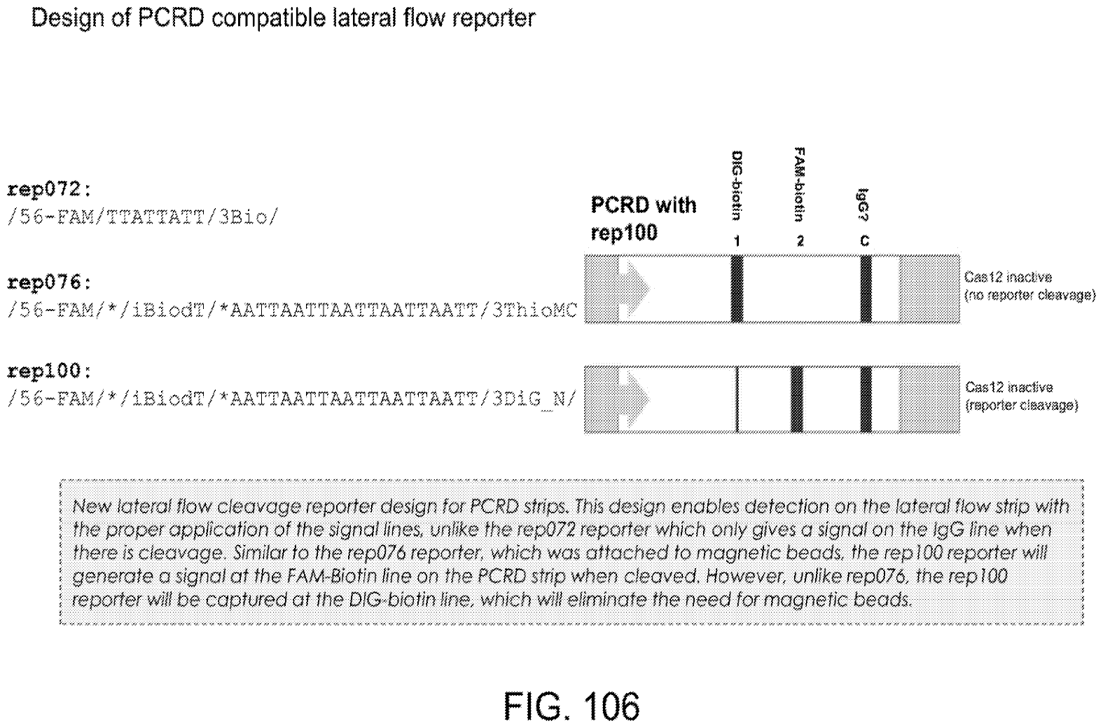

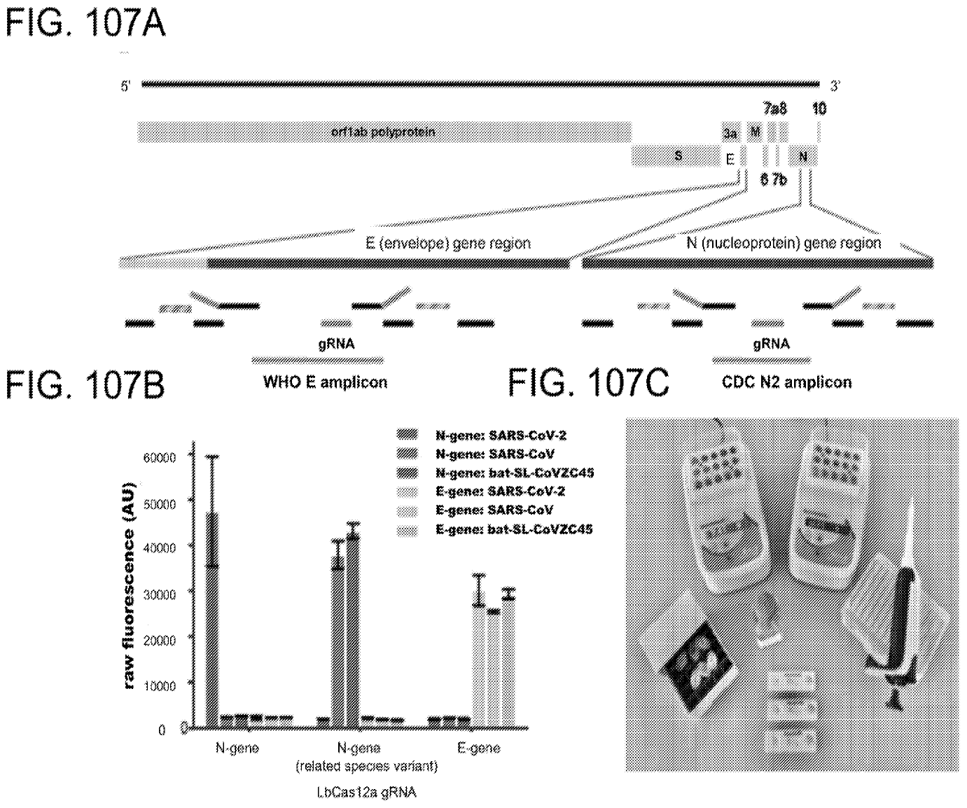

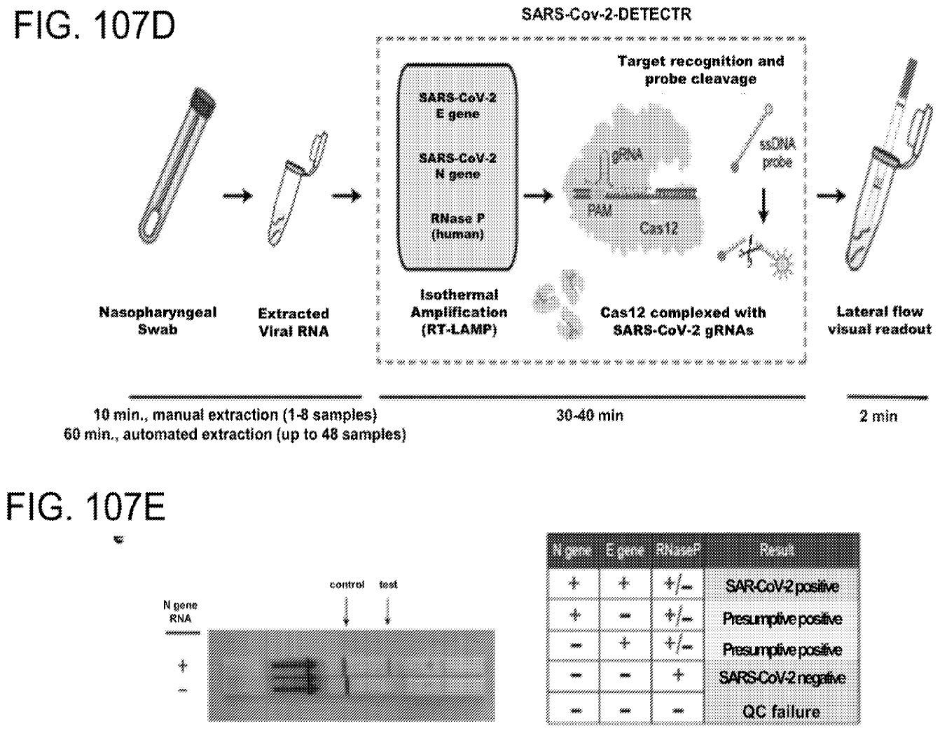

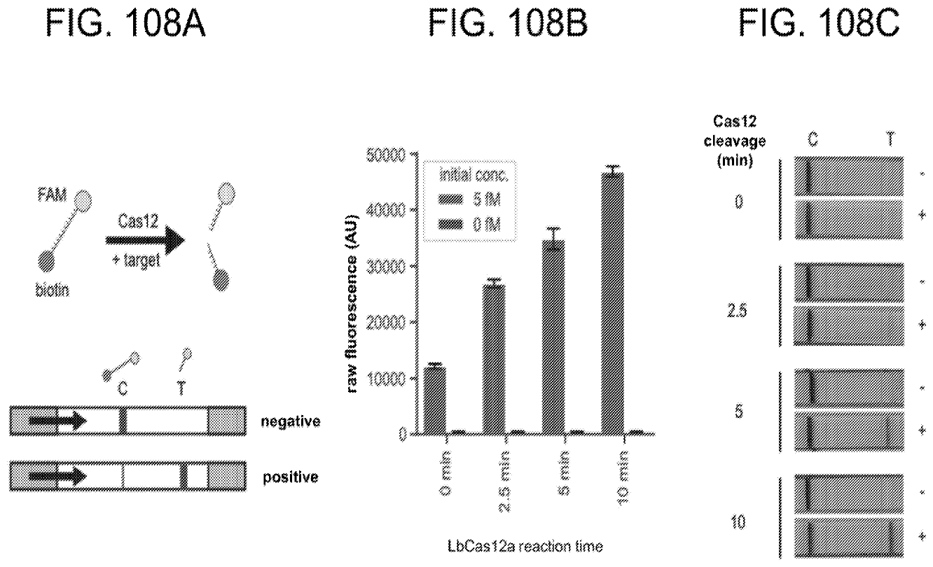

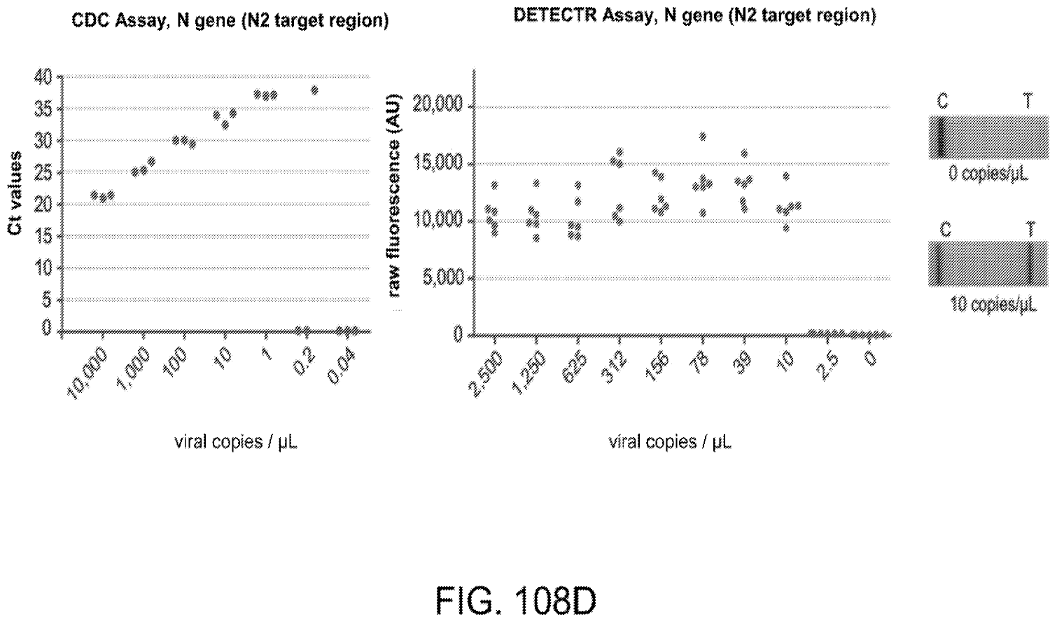

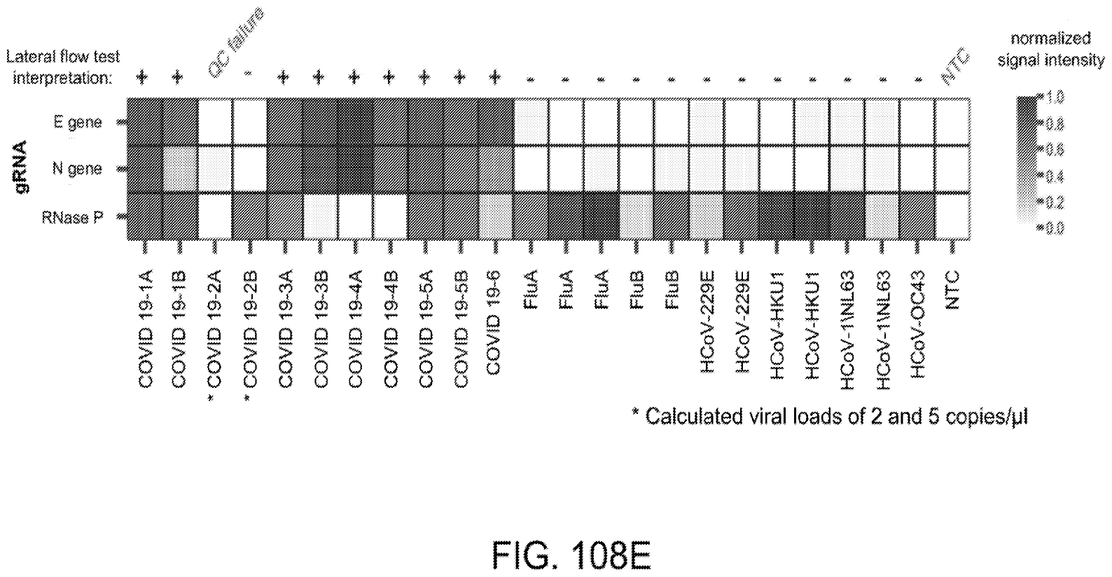

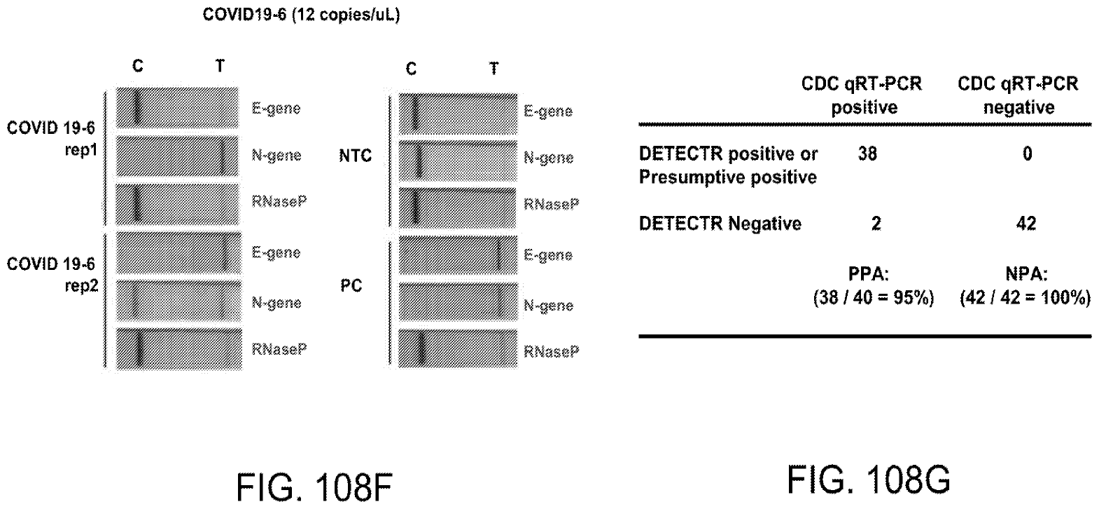

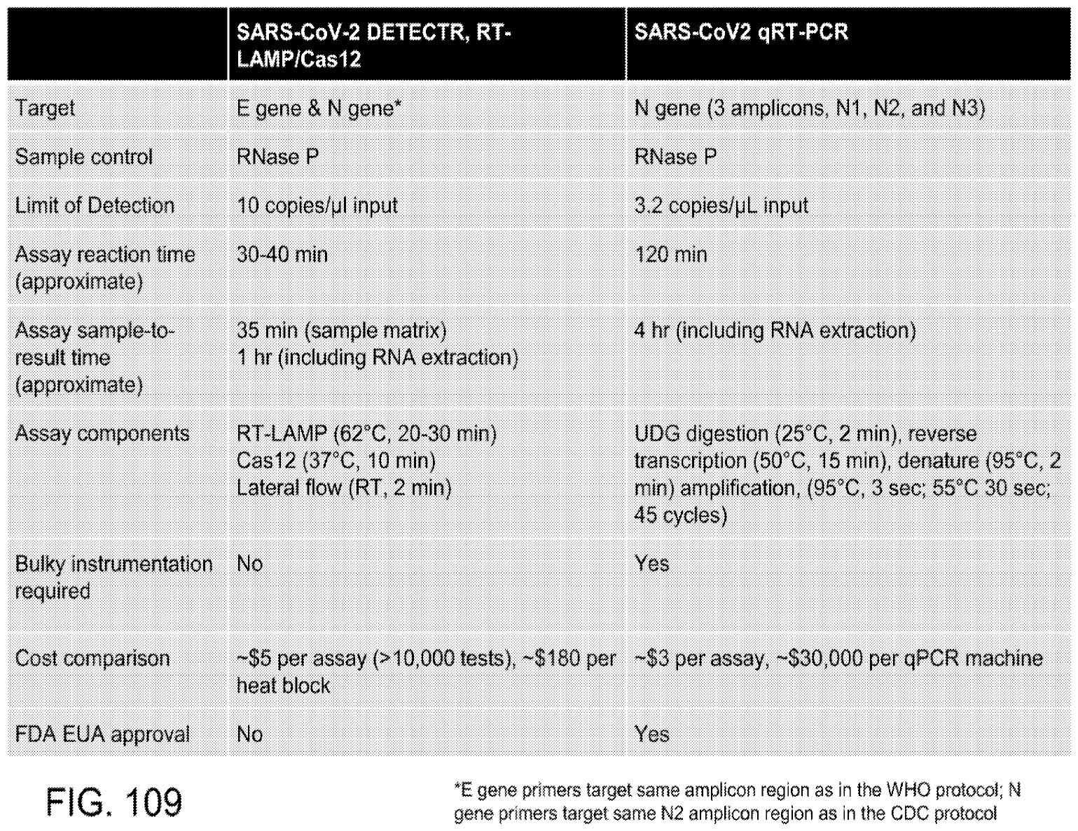

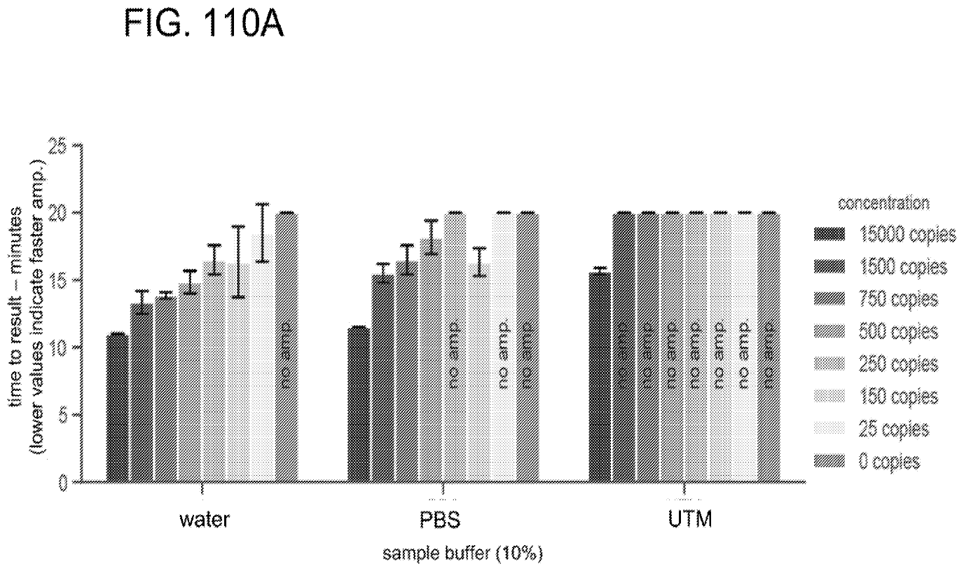

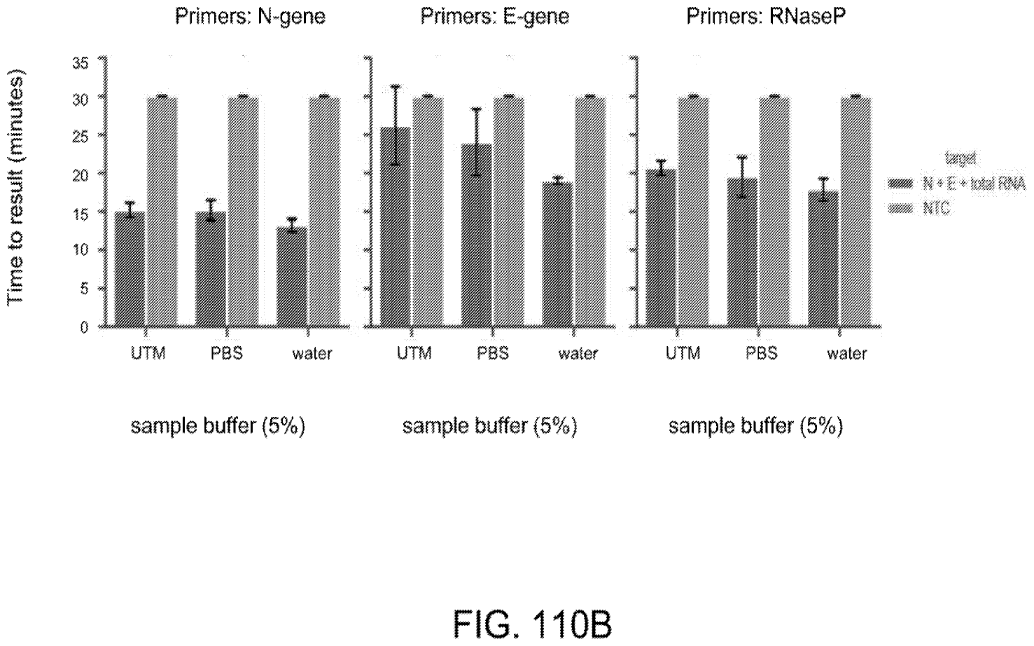

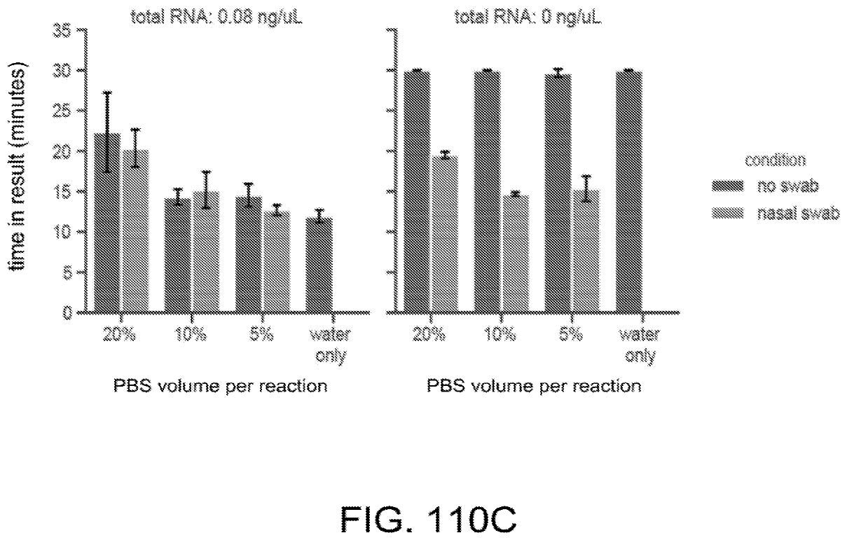

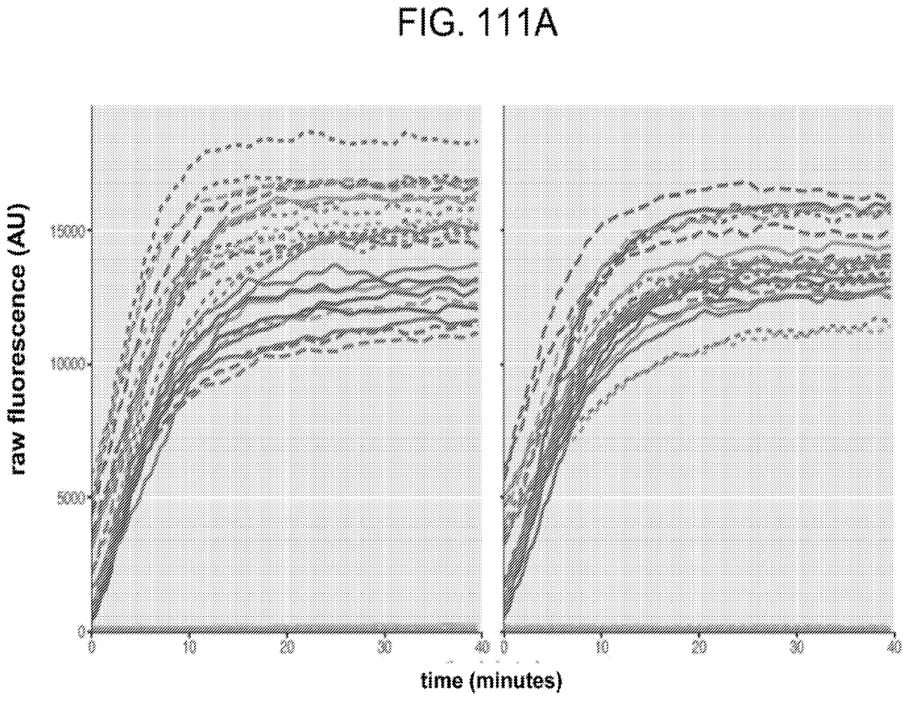

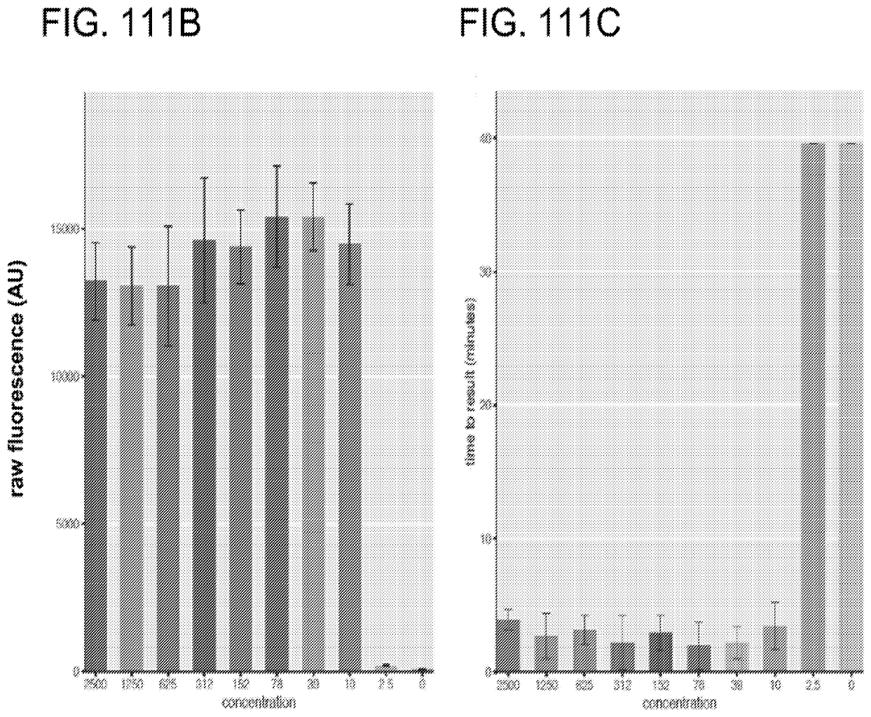

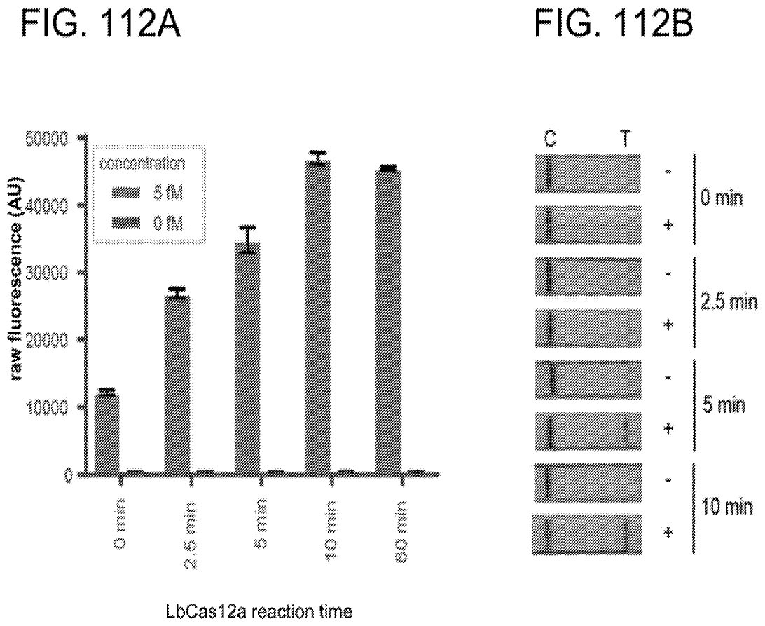

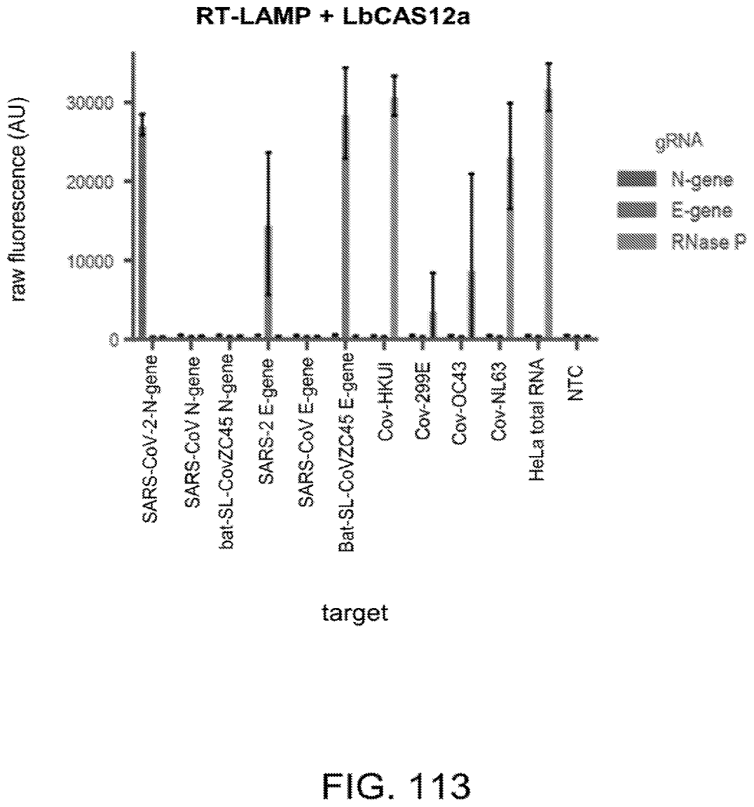

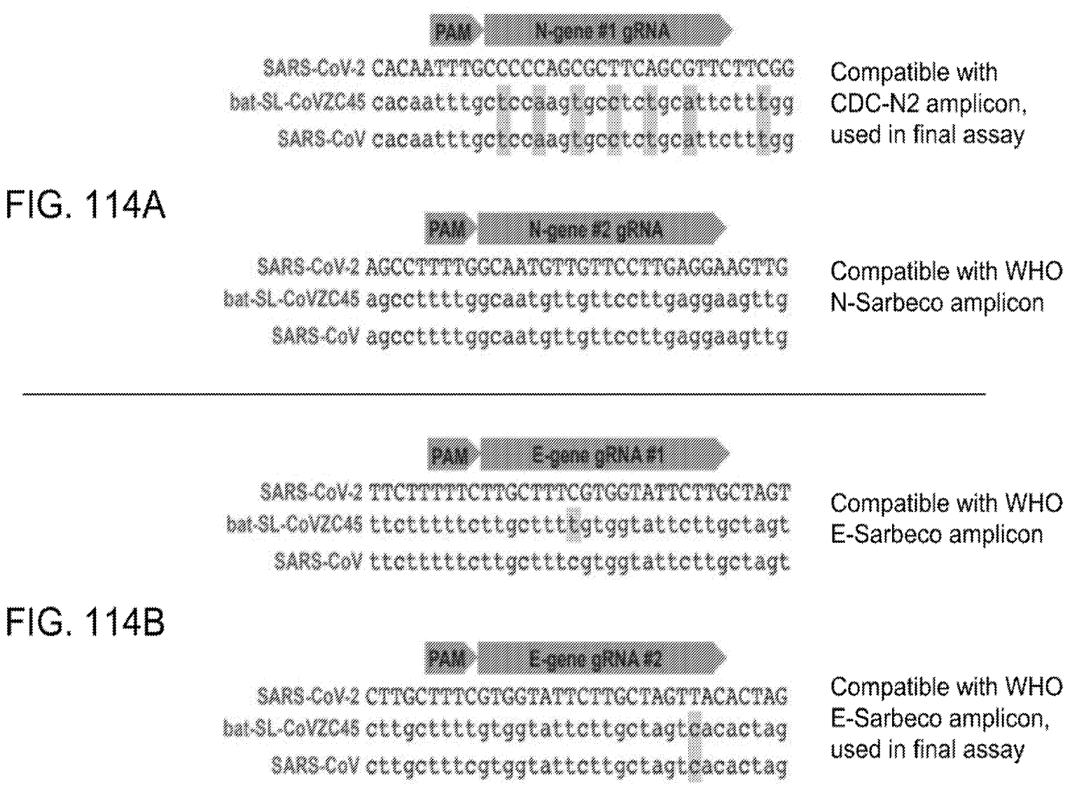

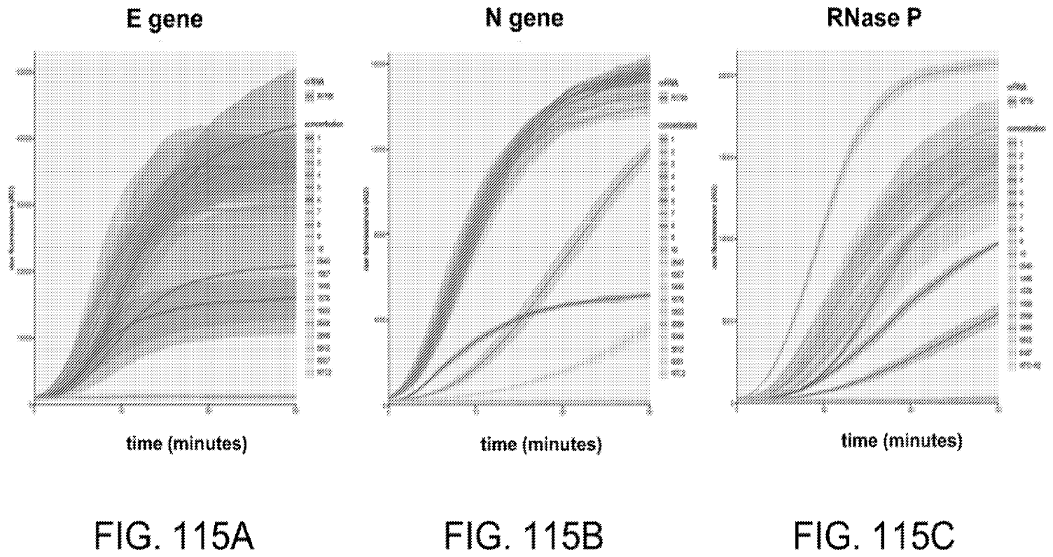

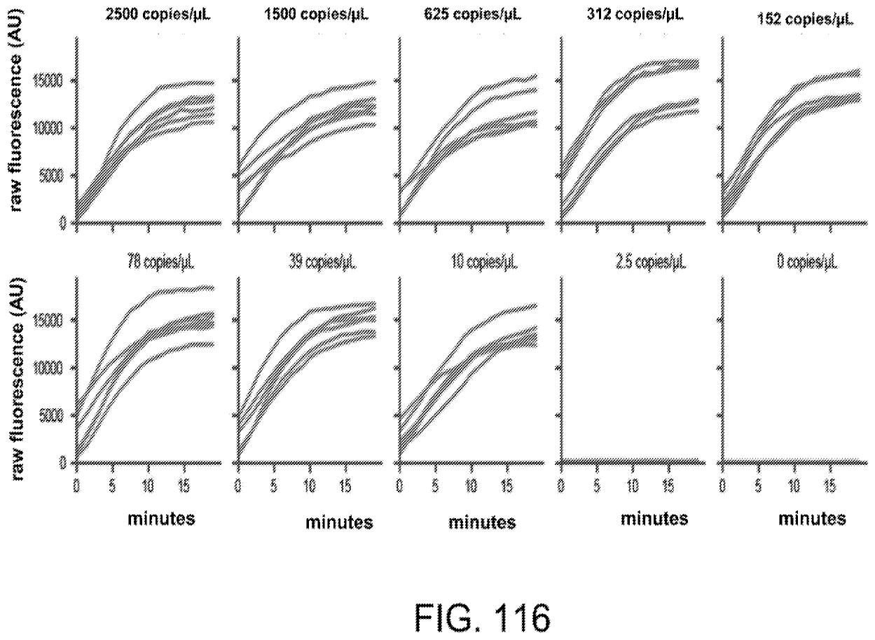

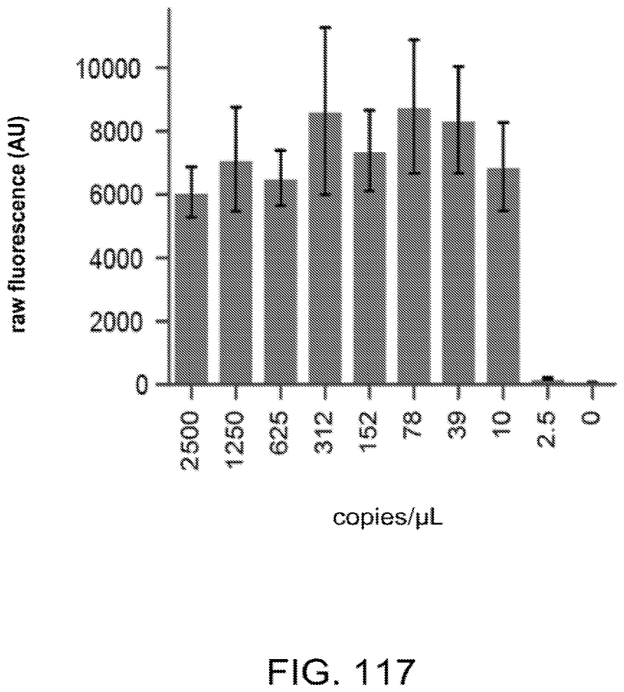

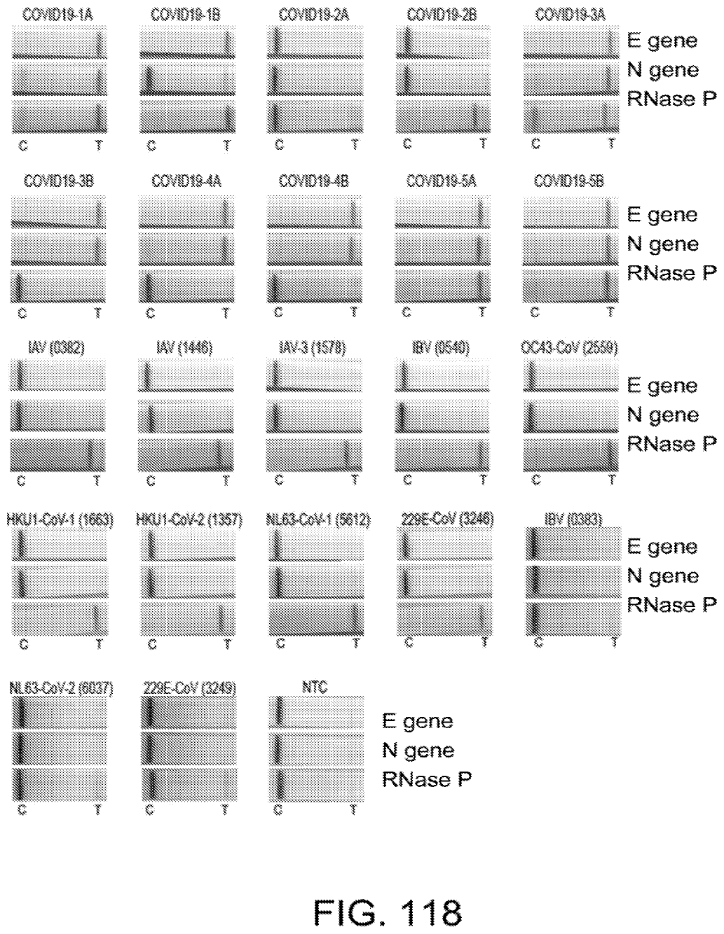

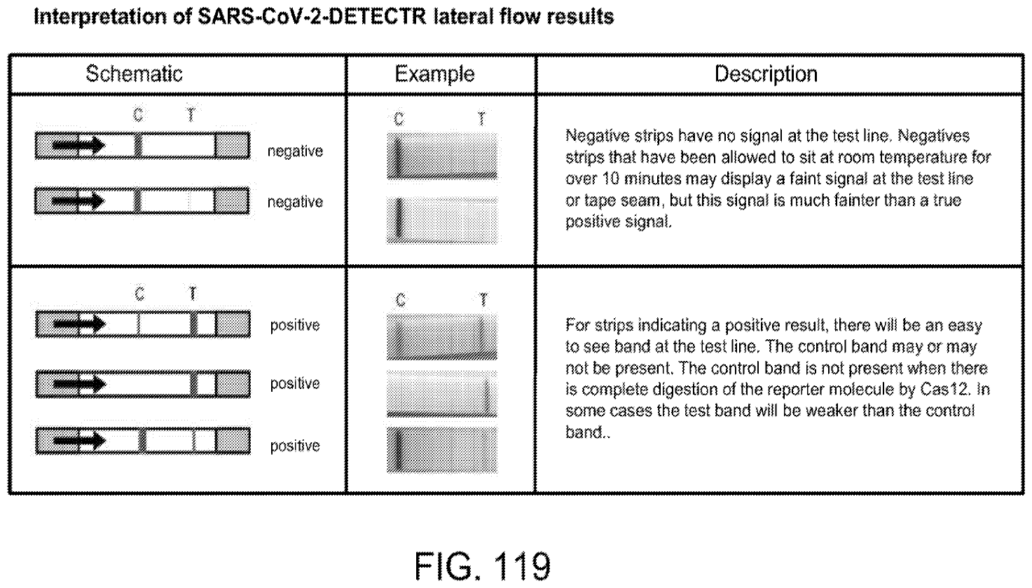

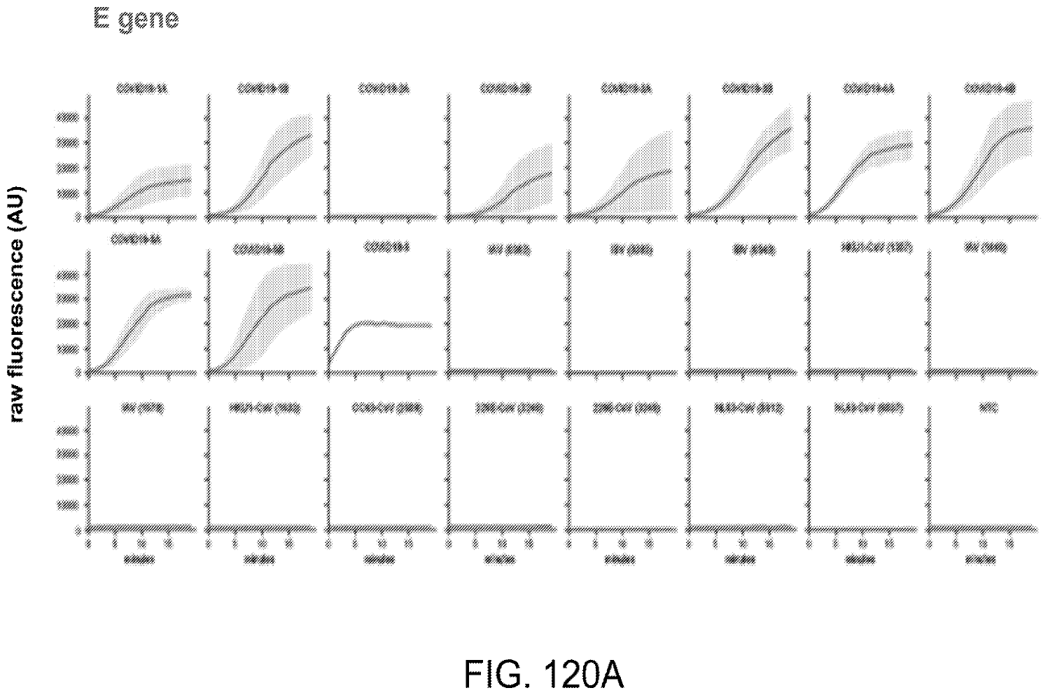

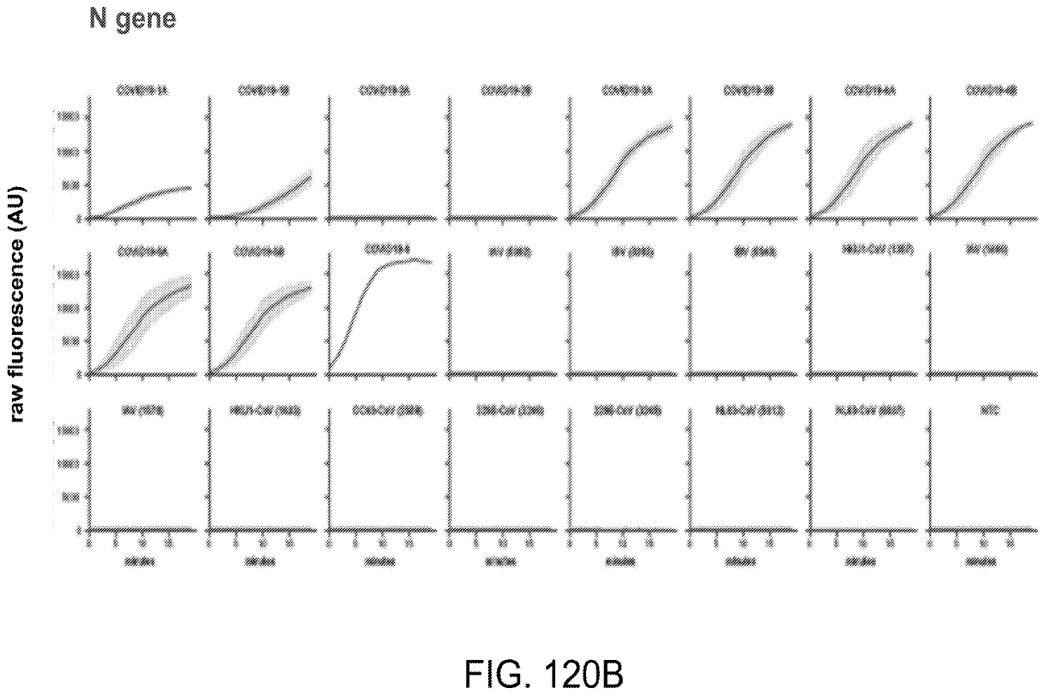

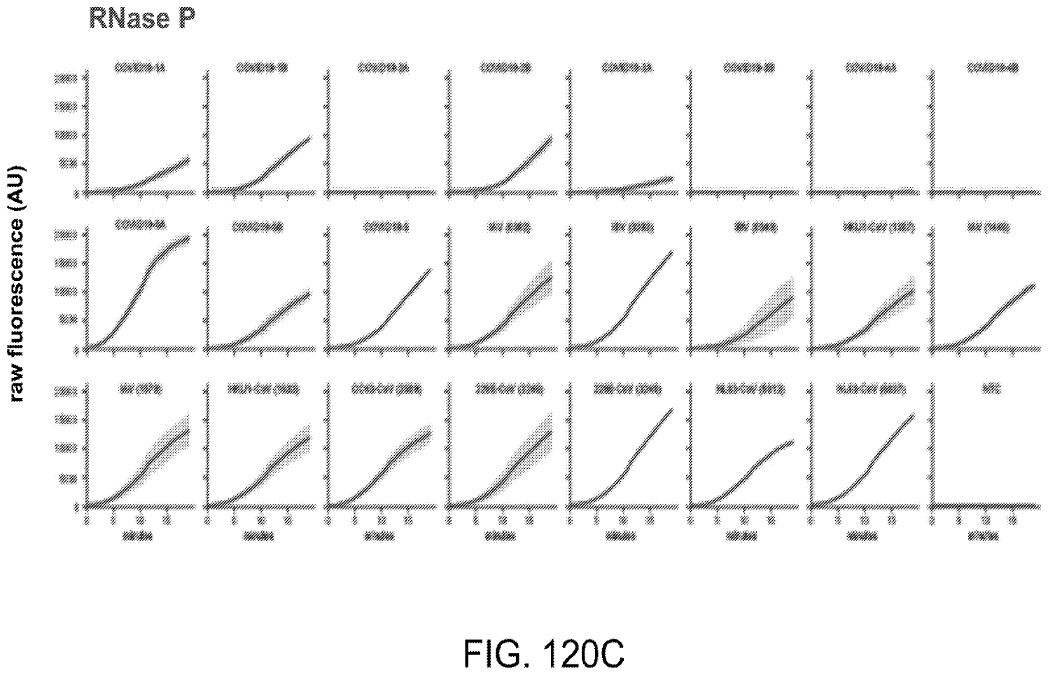

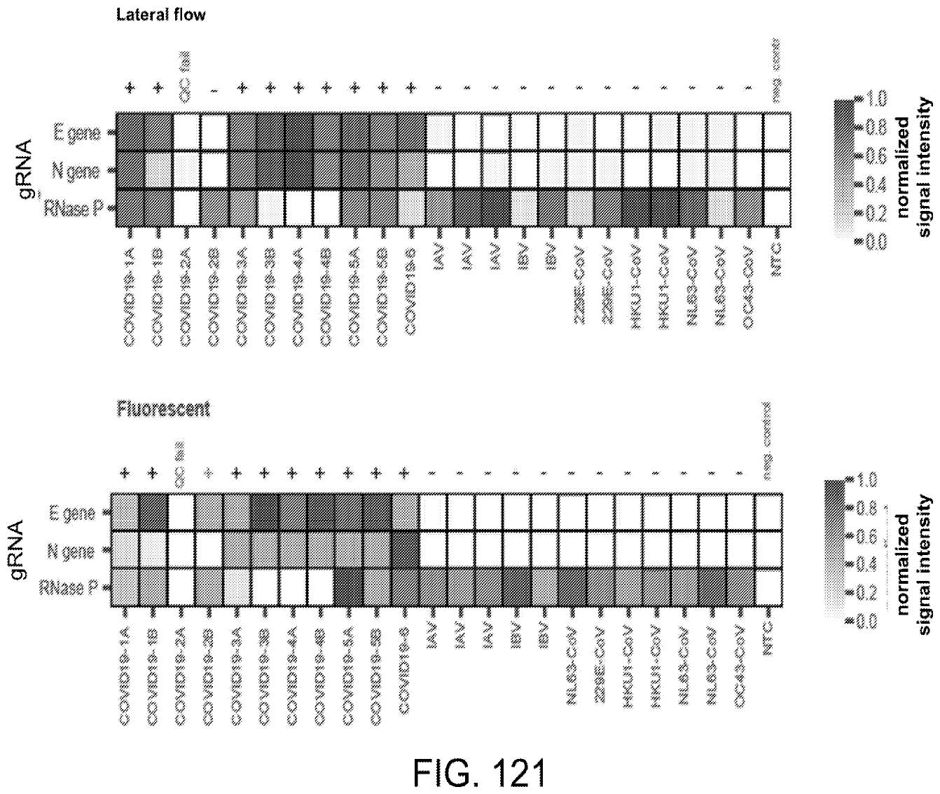

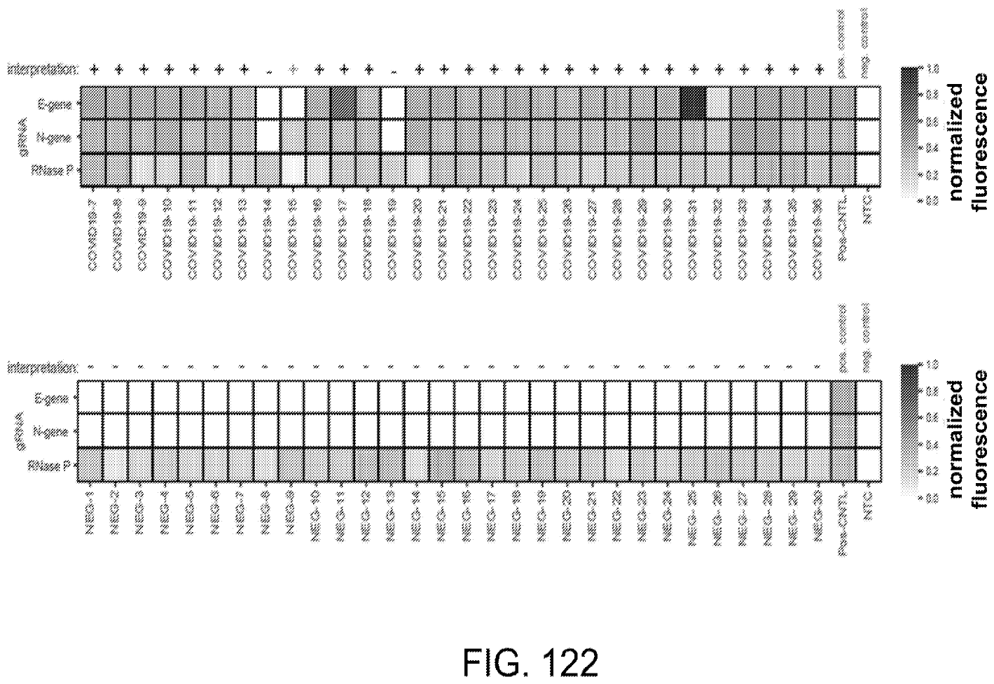

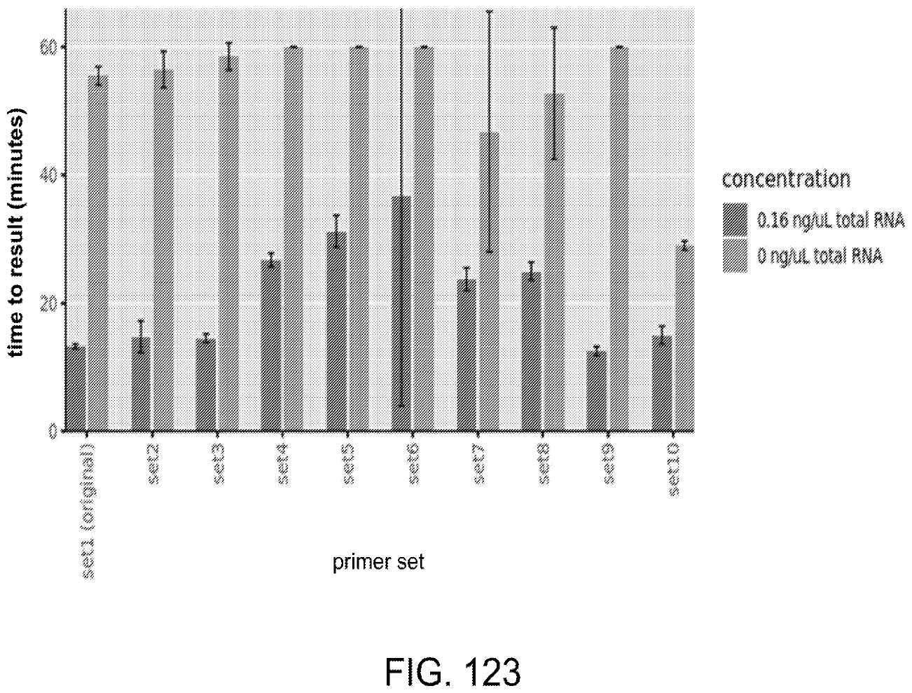

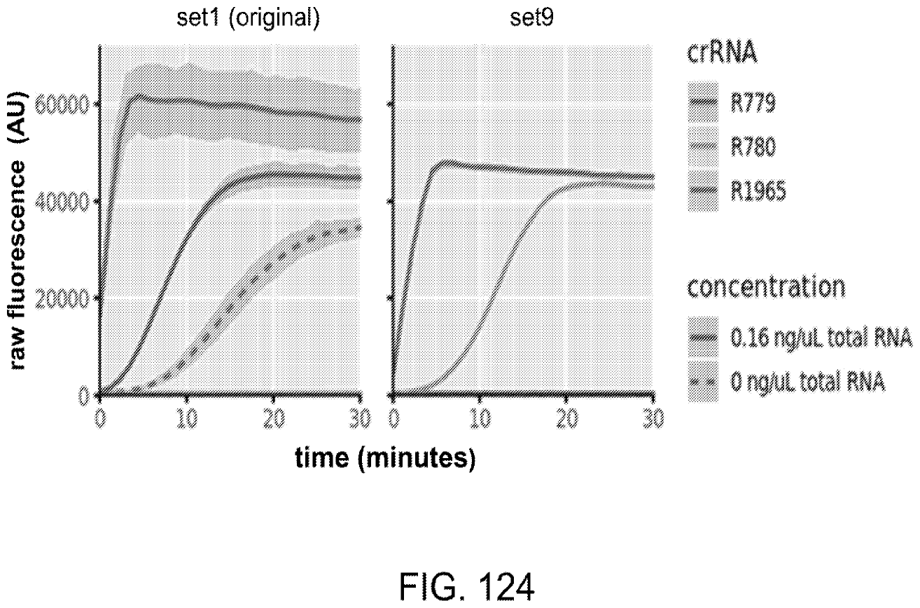

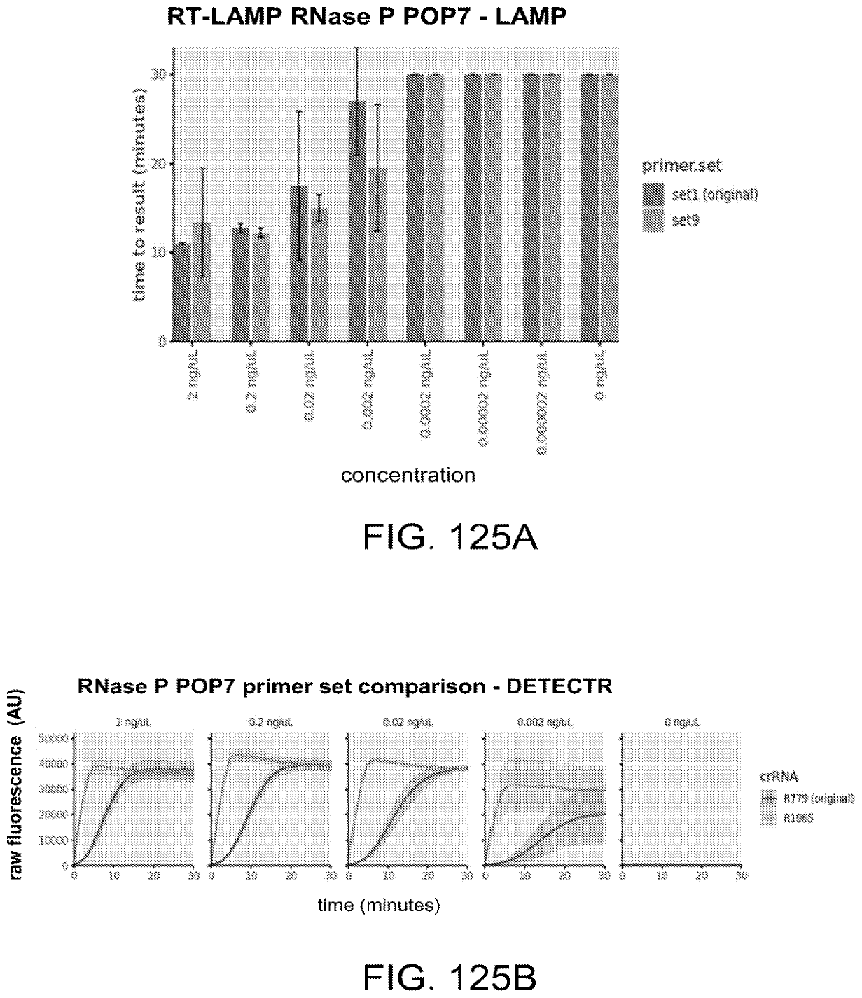

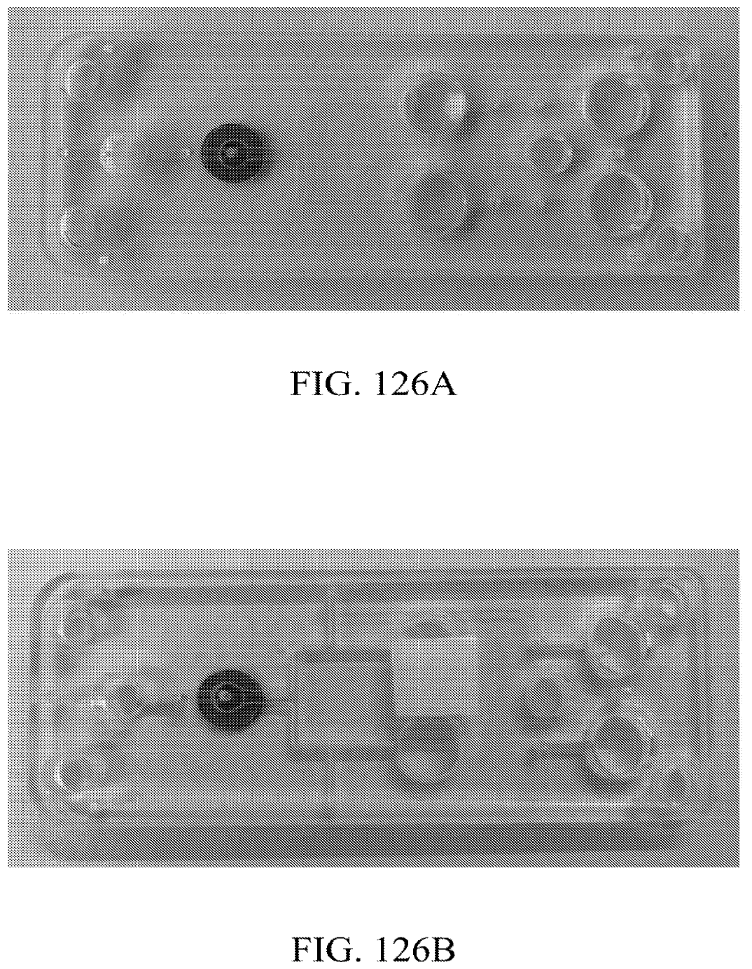

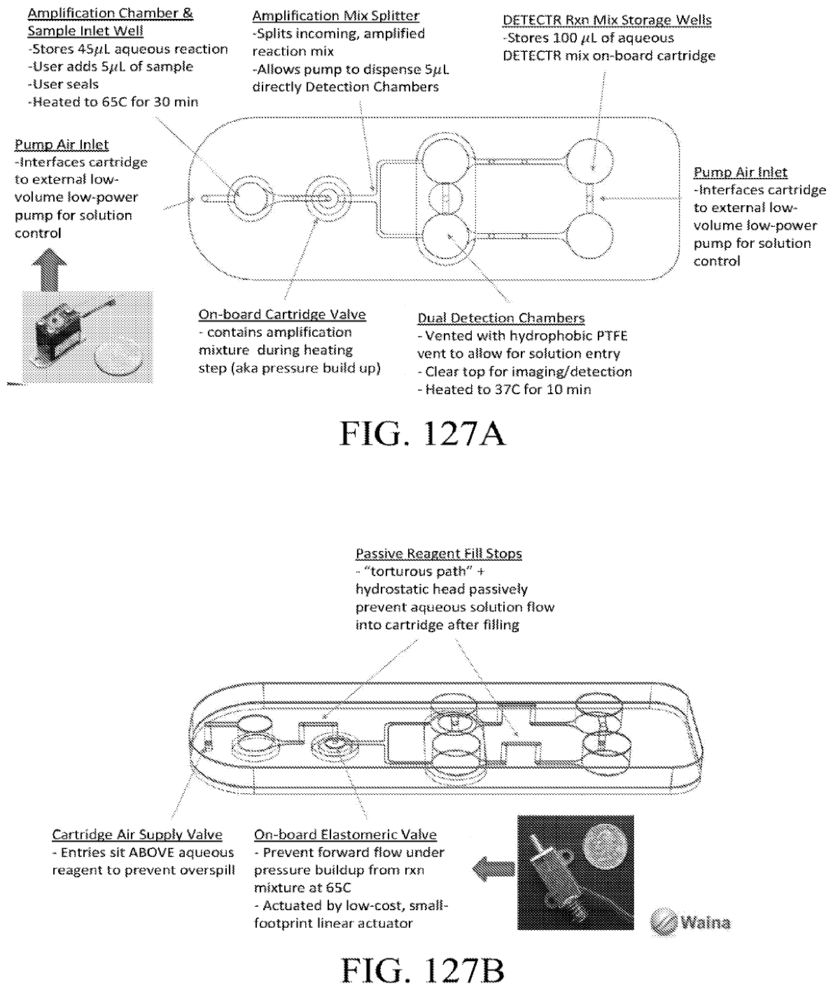

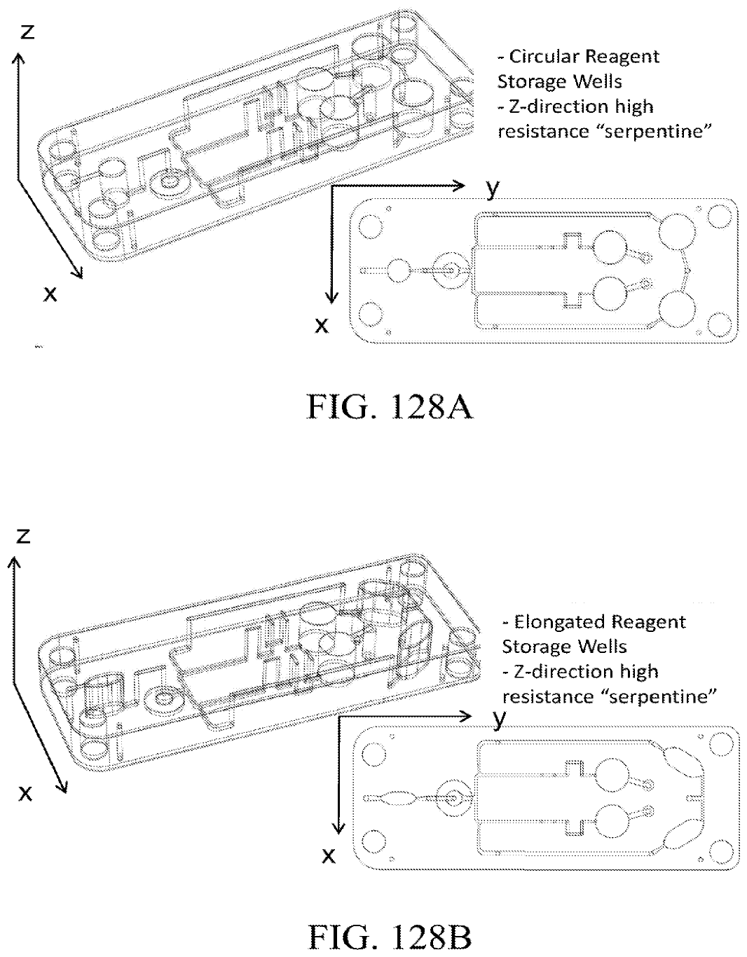

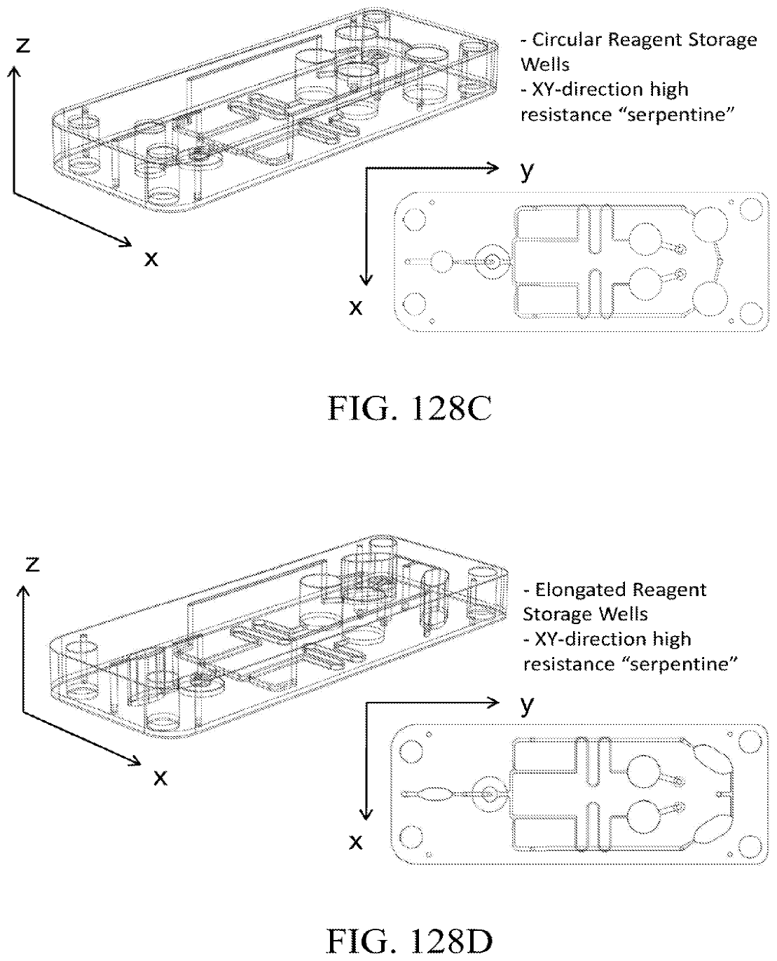

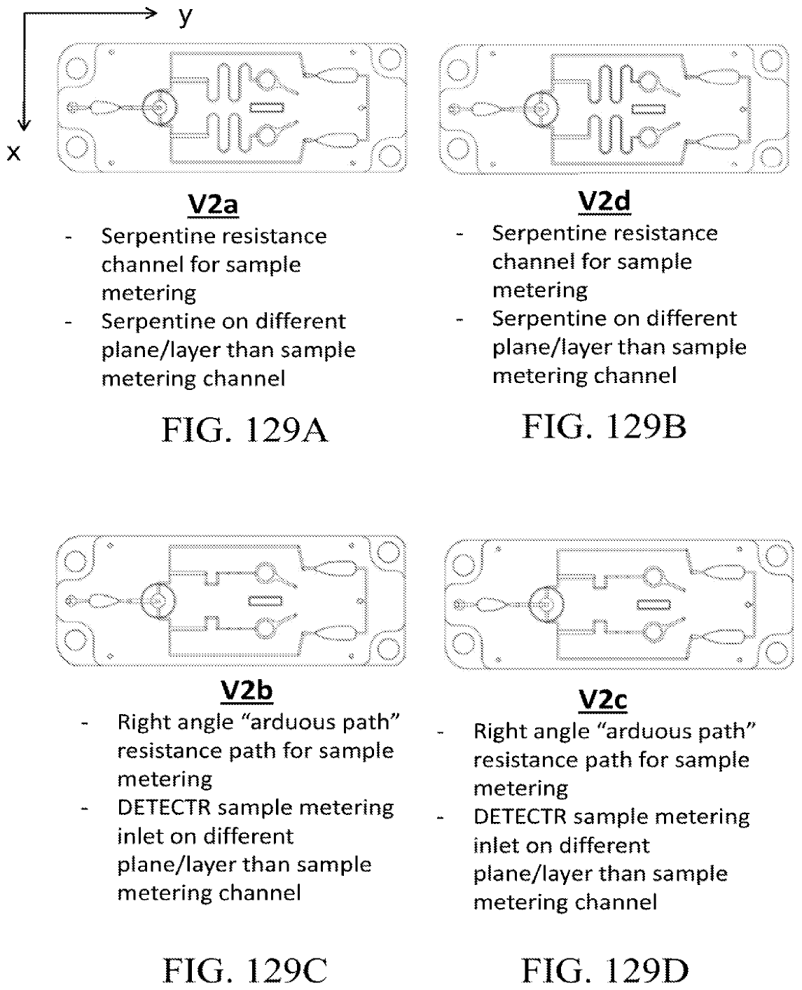

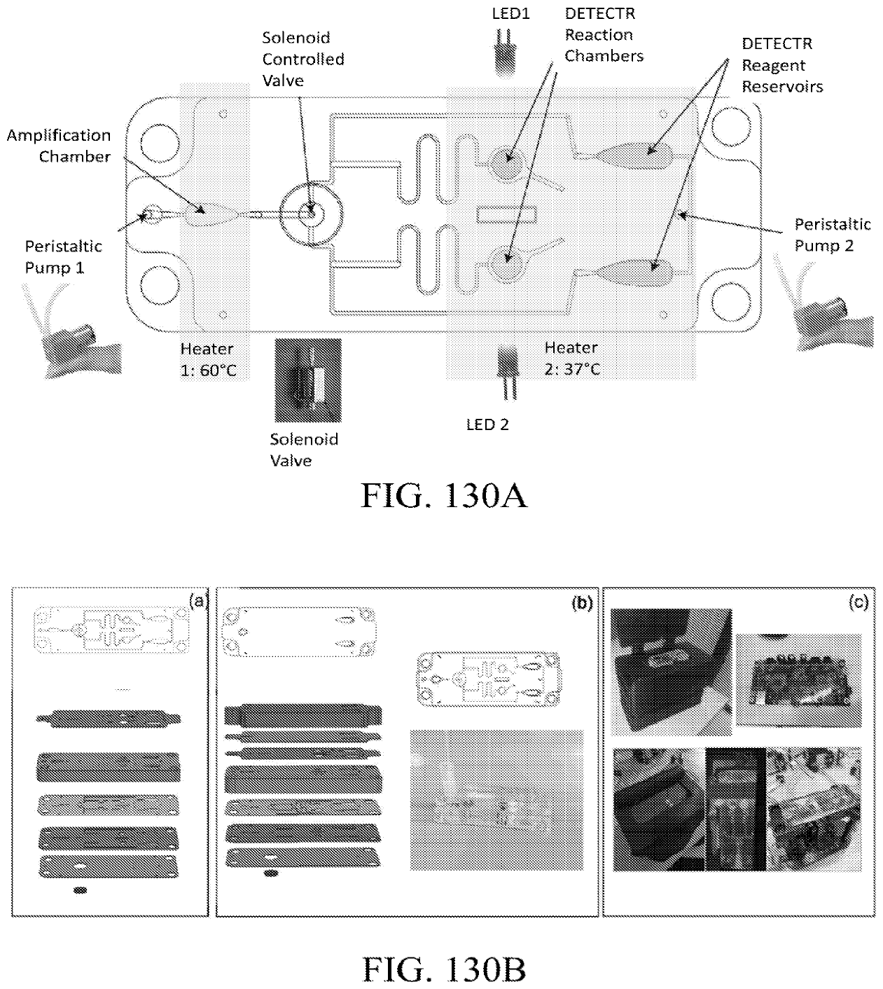

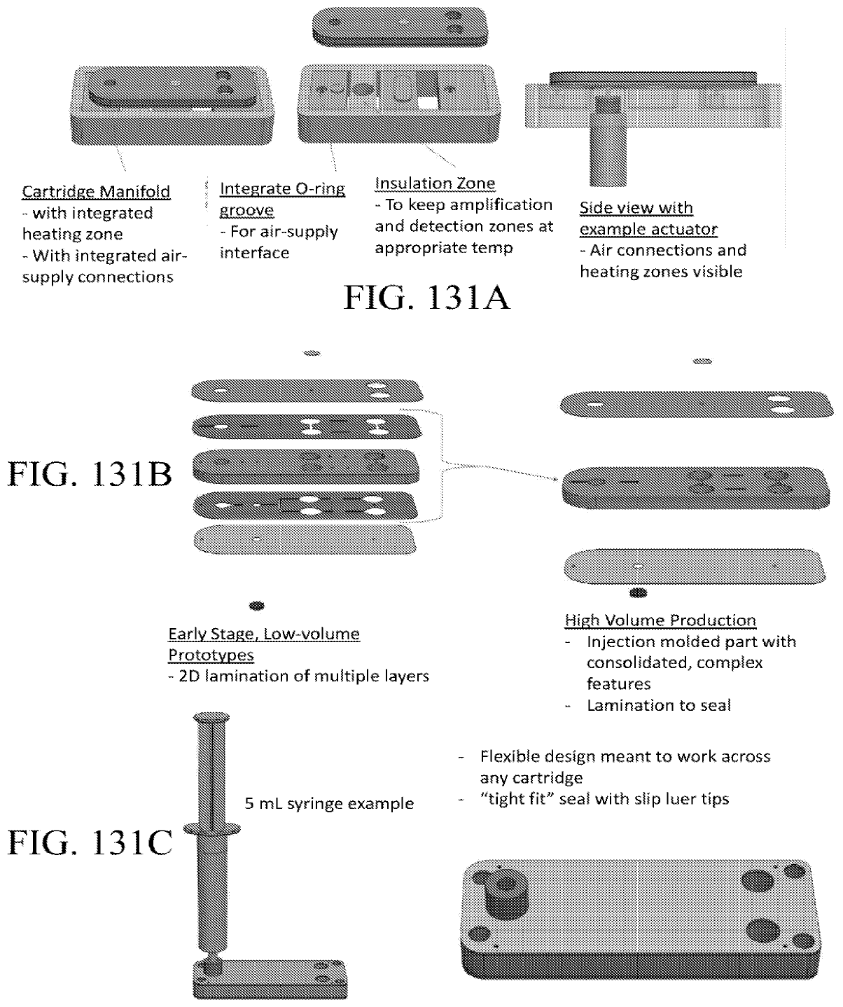

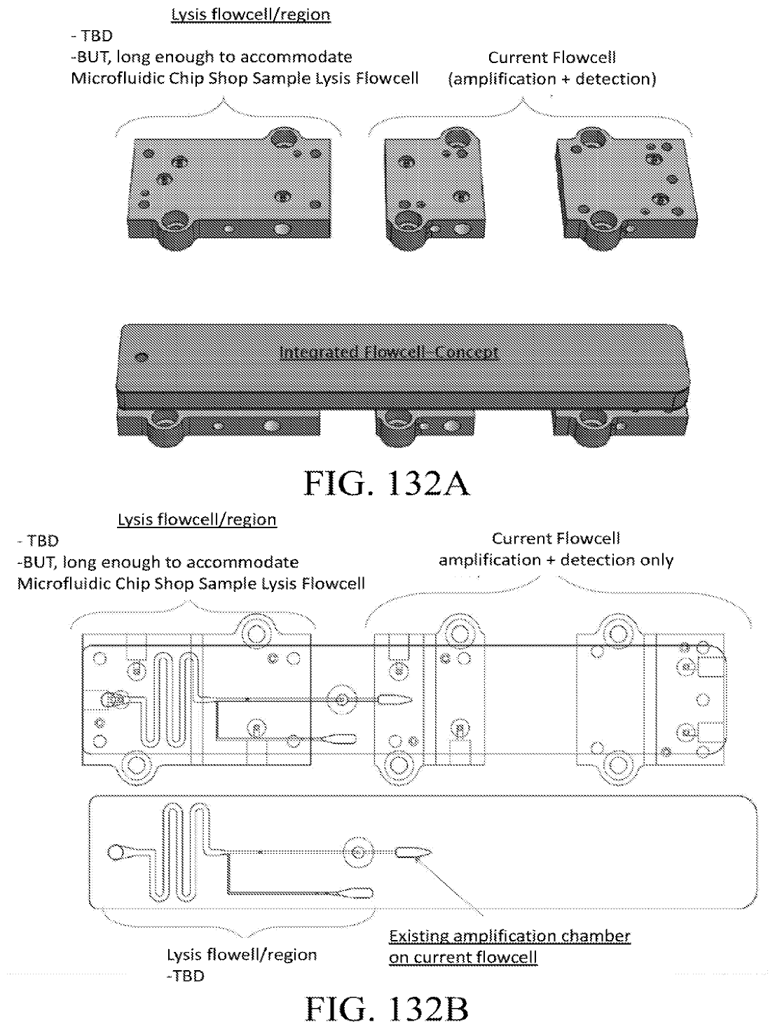

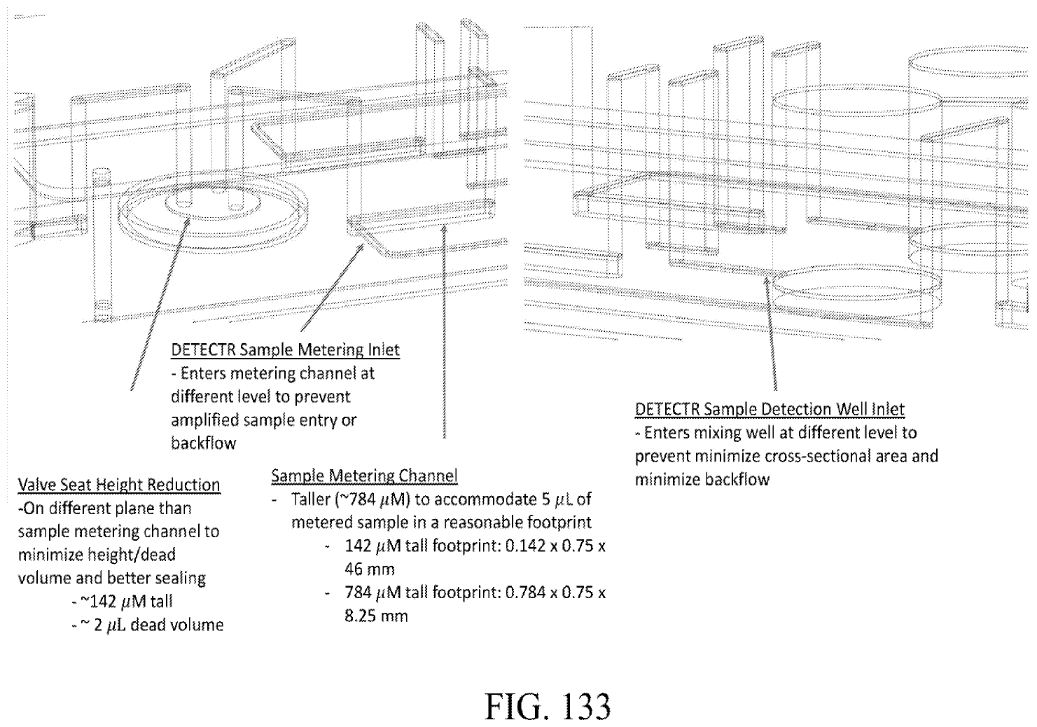

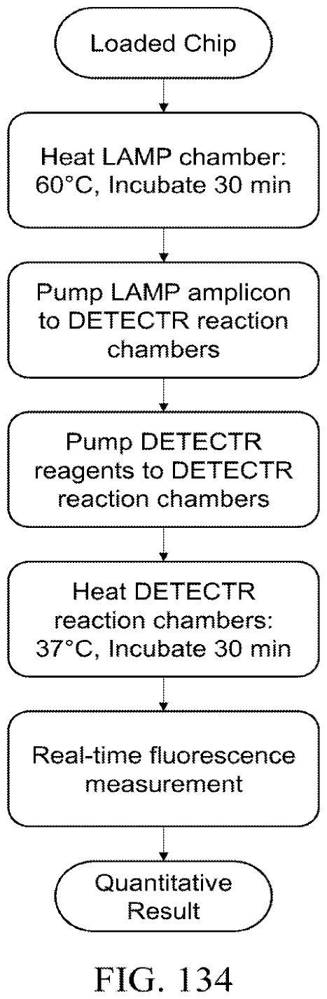

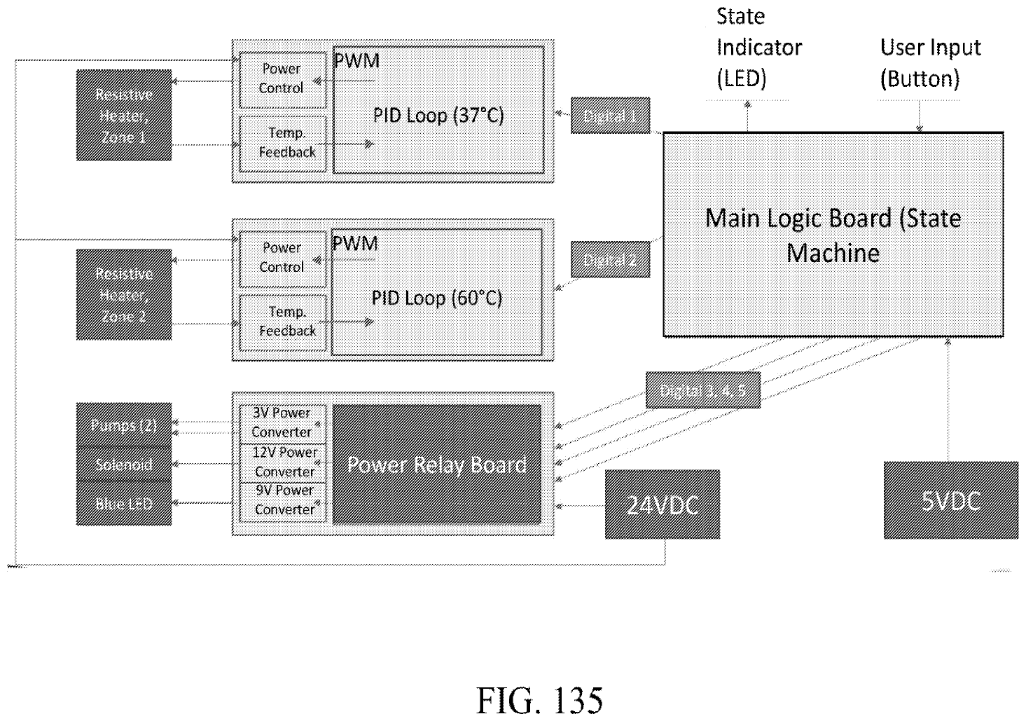

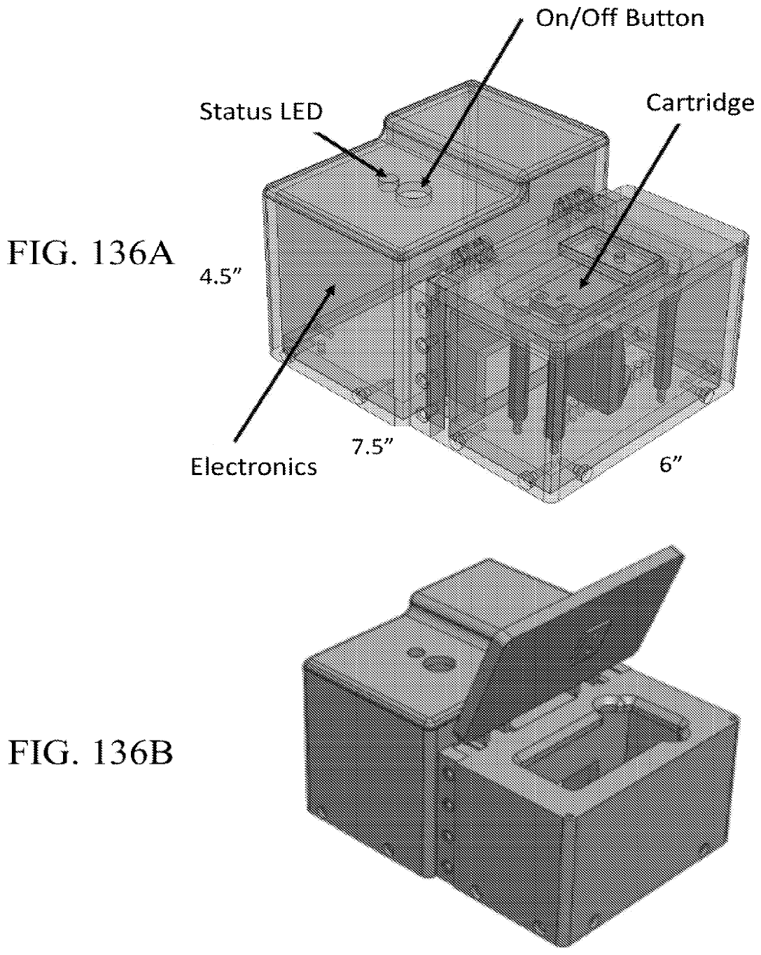

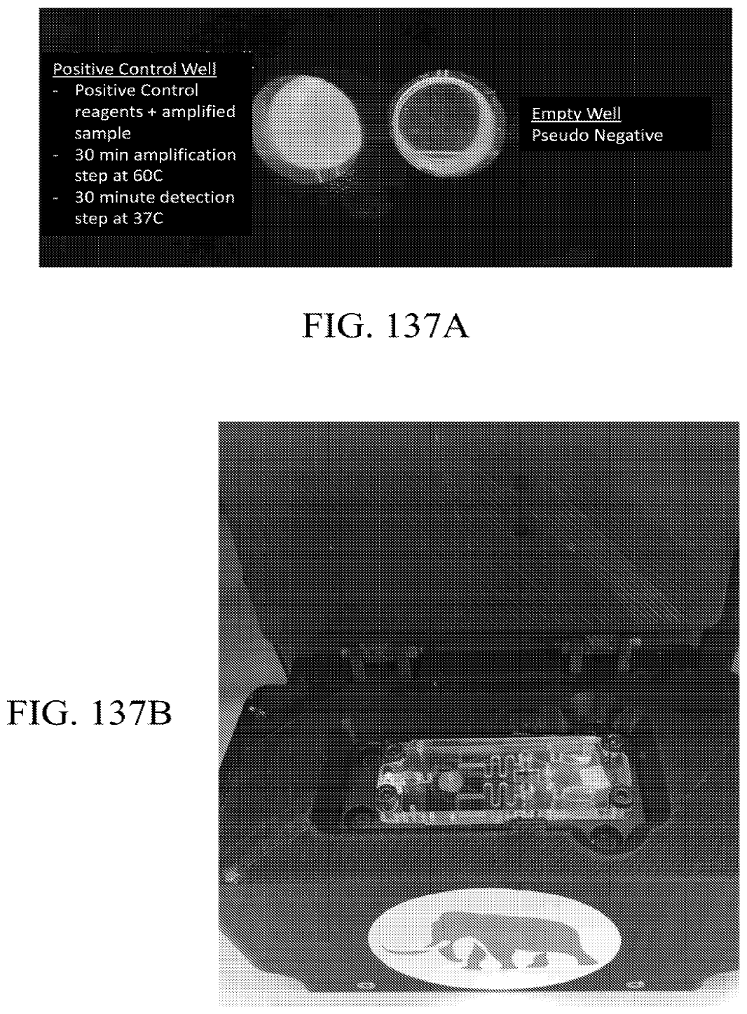

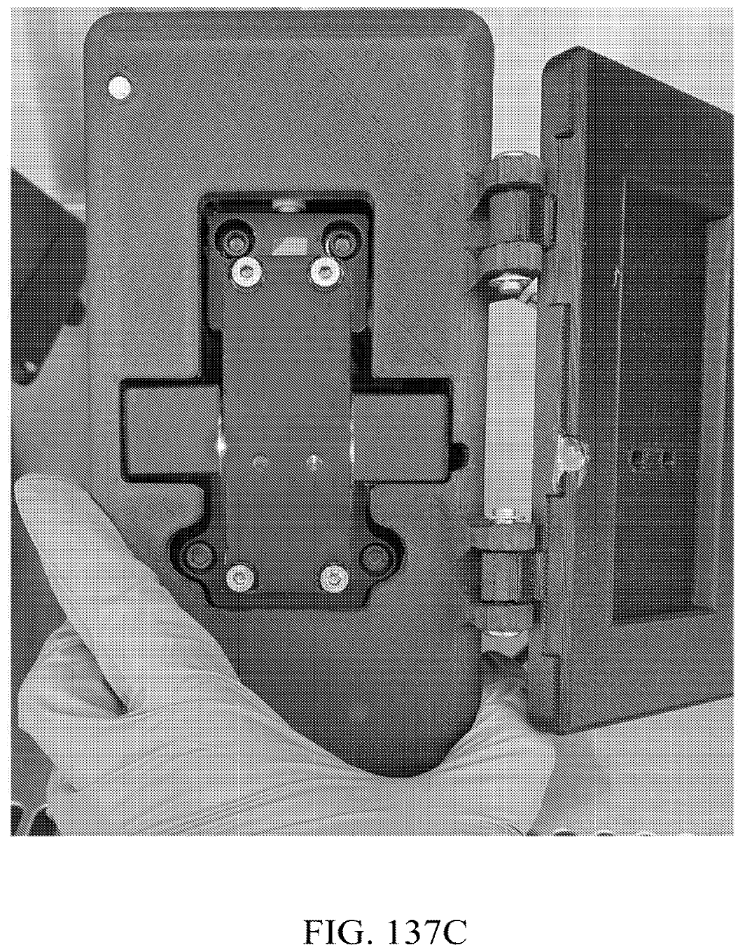

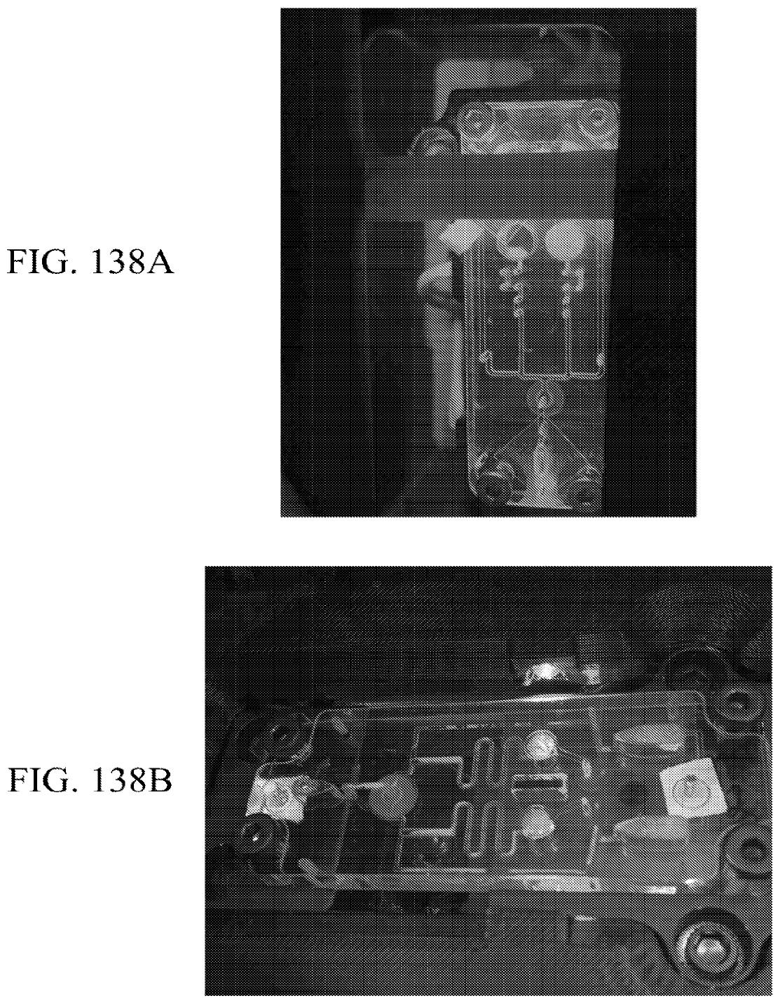

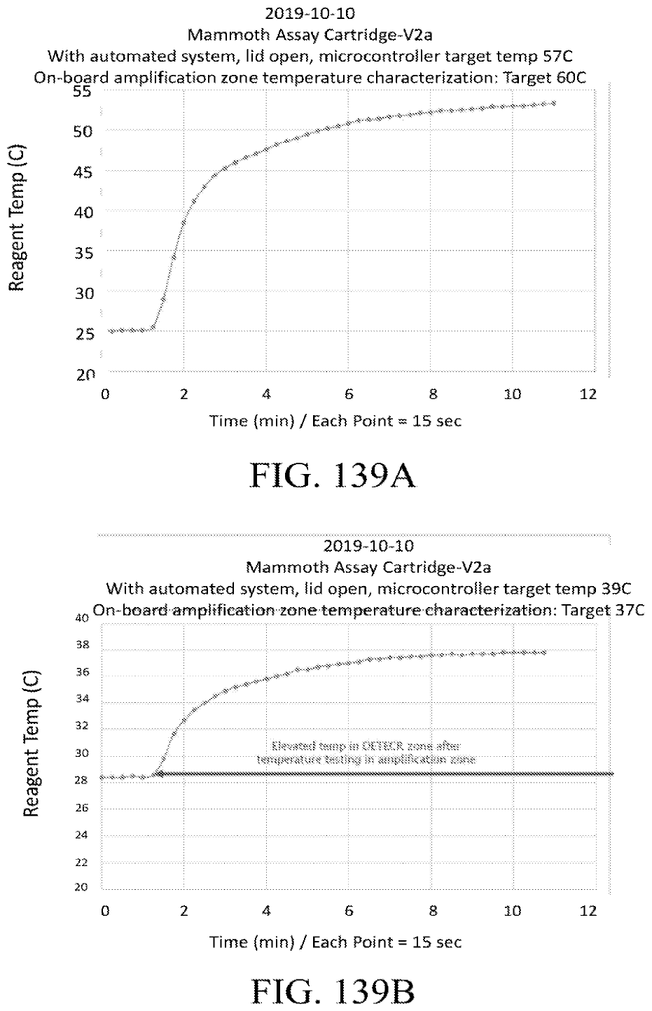

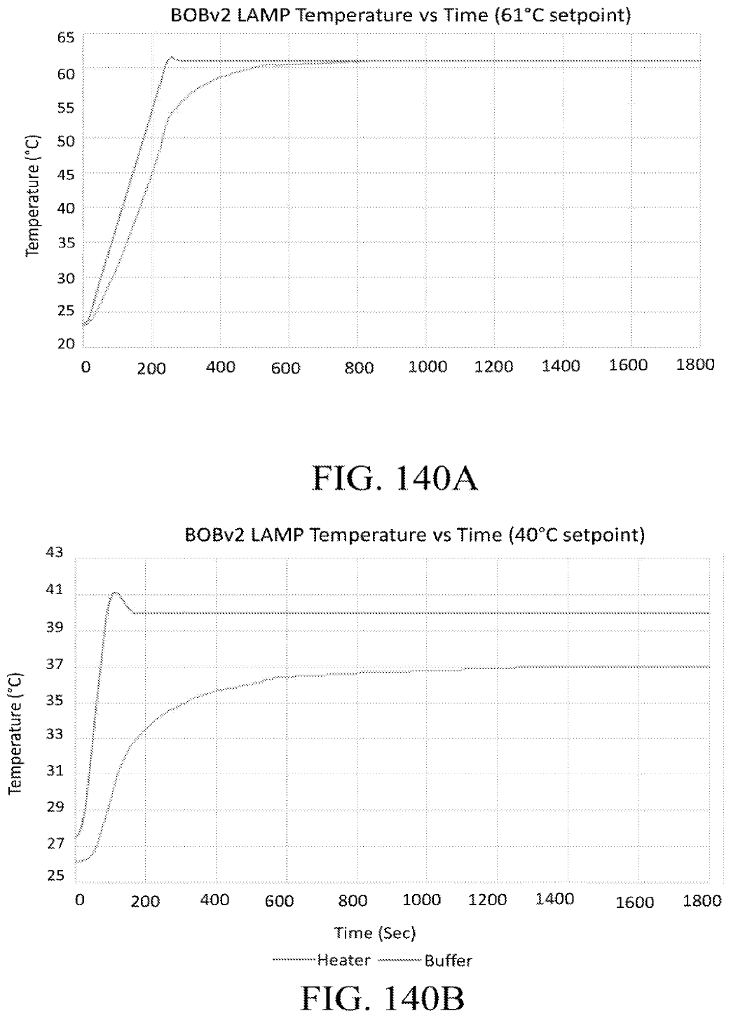

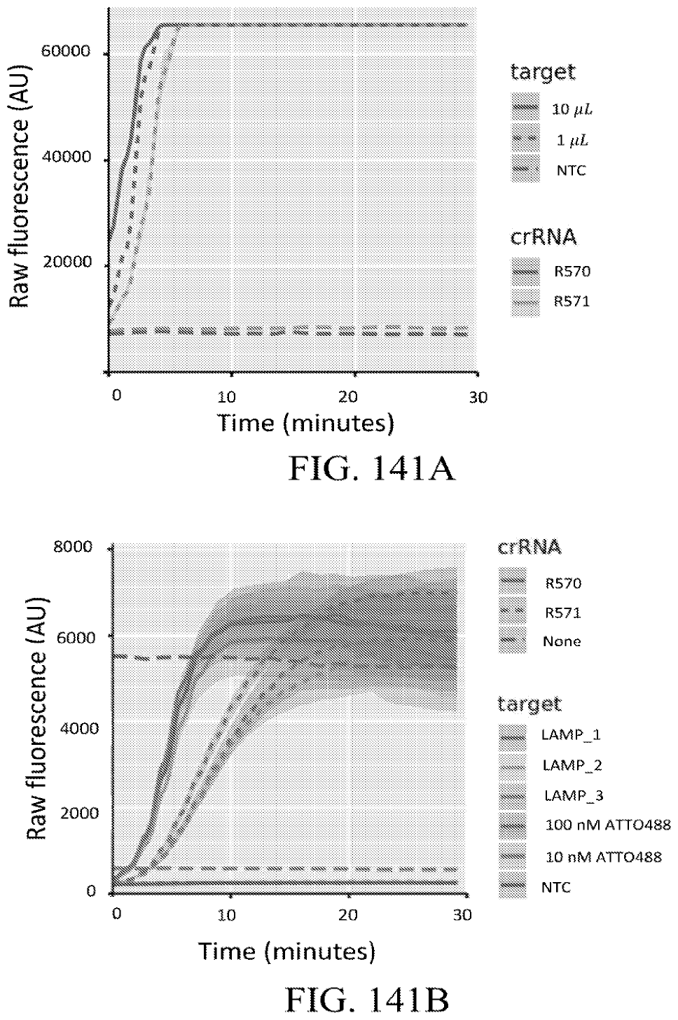

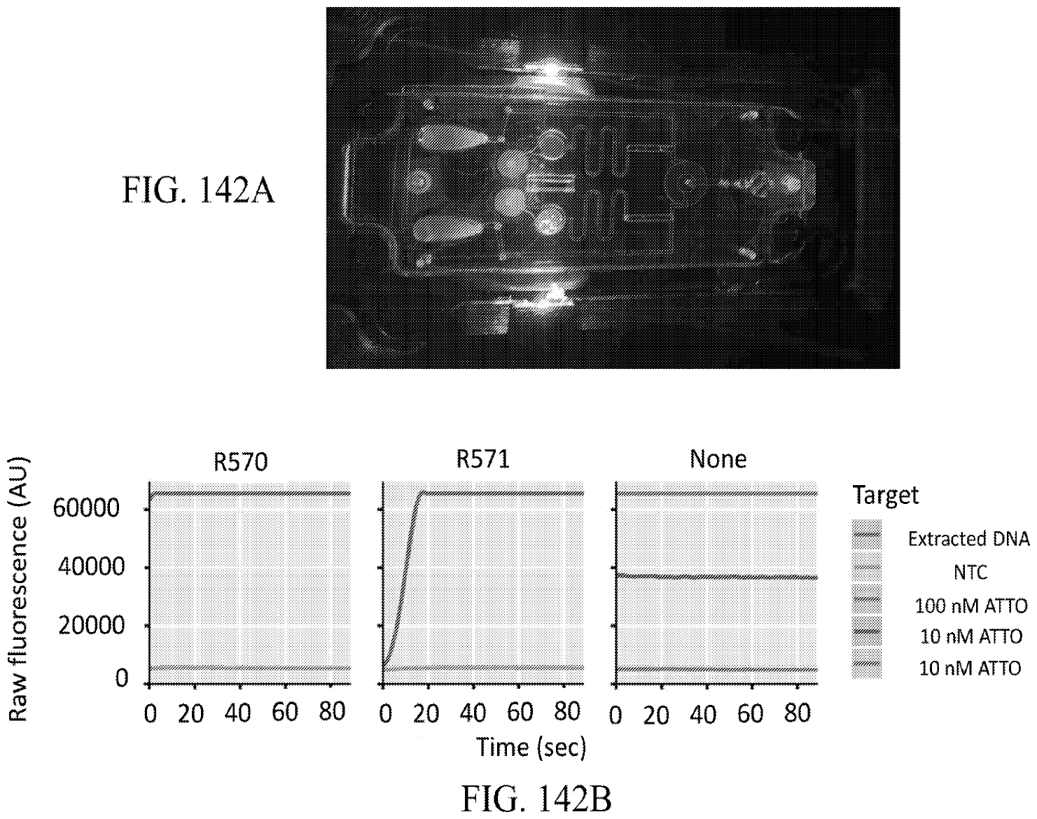

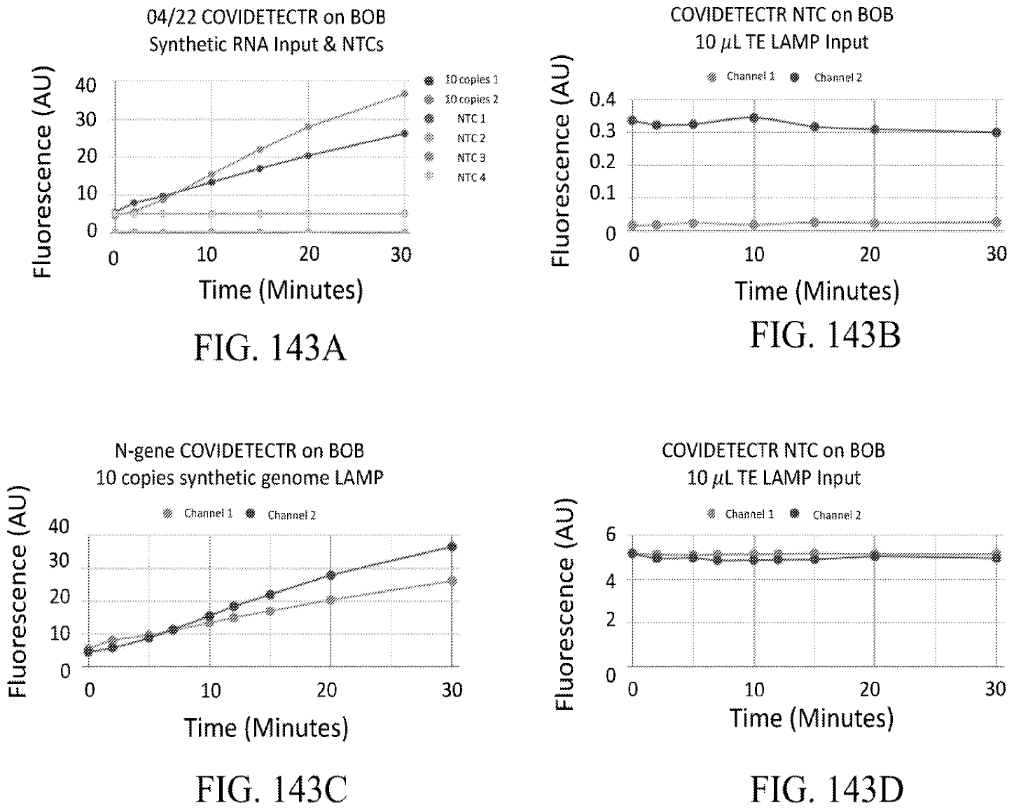

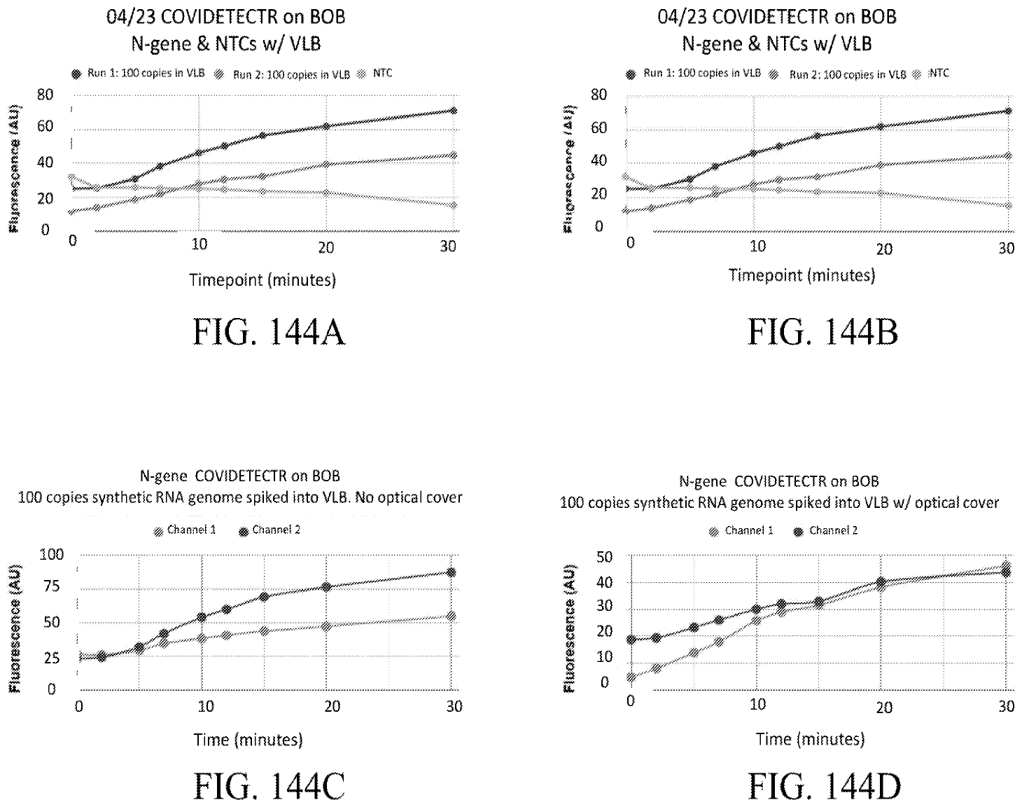

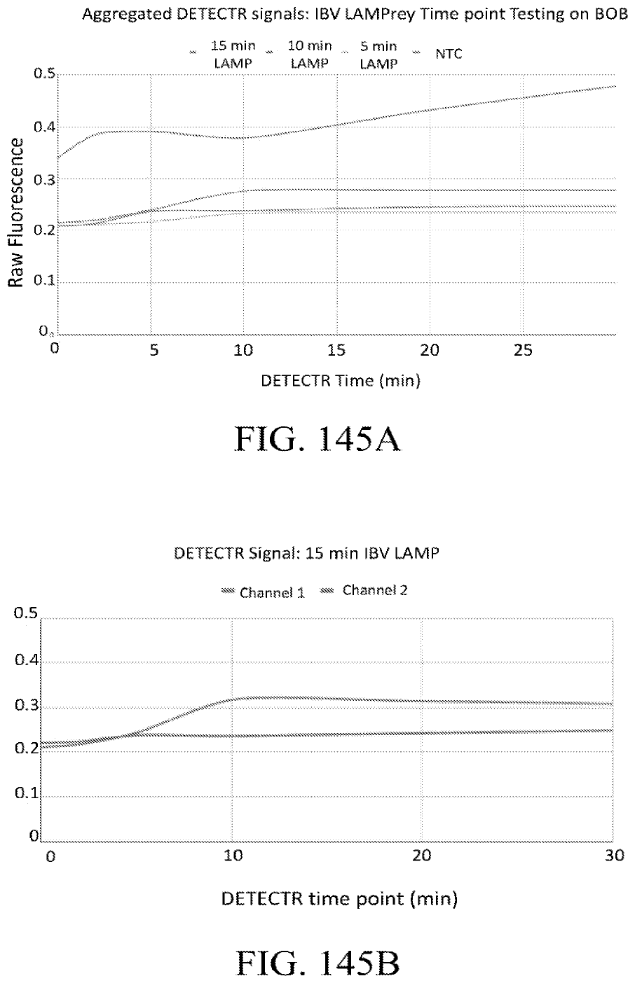

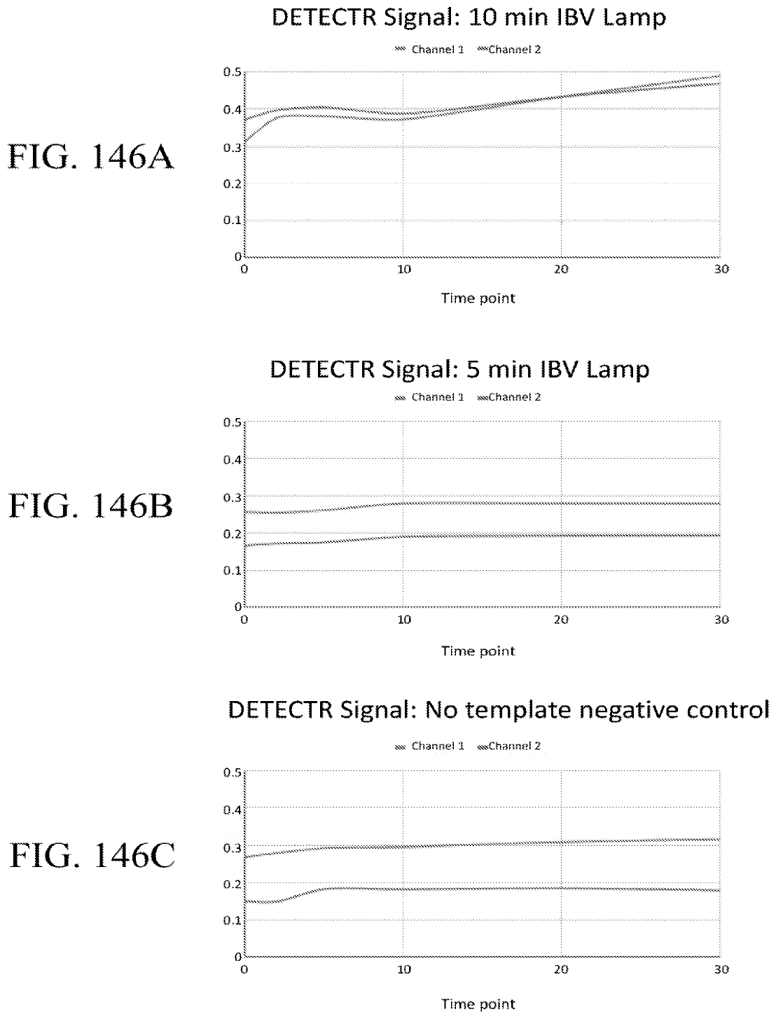

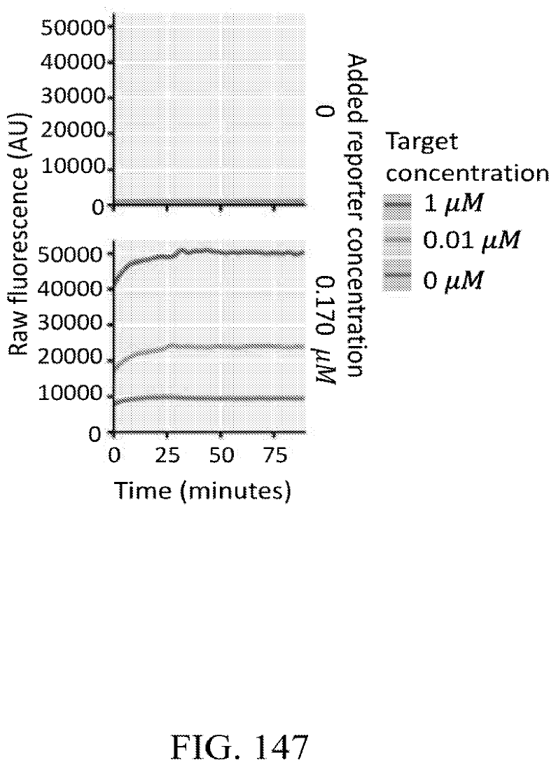

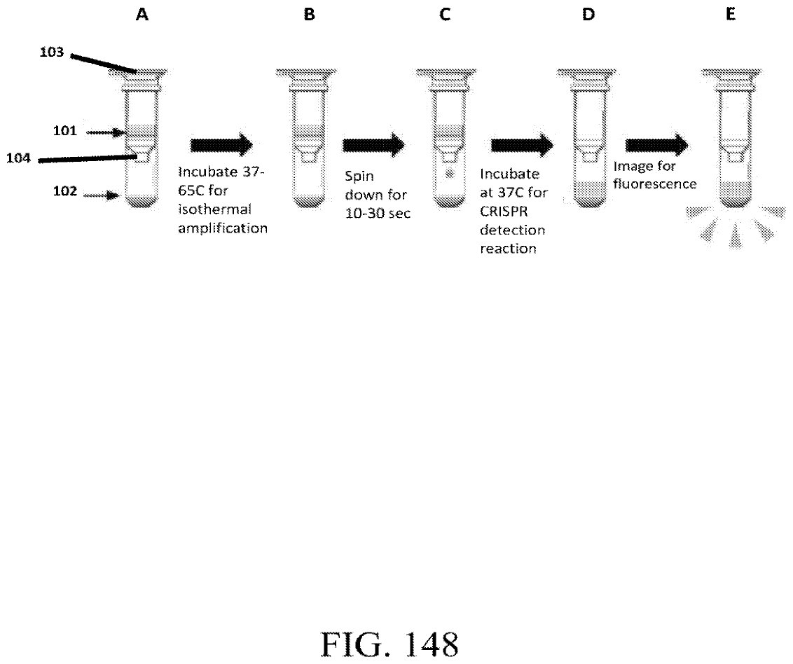

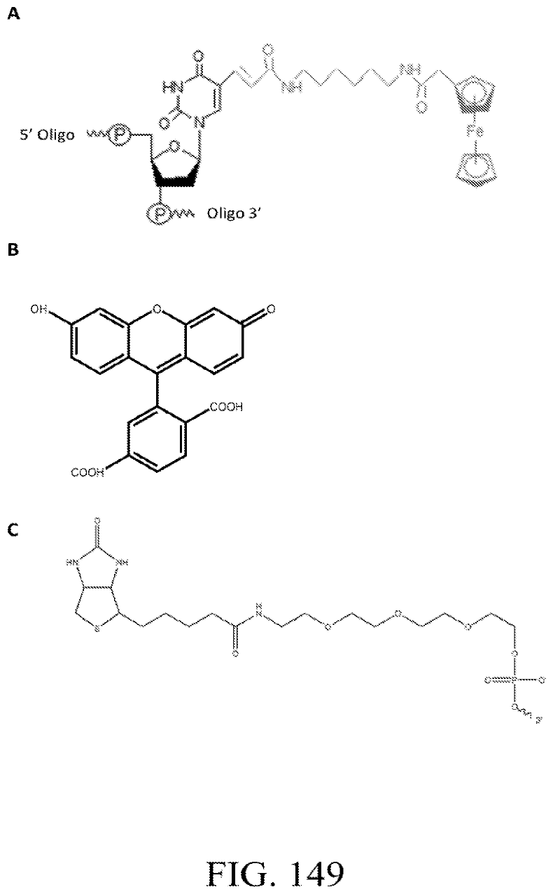

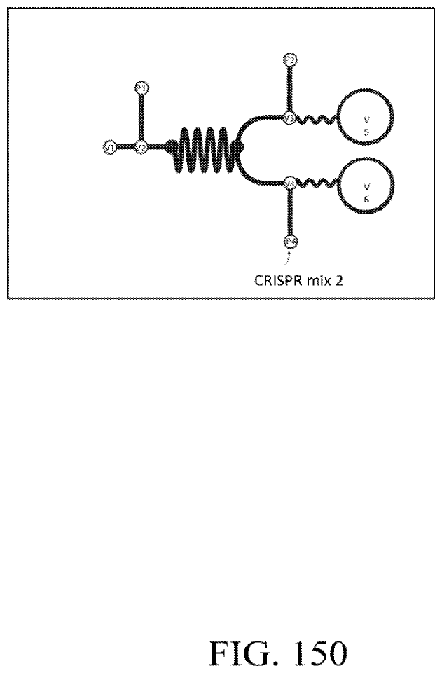

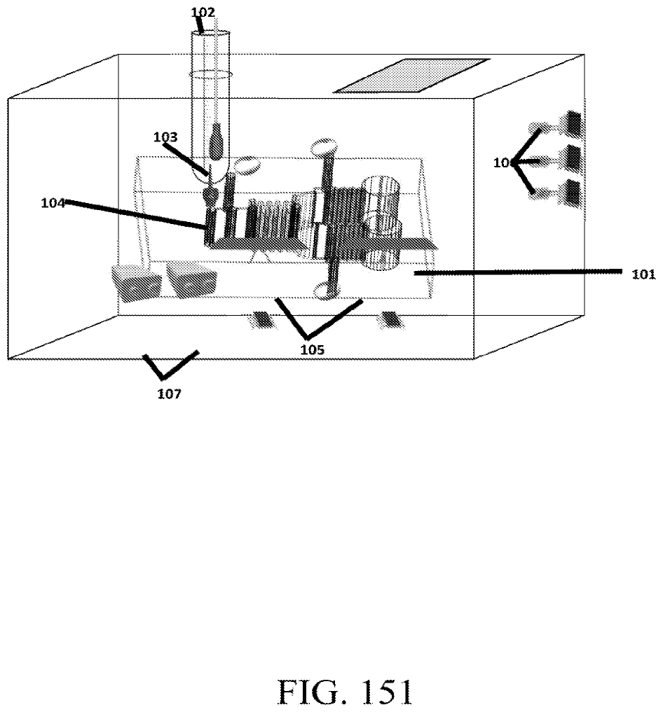

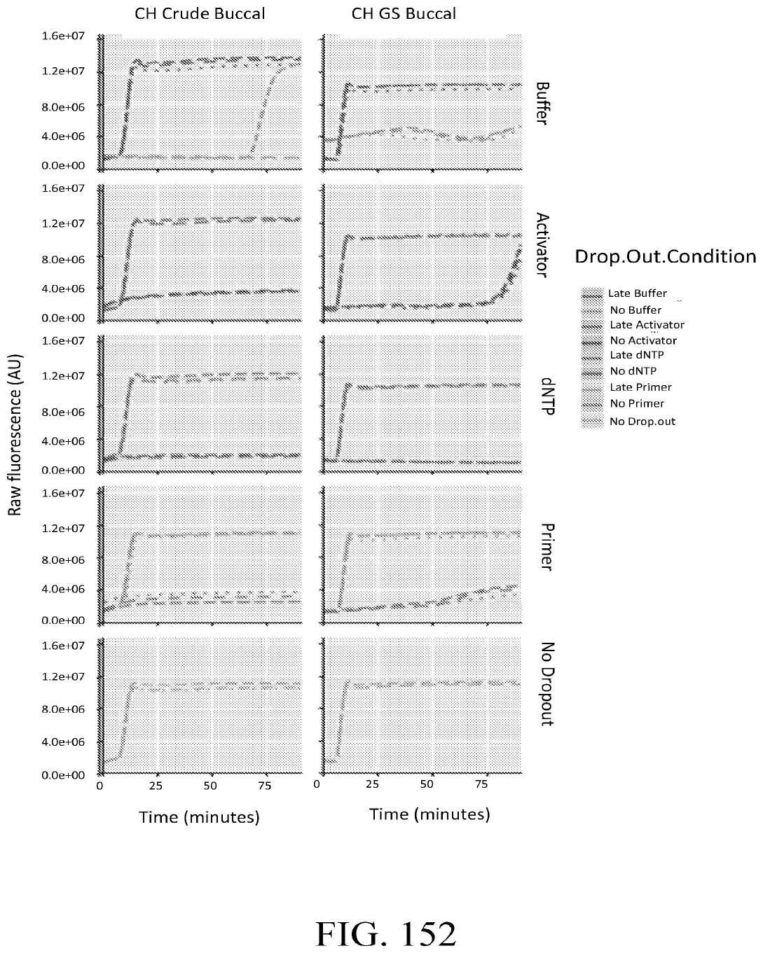

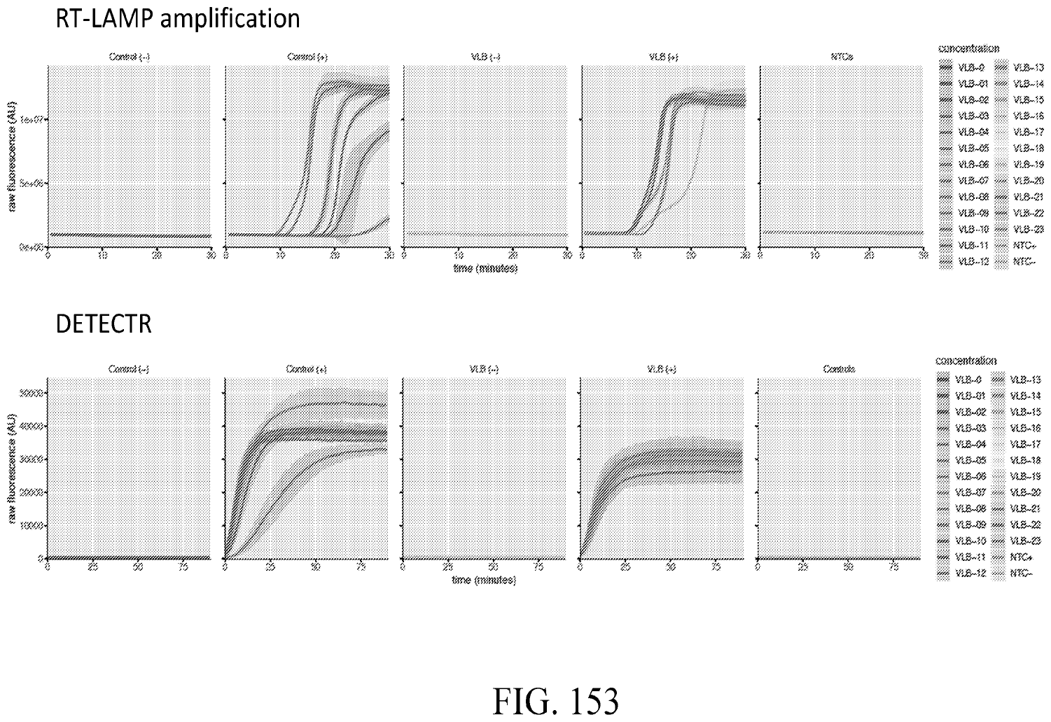

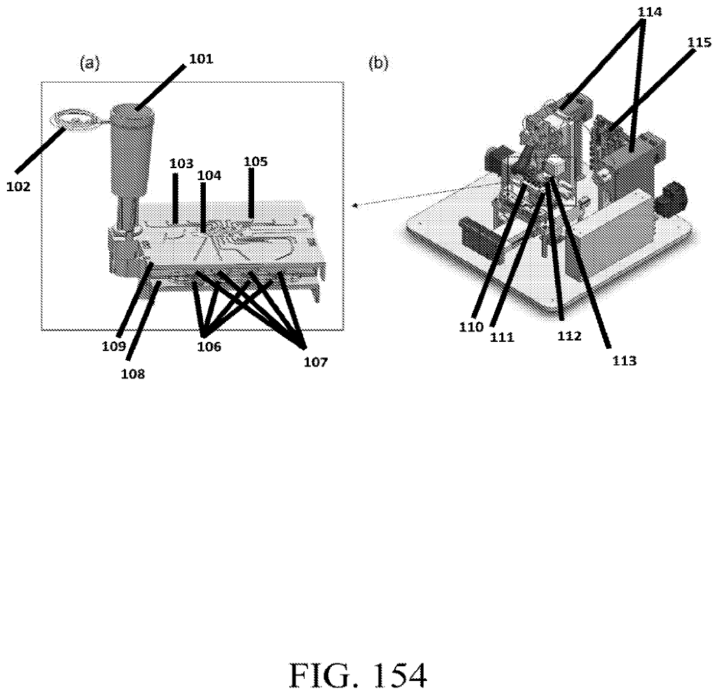

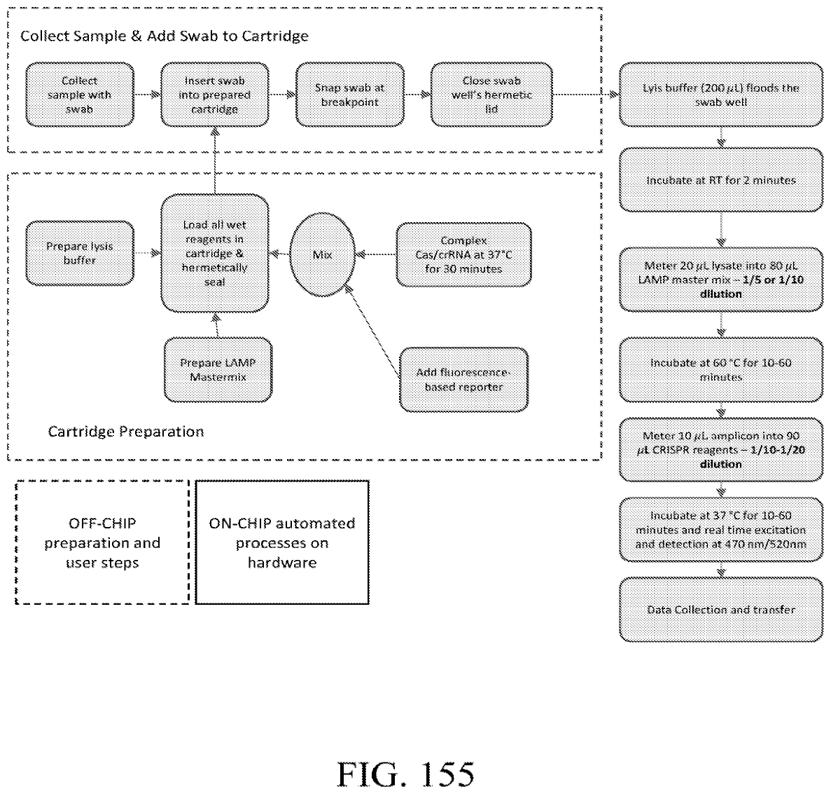

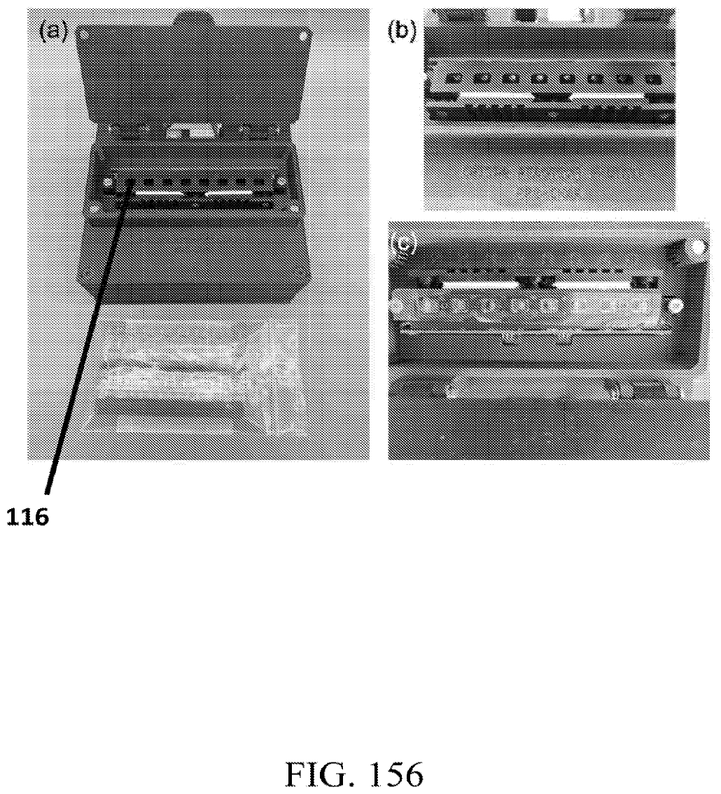

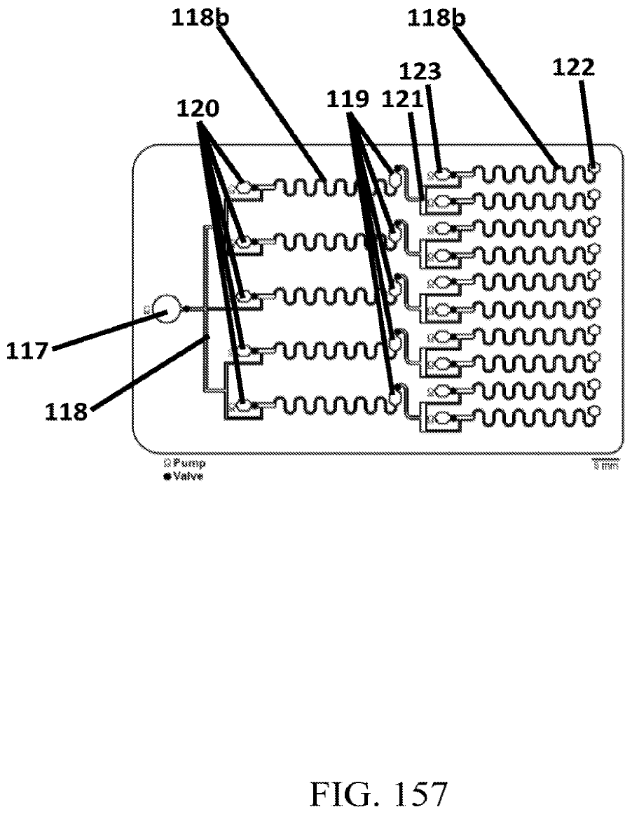

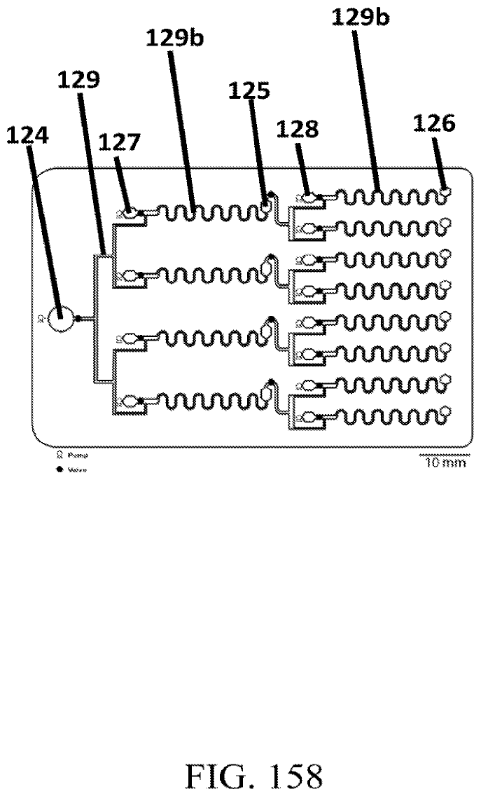

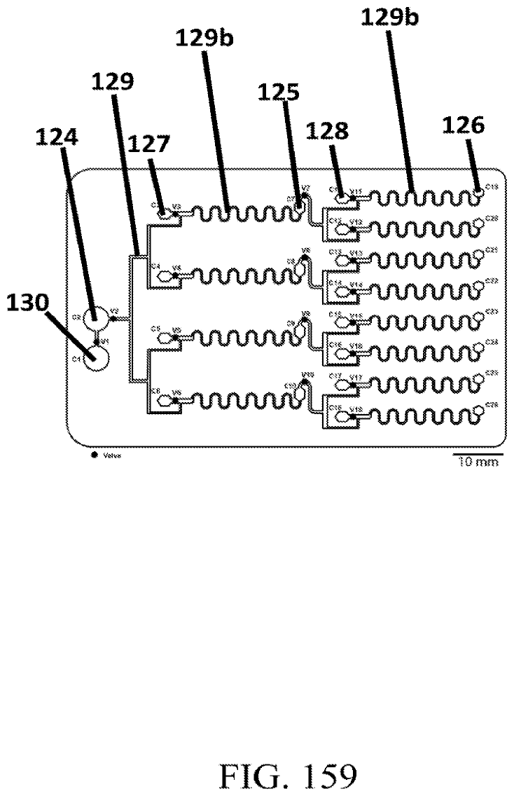

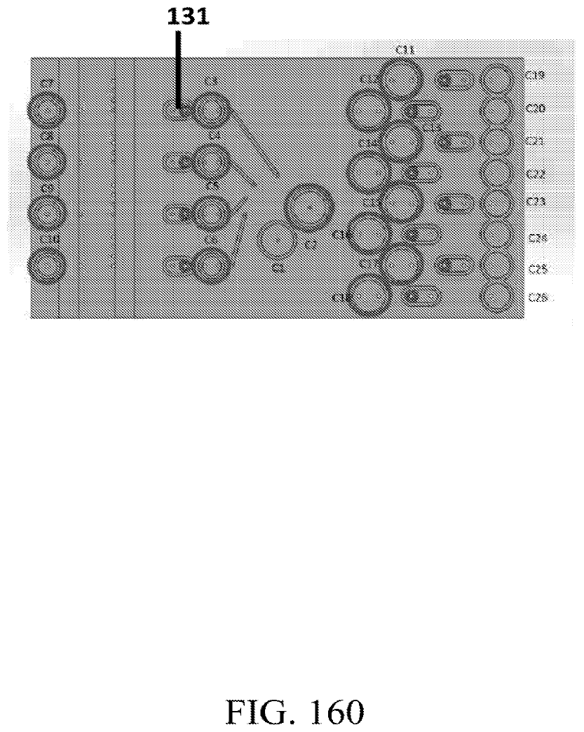

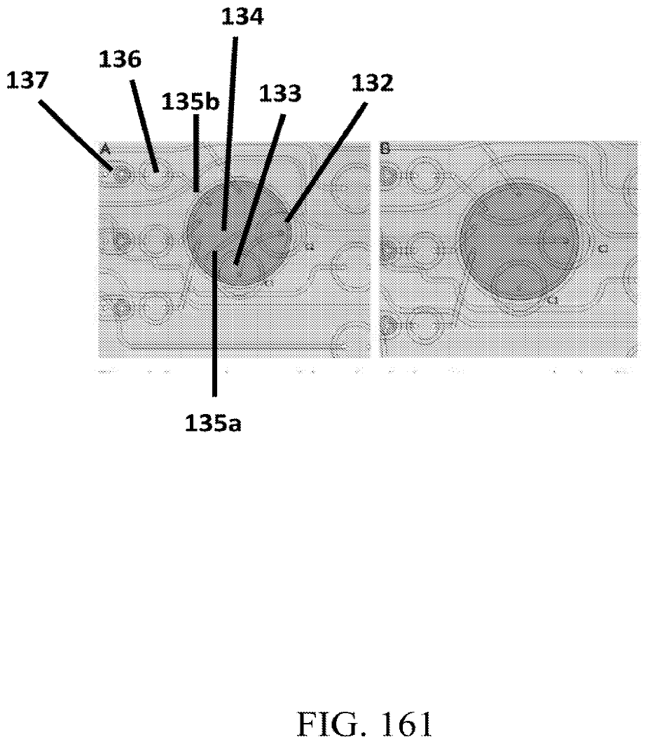

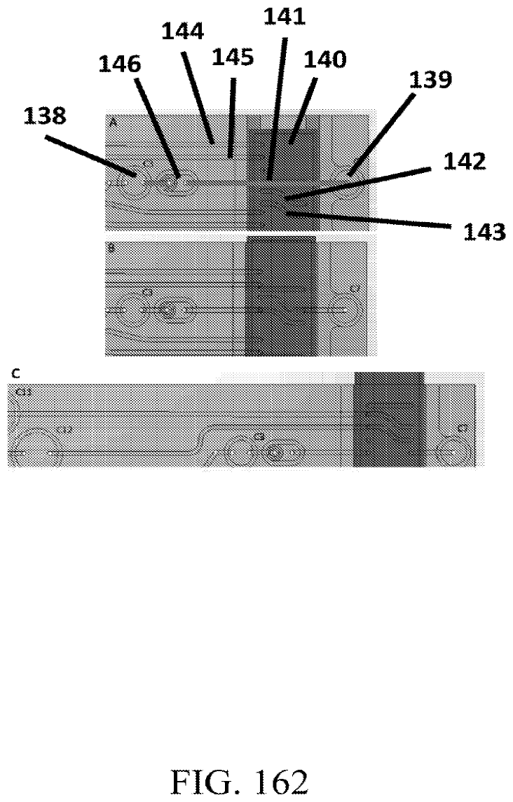

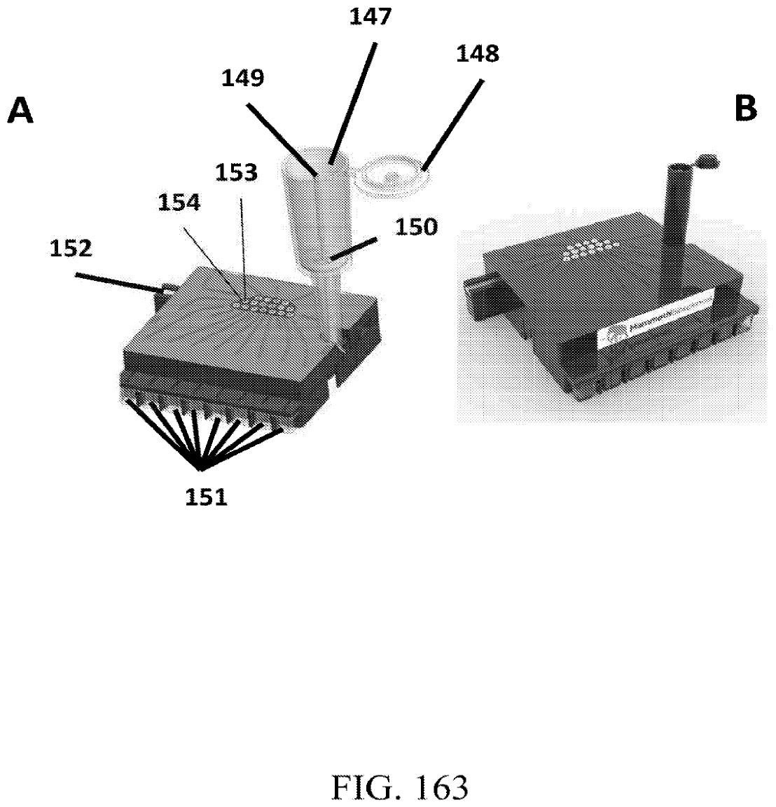

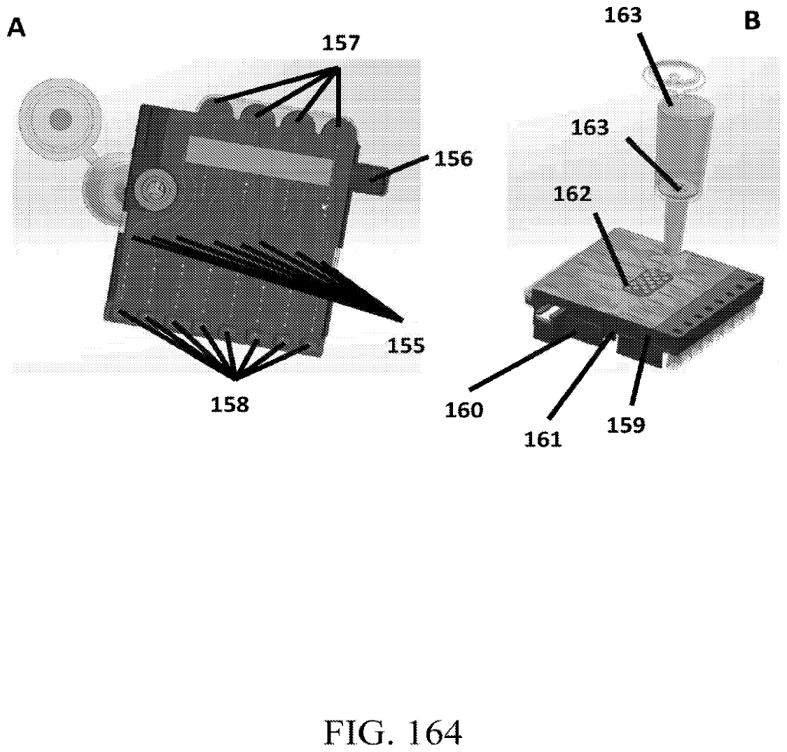

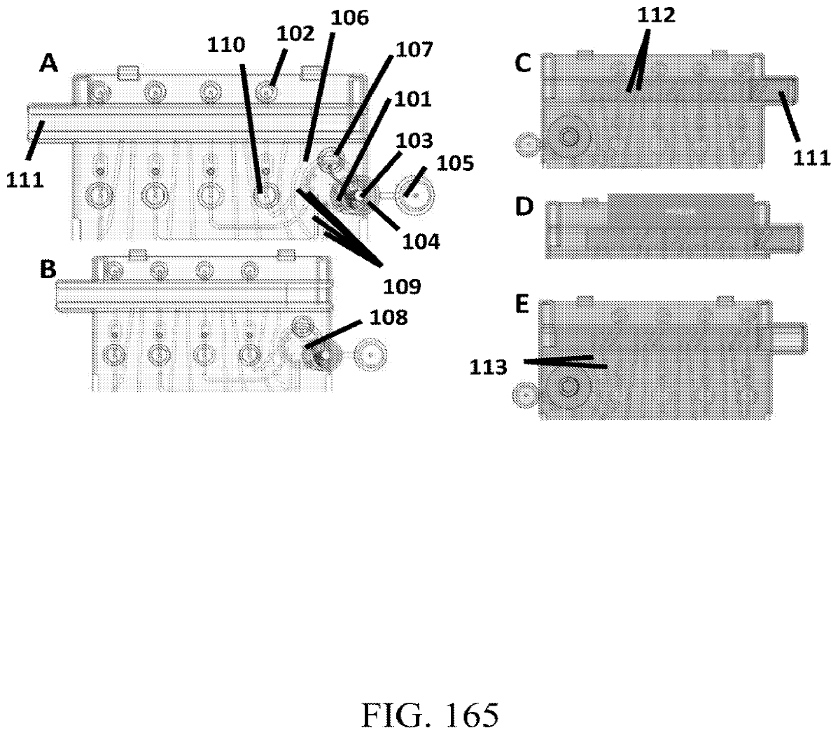

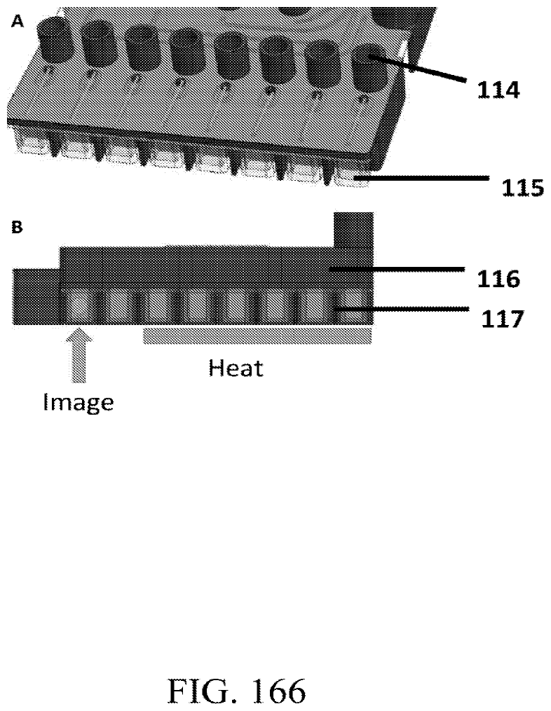

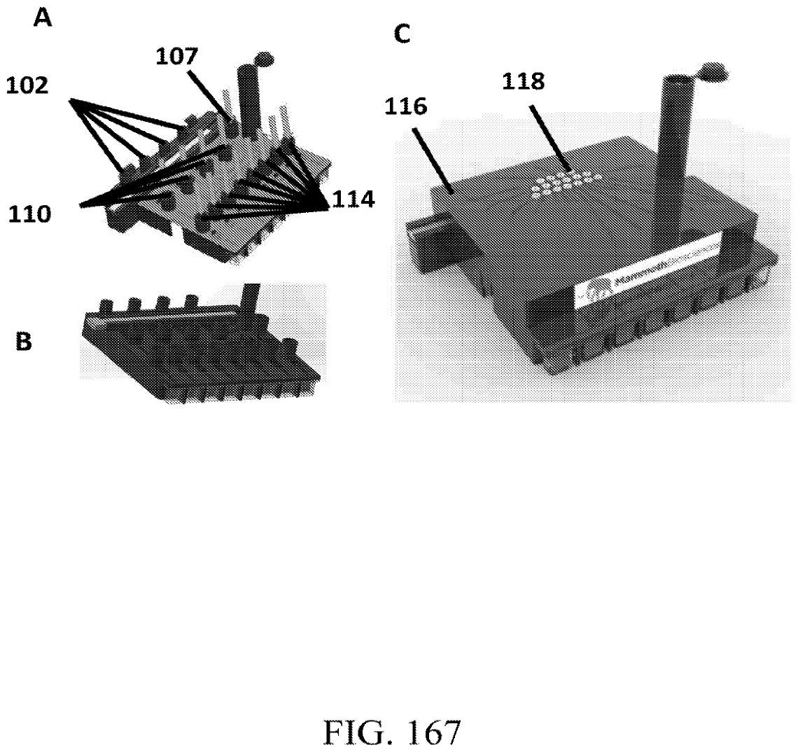

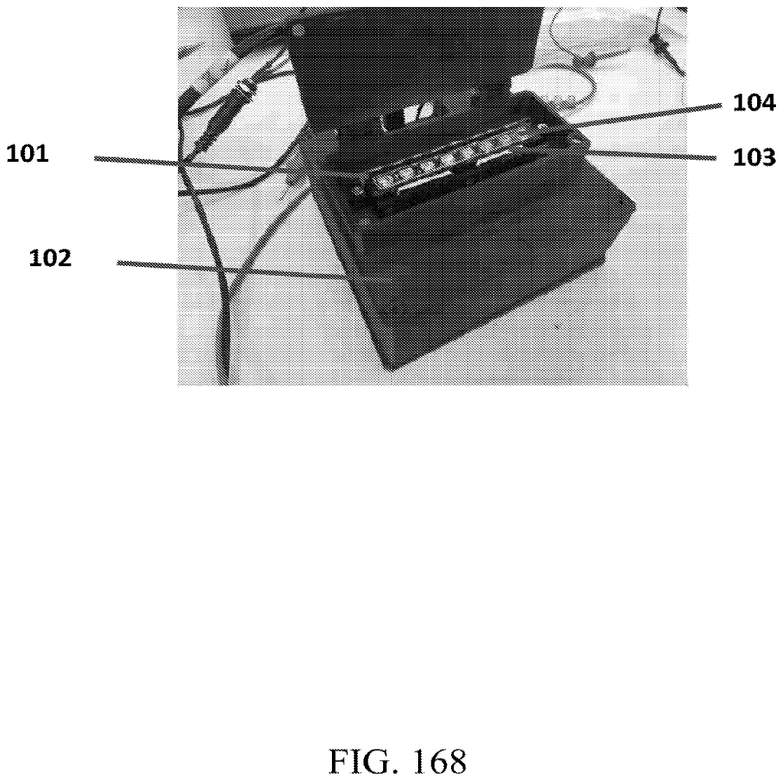

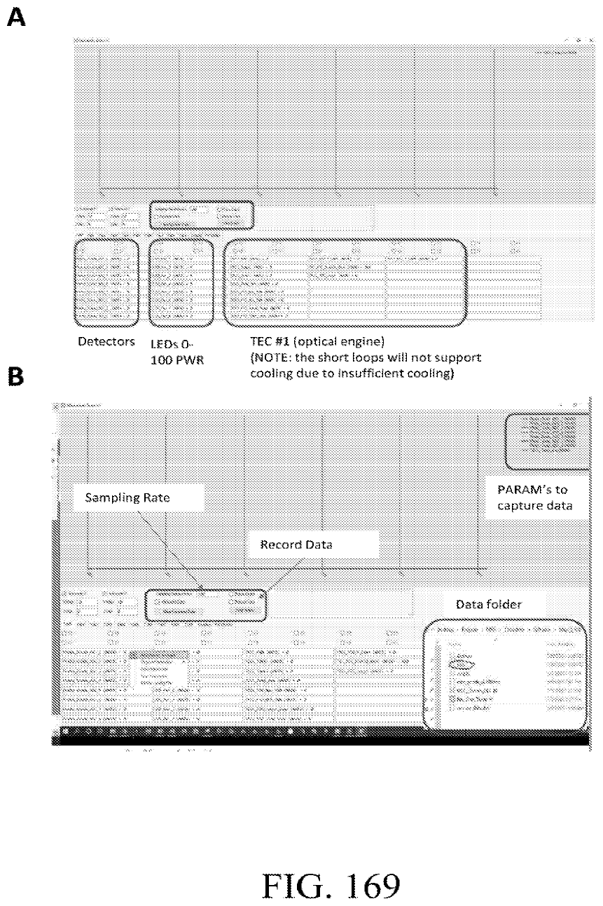

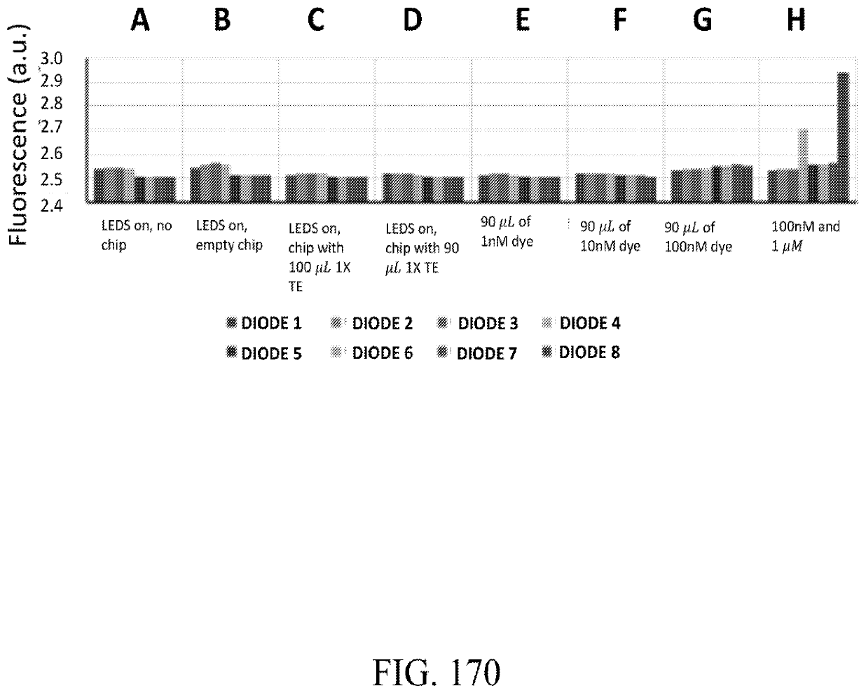

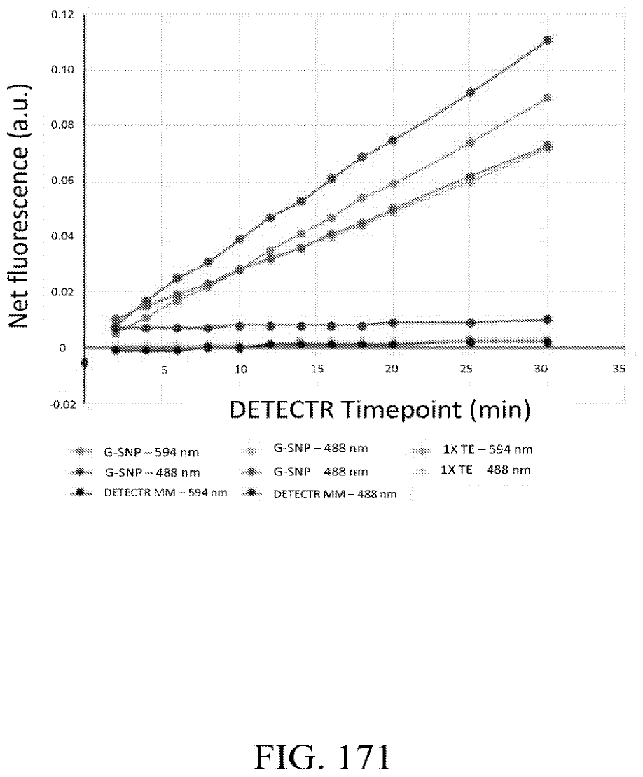

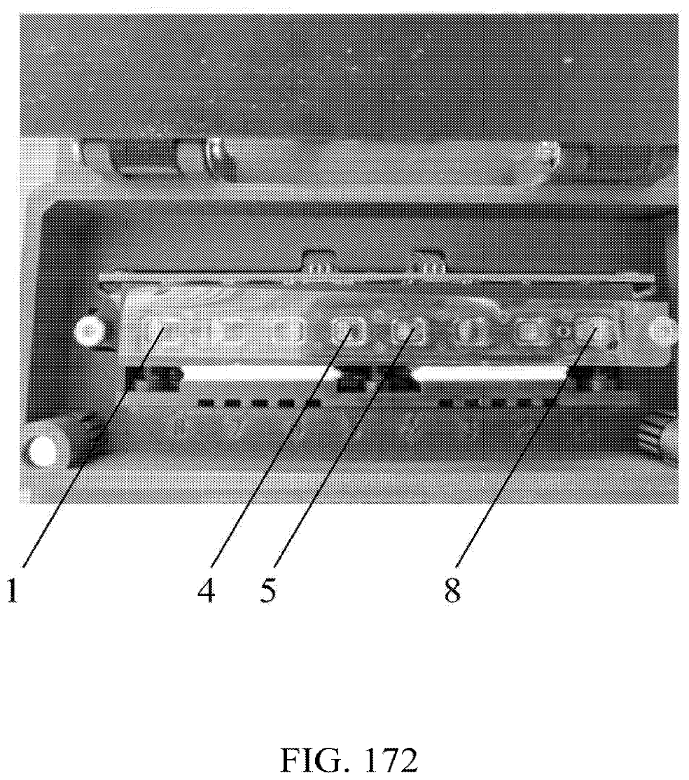

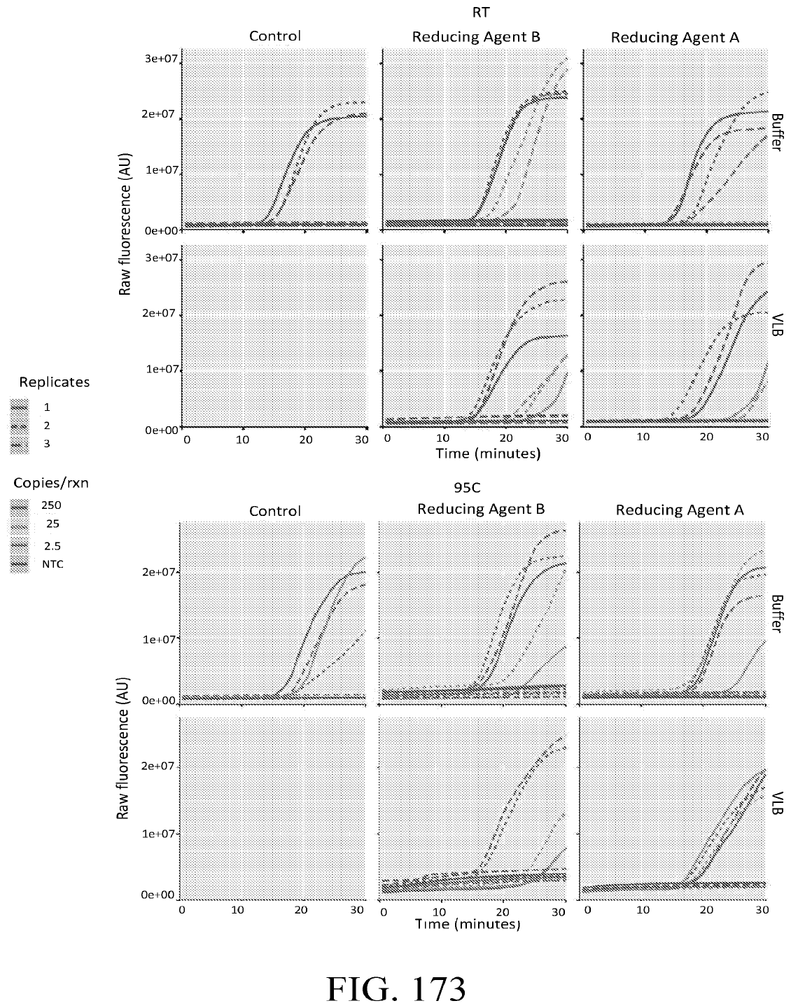

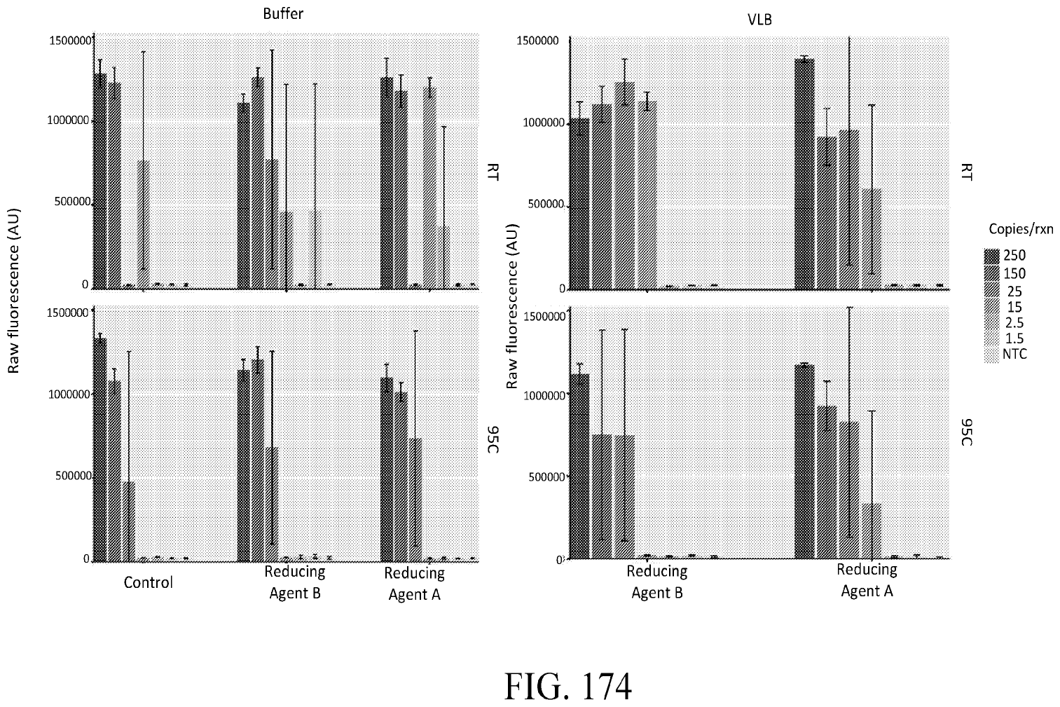

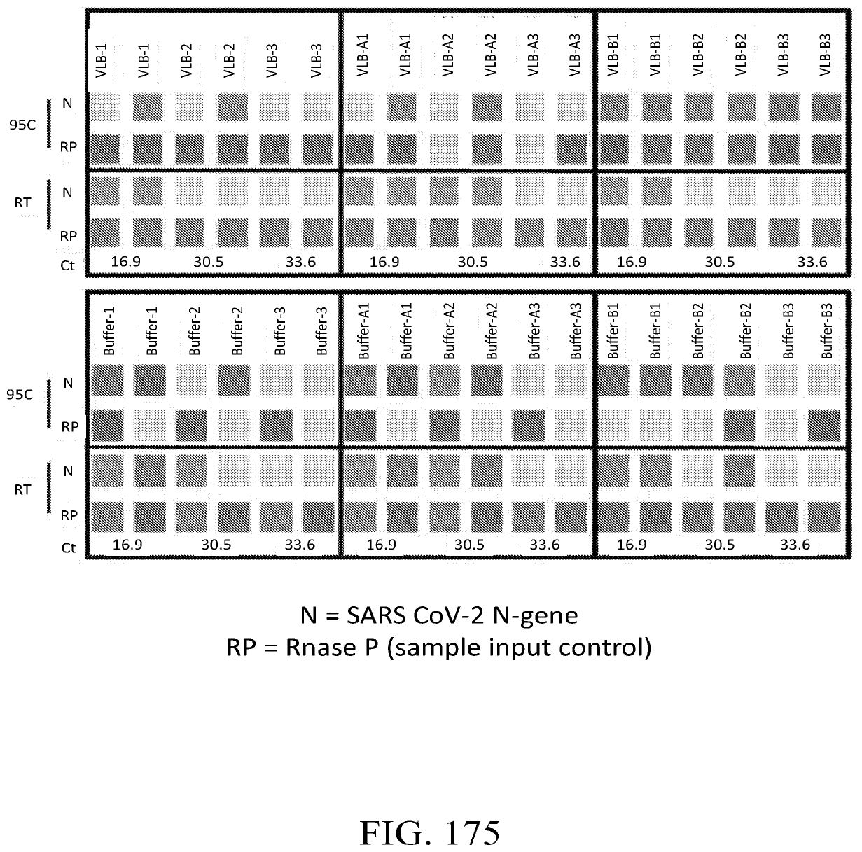

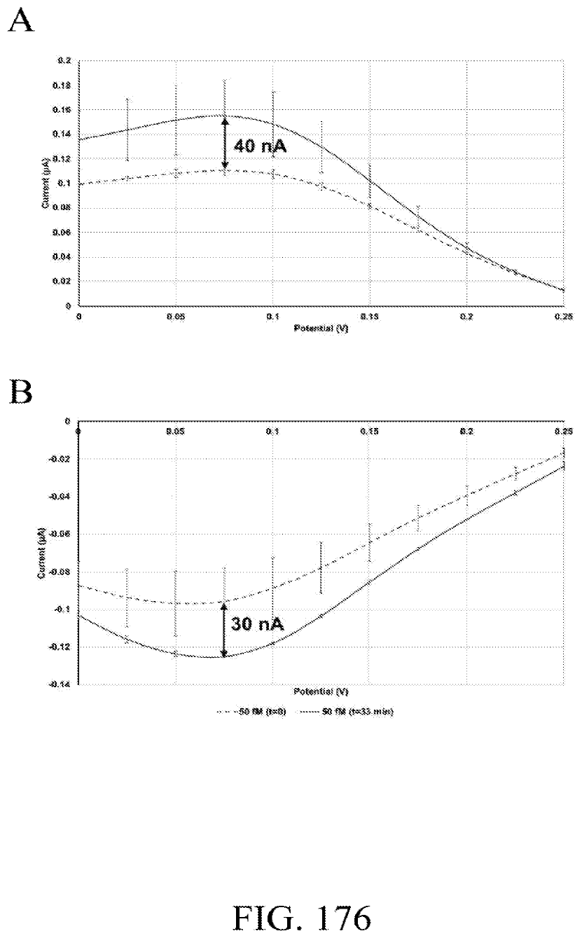

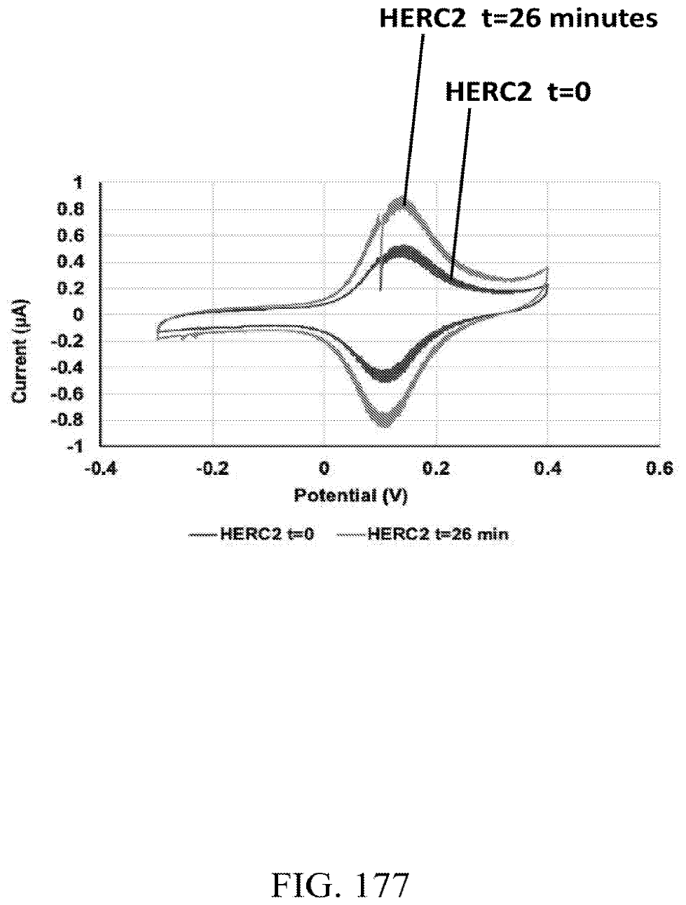

FIG. 20A shows a schematic of the workflow including providing DNA/RNA, LAMP/RT-LAMP, and Cas12a detection.

FIG. 20B shows the time to result of a LAMP/RT-LAMP reaction for RNase P POP7 at different temperatures, as quantified by fluorescence.

FIG. 20C shows three graphs demonstrating Cas12a specific detection at 37° C. of LAMP/RT-LAMP amplicon from the 68° C. temperature reaction.

FIG. 21 shows the specific detection of three different RT-LAMP amplicons for Influenza A virus. At left is a schematic of the workflow including providing DNA/RNA, LAMP/RT-LAMP, and Cas12a detection. At right are graphs showing the results of Cas12a detection as measured by fluorescence over time for each tested condition.

FIG. 22 shows the identification of optimal crRNAs for the specific detection of Influenza B (IBV) RT-LAMP amplicons. At left is a schematic of the workflow including providing DNA/RNA, LAMP/RT-LAMP, and Cas12a detection. At right are graphs showing the results of Cas12a detection as measured by fluorescence over time for each tested condition (IAV is influenza A virus, IBV is influenza B virus, NTC is no template control).

FIG. 23 shows the results of the 1% agarose gel with bands showing the products of the RT-LAMP reaction.

FIGS. 24A-24C show Cas12a discrimination between amplicons from a multiplex RT-LAMP reaction for Influenza A and Influenza B.

FIG. 24A shows a schematic of the workflow including providing viral RNA, multiplexed RT-LAMP, and Cas12a influenza A detection or Cas12a influenza B detection.

FIG. 24B shows Cas12a detection of RT-LAMP amplicons after 30 minute multiplexed RT-LAMP amplification at 60° C.

FIG. 24C shows background subtracted fluorescence at 30 minutes of Cas12a detection at 37° C. of RT-LAMP amplicons for 10,000 viral genome copies of IAV and IBV.

FIG. 25 shows Cas12a discrimination between a triple multiplexed RT-LAMP reaction for Influenza A, Influenza B, and the Mammuthus primigenius (Wooly Mammoth) mitochondria internal amplification control sequence after 30 minutes of multiplexed RT-LAMP amplification at 60° C. At top is a schematic of the worrkflow including providing viral RNA, multiplexed RT-LAMP, and Cas12a influenza A detection or Cas12a influenza B detection or Cas12 internal amplification control detection. At bottom are graphs showing the results of Cas12 detection as measured by fluorescence over time for each tested condition.

FIGS. 26A-26B show schematics of LAMP and RT-LAMP primer designs.

FIG. 26A shows a schematic illustrating the identity of the primers used in LAMP and RT-LAMP. Primers LF and LB are option in some LAMP and RT-LAMP designs, but generally increase the efficiency of the reaction.

FIG. 26B shows a schematic illustrating the position and orientation of the T7 promoter in a variety of LAMP primers.

FIG. 27 shows that a T7 promoter can be included on the F3 or B3 primers (outer primers), or FIP or BIP primers for Influenza A.

FIG. 27A shows a schematic of the workflow including providing DNA/RNA, LAMP/RT-LAMP, in vitro transcription, and Cas13a detection.

FIG. 27B shows the time to result for RT-LAMP reactions for Influenza A using different primer sets, as quantified by fluorescence.

FIG. 27C shows in vitro transcription (IVT) with T7 RNA polymerase of the product of the RT-LAMP reactions for Influenza A using different primer sets at 37° C. for 10 minutes.

FIG. 28 shows the detection of a RT-SIBA amplicon for Influenza A by Cas12. At left is a schematic of the workflow including providing DNA/RNA, SIBA/RT-SIBA, and Cas12a detection. At right is a graph showing Cas12a detection as measured by fluorescence for each of the tested conditions.

FIG. 29 shows the layout of a Milenia commercial strip with a typical reporter.

FIG. 30 shows the layout of a Milenia HybridDetect 1 strip with an amplicon.

FIG. 31 shows the layout of a Milenia HybridDetect 1 strip with a standard Cas reporter.

FIG. 32 shows a modified Cas reporter comprising a DNA linker to biotin-dT (shown as a pink hexagon) bound to a FAM molecule (shown as a green start).

FIG. 33 shows the layout of Milenia HybridDetect strips with the modified Cas reporter.

FIG. 34 shows an example of a single target assay format (to left) and a multiplexed assay format (to right).

FIG. 35 shows another variation of an assay prior to use (top), an assay with a positive result (middle left), an assay with a negative result (middle right), and a failed test (bottom).

FIG. 36 shows one design of a tethered lateral flow Cas reporter.

FIG. 37 shows a workflow for CRISPR diagnostics using the tethered cleavage reporter using magnetic beads.

FIG. 38 shows a schematic for an enzyme-reporter system that is filtered by streptavidin-biotin before reaching the reaction chamber.

FIG. 39 shows an invertase-nucleic acid used for the detection of a target nucleic acid. The invertase-nucleic acid, immobilized on a magnetic bead, is added to a sample reaction containing Cas protein, guide RNA, and a target nucleic acid. Target recognition activates the Cas protein to cleave the nucleic acid of the invertase-nucleic acid, liberating the invertase enzyme from the immobilized magnetic bead. This solution is either be transferred to the “reaction mix”, which contains sucrose and the DNS reagent and changes color from yellow to red when the invertase converts sucrose to glucose or is can be transferred to a hand-held glucometer device for a digital readout.

FIG. 40 shows one layout for a two-pot DETECTR assay. In this layout a swab collection cap seals a swab reservoir chamber. Clockwise to the swab reservoir chamber is a chamber holding the amplification reaction mix. Clockwise to the chamber holding the amplification reaction mix is a chamber holding the DETECTR reaction mix. Clockwise to this is the detection area. Clockwise to the detection area is the pH balance well. A cartridge wells cap is shown and seals all the wells containing the various reagent mixtures. The cartridge itself is shown as a square layer at the bottom of the schematic. To the right is a diagram of the instrument pipers pump which drives the fluidics in each chamber/well and is connected to the entire cartridge. Below the cartridge is a rotary valve that interfaces with the instrument.

FIG. 41 shows one workflow of the various reactions in the two-pot DETECTR assay of FIG. 40. First, as shown in the top left diagram, a swab may be inserted into the 200 ul swab chamber and mixed. In the middle left diagram, the valve is rotated clockwise to the “swab chamber position” and 1 uL of sample is picked up. In the lower left diagram, the valve is rotated clockwise to the “amplification reaction mix” position and the 1 ul of sample is dispensed and mixed. In the top right diagram, 2 uL of sample is aspirated from the “amplification reaction mix”. In the top middle diagram, the valve is roated clockwise to the “DETECTR” position, the sample is dispensed and mixed, and 20 ul of the sample is aspirated. Finally, in the bottom right diagram, the valve is rotated clockwise to the detection area position and 20 ul of the sample is dispensed.

FIG. 42 shows a modification of the workflow shown in FIG. 41 that is also consistent with the methods and systems of the present disclosure. At left is the diagram shown at the top right of FIG. 41. At right is the modifed diagram in which there is a first amplification chamber counterclockwise to the swab lysis chamber and a second amplification chamber clockwise to the swab lysis chamber. Additionally, clockwise to amplification chamber #2 are two sets, or “duplex”, DETECTR chambers labeled “Duplex DETECTR Chambers #2” and “Duplex DETECTR Chambers #1”, respectively.

FIG. 43 shows breakdown of the workflow for the modified layout shown in FIG. 42. Specifically, from the swab lysis chamber, which holds 200 ul of sample, 20 ul of the sample can be moved to amplification chmaber #1 and 20 ul of the sample can be moved to amplification chamber #2. After amplification in amplification chamber #1, 20 ul of the sample can be moved to Duplex DETECTR Chambers #1a and 20 ul of the sample can be moved to Duplex DETECTR Chambers #1b. Additionally, after amplification in amplification chamber #2, 20 ul of the sample can be moved to Duplex DETECTR Chambers #2a and 20 ul of the sample can be moved to Duplex DETECTR Chambers #2b.

FIG. 44 shows the modifications to the cartridge illustrated in FIG. 43 and FIG. 42.

FIG. 45 shows a top down view of the cartridge of FIG. 44. This layout and workflow has a replicate in comparison to the layout and workflow of FIGS. 40-41.

FIG. 46 shows a layout for a two-pot DETECTR assay. Shown at top is a pneumatic pump, which interfaces with the cartridge. Shown at middle is a top down view of the cartridge showing a top layer with reservoirs. Shown at bottom is a sliding valve containing the sample and arrows pointing to the lysis chamber at left, following by amplification chambers to the right, and DETECT chambers further to the right.

FIG. 47 shows a comparison of the DETECTR assays disclosed herein to the gold standard PCR-based method of detecting a target nucleic acid. Shown is a flow chart showing a gradient of sample prep evaluation from crude (left) to pure (right). Sample prep steps that take a crude sample to a pure sample include lysis, binding, washing, and eluting. DETECTR assays disclosed herein may only need the sample prep step of lysis, yielding a crude sample. On the other hand, PCR-based methods can require lysis, binding, washing, and elution, yielding a very pure sample.

FIGS. 48A-48C show Cas13a detection of target RT-LAMP DNA amplicon.

FIG. 48A shows a schematic of the workflow including providing DNA/RNA, LAMP/RT-LAMP, and Cas13a detection.

FIG. 48B shows Cas13a specific detection of target RT-LAMP DNA amplicon with a first primer set as measured by background subtracted fluorescence on the y-axis.

FIG. 48C shows Cas13a specific detection of target RT-LAMP DNA amplicon with a second primer set as measured by background subtracted fluorescence on the y-axis.

FIG. 49A shows a Cas13 detection assay using 2.5 nM RNA, single-stranded DNA (ssDNA), or double-stranded (dsDNA) as target nucleic acids, where detection was measured by fluorescence for each of the target nucleic acid tested.

FIG. 49B shows Cas12 detection assay using 2.5 nM RNA, ssDNA, and dsDNA as target nucleic acids, where detection was measured by fluorescence for each of the target nucleic acid tested.

FIG. 49C shows the performance of Cas13 and Cas12 on target RNA, target ssDNA, and target dsDNA at various concentrations, where detection was measured by fluorescence for each of the target nucleic tested.

FIG. 50 shows an LbuCas13a detection assay using 2.5 nM target ssDNA with 170 nM of various reporter substrates, wherein detection was measured by fluorescence for each of the reporter substrates tested.

FIG. 51A shows the results of Cas13 detection assays for LbuCas13a (SEQ ID NO: 131) and LwaCas13a (SEQ ID NO: 137) using 10 nM or 0 nM of target RNA, where detection was measured by fluorescence resulting from cleavage of reporters over time.

FIG. 51B shows the results of Cas13 detection assays for LbuCas13a (SEQ ID NO: 131) and LwaCas13a (SEQ ID NO: 137) using 10 nM or 0 nM of target ssDNA, where detection was measured by fluorescence resulting from cleavage of reporters over time.

FIG. 52 shows LbuCas13a (SEQ ID NO: 131) detection assay using 1 nM target RNA (at left) or target ssDNA (at right) in buffers with various pH values ranging from 6.8 to 8.2.

FIG. 53A shows guide RNAs (gRNAs) tiled along a target sequence at 1 nucleotide intervals.

FIG. 53B shows LbuCas13a (SEQ ID NO: 131) detection assays using 0.1 nM RNA or 2 nM target ssDNA with gRNAs tiled at 1 nucleotide intervals and an off-target gRNA.

FIG. 53C shows data from FIG. 97B ranked by performance of target ssDNA.

FIG. 53D shows performance of gRNAs for each nucleotide on a 3′ end of a target RNA.

FIG. 53E shows performance of gRNAs for each nucleotide on a 3′ end of a target ssDNA.

FIG. 54A shows LbuCas13a detection assays using 1 μL of target DNA amplicon from various LAMP isothermal nucleic acid amplification reactions.

FIG. 54B shows LbuCas13a (SEQ ID NO: 131) detection assays using various amounts of PCR reaction as a target DNA.

FIG. 55 shows a pneumatic valve device layout for a DETECTR assay.

FIG. 55A shows a schematic of a pneumatic valve device. A pipette pump aspirates and dispenses samples. An air manifold is connected to a pneumatic pump to open and close the normally closed valve. The pneumatic device moves fluid from one position to the next. The pneumatic design has reduced channel cross talk compared to other device designs.

FIG. 55B shows a schematic of a cartridge for use in the quake valve pneumatic device shown in FIG. 55A. The valve configuration is shown. The normally closed valves (one such valve is indicated by an arrow) comprise an elastomeric seal on top of the channel to isolate each chamber from the rest of the system when the chamber is not in use. The pneumatic pump uses air to open and close the valve as needed to move fluid to the necessary chambers within the cartridge.

FIG. 56 shows a valve circuitry layout for the pneumatic valve device shown in FIG. 55A. A sample is placed in the sample well while all valves are closed, as shown at (i.). The sample is lysed in the sample well. The lysed sample is moved from the sample chamber to a second chamber by opening the first quake valve, as shown at (ii.), and aspirating the sample using the pipette pump. The sample is then moved to the first amplification chamber by closing the first quake valve and opening a second quake valve, as shown at (iii.) where it is mixed with the amplification mixture. After the sample is mixed with the amplification mixture, it is moved to a subsequent chamber by closing the second quake valve and opening a third quake valve, as shown at (iv). The sample is moved to the DETECTR chamber by closing the third quake valve and opening a fourth quake valve, as shown at (v). The sample can be moved through a different series of chambers by opening and closing a different series of normally open (e.g., quake type) valves, as shown at (vi). Actuation of individual valves in the desired chamber series prevents cross contamination between channels.

FIG. 57 shows a schematic of a sliding valve device. The offset pitch of the channels allows aspirating and dispensing into each well separately and helps to mitigate cross talk between the amplification chambers and corresponding chambers.

FIG. 58 shows a diagram of sample movement through the sliding valve device shown in FIG. 57. In the initial closed position (i.), the sample is loaded into the sample well and lysed. The sliding valve is then actuated by the instrument, and samples are loaded into each of the channels using the pipette pump, which dispenses the appropriate volume into the channel (ii.). The sample is delivered to the amplification chambers by actuating the sliding valve and mixed with the pipette pump (iii.). Samples from the amplification chamber are aspirated into each channel (iv.) and then dispensed and mixed into each DETECTR chamber (v.) by actuating the sliding valve and pipette pump.

FIG. 59 shows a schematic of the top layer of a cartridge of a pneumatic valve device of the present disclosure, highlighting suitable dimensions. The schematic shows one cartridge that is 2 inches by 1.5 inches.

FIG. 60 shows a schematic of a modified top layer of a cartridge of a pneumatic valve device of the present disclosure adapted for electrochemical dimension. In this schematic, three lines are shown in the detection chambers (4 chambers at the very right). These three lines represent wiring (or “metal leads”), which is co-molded, 3D-printed, or manually assembled in the disposable cartridge to form a three-electrode system.

FIG. 61 shows schemes for designing primers for loop mediated isothermal amplification (LAMP) of a target nucleic acid sequence. Regions denoted by “c” are reverse complementary to the corresponding region not denoted by “c” (e.g., region F3c is reverse complementary to region F3).

FIGS. 62A-62D show schematics of exemplary configurations of various regions of a nucleic acid sequence that correspond to or anneal LAMP primers, or guide RNA sequences, or that comprise protospacer-adjacent motif (PAM) or protospacer flanking site (PFS), and target nucleic acid sequences for amplification and detection by LAMP and DETECTR.

FIG. 62A shows a schematic of an exemplary arrangement of the guide RNA (gRNA) with respect to the various regions of the nucleic acid sequence that correspond to or anneal LAMP primers. In this arrangement, the guide RNA is reverse complementary to a sequence of the target nucleic acid, which is between an F1c region (i.e., a region reverse complementary to an F1 region) and a B1 region.

FIG. 62B shows a schematic of an exemplary arrangement of the guide RNA sequence with respect to the various regions of the nucleic acid sequence that correspond to or anneal LAMP primers. In this arrangement, the guide RNA is partially reverse complementary to a sequence of the target nucleic acid, which is between an F1c region and a B1 region. For example, the target nucleic acid comprises a sequence between an F1c region and a B1 region that is reverse complementary to at least 60% of a guide nucleic acid. In this arrangement, the guide RNA is not reverse complementary to the forward inner primer or the backward inner primer shown in FIG. 40.

FIG. 62C shows a schematic of an exemplary arrangement of the guide RNA with respect to the various regions of the nucleic acid sequence that correspond to or anneal LAMP primers. In this arrangement, the guide RNA hybridizes to a sequence of the target nucleic acid, which is within the loop region between the B1 region and the B2 region. The forward inner primer, backward inner primer, forward outer primer, and backward outer primer sequences do not contain and are not reverse complementary to the PAM or PFS.

FIG. 62D shows a schematic of an exemplary arrangement of the guide RNA with respect to the various regions of the nucleic acid sequence that correspond to or anneal LAMP primers. In this arrangement, the guide RNA hybridizes to a sequence of the target nucleic acid, which is within the loop region between the F2c region and F1c region. The primer sequences do not contain and are not reverse complementary to the PAM or PFS.

FIGS. 63A-63C show schematics of exemplary configurations of various regions of the nucleic acid sequence that correspond to or anneal LAMP primers, or guide RNA sequences, or comprise protospacer-adjacent motif (PAM) or protospacer flanking site (PFS), and target nucleic acid sequences for combined LAMP and DETECTR for amplification and detection, respectively. At the right, the schematics also show corresponding fluorescence data using the LAMP amplification and guide RNA sequences to detect the presence of a target nucleic acid sequence, where a fluorescence signal is the output of the DETECTR reaction and indicates presence of the target nucleic acid.

FIG. 63A shows a schematic of an arrangement of various regions of the nucleic acid sequence that correspond to or anneal LAMP primers and positions of three guide RNAs (gRNA1, gRNA2, and gRNA3) relative to the LAMP primers (at left). gRNA1 overlaps with the B2c region and is, thus, reverse complementary to the B2 region. gRNA2 overlaps with the B1 region and is, thus, reverse complementary to the B1c region. gRNA3 partially overlaps with the B3 region and partially overlaps with the B2 region and is, thus, partially reverse complementary to the B3c region and partially reverse complementary to the B2c region. The complementary regions (B1, B2c, B3c, F1, F2c, and F3c) are not depicted, but correspond to the regions shown in FIG. 40. At right is a graph of fluorescence from the DETECTR reaction in the presence of 10,000 genome copies of the target nucleic acid or 0 genome copies of the target nucleic acid.

FIG. 63B shows a schematic of an arrangement of various regions of nucleic acid sequence that correspond to or anneal LAMP primers and positions of three guide RNAs (gRNA1, gRNA2, and gRNA3) relative to the LAMP primers (at left). gRNA1 overlaps with the B1c region and is, thus, reverse complementary to the B1 region. gRNA2 overlaps with the LF region and is, thus, reverse complementary to the LFc region. gRNA 3 partially overlaps with the B2 region and partially overlaps with the LBc region and is, thus, partially reverse complementary to the B2c region and is partially reverse complementary to the LB region. At right is a graph of fluorescence from the DETECTR reaction in the presence of 10,000 genome copies of the target nucleic acid or 0 genome copies of the target nucleic acid.

FIG. 63C shows a schematic of an arrangement of various regions of the nucleic acid sequence that correspond to or anneal LAMP primers and positions of three guide RNAs (gRNA1, gRNA2, and gRNA3) relative to the LAMP primers (at left). gRNA1 overlaps with the B1c region and is, thus, reverse complementary to the B1 region. gRNA2 partially overlaps with the LF region and partially overlaps with the F2c region and is, thus, partially reverse complementary to the LFc region and partially reverse complementary to the F2 region. gRNA3 overlaps with the B2 and is, thus, reverse complementary to the B2c region. At right is a graph of fluorescence from the DETECTR reaction in the presence of 10,000 genome copies of the target nucleic acid or 0 genome copies of the target nucleic acid.

FIG. 64A shows a detailed breakdown of the arrangement and sequences of various regions of the nucleic acid sequence that correspond to or anneal LAMP primers or guide RNA sequences, or comprise protospacer-adjacent motif (PAM) or protospacer flanking site (PFS), and target nucleic acid sequences for the LAMP and DETECTR assays shown in FIG. 63A. FIG. 64A discloses SEQ ID NO: 393.

FIG. 64B shows a detailed breakdown of the arrangement and sequences of various regions of the nucleic acid sequence that correspond to or anneal LAMP primers or guide RNA sequences, or comprise protospacer-adjacent motif (PAM) or protospacer flanking site (PFS), and target nucleic acid sequences for the LAMP and DETECTR assays shown in FIG. 63B. FIG. 64B discloses SEQ ID NO: 393.

FIG. 64C shows a detailed breakdown of the arrangement and sequences of various regions of the nucleic acid sequence that correspond to or anneal LAMP primers or guide RNA sequences, or comprise protospacer-adjacent motif (PAM) or protospacer flanking site (PFS), and target nucleic acid sequences for the LAMP and DETECTR assays shown in FIG. 63C. FIG. 64C discloses SEQ ID NO: 393.

FIG. 65 shows the time to result of a reverse-transcription LAMP (RT-LAMP) reaction detected using a DNA binding dye.

FIG. 66 shows fluorescence signal from a DETECTR reaction following a five-minute incubation with products from RT-LAMP reactions. LAMP primer sets #1-6 in FIG. 65 were designed for use with guide RNA #2 (SEQ ID NO: 250), and LAMP primer sets #7-10 were designed for use with guide RNA #1 (SEQ ID NO: 249).

FIG. 67 shows detection of sequences from influenza A virus (IAV) using SYTO 9 (a DNA binding dye) following RT-LAMP amplification with LAMP primer sets 1, 2, 4, 5, 6, 7, 8, 9, 10, or a negative control.

FIG. 68 shows the time to amplification of an influenza B virus (IBV) target sequence following RT-LAMP amplification. Amplification was detected using SYTO 9 in the presence of increasing concentrations of target sequence (0, 100, 1000, 10,000, or 100,000 genome copies of the target sequence per reaction).

FIG. 69 shows the time to amplification of an IAV target sequence following LAMP amplification with different primer sets.

FIG. 70 shows detection of target nucleic acid sequences from influenza A virus (IAV) using DETECTR following RT-LAMP amplification with LAMP primer sets 1, 2, 4, 5, 6, 7, 8, 9, 10, or a negative control. Ten reactions were performed per primer set. DETECTR signal was measured as a function of an amount of target sequence present in the reaction.

FIG. 71 shows a scheme for designing primers for LAMP amplification of a target nucleic acid sequence and detection of a single nucleotide polymorphism (SNP) in the target nucleic acid sequence. In an exemplary arrangement, the SNP of the target nucleic acid is positioned between the F1c region and the B1 region.

FIGS. 72A-72C show schematics of exemplary arrangements of LAMP primers, guide RNA sequences, protospacer-adjacent motif (PAM) or protospacer flanking site (PFS), and target nucleic acids with a SNP for methods of LAMP amplification of a target nucleic acid and detection of the target nucleic acid using DETECTR.

FIG. 72A shows a schematic of an exemplary arrangement of the guide RNA with respect to various regions of the nucleic acid sequence that correspond to or anneal LAMP primers. In this arrangement, the PAM or PFS of the target nucleic acid is positioned between an F1c region and a B1 region. The entirety of the guide RNA sequence may be between the F1c region and the B1c region. The SNP is shown as positioned within a sequence of the target nucleic acid that hybridizes to the guide RNA.

FIG. 72B shows a schematic of an exemplary arrangement of the guide RNA sequence with respect to various regions of the nucleic acid sequence that correspond to or anneal LAMP primers. In this arrangement, the PAM or PFS of the target nucleic acid is positioned between an F1c region and a B1 region and the target nucleic acid comprises a sequence between an F1c region and a B1 region that is reverse complementary to at least 60% of a guide nucleic acid. In this example, the guide RNA is not reverse complementary to the forward inner primer or the backward inner primer. The SNP is shown as positioned within a sequence of the target nucleic acid that hybridizes to the guide RNA.

FIG. 72C shows a schematic of an exemplary arrangement of the guide RNA sequence with respect to various regions of the nucleic acid sequence that correspond to or anneal LAMP primers. In this arrangement, the PAM or PFS of the target nucleic acid is positioned between the F1c region and the B1 region and the entirety of the guide RNA sequence is between the F1c region and the B1 region. The SNP is shown as positioned within a sequence of the target nucleic acid that hybridizes to the guide RNA.

FIG. 73 shows an exemplary sequence (SEQ ID NO: 394) of a nucleic acid comprising two PAM sites and a HERC2 SNP.

FIG. 74 shows results from DETECTR reactions to detect a HERC2 SNP at position 9 with respect to a first PAM site or position 14 with respect to a second PAM site following LAMP amplification. Fluorescence signal, indicative of detection of the target sequence, was measured over time in the presence of a target sequence comprising either a G allele or an A allele in HERC2. The target sequence was detected using a guide RNA (crRNA only) to detect either the A allele or the G allele.

FIG. 75 shows a heatmap of fluorescence from a DETECTR reaction following LAMP amplification of the target nucleic acid sequence. The DETECTR reaction differentiated between two HERC2 alleles, using guide RNAs (crRNA only) specific for the A allele (SEQ ID NO: 255, “R570 A SNP”) or the G SNP allele (SEQ ID NO: 256, “R571 G SNP”). Positive detection is indicated by a high fluorescence value in the DETECTR reaction.

FIG. 76 shows combined LAMP amplification of a target nucleic acid by LAMP and detection of the target nucleic acid by DETECTR. Detection was carried out visually with DETECTR by illuminating the samples with a red LED. Each reaction contained a target nucleic acid sequence comprising a SNP allele for either a blue eye phenotype (“Blue Eye”) or a brown eye phenotype (“Brown Eye”). Samples “Brown *” and “Blue *” were an A allele positive control and a G allele positive control, respectively. A guide RNA for either the brown eye phenotype (“Br”) or the blue eye phenotype (“Bl”) was used for each LAMP DETECTR reaction.

FIG. 77 illustrates schematically the steps of preparing and detecting the presence or absence of SARS-CoV-2 (“2019-nCoV”) in a sample using reverse transcription and loop-mediated isothermal amplification (RT-LAMP) and Cas12 illustrates schematically the steps of preparing and detecting the presence or absence of SARS-CoV-2 (“2019-nCoVreactions.

FIG. 78 shows the DETECTR assay results of the SARS-CoV-2 N-gene amplified with different primer sets (“2019-nCoV-set1” through “2019-nCoV-set12”) and detected using LbCas12a and a gRNA directed to the N-gene of SARS-CoV-2. A lower time to result is indicative of a positive result. For all primer sets, the time to result was lower for samples with more of the target sequence, indicating that the assay was sensitive for the target sequence.

FIG. 79 shows the individual traces of the DETECTR reactions plotted in FIG. 78 for the 0 fM and 5 fM samples. In each plot, the 0 fM trace is not visible above the baseline, indicating that there little to no non-specific detection.

FIG. 80 shows the time to result of a DETECTR reaction on samples containing either the N-gene, the E-gene, or no target (“NTC”) and amplified using primer sets directed to the E-gene of SARS-CoV-2 (“2019-nCoV-E-set13” through “2019-nCoV-E-set20”) or to the N-gene of SARS-CoV-2 (“2019-nCoV-N-set21” through “2019-nCoV-N-set24”). The best performing primer set for specific detection of the SARS-CoV-2 E-gene was SARS-CoV-2-E-set14.

FIG. 81 shows the DETECTR assay results of the SARS-CoV-2 N-gene amplified with primer set 1 (“2019-nCoV-set1”) and detected using LbCas12a and either a gRNA directed to the N-gene of SARS-CoV-2 (“R1763—CDC-N2-Wuhan”) or a gRNA directed to the N-gene of SARS-CoV (“R1766—CDC-N2-SARS”).

FIG. 82 shows the results of a DETECTR reaction to determine the limit of detection of SARS-CoV-2 in a DETECTR reaction amplified using a primer set directed to the N-gene of SARS-CoV-2 (“2019-nCoV-N-set1”). Samples containing either 15,000, 4,000, 1,000, 500, 200, 100, 50, 20, or 0 copies of a SARS-CoV-2 N-gene target nucleic acid were detected. A gel of the N-gene RNA is shown below.

FIG. 83 shows the amplification of RNase P using a POP7 sample primer set. Samples were amplified using LAMP. DETECTR reactions were performed using a gRNA directed to RNase P (“R779”) and a Cas12 variant (SEQ ID NO: 37). Samples contained either HeLa total RNA or HeLa genomic DNA.

FIG. 84 shows the time to result of a multiplexed DETECTR reaction. Samples contained either in vitro transcribed N-gene of SARS-CoV-2 (“N-gene IVT”), in vitro transcribed E-gene of SARS-CoV-2 (“E-gene IVT”), HeLa total RNA, or no target (“NTC”). Samples were amplified using one or more primer sets directed to the SARS-CoV-2 N-gene (“set1”), the SARS-CoV-2 E-gene (“set14”), or RNase” (“RNaseP”).

FIG. 85 shows the time to results of a multiplexed DETECTR reaction with different combinations of primer sets directed to either SARS-CoV-2 N-gene (“set1”), SARS-CoV-2 E-gene (“set14”), or RNase P (“RNaseP”). Samples containing in vitro transcribed N-gene of SARS-CoV-2 (left, “N-gene IVT”) or in vitro transcribed E-gene of SARS-CoV-2 (right, “E-gene IVT”) were tested.

FIG. 86 shows the time to result of a multiplexed DETECTR reaction with the best performing primer set combinations from FIG. 84 and FIG. 85.

FIG. 87A schematically illustrates the sequence of the CDC-N2 target site used for detecting the N-2 gene of SARS-CoV-2. FIG. 87A discloses SEQ ID NOS 395-398, respectively, in order of appearance.

FIG. 87B schematically illustrates the sequence of a region of the SARS-CoV-2 N-gene (“N-Sarbeco”) target site. FIG. 87B discloses SEQ ID NOS 399-400 and 400-401, respectively, in order of appearance.

FIG. 88 shows the results of a DETECTR assay to determine the sensitivity of gRNAs directed to either N-gene of SARS-CoV-2 (“R1763”), the N-gene of SARS-CoV (“R1766”), or the N-gene of a Sarbeco coronavirus (“R1767”) for samples containing either the N-gene of SARS-CoV-2(“N-2019-nCoV”), the N-gene of SARS-CoV (“N-SARS-CoV”), or the N-gene of bat-SL-CoV45 (“N-bat-SL-CoV45”).

FIG. 89 schematically illustrates the sequence of a region of the SARS-CoV-2 E-gene (“E-Sarbeco”) target site. FIG. 89 discloses SEQ ID NOS 402-403 and 403-404, respectively, in order of appearance.

FIG. 90 shows the results of a DETECTR assay to determine the sensitivity of two gRNAs directed to a coronavirus N-gene for samples containing either the E-gene of SARS-CoV-2 (“E-2019-nCoV”), the E-gene of SARS-CoV (“E-SARS-CoV”), the E-gene of bat-SL-CoV45 (“E-bat-SL-CoV45”), or the E-gene of bat-SL-CoV21 (“E-bat-SL-CoV21”).

FIG. 91 shows the results of a lateral flow DETECTR reaction to detect the presence or absence of a SARS-CoV-2 N-gene target RNA using a Cas12 variant (SEQ ID NO: 37). Lateral flow test strips are shown. Samples either containing (“+”) or lacking (“−”) in vitro transcribed SARS-CoV-2 N-gene RNA (“N-gene IVT”) were tested. The top set of horizontal lines (denoted “test”) indicated the results of the DETECTR reaction.

FIG. 92 illustrates schematically the detection of a target nucleic acid using a programmable nuclease. Briefly, a Cas protein with trans collateral cleavage activity is activated upon binding to a guide nucleic acid and a target sequence reverse complementary to a region of the guide nucleic acid. The activated programmable nuclease cleaves a reporter nucleic acid, thereby producing a detectable signal.

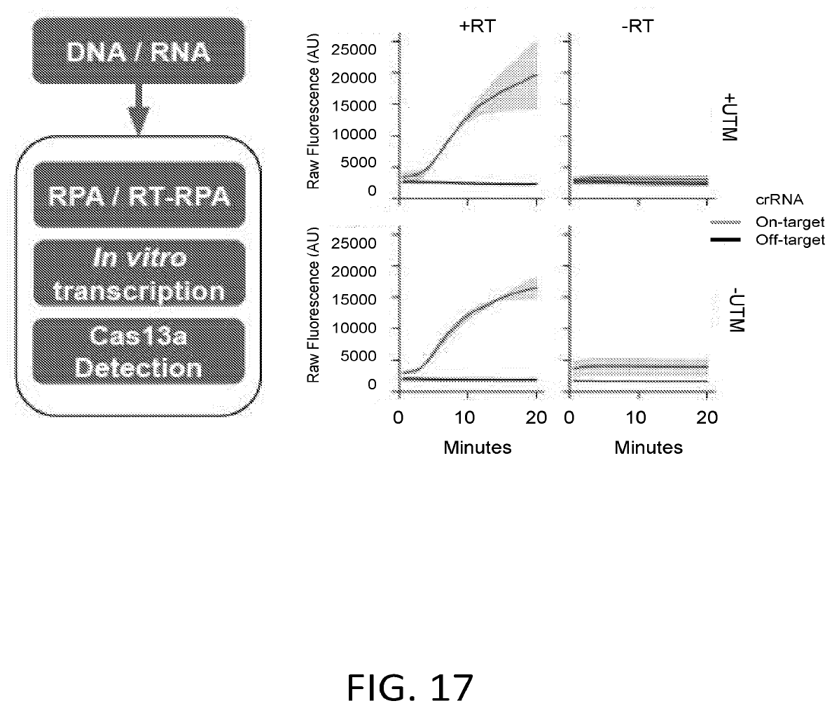

FIG. 93 illustrates schematically detection of the presence or absence of a target nucleic acid in a sample. Select nucleic acids in a sample are amplified using isothermal amplification. The amplified sample is contacted to a programmable nuclease, a guide nucleic acid, and a reporter nucleic acid, as illustrated in FIG. 17. If the sample contains the target nucleic acid, a detectable signal is produced.

FIG. 94 shows the results of a DETECTR lateral flow reaction to detect the presence or absence of SARS-CoV-2 (“2019-nCoV”) RNA in a sample. Detection of RNase P is used as a sample quality control. Samples were in vitro transcribed and amplified (left) and detected using a Cas12 programmable nuclease (right). Samples containing (“+”) or lacking (“−”) in vitro transcribed SARS-CoV-2 RNA (“2019-nCoV IVT”) were assayed with a Cas12 programmable nuclease and gRNA directed to SARS-CoV-2 for either 0 min or 5 min. The reaction was sensitive for samples containing SARS-CoV-2.

FIG. 95 shows the results of a DETECTR reaction using an LbCas12a programmable nuclease (SEQ ID NO: 27) to determine the presence or absence of SARS-CoV-2 in patient samples.

FIG. 96 shows the results of a lateral flow DETECTR reaction to detect the presence or absence of SARS-CoV-2 in patient samples. Samples were detected with either a gRNA directed to SARS-CoV-2 or a gRNA directed to RNase P.

FIG. 97 shows technical specifications and assay conditions for detection of coronavirus using reverse transcription and loop-mediated isothermal amplification (RT-LAMP) and Cas12 detection.

FIG. 98 shows the results of a DETECTR assay evaluating multiple gRNAs for detecting SARS-CoV-2 using LbCas12a. Target nucleic acid sequences were amplified using primer sets to amplify the SARS-CoV-2 E-gene (“2019-nCoV-E-set13” through “2019-nCoV-E-set20” or the SARS-CoV-2 N-gene (“2019-nCoV-N-set21” through “2019-nCoV-N-set24”).

FIG. 99 shows the results of a DETECTR assay evaluating multiple gRNAs for their utility in distinguishing between three different strains of coronavirus, SARS-CoV-2 (“COVID-2019”), SARS-CoV, or bat-SL-CoV45. Samples containing N-gene amplicons of either SARS-CoV-2 (“N-2019-nCoV”), SARS-CoV (“N-SARS-CoV”), or bat-SL-CoV45 (“N-bat-SL-CoV45”) were tested.

FIG. 100 shows the results of a DETECTR assay evaluating multiple gRNAs for their utility in distinguishing between three different strains of coronavirus, SARS-CoV-2 (“COVID-2019”), SARS-CoV, or bat-SL-CoV45. Samples containing E-gene amplicons of either SARS-CoV-2 (“N-2019-nCoV”), SARS-CoV (“N-SARS-CoV”), or bat-SL-CoV45 (“N-bat-SL-CoV45”) were tested.

FIG. 101 shows the results of a DETECTR assay evaluating LAMP primer sets for their utility in multiplexed amplification of SARS-CoV-2 targets. Samples were amplified with one or more primer sets directed to the SARS-CoV-2 N-gene (“set1”) or the SARS-CoV-2 E-gene (“set14”), or RNase P (“RNaseP”).

FIG. 102 shows the results of a DETECTR assay evaluating the sensitivity of an RT-LAMP amplification reaction to common sample buffers. Reactions were measured in universal transport medium (UTM, top) or DNA/RNA Shield buffer (bottom) at different buffer dilutions (from left to right: 1×, 0.5×, 0.25×, 0.125×, or no buffer).

FIG. 103 shows the results of a DETECTR assay to determine the limit of detection (LoD) of the DETECTR assay for SARS-CoV-2 (the virus attributed to the COVID-19 infection).

FIG. 104 shows the results of a DETECTR assay evaluating the target specificity of a gRNA directed to the N-gene of SARS-CoV-2 (“R1763-N-gene”) in a 2-plex multiplexed RT-LAMP reaction using an LbCas12a programmable nuclease (SEQ ID NO: 27).

FIG. 105 shows the results of a DETECTR assay evaluating the target specificity of a gRNA directed to the N-gene of SARS-CoV-2 (“R1763-N-gene”) or the E-gene of SARS-CoV-2 (“R1765-E-gene”) in a 3-plex multiplexed RT-LAMP reaction using an LbCas12a programmable nuclease (SEQ ID NO: 27).

FIG. 106 illustrates the design of detector nucleic acids compatible with a PCRD lateral flow device. Exemplary compatible detector nucleic acids, rep072, rep076, and rep100, are provided (left). These detector nucleic acids may be used in a PCRD lateral flow device (right) to detect the presence or absence of a target nucleic acid. The top right schematic illustrates an exemplary band configuration produced when contacted to a sample that does not contain a target nucleic acid. The bottom right schematic shows an exemplary band configuration produced when contacted to a sample that does contain a target nucleic acid. FIG. 106 discloses SEQ ID NOS 372 and 372, respectively, in order of appearance.

FIG. 107A illustrates a genome map indicating the locations of the E (envelope) gene and the N (nucleoprotein) gene regions within a coronavirus genome. Corresponding regions or annealing regions of primers and probes relative to the E and N gene regions are shown below the respective gene regions. RT-LAMP primers are indicated by black rectangles, the binding position of the F1c and B1c half of the FIP primer (grey) is represented by a striped rectangle with dashed borders. Regions amplified in tests utilized by the World Health Organization (WHO) and the Center for Disease Control (CDC) are denoted as “WHO E amplicon” and “CDC N2 amplicon,” respectively.

FIG. 107B shows the results of a DETECTR assay evaluating the specificity or broad detection utility of gRNAs directed to the N-gene or E-gene of various coronavirus strains (SARS-CoV-2, SARS-CoV, or bat-SL-CoVZC45) using an LbCas12a programmable nuclease (SEQ ID NO: 27). The N gene gRNA used in the assay (left, “N-gene”) was specific for SARS-CoV-2, whereas the E gene gRNA was able to detect 3 SARS-like coronavirus (right, “E-gene”). A separate N gene gRNA targeting SARS-CoV and a bat coronavirus failed to detect SARS-CoV-2 (middle, “N-gene related species variant”).

FIG. 107C shows exemplary laboratory equipment utilized in the coronavirus DETECTR assays. In addition to appropriate biosafety protective equipment, the equipment utilized includes a sample collection device, microcentrifuge tubes, heat blocks set to 37° C. and 62° C., pipettes and tips, and lateral flow strips.

FIG. 107D illustrates an exemplary workflow of a DETECTR assay for the detection of a coronavirus in a subject. Conventional RNA extraction or sample matrix can be used as an input to DETECTR (LAMP pre-amplification and Cas12-based detection for NE gene, EN gene and RNase P), which is visualized by a fluorescent reader or lateral flow strip.

FIG. 107E shows lateral flow test strips (left) indicating a positive test result for SARS-CoV-2 N-gene (left, top) and a negative test result for SARS-CoV-2 N-gene (left, bottom). The table (right) illustrates possible test indicators and associated results for a lateral flow strip-based coronavirus diagnostic assay that tests for the presences of absence of the RNase P (positive control), SARS-CoV-2 N-gene, and coronavirus E-gene.

FIG. 108A illustrates cleavage of a detector nucleic acid labeled with FAM and biotin by a Cas12 programmable nuclease in the presence of a target nucleic acid (top). Schematics of lateral flow test strips (bottom) illustrate markings indicative of either the presence (“positive”) or absence (“negative”) of the target nucleic acid in the tested sample. The intact FAM-biotinylated reporter molecule flows to the control capture line. Upon recognition of the matching target, the Cas-gRNA complex cleaves the reporter molecule, which flows to the target capture line.

FIG. 108B shows the results of a DETECTR assay using LbCas12a to determine the effect of reaction time for a sample containing either 0 fM SARS-CoV-2 RNA or 5 fM SARS-CoV-2 RNA. Fluorescence signal of LbCas12a detection assay on RT-LAMP amplicon for SARS-CoV-2 N-gene saturated within 10 minutes. RT-LAMP amplicon was generated from 2 μLof 5 fM or 0 fM SARS-CoV-2 N-gene IVT RNA by amplifying at 62° C. for 20 minutes.

FIG. 108C shows lateral flow test strips assaying samples corresponding to the samples assayed by DETECTR in FIG. 108B. Bands corresponding to control (C) or test (T) are shown for samples containing either 0 fM SARS-CoV-2 RNA (“−”) or 5 fM SARS-CoV-2 RNA (“+”) as a function of reaction time. LbCas12a on the same RT-LAMP amplicon produced visible signal through lateral flow assay within 5 minutes.

FIG. 108D shows the results of a DETECTR assay with LbCas12a (middle) or a CDC protocol (left) to determine the limit of detection of SARS-CoV-2. Signal is shown as a function of the number of copies of viral genome per reaction. Representative lateral flow results for the assay shown for 0 copies/μL and 10 copies/μL (right).

FIG. 108E shows patient sample DETECTR data. Clinical samples from 6 patients with COVID-19 infection (n=11, 5 replicates) and 12 patients infected with influenza or one of the 4 seasonal coronaviruses (HCoV-229E, HCoV-HKU1, HCoV-NL63, HCoV-OC43) (n=12) were analyzed using SARS-CoV-2 DETECTR (shaded boxes). Signal intensities from lateral flow strips were quantified using ImageJ and normalized to the highest value within the N gene, E gene or RNase P set, with a positive threshold at five standard deviations above background. Final determination of the SARS-CoV-2 test was based on the interpretation matrix in FIG. 107E. FluA denotes Influenza A, and FluB denotes Influenza B. HCoV denotes human coronavirus.

FIG. 108F shows lateral flow test strips testing for SARS-CoV-2 in a patient with COVID-19 (positive for SARS-CoV-2, “patient 1”), a no target control sample lacking the target nucleic acid (“NTC”), and a positive control sample containing the target nucleic acid (“PC”). All three samples were tested for the presence of the SARS-CoV-2 N-gene, the SARS-CoV-2 E-gene, and RNase P.

FIG. 108G shows performance characteristics of the SARS-CoV-2 DETECTR assay. 83 clinical samples (41 COVID-19 positive, 42 negative) were evaluated using the fluorescent version of the SARS-CoV-2 DETECTR assay. One sample (COVID19-3) was omitted due to failing assay quality control. Positive and negative calls were based on criteria described in FIG. 32E. fM denotes femtomolar; NTC denotes no-template control; PPA denotes positive predictive agreement; NPA denotes negative predictive agreement.

FIG. 109 shows a table comparing the SARS-CoV-2 DETECTR assay with RT-LAMP of the present disclosure to the SARS-CoV-2 assay with a quantitative reverse transcription polymerase chain reaction (qRT-PCR) detection method. The N-gene target in the DETECTR RT-LAMP assay is the same as the N-gene N2 amplicon detected in the qRT-PCR assay.

FIG. 110A shows the time to result of an RT-LAMP amplification under different buffer conditions. Time to results was calculated as the time at which the fluorescent value is one third of the max for the experiment. Reactions that failed to amplify are reported with a value of 20 minutes and labeled as “no amp.” Time to result was determined for different starting concentrations of target control plasmid in either water, 10% phosphate buffered saline (PBS), or 10% universal transport medium (UTM). A lower time to result indicates faster amplification.

FIG. 110B shows the results of an RT-LAMP assay to determine the amplification efficiency of the N-gene of SARS-CoV-2, the E-gene of SARS-CoV-2, and RNase P in either 5% UTM, 5% PBS, or water. Samples containing 0.5 fM N-gene in vitro transcribed, 0.5 fM of E-gene in vitro transcribed, and 0.8 ng/μL HeLa total RNA (“N+E+total RNA”) or no target controls (“NTC”) were tested.

FIG. 110C shows amplification of RNA directly from nasal swabs in PBS. Time to result was measured as a function of PBS concentration. Nasal swabs (“nasal swab”) were either spiked with HeLa total RNA (left, “total RNA: 0.08 ng/uL”) or water (right, “total RNA: 0 ng/uL”). Samples without a nasal swab (“no swab”) were compared as controls.

FIG. 111A shows raw fluorescence curves generated by LbCas12a (SEQ ID NO: 27) detection of SARS-CoV-2 N-gene (n=6). The curves showed saturation in less than 20 minutes.

FIG. 111B shows the limit of detection of a DETECTR assay for the SARS-CoV-2 N-gene detected with LbCas12a, as determined from the raw fluorescence traces shown in FIG. 111A. Fluorescence intensity was measured with decreasing concentration (copies per mL) of SARS-CoV-2 N-gene.

FIG. 111C shows the time to result of the limit of detection DETECTR assay, as determined from the raw fluorescence traces shown in FIG. 111A. A lower time to result indicates faster amplification and detection.

FIG. 112A shows the results of a DETECTR assay using LbCas12a to determine the effect of reaction time for a sample containing either 0 fM SARS-CoV-2 RNA or 5 fM SARS-CoV-2 RNA.

FIG. 112B shows lateral flow test strips assaying samples corresponding to the samples assayed by DETECTR in FIG. 112A. Bands corresponding to control (C) or test (T) are shown for samples containing either 0 fM SARS-CoV-2 RNA (“−”) or 5 fM SARS-CoV-2 RNA (“+”) as a function of reaction time.

FIG. 113 shows the results of a DETECTR assay to determine the cross-reactivity of gRNAs for different human coronavirus strains. Samples containing in vitro transcribed RNA of the SARS-CoV-2 N-gene, the SARS-CoV N-gene, the bat-SL-CoVZC45 N-gene, the SARS-CoV-2 E-gene, the SARS-CoV E-gene, or the bat-SL-CoVZC45 E-gene, or clinical samples positive for CoV-HKU1, CoV-299E, CoV-OC43, or CoV-NL63 were tested. HeLa total RNA was tested as a positive control for RNase P, and a sample lacking a target nucleic acid (“NTC”) was tested as a negative control.

FIG. 114A shows a sequence alignment (SEQ ID NOS: 405-410, respectively, in order of appearance)of the target sites targeted by the N-gene gRNA for three coronavirus strains. The N gene gRNA #1 is compatible with the CDC-N2 amplicon, the N gene gRNA #2 is compatible with WHO N-Sarbeco amplicon.

FIG. 114B shows a sequence alignment (SEQ ID NOS: 411-416, respectively, in order of appearance) of the target sites targeted by the E-gene gRNA for three coronavirus strains. The two E gene gRNAs tested (E gene gRNA #1 and E gene gRNA #2) are compatible with the WHO E-Sarbeco amplicon.

FIG. 115A-FIG. 115C show DETECTR kinetic curves on COVID-19 infected patient samples. Ten nasal swab samples from 5 patients (COVID19-1 to COVID19-10) were tested for SARS-CoV-2 using two different genes, N2 and E as well as a sample input control, RNase P. FIG. 115A shows using the standard amplification and detection conditions, 9 of the 10 patients resulted in robust fluorescence curves indicating presence of the SARS-CoV-2 E-gene (20 minute amplification, signal within 10 minutes). FIG. 115B shows the SARS-CoV-2 N-gene required extended amplification time to produce strong fluorescence curves (30 minute amplification, signal within 10 minutes) for 8 of the 10 patients. FIG. 115C shows that as a sample input control, RNase P was positive for 17 of the 22 total samples tested (20 minute amplification, signal within 10 minutes).

FIG. 116 shows DETECTR analysis of SARS-CoV-2 identifies down to 10 viral genomes in approximately 30 min (20 min amplification, 10 min DETECTR). Duplicate LAMP reactions were amplified for twenty min followed by LbCas12a DETECTR analysis.

FIG. 117 shows the raw fluorescence at 5 minutes for the LbCas12a DETECTR analysis provided in FIG. 116. The limit of detection of the SARS-CoV-2 N-gene was determined to be 10 viral genomes per reaction (n=6).

FIG. 118 shows lateral flow DETECTR results on 10 COVID-19 infected patient samples and 12 patient samples for other viral respiratory infections. Ten samples from 6 patients (COVID19-1 to COVID19-5) with one nasopharyngeal swab (A) and one oropharyngeal swab (B) were tested for SARS-CoV-2 using two different genes, N2 and E as well as a sample input control, RNase P. Results were analyzed in accordance with the guidance provided in FIG. 119.

FIG. 119 shows instructions for the interpretation of SARS-CoV-2 DETECTR lateral flow results.

FIG. 120A-C show fluorescent DETECTR kinetic curves performed on 11 COVID-19 infected patient samples and 12 patient samples for other viral respiratory infections. Ten nasopharyngeal/oropharyngeal swab samples from 6 patients (COVID19-1 to COVID19-6) were tested for SARS-CoV-2 using two different genes, N2 and E as well as a sample input control, RNase P.

FIG. 120A shows samples tested using the standard amplification and detection conditions, 10 of the 12 COVID-19 positive patient samples resulted in robust fluorescence curves indicating presence of the SARS-CoV-2 E gene (20-minute amplification, signal within 10 min). No E gene signal was detected in the 12 other viral respiratory clinical samples.

FIG. 120B shows samples tested for the presence of the SARS-CoV-2 N gene using an extended amplification time to produce strong fluorescence curves (30-minute amplification, signal within 10 min) for 10 of the 12 COVID-19 positive patient samples. No N gene signal was detected in the 12 other viral respiratory clinical samples.

FIG. 120C shows graphs corresponding to the sample input control, RNase P.

FIG. 121 shows heatmaps of SARS-CoV-2 DETECTR assay results for clinical samples with the test interpretation indicated. Results of lateral flow SARS-CoV-2 DETECTR assay (top) quantified by ImageJ Gel Analyzer tools for SARS-CoV-2 DETECTR on 24 clinical samples (12 COVID-19 positive) show 98.6% (71/72 strips) agreement with the results of the fluorescent version of the assay (bottom). Both assays were run with 30-minute amplification, Cas12 reaction signal taken at 10 min. Presumptive positive indicated by (+) in orange (bottom, column 4).

FIG. 122 shows heatmaps of SARS-CoV-2 DETECTR assay results for clinical samples with the test interpretation indicated. The top plot shows result of fluorescent SARS-CoV-2 DETECTR assay on an additional 30 COVID-19 positive clinical samples (27 positive, 1 presumptive positive, 2 negative). Presumptive positive indicated by (+) in orange (top, column 9). The bottom plot shows result of fluorescent SARS-CoV-2 DETECTR assay on an additional 30 COVID-19 negative clinical samples (0 positive, 30 negative).

FIG. 123 shows the time to result for RT-LAMP amplification of RNase P POP7 with different primer sets. Time to result was determined for samples amplified with primer sets 1-10. Primer set 1 corresponds to SEQ ID NO: 360-SEQ ID NO: 365, and primer set 9 corresponds to SEQ ID NO: 366-SEQ ID NO: 371.

FIG. 124 shows raw fluorescence over time of a DETECTR reaction performed on RNase P POP7 amplified using RT-LAMP with primer set 1 or primer set 9 and detected with R779, R780, or R1965 gRNAs. The DETECTR reaction was carried out at 37° C. for 90 minutes. The amplicon generated by the set 1 primers were detected without background (dotted line) by R779.

FIG. 125A shows the time to result of RNase P POP7 detection in samples containing 10-fold dilutions of total RNA amplified using RT-LAMP with primer set 1 or primer set 9. Amplification was carried out at 60° C. for 30 minutes.

FIG. 125B shows a DETECTR reaction of the RNase P POP7 amplicons shown in FIG. 125A and detected using gRNA 779 (SEQ ID NO: 330) or gRNA 1965 (SEQ ID NO: 331). Samples amplified using primer set 1 were detected with gRNA 779 and samples amplified with primer set 9 were detected with gRNA 1965. The DETECTR reaction was carried out at 37° C. for 90 minutes.

FIG. 126A and FIG. 126B show photos of cartridges designed for use in a DETECTR assay.

FIG. 127A and FIG. 127B schematic view of the cartridge pictured in FIG. 126A.

FIG. 128A-FIG. 128D show schematics of cartridges designed for use in a DETECTR assay. FIG. 128A shows a cartridge with circular reagent storage wells and a z-direction high resistance serpentine path. FIG. 128B shows a cartridge with elongated reagent storage wells and a z-direction high resistance serpentine path. FIG. 128C shows a cartridge with circular reagent storage wells and an xy-direction high resistance serpentine path. FIG. 128D shows a cartridge with elongated reagent storage wells and an xy-direction high resistance serpentine path.

FIG. 129A-FIG. 129D show schematics of cartridges designed for use in a DETECTR assay. FIG. 129A shows a cartridge with serpentine resistance channels for sample metering which are serpentine on a different plane or layer than the sample metering channel. FIG. 129B shows a cartridge with serpentine resistance channels for sample metering which are serpentine on the same plane or layer than the sample metering channel. FIG. 129C shows a cartridge with right angle arduous path resistance paths for sample metering and a DETECTR sample metering inlet on a different plane or layer than the sample metering channel. FIG. 129D shows a cartridge with right angle arduous path resistance paths for sample metering and a DETECTR sample metering inlet on the same plane or layer than the sample metering channel.

FIG. 130A shows features of a cartridge designed for use in a DETECTR assay.

FIG. 130B shows a manufacturing scheme (left and middle) for manufacturing a cartridge of the present disclosure and a readout device (right) for detecting a sample in a cartridge.

FIG. 131A shows a schematic of a cartridge manifold for heating regions of a cartridge of the present disclosure. The cartridge manifold has an integrated heating zone with integrated air supply connections and integrated O-ring grooves for air supply interface. The cartridge manifold contains an insulation zone to thermally separate the amplification temperature zone from the detection temperature zone and to maintain the appropriate temperature of the amplification chambers and the detection chambers of the cartridge.

FIG. 131B shows two production methods for producing the cartridges described herein. In a first manufacturing method (left), a cartridge is manufactured using two-dimensional (2D) lamination of multiple layers. In a second manufacturing method (right), a part containing consolidated, complex features is injection molded and sealed by lamination.

FIG. 131C shows a schematic of a cartridge with a luer slip adapter for coupling the cartridge to a syringe. The adapter can form a tight fit seal with a slip luer tip. The adapter is configured to function with any of the cartridges disclosed herein.

FIG. 132A and FIG. 132B show schematics of an integrated flow cell for use with a microfluidic cartridge. The integrated flow cell contains three regions, a lysis region, an amplification region, and a detection region. The lysis region is long enough to accommodate a microfluidic chip shop sample lysis flow cell. The lysis flow cell may be combined with the amplification and detection chambers on the cartridges disclosed herein.

FIG. 133 shows details of the inlet channels on a cartridge of the present disclosure.

FIG. 134 shows a workflow for performing a DETECTR assay using a microfluidic cartridge of the present disclosure. The cartridge (“chip”) is loaded with a sample and reaction solutions. The amplification chamber (“LAMP chamber”) is heated to 60° C. and the sample is incubated in the amplification chamber for 30 minutes. The amplified sample (“LAMP amplicon”) is pumped to the DETECTR reaction chambers, and the DETECTR reagents are pumped to the DETECTR reaction chambers. The DETECTR reaction chambers are heated to 37° C. and the sample is incubated for 30 minutes. The fluorescence in the DETECTR reaction chambers is measured in real time to produce a quantitative result.

FIG. 135 shows a schematic of a system electronics architecture of a cartridge manifold compatible with the cartridges disclosed herein. The electronics are configured to heat a first zone of a cartridge to 37° C. and a second zone of the cartridge to 60° C.

FIG. 136A and FIG. 136B show schematics of a cartridge manifold for heating and detecting a cartridge of the present disclosure. The manifold is configured to accept a cartridge, facilitate a DETECTR reaction, and read the resulting fluorescence of the DETECTR reaction.

FIG. 137A shows an example of a fluorescent sample in a cartridge and illuminated with a cartridge manifold. The positive control well contains reagents and an amplified sample following a 30 minute amplification step at 60° C. and a 30 minute detection step at 37° C. The empty well serves as a pseudo negative sample.

FIG. 137B shows a cartridge manifold for heating and detecting a cartridge of the present disclosure.

FIG. 137C shows a cartridge manifold for heating and detecting a cartridge of the present disclosure.

FIG. 138A and FIG. 138B show fluorescence produced in detection chambers of microfluidic cartridges facilitated by manifolds of the present disclosure.

FIG. 139A, FIG. 139B, FIG. 140A, and FIG. 140B show thermal testing summaries for an amplification chamber heated to 60° C. (FIG. 139A and FIG. 140A) or a DETECTR chamber heated to 37° C. (FIG. 139B and FIG. 140B).

FIG. 141A shows the DETECTR results run on a plate reader at a gain of 100, using the LAMP product from the microfluidic cartridge as an input. The samples were run in duplicate with a single non-template control (NTC).

FIG. 141B shows three LAMP products run on a plate reader using samples from a microfluidic chip. The LAMP reactions are numbered in the order that the chips were run (LAMP_1 was run first, etc.). The donor was homozygous for SNP A, and in accordance with that crRNA 570 comes up first. The ATTO 488 was used as a fluorescence standard.

FIG. 142A shows an image of a loaded microfluidic chip.

FIG. 142B shows results of a DETECTR reaction measured on a plate reader after 30 minutes of LAMP amplification.

FIG. 143A, FIG. 143B, FIG. 143C, and FIG. 143D show results of the coronavirus DETECTR reaction. The two reaction chambers with 10 copies input to LAMP resulted in a rapidly increasing DETECTR signal. All NTCs were negative. With 10 copies input into LAMP, the DETECTR signal gradually increased over the course of the reaction, as shown in the photodiode measurements below in FIG. 143C. The negative controls in FIG. 143D indicated an absence of contamination.

FIG. 144A, FIG. 144B, FIG. 144C, and FIG. 144D show the results of the repeated coronavirus DETECTR reaction.

FIG. 145A, FIG. 145B, FIG. 146A, FIG. 146B, and FIG. 146C show the photodiode measurements for an influenza B DETECTR reaction in a microfluidic cartridge.

FIG. 147 shows fluorescence results from a series of DETECTR reagents which had been stored in glass capillaries for 7 months.

FIG. 148 provides a design for a spin-through column and a method for using the spin-through column for sequential amplification and DETECTR reactions.

FIG. 149 provides structures for three reagents used to construct electrochemically detectable nucleic acids: (A) ferrocene-tagged thymidine, (B) 6-carboxyfluorescein, and (C) biotin-tagged phosphate.

FIG. 150 provides a design for an injection molded-cartridge containing a sample input chamber and multiple chambers in which portions of the sample can be subjected to amplification and detector reactions.

FIG. 151 provides a design for a device comprising a detector diode array and heating panels that is capable of utilizing the injection-molded cartridge shown in FIG. 150.

FIG. 152 and FIG. 153 show fluorescence data from a series of DETECTR reactions performed on samples subjected to different dual-lysis amplification buffers.

FIG. 154 panel (a) provides a design for an injection-molded cartridge for performing multiple amplification and DETECTR reactions on a sample. Panel (b) provides a design for a device configured to utilize the injection-molded cartridge and measure fluorescence from the DETECTR reactions performed in the cartridge.

FIG. 155 provides a method for utilizing the injection-molded cartridge and device shown in FIG. 154 for performing parallel amplification and DETECTR reactions on a sample.

FIG. 156 shows diode arrays and dye-loaded reaction compartments from the injection-molded cartridge and device in FIG. 154.

FIG. 157 shows a possible design for an injection molded cartridge comprising one sample chamber connected to 5 amplification chamber, and 2 Detection chambers connected to each amplification chamber. Thus, the device is capable of performing 10 parallel DETECTR reactions on a single sample.

FIG. 158 shows a possible design for an injection molded cartridge comprising one sample chamber connected to 4 amplification chamber, and 2 Detection chambers connected to each amplification chamber. The inj ection-molded cartridge comprises a series of valves and pumps or ports to pump manifolds that control flow throughout the cartridge.

FIG. 159 shows a possible design for an injection molded cartridge comprising one sample chamber connected to 4 amplification chamber, 2 Detection chambers connected to each amplification chamber, and a reagent chamber connected to the sample chamber.

FIG. 160 provides a top-down view of an injected-molded cartridge design with the reagent chambers in the flow paths leading to the amplification and Detection chambers.

FIG. 161 shows a portion of an injected-molded cartridge design with a sample chamber capable of connecting to multiple reagent and amplification chambers by a single rotating valve.

FIG. 162 shows a portion of an injected-molded cartridge design with a sliding valve connecting multiple compartments. Panels A-C show different positions that the sliding valve is capable of adopting.

FIG. 163 panel A shows a possible design for an injection-molded cartridge with a casing. Panel B provides a physical model of the design shown in panel A.

FIG. 164 panel A provides a bottom-up view a design of an injection-molded cartridge with a casing. Panel B provides a view of the top of the injection-molded cartridge.

FIG. 165 provides multiple views of an injection-molded cartridge with a sliding valve.

FIG. 166 provides two views of a portion of an injection-molded cartridge with multiple reagent wells that lead to transparent reaction chambers.

FIG. 167 panels A-B provide top-down views of an injection-molded cartridge design. Panel C shows a picture of a physical model of the injection-molded cartridge.

FIG. 168 shows a picture of an injection-molded cartridge housed in a device containing a diode array.

FIG. 169 shows a graphic user interface for controlling a device that contains an injection-molded cartridge and a diode array for detection.

FIG. 170 shows results from a series of fluorescence experiments utilizing an 8-diode detector array, an 8 chamber injection-molded cartridge, and dyes.

FIG. 171 shows fluorescence results from a series of HERC2 targeting DETECTR reactions and buffer controls, measured with an 8-diode detector array.

FIG. 172 shows an injection molded cartridge inserted into a device, with 8 chambers containing DETECTR reactions.

FIG. 173 shows the results of amplification of a SeraCare target nucleic acid using LAMP under different lysis conditions. Samples were amplified in a low pH buffer containing either buffer (top plots) or a viral lysis buffer (“VLB,” bottom plots). Buffers contained no reducing agent (“Control,” columns 1 and 4), Reducing Agent B (columns 2 and 5), or Reducing Agent A (columns 3 and 6). Samples were incubated for 5 minutes at either room temperature (left plots) or 95° C. (right plots). Samples containing either no target (“NTC”), 2.5, 25, or 250 copies per reaction. Assays were performed in triplicate using 5 μL of sample in a 25 μL reaction.

FIG. 174 shows the results of amplification of a SeraCare standard target nucleic acid using LAMP under different lysis conditions. Samples were amplified in a low pH buffer containing either buffer (left plots) or a viral lysis buffer (“VLB,” right plots). Buffers contained no reducing agent (“Control”), Reducing Agent B, or Reducing Agent A. Samples were incubated for 5 minutes at either room temperature (top plots) or 95° C. (bottom plots). Samples containing either no target (“NTC”), 1.5, 2.5, 15, 25, 150, or 250 copies per reaction. Assays were performed in triplicate using 3 μL of sample in a 15 μL reaction or 5 μL of sample in a 25 μL reaction.

FIG. 175 shows amplification of a SARS-CoV-2 N gene (“N”) and an RNase P sample input control nucleic acid (“RP”) in the presence of six different viral lysis buffers (“VLB,” “VLB-D,” “VLB-T,” “Buffer,” “Buffer-A,” and “Buffer-B”). Buffer-A contains Buffer with Reducing Agent A and Buffer-B contains Buffer with Reducing Agent B. Shaded squares indicate rate of amplification, with darker shading indicating faster amplification. Amplification was performed at either 95° C. (“95C”) or room temperature (“RT”) on high, medium, or low titer COVID-19 positive patient samples (“16.9,” “30.5,” and “33.6,” respectively). Samples were measured in duplicate.

FIG. 176 shows square wave voltammetry results for a DETECTR reaction performed with electroactive reporter nucleic acids. The results were collected immediately following (0 minutes) and 33 minutes after initiation of the DETECTR reaction.

FIG. 177 shows cyclic voltammetry results for a DETECTR reaction performed with electroactive reporter nucleic acids. The results were collected immediately following (0 minutes) and 26 minutes after initiation of the DETECTR reaction.

### DETAILED DESCRIPTION

The present disclosure provides various devices, systems, fluidic devices, and kits for rapid lab tests, which may quickly assess whether a target nucleic acid is present in a sample by using a programmable nuclease that can interact with functionalized surfaces of the fluidic systems to generate a detectable signal. In particular, provided herein are various devices, systems, fluidic devices, and kits for rapid lab tests, which may quickly assess whether a target nucleic acid is present in a biological sample. The target nucleic acid may be from a virus. For example, the devices, systems fluidic devices, and kits for rapid lab tests disclosed herein may assess whether a target nucleic acid from a strain of influenza virus is present in a sample. The influenza can be influenza A or influenza B. The virus may be a coronavirus. The compositions and methods provided herein disclose programmable nucleases that can be used in the systems, fluidic devices, and kits provided herein to detect target nucleic acids from influenza or another virus, for example another respiratory virus (e.g., coronavirus). In some embodiments, the target nucleic acids can be from an upper respiratory tract virus. In some embodiments, provided herein are devices, systems, fluidic devices, and kits that can perform multiplexed detection of more than one unique sequence of target nucleic acids. For example, the devices, systems, fluidic devices, kits, and programmable nucleases provided herein can be used for multiplexed detection of target nucleic acids from one or more than viruses. In particular embodiments, the devices, systems, fluidic devices, kits, and programmable nucleases provided herein can be used for multiplexed detection of influenza A and influenza B. In some embodiments, devices, systems, fluidic devices, kits, and programmable nucleases provided herein can be used for multiplexed detection of influenza A, influenza B, and one or more other viruses (e.g., coronavirus, RSV or another respiratory virus, such as an upper respiratory tract virus).

The systems and programmable nucleases disclosed herein can be used as a companion diagnostic with any of the diseases disclosed herein (e.g., RSV, sepsis, flu), or can be used in reagent kits, point-of-care diagnostics, or over-the-counter diagnostics. The systems may be used as a point of care diagnostic or as a lab test for detection of a target nucleic acid and, thereby, detection of a condition in a subject from which the sample was taken. The systems may be used to determine the presence or absence of a gene of interest (e.g., a gene associated with a disease state) in a subject from which the sample was taken. The systems may be used to determine the presence or absence of a pathogen (e.g., a virus or bacterium) in a subject from which the sample was taken. The systems may be used in various sites or locations, such as in laboratories, in hospitals, in physician offices/laboratories (POLs), in clinics, at remotes sites, or at home. Sometimes, the present disclosure provides various devices, systems, fluidic devices, and kits for consumer genetic use or for over the counter use.

Described herein are devices, systems, fluidic devices, kits, and methods for detecting the presence of a target nucleic acid in a sample. A target nucleic acid may be a gene, or a portion of a gene, associated with a disease state. A target nucleic acid may be a nucleic acid from a pathogen (e.g., a virus or a bacterium). The devices, systems, fluidic devices, kits, and methods for detecting the presence of a target nucleic acid in a sample can be used in a rapid lab tests for detection of a target nucleic acid of interest (e.g., target nucleic acids from influenza, coronavirus, or other pathogens, or target nucleic acids corresponding to a gene of interest). In particular, provided herein are devices, systems, fluidic devices, and kits, wherein the rapid lab tests can be performed in a single system. The target nucleic acid may be a portion of a nucleic acid from a virus or a bacterium or other agents responsible for a disease in the sample. The target nucleic acid may be a portion of an RNA or DNA from any organism in the sample. In some embodiments, programmable nucleases disclosed herein are activated to initiate trans cleavage activity of an RNA reporter by RNA or DNA. A programmable nuclease as disclosed herein is, in some cases, binds to a target RNA to initiate trans cleavage of an RNA reporter, and this programmable nuclease can be referred to as an RNA-activated programmable RNA nuclease. In some instances, a programmable nuclease as disclosed herein binds to a target DNA to initiate trans cleavage of an RNA reporter, and this programmable nuclease can be referred to as a DNA-activated programmable RNA nuclease. In some cases, a programmable nuclease as described herein is capable of being activated by a target RNA or a target DNA. For example, a Cas13 protein, such as Cas13a, disclosed herein is activated by a target RNA nucleic acid or a target DNA nucleic acid to transcollaterally cleave RNA reporter molecules. In some embodiments, the Cas13 binds to a target ssDNA which initiates trans cleavage of RNA reporters. The detection of the target nucleic acid in the sample may indicate the presence of the disease in the sample and may provide information for taking action to reduce the transmission of the disease to individuals in the disease-affected environment or near the disease-carrying individual. The detection of the target nucleic acid in the sample may indicate the presence of a disease mutation, such as a single nucleotide polymorphism (SNP) that provide antibiotic resistance to a disease-causing bacteria. The detection of the target nucleic acid is facilitated by a programmable nuclease. The programmable nuclease can become activated after binding of a guide nucleic acid with a target nucleic, in which the activated programmable nuclease can cleave the target nucleic acid and can have trans cleavage activity, which can also be referred to as “collateral” or “transcollateral” cleavage. Trans cleavage activity can be non-specific cleavage of nearby single-stranded nucleic acids by the activated programmable nuclease, such as trans cleavage of detector nucleic acids with a detection moiety. Once the detector nucleic acid is cleaved by the activated programmable nuclease, the detection moiety is released from the detector nucleic acid and generates a detectable signal that is immobilized to on a support medium. Often the detection moiety is at least one of a fluorophore, a dye, a polypeptide, or a nucleic acid. Sometimes the detection moiety binds to a capture molecule on the support medium to be immobilized. The detectable signal can be visualized on the support medium to assess the presence or level of the target nucleic acid associated with an ailment, such as a disease. The programmable nuclease can be a CRISPR-Cas (clustered regularly interspaced short palindromic repeats—CRISPR associated) nucleoprotein complex with trans cleavage activity, which can be activated by binding of a guide nucleic acid with a target nucleic acid.

In one aspect, described herein, is a system for detecting a target nucleic acid. The system may comprise a support medium; a guide nucleic acid targeting a target sequence; a programmable nuclease capable of being activated when complexed with the guide nucleic acid and the target sequence; and a single stranded detector nucleic acid comprising a detection moiety, wherein the detector nucleic acid is capable of being cleaved by the activated nuclease, thereby generating a first detectable signal.

In another aspect, described herein is a system for detecting a target nucleic acid, the system comprising a reagent chamber and a support medium for detection of the first detectable signal. The reagent chamber comprises a guide nucleic acid targeting a target sequence; a programmable nuclease capable of being activated when complexed with the guide nucleic acid and the target sequence; and a single stranded detector nucleic acid comprising a detection moiety, wherein the detector nucleic acid is capable of being cleaved by the activated nuclease, thereby generating a first detectable signal.

Further described herein is a method of detecting a target nucleic acid in a sample comprising contacting the sample with a guide nucleic acid targeting a target sequence, a programmable nuclease capable of being activated when complexed with the guide nucleic acid and the target sequence, a single stranded detector nucleic acid comprising a detection moiety, wherein the detector nucleic acid is capable of being cleaved by the activated nuclease, thereby generating a first detectable signal, and presenting the first detectable signal using a support medium.

Also described herein are various designs of assays for CRISPR-Cas diagnostics for detecting target nucleic acids (e.g., from influenza, coronavirus, or genes associated with a disease state). The design and format of the lateral flow assays disclosed herein can include new Cas reporter molecules, which can be tethered to the surface of the assay in a reaction chamber that is upstream of the lateral flow strip itself. The assay designs disclosed herein provide significant advantages as they minimize the chances of false positives, and thus can have improved sensitivity and specificity for a target nucleic acid.

Also described herein is a kit for detecting a target nucleic acid (e.g., from influenza, coronavirus, or genes associated with a disease state). The kit may comprise a support medium; a guide nucleic acid targeting a target sequence; a programmable nuclease capable of being activated when complexed with the guide nucleic acid and the target sequence; and a single stranded detector nucleic acid comprising a detection moiety, wherein the detector nucleic acid is capable of being cleaved by the activated nuclease, thereby generating a first detectable signal.

A biological sample from an individual or an environmental sample can be tested to determine whether the individual has a communicable disease. The biological sample can be tested to detect the presence or absence of at least one target nucleic acid from virus (e.g., an influenza virus, a coronavirus, or a respiratory syncytial virus). The biological sample can be tested to detect the presence or absence of at least one target nucleic acid from bacterium. The at least one target nucleic acid from a pathogen responsible for the disease that is detected can also indicate that the pathogen is wild-type or comprises a mutation that confers resistance to treatment, such as antibiotic treatment. In some embodiments, a biological sample from an individual or an environmental sample can be tested to determine whether the individual has a gene or gene mutation associated with a disease state. A sample from an individual or from an environment is applied to the reagents described herein. The reaction between the sample and the reagents may be performed in the reagent chamber provided in the kit or on a support medium provided in the kit. If the target nucleic acid is present in the sample, the target nucleic acid binds to the guide nucleic acid to activate the programmable nuclease. The activated programmable nuclease cleaves the detector nucleic acid and generates a detectable signal that can be visualized on the support medium. If the target nucleic acid is absent in the sample or below the threshold of detection, the guide nucleic acid remains unbound, the programmable nuclease remains inactivated, and the detector nucleic acid remains uncleaved. After the sample and the reagents are contacted for a predetermined time, the reacted sample is placed on a sample pad of a support medium. The sample can be placed on to the sample pad by dipping the support medium into the reagent chamber, applying the reacted sample to the sample pad, or allowing the sample to transport if the reagent was initially placed on the support medium. As the reacted sample and reagents move along the support medium to a detection region and after a predetermined amount of time after applying the reacted sample, a positive control marker can be visualized in the detection region. If the sample is positive for the target nucleic acid, a test marker for the detectable signal can also be visualized. The results in the detection region can be visualized by eye or using a mobile device. In some instances, an individual can open a mobile application for reading of the test results on a mobile device having a camera and take an image of the support medium, including the detection region, barcode, reference color scale, and fiduciary markers on the housing, using the camera of the mobile device and the graphic user interface (GUI) of the mobile application. The mobile application can identify the test, visualize the detection region in the image, and analyze to determine the presence or absence or the level of the target nucleic acid responsible for the disease. The mobile application can present the results of the test to the individual, store the test results in the mobile application, or communicate with a remote device and transfer the data of the test results.

Such devices, systems, fluidic devices, kits, and methods described herein may allow for detection of target nucleic acid, and in turn the viral infection (e.g., influenza viral infection, a coronavirus, or a respiratory syncytial virus), bacterial infection, or disease state associated with the target nucleic acid, in remote regions or low resource settings without specialized equipment. Also, such devices, systems, fluidic devices, kits, and methods described herein may allow for detection of target nucleic acid, and in turn the pathogen and disease associated with the target nucleic acid, in healthcare clinics or doctor offices without specialized equipment. In some cases, this provides a point of care testing for users to easily test for a disease or infection at home or quickly in an office of a healthcare provider. Assays that deliver results in under an hour, for example, in 15 to 60 minutes, are particularly desirable for at home testing for many reasons. Antivirals can be most effective when administered within the first 48 hours and improve antibiotic stewardship. Thus, the systems and assays disclosed herein, which are capable of delivering results in under an hour can will allow for the delivery of anti-viral therapy at an optimal time. Additionally, the systems and assays provided herein, which are capable of delivering quick diagnoses and results, can help keep or send a patient at home, improve comprehensive disease surveillance, and prevent the spread of an infection. In other cases, this provides a test, which can be used in a lab to detect a nucleic acid of interest in a sample from a subject. In particular, provided herein are devices, systems, fluidic devices, and kits, wherein the rapid lab tests can be performed in a single system. In some cases, this may be valuable in detecting diseases in a developing country and as a global healthcare tool to detect the spread of a disease or efficacy of a treatment or provide early detection of a viral infection, such as influenza.

Some methods as described herein use an editing technique, such as a technique using an editing enzyme or a programmable nuclease and guide nucleic acid, to detect a target nucleic acid. An editing enzyme or a programmable nuclease in the editing technique can be activated by a target nucleic acid, after which the activated editing enzyme or activated programmable nuclease can cleave nearby single-stranded nucleic acids, such detector nucleic acids with a detection moiety. A target nucleic acid (e.g., a target nucleic acid from a virus, such as influenza) can be amplified by isothermal amplification and then an editing technique can be used to detect the marker. In some instances, the editing technique can comprise an editing enzyme or programmable nuclease that, when activated, cleaves nearby RNA or DNA as the readout of the detection. The methods as described herein in some instances comprise obtaining a cell-free DNA sample, amplifying DNA from the sample, using an editing technique to cleave detector nucleic acids, and reading the output of the editing technique. In other instances, the method comprises obtaining a fluid sample from a patient, and without amplifying a nucleic acid of the fluid sample, using an editing technique to cleave detector nucleic acids, and detecting the nucleic acid. The method can also comprise using single-stranded detector DNA, cleaving the single-stranded detector DNA using an activated editing enzyme, wherein the editing enzyme cleaves at least 50% of a population of single-stranded detector DNA as measured by a change in color. A number of samples, guide nucleic acids, programmable nucleases or editing enzymes, support mediums, target nucleic acids, single-stranded detector nucleic acids, and reagents are consistent with the devices, systems, fluidic devices, kits, and methods disclosed herein.

Also disclosed herein are detector nucleic acids and methods detecting a target nucleic using the detector nucleic acids. Often, the detector nucleic acid is a protein-nucleic acid. For example, a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. Often, the protein-nucleic acid is an enzyme-nucleic acid or an enzyme substrate-nucleic acid. Sometimes, the protein-nucleic acid is attached to a solid support. The nucleic acid can be DNA, RNA, or a DNA/RNA hybrid. The methods described herein use a programmable nuclease, such as the CRISPR/Cas system, to detect a target nucleic acid. A method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

Cleavage of the protein-nucleic acid produces a signal. For example, cleavage of the protein-nucleic acid produces a calorimetric signal, a potentiometric signal, an amperometric signal, an optical signal, or a piezo-electric signal. Various devices can be used to detect these different types signals, which indicate whether a target nucleic acid is present in the sample.

### Sample

A number of samples are consistent with the devices, systems, fluidic devices, kits, and methods disclosed herein. These samples are, for example, consistent with fluidic devices disclosed herein for detection of a target nucleic acid within the sample, wherein the fluidic device may comprise multiple pumps, valves, reservoirs, and chambers for sample preparation, amplification of a target nucleic acid within the sample, mixing with a programmable nuclease, and detection of a detectable signal arising from cleavage of detector nucleic acids by the programmable nuclease within the fluidic system itself. These samples can comprise a target nucleic acid for detection of an ailment, such as a disease, pathogen, or virus, such as influenza. Generally, a sample from an individual or an animal or an environmental sample can be obtained to test for presence of a disease, or any mutation of interest. A biological sample from the individual may be blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. A tissue sample may be dissociated or liquefied prior to application to detection system of the present disclosure. Samples can comprise one or more target nucleic acids for detection of an ailment, such as a disease, cancer, or genetic disorder, or genetic information, such as for phenotyping, genotyping, or determining ancestry and are compatible with the reagents and support mediums as described herein. Generally, a sample can be taken from any place where a nucleic acid can be found. Samples can be taken from an individual/human, a non-human animal, or a crop, or an environmental sample can be obtained to test for presence of a disease, virus, pathogen, cancer, genetic disorder, or any mutation or pathogen of interest. A biological sample can be blood, serum, plasma, lung fluid, exhaled breath condensate, saliva, spit, urine, stool, feces, mucus, lymph fluid, peritoneal, cerebrospinal fluid, amniotic fluid, breast milk, gastric secretions, bodily discharges, secretions from ulcers, pus, nasal secretions, sputum, pharyngeal exudates, urethral secretions/mucus, vaginal secretions/mucus, anal secretion/mucus, semen, tears, an exudate, an effusion, tissue fluid, interstitial fluid (e.g., tumor interstitial fluid), cyst fluid, tissue, or, in some instances, a combination thereof. A sample can be an aspirate of a bodily fluid from an animal (e.g. human, animals, livestock, pet, etc.) or plant. A tissue sample can be from any tissue that may be infected or affected by a pathogen (e.g., a wart, lung tissue, skin tissue, and the like). A tissue sample (e.g., from animals, plants, or humans) can be dissociated or liquified prior to application to detection system of the present disclosure. A sample can be from a plant (e.g., a crop, a hydroponically grown crop or plant, and/or house plant). Plant samples can include extracellular fluid, from tissue (e.g., root, leaves, stem, trunk etc.). A sample can be taken from the environment immediately surrounding a plant, such as hydroponic fluid/water, or soil. A sample from an environment may be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system or be applied neat to the detection system. Sometimes, the sample is contained in no more 20 uL. The sample, in some cases, is contained in no more than 1, 5, 10, 15, 20, 25, 30, 35 40, 45, 50, 55, 60, 65, 70, 75, 80, 90, 100, 200, 300, 400, 500 uL, or any of value from 1 uL to 500 uL. Sometimes, the sample is contained in more than 500 uL.

In some instances, the sample is taken from single-cell eukaryotic organisms; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

The sample used for disease testing may comprise at least one target sequence that can bind to a guide nucleic acid of the reagents described herein. In some cases, the target sequence is a portion of a nucleic acid. A portion of a nucleic acid can be from a genomic locus, a transcribed mRNA, or a reverse transcribed cDNA. A portion of a nucleic acid can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A portion of a nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target sequence can be reverse complementary to a guide nucleic acid.

In some cases, the target sequence is a portion of a nucleic acid from a virus or a bacterium or other agents responsible for a disease in the sample. The target sequence, in some cases, is a portion of a nucleic acid from a sexually transmitted infection or a contagious disease, in the sample. The target sequence, in some cases, is a portion of a nucleic acid from an upper respiratory tract infection, a lower respiratory tract infection, or a contagious disease, in the sample. The target sequence, in some cases, is a portion of a nucleic acid from a hospital acquired infection or a contagious disease, in the sample. The target sequence, in some cases, is an ssRNA. These target sequences may be from a disease, and the disease may include but is not limited to influenza virus including influenza A virus (IAV) or influenza B virus (IBV), rhinovirus, cold viruses, a respiratory virus, an upper respiratory virus, a lower respiratory virus, or respiratory syncytial virus. Pathogens include viruses, fungi, helminths, protozoa, and parasites. Pathogenic viruses include but are not limited to influenza virus and the like. Pathogens include, e.g., Mycobacterium tuberculosis, Streptococcus agalactiae, methicillin-resistant Staphylococcus aureus, Legionella pneumophila, Streptococcus pyogenes, Escherichia coli, Neisseria meningitidis, Pneumococcus, Hemophilus influenzae B, influenza virus, respiratory syncytial virus (RSV), M. pneumoniae, Streptococcus intermdius, Streptococcus pneumoniae, and Streptococcus pyogenes. Often the target nucleic acid comprises a sequence from a virus or a bacterium or other agents responsible for a disease that can be found in the sample. Pathogenic viruses include but are not limited to influenza virus; RSV; coronavirus, an ssRNA virus, a respiratory virus, an upper respiratory virus, a lower respiratory virus, or a rhinovirus. Pathogens include, e.g., Mycobacterium tuberculosis, Streptococcus agalactiae, Legionella pneumophila, Streptococcus pyogenes, Hemophilus influenzae B influenza virus, respiratory syncytial virus (RSV), or Mycobacterium tuberculosis

In some cases, the target sequence is a portion of a nucleic acid from a virus or a bacterium or other agents responsible for a disease in the sample. The target sequence, in some cases, is a portion of a nucleic acid from a sexually transmitted infection or a contagious disease, in the sample. The target sequence, in some cases, is a portion of a nucleic acid from an upper respiratory tract infection, a lower respiratory tract infection, or a contagious disease, in the sample. The target sequence, in some cases, is a portion of a nucleic acid from a hospital acquired infection or a contagious disease, in the sample. The target sequence, in some cases, is a portion of a nucleic acid from sepsis, in the sample. These diseases can include but are not limited to respiratory viruses (e.g., COVID-19, SARS, MERS, influenza and the like) human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitides, Chlamydia trachomatis, and Candida albicans. Pathogenic viruses include but are not limited to: respiratory viruses (e.g., adenoviruses, parainfluenza viruses, severe acute respiratory syndrome (SARS), coronavirus, MERS), gastrointestinal viruses (e.g., noroviruses, rotaviruses, some adenoviruses, astroviruses), exanthematous viruses (e.g. the virus that causes measles, the virus that causes rubella, the virus that causes chickenpox/shingles, the virus that causes roseola, the virus that causes smallpox, the virus that causes fifth disease, chikungunya virus infection); hepatic viral diseases (e.g., hepatitis A, B, C, D, E); cutaneous viral diseases (e.g. warts (including genital, anal), herpes (including oral, genital, anal), molluscum contagiosum); hemmorhagic viral diseases (e.g. Ebola, Lassa fever, dengue fever, yellow fever, Marburg hemorrhagic fever, Crimean-Congo hemorrhagic fever); neurologic viruses (e.g., polio, viral meningitis, viral encephalitis, rabies), sexually transmitted viruses (e.g., HIV, HPV, and the like), immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Klebsiella pneumoniae, Acinetobacter baumannii, Burkholderia cepacia, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. Vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Enterobacter cloacae, Kiebsiella aerogenes, Proteus vulgaris, Serratia macesens, Enterococcus faecalis, Enterococcus faecium, Streptococcus intermdius, Streptococcus pneumoniae, and Streptococcus pyogenes. Often the target nucleic acid comprises a sequence from a virus or a bacterium or other agents responsible for a disease that can be found in the sample. In some cases, the target nucleic acid is a portion of a nucleic acid from a genomic locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitides, Chlamydia trachomatis, and Candida albicans. Pathogenic viruses include but are not limited to immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment.

The sample used for cancer testing or cancer risk testing can comprise at least one target nucleic acid segment that can bind to a guide nucleic acid of the reagents described herein. The target nucleic acid segment, in some cases, is a portion of a nucleic acid from a gene with a mutation associated with cancer, from a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes for a cancer biomarker, such as a prostate cancer biomarker or non-small cell lung cancer. In some cases, the assay can be used to detect “hotspots” in target nucleic acids that can be predictive of cancer, such as lung cancer, cervical cancer, in some cases, the cancer can be a cancer that is caused by a virus. Some non-limiting examples of viruses that cause cancers in humans include Epstein-Barr virus (e.g., Burkitt's lymphoma, Hodgkin's Disease, and nasopharyngeal carcinoma); papillomavirus (e.g., cervical carcinoma, anal carcinoma, oropharyngeal carcinoma, penile carcinoma); hepatitis B and C viruses (e.g., hepatocellular carcinoma); human adult T-cell leukemia virus type 1 (HTLV-1) (e.g., T-cell leukemia); and Merkel cell polyomavirus (e.g., Merkel cell carcinoma). One skilled in the art will recognize that viruses can cause or contribute to other types of cancers. In some cases, the target nucleic acid is a portion of a nucleic acid that is associated with a blood fever. In some cases, the target nucleic acid segment is a portion of a nucleic acid from a genomic locus, a transcribed mRNA, or a reverse transcribed cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, KIT, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1.

The sample used for genetic disorder testing can comprise at least one target nucleic acid segment that can bind to a guide nucleic acid of the reagents described herein. In some embodiments, the genetic disorder is hemophilia, sickle cell anemia, β-thalassemia, Duchene muscular dystrophy, severe combined immunodeficiency, or cystic fibrosis. The target nucleic acid segment, in some cases, is a portion of a nucleic acid from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid segment is a portion of a nucleic acid from a genomic locus, a transcribed mRNA, or a reverse transcribed cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRE1C, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

In some embodiments, the target nucleic acid sequence comprises a nucleic acid sequence of a virus, a bacterium, or other pathogen responsible for a disease in a plant (e.g., a crop). Methods and compositions of the disclosure can be used to treat or detect a disease in a plant. For example, the methods of the disclosure can be used to target a viral nucleic acid sequence in a plant. A programmable nuclease of the disclosure can cleave the viral nucleic acid. In some embodiments, the target nucleic acid sequence comprises a nucleic acid sequence of a virus or a bacterium or other agents (e.g., any pathogen) responsible for a disease in the plant (e.g., a crop). In some embodiments, the target nucleic acid comprises DNA that is reverse transcribed from RNA using a reverse transcriptase prior to detection by a programmable nuclease using the compositions, systems, and methods disclosed herein. The target nucleic acid, in some cases, is a portion of a nucleic acid from a virus or a bacterium or other agents responsible for a disease in the plant (e.g., a crop). In some cases, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant can be an RNA virus. A virus infecting the plant can be a DNA virus. Non-limiting examples of viruses that can be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

The plant can be a monocotyledonous plant. The plant can be a dicotyledonous plant. Non-limiting examples of orders of dicotyledonous plants include Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales.

Non-limiting examples of orders of monocotyledonous plants include Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales. A plant can belong to the order, for example, Gymnospermae, Pinales, Ginkgoales, Cycadales, Araucariales, Cupressales and Gnetales.

Non-limiting examples of plants include plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses, wheat, maize, rice, millet, barley, tomato, apple, pear, strawberry, orange, acacia, carrot, potato, sugar beets, yam, lettuce, spinach, sunflower, rape seed, Arabidopsis, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. A plant can include algae.

The sample can be used for identifying a disease status. For example, a sample is any sample described herein, and is obtained from a subject for use in identifying a disease status of a subject. Sometimes, a method comprises obtaining a serum sample from a subject; and identifying a disease status of the subject.

In some instances, the target nucleic acid is a single stranded nucleic acid. Alternatively or in combination, the target nucleic acid is a double stranded nucleic acid and is prepared into single stranded nucleic acids before or upon contacting the reagents. The target nucleic acid may be a RNA, DNA, synthetic nucleic acids, or nucleic acids found in biological or environmental samples. The target nucleic acids include but are not limited to mRNA, rRNA, tRNA, non-coding RNA, long non-coding RNA, and microRNA (miRNA). In some cases, the target nucleic acid is mRNA. In some cases, the target nucleic acid is from a virus, a parasite, or a bacterium described herein. In some cases, the target nucleic acid is transcribed from a gene as described herein.

A number of target nucleic acids are consistent with the methods and compositions disclosed herein. Some methods described herein can detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the method detects target nucleic acid present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

A number of target nucleic acid populations are consistent with the methods and compositions disclosed herein. Some methods described herein can detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the method detects target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations can be present at different concentrations or amounts in the sample.

Any of the above disclosed samples are consistent with the systems, assays, and programmable nucleases disclosed herein and can be used as a companion diagnostic with any of the diseases disclosed herein (e.g., influenza A, influenza B, RSV), or can be used in reagent kits, point-of-care diagnostics, or over-the-counter diagnostics.

Reagents A number of reagents are consistent with the devices, systems, fluidic devices, kits, and methods disclosed herein. These reagents are, for example, consistent for use within various fluidic devices disclosed herein for detection of a target nucleic acid (e.g., influenza A or influenza B) within the sample, wherein the fluidic device may comprise multiple pumps, valves, reservoirs, and chambers for sample preparation, amplification of a target nucleic acid within the sample, mixing with a programmable nuclease, and detection of a detectable signal arising from cleavage of detector nucleic acids by the programmable nuclease within the fluidic system itself. These reagents are compatible with the samples, fluidic devices, and support mediums as described herein for detection of an ailment, such as a disease. The reagents described herein for detecting a disease, such as influenza or RSV, comprise a guide nucleic acid targeting the target nucleic acid segment indicative of the disease. The guide nucleic acid binds to the single stranded target nucleic acid comprising a portion of a nucleic acid from a virus or a bacterium or other agents responsible for a disease as described herein. The guide nucleic acid can bind to the single stranded target nucleic acid comprising a portion of a nucleic acid from a bacterium or other agents responsible for a disease as described herein and further comprising a mutation, such as a single nucleotide polymorphism (SNP), that can confer resistance to a treatment, such as antibiotic treatment. The guide nucleic acid binds to the single stranded target nucleic acid comprising a portion of a nucleic acid from an influenza virus, such as influenza A or influenza B. The guide nucleic acid is complementary to the target nucleic acid. Often the guide nucleic acid binds specifically to the target nucleic acid. The target nucleic acid may be a RNA, DNA, or synthetic nucleic acids.

Disclosed herein are methods of assaying for a target nucleic acid as described herein. For example, a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

A programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby single-stranded nucleic acids by the activated programmable nuclease, such as trans cleavage of detector nucleic acids with a detection moiety. Once the detector nucleic acid is cleaved by the activated programmable nuclease, the detection moiety can be released from the detector nucleic acid and can generate a signal. A signal can be a calorimetric, potentiometric, amperometric, optical (e.g., fluorescent, colorometric, etc.), or piezo-electric signal. Often, the signal is present prior to detector nucleic acid cleavage and changes upon detector nucleic acid cleavage. Sometimes, the signal is absent prior to detector nucleic acid cleavage and is present upon detector nucleic acid cleavage. The detectable signal can be immobilized on a support medium for detection. The programmable nuclease can be a CRISPR-Cas (clustered regularly interspaced short palindromic repeats—CRISPR associated) nucleoprotein complex with trans cleavage activity, which can be activated by binding of a guide nucleic acid with a target nucleic acid. The CRISPR-Cas nucleoprotein complex can comprise a Cas protein (also referred to as a Cas nuclease) complexed with a guide nucleic acid, which can also be referred to as CRISPR enzyme. A guide nucleic acid can be a CRISPR RNA (crRNA). Sometimes, a guide nucleic acid comprises a crRNA and a trans-activating crRNA (tracrRNA).

The CRISPR/Cas system used to detect a modified target nucleic acids can comprise CRISPR RNAs (crRNAs), trans-activating crRNAs (tracrRNAs), Cas proteins, and detector nucleic acids.

A guide nucleic acid can comprise a sequence that is reverse complementary to the sequence of a target nucleic acid. A guide nucleic acid can be a crRNA. Sometimes, a guide nucleic acid comprises a crRNA and tracrRNA. The guide nucleic acid can bind specifically to the target nucleic acid. In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. The target nucleic acid can be designed and made to provide desired functions. In some cases, the targeting region of a guide nucleic acid is 20 nucleotides in length. The targeting region of the guide nucleic acid may have a length of at least 10, 11, 12, 13, 14, 15, 16, 17, 18, 19, 20, 21, 22, 23, 24, 25, 26, 27, 28, 29, or 30 nucleotides in length. In some instances, the targeting region of the guide nucleic acid is 10, 11, 12, 13, 14, 15, 16, 17, 18, 19, 20, 21, 22, 23, 24, 25, 26, 27, 28, 29, or 30 nucleotides in length. In some cases, the targeting region of a guide nucleic acid has a length from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. It is understood that the sequence of a polynucleotide need not be 100% complementary to that of its target nucleic acid to be specifically hybridizable or hybridizable or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid.

The guide nucleic acid can be selected from a group of guide nucleic acids that have been tiled against the nucleic acid sequence of a strain of an infection or genomic locus of interest. The guide nucleic acid can be selected from a group of guide nucleic acids that have been tiled against the nucleic acid sequence of a strain of influenza A or influenza B. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling comprises gaps between the tiled guide nucleic acids along the target nucleic acid. In some instances the tiling of the guide nucleic acids is non-sequential. Often, a method for detecting a target nucleic acid comprises contacting a target nucleic acid to a pool of guide nucleic acids and a programmable nuclease, wherein a guide nucleic acid of the pool of guide nucleic acids has a sequence selected from a group of tiled guide nucleic acid that correspond to nucleic acids of a target nucleic acid; and assaying for a signal produce by cleavage of at least some detector nucleic acids of a population of detector nucleic acids. Pooling of guide nucleic acids can ensure broad spectrum identification, or broad coverage, of a target species within a single reaction. This can be particularly helpful in diseases or indications, like sepsis, that may be caused by multiple organisms.

Described herein are reagents comprising a programmable nuclease capable of being activated when complexed with the guide nucleic acid and the target nucleic acid segment. A programmable nuclease can be capable of being activated when complexed with a guide nucleic acid and the target sequence. The programmable nuclease can be activated upon binding of the guide nucleic acid to its target nucleic acid and degrades non-specifically nucleic acid in its environment. The programmable nuclease has trans cleavage activity once activated. A programmable nuclease can be a Cas protein (also referred to, interchangeably, as a Cas nuclease). A crRNA and Cas protein can form a CRISPR enzyme.

“Percent identity” and “% identity” can refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

Several programmable nucleases are consistent with the methods and devices of the present disclosure. For example, CRISPR/Cas enzymes are programmable nucleases used in the methods and systems disclosed herein. CRISPR/Cas enzymes can include any of the known Classes and Types of CRISPR/Cas enzymes. Programmable nucleases disclosed herein include Class 1 CRISPR/Cas enzymes, such as the Type I, Type IV, or Type III CRISPR/Cas enzymes. Programmable nucleases disclosed herein also include the Class 2 CRISPR/Cas enzymes, such as the Type II, Type V, and Type VI CRISPR/Cas enzymes. Preferable programmable nucleases included in the several devices disclosed herein (e.g., a microfluidic device such as a pneumatic valve device or a sliding valve device or a lateral flow assay) and methods of use thereof include a Type V or Type VI CRISPR/Cas enzyme.

In some embodiments, the Type V CRISPR/Cas enzyme is a programmable Cas12 nuclease. Type V CRISPR/Cas enzymes (e.g., Cas12 or Cas14) lack an HNH domain. A Cas12 nuclease of the present disclosure cleaves a nucleic acids via a single catalytic RuvC domain. The RuvC domain is within a nuclease, or “NUC” lobe of the protein, and the Cas12 nucleases further comprise a recognition, or “REC” lobe. The REC and NUC lobes are connected by a bridge helix and the Cas12 proteins additionally include two domains for PAM recognition termed the PAM interacting (PI) domain and the wedge (WED) domain. (Murugan et al., Mol Cell. 2017 Oct. 5; 68(1): 15-25). A programmable Cas12 nuclease can be a Cas12a (also referred to as Cpf1) protein, a Cas12b protein, Cas12c protein, Cas12d protein, or a Cas12e protein. In some cases, a suitable Cas12 protein comprises an amino acid sequence having at least 80%, at least 85%, at least 90%, at least 95%, at least 98%, at least 99%, or 100%, amino acid sequence identity to any one of SEQ ID NO: 27-SEQ ID NO: 37.

TABLE 1
Cas12 Protein Sequences

| SEQ | | |
|---|---|---|
| ID | | |
| NO | Description | Sequence |
| SEQ | Lachnospiraceae | MSKLEKFTNCYSLSKTLRFKAIPVGKTQENIDNKRLLVEDEKRAEDYK |
| ID | bacterium | GVKKLLDRYYLSFINDVLHSIKLKNLNNYISLFRKKTRTEKENKELENL |
| NO: | ND2006 | EINLRKEIAKAFKGNEGYKSLFKKDIIETILPEFLDDKDEIALVNSFNGFT |
| 27 | (LbCas12a) | TAFTGFFDNRENMFSEEAKSTSIAFRCINENLTRYISNMDIFEKVDAIFD |
| | | KHEVQEIKEKILNSDYDVEDFFEGEFFNFVLTQEGIDVYNAIIGGFVTES |
| | | GEKIKGLNEYINLYNQKTKQKLPKFKPLYKQVLSDRESLSFYGEGYTSD |
| | | EEVLEVFRNTLNKNSEIFSSIKKLEKLFKNFDEYSSAGIFVKNGPAISTIS |
| | | KDIFGEWNVIRDKWNAEYDDIHLKKKAVVTEKYEDDRRKSFKKIGSFS |
| | | LEQLQEYADADLSVVEKLKEIIIQKVDEIYKVYGSSEKLFDADFVLEKSL |
| | | KKNDAVVAIMKDLLDSVKSFENYIKAFFGEGKETNRDESFYGDFVLAY |
| | | DILLKVDHIYDAIRNYVTQKPYSKDKFKLYFQNPQFMGGWDKDKETD |
| | | YRATILRYGSKYYLAIMDKKYAKCLQKIDKDDVNGNYEKINYKLLPGP |
| | | NKMLPKVFFSKKWMAYYNPSEDIQKIYKNGTFKKGDMFNLNDCHKLI |
| | | DFFKDSISRYPKWSNAYDFNFSETEKYKDIAGFYREVEEQGYKVSFESA |
| | | SKKEVDKLVEEGKLYMFQIYNKDFSDKSHGTPNLHTMYFKLLFDENN |
| | | HGQIRLSGGAELFMRRASLKKEELVVHPANSPIANKNPDNPKKTTTLSY |
| | | DVYKDKRFSEDQYELHIPIAINKCPKNIFKINTEVRVLLKHDDNPYVIGI |
| | | DRGERNLLYIVVVDGKGNIVEQYSLNEIINNFNGIRIKTDYHSLLDKKEK |
| | | ERFEARQNWTSIENIKELKAGYISQVVHKICELVEKYDAVIALEDLNSG |
| | | FKNSRVKVEKQVYQKFEKMLIDKLNYMVDKKSNPCATGGALKGYQIT |
| | | NKFESFKSMSTQNGFIFYIPAWLTSKIDPSTGFVNLLKTKYTSIADSKKFI |
| | | SSFDRIMYVPEEDLFEFALDYKNFSRTDADYIKKWKLYSYGNRIRIFRN |
| | | PKKNNVFDWEEVCLTSAYKELFNKYGINYQQGDIRALLCEQSDKAFYS |
| | | SFMALMSLMLQMRNSITGRTDVDFLISPVKNSDGIFYDSRNYEAQENAI |
| | | LPKNADANGAYNIARKVLWAIGQFKKAEDEKLDKVKIAISNKEWLEY |
| | | AQTSVKH |
| SEQ | Acidaminococcus | MTQFEGFTNLYQVSKTLRFELIPQGKTLKHIQEQGFIEEDKARNDHYKE |
| ID | sp. | LKPIIDRIYKTYADQCLQLVQLDWENLSAAIDSYRKEKTEETRNALIEEQ |
| NO: | BV316 | ATYRNAIHDYFIGRTDNLTDAINKRHAEIYKGLFKAELFNGKVLKQLGT |
| 28 | (AsCasl2a) | VTTTEHENALLRSFDKFTTYFSGFYENRKNVFSAEDISTAIPHRIVQDNF |
| | | PKFKENCHIFTRLITAVPSLREHFENVKKAIGIFVSTSIEEVFSFPFYNQLL |
| | | TQTQIDLYNQLLGGISREAGTEKIKGLNEVLNLAIQKNDETAHIIASLPH |
| | | RFIPLFKQILSDRNTLSFILEEFKSDEEVIQSFCKYKTLLRNENVLETAEA |
| | | LFNELNSIDLTHIFISHKKLETISSALCDHWDTLRNALYERRISELTGKIT |
| | | KSAKEKVQRSLKHEDINLQEIISAAGKELSEAFKQKTSEILSHAHAALDQ |
| | | PLPTTLKKQEEKEILKSQLDSLLGLYHLLDWFAVDESNEVDPEFSARLT |
| | | GIKLEMEPSLSFYNKARNYATKKPYSVEKFKLNFQMPTLASGWDVNKE |
| | | KNNGAILFVKNGLYYLGIMPKQKGRYKALSFEPTEKTSEGFDKMYYDY |
| | | FPDAAKMIPKCSTQLKAVTAHFQTHTTPILLSNNFIEPLEITKEIYDLNNP |
| | | EKEPKKFQTAYAKKTGDQKGYREALCKWIDFTRDFLSKYTKTTSIDLSS |
| | | LRPSSQYKDLGEYYAELNPLLYHISFQRIAEKEIMDAVETGKLYLFQIYN |
| | | KDFAKGHHGKPNLHTLYWTGLFSPENLAKTSIKLNGQAELFYRPKSRM |
| | | KRMAHRLGEKMLNKKLKDQKTPIPDTLYQELYDYVNHRLSHDLSDEA |
| | | RALLPNVITKEVSHEIIKDRRFTSDKFFFHVPITLNYQAANSPSKFNQRV |
| | | NAYLKEHPETPIIGIDRGERNLIYITVIDSTGKILEQRSLNTIQQFDYQKKL |
| | | DNREKERVAARQAWSVVGTIKDLKQGYLSQVIHEIVDLMIHYQAVVV |
| | | LENLNFGFKSKRTGIAEKAVYQQFEKMLIDKLNCLVLKDYPAEKVGGV |
| | | LNPYQLTDQFTSFAKMGTQSGFLFYVPAPYTSKIDPLTGFVDPFVWKTI |
| | | KNHESRKHFLEGFDFLHYDVKTGDFILHFKMNRNLSFQRGLPGFMPAW |
| | | DIVFEKNETQFDAKGTPFIAGKRIVPVIENHRFTGRYRDLYPANELIALL |
| | | EEKGIVFRDGSNILPKLLENDDSHAIDTMVALIRSVLQMRNSNAATGED |
| | | YINSPVRDLNGVCFDSRFQNPEWPMDADANGAYHIALKGQLLLNHLK |
| | | ESKDLKLQNGISNQDWLAYIQELRN |
| SEQ | Francisella | MSIYQEFVNKYSLSKTLRFELIPQGKTLENIKARGLILDDEKRAKDYKK |
| ID | novicida | AKQIIDKYHQFFIEEILSSVCISEDLLQNYSDVYFKLKKSDDDNLQKDFK |
| NO: | UH2 | SAKDTIKKQISEYIKDSEKFKNLFNQNLIDAKKGQESDLILWLKQSKDN |
| 29 | (FnCas12a) | GIELFKANSDITDIDEALEIIKSFKGWTTYFKGFHENRKNVYSSNDIPTSII |
| | | YRIVDDNLPKFLENKAKYESLKDKAPEAINYEQIKKDLAEELTFDIDYK |
| | | TSEVNQRVFSLDEVFEIANFNNYLNQSGITKFNTIIGGKFVNGENTKRKG |
| | | INEYINLYSQQINDKTLKKYKMSVLFKQILSDTESKSFVIDKLEDDSDVV |
| | | TTMQSFYEQIAAFKTVEEKSIKETLSLLFDDLKAQKLDLSKIYFKNDKSL |
| | | TDLSQQVFDDYSVIGTAVLEYITQQIAPKNLDNPSKKEQELIAKKTEKA |
| | | KYLSLETIKLALEEFNKHRDIDKQCRFEEILANFAAIPMIFDEIAQNKDN |
| | | LAQISIKYQNQGKKDLLQASAEDDVKAIKDLLDQTNNLLHKLKIFHISQ |
| | | SEDKANILDKDEHFYLVFEECYFELANIVPLYNKIRNYITQKPYSDEKFK |
| | | LNFENSTLANGWDKNKEPDNTAILFIKDDKYYLGVMNKKNNKIFDDK |
| | | AIKENKGEGYKKIVYKLLPGANKMLPKVFFSAKSIKFYNPSEDILRIRNH |
| | | STHTKNGSPQKGYEKFEFNIEDCRKFIDFYKQSISKHPEWKDFGFRFSDT |
| | | QRYNSIDEFYREVENQGYKLTFENISESYIDSVVNQGKLYLFQIYNKDFS |
| | | AYSKGRPNLHTLYWKALFDERNLQDVVYKLNGEAELFYRKQSIPKKIT |
| | | HPAKEAIANKNKDNPKKESVFEYDLIKDKRFTEDKFFFHCPITINFKSSG |
| | | ANKFNDEINLLLKEKANDVHILSIDRGERHLAYYTLVDGKGNIIKQDTF |
| | | NIIGNDRMKTNYHDKLAAIEKDRDSARKDWKKINNIKEMKEGYLSQV |
| | | VHEIAKLVIEYNAIVVFEDLNFGFKRGRFKVEKQVYQKLEKMLIEKLNY |
| | | LVFKDNEFDKTGGVLRAYQLTAPFETFKKMGKQTGIIYYVPAGFTSKIC |
| | | PVTGFVNQLYPKYESVSKSQEFFSKFDKICYNLDKGYFEFSFDYKNFGD |
| | | KAAKGKWTIASFGSRLINFRNSDKNHNWDTREVYPTKELEKLLKDYSI |
| | | EYGHGECIKAAICGESDKKFFAKLTSVLNTILQMRNSKTGTELDYLISPV |
| | | ADVNGNFFDSRQAPKNMPQDADANGAYHIGLKGLMLLGRIKNNQEGK |
| | | KLNLVIKNEEYFEFVQNRNN |
| SEQ | Porphyromonas | MKTQHFFEDFTSLYSLSKTIRFELKPIGKTLENIKKNGLIRRDEQRLDDY |
| ID | macacae | EKLKKVIDEYHEDFIANILSSFSFSEEILQSYIQNLSESEARAKIEKTMRD |
| NO: | (PmCas12a) | TLAKAFSEDERYKSIFKKELVKKDIPVWCPAYKSLCKKFDNFTTSLVPF |
| 30 | | HENRKNLYTSNEITASIPYRIVHVNLPKFIQNIEALCELQKKMGADLYLE |
| | | MMENLRNVWPSFVKTPDDLCNLKTYNHLMVQSSISEYNRFVGGYSTE |
| | | DGTKHQGINEWINIYRQRNKEMRLPGLVFLHKQILAKVDSSSFISDTLE |
| | | NDDQVFCVLRQFRKLFWNTVSSKEDDAASLKDLFCGLSGYDPEAIYVS |
| | | DAHLATISKNIFDRWNYISDAIRRKTEVLMPRKKESVERYAEKISKQIKK |
| | | RQSYSLAELDDLLAHYSEESLPAGFSLLSYFTSLGGQKYLVSDGEVILY |
| | | EEGSNIWDEVLIAFRDLQVILDKDFTEKKLGKDEEAVSVIKKALDSALR |
| | | LRKFFDLLSGTGAEIRRDSSFYALYTDRMDKLKGLLKMYDKVRNYLTK |
| | | KPYSIEKFKLHFDNPSLLSGWDKNKELNNLSVIFRQNGYYYLGIMTPKG |
| | | KNLFKTLPKLGAEEMFYEKMEYKQIAEPMLMLPKVFFPKKTKPAFAPD |
| | | QSVVDIYNKKTFKTGQKGFNKKDLYRLIDFYKEALTVHEWKLFNFSFS |
| | | PTEQYRNIGEFFDEVREQAYKVSMVNVPASYIDEAVENGKLYLFQIYN |
| | | KDFSPYSKGIPNLHTLYWKALFSEQNQSRVYKLCGGGELFYRKASLHM |
| | | QDTTVHPKGISIHKKNLNKKGETSLFNYDLVKDKRFTEDKFFFHVPISIN |
| | | YKNKKITNVNQMVRDYIAQNDDLQIIGIDRGERNLLYISRIDTRGNLLE |
| | | QFSLNVIESDKGDLRTDYQKILGDREQERLRRRQEWKSIESIKDLKDGY |
| | | MSQVVHKICNMVVEHKAIVVLENLNLSFMKGRKKVEKSVYEKFERML |
| | | VDKLNYLVVDKKNLSNEPGGLYAAYQLTNPLFSFEELHRYPQSGILFFV |
| | | DPWNTSLTDPSTGFVNLLGRINYTNVGDARKFFDRFNAIRYDGKGNILF |
| | | DLDLSRFDVRVETQRKLWTLTTFGSRIAKSKKSGKWMVERIENLSLCFL |
| | | ELFEQFNIGYRVEKDLKKAILSQDRKEFYVRLIYLFNLMMQIRNSDGEE |
| | | DYILSPALNEKNLQFDSRLIEAKDLPVDADANGAYNVARKGLMVVQRI |
| | | KRGDHESIHRIGRAQWLRYVQEGIVE |
| SEQ | Moraxella | MLFQDFTHLYPLSKTVRFELKPIDRTLEHIHAKNFLSQDETMADMHQK |
| ID | bovoculi | VKVILDDYHRDFIADMMGEVKLTKLAEFYDVYLKFRKNPKDDELQKQ |
| NO: | 237 | LKDLQAVLRKEIVKPIGNGGKYKAGYDRLFGAKLFKDGKELGDLAKF |
| 31 | (MbCas12a) | VIAQEGESSPKLAHLAHFEKFSTYFTGFHDNRKNMYSDEDKHTAIAYR |
| | | LIHENLPRFIDNLQILTTIKQKHSALYDQIINELTASGLDVSLASHLDGYH |
| | | KLLTQEGITAYNTLLGGISGEAGSPKIQGINELINSHHNQHCHKSERIAK |
| | | LRPLHKQILSDGMSVSFLPSKFADDSEMCQAVNEFYRHYADVFAKVQS |
| | | LFDGFDDHQKDGIYVEHKNLNELSKQAFGDFALLGRVLDGYYVDVVN |
| | | PEFNERFAKAKTDNAKAKLTKEKDKFIKGVHSLASLEQAIEHYTARHD |
| | | DESVQAGKLGQYFKHGLAGVDNPIQKIHNNHSTIKGFLERERPAGERA |
| | | LPKIKSGKNPEMTQLRQLKELLDNALNVAHFAKLLTTKTTLDNQDGNF |
| | | YGEFGVLYDELAKIPTLYNKVRDYLSQKPFSTEKYKLNFGNPTLLNGW |
| | | DLNKEKDNFGVILQKDGCYYLALLDKAHKKVFDNAPNTGKSIYQKMI |
| | | YKYLEVRKQFPKVFFSKEAIAINYHPSKELVEIKDKGRQRSDDERLKLY |
| | | RFILECLKIHPKYDKKFEGAIGDIQLFKKDKKGREVPISEKDLFDKINGIF |
| | | SSKPKLEMEDFFIGEFKRYNPSQDLVDQYNIYKKIDSNDNRKKENFYNN |
| | | HPKFKKDLVRYYYESMCKHEEWEESFEFSKKLQDIGCYVDVNELFTEI |
| | | ETRRLNYKISFCNINADYIDELVEQGQLYLFQIYNKDFSPKAHGKPNLH |
| | | TLYFKALFSEDNLADPIYKLNGEAQIFYRKASLDMNETTIHRAGEVLEN |
| | | KNPDNPKKRQFVYDIIKDKRYTQDKFMLHVPITMNFGVQGMTIKEFNK |
| | | KVNQSIQQYDEVNVIGIDRGERHLLYLTVINSKGEILEQCSLNDITTASA |
| | | NGTQMTTPYHKILDKREIERLNARVGWGEIETIKELKSGYLSHVVHQIS |
| | | QLMLKYNAIVVLEDLNFGFKRGRFKVEKQIYQNFENALIKKLNHLVLK |
| | | DKADDEIGSYKNALQLTNNFTDLKSIGKQTGFLFYVPAWNTSKIDPETG |
| | | FVDLLKPRYENIAQSQAFFGKFDKICYNADKDYFEFHIDYAKFTDKAK |
| | | NSRQIWTICSHGDKRYVYDKTANQNKGAAKGINVNDELKSLFARHHIN |
| | | EKQPNLVMDICQNNDKEFHKSLMYLLKTLLALRYSNASSDEDFILSPVA |
| | | NDEGVFFNSALADDTQPQNADANGAYHIALKGLWLLNELKNSDDLNK |
| | | VKLAIDNQTWLNFAQNR |
| SEQ | Moraxella | MGIHGVPAALFQDFTHLYPLSKTVRFELKPIGRTLEHIHAKNFLSQDET |
| ID | bovoculi | MADMYQKVKVILDDYHRDFIADMMGEVKLTKLAEFYDVYLKFRKNP |
| NO: | AAX08_00205 | KDDGLQKQLKDLQAVLRKESVKPIGSGGKYKTGYDRLFGAKLFKDGK |
| 32 | (Mb2Cas12a) | ELGDLAKFVIAQEGESSPKLAHLAHFEKFSTYFTGFHDNRKNMYSDED |
| | | KHTAIAYRLIHENLPRFIDNLQILTTIKQKHSALYDQIINELTASGLDVSL |
| | | ASHLDGYHKLLTQEGITAYNRIIGEVNGYTNKHNQICHKSERIAKLRPL |
| | | HKQILSDGMGVSFLPSKFADDSEMCQAVNEFYRHYTDVFAKVQSLFDG |
| | | FDDHQKDGIYVEHKNLNELSKQAFGDFALLGRVLDGYYVDVVNPEFN |
| | | ERFAKAKTDNAKAKLTKEKDKFIKGVHSLASLEQAIEHHTARHDDESV |
| | | QAGKLGQYFKHGLAGVDNPIQKIHNNHSTIKGFLERERPAGERALPKIK |
| | | SGKNPEMTQLRQLKELLDNALNVAHFAKLLTTKTTLDNQDGNFYGEF |
| | | GVLYDELAKIPTLYNKVRDYLSQKPFSTEKYKLNFGNPTLLNGWDLNK |
| | | EKDNFGVILQKDGCYYLALLDKAHKKVFDNAPNTGKNVYQKMVYKL |
| | | LPGPNKMLPKVFFAKSNLDYYNPSAELLDKYAKGTHKKGDNFNLKDC |
| | | HALIDFFKAGINKHPEWQHFGFKFSPTSSYRDLSDFYREVEPQGYQVKF |
| | | VDINADYIDELVEQGKLYLFQIYNKDFSPKAHGKPNLHTLYFKALFSED |
| | | NLADPIYKLNGEAQIFYRKASLDMNETTIHRAGEVLENKNPDNPKKRQ |
| | | FVYDIIKDKRYTQDKFMLHVPITMNFGVQGMTIKEFNKKVNQSIQQYD |
| | | EVNVIGIDRGERHLLYLTVINSKGEILEQRSLNDITTASANGTQVTTPYH |
| | | KILDKREIERLNARVGWGEIETIKELKSGYLSHVVHQINQLMLKYNAIV |
| | | VLEDLNFGFKRGRFKVEKQIYQNFENALIKKLNHLVLKDKADDEIGSY |
| | | KNALQLTNNFTDLKSIGKQTGFLFYVPAWNTSKIDPETGFVDLLKPRYE |
| | | NIAQSQAFFGKFDKICYNTDKGYFEFHIDYAKFTDKAKNSRQKWAICS |
| | | HGDKRYVYDKTANQNKGAAKGINVNDELKSLFARYHINDKQPNLVM |
| | | DICQNNDKEFHKSLMCLLKTLLALRYSNASSDEDFILSPVANDEGVFFN |
| | | SALADDTQPQNADANGAYHIALKGLWLLNELKNSDDLNKVKLAIDNQ |
| | | TWLNFAQNR |
| SEQ | Moraxella | MGIHGVPAALFQDFTHLYPLSKTVRFELKPIGKTLEHIHAKNFLNQDET |
| ID | bovoculi | MADMYQKVKAILDDYHRDFIADMMGEVKLTKLAEFYDVYLKFRKNP |
| NO: | AAXH_00205 | KDDGLQKQLKDLQAVLRKEIVKPIGNGGKYKAGYDRLFGAKLFKDGK |
| 33 | (Mb3Cas12a) | ELGDLAKFVIAQEGESSPKLAHLAHFEKFSTYFTGFHDNRKNMYSDED |
| | | KHTAIAYRLIHENLPRFIDNLQILATIKQKHSALYDQIINELTASGLDVSL |
| | | ASHLDGYHKLLTQEGITAYNTLLGGISGEAGSRKIQGINELINSHHNQH |
| | | CHKSERIAKLRPLHKQILSDGMGVSFLPSKFADDSEVCQAVNEFYRHY |
| | | ADVFAKVQSLFDGFDDYQKDGIYVEYKNLNELSKQAFGDFALLGRVL |
| | | DGYYVDVVNPEFNERFAKAKTDNAKAKLTKEKDKFIKGVHSLASLEQ |
| | | AIEHYTARHDDESVQAGKLGQYFKHGLAGVDNPIQKIHNNHSTIKGFL |
| | | ERERPAGERALPKIKSDKSPEIRQLKELLDNALNVAHFAKLLTTKTTLH |
| | | NQDGNFYGEFGALYDELAKIATLYNKVRDYLSQKPFSTEKYKLNFGNP |
| | | TLLNGWDLNKEKDNFGVILQKDGCYYLALLDKAHKKVFDNAPNTGKS |
| | | VYQKMIYKLLPGPNKMLPKVFFAKSNLDYYNPSAELLDKYAQGTHKK |
| | | GDNFNLKDCHALIDFFKAGINKHPEWQHFGFKFSPTSSYQDLSDFYREV |
| | | EPQGYQVKFVDINADYINELVEQGQLYLFQIYNKDFSPKAHGKPNLHT |
| | | LYFKALFSEDNLVNPIYKLNGEAEIFYRKASLDMNETTIHRAGEVLENK |
| | | NPDNPKKRQFVYDIIKDKRYTQDKFMLHVPITMNFGVQGMTIKEFNKK |
| | | VNQSIQQYDEVNVIGIDRGERHLLYLTVINSKGEILEQRSLNDITTASAN |
| | | GTQMTTPYHKILDKREIERLNARVGWGEIETIKELKSGYLSHVVHQISQ |
| | | LMLKYNAIVVLEDLNFGFKRGRFKVEKQIYQNFENALIKKLNHLVLKD |
| | | KADDEIGSYKNALQLTNNFTDLKSIGKQTGFLFYVPAWNTSKIDPETGF |
| | | VDLLKPRYENIAQSQAFFGKFDKICYNADRGYFEFHIDYAKFNDKAKN |
| | | SRQIWKICSHGDKRYVYDKTANQNKGATIGVNVNDELKSLFTRYHIND |
| | | KQPNLVMDICQNNDKEFHKSLMYLLKTLLALRYSNASSDEDFILSPVA |
| | | NDEGVFFNSALADDTQPQNADANGAYHIALKGLWLLNELKNSDDLNK |
| | | VKLAIDNQTWLNFAQNR |
| SEQ | Thiomicrospira | MGIHGVPAATKTFDSEFFNLYSLQKTVRFELKPVGETASFVEDFKNEGL |
| ID | sp. XS5 | KRVVSEDERRAVDYQKVKEIIDDYHRDFIEESLNYFPEQVSKDALEQAF |
| NO: | (TsCas12a) | HLYQKLKAAKVEEREKALKEWEALQKKLREKVVKCFSDSNKARFSRI |
| 34 | | DKKELIKEDLINWLVAQNREDDIPTVETFNNFTTYFTGFHENRKNIYSK |
| | | DDHATAISFRLIHENLPKFFDNVISFNKLKEGFPELKFDKVKEDLEVDYD |
| | | LKHAFEIEYFVNFVTQAGIDQYNYLLGGKTLEDGTKKQGMNEQINLFK |
| | | QQQTRDKARQIPKLIPLFKQILSERTESQSFIPKQFESDQELFDSLQKLHN |
| | | NCQDKFTVLQQAILGLAEADLKKVFIKTSDLNALSNTIFGNYSVFSDAL |
| | | NLYKESLKTKKAQEAFEKLPAHSIHDLIQYLEQFNSSLDAEKQQSTDTV |
| | | LNYFIKTDELYSRFIKSTSEAFTQVQPLFELEALSSKRRPPESEDEGAKG |
| | | QEGFEQIKRIKAYLDTLMEAVHFAKPLYLVKGRKMIEGLDKDQSFYEA |
| | | FEMAYQELESLIIPIYNKARSYLSRKPFKADKFKINFDNNTLLSGWDAN |
| | | KETANASILFKKDGLYYLGIMPKGKTFLFDYFVSSEDSEKLKQRRQKTA |
| | | EEALAQDGESYFEKIRYKLLPGASKMLPKVFFSNKNIGFYNPSDDILRIR |
| | | NTASHTKNGTPQKGHSKVEFNLNDCHKMIDFFKSSIQKHPEWGSFGFTF |
| | | SDTSDFEDMSAFYREVENQGYVISFDKIKETYIQSQVEQGNLYLFQIYN |
| | | KDFSPYSKGKPNLHTLYWKALFEEANLNNVVAKLNGEAEIFFRRHSIK |
| | | ASDKVVHPANQAIDNKNPHTEKTQSTFEYDLVKDKRYTQDKFFFHVPI |
| | | SLNFKAQGVSKFNDKVNGFLKGNPDVNIIGIDRGERHLLYFTVVNQKG |
| | | EILVQESLNTLMSDKGHVNDYQQKLDKKEQERDAARKSWTTVENIKE |
| | | LKEGYLSHVVHKLAHLIIKYNAIVCLEDLNFGFKRGRFKVEKQVYQKF |
| | | EKALIDKLNYLVFKEKELGEVGHYLTAYQLTAPFESFKKLGKQSGILFY |
| | | VPADYTSKIDPTTGFVNFLDLRYQSVEKAKQLLSDFNAIRFNSVQNYFE |
| | | FEIDYKKLTPKRKVGTQSKWVICTYGDVRYQNRRNQKGHWETEEVNV |
| | | TEKLKALFASDSKTTTVIDYANDDNLIDVILEQDKASFFKELLWLLKLT |
| | | MTLRHSKIKSEDDFILSPVKNEQGEFYDSRKAGEVWPKDADANGAYHI |
| | | ALKGLWNLQQINQWEKGKTLNLAIKNQDWFSFIQEKPYQE |
| SEQ | Butyrivibrio | MGIHGVPAAYYQNLTKKYPVSKTIRNELIPIGKTLENIRKNNILESDVKR |
| ID | sp. NC3005 | KQDYEHVKGIMDEYHKQLINEALDNYMLPSLNQAAEIYLKKHVDVED |
| NO: | (BsCas12a) | REEFKKTQDLLRREVTGRLKEHENYTKIGKKDILDLLEKLPSISEEDYN |
| 35 | | ALESFRNFYTYFTSYNKVRENLYSDEEKSSTVAYRLINENLPKFLDNIKS |
| | | YAFVKAAGVLADCIEEEEQDALFMVETFNMTLTQEGIDMYNYQIGKV |
| | | NSAINLYNQKNHKVEEFKKIPKMKVLYKQILSDREEVFIGEFKDDETLL |
| | | SSIGAYGNVLMTYLKSEKINIFFDALRESEGKNVYVKNDLSKTTMSNIV |
| | | FGSWSAFDELLNQEYDLANENKKKDDKYFEKRQKELKKNKSYTLEQM |
| | | SNLSKEDISPIENYIERISEDIEKICIYNGEFEKIVVNEHDSSRKLSKNIKAV |
| | | KVIKDYLDSIKELEHDIKLINGSGQELEKNLVVYVGQEEALEQLRPVDS |
| | | LYNLTRNYLTKKPFSTEKVKLNFNKSTLLNGWDKNKETDNLGILFFKD |
| | | GKYYLGIMNTTANKAFVNPPAAKTENVFKKVDYKLLPGSNKMLPKVF |
| | | FAKSNIGYYNPSTELYSNYKKGTHKKGPSFSIDDCHNLIDFFKESIKKHE |
| | | DWSKFGFEFSDTADYRDISEFYREVEKQGYKLTFTDIDESYINDLIEKNE |
| | | LYLFQIYNKDFSEYSKGKLNLHTLYFMMLFDQRNLDNVVYKLNGEAE |
| | | VFYRPASIAENELVIHKAGEGIKNKNPNRAKVKETSTFSYDIVKDKRYS |
| | | KYKFTLHIPITMNFGVDEVRRFNDVINNALRTDDNVNVIGIDRGERNLL |
| | | YVVVINSEGKILEQISLNSIINKEYDIETNYHALLDEREDDRNKARKDW |
| | | NTIENIKELKTGYLSQVVNVVAKLVLKYNAIICLEDLNFGFKRGRQKVE |
| | | KQVYQKFEKMLIEKLNYLVIDKSREQVSPEKMGGALNALQLTSKFKSF |
| | | AELGKQSGIIYYVPAYLTSKIDPTTGFVNLFYIKYENIEKAKQFFDGFDFI |
| | | RFNKKDDMFEFSFDYKSFTQKACGIRSKWIVYTNGERIIKYPNPEKNNL |
| | | FDEKVINVTDEIKGLFKQYRIPYENGEDIKEIIISKAEADFYKRLFRLLHQ |
| | | TLQMRNSTSDGTRDYIISPVKNDRGEFFCSEFSEGTMPKDADANGAYNI |
| | | ARKGLWVLEQIRQKDEGEKVNLSMTNAEWLKYAQLHLL |
| SEQ | AacCas12b | MAVKSIKVKLRLDDMPEIRAGLWKLHKEVNAGVRYYTEWLSLLRQEN |
| ID | | LYRRSPNGDGEQECDKTAEECKAELLERLRARQVENGHRGPAGSDDEL |
| NO: | | LQLARQLYELLVPQAIGAKGDAQQIARKFLSPLADKDAVGGLGIAKAG |
| 36 | | NKPRWVRMREAGEPGWEEEKEKAETRKSADRTADVLRALADFGLKPL |
| | | MRVYTDSEMSSVEWKPLRKGQAVRTWDRDMFQQAIERMMSWESWN |
| | | QRVGQEYAKLVEQKNRFEQKNFVGQEHLVHLVNQLQQDMKEASPGL |
| | | ESKEQTAHYVTGRALRGSDKVFEKWGKLAPDAPFDLYDAEIKNVQRR |
| | | NTRRFGSHDLFAKLAEPEYQALWREDASFLTRYAVYNSILRKLNHAKM |
| | | FATFTLPDATAHPIWTRFDKLGGNLHQYTFLFNEFGERRHAIRFHKLLK |
| | | VENGVAREVDDVTVPISMSEQLDNLLPRDPNEPIALYFRDYGAEQHFT |
| | | GEFGGAKIQCRRDQLAHMHRRRGARDVYLNVSVRVQSQSEARGERRP |
| | | PYAAVFRLVGDNHRAFVHFDKLSDYLAEHPDDGKLGSEGLLSGLRVM |
| | | SVDLGLRTSASISVFRVARKDELKPNSKGRVPFFFPIKGNDNLVAVHER |
| | | SQLLKLPGETESKDLRAIREERQRTLRQLRTQLAYLRLLVRCGSEDVGR |
| | | RERSWAKLIEQPVDAANHMTPDWREAFENELQKLKSLHGICSDKEWM |
| | | DAVYESVRRVWRHMGKQVRDWRKDVRSGERPKIRGYAKDVVGGNSI |
| | | EQIEYLERQYKFLKSWSFFGKVSGQVIRAEKGSRFAITLREHIDHAKED |
| | | RLKKLADRIIMEALGYVYALDERGKGKWVAKYPPCQLILLEELSEYQF |
| | | NNDRPPSENNQLMQWSHRGVFQELINQAQVHDLLVGTMYAAFSSRFD |
| | | ARTGAPGIRCRRVPARCTQEHNPEPFPWWLNKFVVEHTLDACPLRADD |
| | | LIPTGEGEIFVSPFSAEEGDFHQIHADLNAAQNLQQRLWSDFDISQIRLR |
| | | CDWGEVDGELVLIPRLTGKRTADSYSNKVFYTNTGVTYYERERGKKR |
| | | RKVFAQEKLSEEEAELLVEADEAREKSVVLMRDPSGIINRGNWTRQKE |
| | | FWSMVNQRIEGYLVKQIRSRVPLQDSACENTGDI |
| SEQ | Cas12 | MKKIDNFVGCYPVSKTLRFKAIPIGKTQENIEKKRLVEEDEVRAK |
| ID | Variant | DYKAVKKLIDRYHREFIEGVLDNVKLDGLEEYYMLFNKSDREES |
| NO: | | DNKKIEIMEERFRRVISKSFKNNEEYKKIFSKKIIEEILPNYIKDEEE |
| 37 | | KELVKGFKGFYTAFVGYAQNRENMYSDEKKSTAISYRIVNENMP |
| | | RFITNIKVFEKAKSILDVDKINEINEYILNNDYYVDDFFNIDFFNYV |
| | | LNQKGIDIYNAIIGGIVTGDGRKIQGLNECINLYNQENKKIRLPQF |
| | | KPLYKQILSESESMSFYIDEIESDDMLIDMLKESLQIDSTINNAIDD |
| | | LKVLFNNIFDYDLSGIFINNGLPITTISNDVYGQWSTISDGWNERY |
| | | DVLSNAKDKESEKYFEKRRKEYKKVKSFSISDLQELGGKDLSICK |
| | | KINEIISEMIDDYKSKIEEIQYLFDIKELEKPLVTDLNKIELIKNSLD |
| | | GLKRIERYVIPFLGTGKEQNRDEVFYGYFIKCIDAIKEIDGVYNKT |
| | | RNYLTKKPYSKDKFKLYFENPQLMGGWDRNKESDYRSTLLRKN |
| | | GKYYVAIIDKSSSNCMMNIEEDENDNYEKINYKLLPGPNKMLPK |
| | | VFFSKKNREYFAPSKEIERIYSTGTFKKDTNFVKKDCENLITFYKD |
| | | SLDRHEDWSKSFDFSFKESSAYRDISEFYRDVEKQGYRVSFDLLS |
| | | SNAVNTLVEEGKLYLFQLYNKDFSEKSHGIPNLHTMYFRSLFDD |
| | | NNKGNIRLNGGAEMFMRRASLNKQDVTVHKANQPIKNKNLLNP |
| | | KKTTTLPYDVYKDKRFTEDQYEVHIPITMNKVPNNPYKINHMVR |
| | | EQLVKDDNPYVIGIDRGERNLIYVVVVDGQGHIVEQLSLNEIINE |
| | | NNGISIRTDYHTLLDAKERERDESRKQWKQIENIKELKEGYISQV |
| | | VHKICELVEKYDAVIALEDLNSGFKNSRVKVEKQVYQKFEKMLI |
| | | TKLNYMVDKKKDYNKPGGVLNGYQLTTQFESFSKMGTQNGIMF |
| | | YIPAWLTSKMDPTTGFVDLLKPKYKNKADAQKFFSQFDSIRYDN |
| | | QEDAFVFKVNYTKFPRTDADYNKEWEIYTNGERIRVFRNPKKNN |
| | | EYDYETVNVSERMKELFDSYDLLYDKGELKETICEMEESKFFEEL |
| | | IKLFRLTLQMRNSISGRTDVDYLISPVKNSNGYFYNSNDYKKEGA |
| | | KYPKDADANGAYNIARKVLWAIEQFKMADEDKLDKTKISIKNQ |
| | | EWLEYAQTHCE |

Alternatively, the Type V CRISPR/Cas enzyme is a programmable Cas14 nuclease. A Cas14 protein of the present disclosure includes 3 partial RuvC domains (RuvC-I, RuvC-II, and RuvC-III, also referred to herein as subdomains) that are not contiguous with respect to the primary amino acid sequence of the Cas14 protein, but form a RuvC domain once the protein is produced and folds. A naturally occurring Cas14 protein functions as an endonuclease that catalyzes cleavage at a specific sequence in a target nucleic acid. A programmable Cas14 nuclease can be a Cas14a protein, a Cas14b protein, a Cas14c protein, a Cas14d protein, a Cas14e protein, a Cas 14f protein, a Cas14g protein, a Cas14h protein, or a Cas14u protein. In some cases, a suitable Cas14 protein comprises an amino acid sequence having at least 80%, at least 85%, at least 90%, at least 95%, at least 98%, at least 99%, or 100%, amino acid sequence identity to any one of SEQ ID NO: 38-SEQ ID NO: 129.

TABLE 2
Cas14 Protein Sequences

| SEQ | |
|---|---|
| ID | |
| NO | Sequence |
| SEQ | MEVQKTVMKTLSLRILRPLYSQEIEKEIKEEKERRKQAGGTGELDGGFYKKLEKKHSE |
| ID | MFSFDRLNLLLNQLQREIAKVYNHAISELYIATIAQGNKSNKHYISSIVYNRAYGYFYN |
| NO: | AYIALGICSKVEANFRSNELLTQQSALPTAKSDNFPIVLHKQKGAEGEDGGFRISTEGS |
| 38 | DLIFEIPIPFYEYNGENRKEPYKWVKKGGQKPVLKLILSTFRRQRNKGWAKDEGTDAE |
| | IRKVTEGKYQVSQIEINRGKKLGEHQKWFANFSIEQPIYERKPNRSIVGGLDVGIRSPLV |
| | CAINNSFSRYSVDSNDVFKFSKQVFAFRRRLLSKNSLKRKGHGAAHKLEPITEMTEKN |
| | DKFRKKIIERWAKEVTNFFVKNQVGIVQIEDLSTMKDREDHFFNQYLRGFWPYYQMQ |
| | TLIENKLKEYGIEVKRVQAKYTSQLCSNPNCRYWNNYFNFEYRKVNKFPKFKCEKCN |
| | LEISADYNAARNLSTPDIEKFVAKATKGINLPEK |
| SEQ | MEEAKTVSKTLSLRILRPLYSAEIEKEIKEEKERRKQGGKSGELDSGFYKKLEKKHTQ |
| ID | MFGWDKLNLMLSQLQRQIARVFNQSISELYIETVIQGKKSNKHYTSKIVYNRAYSVFY |
| NO: | NAYLALGITSKVEANFRSTELLMQKSSLPTAKSDNFPILLHKQKGVEGEEGGFKISADG |
| 39 | NDLIFEIPIPFYEYDSANKKEPFKWIKKGGQKPTIKLILSTFRRQRNKGWAKDEGTDAEI |
| | RKVIEGKYQVSHIEINRGKKLGDHQKWFVNFTIEQPIYERKLDKNIIGGIDVGIKSPLVC |
| | AVNNSFARYSVDSNDVLKFSKQAFAFRRRLLSKNSLKRSGHGSKNKLDPITRMTEKN |
| | DRFRKKIIERWAKEVTNFFIKNQVGTVQIEDLSTMKDRQDNFFNQYLRGFWPYYQMQ |
| | NLIENKLKEYGIETKRIKARYTSQLCSNPSCRHWNSYFSFDHRKTNNFPKFKCEKCALE |
| | ISADYNAARNISTPDIEKFVAKATKGINLPDKNENVILE |
| SEQ | MAKNTITKTLKLRIVRPYNSAEVEKIVADEKNNREKIALEKNKDKVKEACSKHLKVA |
| ID | AYCTTQVERNACLFCKARKLDDKFYQKLRGQFPDAVFWQEISEIFRQLQKQAAEIYN |
| NO: | QSLIELYYEIFIKGKGIANASSVEHYLSDVCYTRAAELFKNAAIASGLRSKIKSNFRLKE |
| 40 | LKNMKSGLPTTKSDNFPIPLVKQKGGQYTGFEISNHNSDFIIKIPFGRWQVKKEIDKYR |
| | PWEKFDFEQVQKSPKPISLLLSTQRRKRNKGWSKDEGTEAEIKKVMNGDYQTSYIEVK |
| | RGSKIGEKSAWMLNLSIDVPKIDKGVDPSIIGGIDVGVKSPLVCAINNAFSRYSISDNDL |
| | FHFNKKMFARRRILLKKNRHKRAGHGAKNKLKPITILTEKSERFRKKLIERWACEIADF |
| | FIKNKVGTVQMENLESMKRKEDSYFNIRLRGFWPYAEMQNKIEFKLKQYGIEIRKVAP |
| | NNTSKTCSKCGHLNNYFNFEYRKKNKFPHFKCEKCNFKENADYNAALNISNPKLKST |
| | KEEP |
| SEQ | MERQKVPQIRKIVRVVPLRILRPKYSDVIENALKKFKEKGDDTNTNDFWRAIRDRDTE |
| ID | FFRKELNFSEDEINQLERDTLFRVGLDNRVLFSYFDFLQEKLMKDYNKIISKLFINRQSK |
| NO: | SSFENDLTDEEVEELIEKDVTPFYGAYIGKGIKSVIKSNLGGKFIKSVKIDRETKKVTKL |
| 41 | TAINIGLMGLPVAKSDTFPIKIIKTNPDYITFQKSTKENLQKIEDYETGIEYGDLLVQITIP |
| | WFKNENKDFSLIKTKEAIEYYKLNGVGKKDLLNINLVLTTYHIRKKKSWQIDGSSQSL |
| | VREMANGELEEKWKSFFDTFIKKYGDEGKSALVKRRVNKKSRAKGEKGRELNLDERI |
| | KRLYDSIKAKSFPSEINLIPENYKWKLHFSIEIPPMVNDIDSNLYGGIDFGEQNIATLCVK |
| | NIEKDDYDFLTIYGNDLLKHAQASYARRRIMRVQDEYKARGHGKSRKTKAQEDYSER |
| | MQKLRQKITERLVKQISDFFLWRNKFHMAVCSLRYEDLNTLYKGESVKAKRMRQFIN |
| | KQQLFNGIERKLKDYNSEIYVNSRYPHYTSRLCSKCGKLNLYFDFLKFRTKNIIIRKNP |
| | DGSEIKYMPFFICEFCGWKQAGDKNASANIADKDYQDKLNKEKEFCNIRKPKSKKEDI |
| | GEENEEERDYSRRFNRNSFIYNSLKKDNKLNQEKLFDEWKNQLKRKIDGRNKFEPKE |
| | YKDRFSYLFAYYQEIIKNESES |
| SEQ | MVPTELITKTLQLRVIRPLYFEEIEKELAELKEQKEKEFEETNSLLLESKKIDAKSLKKL |
| ID | KRKARSSAAVEFWKIAKEKYPDILTKPEMEFIFSEMQKMMARFYNKSMTNIFIEMNND |
| NO: | EKVNPLSLISKASTEANQVIKCSSISSGLNRKIAGSINKTKFKQVRDGLISLPTARTETFPI |
| 42 | SFYKSTANKDEIPISKINLPSEEEADLTITLPFPFFEIKKEKKGQKAYSYFNIIEKSGRSNN |
| | KIDLLLSTHRRQRRKGWKEEGGTSAEIRRLMEGEFDKEWEIYLGEAEKSEKAKNDLIK |
| | NMTRGKLSKDIKEQLEDIQVKYFSDNNVESWNDLSKEQKQELSKLRKKKVEELKDW |
| | KHVKEILKTRAKIGWVELKRGKRQRDRNKWFVNITITRPPFINKELDDTKFGGIDLGV |
| | KVPFVCAVHGSPARLIIKENEILQFNKMVSARNRQITKDSEQRKGRGKKNKFIKKEIFN |
| | ERNELFRKKIIERWANQIVKFFEDQKCATVQIENLESFDRTSYK |
| SEQ | MKSDTKDKKIIIHQTKTLSLRIVKPQSIPMEEFTDLVRYHQMIIFPVYNNGAIDLYKKLF |
| ID | KAKIQKGNEARAIKYFMNKIVYAPIANTVKNSYIALGYSTKMQSSFSGKRLWDLRFGE |
| NO: | ATPPTIKADFPLPFYNQSGFKVSSENGEFIIGIPFGQYTKKTVSDIEKKTSFAWDKFTLED |
| 43 | TTKKTLIELLLSTKTRKMNEGWKNNEGTEAEIKRVMDGTYQVTSLEILQRDDSWFVN |
| | FNIAYDSLKKQPDRDKIAGIHMGITRPLTAVIYNNKYRALSIYPNTVMHLTQKQLARIK |
| | EQRTNSKYATGGHGRNAKVTGTDTLSEAYRQRRKKIIEDWIASIVKFAINNEIGTIYLE |
| | DISNTNSFFAAREQKLIYLEDISNTNSFLSTYKYPISAISDTLQHKLEEKAIQVIRKKAYY |
| | VNQICSLCGHYNKGFTYQFRRKNKFPKMKCQGCLEATSTEFNAAANVANPDYEKLLI |
| | KHGLLQLKK |
| SEQ | MSTITRQVRLSPTPEQSRLLMAHCQQYISTVNVLVAAFDSEVLTGKVSTKDFRAALPS |
| ID | AVKNQALRDAQSVFKRSVELGCLPVLKKPHCQWNNQNWRVEGDQLILPICKDGKTQ |
| NO: | QERFRCAAVALEGKAGILRIKKKRGKWIADLTVTQEDAPESSGSAIMGVDLGIKVPAV |
| 44 | AHIGGKGTRFFGNGRSQRSMRRRFYARRKTLQKAKKLRAVRKSKGKEARWMKTINH |
| | QLSRQIVNHAHALGVGTIKIEALQGIRKGTTRKSRGAAARKNNRMTNTWSFSQLTLFI |
| | TYKAQRQGITVEQVDPAYTSQDCPACRARNGAQDRTYVCSECGWRGHRDTVGAINIS |
| | RRAGLSGHRRGATGA |
| SEQ | MIAQKTIKIKLNPTKEQIIKLNSIIEEYIKVSNFTAKKIAEIQESFTDSGLTQGTCSECGKE |
| ID | KTYRKYHLLKKDNKLFCITCYKRKYSQFTLQKVEFQNKTGLRNVAKLPKTYYTNAIR |
| NO: | FASDTFSGFDEIIKKKQNRLNSIQNRLNFWKELLYNPSNRNEIKIKVVKYAPKTDTREH |
| 45 | PHYYSEAEIKGRIKRLEKQLKKFKMPKYPEFTSETISLQRELYSWKNPDELKISSITDKN |
| | ESMNYYGKEYLKRYIDLINSQTPQILLEKENNSFYLCFPITKNIEMPKIDDTFEPVGIDW |
| | GITRNIAVVSILDSKTKKPKFVKFYSAGYILGKRKHYKSLRKHFGQKKRQDKINKLGT |
| | KEDRFIDSNIHKLAFLIVKEIRNHSNKPIILMENITDNREEAEKSMRQNILLHSVKSRLQ |
| | NYIAYKALWNNIPTNLVKPEHTSQICNRCGHQDRENRPKGSKLFKCVKCNYMSNADF |
| | NASINIARKFYIGEYEPFYKDNEKMKSGVNSISM |
| SEQ | LKLSEQENITTGVKFKLKLDKETSEGLNDYFDEYGKAINFAIKVIQKELAEDRFAGKVR |
| ID | LDENKKPLLNEDGKKIWDFPNEFCSCGKQVNRYVNGKSLCQECYKNKFTEYGIRKRM |
| NO: | YSAKGRKAEQDINIKNSTNKISKTHFNYAIREAFILDKSIKKQRKERFRRLREMKKKLQ |
| 46 | EFIEIRDGNKILCPKIEKQRVERYIHPSWINKEKKLEDFRGYSMSNVLGKIKILDRNIKRE |
| | EKSLKEKGQINFKARRLMLDKSVKFLNDNKISFTISKNLPKEYELDLPEKEKRLNWLK |
| | EKIKIIKNQKPKYAYLLRKDDNFYLQYTLETEFNLKEDYSGIVGIDRGVSHIAVYTFVH |
| | NNGKNERPLFLNSSEILRLKNLQKERDRFLRRKHNKKRKKSNMRNIEKKIQLILHNYS |
| | KQIVDFAKNKNAFIVFEKLEKPKKNRSKMSKKSQYKLSQFTFKKLSDLVDYKAKREGI |
| | KVLYISPEYTSKECSHCGEKVNTQRPFNGNSSLFKCNKCGVELNADYNASINIAKKGL |
| | NILNSTN |
| SEQ | MEESIITGVKFKLRIDKETTKKLNEYFDEYGKAINFAVKIIQKELADDRFAGKAKLDQN |
| ID | KNPILDENGKKIYEFPDEFCSCGKQVNKYVNNKPFCQECYKIRFTENGIRKRMYSAKG |
| NO: | RKAEHKINILNSTNKISKTHFNYAIREAFILDKSIKKQRKKRNERLRESKKRLQQFIDMR |
| 47 | DGKREICPTIKGQKVDRFIHPSWITKDKKLEDFRGYTLSIINSKIKILDRNIKREEKSLKE |
| | KGQIIFKAKRLMLDKSIRFVGDRKVLFTISKTLPKEYELDLPSKEKRLNWLKEKIEIIKN |
| | QKPKYAYLLRKNIESEKKPNYEYYLQYTLEIKPELKDFYDGAIGIDRGINHIAVCTFISN |
| | DGKVTPPKFFSSGEILRLKNLQKERDRFLLRKHNKNRKKGNMRVIENKINLILHRYSK |
| | QIVDMAKKLNASIVFEELGRIGKSRTKMKKSQRYKLSLFIFKKLSDLVDYKSRREGIRV |
| | TYVPPEYTSKECSHCGEKVNTQRPFNGNYSLFKCNKCGIQLNSDYNASINIAKKGLKIP |
| | NST |
| SEQ | LWTIVIGDFIEMPKQDLVTTGIKFKLDVDKETRKKLDDYFDEYGKAINFAVKIIQKNLK |
| ID | EDRFAGKIALGEDKKPLLDKDGKKIYNYPNESCSCGNQVRRYVNAKPFCVDCYKLKF |
| NO: | TENGIRKRMYSARGRKADSDINIKNSTNKISKTHFNYAIREGFILDKSLKKQRSKRIKKL |
| 48 | LELKRKLQEFIDIRQGQMVLCPKIKNQRVDKFIHPSWLKRDKKLEEFRGYSLSVVEGKI |
| | KIFNRNILREEDSLRQRGHVNFKANRIMLDKSVRFLDGGKVNFNLNKGLPKEYLLDLP |
| | KKENKLSWLNEKISLIKLQKPKYAYLLRREGSFFIQYTIENVPKTFSDYLGAIGIDRGIS |
| | HIAVCTFVSKNGVNKAPVFFSSGEILKLKSLQKQRDLFLRGKHNKIRKKSNMRNIDNKI |
| | NLILHKYSRNIVNLAKSEKAFIVFEKLEKIKKSRFKMSKSLQYKLSQFTFKKLSDLVEY |
| | KAKIEGIKVDYVPPEYTSKECSHCGEKVDTQRPFNGNSSLFKCNKCRVQLNADYNASI |
| | NIAKKSLNISN |
| SEQ | MSKTTISVKLKIIDLSSEKKEFLDNYFNEYAKATTFCQLRIRRLLRNTHWLGKKEKSSK |
| ID | KWIFESGICDLCGENKELVNEDRNSGEPAKICKRCYNGRYGNQMIRKLFVSTKKREVQ |
| NO: | ENMDIRRVAKLNNTHYHRIPEEAFDMIKAADTAEKRRKKNVEYDKKRQMEFIEMFND |
| 49 | EKKRAARPKKPNERETRYVHISKLESPSKGYTLNGIKRKIDGMGKKIERAEKGLSRKKI |
| | FGYQGNRIKLDSNWVRFDLAESEITIPSLFKEMKLRITGPTNVHSKSGQIYFAEWFERIN |
| | KQPNNYCYLIRKTSSNGKYEYYLQYTYEAEVEANKEYAGCLGVDIGCSKLAAAVYY |
| | DSKNKKAQKPIEIFTNPIKKIKMRREKLIKLLSRVKVRHRRRKLMQLSKTEPIIDYTCHK |
| | TARKIVEMANTAKAFISMENLETGIKQKQQARETKKQKFYRNMFLFRKLSKLIEYKAL |
| | LKGIKIVYVKPDYTSQTCSSCGADKEKTERPSQAIFRCLNPTCRYYQRDINADFNAAV |
| | NIAKKALNNTEVVTTLL |
| SEQ | MARAKNQPYQKLTTTTGIKFKLDLSEEEGKRFDEYFSEYAKAVNFCAKVIYQLRKNL |
| ID | KFAGKKELAAKEWKFEISNCDFCNKQKEIYYKNIANGQKVCKGCHRTNFSDNAIRKK |
| NO: | MIPVKGRKVESKFNIHNTTKKISGTHRHWAFEDAADIIESMDKQRKEKQKRLRREKRK |
| 50 | LSYFFELFGDPAKRYELPKVGKQRVPRYLHKIIDKDSLTKKRGYSLSYIKNKIKISERNI |
| | ERDEKSLRKASPIAFGARKIKMSKLDPKRAFDLENNVFKIPGKVIKGQYKFFGTNVAN |
| | EHGKKFYKDRISKILAGKPKYFYLLRKKVAESDGNPIFEYYVQWSIDTETPAITSYDNI |
| | LGIDAGITNLATTVLIPKNLSAEHCSHCGNNHVKPIFTKFFSGKELKAIKIKSRKQKYFL |
| | RGKHNKLVKIKRIRPIEQKVDGYCHVVSKQIVEMAKERNSCIALEKLEKPKKSKFRQR |
| | RREKYAVSMFVFKKLATFIKYKAAREGIEIIPVEPEGTSYTCSHCKNAQNNQRPYFKPN |
| | SKKSWTSMFKCGKCGIELNSDYNAAFNIAQKALNMTSA |
| SEQ | MDEKHFFCSYCNKELKISKNLINKISKGSIREDEAVSKAISIHNKKEHSLILGIKFKLFIE |
| ID | NKLDKKKLNEYFDNYSKAVTFAARIFDKIRSPYKFIGLKDKNTKKWTFPKAKCVFCLE |
| NO: | EKEVAYANEKDNSKICTECYLKEFGENGIRKKIYSTRGRKVEPKYNIFNSTKELSSTHY |
| 51 | NYAIRDAFQLLDALKKQRQKKLKSIFNQKLRLKEFEDIFSDPQKRIELSLKPHQREKRY |
| | IHLSKSGQESINRGYTLRFVRGKIKSLTRNIEREEKSLRKKTPIHFKGNRLMIFPAGIKFD |
| | FASNKVKISISKNLPNEFNFSGTNVKNEHGKSFFKSRIELIKTQKPKYAYVLRKIKREYS |
| | KLRNYEIEKIRLENPNADLCDFYLQYTIETESRNNEEINGIIGIDRGITNLACLVLLKKGD |
| | KKPSGVKFYKGNKILGMKIAYRKHLYLLKGKRNKLRKQRQIRAIEPKINLILHQISKDI |
| | VKIAKEKNFAIALEQLEKPKKARFAQRKKEKYKLALFTFKNLSTLIEYKSKREGIPVIY |
| | VPPEKTSQMCSHCAINGDEHVDTQRPYKKPNAQKPSYSLFKCNKCGIELNADYNAAF |
| | NIAQKGLKTLMLNHSH |
| SEQ | MLQTLLVKLDPSKEQYKMLYETMERFNEACNQIAETVFAIHSANKIEVQKTVYYPIRE |
| ID | KFGLSAQLTILAIRKVCEAYKRDKSIKPEFRLDGALVYDQRVLSWKGLDKVSLVTLQG |
| NO: | RQIIPIKFGDYQKARMDRIRGQADLILVKGVFYLCVVVEVSEESPYDPKGVLGVDLGIK |
| 52 | NLAVDSDGEVHSGEQTTNTRERLDSLKARLQSKGTKSAKRHLKKLSGRMAKFSKDV |
| | NHCISKKLVAKAKGTLMSIALEDLQGIRDRVTVRKAQRRNLHTWNFGLLRMFVDYK |
| | AKIAGVPLVFVDPRNTSRTCPSCGHVAKANRPTRDEFRCVSCGFAGAADHIAAMNIAF |
| | RAEVSQPIVTRFFVQSQAPSFRVG |
| SEQ | MDEEPDSAEPNLAPISVKLKLVKLDGEKLAALNDYFNEYAKAVNFCELKMQKIRKNL |
| ID | VNIRGTYLKEKKAWINQTGECCICKKIDELRCEDKNPDINGKICKKCYNGRYGNQMIR |
| NO: | KLFVSTNKRAVPKSLDIRKVARLHNTHYHRIPPEAADIIKAIETAERKRRNRILFDERRY |
| 53 | NELKDALENEEKRVARPKKPKEREVRYVPISKKDTPSKGYTMNALVRKVSGMAKKIE |
| | RAKRNLNKRKKIEYLGRRILLDKNWVRFDFDKSEISIPTMKEFFGEMRFEITGPSNVMS |
| | PNGREYFTKWFDRIKAQPDNYCYLLRKESEDETDFYLQYTWRPDAHPKKDYTGCLGI |
| | DIGGSKLASAVYFDADKNRAKQPIQIFSNPIGKWKTKRQKVIKVLSKAAVRHKTKKLE |
| | SLRNIEPRIDVHCHRIARKIVGMALAANAFISMENLEGGIREKQKAKETKKQKFSRNM |
| | FVFRKLSKLIEYKALMEGVKVVYIVPDYTSQLCSSCGTNNTKRPKQAIFMCQNTECRY |
| | FGKNINADFNAAINIAKKALNRKDIVRELS |
| SEQ | MEKNNSEQTSITTGIKFKLKLDKETKEKLNNYFDEYGKAINFAVRIIQMQLNDDRLAG |
| ID | KYKRDEKGKPILGEDGKKILEIPNDFCSCGNQVNHYVNGVSFCQECYKKRFSENGIRK |
| NO: | RMYSAKGRKAEQDINIKNSTNKISKTHFNYAIREAFNLDKSIKKQREKRFKKLKDMKR |
| 54 | KLQEFLEIRDGKRVICPKIEKQKVERYIHPSWINKEKKLEEFRGYSLSIVNSKIKSFDRNI |
| | QREEKSLKEKGQINFKAQRLMLDKSVKFLKDNKVSFTISKELPKTFELDLPKKEKKLN |
| | WLNEKLEIIKNQKPKYAYLLRKENNIFLQYTLDSIPEIHSEYSGAVGIDRGVSHIAVYTF |
| | LDKDGKNERPFFLSSSGILRLKNLQKERDKFLRKKHNKIRKKGNMRNIEQKINLILHEY |
| | SKQIVNFAKDKNAFIVFELLEKPKKSRERMSKKIQYKLSQFTFKKLSDLVDYKAKREGI |
| | KVIYVEPAYTSKDCSHCGERVNTQRPFNGNFSLFKCNKCGIVLNSDYNASLNIARKGL |
| | NISAN |
| SEQ | MAEEKFFFCEKCNKDIKIPKNYINKQGAEEKARAKHEHRVHALILGIKFKIYPKKEDIS |
| ID | KLNDYFDEYAKAVTFTAKIVDKLKAPFLFAGKRDKDTSKKKWVFPVDKCSFCKEKTE |
| NO: | INYRTKQGKNICNSCYLTEFGEQGLLEKIYATKGRKVSSSFNLFNSTKKLTGTHNNYV |
| 55 | VKESLQLLDALKKQRSKRLKKLSNTRRKLKQFEEMFEKEDKRFQLPLKEKQRELRFIH |
| | VSQKDRATEFKGYTMNKIKSKIKVLRRNIEREQRSLNRKSPVFFRGTRIRLSPSVQFDD |
| | KDNKIKLTLSKELPKEYSFSGLNVANEHGRKFFAEKLKLIKENKSKYAYLLRRQVNKN |
| | NKKPIYDYYLQYTVEFLPNIITNYNGILGIDRGINTLACIVLLENKKEKPSFVKFFSGKGI |
| | LNLKNKRRKQLYFLKGVHNKYRKQQKIRPIEPRIDQILHDISKQIIDLAKEKRVAISLEQ |
| | LEKPQKPKFRQSRKAKYKLSQFNFKTLSNYIDYKAKKEGIRVIYIAPEMTSQNCSRCA |
| | MKNDLHVNTQRPYKNTSSLFKCNKCGVELNADYNAAFNIAQKGLKILNS |
| SEQ | MISLKLKLLPDEEQKKLLDEMFWKWASICTRVGFGRADKEDLKPPKDAEGVWFSLTQ |
| ID | LNQANTDINDLREAMKHQKHRLEYEKNRLEAQRDDTQDALKNPDRREISTKRKDLFR |
| NO: | PKASVEKGFLKLKYHQERYWVRRLKEINKLIERKTKTLIKIEKGRIKFKATRITLHQGS |
| 56 | FKIRFGDKPAFLIKALSGKNQIDAPFVVVPEQPICGSVVNSKKYLDEITTNFLAYSVNA |
| | MLFGLSRSEEMLLKAKRPEKIKKKEEKLAKKQSAFENKKKELQKLLGRELTQQEEAII |
| | EETRNQFFQDFEVKITKQYSELLSKIANELKQKNDFLKVNKYPILLRKPLKKAKSKKIN |
| | NLSPSEWKYYLQFGVKPLLKQKSRRKSRNVLGIDRGLKHLLAVTVLEPDKKTFVWNK |
| | LYPNPITGWKWRRRKLLRSLKRLKRRIKSQKHETIHENQTRKKLKSLQGRIDDLLHNIS |
| | RKIVETAKEYDAVIVVEDLQSMRQHGRSKGNRLKTLNYALSLFDYANVMQLIKYKA |
| | GIEGIQIYDVKPAGTSQNCAYCLLAQRDSHEYKRSQENSKIGVCLNPNCQNHKKQIDA |
| | DLNAARVIASCYALKINDSQPFGTRKRFKKRTTN |
| SEQ | METLSLKLKLNPSKEQLLVLDKMFWKWASICTRLGLKKAEMSDLEPPKDAEGVWFS |
| ID | KTQLNQANTDVNDLRKAMQHQGKRIEYELDKVENRRNEIQEMLEKPDRRDISPNRKD |
| NO: | LFRPKAAVEKGYLKLKYHKLGYWSKELKTANKLIERKRKTLAKIDAGKMKFKPTRIS |
| 57 | LHTNSFRIKFGEEPKIALSTTSKHEKIELPLITSLQRPLKTSCAKKSKTYLDAAILNFLAY |
| | STNAALFGLSRSEEMLLKAKKPEKIEKRDRKLATKRESFDKKLKTLEKLLERKLSEKE |
| | KSVFKRKQTEFFDKFCITLDETYVEALHRIAEELVSKNKYLEIKKYPVLLRKPESRLRS |
| | KKLKNLKPEDWTYYIQFGFQPLLDTPKPIKTKTVLGIDRGVRHLLAVSIFDPRTKTFTF |
| | NRLYSNPIVDWKWRRRKLLRSIKRLKRRLKSEKHVHLHENQFKAKLRSLEGRIEDHFH |
| | NLSKEIVDLAKENNSVIVVENLGGMRQHGRGRGKWLKALNYALSHFDYAKVMQLIK |
| | YKAELAGVFVYDVAPAGTSINCAYCLLNDKDASNYTRGKVINGKKNTKIGECKTCKK |
| | EFDADLNAARVIALCYEKRLNDPQPFGTRKQFKPKKP |
| SEQ | MKALKLQLIPTRKQYKILDEMFWKWASLANRVSQKGESKETLAPKKDIQKIQFNATQ |
| ID | LNQIEKDIKDLRGAMKEQQKQKERLLLQIQERRSTISEMLNDDNNKERDPHRPLNFRP |
| NO: | KGWRKFHTSKHWVGELSKILRQEDRVKKTIERIVAGKISFKPKRIGIWSSNYKINFFKR |
| 58 | KISINPLNSKGFELTLMTEPTQDLIGKNGGKSVLNNKRYLDDSIKSLLMFALHSRFFGL |
| | NNTDTYLLGGKINPSLVKYYKKNQDMGEFGREIVEKFERKLKQEINEQQKKIIMSQIK |
| | EQYSNRDSAFNKDYLGLINEFSEVFNQRKSERAEYLLDSFEDKIKQIKQEIGESLNISDW |
| | DFLIDEAKKAYGYEEGFTEYVYSKRYLEILNKIVKAVLITDIYFDLRKYPILLRKPLDKI |
| | KKISNLKPDEWSYYIQFGYDSINPVQLMSTDKFLGIDRGLTHLLAYSVFDKEKKEFIIN |
| | QLEPNPIMGWKWKLRKVKRSLQHLERRIRAQKMVKLPENQMKKKLKSIEPKIEVHYH |
| | NISRKIVNLAKDYNASIVVESLEGGGLKQHGRKKNARNRSLNYALSLFDYGKIASLIK |
| | YKADLEGVPMYEVLPAYTSQQCAKCVLEKGSFVDPEIIGYVEDIGIKGSLLDSLFEGTE |
| | LSSIQVLKKIKNKIELSARDNHNKEINLILKYNFKGLVIVRGQDKEEIAEHPIKEINGKFA |
| | ILDFVYKRGKEKVGKKGNQKVRYTGNKKVGYCSKHGQVDADLNASRVIALCKYLDI |
| | NDPILFGEQRKSFK |
| SEQ | MVTRAIKLKLDPTKNQYKLLNEMFWKWASLANRFSQKGASKETLAPKDGTQKIQFN |
| ID | ATQLNQIKKDVDDLRGAMEKQGKQKERLLIQIQERLLTISEILRDDSKKEKDPHRPQNF |
| NO: | RPFGWRRFHTSAYWSSEASKLTRQVDRVRRTIERIKAGKINFKPKRIGLWSSTYKINFL |
| 59 | KKKINISPLKSKSFELDLITEPQQKIIGKEGGKSVANSKKYLDDSIKSLLIFAIKSRLFGLN |
| | NKDKPLFENIITPNLVRYHKKGQEQENFKKEVIKKFENKLKKEISQKQKEIIFSQIERQY |
| | ENRDATFSEDYLRAISEFSEIFNQRKKERAKELLNSFNEKIRQLKKEVNGNISEEDLKIL |
| | EVEAEKAYNYENGFIEWEYSEQFLGVLEKIARAVLISDNYFDLKKYPILIRKPTNKSKK |
| | ITNLKPEEWDYYIQFGYGLINSPMKIETKNFMGIDRGLTHLLAYSIFDRDSEKFTINQLE |
| | LNPIKGWKWKLRKVKRSLQHLERRMRAQKGVKLPENQMKKRLKSIEPKIESYYHNLS |
| | RKIVNLAKANNASIVVESLEGGGLKQHGRKKNSRHRALNYALSLFDYGKIASLIKYKS |
| | DLEGVPMYEVLPAYTSQQCAKCVLKKGSFVEPEIIGYIEEIGFKENLLTLLFEDTGLSSV |
| | QVLKKSKNKMTLSARDKEGKMVDLVLKYNFKGLVISQEKKKEEIVEFPIKEIDGKFAV |
| | LDSAYKRGKERISKKGNQKLVYTGNKKVGYCSVHGQVDADLNASRVIALCKYLGINE |
| | PIVFGEQRKSFK |
| SEQ | LDLITEPIQPHKSSSLRSKEFLEYQISDFLNFSLHSLFFGLASNEGPLVDFKIYDKIVIPKP |
| ID | EERFPKKESEEGKKLDSFDKRVEEYYSDKLEKKIERKLNTEEKNVIDREKTRIWGEVN |
| NO: | KLEEIRSIIDEINEIKKQKHISEKSKLLGEKWKKVNNIQETLLSQEYVSLISNLSDELTNK |
| 60 | KKELLAKKYSKFDDKIKKIKEDYGLEFDENTIKKEGEKAFLNPDKFSKYQFSSSYLKLI |
| | GEIARSLITYKGFLDLNKYPIIFRKPINKVKKIHNLEPDEWKYYIQFGYEQINNPKLETE |
| | NILGIDRGLTHILAYSVFEPRSSKFILNKLEPNPIEGWKWKLRKLRRSIQNLERRWRAQ |
| | DNVKLPENQMKKNLRSIEDKVENLYHNLSRKIVDLAKEKNACIVFEKLEGQGMKQHG |
| | RKKSDRLRGLNYKLSLFDYGKIAKLIKYKAEIEGIPIYRIDSAYTSQNCAKCVLESRRFA |
| | QPEEISCLDDFKEGDNLDKRILEGTGLVEAKIYKKLLKEKKEDFEIEEDIAMFDTKKVI |
| | KENKEKTVILDYVYTRRKEIIGTNHKKNIKGIAKYTGNTKIGYCMKHGQVDADLNAS |
| | RTIALCKNFDINNPEIWK |
| SEQ | MSDESLVSSEDKLAIKIKIVPNAEQAKMLDEMFKKWSSICNRISRGKEDIETLRPDEGK |
| ID | ELQFNSTQLNSATMDVSDLKKAMARQGERLEAEVSKLRGRYETIDASLRDPSRRHTN |
| NO: | PQKPSSFYPSDWDISGRLTPRFHTARHYSTELRKLKAKEDKMLKTINKIKNGKIVFKPK |
| 61 | RITLWPSSVNMAFKGSRLLLKPFANGFEMELPIVISPQKTADGKSQKASAEYMRNALL |
| | GLAGYSINQLLFGMNRSQKMLANAKKPEKVEKFLEQMKNKDANFDKKIKALEGKWL |
| | LDRKLKESEKSSIAVVRTKFFKSGKVELNEDYLKLLKHMANEILERDGFVNLNKYPILS |
| | RKPMKRYKQKNIDNLKPNMWKYYIQFGYEPIFERKASGKPKNIMGIDRGLTHLLAVA |
| | VFSPDQQKFLFNHLESNPIMHWKWKLRKIRRSIQHMERRIRAEKNKHIHEAQLKKRLG |
| | SIEEKTEQHYHIVSSKIINWAIEYEAAIVLESLSHMKQRGGKKSVRTRALNYALSLFDY |
| | EKVARLITYKARIRGIPVYDVLPGMTSKTCATCLLNGSQGAYVRGLETTKAAGKATK |
| | RKNMKIGKCMVCNSSENSMIDADLNAARVIAICKYKNLNDPQPAGSRKVFKRF |
| SEQ | MLALKLKIMPTEKQAEILDAMFWKWASICSRIAKMKKKVSVKENKKELSKKIPSNSDI |
| ID | WFSKTQLCQAEVDVGDHKKALKNFEKRQESLLDELKYKVKAINEVINDESKREIDPN |
| NO: | NPSKFRIKDSTKKGNLNSPKFFTLKKWQKILQENEKRIKKKESTIEKLKRGNIFFNPTKI |
| 62 | SLHEEEYSINFGSSKLLLNCFYKYNKKSGINSDQLENKFNEFQNGLNIICSPLQPIRGSSK |
| | RSFEFIRNSIINFLMYSLYAKLFGIPRSVKALMKSNKDENKLKLEEKLKKKKSSFNKTV |
| | KEFEKMIGRKLSDNESKILNDESKKFFEIIKSNNKYIPSEEYLKLLKDISEEIYNSNIDFKP |
| | YKYSILIRKPLSKFKSKKLYNLKPTDYKYYLQLSYEPFSKQLIATKTILGIDRGLKHLLA |
| | VSVFDPSQNKFVYNKLIKNPVFKWKKRYHDLKRSIRNRERRIRALTGVHIHENQLIKK |
| | LKSMKNKINVLYHNVSKNIVDLAKKYESTIVLERLENLKQHGRSKGKRYKKLNYVLS |
| | NFDYKKIESLISYKAKKEGVPVSNINPKYTSKTCAKCLLEVNQLSELKNEYNRDSKNS |
| | KIGICNIHGQIDADLNAARVIALCYSKNLNEPHFK |
| SEQ | VINLFGYKFALYPNKTQEELLNKHLGECGWLYNKAIEQNEYYKADSNIEEAQKKFELL |
| ID | PDKNSDEAKVLRGNISKDNYVYRTLVKKKKSEINVQIRKAVVLRPAETIRNLAKVKK |
| NO: | KGLSVGRLKFIPIREWDVLPFKQSDQIRLEENYLILEPYGRLKFKMHRPLLGKPKTFCIK |
| 63 | RTATDRWTISFSTEYDDSNMRKNDGGQVGIDVGLKTHLRLSNENPDEDPRYPNPKIW |
| | KRYDRRLTILQRRISKSKKLGKNRTRLRLRLSRLWEKIRNSRADLIQNETYEILSENKLI |
| | AIEDLNVKGMQEKKDKKGRKGRTRAQEKGLHRSISDAAFSEFRRVLEYKAKRFGSEV |
| | KPVSAIDSSKECHNCGNKKGMPLESRIYECPKCGLKIDRDLNSAKVILARATGVRPGS |
| | NARADTKISATAGASVQTEGTVSEDFRQQMETSDQKPMQGEGSKEPPMNPEHKSSGR |
| | GSKHVNIGCKNKVGLYNEDENSRSTEKQIMDENRSTTEDMVEIGALHSPVLTT |
| SEQ | MIASIDYEAVSQALIVFEFKAKGKDSQYQAIDEAIRSYRFIRNSCLRYWMDNKKVGKY |
| ID | DLNKYCKVLAKQYPFANKLNSQARQSAAECSWSAISRFYDNCKRKVSGKKGFPKFK |
| NO: | KHARSVEYKTSGWKLSENRKAITFTDKNGIGKLKLKGTYDLHFSQLEDMKRVRLVRR |
| 64 | ADGYYVQFCISVDVKVETEPTGKAIGLDVGIKYFLADSSGNTIENPQFYRKAEKKLNR |
| | ANRRKSKKYIRGVKPQSKNYHKARCRYARKHLRVSRQRKEYCKRVAYCVIHSNDVV |
| | AYEDLNVKGMVKNRHLAKSISDVAWSTFRHWLEYFAIKYGKLTIPVAPHNTSQNCSN |
| | CDKKVPKSLSTRTHICHHCGYSEDRDVNAAKNILKKALSTVGQTGSLKLGEIEPLLVL |
| | EQSCTRKFDL |
| SEQ | LAEENTLHLTLAMSLPLNDLPENRTRSELWRRQWLPQKKLSLLLGVNQSVRKAAADC |
| ID | LRWFEPYQELLWWEPTDPDGKKLLDKEGRPIKRTAGHMRVLRKLEEIAPFRGYQLGS |
| NO: | AVKNGLRHKVADLLLSYAKRKLDPQFTDKTSYPSIGDQFPIVWTGAFVCYEQSITGQL |
| 65 | YLYLPLFPRGSHQEDITNNYDPDRGPALQVFGEKEIARLSRSTSGLLLPLQFDKWGEAT |
| | FIRGENNPPTWKATHRRSDKKWLSEVLLREKDFQPKRVELLVRNGRIFVNVACEIPTK |
| | PLLEVENFMGVSFGLEHLVTVVVINRDGNVVHQRQEPARRYEKTYFARLERLRRRGG |
| | PFSQELETFHYRQVAQIVEEALRFKSVPAVEQVGNIPKGRYNPRLNLRLSYWPFGKLA |
| | DLTSYKAVKEGLPKPYSVYSATAKMLCSTCGAANKEGDQPISLKGPTVYCGNCGTRH |
| | NTGFNTALNLARRAQELFVKGVVAR |
| SEQ | MSQSLLKWHDMAGRDKDASRSLQKSAVEGVLLHLTASHRVALEMLEKSVSQTVAVT |
| ID | MEAAQQRLVIVLEDDPTKATSRKRVISADLQFTREEFGSLPNWAQKLASTCPEIATKY |
| NO: | ADKHINSIRIAWGVAKESTNGDAVEQKLQWQIRLLDVTMFLQQLVLQLADKALLEQI |
| 66 | PSSIRGGIGQEVAQQVTSHIQLLDSGTVLKAELPTISDRNSELARKQWEDAIQTVCTYA |
| | LPFSRERARILDPGKYAAEDPRGDRLINIDPMWARVLKGPTVKSLPLLFVSGSSIRIVKL |
| | TLPRKHAAGHKHTFTATYLVLPVSREWINSLPGTVQEKVQWWKKPDVLATQELLVG |
| | KGALKKSANTLVIPISAGKKRFFNHILPALQRGFPLQWQRIVGRSYRRPATHRKWFAQ |
| | LTIGYTNPSSLPEMALGIHFGMKDILWWALADKQGNILKDGSIPGNSILDFSLQEKGKI |
| | ERQQKAGKNVAGKKYGKSLLNATYRVVNGVLEFSKGISAEHASQPIGLGLETIRFVDK |
| | ASGSSPVNARHSNWNYGQLSGIFANKAGPAGFSVTEITLKKAQRDLSDAEQARVLAIE |
| | ATKRFASRIKRLATKRKDDTLFV |
| SEQ | VEPVEKERFYYRTYTFRLDGQPRTQNLTTQSGWGLLTKAVLDNTKHYWEIVHHARIA |
| ID | NQPIVFENPVIDEQGNPKLNKLGQPRFWKRPISDIVNQLRALFENQNPYQLGSSLIQGT |
| NO: | YWDVAENLASWYALNKEYLAGTATWGEPSFPEPHPLTEINQWMPLTFSSGKVVRLLK |
| 67 | NASGRYFIGLPILGENNPCYRMRTIEKLIPCDGKGRVTSGSLILFPLVGIYAQQHRRMTD |
| | ICESIRTEKGKLAWAQVSIDYVREVDKRRRMRRTRKSQGWIQGPWQEVFILRLVLAH |
| | KAPKLYKPRCFAGISLGPKTLASCVILDQDERVVEKQQWSGSELLSLIHQGEERLRSLR |
| | EQSKPTWNAAYRKQLKSLINTQVFTIVTFLRERGAAVRLESIARVRKSTPAPPVNFLLS |
| | HWAYRQITERLKDLAIRNGMPLTHSNGSYGVRFTCSQCGATNQGIKDPTKYKVDIESE |
| | TFLCSICSHREIAAVNTATNLAKQLLDE |
| SEQ | MNDTETSETLTSHRTVCAHLHVVGETGSLPRLVEAALAELITLNGRATQALLSLAKNG |
| ID | LVLRRDKEENLIAAELTLPCRKNKYADVAAKAGEPILATRINNKGKLVTKKWYGEGN |
| NO: | SYHIVRFTPETGMFTVRVFDRYAFDEELLHLHSEVVFGSDLPKGIKAKTDSLPANFLQA |
| 68 | VFTSFLELPFQGFPDIVVKPAMKQAAEQLLSYVQLEAGENQQAEYPDTNERDPELRLV |
| | EWQKSLHELSVRTEPFEFVRARDIDYYAETDRRGNRFVNITPEWTKFAESPFARRLPLK |
| | IPPEFCILLRRKTEGHAKIPNRIYLGLQIFDGVTPDSTLGVLATAEDGKLFWWHDHLDE |
| | FSNLEGKPEPKLKNKPQLLMVSLEYDREQRFEESVGGDRKICLVTLKETRNFRRGWN |
| | GRILGIHFQHNPVITWALMDHDAEVLEKGFIEGNAFLGKALDKQALNEYLQKGGKWV |
| | GDRSFGNKLKGITHTLASLIVRLAREKDAWIALEEISWVQKQSADSVANHEIVEQPHH |
| | SLTR |
| SEQ | MNDTETSETLTSHRTVCAHLHVVGETGSLPRLVEAALAELITLNGRATQALLSLAKNG |
| ID | LVLRRDKEENLIAAELTLPCRKNKYADVAAKAGEPILATRINNKGKLVTKKWYGEGN |
| NO: | SYHIVRFTPETGMFTVRVFDRYAFDEELLHLHSEVVFGSDLPKGIKAKTDSLPANFLQA |
| 69 | VFTSFLELPFQGFPDIVVKPAMKQAAEQLLSYVQLEAGENQQAEYPDTNERDPELRLV |
| | EWQKSLHELSVRTEPFEFVRARDIDYYAETDRRGNRFVNITPEWTKFAESPFARRLPLK |
| | IPPEFCILLRRKTEGHAKIPNRIYLGLQIFDGVTPDSTLGVLATAEDGKLFWWHDHLDE |
| | FSNLEGKPEPKLKNKPQLLMVSLEYDREQRFEESVGGDRKICLVTLKETRNFRRGRHG |
| | HTRTDRLPAGNTLWRADFATSAEVAAPKWNGRILGIHFQHNPVITWALMDHDAEVLE |
| | KGFIEGNAFLGKALDKQALNEYLQKGGKWVGDRSFGNKLKGITHTLASLIVRLAREK |
| | DAWIALEEISWVQKQSADSVANRRFSMWNYSRLATLIEWLGTDIATRDCGTAAPLAH |
| | KVSDYLTHFTCPECGACRKAGQKKEIADTVRAGDILTCRKCGFSGPIPDNFIAEFVAKK |
| | ALERMLKKKPV |
| SEQ | MAKRNFGEKSEALYRAVRFEVRPSKEELSILLAVSEVLRMLFNSALAERQQVFTEFIAS |
| ID | LYAELKSASVPEEISEIRKKLREAYKEHSISLFDQINALTARRVEDEAFASVTRNWQEE |
| NO: | TLDALDGAYKSFLSLRRKGDYDAHSPRSRDSGFFQKIPGRSGFKIGEGRIALSCGAGRK |
| 70 | LSFPIPDYQQGRLAETTKLKKFELYRDQPNLAKSGRFWISVVYELPKPEATTCQSEQVA |
| | FVALGASSIGVVSQRGEEVIALWRSDKHWVPKIEAVEERMKRRVKGSRGWLRLLNSG |
| | KRRMHMISSRQHVQDEREIVDYLVRNHGSHFVVTELVVRSKEGKLADSSKPERGGSL |
| | GLNWAAQNTGSLSRLVRQLEEKVKEHGGSVRKHKLTLTEAPPARGAENKLWMARKL |
| | RESFLKEV |
| SEQ | LAKNDEKELLYQSVKFEIYPDESKIRVLTRVSNILVLVWNSALGERRARFELYIAPLYE |
| ID | ELKKFPRKSAESNALRQKIREGYKEHIPTFFDQLKKLLTPMRKEDPALLGSVPRAYQEE |
| NO: | TLNTLNGSFVSFMTLRRNNDMDAKPPKGRAEDRFHEISGRSGFKIDGSEFVLSTKEQK |
| 71 | LRFPIPNYQLEKLKEAKQIKKFTLYQSRDRRFWISIAYEIELPDQRPFNPEEVIYIAFGAS |
| | SIGVISPEGEKVIDFWRPDKHWKPKIKEVENRMRSCKKGSRAWKKRAAARRKMYAM |
| | TQRQQKLNHREIVASLLRLGFHFVVTEYTVRSKPGKLADGSNPKRGGAPQGFNWSAQ |
| | NTGSFGEFILWLKQKVKEQGGTVQTFRLVLGQSERPEKRGRDNKIEMVRLLREKYLES |
| | QTIVV |
| SEQ | MAKGKKKEGKPLYRAVRFEIFPTSDQITLFLRVSKNLQQVWNEAWQERQSCYEQFFG |
| ID | SIYERIGQAKKRAQEAGFSEVWENEAKKGLNKKLRQQEISMQLVSEKESLLQELSIAF |
| NO: | QEHGVTLYDQINGLTARRIIGEFALIPRNWQEETLDSLDGSFKSFLALRKNGDPDAKPP |
| 72 | RQRVSENSFYKIPGRSGFKVSNGQIYLSFGKIGQTLTSVIPEFQLKRLETAIKLKKFELCR |
| | DERDMAKPGRFWISVAYEIPKPEKVPVVSKQITYLAIGASRLGVVSPKGEFCLNLPRSD |
| | YHWKPQINALQERLEGVVKGSRKWKKRMAACTRMFAKLGHQQKQHGQYEVVKKL |
| | LRHGVHFVVTELKVRSKPGALADASKSDRKGSPTGPNWSAQNTGNIARLIQKLTDKA |
| | SEHGGTVIKRNPPLLSLEERQLPDAQRKIFIAKKLREEFLADQK |
| SEQ | MAKREKKDDVVLRGTKMRIYPTDRQVTLMDMWRRRCISLWNLLLNLETAAYGAKN |
| ID | TRSKLGWRSIWARVVEENHAKALIVYQHGKCKKDGSFVLKRDGTVKHPPRERFPGDR |
| NO: | KILLGLFDALRHTLDKGAKCKCNVNQPYALTRAWLDETGHGARTADIIAWLKDFKGE |
| 73 | CDCTAISTAAKYCPAPPTAELLTKIKRAAPADDLPVDQAILLDLFGALRGGLKQKECD |
| | HTHARTVAYFEKHELAGRAEDILAWLIAHGGTCDCKIVEEAANHCPGPRLFIWEHELA |
| | MIMARLKAEPRTEWIGDLPSHAAQTVVKDLVKALQTMLKERAKAAAGDESARKTGF |
| | PKFKKQAYAAGSVYFPNTTMFFDVAAGRVQLPNGCGSMRCEIPRQLVAELLERNLKP |
| | GLVIGAQLGLLGGRIWRQGDRWYLSCQWERPQPTLLPKTGRTAGVKIAASIVFTTYD |
| | NRGQTKEYPMPPADKKLTAVHLVAGKQNSRALEAQKEKEKKLKARKERLRLGKLEK |
| | GHDPNALKPLKRPRVRRSKLFYKSAARLAACEAIERDRRDGFLHRVTNEIVHKFDAVS |
| | VQKMSVAPMMRRQKQKEKQIESKKNEAKKEDNGAAKKPRNLKPVRKLLRHVAMAR |
| | GRQFLEYKYNDLRGPGSVLIADRLEPEVQECSRCGTKNPQMKDGRRLLRCIGVLPDGT |
| | DCDAVLPRNRNAARNAEKRLRKHREAHNA |
| SEQ | MNEVLPIPAVGEDAADTIMRGSKMRIYPSVRQAATMDLWRRRCIQLWNLLLELEQAA |
| ID | YSGENRRTQIGWRSIWATVVEDSHAEAVRVAREGKKRKDGTFRKAPSGKEIPPLDPA |
| NO: | MLAKIQRQMNGAVDVDPKTGEVTPAQPRLFMWEHELQKIMARLKQAPRTHWIDDLP |
| 74 | SHAAQSVVKDLIKALQAMLRERKKRASGIGGRDTGFPKFKKNRYAAGSVYFANTQLR |
| | FEAKRGKAGDPDAVRGEFARVKLPNGVGWMECRMPRHINAAHAYAQATLMGGRIW |
| | RQGENWYLSCQWKMPKPAPLPRAGRTAAIKIAAAIPITTVDNRGQTREYAMPPIDRER |
| | IAAHAAAGRAQSRALEARKRRAKKREAYAKKRHAKKLERGIAAKPPGRARIKLSPGF |
| | YAAAAKLAKLEAEDANAREAWLHEITTQIVRNFDVIAVPRMEVAKLMKKPEPPEEKE |
| | EQVKAPWQGKRRSLKAARVMMRRTAMALIQTTLKYKAVDLRGPQAYEEIAPLDVTA |
| | AACSGCGVLKPEWKMARAKGREIMRCQEPLPGGKTCNTVLTYTRNSARVIGRELAVR |
| | LAERQKA |
| SEQ | MTTQKTYNFCFYDQRFFELSKEAGEVYSRSLEEFWKIYDETGVWLSKFDLQKHMRNK |
| ID | LERKLLHSDSFLGAMQQVHANLASWKQAKKVVPDACPPRKPKFLQAILFKKSQIKYK |
| NO: | NGFLRLTLGTEKEFLYLKWDINIPLPIYGSVTYSKTRGWKINLCLETEVEQKNLSENKY |
| 75 | LSIDLGVKRVATIFDGENTITLSGKKFMGLMHYRNKLNGKTQSRLSHKKKGSNNYKKI |
| | QRAKRKTTDRLLNIQKEMLHKYSSFIVNYAIRNDIGNIIIGDNSSTHDSPNMRGKTNQK |
| | ISQNPEQKLKNYIKYKFESISGRVDIVPEPYTSRKCPHCKNIKKSSPKGRTYKCKKCGFI |
| | FDRDGVGAINIYNENVSFGQIISPGRIRSLTEPIGMKFHNEIYFKSYVAA |
| SEQ | MSVRSFQARVECDKQTMEHLWRTHKVFNERLPEIIKILFKMKRGECGQNDKQKSLYK |
| ID | SISQSILEANAQNADYLLNSVSIKGWKPGTAKKYRNASFTWADDAAKLSSQGIHVYD |
| NO: | KKQVLGDLPGMMSQMVCRQSVEAISGHIELTKKWEKEHNEWLKEKEKWESEDEHK |
| 76 | KYLDLREKFEQFEQSIGGKITKRRGRWHLYLKWLSDNPDFAAWRGNKAVINPLSEKA |
| | QIRINKAKPNKKNSVERDEFFKANPEMKALDNLHGYYERNFVRRRKTKKNPDGFDHK |
| | PTFTLPHPTIHPRWFVFNKPKTNPEGYRKLILPKKAGDLGSLEMRLLTGEKNKGNYPD |
| | DWISVKFKADPRLSLIRPVKGRRVVRKGKEQGQTKETDSYEFFDKHLKKWRPAKLSG |
| | VKLIFPDKTPKAAYLYFTCDIPDEPLTETAKKIQWLETGDVTKKGKKRKKKVLPHGLV |
| | SCAVDLSMRRGTTGFATLCRYENGKIHILRSRNLWVGYKEGKGCHPYRWTEGPDLGH |
| | IAKHKREIRILRSKRGKPVKGEESHIDLQKHIDYMGEDRFKKAARTIVNFALNTENAAS |
| | KNGFYPRADVLLLENLEGLIPDAEKERGINRALAGWNRRHLVERVIEMAKDAGFKRR |
| | VFEIPPYGTSQVCSKCGALGRRYSIIRENNRREIRFGYVEKLFACPNCGYCANADHNAS |
| | VNLNRRFLIEDSFKSYYDWKRLSEKKQKEEIETIESKLMDKLCAMHKISRGSISK |
| SEQ | MHLWRTHCVFNQRLPALLKRLFAMRRGEVGGNEAQRQVYQRVAQFVLARDAKDSV |
| ID | DLLNAVSLRKRSANSAFKKKATISCNGQAREVTGEEVFAEAVALASKGVFAYDKDD |
| NO: | MRAGLPDSLFQPLTRDAVACMRSHEELVATWKKEYREWRDRKSEWEAEPEHALYLN |
| 77 | LRPKFEEGEAARGGRFRKRAERDHAYLDWLEANPQLAAWRRKAPPAVVPIDEAGKR |
| | RIARAKAWKQASVRAEEFWKRNPELHALHKIHVQYLREFVRPRRTRRNKRREGFKQR |
| | PTFTMPDPVRHPRWCLFNAPQTSPQGYRLLRLPQSRRTVGSVELRLLTGPSDGAGFPD |
| | AWVNVRFKADPRLAQLRPVKVPRTVTRGKNKGAKVEADGFRYYDDQLLIERDAQVS |
| | GVKLLFRDIRMAPFADKPIEDRLLSATPYLVFAVEIKDEARTERAKAIRFDETSELTKSG |
| | KKRKTLPAGLVSVAVDLDTRGVGFLTRAVIGVPEIQQTHHGVRLLQSRYVAVGQVEA |
| | RASGEAEWSPGPDLAHIARHKREIRRLRQLRGKPVKGERSHVRLQAHIDRMGEDRFK |
| | KAARKIVNEALRGSNPAAGDPYTRADVLLYESLETLLPDAERERGINRALLRWNRAK |
| | LIEHLKRMCDDAGIRHFPVSPFGTSQVCSKCGALGRRYSLARENGRAVIRFGWVERLF |
| | ACPNPECPGRRPDRPDRPFTCNSDHNASVNLHRVFALGDQAVAAFRALAPRDSPARTL |
| | AVKRVEDTLRPQLMRVHKLADAGVDSPF |
| SEQ | MATLVYRYGVRAHGSARQQDAVVSDPAMLEQLRLGHELRNALVGVQHRYEDGKRA |
| ID | VWSGFASVAAADHRVTTGETAVAELEKQARAEHSADRTAATRQGTAESLKAARAAV |
| NO: | KQARADRKAAMAAVAEQAKPKIQALGDDRDAEIKDLYRRFCQDGVLLPRCGRCAGD |
| 78 | LRSDGDCTDCGAAHEPRKLYWATYNAIREDHQTAVKLVEAKRKAGQPARLRFRRWT |
| | GDGTLTVQLQRMHGPACRCVTCAEKLTRRARKTDPQAPAVAADPAYPPTDPPRDPAL |
| | LASGQGKWRNVLQLGTWIPPGEWSAMSRAERRRVGRSHIGWQLGGGRQLTLPVQLH |
| | RQMPADADVAMAQLTRVRVGGRHRMSVALTAKLPDPPQVQGLPPVALHLGWRQRP |
| | DGSLRVATWACPQPLDLPPAVADVVVSHGGRWGEVIMPARWLADAEVPPRLLGRRD |
| | KAMEPVLEALADWLEAHTEACTARMTPALVRRWRSQGRLAGLTNRWRGQPPTGSA |
| | EILTYLEAWRIQDKLLWERESHLRRRLAARRDDAWRRVASWLARHAGVLVVDDADI |
| | AELRRRDDPADTDPTMPASAAQAARARAALAAPGRLRHLATITATRDGLGVHTVASA |
| | GLTRLHRKCGHQAQPDPRYAASAVVTCPGCGNGYDQDYNAAMLMLDRQQQP |
| SEQ | MSRVELHRAYKFRLYPTPAQVAELAEWERQLRRLYNLAHSQRLAAMQRHVRPKSPG |
| ID | VLKSECLSCGAVAVAEIGTDGKAKKTVKHAVGCSVLECRSCGGSPDAEGRTAHTAAC |
| NO: | SFVDYYRQGREMTQLLEEDDQLARVVCSARQETLRDLEKAWQRWHKMPGFGKPHF |
| 79 | KKRIDSCRIYFSTPKSWAVDLGYLSFTGVASSVGRIKIRQDRVWPGDAKFSSCHVVRD |
| | VDEWYAVFPLTFTKEIEKPKGGAVGINRGAVHAIADSTGRVVDSPKFYARSLGVIRHR |
| | ARLLDRKVPFGRAVKPSPTKYHGLPKADIDAAAARVNASPGRLVYEARARGSIAAAE |
| | AHLAALVLPAPRQTSQLPSEGRNRERARRFLALAHQRVRRQREWFLHNESAHYAQSY |
| | TKIAIEDWSTKEMTSSEPRDAEEMKRVTRARNRSILDVGWYELGRQIAYKSEATGAEF |
| | AKVDPGLRETETHVPEAIVRERDVDVSGMLRGEAGISGTCSRCGGLLRASASGHADA |
| | ECEVCLHVEVGDVNAAVNVLKRAMFPGAAPPSKEKAKVTIGIKGRKKKRAA |
| SEQ | MSRVELHRAYKFRLYPTPVQVAELSEWERQLRRLYNLGHEQRLLTLTRHLRPKSPGV |
| ID | LKGECLSCDSTQVQEVGADGRPKTTVRHAEQCPTLACRSCGALRDAEGRTAHTVACA |
| NO: | FVDYYRQGREMTELLAADDQLARVVCSARQEVLRDLDKAWQRWRKMPGFGKPRFK |
| 80 | RRTDSCRIYFSTPKAWKLEGGHLSFTGAATTVGAIKMRQDRNWPASVQFSSCHVVRD |
| | VDEWYAVFPLTFVAEVARPKGGAVGINRGAVHAIADSTGRVVDSPRYYARALGVIRH |
| | RARLFDRKVPSGHAVKPSPTKYRGLSAIEVDRVARATGFTPGRVVTEALNRGGVAYA |
| | ECALAAIAVLGHGPERPLTSDGRNREKARKFLALAHQRVRRQREWFLHNESAHYART |
| | YSKIAIEDWSTKEMTASEPQGEETRRVTRSRNRSILDVGWYELGRQLAYKTEATGAEF |
| | AQVDPGLKETETNVPKAIADARDVDVSGMLRGEAGISGTCSKCGGLLRAPASGHADA |
| | ECEICLNVEVGDVNAAVNVLKRAMFPGDAPPASGEKPKVSIGIKGRQKKKKAA |
| SEQ | MEAIATGMSPERRVELGILPGSVELKRAYKFRLYPMKVQQAELSEWERQLRRLYNLA |
| ID | HEQRLAALLRYRDWDFQKGACPSCRVAVPGVHTAACDHVDYFRQAREMTQLLEVD |
| NO: | AQLSRVICCARQEVLRDLDKAWQRWRKKLGGRPRFKRRTDSCRIYLSTPKHWEIAGR |
| 81 | YLRLSGLASSVGEIRIEQDRAFPEGALLSSCSIVRDVDEWYACLPLTFTQPIERAPHRSV |
| | GLNRGWHALADSDGRVVDSPKFFERALATVQKRSRDLARKVSGSRNAHKARIKLA |
| | KAHQRVRRQRAAFLHQESAYYSKGFDLVALEDMSVRKMTATAGEAPEMGRGAQRD |
| | LNRGILDVGWYELARQIDYKRLAHGGELLRVDPGQTTPLACVTEEQPARGISSACAVC |
| | GIPLARPASGNARMRCTACGSSQVGDVNAAENVLTRALSSAPSGPKSPKASIKIKGRQ |
| | KRLGTPANRAGEASGGDPPVRGPVEGGTLAYVVEPVSESQSDT |
| SEQ | MTVRTYKYRAYPTPEQAEALTSWLRFASQLYNAALEHRKNAWGRHDAHGRGFRFW |
| ID | DGDAAPRKKSDPPGRWVYRGGGGAHISKNDQGKLLTEFRREHAELLPPGMPALVQH |
| NO: | EVLARLERSMAAFFQRATKGQKAGYPRWRSEHRYDSLTFGLTSPSKERFDPETGESLG |
| 82 | RGKTVGAGTYHNGDLRLTGLGELRILEHRRIPMGAIPKSVIVRRSGKRWFVSIAMEMP |
| | SVEPAASGRPAVGLDMGVVTWGTAFTADTSAAAALVADLRRMATDPSDCRRLEELE |
| | REAAQLSEVLAHCRARGLDPARPRRCPKELTKLYRRSLHRLGELDRACARIRRRLQAA |
| | HDIAEPVPDEAGSAVLIEGSNAGMRHARRVARTQRRVARRTRAGHAHSNRRKKAVQ |
| | AYARAKERERSARGDHRHKVSRALVRQFEEISVEALDIKQLTVAPEHNPDPQPDLPAH |
| | VQRRRNRGELDAAWGAFFAALDYKAADAGGRVARKPAPHTTQECARCGTLVPKPIS |
| | LRVHRCPACGYTAPRTVNSARNVLQRPLEEPGRAGPSGANGRGVPHAVA |
| SEQ | MNCRYRYRIYPTPGQRQSLARLFGCVRVVWNDALFLCRQSEKLPKNSELQKLCITQA |
| ID | KKTEARGWLGQVSAIPLQQSVADLGVAFKNFFQSRSGKRKGKKVNPPRVKRRNNRQ |
| NO: | GARFTRGGFKVKTSKVYLARIGDIKIKWSRPLPSEPSSVTVIKDCAGQYFLSFVVEVKP |
| 83 | EIKPPKNPSIGIDLGLKTFASCSNGEKIDSPDYSRLYRKLKRCQRRLAKRQRGSKRRER |
| | MRVKVAKLNAQIRDKRKDFLHKLSTKVVNENQVIALEDLNVGGMLKNRKLSRAISQ |
| | AGWYEFRSLCEGKAEKHNRDFRVISRWEPTSQVCSECGYRWGKIDLSVRSIVCINCGV |
| | EHDRDDNASVNIEQAGLKVGVGHTHDSKRTGSACKTSNGAVCVEPSTHREYVQLTLF |
| | DW |
| SEQ | MKSRWTFRCYPTPEQEQHLARTFGCVRFVWNWALRARTDAFRAGERIGYPATDKAL |
| ID | TLLKQQPETVWLNEVSSVCLQQALRDLQVAFSNFFDKRAAHPSFKRKEARQSANYTE |
| NO: | RGFSFDHERRILKLAKIGAIKVKWSRKAIPHPSSIRLIRTASGKYFVSLVVETQPAPMPE |
| 84 | TGESVGVDFGVARLATLSNGERISNPKHGAKWQRRLAFYQKRLARATKGSKRRMRIK |
| | RHVARIHEKIGNSRSDTLHKLSTDLVTRFDLICVEDLNLRGMVKNHSLARSLHDASIGS |
| | AIRMIEEKAERYGKNVVKIDRWFPSSKTCSDCGHIVEQLPLNVREWTCPECGTTHDRD |
| | ANAAANILAVGQTVSAHGGTVRRSRAKASERKSQRSANRQGVNRA |
| SEQ | KEPLNIGKTAKAVFKEIDPTSLNRAANYDASIELNCKECKFKPFKNVKRYEFNFYNNW |
| ID | YRCNPNSCLQSTYKAQVRKVEIGYEKLKNEILTQMQYYPWFGRLYQNFFHDERDKM |
| NO: | TSLDEIQVIGVQNKVFFNTVEKAWREIIKKRFKDNKETMETIPELKHAAGHGKRKLSN |
| 85 | KSLLRRRFAFVQKSFKFVDNSDVSYRSFSNNIACVLPSRIGVDLGGVISRNPKREYIPQE |
| | ISFNAFWKQHEGLKKGRNIEIQSVQYKGETVKRIEADTGEDKAWGKNRQRRFTSLILK |
| | LVPKQGGKKVWKYPEKRNEGNYEYFPIPIEFILDSGETSIRFGGDEGEAGKQKHLVIPF |
| | NDSKATPLASQQTLLENSRFNAEVKSCIGLAIYANYFYGYARNYVISSIYHKNSKNGQ |
| | AITAIYLESIAHNYVKAIERQLQNLLLNLRDFSFMESHKKELKKYFGGDLEGTGGAQK |
| | RREKEEKIEKEIEQSYLPRLIRLSLTKMVTKQVEM |
| SEQ | ELIVNENKDPLNIGKTAKAVFKEIDPTSINRAANYDASIELACKECKFKPFNNTKRHDF |
| ID | SFYSNWHRCSPNSCLQSTYRAKIRKTEIGYEKLKNEILNQMQYYPWFGRLYQNFFNDQ |
| NO: | RDKMTSLDEIQVTGVQNKIFFNTVEKAWREIIKKRFRDNKETMRTIPDLKNKSGHGSR |
| 86 | KLSNKSLLRRRFAFAQKSFKLVDNSDVSYRAFSNNVACVLPSKIGVDIGGIINKDLKRE |
| | YIPQEITFNVFWKQHDGLKKGRNIEIHSVQYKGEIVKRIEADTGEDKAWGKNRQRRFT |
| | SLILKITPKQGGKKIWKFPEKKNASDYEYFPIPIEFILDNGDASIKFGGEEGEVGKQKHL |
| | LIPFNDSKATPLSSKQMLLETSRFNAEVKSTIGLALYANYFVSYARNYVIKSTYHKNSK |
| | KGQIVTEIYLESISQNFVRAIQRQLQSLMLNLKDWGFMQTHKKELKKYFGSDLEGSKG |
| | GQKRREKEEKIEKEIEASYLPRLIRLSLTKSVTKAEEM |
| SEQ | PEEKTSKLKPNSINLAANYDANEKFNCKECKFHPFKNKKRYEFNFYNNLHGCKSCTKS |
| ID | TNNPAVKRIEIGYQKLKFEIKNQMEAYPWFGRLRINFYSDEKRKMSELNEMQVTGVK |
| NO: | NKIFFDAIECAWREILKKRFRESKETLITIPKLKNKAGHGARKHRNKKLLIRRRAFMKK |
| 87 | NFHFLDNDSISYRSFANNIACVLPSKVGVDIGGIISPDVGKDIKPVDISLNLMWASKEGI |
| | KSGRKVEIYSTQYDGNMVKKIEAETGEDKSWGKNRKRRQTSLLLSIPKPSKQVQEFDF |
| | KEWPRYKDIEKKVQWRGFPIKIIFDSNHNSIEFGTYQGGKQKVLPIPFNDSKTTPLGSK |
| | MNKLEKLRFNSKIKSRLGSAIAANKFLEAARTYCVDSLYHEVSSANAIGKGKIFIEYYL |
| | EILSQNYIEAAQKQLQRFIESIEQWFVADPFQGRLKQYFKDDLKRAKCFLCANREVQT |
| | TCYAAVKLHKSCAEKVKDKNKELAIKERNNKEDAVIKEVEASNYPRVIRLKLTKTITN |
| | KAM |
| SEQ | SESENKIIEQYYAFLYSFRDKYEKPEFKNRGDIKRKLQNKWEDFLKEQNLKNDKKLSN |
| ID | YIFSNRNFRRSYDREEENEEGIDEKKSKPKRINCFEKEKNLKDQYDKDAINASANKDG |
| NO: | AQKWGCFECIFFPMYKIESGDPNKRIIINKTRFKLFDFYLNLKGCKSCLRSTYHPYRSN |
| 88 | VYIESNYDKLKREIGNFLQQKNIFQRMRKAKVSEGKYLTNLDEYRLSCVAMHFKNRW |
| | LFFDSIQKVLRETIKQRLKQMRESYDEQAKTKRSKGHGRAKYEDQVRMIRRRAYSAQ |
| | AHKLLDNGYITLFDYDDKEINKVCLTAINQEGFDIGGYLNSDIDNVMPPIEISFHLKWK |
| | YNEPILNIESPFSKAKISDYLRKIREDLNLERGKEGKARSKKNVRRKVLASKGEDGYKK |
| | IFTDFFSKWKEELEGNAMERVLSQSSGDIQWSKKKRIHYTTLVLNINLLDKKGVGNLK |
| | YYEIAEKTKILSFDKNENKFWPITIQVLLDGYEIGTEYDEIKQLNEKTSKQFTIYDPNTKI |
| | IKIPFTDSKAVPLGMLGINIATLKTVKKTERDIKVSKIFKGGLNSKIVSKIGKGIYAGYFP |
| | TVDKEILEEVEEDTLDNEFSSKSQRNIFLKSIIKNYDKMLKEQLFDFYSFLVRNDLGVRF |
| | LTDRELQNIEDESFNLEKRFFETDRDRIARWFDNTNTDDGKEKFKKLANEIVDSYKPR |
| | LIRLPVVRVIKRIQPVKQREM |
| SEQ | KYSTRDFSELNEIQVTACKQDEFFKVIQNAWREIIKKRFLENRENFIEKKIFKNKKGRG |
| ID | KRQESDKTIQRNRASVMKNFQLIENEKIILRAPSGHVACVFPVKVGLDIGGFKTDDLEK |
| NO: | NIFPPRTITINVFWKNRDRQRKGRKLEVWGIKARTKLIEKVHKWDKLEEVKKKRLKSL |
| 89 | EQKQEKSLDNWSEVNNDSFYKVQIDELQEKIDKSLKGRTMNKILDNKAKESKEAEGL |
| | YIEWEKDFEGEMLRRIEASTGGEEKWGKRRQRRHTSLLLDIKNNSRGSKEIINFYSYAK |
| | QGKKEKKIEFFPFPLTITLDAEEESPLNIKSIPIEDKNATSKYFSIPFTETRATPLSILGDRV |
| | QKFKTKNISGAIKRNLGSSISSCKIVQNAETSAKSILSLPNVKEDNNMEIFINTMSKNYF |
| | RAMMKQMESFIFEMEPKTLIDPYKEKAIKWFEVAASSRAKRKLKKLSKADIKKSELLL |
| | SNTEEFEKEKQEKLEALEKEIEEFYLPRIVRLQLTKTILETPVM |
| SEQ | KKLQLLGHKILLKEYDPNAVNAAANFETSTAELCGQCKMKPFKNKRRFQYTFGKNY |
| ID | HGCLSCIQNVYYAKKRIVQIAKEELKHQLTDSIASIPYKYTSLFSNTNSIDELYILKQER |
| NO: | AAFFSNTNSIDELYITGIENNIAFKVISAIWDEIIKKRRQRYAESLTDTGTVKANRGHGG |
| 90 | TAYKSNTRQEKIRALQKQTLHMVTNPYISLARYKNNYIVATLPRTIGMHIGAIKDRDP |
| | QKKLSDYAINFNVFWSDDRQLIELSTVQYTGDMVRKIEAETGENNKWGENMKRTKTS |
| | LLLEILTKKTTDELTFKDWAFSTKKEIDSVTKKTYQGFPIGIIFEGNESSVKFGSQNYFPL |
| | PFDAKITPPTAEGFRLDWLRKGSFSSQMKTSYGLAIYSNKVTNAIPAYVIKNMFYKIAR |
| | AENGKQIKAKFLKKYLDIAGNNYVPFIIMQHYRVLDTFEEMPISQPKVIRLSLTKTQHII |
| | IKKDKTDSKM |
| SEQ | NTSNLINLGKKAINISANYDANLEVGCKNCKFLSSNGNFPRQTNVKEGCHSCEKSTYE |
| ID | PSIYLVKIGERKAKYDVLDSLKKFTFQSLKYQSKKSMKSRNKKPKELKEFVIFANKNK |
| NO: | AFDVIQKSYNHLILQIKKEINRMNSKKRKKNHKRRLFRDREKQLNKLRLIESSNLFLPR |
| 91 | ENKGNNHVFTYVAIHSVGRDIGVIGSYDEKLNFETELTYQLYFNDDKRLLYAYKPKQ |
| | NKIIKIKEKLWNLRKEKEPLDLEYEKPLNKSITFSIKNDNLFKVSKDLMLRRAKFNIQG |
| | KEKLSKEERKINRDLIKIKGLVNSMSYGRFDELKKEKNIWSPHIYREVRQKEIKPCLIKN |
| | GDRIEIFEQLKKKMERLRRFREKRQKKISKDLIFAERIAYNFHTKSIKNTSNKINIDQEA |
| | KRGKASYMRKRIGYETFKNKYCEQCLSKGNVYRNVQKGCSCFENPFDWIKKGDENL |
| | LPKKNEDLRVKGAFRDEALEKQIVKIAFNIAKGYEDFYDNLGESTEKDLKLKFKVGTT |
| | INEQESLKL |
| SEQ | TSNPIKLGKKAINISANYDSNLQIGCKNCKFLSYNGNFPRQTNVKEGCHSCEKSTYEPP |
| ID | VYTVRIGERRSKYDVLDSLKKFIFLSLKYRQSKKMKTRSKGIRGLEEFVISANLKKAM |
| NO: | DVIQKSYRHLILNIKNEIVRMNGKKRNKNHKRLLFRDREKQLNKLRLIEGSSFFKPPTV |
| 92 | KGDNSIFTCVAIHNIGRDIGIAGDYFDKLEPKIELTYQLYYEYNPKKESEINKRLLYAYK |
| | PKQNKIIEIKEKLWNLRKEKSPLDLEYEKPLTKSITFLVKRDGVFRISKDLMLRKAKFII |
| | QGKEKLSKEERKINRDLIKIKSNIISLTYGRFDELKKDKTIWSPHIFRDVKQGKITPCIER |
| | KGDRMDIFQQLRKKSERLRENRKKRQKKISKDLIFAERIAYNFHTKSIKNTSNLINIKHE |
| | AKRGKASYMRKRIGNETFRIKYCEQCFPKNNVYKNVQKGCSCFEDPFEYIKKGNEDLI |
| | PNKNQDLKAKGAFRDDALEKQIIKVAFNIAKGYEDFYENLKKTTEKDIRLKFKVGTIIS |
| | EEM |
| SEQ | NNSINLSKKAINISANYDANLQVRCKNCKFLSSNGNFPRQTDVKEGCHSCEKSTYEPP |
| ID | VYDVKIGEIKAKYEVLDSLKKFTFQSLKYQLSKSMKFRSKKIKELKEFVIFAKESKALN |
| NO: | VINRSYKHLILNIKNDINRMNSKKRIKNHKGRLFLDRQKQLSKLKLIEGSSFFVPAKNV |
| 93 | GNKSVFTCVAIHSIGRDIGIAGLYDSFTKPVNEITYQIFFSGERRLLYAYKPKQLKILSIK |
| | ENLWSLKNEKKPLDLLYEKPLGKNLNFNVKGGDLFRVSKDLMIRNAKFNVHGRQRLS |
| | DEERLINRNFIKIKGEVVSLSYGRFEELKKDRKLWSPHIFKDVRQNKIKPCLVMQGQRI |
| | DIFEQLKRKLELLKKIRKSRQKKLSKDLIFGERIAYNFHTKSIKNTSNKINIDSDAKRGR |
| | ASYMRKRIGNETFKLKYCDVCFPKANVYRRVQNGCSCSENPYNYIKKGDKDLLPKKD |
| | EGLAIKGAFRDEKLNKQIIKVAFNIAKGYEDFYDDLKKRTEKDVDLKFKIGTTVLDQK |
| | PMEIFDGIVITWL |
| SEQ | LLTTVVETNNLAKKAINVAANFDANIDRQYYRCTPNLCRFIAQSPRETKEKDAGCSSC |
| ID | TQSTYDPKVYVIKIGKLLAKYEILKSLKRFLFMNRYFKQKKTERAQQKQKIGTELNEM |
| NO: | SIFAKATNAMEVIKRATKHCTYDIIPETKSLQMLKRRRHRVKVRSLLKILKERRMKIKK |
| 94 | IPNTFIEIPKQAKKNKSDYYVAAALKSCGIDVGLCGAYEKNAEVEAEYTYQLYYEYKG |
| | NSSTKRILYCYNNPQKNIREFWEAFYIQGSKSHVNTPGTIRLKMEKFLSPITIESEALDF |
| | RVWNSDLKIRNGQYGFIKKRSLGKEAREIKKGMGDIKRKIGNLTYGKSPSELKSIHVY |
| | RTERENPKKPRAARKKEDNFMEIFEMQRKKDYEVNKKRRKEATDAAKIMDFAEEPIR |
| | HYHTNNLKAVRRIDMNEQVERKKTSVFLKRIMQNGYRGNYCRKCIKAPEGSNRDEN |
| | VLEKNEGCLDCIGSEFIWKKSSKEKKGLWHTNRLLRRIRLQCFTTAKAYENFYNDLFE |
| | KKESSLDIIKLKVSITTKSM |
| SEQ | ASTMNLAKQAINFAANYDSNLEIGCKGCKFMSTWSKKSNPKFYPRQNNQANKCHSCT |
| ID | YSTGEPEVPIIEIGERAAKYKIFTALKKFVFMSVAYKERRRQRFKSKKPKELKELAICSN |
| NO: | REKAMEVIQKSVVHCYGDVKQEIPRIRKIKVLKNHKGRLFYKQKRSKIKIAKLEKGSFF |
| 95 | KTFIPKVHNNGCHSCHEASLNKPILVTTALNTIGADIGLINDYSTIAPTETDISWQVYYE |
| | FIPNGDSEAVKKRLLYFYKPKGALIKSIRDKYFKKGHENAVNTGFFKYQGKIVKGPIKF |
| | VNNELDFARKPDLKSMKIKRAGFAIPSAKRLSKEDREINRESIKIKNKIYSLSYGRKKTL |
| | SDKDIIKHLYRPVRQKGVKPLEYRKAPDGFLEFFYSLKRKERRLRKQKEKRQKDMSEII |
| | DAADEFAWHRHTGSIKKTTNHINFKSEVKRGKVPIMKKRIANDSFNTRHCGKCVKQG |
| | NAINKYYIEKQKNCFDCNSIEFKWEKAALEKKGAFKLNKRLQYIVKACFNVAKAYES |
| | FYEDFRKGEEESLDLKFKIGTTTTLKQYPQNKARAM |
| SEQ | HSHNLMLTKLGKQAINFAANYDANLEIGCKNCKFLSYSPKQANPKKYPRQTDVHEDG |
| ID | NIACHSCMQSTKEPPVYIVPIGERKSKYEILTSLNKFTFLALKYKEKKRQAFRAKKPKE |
| NO: | LQELAIAFNKEKAIKVIDKSIQHLILNIKPEIARIQRQKRLKNRKGKLLYLHKRYAIKMG |
| 96 | LIKNGKYFKVGSPKKDGKKLLVLCALNTIGRDIGIIGNIEENNRSETEITYQLYFDCLDA |
| | NPNELRIKEIEYNRLKSYERKIKRLVYAYKPKQTKILEIRSKFFSKGHENKVNTGSFNFE |
| | NPLNKSISIKVKNSAFDFKIGAPFIMLRNGKFHIPTKKRLSKEEREINRTLSKIKGRVFRL |
| | TYGRNISEQGSKSLHIYRKERQHPKLSLEIRKQPDSFIDEFEKLRLKQNFISKLKKQRQK |
| | KLADLLQFADRIAYNYHTSSLEKTSNFINYKPEVKRGRTSYIKKRIGNEGFEKLYCETCI |
| | KSNDKENAYAVEKEELCFVCKAKPFTWKKTNKDKLGIFKYPSRIKDFIRAAFTVAKSY |
| | NDFYENLKKKDLKNEIFLKFKIGLILSHEKKNHISIAKSVAEDERISGKSIKNILNKSIKL |
| | EKNCYSCFFHKEDM |
| SEQ | SLERVIDKRNLAKKAINIAANFDANINKGFYRCETNQCMFIAQKPRKTNNTGCSSCLQS |
| ID | TYDPVIYVVKVGEMLAKYEILKSLKRFVFMNRSFKQKKTEKAKQKERIGGELNEMSIF |
| NO: | ANAALAMGVIKRAIRHCHVDIRPEINRLSELKKTKHRVAAKSLVKIVKQRKTKWKGIP |
| 97 | NSFIQIPQKARNKDADFYVASALKSGGIDIGLCGTYDKKPHADPRWTYQLYFDTEDES |
| | EKRLLYCYNDPQAKIRDFWKTFYERGNPSMVNSPGTIEFRMEGFFEKMTPISIESKDFD |
| | FRVWNKDLLIRRGLYEIKKRKNLNRKAREIKKAMGSVKRVLANMTYGKSPTDKKSIP |
| | VYRVEREKPKKPRAVRKEENELADKLENYRREDFLIRNRRKREATEIAKIIDAAEPPIR |
| | HYHTNHLRAVKRIDLSKPVARKNTSVFLKRIMQNGYRGNYCKKCIKGNIDPNKDECR |
| | LEDIKKCICCEGTQNIWAKKEKLYTGRINVLNKRIKQMKLECFNVAKAYENFYDNLA |
| | ALKEGDLKVLKLKVSIPALNPEASDPEEDM |
| SEQ | NASINLGKRAINLSANYDSNLVIGCKNCKFLSFNGNFPRQTNVREGCHSCDKSTYAPE |
| ID | VYIVKIGERKAKYDVLDSLKKFTFQSLKYQIKKSMRERSKKPKELLEFVIFANKDKAF |
| NO: | NVIQKSYEHLILNIKQEINRMNGKKRIKNHKKRLFKDREKQLNKLRLIGSSSLFFPREN |
| 98 | KGDKDLFTYVAIHSVGRDIGVAGSYESHIEPISDLTYQLFINNEKRLLYAYKPKQNKIIE |
| | LKENLWNLKKEKKPLDLEFTKPLEKSITFSVKNDKLFKVSKDLMLRQAKFNIQGKEKL |
| | SKEERQINRDFSKIKSNVISLSYGRFEELKKEKNIWSPHIYREVKQKEIKPCIVRKGDRIE |
| | LFEQLKRKMDKLKKFRKERQKKISKDLNFAERIAYNFHTKSIKNTSNKINIDQEAKRG |
| | KASYMRKRIGNESFRKKYCEQCFSVGNVYHNVQNGCSCFDNPIELIKKGDEGLIPKGK |
| | EDRKYKGALRDDNLQMQIIRVAFNIAKGYEDFYNNLKEKTEKDLKLKFKIGTTISTQE |
| | SNNKEM |
| SEQ | SNLIKLGKQAINFAANYDANLEVGCKNCKFLSSTNKYPRQTNVHLDNKMACRSCNQS |
| ID | TMEPAIYIVRIGEKKAKYDIYNSLTKFNFQSLKYKAKRSQRFKPKQPKELQELSIAVRK |
| NO: | EKALDIIQKSIDHLIQDIRPEIPRIKQQKRYKNHVGKLFYLQKRRKNKLNLIGKGSFFKV |
| 99 | FSPKEKKNELLVICALTNIGRDIGLIGNYNTIINPLFEVTYQLYYDYIPKKNNKNVQRRL |
| | LYAYKSKNEKILKLKEAFFKRGHENAVNLGSFSYEKPLEKSLTLKIKNDKDDFQVSPS |
| | LRIRTGRFFVPSKRNLSRQEREINRRLVKIKSKIKNMTYGKFETARDKQSVHIFRLERQ |
| | KEKLPLQFRKDEKEFMEEFQKLKRRTNSLKKLRKSRQKKLADLLQLSEKVVYNNHTG |
| | TLKKTSNFLNFSSSVKRGKTAYIKELLGQEGFETLYCSNCINKGQKTRYNIETKEKCFS |
| | CKDVPFVWKKKSTDKDRKGAFLFPAKLKDVIKATFTVAKAYEDFYDNLKSIDEKKPY |
| | IKFKIGLILAHVRHEHKARAKEEAGQKNIYNKPIKIDKNCKECFFFKEEAM |
| SEQ | NTTRKKFRKRTGFPQSDNIKLAYCSAIVRAANLDADIQKKHNQCNPNLCVGIKSNEQS |
| ID | RKYEHSDRQALLCYACNQSTGAPKVDYIQIGEIGAKYKILQMVNAYDFLSLAYNLTK |
| NO: | LRNGKSRGHQRMSQLDEVVIVADYEKATEVIKRSINHLLDDIRGQLSKLKKRTQNEHI |
| 100 | TEHKQSKIRRKLRKLSRLLKRRRWKWGTIPNPYLKNWVFTKKDPELVTVALLHKLGR |
| | DIGLVNRSKRRSKQKLLPKVGFQLYYKWESPSLNNIKKSKAKKLPKRLLIPYKNVKLF |
| | DNKQKLENAIKSLLESYQKTIKVEFDQFFQNRTEEIIAEEQQTLERGLLKQLEKKKNEF |
| | ASQKKALKEEKKKIKEPRKAKLLMEESRSLGFLMANVSYALFNTTIEDLYKKSNVVSG |
| | CIPQEPVVVFPADIQNKGSLAKILFAPKDGFRIKFSGQHLTIRTAKFKIRGKEIKILTKTK |
| | REILKNIEKLRRVWYREQHYKLKLFGKEVSAKPRFLDKRKTSIERRDPNKLADQTDDR |
| | QAELRNKEYELRHKQHKMAERLDNIDTNAQNLQTLSFWVGEADKPPKLDEKDARGF |
| | GVRTCISAWKWFMEDLLKKQEEDPLLKLKLSIM |
| SEQ | PKKPKFQKRTGFPQPDNLRKEYCLAIVRAANLDADFEKKCTKCEGIKTNKKGNIVKGR |
| ID | TYNSADKDNLLCYACNISTGAPAVDYVFVGALEAKYKILQMVKAYDFHSLAYNLAK |
| NO: | LWKGRGRGHQRMGGLNEVVIVSNNEKALDVIEKSLNHFHDEIRGELSRLKAKFQNEH |
| 101 | LHVHKESKLRRKLRKISRLLKRRRWKWDVIPNSYLRNFTFTKTRPDFISVALLHRVGR |
| | DIGLVTKTKIPKPTDLLPQFGFQIYYTWDEPKLNKLKKSRLRSEPKRLLVPYKKIELYK |
| | NKSVLEEAIRHLAEVYTEDLTICFKDFFETQKRKFVSKEKESLKRELLKELTKLKKDFS |
| | ERKTALKRDRKEIKEPKKAKLLMEESRSLGFLAANTSYALFNLIAADLYTKSKKACST |
| | KLPRQLSTILPLEIKEHKSTTSLAIKPEEGFKIRFSNTHLSIRTPKFKMKGADIKALTKRK |
| | REILKNATKLEKSWYGLKHYKLKLYGKEVAAKPRFLDKRNPSIDRRDPKELMEQIEN |
| | RRNEVKDLEYEIRKGQHQMAKRLDNVDTNAQNLQTKSFWVGEADKPPELDSMEAK |
| | KLGLRTCISAWKWFMKDLVLLQEKSPNLKLKLSLTEM |
| SEQ | KFSKRQEGFLIPDNIDLYKCLAIVRSANLDADVQGHKSCYGVKKNGTYRVKQNGKKG |
| ID | VKEKGRKYVFDLIAFKGNIEKIPHEAIEEKDQGRVIVLGKFNYKLILNIEKNHNDRASL |
| NO: | EIKNKIKKLVQISSLETGEFLSDLLSGKIGIDEVYGIIEPDVFSGKELVCKACQQSTYAPL |
| 102 | VEYMPVGELDAKYKILSAIKGYDFLSLAYNLSRNRANKKRGHQKLGGGELSEVVISA |
| | NYDKALNVIKRSINHYHVEIKPEISKLKKKMQNEPLKVMKQARIRRELHQLSRKVKRL |
| | KWKWGMIPNPELQNIIFEKKEKDFVSYALLHTLGRDIGLFKDTSMLQVPNISDYGFQIY |
| | YSWEDPKLNSIKKIKDLPKRLLIPYKRLDFYIDTILVAKVIKNLIELYRKSYVYETFGEE |
| | YGYAKKAEDILFDWDSINLSEGIEQKIQKIKDEFSDLLYEARESKRQNFVESFENILGLY |
| | DKNFASDRNSYQEKIQSMIIKKQQENIEQKLKREFKEVIERGFEGMDQNKKYYKVLSP |
| | NIKGGLLYTDTNNLGFFRSHLAFMLLSKISDDLYRKNNLVSKGGNKGILDQTPETMLT |
| | LEFGKSNLPNISIKRKFFNIKYNSSWIGIRKPKFSIKGAVIREITKKVRDEQRLIKSLEGV |
| | WHKSTHFKRWGKPRFNLPRHPDREKNNDDNLMESITSRREQIQLLLREKQKQQEKMA |
| | GRLDKIDKEIQNLQTANFQIKQIDKKPALTEKSEGKQSVRNALSAWKWFMEDLIKYQ |
| | KRTPILQLKLAKM |
| SEQ | KFSKRQEGFVIPENIGLYKCLAIVRSANLDADVQGHVSCYGVKKNGTYVLKQNGKKSI |
| ID | REKGRKYASDLVAFKGDIEKIPFEVIEEKKKEQSIVLGKFNYKLVLDVMKGEKDRASL |
| NO: | TMKNKSKKLVQVSSLGTDEFLLTLLNEKFGIEEIYGIIEPEVFSGKKLVCKACQQSTYA |
| 103 | PLVEYMPVGELDSKYKILSAIKGYDFLSLAYNLARHRSNKKRGHQKLGGGELSEVVIS |
| | ANNAKALNVIKRSLNHYYSEIKPEISKLRKKMQNEPLKVGKQARMRRELHQLSRKVK |
| | RLKWKWGKIPNLELQNITFKESDRDFISYALLHTLGRDIGMFNKTEIKMPSNILGYGFQ |
| | IYYDWEEPKLNTIKKSKNTPKRILIPYKKLDFYNDSILVARAIKELVGLFQESYEWEIFG |
| | NEYNYAKEAEVELIKLDEESINGNVEKKLQRIKENFSNLLEKAREKKRQNFIESFESIAR |
| | LYDESFTADRNEYQREIQSFIIEKQKQSIEKKLKNEFKKIVEKKFNEQEQGKKHYRVLN |
| | PTIINEFLPKDKNNLGFLRSKIAFILLSKISDDLYKKSNAVSKGGEKGIIKQQPETILDLEF |
| | SKSKLPSINIKKKLFNIKYTSSWLGIRKPKFNIKGAKIREITRRVRDVQRTLKSAESSWY |
| | ASTHFRRWGFPRFNQPRHPDKEKKSDDRLIESITLLREQIQILLREKQKGQKEMAGRLD |
| | DVDKKIQNLQTANFQIKQTGDKPALTEKSAGKQSFRNALSAWKWFMENLLKYQNKT |
| | PDLKLKIARTVM |
| SEQ | KWIEPNNIDFNKCLAITRSANLDADVQGHKMCYGIKTNGTYKAIGKINKKHNTGIIEK |
| ID | RRTYVYDLIVTKEKNEKIVKKTDFMAIDEEIEFDEKKEKLLKKYIKAEVLGTGELIRKD |
| NO: | LNDGEKFDDLCSIEEPQAFRRSELVCKACNQSTYASDIRYIPIGEIEAKYKILKAIKGYD |
| 104 | FLSLKYNLGRLRDSKKRGHQKMGQGELKEFVICANKEKALDVIKRSLNHYLNEVKDE |
| | ISRLNKKMQNEPLKVNDQARWRRELNQISRRLKRLKWKWGEIPNPELKNLIFKSSRPE |
| | FVSYALIHTLGRDIGLINETELKPNNIQEYGFQIYYKWEDPELNHIKKVKNIPKRFIIPYK |
| | NLDLFGKYTILSRAIEGILKLYSSSFQYKSFKDPNLFAKEGEKKITNEDFELGYDEKIKKI |
| | KDDFKSYKKALLEKKKNTLEDSLNSILSVYEQSLLTEQINNVKKWKEGLLKSKESIHK |
| | QKKIENIEDIISRIEELKNVEGWIRTKERDIVNKEETNLKREIKKELKDSYYEEVRKDFS |
| | DLKKGEESEKKPFREEPKPIVIKDYIKFDVLPGENSALGFFLSHLSFNLFDSIQYELFEKS |
| | RLSSSKHPQIPETILDL |
| SEQ | FRKFVKRSGAPQPDNLNKYKCIAIVRAANLDADIMSNESSNCVMCKGIKMNKRKTAK |
| ID | GAAKTTELGRVYAGQSGNLLCTACTKSTMGPLVDYVPIGRIRAKYTILRAVKEYDFLS |
| NO: | LAYNLARTRVSKKGGRQKMHSLSELVIAAEYEIAWNIIKSSVIHYHQETKEEISGLRKK |
| 105 | LQAEHIHKNKEARIRREMHQISRRIKRLKWKWHMIPNSELHNFLFKQQDPSFVAVALL |
| | HTLGRDIGMINKPKGSAKREFIPEYGFQIYYKWMNPKLNDINKQKYRKMPKRSLIPYK |
| | NLNVFGDRELIENAMHKLLKLYDENLEVKGSKFFKTRVVAISSKESEKLKRDLLWKG |
| | ELAKIKKDFNADKNKMQELFKEVKEPKKANALMKQSRNMGFLLQNISYGALGLLAN |
| | RMYEASAKQSKGDATKQPSIVIPLEMEFGNAFPKLLLRSGKFAMNVSSPWLTIRKPKF |
| | VIKGNKIKNITKLMKDEKAKLKRLETSYHRATHFRPTLRGSIDWDSPYFSSPKQPNTHR |
| | RSPDRLSADITEYRGRLKSVEAELREGQRAMAKKLDSVDMTASNLQTSNFQLEKGED |
| | PRLTEIDEKGRSIRNCISSWKKFMEDLMKAQEANPVIKIKIALKDESSVLSEDSM |
| SEQ | KFHPENLNKSYCLAIVRAANLDADIQGHINCIGIKSNKSDRNYENKLESLQNVELLCKA |
| ID | CTKSTYKPNINSVPVGEKKAKYSILSEIKKYDFNSLVYNLKKYRKGKSRGHQKLNELR |
| NO: | ELVITSEYKKALDVINKSVNHYLVNIKNKMSKLKKILQNEHIHVGTLARIRRERNRISR |
| 106 | KLDHYRKKWKFVPNKILKNYVFKNQSPDFVSVALLHKLGRDIGLITKTAILQKSFPEY |
| | SLQLYYKYDTPKLNYLKKSKFKSLPKRILISYKYPKFDINSNYIEESIDKLLKLYEESPIY |
| | KNNSKIIEFFKKSEDNLIKSENDSLKRGIMKEFEKVTKNFSSKKKKLKEELKLKNEDKN |
| | SKMLAKVSRPIGFLKAYLSYMLFNIISNRIFEFSRKSSGRIPQLPSCIINLGNQFENFKNEL |
| | QDSNIGSKKNYKYFCNLLLKSSGFNISYEEEHLSIKTPNFFINGRKLKEITSEKKKIRKEN |
| | EQLIKQWKKLTFFKPSNLNGKKTSDKIRFKSPNNPDIERKSEDNIVENIAKVKYKLEDL |
| | LSEQRKEFNKLAKKHDGVDVEAQCLQTKSFWIDSNSPIKKSLEKKNEKVSVKKKMKA |
| | IRSCISAWKWFMADLIEAQKETPMIKLKLALM |
| SEQ | TTLVPSHLAGIEVMDETTSRNEDMIQKETSRSNEDENYLGVKNKCGINVHKSGRGSSK |
| ID | HEPNMPPEKSGEGQMPKQDSTEMQQRFDESVTGETQVSAGATASIKTDARANSGPRV |
| NO: | GTARALIVKASNLDRDIKLGCKPCEYIRSELPMGKKNGCNHCEKSSDIASVPKVESGFR |
| 107 | KAKYELVRRFESFAADSISRHLGKEQARTRGKRGKKDKKEQMGKVNLDEIAILKNES |
| | LIEYTENQILDARSNRIKEWLRSLRLRLRTRNKGLKKSKSIRRQLITLRRDYRKWIKPNP |
| | YRPDEDPNENSLRLHTKLGVDIGVQGGDNKRMNSDDYETSFSITWRDTATRKICFTKP |
| | KGLLPRHMKFKLRGYPELILYNEELRIQDSQKFPLVDWERIPIFKLRGVSLGKKKVKAL |
| | NRITEAPRLVVAKRIQVNIESKKKKVLTRYVYNDKSINGRLVKAEDSNKDPLLEFKKQ |
| | AEEINSDAKYYENQEIAKNYLWGCEGLHKNLLEEQTKNPYLAFKYGFLNIV |
| SEQ | LDFKRTCSQELVLLPEIEGLKLSGTQGVTSLAKKLINKAANVDRDESYGCHHCIHTRTS |
| ID | LSKPVKKDCNSCNQSTNHPAVPITLKGYKIAFYELWHRFTSWAVDSISKALHRNKVM |
| NO: | GKVNLDEYAVVDNSHIVCYAVRKCYEKRQRSVRLHKRAYRCRAKHYNKSQPKVGRI |
| 108 | YKKSKRRNARNLKKEAKRYFQPNEITNGSSDALFYKIGVDLGIAKGTPETEVKVDVSI |
| | CFQVYYGDARRVLRVRKMDELQSFHLDYTGKLKLKGIGNKDTFTIAKRNESLKWGST |
| | KYEVSRAHKKFKPFGKKGSVKRKCNDYFRSIASWSCEAASQRAQSNLKNAFPYQKAL |
| | VKCYKNLDYKGVKKNDMWYRLCSNRIFRYSRIAEDIAQYQSDKGKAKFEFVILAQSV |
| | AEYDISAIM |
| SEQ | VFLTDDKRKTALRKIRSAFRKTAEIALVRAQEADSLDRQAKKLTIETVSFGAPGAKNA |
| ID | FIGSLQGYNWNSHRANVPSSGSAKDVFRITELGLGIPQSAHEASIGKSFELVGNVVRYT |
| NO: | ANLLSKGYKKGAVNKGAKQQREIKGKEQLSFDLISNGPISGDKLINGQKDALAWWLI |
| 109 | DKMGFHIGLAMEPLSSPNTYGITLQAFWKRHTAPRRYSRGVIRQWQLPFGRQLAPLIH |
| | NFFRKKGASIPIVLTNASKKLAGKGVLLEQTALVDPKKWWQVKEQVTGPLSNIWERS |
| | VPLVLYTATFTHKHGAAHKRPLTLKVIRISSGSVFLLPLSKVTPGKLVRAWMPDINILR |
| | DGRPDEAAYKGPDLIRARERSFPLAYTCVTQIADEWQKRALESNRDSITPLEAKLVTG |
| | SDLLQIHSTVQQAVEQGIGGRISSPIQELLAKDALQLVLQQLFMTVDLLRIQWQLKQEV |
| | ADGNTSEKAVGWAIRISNIHKDAYKTAIEPCTSALKQAWNPLSGFEERTFQLDASIVRK |
| | RSTAKTPDDELVIVLRQQAAEMTVAVTQSVSKELMELAVRHSATLHLLVGEVASKQL |
| | SRSADKDRGAMDHWKLLSQSM |
| SEQ | EDLLQKALNTATNVAAIERHSCISCLFTESEIDVKYKTPDKIGQNTAGCQSCTFRVGYS |
| ID | GNSHTLPMGNRIALDKLRETIQRYAWHSLLFNVPPAPTSKRVRAISELRVAAGRERLFT |
| NO: | VITFVQTNILSKLQKRYAANWTPKSQERLSRLREEGQHILSLLESGSWQQKEVVREDQ |
| 110 | DLIVCSALTKPGLSIGAFCRPKYLKPAKHALVLRLIFVEQWPGQIWGQSKRTRRMRRR |
| | KDVERVYDISVQAWALKGKETRISECIDTMRRHQQAYIGVLPFLILSGSTVRGKGDCPI |
| | LKEITRMRYCPNNEGLIPLGIFYRGSANKLLRVVKGSSFTLPMWQNIETLPHPEPFSPEG |
| | WTATGALYEKNLAYWSALNEAVDWYTGQILSSGLQYPNQNEFLARLQNVIDSIPRKW |
| | FRPQGLKNLKPNGQEDIVPNEFVIPQNAIRAHHVIEWYHKTNDLVAKTLLGWGSQTTL |
| | NQTRPQGDLRFTYTRYYFREKEVPEV |
| SEQ | VPKKKLMRELAKKAVFEAIFNDPIPGSFGCKRCTLIDGARVTDAIEKKQGAKRCAGCE |
| ID | PCTFHTLYDSVKHALPAATGCDRTAIDTGLWEILTALRSYNWMSFRRNAVSDASQKQ |
| NO: | VWSIEELAIWADKERALRVILSALTHTIGKLKNGFSRDGVWKGGKQLYENLAQKDLA |
| 111 | KGLFANGEIFGKELVEADHDMLAWTIVPNHQFHIGLIRGNWKPAAVEASTAFDARWL |
| | TNGAPLRDTRTHGHRGRRFNRTEKLTVLCIKRDGGVSEEFRQERDYELSVMLLQPKN |
| | KLKPEPKGELNSFEDLHDHWWFLKGDEATALVGLTSDPTVGDFIQLGLYIRNPIKAHG |
| | ETKRRLLICFEPPIKLPLRRAFPSEAFKTWEPTINVFRNGRRDTEAYYDIDRARVFEFPE |
| | TRVSLEHLSKQWEVLRLEPDRENTDPYEAQQNEGAELQVYSLLQEAAQKMAPKVVID |
| | PFGQFPLELFSTFVAQLFNAPLSDTKAKIGKPLDSGFVVESHLHLLEEDFAYRDFVRVT |
| | FMGTEPTFRVIHYSNGEGYWKKTVLKGKNNIRTALIPEGAKAAVDAYKNKRCPLTLE |
| | AAILNEEKDRRLVLGNKALSLLAQTARGNLTILEALAAEVLRPLSGTEGVVHLHACVT |
| | RHSTLTESTETDNM |
| SEQ | VEKLFSERLKRAMWLKNEAGRAPPAETLTLKHKRVSGGHEKVKEELQRVLRSLSGTN |
| ID | QAAWNLGLSGGREPKSSDALKGEKSRVVLETVVFHSGHNRVLYDVIEREDQVHQRSS |
| NO: | IMHMRRKGSNLLRLWGRSGKVRRKMREEVAEIKPVWHKDSRWLAIVEEGRQSVVGI |
| 112 | SSAGLAVFAVQESQCTTAEPKPLEYVVSIWFRGSKALNPQDRYLEFKKLKTTEALRGQ |
| | QYDPIPFSLKRGAGCSLAIRGEGIKFGSRGPIKQFFGSDRSRPSHADYDGKRRLSLFSKY |
| | AGDLADLTEEQWNRTVSAFAEDEVRRATLANIQDFLSISHEKYAERLKKRIESIEEPVS |
| | ASKLEAYLSAIFETFVQQREALASNFLMRLVESVALLISLEEKSPRVEFRVARYLAESK |
| | EGFNRKAM |
| SEQ | VVITQSELYKERLLRVMEIKNDRGRKEPRESQGLVLRFTQVTGGQEKVKQKLWLIFEG |
| ID | FSGTNQASWNFGQPAGGRKPNSGDALKGPKSRVTYETVVFHFGLRLLSAVIERHNLK |
| NO: | QQRQTMAYMKRRAAARKKWARSGKKCSRMRNEVEKIKPKWHKDPRWFDIVKEGEP |
| 113 | SIVGISSAGFAIYIVEEPNFPRQDPLEIEYAISIWFRRDRSQYLTFKKIQKAEKLKELQYN |
| | PIPFRLKQEKTSLVFESGDIKFGSRGSIEHFRDEARGKPPKADMDNNRRLTMFSVFSGN |
| | LTNLTEEQYARPVSGLLAPDEKRMPTLLKKLQDFFTPIHEKYGERIKQRLANSEASKRP |
| | FKKLEEYLPAIYLEFRARREGLASNWVLVLINSVRTLVRIKSEDPYIEFKVSQYLLEKE |
| | DNKAL |
| SEQ | KQDALFEERLKKAIFIKRQADPLQREELSLLPPNRKIVTGGHESAKDTLKQILRAINGTN |
| ID | QASWNPGTPSGKRDSKSADALAGPKSRVKLETVVFHVGHRLLKKVVEYQGHQKQQH |
| NO: | GLKAFMRTCAAMRKKWKRSGKVVGELREQLANIQPKWHYDSRPLNLCFEGKPSVVG |
| 114 | LRSAGIALYTIQKSVVPVKEPKPIEYAVSIWFRGPKAMDREDRCLEFKKLKIATELRKL |
| | QFEPIVSTLTQGIKGFSLYIQGNSVKFGSRGPIKYFSNESVRQRPPKADPDGNKRLALFS |
| | KFSGDLSDLTEEQWNRPILAFEGIIRRATLGNIQDYLTVGHEQFAISLEQLLSEKESVLQ |
| | MSIEQQRLKKNLGKKAENEWVESFGAEQARKKAQGIREYISGFFQEYCSQREQWAEN |
| | WVQQLNKSVRLFLTIQDSTPFIEFRVARYLPKGEKKKGKAM |
| SEQ | ANHAERHKRLRKEANRAANRNRPLVADCDTGDPLVGICRLLRRGDKMQPNKTGCRS |
| ID | CEQVEPELRDAILVSGPGRLDNYKYELFQRGRAMAVHRLLKRVPKLNRPKKAAGNDE |
| NO: | KKAENKKSEIQKEKQKQRRMMPAVSMKQVSVADFKHVIENTVRHLFGDRRDREIAE |
| 115 | CAALRAASKYFLKSRRVRPRKLPKLANPDHGKELKGLRLREKRAKLKKEKEKQAELA |
| | RSNQKGAVLHVATLKKDAPPMPYEKTQGRNDYTTFVISAAIKVGATRGTKPLLTPQP |
| | REWQCSLYWRDGQRWIRGGLLGLQAGIVLGPKLNRELLEAVLQRPIECRMSGCGNPL |
| | QVRGAAVDFFMTTNPFYVSGAAYAQKKFKPFGTKRASEDGAAAKAREKLMTQLAK |
| | VLDKVVTQAAHSPLDGIWETRPEAKLRAMIMALEHEWIFLRPGPCHNAAEEVIKCDC |
| | TGGHAILWALIDEARGALEHKEFYAVTRAHTHDCEKQKLGGRLAGFLDLLIAQDVPL |
| | DDAPAARKIKTLLEATPPAPCYKAATSIATCDCEGKFDKLWAIIDATRAGHGTEDLWA |
| | RTLAYPQNVNCKCKAGKDLTHRLADFLGLLIKRDGPFRERPPHKVTGDRKLVFSGDK |
| | KCKGHQYVILAKAHNEEVVRAWISRWGLKSRTNKAGYAATELNLLLNWLSICRRRW |
| | MDMLTVQRDTPYIRMKTGRLVVDDKKERKAM |
| SEQ | AKQREALRVALERGIVRASNRTYTLVTNCTKGGPLPEQCRMIERGKARAMKWEPKLV |
| ID | GCGSCAAATVDLPAIEEYAQPGRLDVAKYKLTTQILAMATRRMMVRAAKLSRRKGQ |
| NO: | WPAKVQEEKEEPPEPKKMLKAVEMRPVAIVDFNRVIQTTIEHLWAERANADEAELKA |
| 116 | LKAAAAYFGPSLKIRARGPPKAAIGRELKKAHRKKAYAERKKARRKRAELARSQARG |
| | AAAHAAIRERDIPPMAYERTQGRNDVTTIPIAAAIKIAATRGARPLPAPKPMKWQCSL |
| | YWNEGQRWIRGGMLTAQAYAHAANIHRPMRCEMWGVGNPLKVRAFEGRVADPDG |
| | AKGRKAEFRLQTNAFYVSGAAYRNKKFKPFGTDRGGIGSARKKRERLMAQLAKILDK |
| | VVSQAAHSPLDDIWHTRPAQKLRAMIKQLEHEWMFLRPQAPTVEGTKPDVDVAGNM |
| | QRQIKALMAPDLPPIEKGSPAKRFTGDKRKKGERAVRVAEAHSDEVVTAWISRWGIQ |
| | TRRNEGSYAAQELELLLNWLQICRRRWLDMTAAQRVSPYIRMKSGRMITDAADEGV |
| | APIPLVENM |
| SEQ | KSISGRSIKHMACLKDMLKSEITEIEEKQKKESLRKWDYYSKFSDEILFRRNLNVSANH |
| ID | DANACYGCNPCAFLKEVYGFRIERRNNERIISYRRGLAGCKSCVQSTGYPPIEFVRRKF |
| NO: | GADKAMEIVREVLHRRNWGALARNIGREKEADPILGELNELLLVDARPYFGNKSAAN |
| 117 | ETNLAFNVITRAAKKFRDEGMYDIHKQLDIHSEEGKVPKGRKSRLIRIERKHKAIHGLD |
| | PGETWRYPHCGKGEKYGVWLNRSRLIHIKGNEYRCLTAFGTTGRRMSLDVACSVLGH |
| | PLVKKKRKKGKKTVDGTELWQIKKATETLPEDPIDCTFYLYAAKPTKDPFILKVGSLK |
| | APRWKKLHKDFFEYSDTEKTQGQEKGKRVVRRGKVPRILSLRPDAKFKVSIWDDPYN |
| | GKNKEGTLLRMELSGLDGAKKPLILKRYGEPNTKPKNFVFWRPHITPHPLTFTPKHDF |
| | GDPNKKTKRRRVFNREYYGHLNDLAKMEPNAKFFEDREVSNKKNPKAKNIRIQAKES |
| | LPNIVAKNGRWAAFDPNDSLWKLYLHWRGRRKTIKGGISQEFQEFKERLDLYKKHED |
| | ESEWKEKEKLWENHEKEWKKTLEIHGSIAEVSQRCVMQSMMGPLDGLVQKKDYVHI |
| | GQSSLKAADDAWTFSANRYKKATGPKWGKISVSNLLYDANQANAELISQSISKYLSK |
| | QKDNQGCEGRKMKFLIKIIEPLRENFVKHTRWLHEMTQKDCEVRAQFSRVSM |
| SEQ | FPSDVGADALKHVRMLQPRLTDEVRKVALTRAPSDRPALARFAAVAQDGLAFVRHL |
| ID | NVSANHDSNCTFPRDPRDPRRGPCEPNPCAFLREVWGFRIVARGNERALSYRRGLAGC |
| NO: | KSCVQSTGFPSVPFHRIGADDCMRKLHEILKARNWRLLARNIGREREADPLLTELSEYL |
| 118 | LVDARTYPDGAAPNSGRLAENVIKRAAKKFRDEGMRDIHAQLRVHSREGKVPKGRL |
| | QRLRRIERKHRAIHALDPGPSWEAEGSARAEVQGVAVYRSQLLRVGHHTQQIEPVGIV |
| | ARTLFGVGRTDLDVAVSVLGAPLTKRKKGSKTLESTEDFRIAKARETRAEDKIEVAFV |
| | LYPTASLLRDEIPKDAFPAMRIDRFLLKVGSVQADREILLQDDYYRFGDAEVKAGKNK |
| | GRTVTRPVKVPRLQALRPDAKFRVNVWADPFGAGDSPGTLLRLEVSGVTRRSQPLRL |
| | LRYGQPSTQPANFLCWRPHRVPDPMTFTPRQKFGERRKNRRTRRPRVFERLYQVHIKH |
| | LAHLEPNRKWFEEARVSAQKWAKARAIRRKGAEDIPVVAPPAKRRWAALQPNAELW |
| | DLYAHDREARKRFRGGRAAEGEEFKPRLNLYLAHEPEAEWESKRDRWERYEKKWTA |
| | VLEEHSRMCAVADRTLPQFLSDPLGARMDDKDYAFVGKSALAVAEAFVEEGTVERA |
| | QGNCSITAKKKFASNASRKRLSVANLLDVSDKADRALVFQAVRQYVQRQAENGGVE |
| | GRRMAFLRKLLAPLRQNFVCHTRWLHM |
| SEQ | AARKKKRGKIGITVKAKEKSPPAAGPFMARKLVNVAANVDGVEVHLCVECEADAHG |
| ID | SASARLLGGCRSCTGSIGAEGRLMGSVDVDRERVIAEPVHTETERLGPDVKAFEAGTA |
| NO: | ESKYAIQRGLEYWGVDLISRNRARTVRKMEEADRPESSTMEKTSWDEIAIKTYSQAYH |
| 119 | ASENHLFWERQRRVRQHALALFRRARERNRGESPLQSTQRPAPLVLAALHAEAAAIS |
| | GRARAEYVLRGPSANVRAAAADIDAKPLGHYKTPSPKVARGFPVKRDLLRARHRIVG |
| | LSRAYFKPSDVVRGTSDAIAHVAGRNIGVAGGKPKEIEKTFTLPFVAYWEDVDRVVH |
| | CSSFKADGPWVRDQRIKIRGVSSAVGTFSLYGLDVAWSKPTSFYIRCSDIRKKFHPKGF |
| | GPMKHWRQWAKELDRLTEQRASCVVRALQDDEELLQTMERGQRYYDVFSCAATHA |
| | TRGEADPSGGCSRCELVSCGVAHKVTKKAKGDTGIEAVAVAGCSLCESKLVGPSKPR |
| | VHRQMAALRQSHALNYLRRLQREWEALEAVQAPTPYLRFKYARHLEVRSM |
| SEQ | AAKKKKQRGKIGISVKPKEGSAPPADGPFMARKLVNVAANVDGVEVNLCIECEADAH |
| ID | GSAPARLLGGCKSCTGSIGAEGRLMGSVDVDRADAIAKPVNTETEKLGPDVQAFEAG |
| NO: | TAETKYALQRGLEYWGVDLISRNRSRTVRRTEEGQPESATMEKTSWDEIAIKSYTRAY |
| 120 | HASENHLFWERQRRVRQHALALFKRAKERNRGDSTLPREPGHGLVAIAALACEAYAV |
| | GGRNLAETVVRGPTFGTARAVRDVEIASLGRYKTPSPKVAHGSPVKRDFLRARHRIVG |
| | LARAYYRPSDVVRGTSDAIAHVAGRNIGVAGGKPRAVEAVFTLPFVAYWEDVDRVV |
| | HCSSFQVSAPWNRDQRMKIAGVTTAAGTFSLHGGELKWAKPTSFYIRCSDTRRKFRP |
| | KGFGPMKRWRQWAKDLDRLVEQRASCVVRALQDDAALLETMERGQRYYDVFACA |
| | VTHATRGEADRLAGCSRCALTPCQEAHRVTTKPRGDAGVEQVQTSDCSLCEGKLVGP |
| | SKPRLHRTLTLLRQEHGLNYLRRLQREWESLEAVQVPTPYLRFKYARHLEVRSM |
| SEQ | TDSQSESVPEVVYALTGGEVPGRVPPDGGSAEGARNAPTGLRKQRGKIKISAKPSKPG |
| ID | SPASSLARTLVNEAANVDGVQSSGCATCRMRANGSAPRALPIGCVACASSIGRAPQEE |
| NO: | TVCALPTTQGPDVRLLEGGHALRKYDIQRALEYWGVDLIGRNLDRQAGRGMEPAEG |
| 121 | ATATMKRVSMDELAVLDFGKSYYASEQHLFAARQRRVRQHAKALKIRAKHANRSGS |
| | VKRALDRSRKQVTALAREFFKPSDVVRGDSDALAHVVGRNLGVSRHPAREIPQTFTLP |
| | LCAYWEDVDRVISCSSLLAGEPFARDQEIRIEGVSSALGSLRLYRGAIEWHKPTSLYIR |
| | CSDTRRKFRPRGGLKKRWRQWAKDLDRLVEQRACCIVRSLQADVELLQTMERAQRF |
| | YDVHDCAATHVGPVAVRCSPCAGKQFDWDRYRLLAALRQEHALNYLRRLQREWES |
| | LEAQQVKMPYLRFKYARKLEVSGPLIGLEVRREPSMGTAIAEM |
| SEQ | AGTAGRRHGSLGARRSINIAGVTDRHGRWGCESCVYTRDQAGNRARCAPCDQSTYA |
| ID | PDVQEVTIGQRQAKYTIFLTLQSFSWTNTMRNNKRAAAGRSKRTTGKRIGQLAEIKIT |
| NO: | GVGLAHAHNVIQRSLQHNITKMWRAEKGKSKRVARLKKAKQLTKRRAYFRRRMSR |
| 122 | QSRGNGFFRTGKGGIHAVAPVKIGLDVGMIASGSSEPADEQTVTLDAIWKGRKKKIRL |
| | IGAKGELAVAACRFREQQTKGDKCIPLILQDGEVRWNQNNWQCHPKKLVPLCGLEVS |
| | RKFVSQADRLAQNKVASPLAARFDKTSVKGTLVESDFAAVLVNVTSIYQQCHAMLLR |
| | SQEPTPSLRVQRTITSM |
| SEQ | GVRFSPAQSQVFFRTVIPQSVEARFAINMAAIHDAAGAFGCSVCRFEDRTPRNAKAVH |
| ID | GCSPCTRSTNRPDVFVLPVGAIKAKYDVFMRLLGFNWTHLNRRQAKRVTVRDRIGQL |
| NO: | DELAISMLTGKAKAVLKKSICHNVDKSFKAMRGSLKKLHRKASKTGKSQLRAKLSDL |
| 123 | RERTNTTQEGSHVEGDSDVALNKIGLDVGLVGKPDYPSEESVEVVVCLYFVGKVLILD |
| | AQGRIRDMRAKQYDGFKIPIIQRGQLTVLSVKDLGKWSLVRQDYVLAGDLRFEPKISK |
| | DRKYAECVKRIALITLQASLGFKERIPYYVTKQVEIKNASHIAFVTEAIQNCAENFREM |
| | TEYLMKYQEKSPDLKVLLTQLM |
| SEQ | RAVVGKVFLEQARRALNLATNFGTNHRTGCNGCYVTPGKLSIPQDGEKNAAGCTSCL |
| ID | MKATASYVSYPKPLGEKVAKYSTLDALKGFPWYSLRLNLRPNYRGKPINGVQEVAPV |
| NO: | SKFRLAEEVIQAVQRYHFTELEQSFPGGRRRLRELRAFYTKEYRRAPEQRQHVVNGDR |
| 124 | NIVVVTVLHELGFSVGMFNEVELLPKTPIECAVNVFIRGNRVLLEVRKPQFDKERLLVE |
| | SLWKKDSRRHTAKWTPPNNEGRIFTAEGWKDFQLPLLLGSTSRSLRAIEKEGFVQLAP |
| | GRDPDYNNTIDEQHSGRPFLPLYLYLQGTISQEYCVFAGTWVIPFQDGISPYSTKDTFQ |
| | PDLKRKAYSLLLDAVKHRLGNKVASGLQYGRFPAIEELKRLVRMHGATRKIPRGEKD |
| | LLKKGDPDTPEWWLLEQYPEFWRLCDAAAKRVSQNVGLLLSLKKQPLWQRRWLESR |
| | TRNEPLDNLPLSMALTLHLTNEEAL |
| SEQ | AAVYSKFYIENHFKMGIPETLSRIRGPSIIQGFSVNENYINIAGVGDRDFIFGCKKCKYT |
| ID | RGKPSSKKINKCHPCKRSTYPEPVIDVRGSISEFKYKIYNKLKQEPNQSIKQNTKGRMN |
| NO: | PSDHTSSNDGIIINGIDNRIAYNVIFSSYKHLMEKQINLLRDTTKRKARQIKKYNNSGKK |
| 125 | KHSLRSQTKGNLKNRYHMLGMFKKGSLTITNEGDFITAVRKVGLDISLYKNESLNKQE |
| | VETELCLNIKWGRTKSYTVSGYIPLPINIDWKLYLFEKETGLTLRLFGNKYKIQSKKFLI |
| | AQLFKPKRPPCADPVVKKAQKWSALNAHVQQMAGLFSDSHLLKRELKNRMHKQLD |
| | FKSLWVGTEDYIKWFEELSRSYVEGAEKSLEFFRQDYFCFNYTKQTTM |
| SEQ | PQQQRDLMLMAANYDQDYGNGCGPCTVVASAAYRPDPQAQHGCKRHLRTLGASAV |
| ID | THVGLGDRTATITALHRLRGPAALAARARAAQAASAPMTPDTDAPDDRRRLEAIDAD |
| NO: | DVVLVGAHRALWSAVRRWADDRRAALRRRLHSEREWLLKDQIRWAELYTLIEASGT |
| 126 | PPQGRWRNTLGALRGQSRWRRVLAPTMRATCAETHAELWDALAELVPEMAKDRRG |
| | LLRPPVEADALWRAPMIVEGWRGGHSVVVDAVAPPLDLPQPCAWTAVRLSGDPRQR |
| | WGLHLAVPPLGQVQPPDPLKATLAVSMRHRGGVRVRTLQAMAVDADAPMQRHLQV |
| | PLTLQRGGGLQWGIHSRGVRRREARSMASWEGPPIWTGLQLVNRWKGQGSALLAPD |
| | RPPDTPPYAPDAAVAPAQPDTKRARRTLKEACTVCRCAPGHMRQLQVTLTGDGTWR |
| | RFRLRAPQGAKRKAEVLKVATQHDERIANYTAWYLKRPEHAAGCDTCDGDSRLDGA |
| | CRGCRPLLVGDQCFRRYLDKIEADRDDGLAQIKPKAQEAVAAMAAKRDARAQKVAA |
| | RAAKLSEATGQRTAATRDASHEARAQKELEAVATEGTTVRHDAAAVSAFGSWVARK |
| | GDEYRHQVGVLANRLEHGLRLQELMAPDSVVADQQRASGHARVGYRYVLTAM |
| SEQ | AVAHPVGRGNAGSPGARGPEELPRQLVNRASNVTRPATYGCAPCRHVRLSIPKPVLTG |
| ID | CRACEQTTHPAPKRAVRGGADAAKYDLAAFFAGWAADLEGRNRRRQVHAPLDPQP |
| NO: | DPNHEPAVTLQKIDLAEVSIEEFQRVLARSVKHRHDGRASREREKARAYAQVAKKRR |
| 127 | NSHAHGARTRRAVRRQTRAVRRAHRMGANSGEILVASGAEDPVPEAIDHAAQLRRRI |
| | RACARDLEGLRHLSRRYLKTLEKPCRRPRAPDLGRARCHALVESLQAAERELEELRRC |
| | DSPDTAMRRLDAVLAAAASTDATFATGWTVVGMDLGVAPRGSAAPEVSPMEMAISV |
| | FWRKGSRRVIVSKPIAGMPIRRHELIRLEGLGTLRLDGNHYTGAGVTKGRGLSEGTEP |
| | DFREKSPSTLGFTLSDYRHESRWRPYGAKQGKTARQFFAAMSRELRALVEHQVLAPM |
| | GPPLLEAHERRFETLLKGQDNKSIHAGGGGRYVWRGPPDSKKRPAADGDWFRFGRG |
| | HADHRGWANKRHELAANYLQSAFRLWSTLAEAQEPTPYARYKYTRVTM |
| SEQ | WDFLTLQVYERHTSPEVCVAGNSTKCASGTRKSDHTHGVGVKLGAQEINVSANDDR |
| ID | DHEVGCNICVISRVSLDIKGWRYGCESCVQSTPEWRSIVRFDRNHKEAKGECLSRFEY |
| NO: | WGAQSIARSLKRNKLMGGVNLDELAIVQNENVVKTSLKHLFDKRKDRIQANLKAVK |
| 128 | VRMRERRKSGRQRKALRRQCRKLKRYLRSYDPSDIKEGNSCSAFTKLGLDIGISPNKPP |
| | KIEPKVEVVFSLFYQGACDKIVTVSSPESPLPRSWKIKIDGIRALYVKSTKVKFGGRTFR |
| | AGQRNNRRKVRPPNVKKGKRKGSRSQFFNKFAVGLDAVSQQLPIASVQGLWGRAET |
| | KKAQTICLKQLESNKPLKESQRCLFLADNWVVRVCGFLRALSQRQGPTPYIRYRYRCN |
| | M |
| SEQ | ARNVGQRNASRQSKRESAKARSRRVTGGHASVTQGVALINAAANADRDHTTGCEPC |
| ID | TWERVNLPLQEVIHGCDSCTKSSPFWRDIKVVNKGYREAKEEIMRIASGISADHLSRAL |
| NO: | SHNKVMGRLNLDEVCILDFRTVLDTSLKHLTDSRSNGIKEHIRAVHRKIRMRRKSGKT |
| 129 | ARALRKQYFALRRQWKAGHKPNSIREGNSLTALRAVGFDVGVSEGTEPMPAPQTEVV |
| | LSVFYKGSATRILRISSPHPIAKRSWKVKIAGIKALKLIRREHDFSFGRETYNASQRAEK |
| | RKFSPHAARKDFFNSFAVQLDRLAQQLCVSSVENLWVTEPQQKLLTLAKDTAPYGIRE |
| | GARFADTRARLAWNWVFRVCGFTRALHQEQEPTPYCRFTWRSKM |

In some embodiments, the Type V CRISPR/Cas enzyme is a CasΦ nuclease. A CasΦ polypeptide can function as an endonuclease that catalyzes cleavage at a specific sequence in a target nucleic acid. A programmable CasΦ nuclease of the present disclosure may have a single active site in a RuvC domain that is capable of catalyzing pre-crRNA processing and nicking or cleaving of nucleic acids. This compact catalytic site may render the programmable CasΦ nuclease especially advantageous for genome engineering and new functionalities for genome manipulation.

TABLE 3 provides amino acid sequences of illustrative Case polypeptides that can be used in compositions and methods of the disclosure.

TABLE 3
CasΦ Amino Acid Sequences

| | SEQ ID | |
|---|---|---|
| Name | NO | Amino Acid Sequence |
| CasΦ.1 | SEQ ID | MADTPTLFTQFLRHHLPGQRFRKDILKQAGRILANKGEDATIA |
| | NO: 274 | FLRGKSEESPPDFQPPVKCPIIACSRPLTEWPIYQASVAIQGYV |
| | | YGQSLAEFEASDPGCSKDGLLGWFDKTGVCTDYFSVQGLNLI |
| | | FQNARKRYIGVQTKVTNRNEKRHKKLKRINAKRIAEGLPELT |
| | | SDEPESALDETGHLIDPPGLNTNIYCYQQVSPKPLALSEVNQLP |
| | | TAYAGYSTSGDDPIQPMVTKDRLSISKGQPGYIPEHQRALLSQ |
| | | KKHRRMRGYGLKARALLVIVRIQDDWAVIDLRSLLRNAYWR |
| | | RIVQTKEPSTITKLLKLVTGDPVLDATRMVATFTYKPGIVQVR |
| | | SAKCLKNKQGSKLFSERYLNETVSVTSIDLGSNNLVAVATYR |
| | | LVNGNTPELLQRFTLPSHLVKDFERYKQAHDTLEDSIQKTAV |
| | | ASLPQGQQTEIRMWSMYGFREAQERVCQELGLADGSIPWNV |
| | | MTATSTILTDLFLARGGDPKKCMFTSEPKKKKNSKQVLYKIR |
| | | DRAWAKMYRTLLSKETREAWNKALWGLKRGSPDYARLSKR |
| | | KEELARRCVNYTISTAEKRAQCGRTIVALEDLNIGFFHGRGKQ |
| | | EPGWVGLFTRKKENRWLMQALHKAFLELAHHRGYHVIEVNP |
| | | AYTSQTCPVCRHCDPDNRDQHNREAFHCIGCGFRGNADLDV |
| | | ATHNIAMVAITGESLKRARGSVASKTPQPLAAE |
| CasΦ.2 | SEQ ID | MPKPAVESEFSKVLKKHFPGERFRSSYMKRGGKILAAQGEEA |
| | NO: 275 | VVAYLQGKSEEEPPNFQPPAKCHVVTKSRDFAEWPIMKASEA |
| | | IQRYIYALSTTERAACKPGKSSESHAAWFAATGVSNHGYSHV |
| | | QGLNLIFDHTLGRYDGVLKKVQLRNEKARARLESINASRADE |
| | | GLPEIKAEEEEVATNETGHLLQPPGINPSFYVYQTISPQAYRPR |
| | | DEIVLPPEYAGYVRDPNAPIPLGVVRNRCDIQKGCPGYIPEWQ |
| | | REAGTAISPKTGKAVTVPGLSPKKNKRMRRYWRSEKEKAQD |
| | | ALLVTVRIGTDWVVIDVRGLLRNARWRTIAPKDISLNALLDLF |
| | | TGDPVIDVRRNIVTFTYTLDACGTYARKWTLKGKQTKATLD |
| | | KLTATQTVALVAIDLGQTNPISAGISRVTQENGALQCEPLDRF |
| | | TLPDDLLKDISAYRIAWDRNEEELRARSVEALPEAQQAEVRA |
| | | LDGVSKETARTQLCADFGLDPKRLPWDKMSSNTTFISEALLS |
| | | NSVSRDQVFFTPAPKKGAKKKAPVEVMRKDRTWARAYKPRL |
| | | SVEAQKLKNEALWALKRTSPEYLKLSRRKEELCRRSINYVIEK |
| | | TRRRTQCQIVIPVIEDLNVRFFHGSGKRLPGWDNFFTAKKENR |
| | | WFIQGLHKAFSDLRTHRSFYVFEVRPERTSITCPKCGHCEVGN |
| | | RDGEAFQCLSCGKTCNADLDVATHNLTQVALTGKTMPKREE |
| | | PRDAQGTAPARKTKKASKSKAPPAEREDQTPAQEPSQTS |
| CasΦ.3 | SEQ ID | MYILEMADLKSEPSLLAKLLRDRFPGKYWLPKYWKLAEKKR |
| | NO: 276 | LTGGEEAACEYMADKQLDSPPPNFRPPARCVILAKSRPFEDW |
| | | PVHRVASKAQSFVIGLSEQGFAALRAAPPSTADARRDWLRSH |
| | | GASEDDLMALEAQLLETIMGNAISLHGGVLKKIDNANVKAA |
| | | KRLSGRNEARLNKGLQELPPEQEGSAYGADGLLVNPPGLNLN |
| | | IYCRKSCCPKPVKNTARFVGHYPGYLRDSDSILISGTMDRLTII |
| | | EGMPGHIPAWQREQGLVKPGGRRRRLSGSESNMRQKVDPST |
| | | GPRRSTRSGTVNRSNQRTGRNGDPLLVEIRMKEDWVLLDAR |
| | | GLLRNLRWRESKRGLSCDHEDLSLSGLLALFSGDPVIDPVRNE |
| | | VVFLYGEGIIPVRSTKPVGTRQSKKLLERQASMGPLTLISCDL |
| | | GQTNLIAGRASAISLTHGSLGVRSSVRIELDPEIIKSFERLRKDA |
| | | DRLETEILTAAKETLSDEQRGEVNSHEKDSPQTAKASLCRELG |
| | | LHPPSLPWGQMGPSTTFIADMLISHGRDDDAFLSHGEFPTLEK |
| | | RKKFDKRFCLESRPLLSSETRKALNESLWEVKRTSSEYARLSQ |
| | | RKKEMARRAVNFVVEISRRKTGLSNVIVNIEDLNVRIFHGGG |
| | | KQAPGWDGFFRPKSENRWFIQAIHKAFSDLAAHHGIPVIESDP |
| | | QRTSMTCPECGHCDSKNRNGVRFLCKGCGASMDADFDAACR |
| | | NLERVALTGKPMPKPSTSCERLLSATTGKVCSDHSLSHDAIEK |
| | | AS |
| CasΦ.4 | SEQ ID | MEKEITELTKIRREFPNKKFSSTDMKKAGKLLKAEGPDAVRD |
| | NO: 277 | FLNSCQEIIGDFKPPVKTNIVSISRPFEEWPVSMVGRAIQEYYFS |
| | | LTKEELESVHPGTSSEDHKSFFNITGLSNYNYTSVQGLNLIFKN |
| | | AKAIYDGTLVKANNKNKKLEKKFNEINHKRSLEGLPIITPDFE |
| | | EPFDENGHLNNPPGINRNIYGYQGCAAKVFVPSKHKMVSLPK |
| | | EYEGYNRDPNLSLAGFRNRLEIPEGEPGHVPWFQRMDIPEGQI |
| | | GHVNKIQRFNFVHGKNSGKVKFSDKTGRVKRYHHSKYKDAT |
| | | KPYKFLEESKKVSALDSILAIITIGDDWVVFDIRGLYRNVFYRE |
| | | LAQKGLTAVQLLDLFTGDPVIDPKKGVVTFSYKEGVVPVFSQ |
| | | KIVPRFKSRDTLEKLTSQGPVALLSVDLGQNEPVAARVCSLK |
| | | NINDKITLDNSCRISFLDDYKKQIKDYRDSLDELEIKIRLEAINS |
| | | LETNQQVEIRDLDVFSADRAKANTVDMFDIDPNLISWDSMSD |
| | | ARVSTQISDLYLKNGGDESRVYFEINNKRIKRSDYNISQLVRP |
| | | KLSDSTRKNLNDSIWKLKRTSEEYLKLSKRKLELSRAVVNYTI |
| | | RQSKLLSGINDIVIILEDLDVKKKFNGRGIRDIGWDNFFSSRKE |
| | | NRWFIPAFHKAFSELSSNRGLCVIEVNPAWTSATCPDCGFCSK |
| | | ENRDGINFTCRKCGVSYHADIDVATLNIARVAVLGKPMSGPA |
| | | DRERLGDTKKPRVARSRKTMKRKDISNSTVEAMVTA |
| CasΦ.5 | SEQ ID | MDMLDTETNYATETPAQQQDYSPKPPKKAQRAPKGFSKKAR |
| | NO: 278 | PEKKPPKPITLFTQKHFSGVRFLKRVIRDASKILKLSESRTITFL |
| | | EQAIERDGSAPPDVTPPVHNTIMAVTRPFEEWPEVILSKALQK |
| | | HCYALTKKIKIKTWPKKGPGKKCLAAWSARTKIPLIPGQVQA |
| | | TNGLFDRIGSIYDGVEKKVTNRNANKKLEYDEAIKEGRNPAV |
| | | PEYETAYNIDGTLINKPGYNPNLYITQSRTPRLITEADRPLVEKI |
| | | LWQMVEKKTQSRNQARRARLEKAAHLQGLPVPKFVPEKVD |
| | | RSQKIEIRIIDPLDKIEPYMPQDRMAIKASQDGHVPYWQRPFLS |
| | | KRRNRRVRAGWGKQVSSIQAWLTGALLVIVRLGNEAFLADIR |
| | | GALRNAQWRKLLKPDATYQSLFNLFTGDPVVNTRTNHLTMA |
| | | YREGVVNIVKSRSFKGRQTREHLLTLLGQGKTVAGVSFDLGQ |
| | | KHAAGLLAAHFGLGEDGNPVFTPIQACFLPQRYLDSLTNYRN |
| | | RYDALTLDMRRQSLLALTPAQQQEFADAQRDPGGQAKRACC |
| | | LKLNLNPDEIRWDLVSGISTMISDLYIERGGDPRDVHQQVETK |
| | | PKGKRKSEIRILKIRDGKWAYDFRPKIADETRKAQREQLWKL |
| | | QKASSEFERLSRYKINIARAIANWALQWGRELSGCDIVIPVLE |
| | | DLNVGSKFFDGKGKWLLGWDNRFTPKKENRWFIKVLHKAV |
| | | AELAPHRGVPVYEVMPHRTSMTCPACHYCHPTNREGDRFEC |
| | | QSCHVVKNTDRDVAPYNILRVAVEGKTLDRWQAEKKPQAEP |
| | | DRPMILIDNQES |
| CasΦ.6 | SEQ ID | MDMLDTETNYATETPAQQQDYSPKPPKKAQRAPKGFSKKAR |
| | NO: 279 | PEKKPPKPITLFTQKHFSGVRFLKRVIRDASKILKLSESRTITFL |
| | | EQAIERDGSAPPDVTPPVHNTIMAVTRPFEEWPEVILSKALQK |
| | | HCYALTKKIKIKTWPKKGPGKKCLAAWSARTKIPLIPGQVQA |
| | | TNGLFDRIGSIYDGVEKKVTNRNANKKLEYDEAIKEGRNPAV |
| | | PEYETAYNIDGTLINKPGYNPNLYITQSRTPRLITEADRPLVEKI |
| | | LWQMVEKKTQSRNQARRARLEKAAHLQGLPVPKFVPEKVD |
| | | RSQKIEIRIIDPLDKIEPYMPQDRMAIKASQDGHVPYWQRPFLS |
| | | KRRNRRVRAGWGKQVSSIQAWLTGALLVIVRLGNEAFLADIR |
| | | GALRNAQWRKLLKPDATYQSLFNLFTGDPVVNTRTNHLTMA |
| | | YREGVVDIVKSRSFKGRQTREHLLTLLGQGKTVAGVSFDLGQ |
| | | KHAAGLLAAHFGLGEDGNPVFTPIQACFLPQRYLDSLTNYRN |
| | | RYDALTLDMRRQSLLALTPAQQQEFADAQRDPGGQAKRACC |
| | | LKLNLNPDEIRWDLVSGISTMISDLYIERGGDPRDVHQQVETK |
| | | PKGKRKSEIRILKIRDGKWAYDFRPKIADETRKAQREQLWKL |
| | | QKASSEFERLSRYKINIARAIANWALQWGRELSGCDIVIPVLE |
| | | DLNVGSKFFDGKGKWLLGWDNRFTPKKENRWFIKVLHKAV |
| | | AELAPHKGVPVYEVMPHRTSMTCPACHYCHPTNREGDRFEC |
| | | QSCHVVKNTDRDVAPYNILRVAVEGKTLDRWQAEKKPQAEP |
| | | DRPMILIDNQES |
| CasΦ.7 | SEQ ID | MSSLPTPLELLKQKHADLFKGLQFSSKDNKMAGKVLKKDGE |
| | NO: 280 | EAALAFLSERGVSRGELPNFRPPAKTLVVAQSRPFEEFPIYRVS |
| | | EAIQLYVYSLSVKELETVPSGSSTKKEHQRFFQDSSVPDFGYT |
| | | SVQGLNKIFGLARGIYLGVITRGENQLQKAKSKHEALNKKRR |
| | | ASGEAETEFDPTPYEYMTPERKLAKPPGVNHSIMCYVDISVDE |
| | | FDFRNPDGIVLPSEYAGYCREINTAIEKGTVDRLGHLKGGPGY |
| | | IPGHQRKESTTEGPKINFRKGRIRRSYTALYAKRDSRRVRQGK |
| | | LALPSYRHHMMRLNSNAESAILAVIFFGKDWVVFDLRGLLRN |
| | | VRWRNLFVDGSTPSTLLGMFGDPVIDPKRGVVAFCYKEQIVP |
| | | VVSKSITKMVKAPELLNKLYLKSEDPLVLVAIDLGQTNPVGV |
| | | GVYRVMNASLDYEVVTRFALESELLREIESYRQRTNAFEAQIR |
| | | AETFDAMTSEEQEEITRVRAFSASKAKENVCHRFGMPVDAVD |
| | | WATMGSNTIHIAKWVMRHGDPSLVEVLEYRKDNEIKLDKNG |
| | | VPKKVKLTDKRIANLTSIRLRFSQETSKHYNDTMWELRRKHP |
| | | VYQKLSKSKADFSRRVVNSIIRRVNHLVPRARIVFIIEDLKNLG |
| | | KVFHGSGKRELGWDSYFEPKSENRWFIQVLHKAFSETGKHK |
| | | GYYIIECWPNWTSCTCPKCSCCDSENRHGEVFRCLACGYTCN |
| | | TDFGTAPDNLVKIATTGKGLPGPKKRCKGSSKGKNPKIARSSE |
| | | TGVSVTESGAPKVKKSSPTQTSQSSSQSAP |
| CasΦ.8 | SEQ ID | MNKIEKEKTPLAKLMNENFAGLRFPFAIIKQAGKKLLKEGEL |
| | NO: 281 | KTIEYMTGKGSIEPLPNFKPPVKCLIVAKRRDLKYFPICKASCE |
| | | IQSYVYSLNYKDFMDYFSTPMTSQKQHEEFFKKSGLNIEYQN |
| | | VAGLNLIFNNVKNTYNGVILKVKNRNEKLKKKAIKNNYEFEE |
| | | IKTFNDDGCLINKPGINNVIYCFQSISPKILKNITHLPKEYNDYD |
| | | CSVDRNIIQKYVSRLDIPESQPGHVPEWQRKLPEFNNTNNPRR |
| | | RRKWYSNGRNISKGYSVDQVNQAKIEDSLLAQIKIGEDWIILD |
| | | IRGLLRDLNRRELISYKNKLTIKDVLGFFSDYPIIDIKKNLVTFC |
| | | YKEGVIQVVSQKSIGNKKSKQLLEKLIENKPIALVSIDLGQTNP |
| | | VSVKISKLNKINNKISIESFTYRFLNEEILKEIEKYRKDYDKLEL |
| | | KLINEA |
| CasΦ.9 | SEQ ID | MDMLDTETNYATETPSQQQDYSPKPPKKDRRAPKGFSKKAR |
| | NO: 282 | PEKKPPKPITLFTQKHFSGVRFLKRVIRDASKILKLSESRTITFL |
| | | EQAIERDGSAPPDVTPPVHNTIMAVTRPFEEWPEVILSKALQK |
| | | HCYALTKKIKIKTWPKKGPGKKCLAAWSARTKIPLIPGQVQA |
| | | TNGLFDRIGSIYDGVEKKVTNRNANKKLEYDEAIKEGRNPAV |
| | | PEYETAYNIDGTLINKPGYNPNLYITQSRTPRLITEADRPLVEKI |
| | | LWQMVEKKTQSRNQARRARLEKAAHLQGLPVPKFVPEKVD |
| | | RSQKIEIRIIDPLDKIEPYMPQDRMAIKASQDGHVPYWQRPFLS |
| | | KRRNRRVRAGWGKQVSSIQAWLTGALLVIVRLGNEAFLADIR |
| | | GALRNAQWRKLLKPDATYQSLFNLFTGDPVVNTRTNHLTMA |
| | | YREGVVDIVKSRSFKGRQTREHLLTLLGQGKTVAGVSFDLGQ |
| | | KHAAGLLAAHFGLGEDGNPVFTPIQACFLPQRYLDSLTNYRN |
| | | RYDALTLDMRRQSLLALTPAQQQEFADAQRDPGGQAKRACC |
| | | LKLNLNPDEIRWDLVSGISTMISDLYIERGGDPRDVHQQVETK |
| | | PKGKRKSEIRILKIRDGKWAYDFRPKIADETRKAQREQLWKL |
| | | QKASSEFERLSRYKINIARAIANWALQWGRELSGCDIVIPVLE |
| | | DLNVGSKFFDGKGKWLLGWDNRFTPKKENRWFIKVLHKAV |
| | | AELAPHRGVPVYEVMPHRTSMTCPACHYCHPTNREGDRFEC |
| | | QSCHVVKNTDRDVAPYNILRVAVEGKTLDRWQAEKKPQAEP |
| | | DRPMILIDNQES |
| CasΦ.10 | SEQ ID | MDMLDTETNYATETPSQQQDYSPKPPKKDRRAPKGFSKKAR |
| | NO: 283 | PEKKPPKPITLFTQKHFSGVRFLKRVIRDASKILKLSESRTITFL |
| | | EQAIERDGSAPPDVTPPVHNTIMAVTRPFEEWPEVILSKALQK |
| | | HCYALTKKIKIKTWPKKGPGKKCLAAWSARTKIPLIPGQVQA |
| | | TNGLFDRIGSIYDGVEKKVTNRNANKKLEYDEAIKEGRNPAV |
| | | PEYETAYNIDGTLINKPGYNPNLYITQSRTPRLITEADRPLVEKI |
| | | LWQMVEKKTQSRNQARRARLEKAAHLQGLPVPKFVPEKVD |
| | | RSQKIEIRIIDPLDKIEPYMPQDRMAIKASQDGHVPYWQRPFLS |
| | | KRRNRRVRAGWGKQVSSIQAWLTGALLVIVRLGNEAFLADIR |
| | | GALRNAQWRKLLKPDATYQSLFNLFTGDPVVNTRTNHLTMA |
| | | YREGVVNIVKSRSFKGRQTREHLLTLLGQGKTVAGVSFDLGQ |
| | | KHAAGLLAAHFGLGEDGNPVFTPIQACFLPQRYLDSLTNYRN |
| | | RYDALTLDMRRQSLLALTPAQQQEFADAQRDPGGQAKRACC |
| | | LKLNLNPDEIRWDLVSGISTMISDLYIERGGDPRDVHQQVETK |
| | | PKGKRKSEIRILKIRDGKWAYDFRPKIADETRKAQREQLWKL |
| | | QKASSEFERLSRYKINIARAIANWALQWGRELSGCDIVIPVLE |
| | | DLNVGSKFFDGKGKWLLGWDNRFTPKKENRWFIKVLHKAV |
| | | AELAPHRGVPVYEVMPHRTSMTCPACHYCHPTNREGDRFEC |
| | | QSCHVVKNTDRDVAPYNILRVAVEGKTLDRWQAEKKPQAEP |
| | | DRPMILIDNQES |
| CasΦ.11 | SEQ ID | MSNKTTPPSPLSLLLRAHFPGLKFESQDYKIAGKKLRDGGPEA |
| | NO: 284 | VISYLTGKGQAKLKDVKPPAKAFVIAQSRPFIEWDLVRVSRQI |
| | | QEKIFGIPATKGRPKQDGLSETAFNEAVASLEVDGKSKLNEET |
| | | RAAFYEVLGLDAPSLHAQAQNALIKSAISIREGVLKKVENRNE |
| | | KNLSKTKRRKEAGEEATFVEEKAHDERGYLIHPPGVNQTIPG |
| | | YQAVVIKSCPSDFIGLPSGCLAKESAEALTDYLPHDRMTIPKG |
| | | QPGYVPEWQHPLLNRRKNRRRRDWYSASLNKPKATCSKRSG |
| | | TPNRKNSRTDQIQSGRFKGAIPVLMRFQDEWVIIDIRGLLRNA |
| | | RYRKLLKEKSTIPDLLSLFTGDPSIDMRQGVCTFIYKAGQACS |
| | | AKMVKTKNAPEILSELTKSGPVVLVSIDLGQTNPIAAKVSRVT |
| | | QLSDGQLSHETLLRELLSNDSSDGKEIARYRVASDRLRDKLA |
| | | NLAVERLSPEHKSEILRAKNDTPALCKARVCAALGLNPEMIA |
| | | WDKMTPYTEFLATAYLEKGGDRKVATLKPKNRPEMLRRDIK |
| | | FKGTEGVRIEVSPEAAEAYREAQWDLQRTSPEYLRLSTWKQE |
| | | LTKRILNQLRHKAAKSSQCEVVVMAFEDLNIKMMHGNGKW |
| | | ADGGWDAFFIKKRENRWFMQAFHKSLTELGAHKGVPTIEVT |
| | | PHRTSITCTKCGHCDKANRDGERFACQKCGFVAHADLEIATD |
| | | NIERVALTGKPMPKPESERSGDAKKSVGARKAAFKPEEDAEA |
| | | AE |
| CasΦ.12 | SEQ ID | MIKPTVSQFLTPGFKLIRNHSRTAGLKLKNEGEEACKKFVREN |
| | NO: 285 | EIPKDECPNFQGGPAIANIIAKSREFTEWEIYQSSLAIQEVIFTLP |
| | | KDKLPEPILKEEWRAQWLSEHGLDTVPYKEAAGLNLIIKNAV |
| | | NTYKGVQVKVDNKNKNNLAKINRKNEIAKLNGEQEISFEEIK |
| | | AFDDKGYLLQKPSPNKSIYCYQSVSPKPFITSKYHNVNLPEEYI |
| | | GYYRKSNEPIVSPYQFDRLRIPIGEPGYVPKWQYTFLSKKENK |
| | | RRKLSKRIKNVSPILGIICIKKDWCVFDMRGLLRTNHWKKYH |
| | | KPTDSINDLFDYFTGDPVIDTKANVVRFRYKMENGIVNYKPV |
| | | REKKGKELLENICDQNGSCKLATVDVGQNNPVAIGLFELKKV |
| | | NGELTKTLISRHPTPIDFCNKITAYRERYDKLESSIKLDAIKQLT |
| | | SEQKIEVDNYNNNFTPQNTKQIVCSKLNINPNDLPWDKMISGT |
| | | HFISEKAQVSNKSEIYFTSTDKGKTKDVMKSDYKWFQDYKPK |
| | | LSKEVRDALSDIEWRLRRESLEFNKLSKSREQDARQLANWISS |
| | | MCDVIGIENLVKKNNFFGGSGKREPGWDNFYKPKKENRWWI |
| | | NAIHKALTELSQNKGKRVILLPAMRTSITCPKCKYCDSKNRN |
| | | GEKFNCLKCGIELNADIDVATENLATVAITAQSMPKPTCERSG |
| | | DAKKPVRARKAKAPEFHDKLAPSYTVVLREAV |
| CasΦ.13 | SEQ ID | MRQPAEKTAFQVFRQEVIGTQKLSGGDAKTAGRLYKQGKME |
| | NO: 286 | AAREWLLKGARDDVPPNFQPPAKCLVVAVSHPFEEWDISKTN |
| | | HDVQAYIYAQPLQAEGHLNGLSEKWEDTSADQHKLWFEKTG |
| | | VPDRGLPVQAINKIAKAAVNRAFGVVRKVENRNEKRRSRDN |
| | | RIAEHNRENGLTEVVREAPEVATNADGFLLHPPGIDPSILSYAS |
| | | VSPVPYNSSKHSFVRLPEEYQAYNVEPDAPIPQFVVEDRFAIPP |
| | | GQPGYVPEWQRLKCSTNKHRRMRQWSNQDYKPKAGRRAKP |
| | | LEFQAHLTRERAKGALLVVMRIKEDWVVFDVRGLLRNVEWR |
| | | KVLSEEAREKLTLKGLLDLFTGDPVIDTKRGIVTFLYKAEITKI |
| | | LSKRTVKTKNARDLLLRLTEPGEDGLRREVGLVAVDLGQTHP |
| | | IAAAIYRIGRTSAGALESTVLHRQGLREDQKEKLKEYRKRHT |
| | | ALDSRLRKEAFETLSVEQQKEIVTVSGSGAQITKDKVCNYLG |
| | | VDPSTLPWEKMGSYTHFISDDFLRRGGDPNIVHFDRQPKKGK |
| | | VSKKSQRIKRSDSQWVGRMRPRLSQETAKARMEADWAAQN |
| | | ENEEYKRLARSKQELARWCVNTLLQNTRCITQCDEIVVVIED |
| | | LNVKSLHGKGAREPGWDNFFTPKTENRWFIQILHKTFSELPK |
| | | HRGEHVIEGCPLRTSITCPACSYCDKNSRNGEKFVCVACGATF |
| | | HADFEVATYNLVRLATTGMPMPKSLERQGGGEKAGGARKA |
| | | RKKAKQVEKIVVQANANVTMNGASLHSP |
| CasΦ.14 | SEQ ID | MSSLPTPLELLKQKHADLFKGLQFSSKDNKMAGKVLKKDGE |
| | NO: 287 | EAALAFLSERGVSRGELPNFRPPAKTLVVAQSRPFEEFPIYRVS |
| | | EAIQLYVYSLSVKELETVPSGSSTKKEHQRFFQDSSVPDFGYT |
| | | SVQGLNKIFGLARGIYLGVITRGENQLQKAKSKHEALNKKRR |
| | | ASGEAETEFDPTPYEYMTPERKLAKPPGVNHSIMCYVDISVDE |
| | | FDFRNPDGIVLPSEYAGYCREINTAIEKGTVDRLGHLKGGPGY |
| | | IPGHQRKESTTEGPKINFRKGRIRRSYTALYAKRDSRRVRQGK |
| | | LALPSYRHHMMRLNSNAESAILAVIFFGKDWVVFDLRGLLRN |
| | | VRWRNLFVDGSTPSTLLGMFGDPVIDPKRGVVAFCYKEQIVP |
| | | VVSKSITKMVKAPELLNKLYLKSEDPLVLVAIDLGQTNPVGV |
| | | GVYRVMNASLDYEVVTRFALESELLREIESYRQRTNAFEAQIR |
| | | AETFDAMTSEEQEEITRVRAFSASKAKENVCHRFGMPVDAVD |
| | | WATMGSNTIHIAKWVMRHGDPSLVEVLEYRKDNEIKLDKNG |
| | | VPKKVKLTDKRIANLTSIRLRFSQETSKHYNDTMWELRRKHP |
| | | VYQKLSKSKADFSRRVVNSIIRRVNHLVPRARIVFIIEDLKNLG |
| | | KVFHGSGKRELGWDSYFEPKSENRWFIQVLHKAFSETGKHK |
| | | GYYIIECWPNWTSCTCPKCSCCDSENRHGEVFRCLACGYTCN |
| | | TDFGTAPDNLVKIATTGKGLPGPKKRCKGSSKGKNPKIARSSE |
| | | TGVSVTESGAPKVKKSSPTQTSQSSSQSAP |
| CasΦ.15 | SEQ ID | MIKPTVSQFLTPGFKLIRNHSRTAGLKLKNEGEEACKKFVREN |
| | NO: 288 | EIPKDECPNFQGGPAIANIIAKSREFTEWEIYQSSLAIQEVIFTLP |
| | | KDKLPEPILKEEWRAQWLSEHGLDTVPYKEAAGLNLIIKNAV |
| | | NTYKGVQVKVDNKNKNNLAKINRKNEIAKLNGEQEISFEEIK |
| | | AFDDKGYLLQKPSPNKSIYCYQSVSPKPFITSKYHNVNLPEEYI |
| | | GYYRKSNEPIVSPYQFDRLRIPIGEPGYVPKWQYTFLSKKENK |
| | | RRKLSKRIKNVSPILGIICIKKDWCVFDMRGLLRTNHWKKYH |
| | | KPTDSINDLFDYFTGDPVIDTKANVVRFRYKMENGIVNYKPV |
| | | REKKGKELLENICDQNGSCKLATVDVGQNNPVAIGLFELKKV |
| | | NGELTKTLISRHPTPIDFCNKITAYRERYDKLESSIKLDAIKQLT |
| | | SEQKIEVDNYNNNFTPQNTKQIVCSKLNINPNDLPWDKMISGT |
| | | HFISEKAQVSNKSEIYFTSTDKGKTKDVMKSDYKWFQDYKPK |
| | | LSKEVRDALSDIEWRLRRESLEFNKLSKSREQDARQLANWISS |
| | | MCDVIGIENLVKKNNFFGGSGKREPGWDNFYKPKKENRWWI |
| | | NAIHKALTELSQNKGKRVILLPAMRTSITCPKCKYCDSKNRN |
| | | GEKFNCLKCGIELNADIDVATENLATVAITAQSMPKPTCERSG |
| | | DAKKPVRARKAKAPEFHDKLAPSYTVVLREAV |
| CasΦ.16 | SEQ ID | MSNKTTPPSPLSLLLRAHFPGLKFESQDYKIAGKKLRDGGPEA |
| | NO: 289 | VISYLTGKGQAKLKDVKPPAKAFVIAQSRPFIEWDLVRVSRQI |
| | | QEKIFGIPATKGRPKQDGLSETAFNEAVASLEVDGKSKLNEET |
| | | RAAFYEVLGLDAPSLHAQAQNALIKSAISIREGVLKKVENRNE |
| | | KNLSKTKRRKEAGEEATFVEEKAHDERGYLIHPPGVNQTIPG |
| | | YQAVVIKSCPSDFIGLPSGCLAKESAEALTDYLPHDRMTIPKG |
| | | QPGYVPEWQHPLLNRRKNRRRRDWYSASLNKPKATCSKRSG |
| | | TPNRKNSRTDQIQSGRFKGAIPVLMRFQDEWVIIDIRGLLRNA |
| | | RYRKLLKEKSTIPDLLSLFTGDPSIDMRQGVCTFIYKAGQACS |
| | | AKMVKTKNAPEILSELTKSGPVVLVSIDLGQTNPIAAKVSRVT |
| | | QLSDGQLSHETLLRELLSNDSSDGKEIARYRVASDRLRDKLA |
| | | NLAVERLSPEHKSEILRAKNDTPALCKARVCAALGLNPEMIA |
| | | WDKMTPYTEFLATAYLEKGGDRKVATLKPKNRPEMLRRDIK |
| | | FKGTEGVRIEVSPEAAEAYREAQWDLQRTSPEYLRLSTWKQE |
| | | LTKRILNQLRHKAAKSSQCEVVVMAFEDLNIKMMHGNGKW |
| | | ADGGWDAFFIKKRENRWFMQAFHKSLTELGAHKGVPTIEVT |
| | | PHRTSITCTKCGHCDKANRDGERFACQKCGFVAHADLEIATD |
| | | NIERVALTGKPMPKPESERSGDAKKSVGARKAAFKPEEDAEA |
| | | AE |
| CasΦ.17 | SEQ ID | MYSLEMADLKSEPSLLAKLLRDRFPGKYWLPKYWKLAEKKR |
| | NO: 290 | LTGGEEAACEYMADKQLDSPPPNFRPPARCVILAKSRPFEDW |
| | | PVHRVASKAQSFVIGLSEQGFAALRAAPPSTADARRDWLRSH |
| | | GASEDDLMALEAQLLETIMGNAISLHGGVLKKIDNANVKAA |
| | | KRLSGRNEARLNKGLQELPPEQEGSAYGADGLLVNPPGLNLN |
| | | IYCRKSCCPKPVKNTARFVGHYPGYLRDSDSILISGTMDRLTII |
| | | EGMPGHIPAWQREQGLVKPGGRRRRLSGSESNMRQKVDPST |
| | | GPRRSTRSGTVNRSNQRTGRNGDPLLVEIRMKEDWVLLDAR |
| | | GLLRNLRWRESKRGLSCDHEDLSLSGLLALFSGDPVIDPVRNE |
| | | VVFLYGEGIIPVRSTKPVGTRQSKKLLERQASMGPLTLISCDL |
| | | GQTNLIAGRASAISLTHGSLGVRSSVRIELDPEIIKSFERLRKDA |
| | | DRLETEILTAAKETLSDEQRGEVNSHEKDSPQTAKASLCRELG |
| | | LHPPSLPWGQMGPSTTFIADMLISHGRDDDAFLSHGEFPTLEK |
| | | RKKFDKRFCLESRPLLSSETRKALNESLWEVKRTSSEYARLSQ |
| | | RKKEMARRAVNFVVEISRRKTGLSNVIVNIEDLNVRIFHGGG |
| | | KQAPGWDGFFRPKSENRWFIQAIHKAFSDLAAHHGIPVIESDP |
| | | QRTSMTCPECGHCDSKNRNGVRFLCKGCGASMDADFDAACR |
| | | NLERVALTGKPMPKPSTSCERLLSATTGKVCSDHSLSHDAIEK |
| | | AS |
| CasΦ.18 | SEQ ID | MEKEITELTKIRREFPNKKFSSTDMKKAGKLLKAEGPDAVRD |
| | NO: 291 | FLNSCQEIIGDFKPPVKTNIVSISRPFEEWPVSMVGRAIQEYYFS |
| | | LTKEELESVHPGTSSEDHKSFFNITGLSNYNYTSVQGLNLIFKN |
| | | AKAIYDGTLVKANNKNKKLEKKFNEINHKRSLEGLPIITPDFE |
| | | EPFDENGHLNNPPGINRNIYGYQGCAAKVFVPSKHKMVSLPK |
| | | EYEGYNRDPNLSLAGFRNRLEIPEGEPGHVPWFQRMDIPEGQI |
| | | GHVNKIQRFNFVHGKNSGKVKFSDKTGRVKRYHHSKYKDAT |
| | | KPYKFLEESKKVSALDSILAIITIGDDWVVFDIRGLYRNVFYRE |
| | | LAQKGLTAVQLLDLFTGDPVIDPKKGVVTFSYKEGVVPVFSQ |
| | | KIVPRFKSRDTLEKLTSQGPVALLSVDLGQNEPVAARVCSLK |
| | | NINDKITLDNSCRISFLDDYKKQIKDYRDSLDELEIKIRLEAINS |
| | | LETNQQVEIRDLDVFSADRAKANTVDMFDIDPNLISWDSMSD |
| | | ARVSTQISDLYLKNGGDESRVYFEINNKRIKRSDYNISQLVRP |
| | | KLSDSTRKNLNDSIWKLKRTSEEYLKLSKRKLELSRAVVNYTI |
| | | RQSKLLSGINDIVIILEDLDVKKKFNGRGIRDIGWDNFFSSRKE |
| | | NRWFIPAFHKTFSELSSNRGLCVIEVNPAWTSATCPDCGFCSK |
| | | ENRDGINFTCRKCGVSYHADIDVATLNIARVAVLGKPMSGPA |
| | | DRERLGDTKKPRVARSRKTMKRKDISNSTVEAMVTA |
| CasΦ.19 | SEQ ID | MLVRTSTLVQDNKNSRSASRAFLKKPKMPKNKHIKEPTELAK |
| | NO: 292 | LIRELFPGQRFTRAINTQAGKILKHKGRDEVVEFLKNKGIDKE |
| | | QFMDFRPPTKARIVATSGAIEEFSYLRVSMAIQECCFGKYKFP |
| | | KEKVNGKLVLETVGLTKEELDDFLPKKYYENKKSRDRFFLKT |
| | | GICDYGYTYAQGLNEIFRNTRAIYEGVFTKVNNRNEKRREKK |
| | | DKYNEERRSKGLSEEPYDEDESATDESGHLINPPGVNLNIWTC |
| | | EGFCKGPYVTKLSGTPGYEVILPKVFDGYNRDPNEIISCGITDR |
| | | FAIPEGEPGHIPWHQRLEIPEGQPGYVPGHQRFADTGQNNSGK |
| | | ANPNKKGRMRKYYGHGTKYTQPGEYQEVFRKGHREGNKRR |
| | | YWEEDFRSEAHDCILYVIHIGDDWVVCDLRGPLRDAYRRGLV |
| | | PKEGITTQELCNLFSGDPVIDPKHGVVTFCYKNGLVRAQKTIS |
| | | AGKKSRELLGALTSQGPIALIGVDLGQTEPVGARAFIVNQARG |
| | | SLSLPTLKGSFLLTAENSSSWNVFKGEIKAYREAIDDLAIRLKK |
| | | EAVATLSVEQQTEIESYEAFSAEDAKQLACEKFGVDSSFILWE |
| | | DMTPYHTGPATYYFAKQFLKKNGGNKSLIEYIPYQKKKSKKT |
| | | PKAVLRSDYNIACCVRPKLLPETRKALNEAIRIVQKNSDEYQR |
| | | LSKRKLEFCRRVVNYLVRKAKKLTGLERVIIAIEDLKSLEKFF |
| | | TGSGKRDNGWSNFFRPKKENRWFIPAFHKAFSELAPNRGFYV |
| | | IECNPARTSITDPDCGYCDGDNRDGIKFECKKCGAKHHTDLD |
| | | VAPLNIAIVAVTGRPMPKTVSNKSKRERSGGEKSVGASRKRN |
| | | HRKSKANQEMLDATSSAAE |
| CasΦ.20 | SEQ ID | MPKIKKPTEISLLRKEVFPDLHFAKDRMRAASLVLKNEGREA |
| | NO: 293 | AIEYLRVNHEDKPPNFMPPAKTPYVALSRPLEQWPIAQASIAI |
| | | QKYIFGLTKDEFSATKKLLYGDKSTPNTESRKRWFEVTGVPN |
| | | FGYMSAQGLNAIFSGALARYEGVVQKVENRNKKRFEKLSEK |
| | | NQLLIEEGQPVKDYVPDTAYHTPETLQKLAENNHVRVEDLGD |
| | | MIDRLVHPPGIHRSIYGYQQVPPFAYDPDNPKGIILPKAYAGY |
| | | TRKPHDIIEAMPNRLNIPEGQAGYIPEHQRDKLKKGGRVKRLR |
| | | TTRVRVDATETVRAKAEALNAEKARLRGKEAILAVFQIEEDW |
| | | ALIDMRGLLRNVYMRKLIAAGELTPTTLLGYFTETLTLDPRRT |
| | | EATFCYHLRSEGALHAEYVRHGKNTRELLLDLTKDNEKIALV |
| | | TIDLGQRNPLAAAIFRVGRDASGDLTENSLEPVSRMLLPQAYL |
| | | DQIKAYRDAYDSFRQNIWDTALASLTPEQQRQILAYEAYTPD |
| | | DSKENVLRLLLGGNVMPDDLPWEDMTKNTHYISDRYLADGG |
| | | DPSKVWFVPGPRKRKKNAPPLKKPPKPRELVKRSDHNISHLSE |
| | | FRPQLLKETRDAFEKAKIDTERGHVGYQKLSTRKDQLCKEIL |
| | | NWLEAEAVRLTRCKTMVLGLEDLNGPFFNQGKGKVRGWVS |
| | | FFRQKQENRWIVNGFRKNALARAHDKGKYILELWPSWTSQT |
| | | CPKCKHVHADNRHGDDFVCLQCGARLHADAEVATWNLAVV |
| | | AIQGHSLPGPVREKSNDRKKSGSARKSKKANESGKVVGAWA |
| | | AQATPKRATSKKETGTARNPVYNPLETQASCPAP |
| CasΦ.21 | SEQ ID | MTPSPQIARLVETPLAAALKAHHPGKKFRSDYLKKAGKILKD |
| | NO: 294 | QGVEAAMAHLDGKDQAEPPNFKPPAKCRIVARSREFSEWPIV |
| | | KASVEIQKYIYGLTLEERKACDPGKSSASHKAWFAKTGVNTF |
| | | GYSSVQGFNLIFGHTLGRYDGVLVKTENLNKKRAEKNERFRA |
| | | KALAEGRAEPVCPPLVTATNDTGQDVTLEDGRVVRPGQLLQP |
| | | PGINPNIYAYQQVSPKAYVPGIIELPEEFQGYSRDPNAVILPLV |
| | | PRDRLSIPKGQPGYVPEPHREGLTGRKDRRMRRYYETERGTK |
| | | LKRPPLTAKGRADKANEALLVVVRIDSDWVVMDVRGLLRNA |
| | | RWRRLVSKEGITLNGLLDLFTGDPVLNPKDCSVSRDTGDPVN |
| | | DPRHGVVTFCYKLGVVDVCSKDRPIKGFRTKEVLERLTSSGT |
| | | VGMVSIDLGQTNPVAAAVSRVTKGLQAETLETFTLPDDLLGK |
| | | VRAYRAKTDRMEEGFRRNALRKLTAEQQAEITRYNDATEQQ |
| | | AKALVCSTYGIGPEEVPWERMTSNTTYISDHILDHGGDPDTVF |
| | | FMATKRGQNKPTLHKRKDKAWGQKFRPAISVETRLARQAAE |
| | | WELRRASLEFQKLSVWKTELCRQAVNYVMERTKKRTQCDVI |
| | | IPVIEDLPVPLFHGSGKRDPGWANFFVHKRENRWFIDGLHKAF |
| | | SELGKHRGIYVFEVCPQRTSITCPKCGHCDPDNRDGEKFVCLS |
| | | CQATLNADLDVATTNLVRVALTGKVMPRSERSGDAQTPGPA |
| | | RKARTGKIKGSKPTSAPQGATQTDAKAHLSQTGV |
| CasΦ.22 | SEQ ID | MTPSPQIARLVETPLAAALKAHHPGKKFRSDYLKKAGKILKD |
| | NO: 295 | QGVEAAMAHLDGKDQAEPPNFKPPAKCRIVARSREFSEWPIV |
| | | KASVEIQKYIYGLTLEERKACDPGKSSASHKAWFAKTGVNTF |
| | | GYSSVQGFNLIFGHTLGRYDGVLVKTENLNKKRAEKNERFRA |
| | | KALAEGRAEPVCPPLVTATNDTGQDVTLEDGRVVRPGQLLQP |
| | | PGINPNIYAYQQVSPKAYVPGIIELPEEFQGYSRDPNAVILPLV |
| | | PRDRLSIPKGQPGYVPEPHREGLTGRKDRRMRRYYETERGTK |
| | | LKRPPLTAKGRADKANEALLVVVRIDSDWVVMDVRGLLRNA |
| | | RWRRLVSKEGITLNGLLDLFTGDPVLNPKDCSVSRDTGDPVN |
| | | DPRHGVVTFCYKLGVVDVCSKDRPIKGFRTKEVLERLTSSGT |
| | | VGMVSIDLGQTNPVAAAVSRVTKGLQAETLETFTLPDDLLGK |
| | | VRAYRAKTDRMEEGFRRNALRKLTAEQQAEITRYNDATEQQ |
| | | AKALVCSTYGIGPEEVPWERMTSNTTYISDHILDHGGDPDTVF |
| | | FMATKRGQNKPTLHKRKDKAWGQKFRPAISVETRLARQAAE |
| | | WELRRASLEFQKLSVWKTELCRQAVNYVMERTKKRTQCDVI |
| | | IPVIEDLPVPLFHGSGKRDPGWANFFVHKRENRWFIDGLHKAF |
| | | SELGKHRGIYVFEVCPQRTSITCPKCGHCDPDNRDGEKFVCLS |
| | | CQATLHADLDVATTNLVRVALTGKVMPRSERSGDAQTPGPA |
| | | RKARTGKIKGSKPTSAPQGATQTDAKAHLSQTGV |
| CasΦ.23 | SEQ ID | MKTEKPKTALTLLREEVFPGKKYRLDVLKEAGKKLSTKGRE |
| | NO: 296 | ATIEFLTGKDEERPQNFQPPAKTSIVAQSRPFDQWPIVQVSLA |
| | | VQKYIYGLTQSEFEANKKALYGETGKAISTESRRAWFEATGV |
| | | DNFGFTAAQGINPIFSQAVARYEGVIKKVENRNEKKLKKLTK |
| | | KNLLRLESGEEIEDFEPEATFNEEGRLLQPPGANPNIYCYQQIS |
| | | PRIYDPSDPKGVILPQIYAGYDRKPEDIISAGVPNRLAIPEGQPG |
| | | YIPEHQRAGLKTQGRIRCRASVEAKARAAILAVVHLGEDWVV |
| | | LDLRGLLRNVYWRKLASPGTLTLKGLLDFFTGGPVLDARRGI |
| | | ATFSYTLKSAAAVHAENTYKGKGTREVLLKLTENNSVALVT |
| | | VDLGQRNPLAAMIARVSRTSQGDLTYPESVEPLTRLFLPDPFL |
| | | EEVRKYRSSYDALRLSIREAAIASLTPEQQAEIRYIEKFSAGDA |
| | | KKNVAEVFGIDPTQLPWDAMTPRTTYISDLFLRMGGDRSRVF |
| | | FEVPPKKAKKAPKKPPKKPAGPRIVKRTDGMIARLREIRPRLS |
| | | AETNKAFQEARWEGERSNVAFQKLSVRRKQFARTVVNHLVQ |
| | | TAQKMSRCDTVVLGIEDLNVPFFHGRGKYQPGWEGFFRQKK |
| | | ENRWLINDMHKALSERGPHRGGYVLELTPFWTSLRCPKCGH |
| | | TDSANRDGDDFVCVKCGAKLHSDLEVATANLALVAITGQSIP |
| | | RPPREQSSGKKSTGTARMKKTSGETQGKGSKACVSEALNKIE |
| | | QGTARDPVYNPLNSQVSCPAP |
| CasΦ.24 | SEQ ID | VYNPDMKKPNNIRRIREEHFEGLCFGKDVLTKAGKIYEKDGE |
| | NO: 297 | EAAIDFLMGKDEEDPPNFKPPAKTTIVAQSRPFDQWPIYQVSQ |
| | | AVQERVFAYTEEEFNASKEALFSGDISSKSRDFWFKTNNISDQ |
| | | GIGAQGLNTILSHAFSRYSGVIKKVENRNKKRLKKLSKKNQL |
| | | KIEEGLEILEFKPDSAFNENGLLAQPPGINPNIYGYQAVTPFVF |
| | | DPDNPGDVILPKQYEGYSRKPDDIIEKGPSRLDIPKGQPGYVPE |
| | | HQRKNLKKKGRVRLYRRTPPKTKALASILAVLQIGKDWVLFD |
| | | MRGLLRSVYMREAATPGQISAKDLLDTFTGCPVLNTRTGEFT |
| | | FCYKLRSEGALHARKIYTKGETRTLLTSLTSENNTIALVTVDL |
| | | GQRNPAAIMISRLSRKEELSEKDIQPVSRRLLPDRYLNELKRY |
| | | RDAYDAFRQEVRDEAFTSLCPEHQEQVQQYEALTPEKAKNL |
| | | VLKHFFGTHDPDLPWDDMTSNTHYIANLYLERGGDPSKVFFT |
| | | RPLKKDSKSKKPRKPTKRTDASISRLPEIRPKMPEDARKAFEK |
| | | AKWEIYTGHEKFPKLAKRVNQLCREIANWIEKEAKRLTLCDT |
| | | VVVGIEDLSLPPKRGKGKFQETWQGFFRQKFENRWVIDTLKK |
| | | AIQNRAHDKGKYVLGLAPYWTSQRCPACGFIHKSNRNGDHF |
| | | KCLKCEALFHADSEVATWNLALVAVLGKGITNPDSKKPSGQ |
| | | KKTGTTRKKQIKGKNKGKETVNVPPTTQEVEDIIAFFEKDDET |
| | | VRNPVYKPTGT |
| CasΦ.25 | SEQ ID | MKKPNNIRRIREEHFEGLCFGKDVLTKAGKIYEKDGEEAAIDF |
| | NO: 298 | LMGKDEEDPPNFKPPAKTTIVAQSRPFDQWPIYQVSQAVQER |
| | | VFAYTEEEFNASKEALFSGDISSKSRDFWFKTNNISDQGIGAQ |
| | | GLNTILSHAFSRYSGVIKKVENRNKKRLKKLSKKNQLKIEEGL |
| | | EILEFKPDSAFNENGLLAQPPGINPNIYGYQAVTPFVFDPDNPG |
| | | DVILPKQYEGYSRKPDDIIEKGPSRLDIPKGQPGYVPEHQRKN |
| | | LKKKGRVRLYRRTPPKTKALASILAVLQIGKDWVLFDMRGLL |
| | | RSVYMREAATPGQISAKDLLDTFTGCPVLNTRTGEFTFCYKL |
| | | RSEGALHARKIYTKGETRTLLTSLTSENNTIALVTVDLGQRNP |
| | | AAIMISRLSRKEELSEKDIQPVSRRLLPDRYLNELKRYRDAYD |
| | | AFRQEVRDEAFTSLCPEHQEQVQQYEALTPEKAKNLVLKHFF |
| | | GTHDPDLPWDDMTSNTHYIANLYLERGGDPSKVFFTRPLKKD |
| | | SKSKKPRKPTKRTDASISRLPEIRPKMPEDARKAFEKAKWEIY |
| | | TGHEKFPKLAKRVNQLCREIANWIEKEAKRLTLCDTVVVGIE |
| | | DLSLPPKRGKGKFQETWQGFFRQKFENRWVIDTLKKAIQNRA |
| | | HDKGKYVLGLAPYWTSQRCPACGFIHKSNRNGDHFKCLKCE |
| | | ALFHADSEVATWNLALVAVLGKGITNPDSKKPSGQKKTGTT |
| | | RKKQIKGKNKGKETVNVPPTTQEVEDIIAFFEKDDETVRNPVY |
| | | KPTGT |
| CasΦ.26 | SEQ ID | VIKTHFPAGRFRKDHQKTAGKKLKHEGEEACVEYLRNKVSD |
| | NO: 299 | YPPNFKPPAKGTIVAQSRPFSEWPIVRASEAIQKYVYGLTVAE |
| | | LDVFSPGTSKPSHAEWFAKTGVENYGYRQVQGLNTIFQNTVN |
| | | RFKGVLKKVENRNKKSLKRQEGANRRRVEEGLPEVPVTVES |
| | | ATDDEGRLLQPPGVNPSIYGYQGVAPRVCTDLQGFSGMSVDF |
| | | AGYRRDPDAVLVESLPEGRLSIPKGERGYVPEWQRDPERNKF |
| | | PLREGSRRQRKWYSNACHKPKPGRTSKYDPEALKKASAKDA |
| | | LLVSISIGEDWAIIDVRGLLRDARRRGFTPEEGLSLNSLLGLFT |
| | | EYPVFDVQRGLITFTYKLGQVDVHSRKTVPTFRSRALLESLVA |
| | | KEEIALVSVDLGQTNPASMKVSRVRAQEGALVAEPVHRMFLS |
| | | DVLLGELSSYRKRMDAFEDAIRAQAFETMTPEQQAEITRVCD |
| | | VSVEVARRRVCEKYSISPQDVPWGEMTGHSTFIVDAVLRKGG |
| | | DESLVYFKNKEGETLKFRDLRISRMEGVRPRLTKDTRDALNK |
| | | AVLDLKRAHPTFAKLAKQKLELARRCVNFIEREAKRYTQCER |
| | | VVFVIEDLNVGFFHGKGKRDRGWDAFFTAKKENRWVIQALH |
| | | KAFSDLGLHRGSYVIEVTPQRTSMTCPRCGHCDKGNRNGEKF |
| | | VCLQCGATLHADLEVATDNIERVALTGKAMPKPPVRERSGD |
| | | VQKAGTARKARKPLKPKQKTEPSVQEGSSDDGVDKSPGDAS |
| | | RNPVYNPSDTLSI |
| CasΦ.27 | SEQ ID | MAKAKTLAALLRELLPGQHLAPHHRWVANKLLMTSGDAAA |
| | NO: 300 | FVIGKSVSDPVRGSFRKDVITKAGRIFKKDGPDAAAAFLDGK |
| | | WEDRPPNFQPPAKAAIVAISRSFDEWPIVKVSCAIQQYLYALP |
| | | VQEFESSVPEARAQAHAAWFQDTGVDDCNFKSTQGLNAIFN |
| | | HGKRTYEGVLKKAQNRNDKKNLRLERINAKRAEAGQAPLVA |
| | | GPDESPTDDAGCLLHPPGINANIYCYQQVSPRPYEQSCGIQLPP |
| | | EYAGYNRLSNVAIPPMPNRLDIPQGQPGYVPEHHRHGIKKFG |
| | | RVRKRYGVVPGRNRDADGKRTRQVLTEAGAAAKARDSVLA |
| | | VIRIGDDWTVVDLRGLLRNAQWRKLVPDGGITVQGLLDLFTG |
| | | DPVIDPRRGVVTFIYKADSVGIHSEKVCRGKQSKNLLERLCA |
| | | MPEKSSTRLDCARQAVALVSVDLGQRNPVAARFSRVSLAEG |
| | | QLQAQLVSAQFLDDAMVAMIRSYREEYDRFESLVREQAKAA |
| | | LSPEQLSEIVRHEADSAESVKSCVCAKFGIDPAGLSWDKMTSG |
| | | TWRIADHVQAAGGDVEWFFFKTCGKGKEIKTVRRSDFNVAK |
| | | QFRLRLSPETRKDWNDAIWELKRGNPAYVSFSKRKSEFARRV |
| | | VNDLVHRARRAVRCDEVVFAIEDLNISFFHGKGQRQMGWDA |
| | | FFEVKQENRWFIQALHKAFVERATHKGGYVLEVAPARTSTTC |
| | | PECRHCDPESRRGEQFCCIKCRHTCHADLEVATFNIEQVALTG |
| | | VSLPKRLSSTLL |
| CasΦ.28 | SEQ ID | MSKEKTPPSAYAILKAKHFPDLDFEKKHKMMAGRMFKNGAS |
| | NO: 301 | EQEVVQYLQGKGSESLMDVKPPAKSPILAQSRPFDEWEMVRT |
| | | SRLIQETIFGIPKRGSIPKRDGLSETQFNELVASLEVGGKPMLN |
| | | KQTRAIFYGLLGIKPPTFHAMAQNILIDLAINIRKGVLKKVDNL |
| | | NEKNRKKVKRIRDAGEQDVMVPAEVTAHDDRGYLNHPPGV |
| | | NPTIPGYQGVVIPFPEGFEGLPSGMTPVDWSHVLVDYLPHDRL |
| | | SIPKGSPGYIPEWQRPLLNRHKGRRHRSWYANSLNKPRKSRT |
| | | EEAKDRQNAGKRTALIEAERLKGVLPVLMRFKEDWLIIDARG |
| | | LLRNARYRGVLPEGSTLGNLIDLFSDSPRVDTRRGICTFLYRK |
| | | GRAYSTKPVKRKESKETLLKLTEKSTIALVSIDLGQTNPLTAK |
| | | LSKVRQVDGCLVAEPVLRKLIDNASEDGKEIARYRVAHDLLR |
| | | ARILEDAIDLLGIYKDEVVRARSDTPDLCKERVCRFLGLDSQA |
| | | IDWDRMTPYTDFIAQAFVAKGGDPKVVTIKPNGKPKMFRKD |
| | | RSIKNMKGIRLDISKEASSAYREAQWAIQRESPDFQRLAVWQS |
| | | QLTKRIVNQLVAWAKKCTQCDTVVLAFEDLNIGMMHGSGK |
| | | WANGGWNALFLHKQENRWFMQAFHKALTELSAHKGIPTIEV |
| | | LPHRTSITCTQCGHCHPGNRDGERFKCLKCEFLANTDLEIATD |
| | | NIERVALTGLPMPKGERSSAKRKPGGTRKTKKSKHSGNSPLA |
| | | AE |
| CasΦ.29 | SEQ ID | MEKAGPTSPLSVLIHKNFEGCRFQIDHLKIAGRKLAREGEAAA |
| | NO: 302 | IEYLLDKKCEGLPPNFQPPAKGNVIAQSRPFTEWAPYRASVAI |
| | | QKYIYSLSVDERKVCDPGSSSDSHEKWFKQTGVQNYGYTHV |
| | | QGLNLIFKHALARYDGVLKKVDNRNEKNRKKAERVNSFRRE |
| | | EGLPEEVFEEEKATDETGHLLQPPGVNHSIYCYQSVRPKPFNP |
| | | RKPGGISLPEAYSGYSLKPQDELPIGSLDRLSIPPGQPGYVPEW |
| | | QRSQLTTQKHRRKRSWYSAQKWKPRTGRTSTFDPDRLNCAR |
| | | AQGAILAVVRIHEDWVVFDVRGLLRNALWRELAGKGLTVRD |
| | | LLDFFTGDPVVDTKRGVVTFTYKLGKVDVHSLRTVRGKRSK |
| | | KVLEDLTLSSDVGLVTIDLGQTNVLAADYSKVTRSENGELLA |
| | | VPLSKSFLPKHLLHEVTAYRTSYDQMEEGFRRKALLTLTEDQ |
| | | QVEVTLVRDFSVESSKTKLLQLGVDVTSLPWEKMSSNTTYIS |
| | | DQLLQQGADPASLFFDGERDGKPCRHKKKDRTWAYLVRPKV |
| | | SPETRKALNEALWALKNTSPEFESLSKRKIQFSRRCMNYLLNE |
| | | AKRISGCGQVVFVIEDLNVRVHHGRGKRAIGWDNFFKPKREN |
| | | RWFMQALHKAASELAIHRGMHIIEACPARSSITCPKCGHCDPE |
| | | NRCSSDREKFLCVKCGAAFHADLEVATFNLRKVALTGTALPK |
| | | SIDHSRDGLIPKGARNRKLKEPQANDEKACA |
| CasΦ.30 | SEQ ID | MKEQSPLSSVLKSNFPGKKFLSADIRVAGRKLAQLGEAAAVE |
| | NO: 303 | YLSPRQRDSVPNFRPPAFCTVVAKSRPFEEWPIYKASVLLQEQ |
| | | IYGMTGQEFEERCGSIPTSLSGLRQWASSVGLGAAMEGLHVQ |
| | | GMNLMVKNAINRYKGVLVKVENRNKKLVEANEAKNSSREE |
| | | RGLPPLRPPELGSAFGPDGRLVNPPGIDKSIRLYQGVSPVPVVK |
| | | TTGRPTVHRLDIPAGEKGHVPLWQREAGLVKEGPRRRRMWY |
| | | SNSNLKRSRKDRSAEASEARKADSVVVRVSVKEDWVDIDVR |
| | | GLLRNVAWRGIERAGESTEDLLSLFSGDPVVDPSRDSVVFLY |
| | | KEGVVDVLSKKVVGAGKSRKQLEKMVSEGPVALVSCDLGQT |
| | | NYVAARVSVLDESLSPVRSFRVDPREFPSADGSQGVVGSLDRI |
| | | RADSDRLEAKLLSEAEASLPEPVRAEIEFLRSERPSAVAGRLCL |
| | | KLGIDPRSIPWEKMGSTTSFISEALSAKGSPLALHDGAPIKDSR |
| | | FAHAARGRLSPESRKALNEALWERKSSSREYGVISRRKSEASR |
| | | RMANAVLSESRRLTGLAVVAVNLEDLNMVSKFFHGRGKRAP |
| | | GWAGFFTPKMENRWFIRSIHKAMCDLSKHRGITVIESRPERTS |
| | | ISCPECGHCDPENRSGERFSCKSCGVSLHADFEVATRNLERVA |
| | | LTGKPMPRRENLHSPEGATASRKTRKKPREATASTFLDLRSVL |
| | | SSAENEGSGPAARAG |
| CasΦ.31 | SEQ ID | MLPPSNKIGKSMSLKEFINKRNFKSSIIKQAGKILKKEGEEAVK |
| | NO: 304 | KYLDDNYVEGYKKRDFPITAKCNIVASNRKIEDFDISKFSSFIQ |
| | | NYVFNLNKDNFEEFSKIKYNRKSFDELYKKIANEIGLEKPNYE |
| | | NIQGEIAVIRNAINIYNGVLKKVENRNKKIQEKNQSKDPPKLL |
| | | SAFDDNGFLAERPGINETIYGYQSVRLRHLDVEKDKDIIVQLP |
| | | DIYQKYNKKSTDKISVKKRLNKYNVDEYGKLISKRRKERINK |
| | | DDAILCVSNFGDDWIIFDARGLLRQTYRYKLKKKGLCIKDLL |
| | | NLFTGDPIINPTKTDLKEALSLSFKDGIINNRTLKVKNYKKCPE |
| | | LISELIRDKGKVAMISIDLGQTNPISYRLSKFTANNVAYIENGVI |
| | | SEDDIVKMKKWREKSDKLENLIKEEAIASLSDDEQREVRLYE |
| | | NDIADNTKKKILEKFNIREEDLDFSKMSNNTYFIRDCLKNKNI |
| | | DESEFTFEKNGKKLDPTDACFAREYKNKLSELTRKKINEKIWE |
| | | IKKNSKEYHKISIYKKETIRYIVNKLIKQSKEKSECDDIIVNIEK |
| | | LQIGGNFFGGRGKRDPGWNNFFLPKEENRWFINACHKAFSEL |
| | | APHKGIIVIESDPAYTSQTCPKCENCDKENRNGEKFKCKKCNY |
| | | EANADIDVATENLEKIAKNGRRLIKNFDQLGERLPGAEMPGG |
| | | ARKRKPSKSLPKNGRGAGVGSEPELINQSPSQVIA |
| CasΦ.32 | SEQ ID | VPDKKETPLVALCKKSFPGLRFKKHDSRQAGRILKSKGEGAA |
| | NO: 305 | VAFLEGKGGTTQPNFKPPVKCNIVAMSRPLEEWPIYKASVVIQ |
| | | KYVYAQSYEEFKATDPGKSEAGLRAWLKATRVDTDGYFNV |
| | | QGLNLIFQNARATYEGVLKKVENRNSKKVAKIEQRNEHRAER |
| | | GLPLLTLDEPETALDETGHLRHRPGINCSVFGYQHMKLKPYV |
| | | PGSIPGVTGYSRDPSTPIAACGVDRLEIPEGQPGYVPPWDREN |
| | | LSVKKHRRKRASWARSRGGAIDDNMLLAVVRVADDWALLD |
| | | LRGLLRNTQYRKLLDRSVPVTIESLLNLVTNDPTLSVVKKPGK |
| | | PVRYTATLIYKQGVVPVVKAKVVKGSYVSKMLDDTTETFSL |
| | | VGVDLGVNNLIAANALRIRPGKCVERLQAFTLPEQTVEDFFRF |
| | | RKAYDKHQENLRLAAVRSLTAEQQAEVLALDTFGPEQAKMQ |
| | | VCGHLGLSVDEVPWDKVNSRSSILSDLAKERGVDDTLYMFPF |
| | | FKGKGKKRKTEIRKRWDVNWAQHFRPQLTSETRKALNEAK |
| | | WEAERNSSKYHQLSIRKKELSRHCVNYVIRTAEKRAQCGKVI |
| | | VAVEDLHHSFRRGGKGSRKSGWGGFFAAKQEGRWLMDALF |
| | | GAFCDLAVHRGYRVIKVDPYNTSRTCPECGHCDKANRDRVN |
| | | REAFICVCCGYRGNADIDVAAYNIAMVAITGVSLRKAARASV |
| | | ASTPLESLAAE |
| CasΦ.33 | SEQ ID | MSKTKELNDYQEALARRLPGVRHQKSVRRAARLVYDRQGE |
| | NO: 306 | DAMVAFLDGKEVDEPYTLQPPAKCHILAVSRPIEEWPIARVT |
| | | MAVQEHVYALPVHEVEKSRPETTEGSRSAWFKNSGVSNHGV |
| | | THAQTLNAILKNAYNVYNGVIKKVENRNAKKRDSLAAKNKS |
| | | RERKGLPHFKADPPELATDEQGYLLQPPSPNSSVYLVQQHLR |
| | | TPQIDLPSGYTGPVVDPRSPIPSLIPIDRLAIPPGQPGYVPLHDR |
| | | EKLTSNKHRRMKLPKSLRAQGALPVCFRVFDDWAVVDGRGL |
| | | LRHAQYRRLAPKNVSIAELLELYTGDPVIDIKRNLMTFRFAEA |
| | | VVEVTARKIVEKYHNKYLLKLTEPKGKPVREIGLVSIDLNVQ |
| | | RLIALAIYRVHQTGESQLALSPCLHREILPAKGLGDFDKYKSK |
| | | FNQLTEEILTAAVQTLTSAQQEEYQRYVEESSHEAKADLCLK |
| | | YSITPHELAWDKMTSSTQYISRWLRDHGWNASDFTQITKGRK |
| | | KVERLWSDSRWAQELKPKLSNETRRKLEDAKHDLQRANPEW |
| | | QRLAKRKQEYSRHLANTVLSMAREYTACETVVIAIENLPMKG |
| | | GFVDGNGSRESGWDNFFTHKKENRWMIKDIHKALSDLAPNR |
| | | GVHVLEVNPQYTSQTCPECGHRDKANRDPIQRERFCCTHCGA |
| | | QRHADLEVATHNIAMVATTGKSLTGKSLAPQRLQEAAE |
| CasΦ.41 | SEQ ID | VLLSDRIQYTDPSAPIPAMTVVDRRKIKKGEPGYVPPFMRKNL |
| | NO: 307 | STNKHRRMRLSRGQKEACALPVGLRLPDGKDGWDFIIFDGRA |
| | | LLRACRRLRLEVTSMDDVLDKFTGDPRIQLSPAGETIVTCMLK |
| | | PQHTGVIQQKLITGKMKDRLVQLTAEAPIAMLTVDLGEHNLV |
| | | ACGAYTVGQRRGKLQSERLEAFLLPEKVLADFEGYRRDSDEH |
| | | SETLRHEALKALSKRQQREVLDMLRTGADQARESLCYKYGL |
| | | DLQALPWDKMSSNSTFIAQHLMSLGFGESATHVRYRPKRKAS |
| | | ERTILKYDSRFAAEEKIKLTDETRRAWNEAIWECQRASQEFRC |
| | | LSVRKLQLARAAVNWTLTQAKQRSRCPRVVVVVEDLNVRF |
| | | MHGGGKRQEGWAGFFKARSEKRWFIQALHKAYTELPTNRGI |
| | | HVMEVNPARTSITCTKCGYCDPENRYGEDFHCRNPKCKVRG |
| | | GHVANADLDIATENLARVALSGPMPKAPKLK |
| CasΦ.34 | SEQ ID | MTPSFGYQMIIVTPIHHASGAWATLRLLFLNPKTSGVMLGMT |
| | NO: 308 | KTKSAFALMREEVFPGLLFKSADLKMAGRKFAKEGREAAIEY |
| | | LRGKDEERPANFKPPAKGDIIAQSRPFDQWPIVQVSQAIQKYIF |
| | | GLTKAEFDATKTLLYGEGNHPTTESRRRWFEATGVPDFGFTS |
| | | AQGLNAIFSSALARYEGVIQKVENRNEKRLKKLSEKNQRLVE |
| | | EGHAVEAYVPETAFHTLESLKALSEKSLVPLDDLMDKIDRLA |
| | | QPPGINPCLYGYQQVAPYIYDPENPRGVVLPDLYLGYCRKPD |
| | | DPITACPNRLDIPKGQPGYIPEHQRGQLKKHGRVRRFRYTNPQ |
| | | AKARAKAQTAILAVLRIDEDWVVMDLRGLLRNVYFREVAAP |
| | | GELTARTLLDTFTGCPVLNLRSNVVTFCYDIESKGALHAEYV |
| | | RKGWATRNKLLDLTKDGQSVALLSVDLGQRHPVAVMISRLK |
| | | RDDKGDLSEKSIQVVSRTFADQYVDKLKRYRVQYDALRKEIY |
| | | DAALVSLPPEQQAEIRAYEAFAPGDAKANVLSVMFQGEVSPD |
| | | ELPWDKMNTNTHYISDLYLRRGGDPSRVFFVPQPSTPKKNAK |
| | | KPPAPRKPVKRTDENVSHMPEFRPHLSNETREAFQKAKWTM |
| | | ERGNVRYAQLSRFLNQIVREANNWLVSEAKKLTQCQTVVWA |
| | | IEDLHVPFFHGKGKYHETWDGFFRQKKEDRWFVNVFHKAISE |
| | | RAPNKGEYVMEVAPYRTSQRCPVCGFVDADNRHGDHFKCLR |
| | | CGVELHADLEVATWNIALVAVQGHGIAGPPREQSCGGETAG |
| | | TARKGKNIKKNKGLADAVTVEAQDSEGGSKKDAGTARNPVY |
| | | IPSESQVNCPAP |
| CasΦ.35 | SEQ ID | MKPKTPKPPKTPVAALIDKHFPGKRFRASYLKSVGKKLKNQG |
| | NO: 309 | EDVAVRFLTGKDEERPPNFQPPAKSNIVAQSRPIEEWPIHKVS |
| | | VAVQEYVYGLTVAEKEACSDAGESSSSHAAWFAKTGVENFG |
| | | YTSVQGLNKIFPPTFNRFDGVIKKVENRNEKKRQKATRINEAK |
| | | RNKGQSEDPPEAEVKATDDAGYLLQPPGINHSVYGYQSITLCP |
| | | YTAEKFPTIKLPEEYAGYHSNPDAPIPAGVPDRLAIPEGQPGH |
| | | VPEEHRAGLSTKKHRRVRQWYAMANWKPKPKRTSKPDYDR |
| | | LAKARAQGALLIVIRIDEDWVVVDARGLLRNVRWRSLGKREI |
| | | TPNELLDLFTGDPVLDLKRGVVTFTYAEGVVNVCSRSTTKGK |
| | | QTKVLLDAMTAPRDGKKRQIGMVAVDLGQTNPIAAEYSRVG |
| | | KNAAGTLEATPLSRSTLPDELLREIALYRKAHDRLEAQLREEA |
| | | VLKLTAEQQAENARYVETSEEGAKLALANLGVDTSTLPWDA |
| | | MTGWSTCISDHLINHGGDTSAVFFQTIRKGTKKLETIKRKDSS |
| | | WADIVRPRLTKETREALNDFLWELKRSHEGYEKLSKRLEELA |
| | | RRAVNHVVQEVKWLTQCQDIVIVIEDLNVRNFHGGGKRGGG |
| | | WSNFFTVKKENRWFMQALHKAFSDLAAHRGIPVLEVYPART |
| | | SITCLGCGHCDPENRDGEAFVCQQCGATFHADLEVATRNIAR |
| | | VALTGEAMPKAPAREQPGGAKKRGTSRRRKLTEVAVKSAEP |
| | | TIHQAKNQQLNGTSRDPVYKGSELPAL |
| CasΦ.43 | SEQ ID | MSEITDLLKANFKGKTFKSADMRMAGRILKKSGAQAVIKYLS |
| | NO: 310 | DKGAVDPPDFRPPAKCNIIAQSRPFDEWPICKASMAIQQHIYG |
| | | LTKNEFDESSPGTSSASHEQWFAKTGVDTHGFTHVQGLNLIF |
| | | QHAKKRYEGVIKKVENYNEKERKKFEGINERRSKEGMPLLEP |
| | | RLRTAFGDDGKFAEKPGVNPSIYLYQQTSPRPYDKTKHPYVH |
| | | APFELKEITTIPTQDDRLKIPFGAPGHVPEKHRSQLSMAKHKR |
| | | RRAWYALSQNKPRPPKDGSKGRRSVRDLADLKAASLADAIPL |
| | | VSRVGFDWVVIDGRGLLRNLRWRKLAHEGMTVEEMLGFFSG |
| | | DPVIDPRRNVATFIYKAEHATVKSRKPIGGAKRAREELLKATA |
| | | SSDGVIRQVGLISVDLGQTNPVAYEISRMHQANGELVAEHLE |
| | | YGLLNDEQVNSIQRYRAAWDSMNESFRQKAIESLSMEAQDEI |
| | | MQASTGAAKRTREAVLTMFGPNATLPWSRMSSNTTCISDALI |
| | | EVGKEEETNFVTSNGPRKRTDAQWAAYLRPRVNPETRALLN |
| | | QAVWDLMKRSDEYERLSKRKLEMARQCVNFVVARAEKLTQ |
| | | CNNIGIVLENLVVRNFHGSGRRESGWEGFFEPKRENRWFMQV |
| | | LHKAFSDLAQHRGVMVFEVHPAYSSQTCPACRYVDPKNRSS |
| | | EDRERFKCLKCGRSFNADREVATFNIREIARTGVGLPKPDCER |
| | | SRGVQTTGTARNPGRSLKSNKNPSEPKRVLQSKTRKKITSTET |
| | | QNEPLATDLKT |
| CasΦ.44 | SEQ ID | MTPKTESPLSALCKKHFPGKRFRTNYLKDAGKILKKHGEDAV |
| | NO: 311 | VAFLSDKQEDEPANFCPPAKVHILAQSRPFEDWPINLASKAIQ |
| | | TYVYGLTADERKTCEPGTSKESHDRWFKETGVDHHGFTSVQ |
| | | GLNLIFKHTLNRYDGVIKKVETRNEKRRSSVVRINEKKAAEG |
| | | LPLIAAEAEETAFGEDGRLLQPPGVNHSIYCFQQVSPQPYSSK |
| | | KHPQVVLPHAVQGVDPDAPIPVGRPNRLDIPKGQPGYVPEWQ |
| | | RPHLSMKCKRVRMWYARANWRRKPGRRSVLNEARLKEASA |
| | | KGALPIVLVIGDDWLVMDARGLLRSVFWRRVAKPGLSLSELL |
| | | NVTPTGLFSGDPVIDPKRGLVTFTSKLGVVAVHSRKPTRGKKS |
| | | KDLLLKMTKPTDDGMPRHVGMVAIDLGQTNPVAAEYSRVV |
| | | QSDAGTLKQEPVSRGVLPDDLLKDVARYRRAYDLTEESIRQE |
| | | AIALLSEGHRAEVTKLDQTTANETKRLLVDRGVSESLPWEKM |
| | | SSNTTYISDCLVALGKTDDVFFVPKAKKGKKETGIAVKRKDH |
| | | GWSKLLRPRTSPEARKALNENQWAVKRASPEYERLSRRKLEL |
| | | GRRCVNHIIQETKRWTQCEDIVVVLEDLNVGFFHGSGKRPDG |
| | | WDNFFVSKRENRWFIQVLHKAFGDLATHRGTHVIEVHPARTS |
| | | ITCIKCGHCDAGNRDGESFVCLASACGDRRHADLEVATRNVA |
| | | RVAITGERMPPSEQARDVQKAGGARKRKPSARNVKSSYPAV |
| | | EPAPASP |
| CasΦ.36 | SEQ ID | MSDNKMKKLSKEEKPLTPLQILIRKYIDKSQYPSGFKTTIIKQA |
| | NO: 312 | GVRIKSVKSEQDEINLANWIISKYDPTYIKRDFNPSAKCQIIATS |
| | | RSVADFDIVKMSNKVQEIFFASSHLDKNVFDIGKSKSDHDSW |
| | | FERNNVDRGIYTYSNVQGMNLIFSNTKNTYLGVAVKAQNKFS |
| | | SKMKRIQDINNFRITNHQSPLPIPDEIKIYDDAGFLLNPPGVNP |
| | | NIFGYQSCLLKPLENKEIISKTSFPEYSRLPADMIEVNYKISNRL |
| | | KFSNDQKGFIQFKDKLNLFKINSQELFSKRRRLSGQPILLVASF |
| | | GDDWVVLDGRGLLRQVYYRGIAKPGSITISELLGFFTGDPIVD |
| | | PIRGVVSLGFKPGVLSQETLKTTSARIFAEKLPNLVLNNNVGL |
| | | MSIDLGQTNPVSYRLSEITSNMSVEHICSDFLSQDQISSIEKAKT |
| | | SLDNLEEEIAIKAVDHLSDEDKINFANFSKLNLPEDTRQSLFEK |
| | | YPELIGSKLDFGSMGSGTSYIADELIKFENKDAFYPSGKKKFD |
| | | LSFSRDLRKKLSDETRKSYNDALFLEKRTNDKYLKNAKRRKQ |
| | | IVRTVANSLVSKIEELGLTPVINIENLAMSGGFFDGRGKREKG |
| | | WDNFFKVKKENRWVMKDFHKAFSELSPHHGVIVIESPPYCTS |
| | | VTCTKCNFCDKKNRNGHKFTCQRCGLDANADLDIATENLEK |
| | | VAISGKRMPGSERSSDERKVAVARKAKSPKGKAIKGVKCTIT |
| | | DEPALLSANSQDCSQSTS |
| CasΦ.37 | SEQ ID | MALSLAEVRERHFKGLRFRSSYLKRAGKILKKEGEAACVAYL |
| | NO: 313 | TGKDEESPPNFKPPAKCDVVAQSRPFEEWPIVQASVAVQSYV |
| | | YGLTKEAFEAFNPGTTKQSHEACLAATGIDTCGYSNVQGLNL |
| | | IFRQAKNRYEGVITKVENRNKKAKKKLTRKNEWRQKNGHSE |
| | | LPEAPEELTFNDEGRLLQPPGINPSLYTYQQISPTPWSPKDSSIL |
| | | PPQYAGYERDPNAPIPFGVAKDRLTIASGCPGYIPEWMRTAGE |
| | | KTNPRTQKKFMHPGLSTRKNKRMRLPRSVRSAPLGALLVTIH |
| | | LGEDWLVLDVRGLLRNARWRGVAPKDISTQGLLNLFTGDPVI |
| | | DTRRGVVTFTYKPETVGIHSRTWLYKGKQTKEVLEKLTQDQT |
| | | VALVAIDLGQTNPVSAAASRVSRSGENLSIETVDRFFLPDELIK |
| | | ELRLYRMAHDRLEERIREESTLALTEAQQAEVRALEHVVRDD |
| | | AKNKVCAAFNLDAASLPWDQMTSNTTYLSEAILAQGVSRDQ |
| | | VFFTPNPKKGSKEPVEVMRKDRAWVYAFKAKLSEETRKAKN |
| | | EALWALKRASPDYARLSKRREELCRRSVNMVINRAKKRTQC |
| | | QVVIPVLEDLNIGFFHGSGKRLPGWDNFFVAKKENRWLMNG |
| | | LHKSFSDLAVHRGFYVFEVMPHRTSITCPACGHCDSENRDGE |
| | | AFVCLSCKRTYHADLDVATHNLTQVAGTGLPMPEREHPGGT |
| | | KKPGGSRKPESPQTHAPILHRTDYSESADRLGS |
| CasΦ.45 | SEQ ID | QAVIKYLSDKGAVDPPDFRPPAKCNIIAQSRPFDEWPICKASM |
| | NO: 314 | AIQQHIYGLTKNEFDESSPGTSSASHEQWFAKTGVDTHGFTH |
| | | VQGLNLIFQHAKKRYEGVIKKVENYNEKERKKFEGINERRSK |
| | | EGMPLLEPRLRTAFGDDGKFAEKPGVNPSIYLYQQTSPRPYD |
| | | KTKHPYVHAPFELKEITTIPTQDDRLKIPFGAPGHVPEKHRSQL |
| | | SMAKHKRRRAWYALSQNKPRPPKDGSKGRRSVRDLADLKA |
| | | ASLADAIPLVSRVGFDWVVIDGRGLLRNLRWRKLAHEGMTV |
| | | EEMLGFFSGDPVIDPRRNVATFIYKAEHATVKSRKPIGGAKRA |
| | | REELLKATASSDGVIRQVGLISVDLGQTNPVAYEISRMHQAN |
| | | GELVAEHLEYGLLNDEQVNSIQRYRAAWDSMNESFRQKAIES |
| | | LSMEAQDEIMQASTGAAKRTREAVLTMFGPNATLPWSRMSS |
| | | NTTCISDALIEVGKEEETNFVTSNGPRKRTDAQWAAYLRPRV |
| | | NPETRALLNQAVWDLMKRSDEYERLSKRKLEMARQCVNFV |
| | | VARAEKLTQCNNIGIVLENLVVRNFHGSGRRESGWEGFFEPK |
| | | RENRWFMQVLHKAFSDLAQHRGVMVFEVHPAYSSQTCPACR |
| | | YVDPKNRSSEDRERFKCLKCGRSFNADREVATFNIREIARTGV |
| | | GLPKPDCERSRDVQTPGTARKSGRSLKSQDNLSEPKRVLQSK |
| | | TRKKITSTETQNEPLATDLKT |
| CasΦ.38 | SEQ ID | MIKEQSELSKLIEKYYPGKKFYSNDLKQAGKHLKKSEHLTAK |
| | NO: 315 | ESEELTVEFLKSCKEKLYDFRPPAKALIISTSRPFEEWPIYKASE |
| | | SIQKYIYSLTKEELEKYNISTDKTSQENFFKESLIDNYGFANVS |
| | | GLNLIFQHTKAIYDGVLKKVNNRNNKILKKYKRKIEEGIEIDSP |
| | | ELEKAIDESGHFINPPGINKNIYCYQQVSPTIFNSFKETKIICPFN |
| | | YKRNPNDIIQKGVIDRLAIPFGEPGYIPDHQRDKVNKHKKRIR |
| | | KYYKNNENKNKDAILAKINIGEDWVLFDLRGLLRNAYWRKL |
| | | IPKQGITPQQLLDMFSGDPVIDPIKNNITFIYKESIIPIHSESIIKTK |
| | | KSKELLEKLTKDEQIALVSIDLGQTNPVAARFSRLSSDLKPEH |
| | | VSSSFLPDELKNEICRYREKSDLLEIEIKNKAIKMLSQEQQDEI |
| | | KLVNDISSEELKNSVCKKYNIDNSKIPWDKMNGFTTFIADEFI |
| | | NNGGDKSLVYFTAKDKKSKKEKLVKLSDKKIANSFKPKISKE |
| | | TREILNKITWDEKISSNEYKKLSKRKLEFARRATNYLINQAKK |
| | | ATRLNNVVLVVEDLNSKFFHGSGKREDGWDNFFIPKKENRW |
| | | FIQALHKSLTDVSIHRGINVIEVRPERTSITCPKCGCCDKENRK |
| | | GEDFKCIKCDSVYHADLEVATFNIEKVAITGESMPKPDCERLG |
| | | GEESIG |
| CasΦ.39 | SEQ ID | VAFLDGKEVDEPYTLQPPAKCHILAVSRPIEEWPIARVTMAVQ |
| | NO: 316 | EHVYALPVHEVEKSRPETTEGSRSAWFKNSGVSNHGVTHAQ |
| | | TLNAILKNAYNVYNGVIKKVENRNAKKRDSLAAKNKSRERK |
| | | GLPHFKADPPELATDEQGYLLQPPSPNSSVYLVQQHLRTPQID |
| | | LPSGYTGPVVDPRSPIPSLIPIDRLAIPPGQPGYVPLHDREKLTS |
| | | NKHRRMKLPKSLRAQGALPVCFRVFDDWAVVDGRGLLRHA |
| | | QYRRLAPKNVSIAELLELYTGDPVIDIKRNLMTFRFAEAVVEV |
| | | TARKIVEKYHNKYLLKLTEPKGKPVREIGLVSIDLNVQRLIAL |
| | | AIYRVHQTGESQLALSPCLHREILPAKGLGDFDKYKSKFNQLT |
| | | EEILTAAVQTLTSAQQEEYQRYVEESSHEAKADLCLKYSITPH |
| | | ELAWDKMTSSTQYISRWLRDHGWNASDFTQITKGRKKVERL |
| | | WSDSRWAQELKPKLSNETRRKLEDAKHDLQRANPEWQRLA |
| | | KRKQEYSRHLANTVLSMAREYTACETVVIAIENLPMKGGFVD |
| | | GNGSRESGWDNFFTHKKENRWMIKDIHKALSDLAPNRGVHV |
| | | LEVNPQYTSQTCPECGHRDKANRDPIQRERFCCTHCGAQRHA |
| | | DLEVATHNIAMVATTGKSLTGKSLAPQRLQ |
| CasΦ.42 | SEQ ID | LEIPEGEPGHVPWFQRMDIPEGQIGHVNKIQRFNFVHGKNSGK |
| | NO: 317 | VKFSDKTGRVKRYHHSKYKDATKPYKFLEESKKVSALDSILA |
| | | IITIGDDWVVFDIRGLYRNVFYRELAQKGLTAVQLLDLFTGDP |
| | | VIDPKKGIITFSYKEGVVPVFSQKIVSRFKSRDTLEKLTSQGPV |
| | | ALLSVDLGQNEPVAARVCSLKNINDKIALDNSCRIPFLDDYKK |
| | | QIKDYRDSLDELEIKIRLEAINSLDVNQQVEIRDLDVFSADRAK |
| | | ASTVDMFDIDPNLISWDSMSDARFSTQISDLYLKNGGDESRV |
| | | YFEINNKRIKRSDYNISQLVRPKLSDSTRKNLNDSIWKLKRTSE |
| | | EYLKLSKRKLELSRAVVNYTIRQSKLLSGINDIVIILEDLDVKK |
| | | KFNGRGIRDIGWDNFFSSRKENRWFIPAFHKSFSELSSNRGLC |
| | | VIEVNPAWTSATCPDCGFCSKENRDGINFTCRKCGVSYHADI |
| | | DVATLNIARVAVLGKPMSGPADRERLGGTKKPRVARSRKDM |
| | | KRKDISNGTVEVMVTA |
| CasΦ.46 | SEQ ID | IPSFGYLDRLKIAKGQPGYIPEWQRETINPSKKVRRYWATNHE |
| | NO: 318 | KIRNAIPLVVFIGDDWVIIDGRGLLRDARRRKLADKNTTIEQL |
| | | LEMVSNDPVIDSTRGIATLSYVEGVVPVRSFIPIGEKKGREYLE |
| | | KSTQKESVTLLSVDIGQINPVSCGVYKVSNGCSKIDFLDKFFL |
| | | DKKHLDAIQKYRTLQDSLEASIVNEALDEIDPSFKKEYQNINS |
| | | QTSNDVKKSLCTEYNIDPEAISWQDITAHSTLISDYLIDNNITN |
| | | DVYRTVNKAKYKTNDFGWYKKFSAKLSKEAREALNEKIWEL |
| | | KIASSKYKKLSVRKKEIARTIANDCVKRAETYGDNVVVAMES |
| | | LTKNNKVMSGRGKRDPGWHNLGQAKVENRWFIQAISSAFED |
| | | KATHHGTPVLKVNPAYTSQTCPSCGHCSKDNRSSKDRTIFVC |
| | | KSCGEKFNADLDVATYNIAHVAFSGKKLSPPSEKSSATKKPRS |
| | | ARKSKKSRKS |
| CasΦ.47 | SEQ ID | SPIEKLLNGLLVKITFGNDWIICDARGLLDNVQKGIIHKSYFTN |
| | NO: 319 | KSSLVDLIDLFTCNPIVNYKNNVVTFCYKEGVVDVKSFTPIKS |
| | | GPKTQENLIKKLKYSRFQNEKDACVLGVGVDVGVTNPFAING |
| | | FKMPVDESSEWVMLNEPLFTIETSQAFREEIMAYQQRTDEMN |
| | | DQFNQQSIDLLPPEYKVEFDNLPEDINEVAKYNLLHTLNIPNN |
| | | FLWDKMSNTTQFISDYLIQIGRGTETEKTITTKKGKEKILTIRD |
| | | VNWFNTFKPKISEETGKARTEIKRDLQKNSDQFQKLAKSREQ |
| | | SCRTWVNNVTEEAKIKSGCPLIIFVIEALVKDNRVFSGKGHRA |
| | | IGWHNFGKQKNERRWWVQAIHKAFQEQGVNHGYPVILCPPQ |
| | | YTSQTCPKCNHVDRDNRSGEKFKCLKYGWIGNADLDVGAYN |
| | | IARVAITGKALSKPLEQKKIKKAKNKT |
| CasΦ.48 | SEQ ID | LLDNVQKGIIHKSYFTNKSSLVDLIDLFTCNPIVNYKNNVVTF |
| | NO: 320 | CYKEGVVDVKSFTPIKSGPKTQENLIKKLKYSRFQNEKDACV |
| | | LGVGVDVGVTNPFAINGFKMPVDESSEWVMLNEPLFTIETSQ |
| | | AFREEIMAYQQRTDEMNDQFNQQSIDLLPPEYKVEFDNLPEDI |
| | | NEVAKYNLLHTLNIPNNFLWDKMSNTTQFISDYLIQIGRGTET |
| | | EKTITTKKGKEKILTIRDVNWFNTFKPKISEETGKARTEIKRDL |
| | | QKNSDQFQKLAKSREQSCRTWVNNVTEEAKIKSGCPLIIFVIE |
| | | ALVKDNRVFSGKGHRAIGWHNFGKQKNERRWWVQAIHKAF |
| | | QEQGVNHGYPVILCPPQYTSQTCPKCNHVDRDNRSGEKFKCL |
| | | KYGWIGNADLDVGAYNIARVAITGKALSKPLEQKKIKKAKN |
| | | KT |
| CasΦ.49 | SEQ ID | MIKPTVSQFLTPGFKLIRNHSRTAGLKLKNEGEEACKKFVREN |
| | NO: 321 | EIPKDECPNFQGGPAIANIIAKSREFTEWEIYQSSLAIQEVIFTLP |
| | | KDKLPEPILKEEWRAQWLSEHGLDTVPYKEAAGLNLIIKNAV |
| | | NTYKGVQVKVDNKNKNNLAKINRKNEIAKLNGEQEISFEEIK |
| | | AFDDKGYLLQKPSPNKSIYCYQSVSPKPFITSKYHNVNLPEEYI |
| | | GYYRKSNEPIVSPYQFDRLRIPIGEPGYVPKWQYTFLSKKENK |
| | | RRKLSKRIKNVSPILGIICIKKDWCVFDMRGLLRTNHWKKYH |
| | | KPTDSINDLFDYFTGDPVIDTKANVVRFRYKMENGIVNYKPV |
| | | REKKGKELLENICDQNGSCKLATVDVGQNNPVAIGLFELKKV |
| | | NGELTKTLISRHPTPIDFCNKITAYRERYDKLESSIKLDAIKQLT |
| | | SEQKIEVDNYNNNFTPQNTKQIVCSKLNINPNDLPWDKMISGT |
| | | HFISEKAQVSNKSEIYFTSTDKGKTKDVMKSDYKWFQDYKPK |
| | | LSKEVRDALSDIEWRLRRESLEFNKLSKSREQDARQLANWISS |
| | | MCDVIGIENLVKKNNFFGGSGKREPGWDNFYKPKKENRWWI |
| | | NAIHKALTELSQNKGKRVILLPAMRTSITCPKCKYCDSKNRN |
| | | GEKFNCLKCGIELNADIDVATENLATVAITAQSMPKPTCERSG |
| | | DAKKPVRARKAKAPEFHDKLAPSYTVVLREAVKRPAATKK |
| | | AGQAKKKKEF |
| | | (Bold sequence is Nuclear Localization Signal) |

In some embodiments, any of the programmable CasΦ nuclease of the present disclosure (e.g., any one of SEQ ID NO: 274-SEQ ID NO: 321 or fragments or variants thereof) may include a nuclear localization signal (NLS). In some cases, said NLS may have a sequence of KRPAATKKAGQAKKKKEF (SEQ ID NO: 322).

A CasΦ polypeptide or a variant thereof can comprise at least 70%, at least 75%, at least 80%, at least 85%, at least 90%, at least 92%, at least 95%, at least 97%, at least 99%, or 100% sequence identity with any one of SEQ ID NO: 274-SEQ ID NO: 321.

In some embodiments, the Type VI CRISPR/Cas enzyme is a programmable Cas13 nuclease. The general architecture of a Cas13 protein includes an N-terminal domain and two HEPN (higher eukaryotes and prokaryotes nucleotide-binding) domains separated by two helical domains (Liu et al., Cell 2017 Jan. 12; 168(1-2):121-134.e12). The HEPN domains each comprise aR-X4-H motif. Shared features across Cas13 proteins include that upon binding of the crRNA of the guide nucleic acid to a target nucleic acid, the protein undergoes a conformational change to bring together the HEPN domains and form a catalytically active RNase. (Tambe et al., Cell Rep. 2018 Jul. 24; 24(4): 1025-1036.). Thus, two activatable HEPN domains are characteristic of a programmable Cas13 nuclease of the present disclosure. However, programmable Cas13 nucleases also consistent with the present disclosure include Cas13 nucleases comprising mutations in the HEPN domain that enhance the Cas13 proteins cleavage efficiency or mutations that catalytically inactivate the HEPN domains. Programmable Cas13 nucleases consistent with the present disclosure also Cas13 nucleases comprising catalytic

A programmable Cas13 nuclease can be a Cas13a protein (also referred to as “c2c2”), a Cas13b protein, a Cas13c protein, a Cas13d protein, or a Cas13e protein. Example C2c2 proteins are set forth as SEQ ID NO: 130-SEQ ID NO: 137. In some cases, a subject C2c2 protein includes an amino acid sequence having 80% or more (e.g., 85% or more, 90% or more, 95% or more, 98% or more, 99% or more, 99.5% or more, or 100%) amino acid sequence identity with the amino acid sequence set forth in any one of SEQ ID NOs: 130-SEQ ID NO: 137. In some cases, a suitable C2c2 polypeptide comprises an amino acid sequence having at least 80%, at least 85%, at least 90%, at least 95%, at least 98%, at least 99%, or 100%, amino acid sequence identity to the Listeria seeligeri C2c2 amino acid sequence set forth in SEQ ID NO: 130. In some cases, a suitable C2c2 polypeptide comprises an amino acid sequence having at least 80%, at least 85%, at least 90%, at least 95%, at least 98%, at least 99%, or 100%, amino acid sequence identity to the Leptotrichia buccalis C2c2 amino acid sequence set forth in SEQ ID NO: 131. In some cases, a suitable C2c2 polypeptide comprises an amino acid sequence having at least 80%, at least 85%, at least 90%, at least 95%, at least 98%, at least 99%, or 100%, amino acid sequence identity to the Rhodobacter capsulatus C2c2 amino acid sequence set forth in SEQ ID NO: 133. In some cases, a suitable C2c2 polypeptide comprises an amino acid sequence having at least 80%, at least 85%, at least 90%, at least 95%, at least 98%, at least 99%, or 100%, amino acid sequence identity to the Carnobacterium gallinarum C2c2 amino acid sequence set forth in SEQ ID NO: 134. In some cases, a suitable C2c2 polypeptide comprises an amino acid sequence having at least 80%, at least 85%, at least 90%, at least 95%, at least 98%, at least 99%, or 100%, amino acid sequence identity to the Herbinix hemicellulosilytica C2c2 amino acid sequence set forth in SEQ ID NO: 135. In some cases, the C2c2 protein includes an amino acid sequence having 80% or more amino acid sequence identity with the Leptotrichia buccalis (Lbu) C2c2 amino acid sequence set forth in SEQ ID NO: 131. In some cases, the C2c2 protein is a Leptotrichia buccalis (Lbu) C2c2 protein (e.g., see SEQ ID NO: 131). In some cases, the C2c2 protein includes the amino acid sequence set forth in any one of SEQ ID NOs: 130-131 and SEQ ID NOs: 133-137. In some cases, a C2c2 protein used in a method of the present disclosure is not a Leptotrichia shahii (Lsh) C2c2 protein. In some cases, a C2c2 protein used in a method of the present disclosure is not a C2c2 polypeptide having at least 80%, at least 85%, at least 90%, at least 95%, at least 98%, at least 99%, or 100%, amino acid sequence identity to the Lsh C2c2 polypeptide set forth in SEQ ID NO: 132. Other Cas13 protein sequences are set forth in SEQ ID NO: 130-SEQ ID NO: 147.

TABLE 4
Cas13 Protein Sequences

| SEQ | | |
|---|---|---|
| ID | | |
| NO | Description | Sequence |
| SEQ | Listeria | MWISIKTLIHHLGVLFFCDYMYNRREKKIIEVKTMRITKVEVDRKKV |
| ID | seeligeri C2c2 | LISRDKNGGKLVYENEMQDNTEQIMHHKKSSFYKSVVNKTICRPEQ |
| NO: | amino acid | KQMKKLVHGLLQENSQEKIKVSDVTKLNISNFLNHRFKKSLYYFPE |
| 130 | sequence | NSPDKSEEYRIEINLSQLLEDSLKKQQGTFICWESFSKDMELYINWA |
| | | ENYISSKTKLIKKSIRNNRIQSTESRSGQLMDRYMKDILNKNKPFDIQ |
| | | SVSEKYQLEKLTSALKATFKEAKKNDKEINYKLKSTLQNHERQIIEE |
| | | LKENSELNQFNIEIRKHLETYFPIKKTNRKVGDIRNLEIGEIQKIVNHR |
| | | LKNKIVQRILQEGKLASYEIESTVNSNSLQKIKIEEAFALKFINACLFA |
| | | SNNLRNMVYPVCKKDILMIGEFKNSFKEIKHKKFIRQWSQFFSQEIT |
| | | VDDIELASWGLRGAIAPIRNEIIHLKKHSWKKFFNNPTFKVKKSKIIN |
| | | GKTKDVTSEFLYKETLFKDYFYSELDSVPELIINKMESSKILDYYSSD |
| | | QLNQVFTIPNFELSLLTSAVPFAPSFKRVYLKGFDYQNQDEAQPDYN |
| | | LKLNIYNEKAFNSEAFQAQYSLFKMVYYQVFLPQFTTNNDLFKSSV |
| | | DFILTLNKERKGYAKAFQDIRKMNKDEKPSEYMSYIQSQLMLYQKK |
| | | QEEKEKINHFEKFINQVFIKGFNSFIEKNRLTYICHPTKNTVPENDNIE |
| | | IPFHTDMDDSNIAFWLMCKLLDAKQLSELRNEMIKFSCSLQSTEEIST |
| | | FTKAREVIGLALLNGEKGCNDWKELFDDKEAWKKNMSLYVSEELL |
| | | QSLPYTQEDGQTPVINRSIDLVKKYGTETILEKLFSSSDDYKVSAKDI |
| | | AKLHEYDVTEKIAQQESLHKQWIEKPGLARDSAWTKKYQNVINDIS |
| | | NYQWAKTKVELTQVRHLHQLTIDLLSRLAGYMSIADRDFQFSSNYI |
| | | LERENSEYRVTSWILLSENKNKNKYNDYELYNLKNASIKVSSKNDP |
| | | QLKVDLKQLRLTLEYLELFDNRLKEKRNNISHFNYLNGQLGNSILEL |
| | | FDDARDVLSYDRKLKNAVSKSLKEILSSHGMEVTFKPLYQTNHHLK |
| | | IDKLQPKKIHHLGEKSTVSSNQVSNEYCQLVRTLLTMK |
| SEQ | Leptotrichia | MKVTKVGGISHKKYTSEGRLVKSESEENRTDERLSALLNMRLDMYI |
| ID | buccalis (Lbu) | KNPSSTETKENQKRIGKLKKFFSNKMVYLKDNTLSLKNGKKENIDR |
| NO: | C2c2 amino | EYSETDILESDVRDKKNFAVLKKIYLNENVNSEELEVFRNDIKKKLN |
| 131 | acid sequence | KINSLKYSFEKNKANYQKINENNIEKVEGKSKRNIIYDYYRESAKRD |
| | | AYVSNVKEAFDKLYKEEDIAKLVLEIENLTKLEKYKIREFYHEIIGRK |
| | | NDKENFAKIIYEEIQNVNNMKELIEKVPDMSELKKSQVFYKYYLDK |
| | | EELNDKNIKYAFCHFVEIEMSQLLKNYVYKRLSNISNDKIKRIFEYQ |
| | | NLKKLIENKLLNKLDTYVRNCGKYNYYLQDGEIATSDFIARNRQNE |
| | | AFLRNIIGVSSVAYFSLRNILETENENDITGRMRGKTVKNNKGEEKY |
| | | VSGEVDKIYNENKKNEVKENLKMFYSYDFNMDNKNEIEDFFANIDE |
| | | AISSIRHGIVHFNLELEGKDIFAFKNIAPSEISKKMFQNEINEKKLKLK |
| | | IFRQLNSANVFRYLEKYKILNYLKRTRFEFVNKNIPFVPSFTKLYSRI |
| | | DDLKNSLGIYWKTPKTNDDNKTKEIIDAQIYLLKNIYYGEFLNYFMS |
| | | NNGNFFEISKEIIELNKNDKRNLKTGFYKLQKFEDIQEKIPKEYLANI |
| | | QSLYMINAGNQDEEEKDTYIDFIQKIFLKGFMTYLANNGRLSLIYIGS |
| | | DEETNTSLAEKKQEFDKFLKKYEQNNNIKIPYEINEFLREIKLGNILK |
| | | YTERLNMFYLILKLLNHKELTNLKGSLEKYQSANKEEAFSDQLELIN |
| | | LLNLDNNRVTEDFELEADEIGKFLDFNGNKVKDNKELKKFDTNKIY |
| | | FDGENIIKHRAFYNIKKYGMLNLLEKIADKAGYKISIEELKKYSNKK |
| | | NEIEKNHKMQENLHRKYARPRKDEKFTDEDYESYKQAIENIEEYTH |
| | | LKNKVEFNELNLLQGLLLRILHRLVGYTSIWERDLRFRLKGEFPENQ |
| | | YIEEIFNFENKKNVKYKGGQIVEKYIKFYKELHQNDEVKINKYSSAN |
| | | IKVLKQEKKDLYIRNYIAHFNYIPHAEISLLEVLENLRKLLSYDRKLK |
| | | NAVMKSVVDILKEYGFVATFKIGADKKIGIQTLESEKIVHLKNLKKK |
| | | KLMTDRNSEELCKLVKIMFEYKMEEKKSEN |
| SEQ | Leptotrichia | MGNLFGHKRWYEVRDKKDFKIKRKVKVKRNYDGNKYILNINENN |
| ID | shahii (Lsh) | NKEKIDNNKFIRKYINYKKNDNILKEFTRKFHAGNILFKLKGKEGIIR |
| NO: | C2c2 protein | IENNDDFLETEEVVLYIEAYGKSEKLKALGITKKKIIDEAIRQGITKD |
| 132 | | DKKIEIKRQENEEEIEIDIRDEYTNKTLNDCSIILRIIENDELETKKSIYE |
| | | IFKNINMSLYKIIEKIIENETEKVFENRYYEEHLREKLLKDDKIDVILT |
| | | NFMEIREKIKSNLEILGFVKFYLNVGGDKKKSKNKKMLVEKILNINV |
| | | DLTVEDIADFVIKELEFWNITKRIEKVKKVNNEFLEKRRNRTYIKSY |
| | | VLLDKHEKFKIERENKKDKIVKFFVENIKNNSIKEKIEKILAEFKIDEL |
| | | IKKLEKELKKGNCDTEIFGIFKKHYKVNFDSKKFSKKSDEEKELYKII |
| | | YRYLKGRIEKILVNEQKVRLKKMEKIEIEKILNESILSEKILKRVKQY |
| | | TLEHIMYLGKLRHNDIDMTTVNTDDFSRLHAKEELDLELITFFASTN |
| | | MELNKIFSRENINNDENIDFFGGDREKNYVLDKKILNSKIKIIRDLDFI |
| | | DNKNNITNNFIRKFTKIGTNERNRILHAISKERDLQGTQDDYNKVINI |
| | | IQNLKISDEEVSKALNLDVVFKDKKNIITKINDIKISEENNNDIKYLPS |
| | | FSKVLPEILNLYRNNPKNEPFDTIETEKIVLNALIYVNKELYKKLILE |
| | | DDLEENESKNIFLQELKKTLGNIDEIDENIIENYYKNAQISASKGNNK |
| | | AIKKYQKKVIECYIGYLRKNYEELFDFSDFKMNIQEIKKQIKDINDN |
| | | KTYERITVKTSDKTIVINDDFEYIISIFALLNSNAVINKIRNRFFATSV |
| | | WLNTSEYQNIIDILDEIMQLNTLRNECITENWNLNLEEFIQKMKEIEK |
| | | DFDDFKIQTKKEIFNNYYEDIKNNILTEFKDDINGCDVLEKKLEKIVI |
| | | FDDETKFEIDKKSNILQDEQRKLSNINKKDLKKKVDQYIKDKDQEIK |
| | | SKILCRIIFNSDFLKKYKKEIDNLIEDMESENENKFQEIYYPKERKNEL |
| | | YIYKKNLFLNIGNPNFDKIYGLISNDIKMADAKFLFNIDGKNIRKNKI |
| | | SEIDAILKNLNDKLNGYSKEYKEKYIKKLKENDDFFAKNIQNKNYK |
| | | SFEKDYNRVSEYKKIRDLVEFNYLNKIESYLIDINWKLAIQMARFER |
| | | DMHYIVNGLRELGIIKLSGYNTGISRAYPKRNGSDGFYTTTAYYKFF |
| | | DEESYKKFEKICYGFGIDLSENSEINKPENESIRNYISHFYIVRNPFAD |
| | | YSIAEQIDRVSNLLSYSTRYNNSTYASVFEVFKKDVNLDYDELKKKF |
| | | KLIGNNDILERLMKPKKVSVLELESYNSDYIKNLIIELLTKIENTNDT |
| | | L |
| SEQ | Rhodobacter | MQIGKVQGRTISEFGDPAGGLKRKISTDGKNRKELPAHLSSDPKALI |
| ID | capsulatus | GQWISGIDKIYRKPDSRKSDGKAIHSPTPSKMQFDARDDLGEAFWK |
| NO: | C2c2 amino | LVSEAGLAQDSDYDQFKRRLHPYGDKFQPADSGAKLKFEADPPEPQ |
| 133 | acid sequence | AFHGRWYGAMSKRGNDAKELAAALYEHLHVDEKRIDGQPKRNPK |
| | | TDKFAPGLVVARALGIESSVLPRGMARLARNWGEEEIQTYFVVDVA |
| | | ASVKEVAKAAVSAAQAFDPPRQVSGRSLSPKVGFALAEHLERVTGS |
| | | KRCSFDPAAGPSVLALHDEVKKTYKRLCARGKNAARAFPADKTEL |
| | | LALMRHTHENRVRNQMVRMGRVSEYRGQQAGDLAQSHYWTSAG |
| | | QTEIKESEIFVRLWVGAFALAGRSMKAWIDPMGKIVNTEKNDRDLT |
| | | AAVNIRQVISNKEMVAEAMARRGIYFGETPELDRLGAEGNEGFVFA |
| | | LLRYLRGCRNQTFHLGARAGFLKEIRKELEKTRWGKAKEAEHVVL |
| | | TDKTVAAIRAIIDNDAKALGARLLADLSGAFVAHYASKEHFSTLYSE |
| | | IVKAVKDAPEVSSGLPRLKLLLKRADGVRGYVHGLRDTRKHAFAT |
| | | KLPPPPAPRELDDPATKARYIALLRLYDGPFRAYASGITGTALAGPA |
| | | ARAKEAATALAQSVNVTKAYSDVMEGRSSRLRPPNDGETLREYLS |
| | | ALTGETATEFRVQIGYESDSENARKQAEFIENYRRDMLAFMFEDYIR |
| | | AKGFDWILKIEPGATAMTRAPVLPEPIDTRGQYEHWQAALYLVMH |
| | | FVPASDVSNLLHQLRKWEALQGKYELVQDGDATDQADARREALD |
| | | LVKRFRDVLVLFLKTGEARFEGRAAPFDLKPFRALFANPATFDRLF |
| | | MATPTTARP AEDDPEGDGASEPELRVARTLRGLRQIARYNHMAVLS |
| | | DLFAKHKVRDEEVARLAEIEDETQEKSQIVAAQELRTDLHDKVMK |
| | | CHPKTISPEERQSYAAAIKTIEEHRFLVGRVYLGDHLRLHRLMMDVI |
| | | GRLIDYAGAYERDTGTFLINASKQLGAGADWAVTIAGAANTDART |
| | | QTRKDLAHFNVLDRADGTPDLTALVNRAREMMAYDRKRKNAVPR |
| | | SILDMLARLGLTLKWQMKDHLLQDATITQAAIKHLDKVRLTVGGP |
| | | AAVTEARFSQDYLQMVAAVFNGSVQNPKPRRRDDGDAWHKPPKP |
| | | ATAQSQPDQKPPNKAPSAGSRLPPPQVGEVYEGVVVKVIDTGSLGF |
| | | LAVEGVAGNIGLHISRLRRIREDAIIVGRRYRFRVEIYVPPKSNTSKL |
| | | NAADLVRID |
| SEQ | Carnobacterium | MRITKVKIKLDNKLYQVTMQKEEKYGTLKLNEESRKSTAEILRLKK |
| ID | gallinarum | ASFNKSFHSKTINSQKENKNATIKKNGDYISQIFEKLVGVDTNKNIR |
| NO: | C2c2 amino | KPKMSLTDLKDLPKKDLALFIKRKFKNDDIVEIKNLDLISLFYNALQ |
| 134 | acid sequence | KVPGEHFTDESWADFCQEMMPYREYKNKFIERKIILLANSIEQNKGF |
| | | SINPETFSKRKRVLHQWAIEVQERGDFSILDEKLSKLAEIYNFKKMC |
| | | KRVQDELNDLEKSMKKGKNPEKEKEAYKKQKNFKIKTIWKDYPYK |
| | | THIGLIEKIKENEELNQFNIEIGKYFEHYFPIKKERCTEDEPYYLNSETI |
| | | ATTVNYQLKNALISYLMQIGKYKQFGLENQVLDSKKLQEIGIYEGF |
| | | QTKFMDACVFATSSLKNIIEPMRSGDILGKREFKEAIATSSFVNYHHF |
| | | FPYFPFELKGMKDRESELIPFGEQTEAKQMQNIWALRGSVQQIRNEI |
| | | FHSFDKNQKFNLPQLDKSNFEFDASENSTGKSQSYIETDYKFLFEAE |
| | | KNQLEQFFIERIKSSGALEYYPLKSLEKLFAKKEMKFSLGSQVVAFA |
| | | PSYKKLVKKGHSYQTATEGTANYLGLSYYNRYELKEESFQAQYYL |
| | | LKLIYQYVFLPNFSQGNSPAFRETVKAILRINKDEARKKMKKNKKFL |
| | | RKYAFEQVREMEFKETPDQYMSYLQSEMREEKVRKAEKNDKGFEK |
| | | NITMNFEKLLMQIFVKGFDVFLTTFAGKELLLSSEEKVIKETEISLSK |
| | | KINEREKTLKASIQVEHQLVATNSAISYWLFCKLLDSRHLNELRNEM |
| | | IKFKQSRIKFNHTQHAELIQNLLPIVELTILSNDYDEKNDSQNVDVSA |
| | | YFEDKSLYETAPYVQTDDRTRVSFRPILKLEKYHTKSLIEALLKDNP |
| | | QFRVAATDIQEWMHKREEIGELVEKRKNLHTEWAEGQQTLGAEKR |
| | | EEYRDYCKKIDRFNWKANKVTLTYLSQLHYLITDLLGRMVGFSALF |
| | | ERDLVYFSRSFSELGGETYHISDYKNLSGVLRLNAEVKPIKIKNIKVI |
| | | DNEENPYKGNEPEVKPFLDRLHAYLENVIGIKAVHGKIRNQTAHLS |
| | | VLQLELSMIESMNNLRDLMAYDRKLKNAVTKSMIKILDKHGMILKL |
| | | KIDENHKNFEIESLIPKEIIHLKDKAIKTNQVSEEYCQLVLALLTTNPG |
| | | NQLN |
| SEQ | Herbinix | MKLTRRRISGNSVDQKITAAFYRDMSQGLLYYDSEDNDCTDKVIES |
| ID | hemi- | MDFERSWRGRILKNGEDDKNPFYMFVKGLVGSNDKIVCEPIDVDSD |
| NO: | cellulosilytica | PDNLDILINKNLTGFGRNLKAPDSNDTLENLIRKIQAGIPEEEVLPEL |
| 135 | C2c2 | KKIKEMIQKDIVNRKEQLLKSIKNNRIPFSLEGSKLVPSTKKMKWLF |
| | amino acid | KLIDVPNKTFNEKMLEKYWEIYDYDKLKANITNRLDKTDKKARSIS |
| | sequence | RAVSEELREYHKNLRTNYNRFVSGDRPAAGLDNGGSAKYNPDKEE |
| | | FLLFLKEVEQYFKKYFPVKSKHSNKSKDKSLVDKYKNYCSYKVVK |
| | | KEVNRSIINQLVAGLIQQGKLLYYFYYNDTWQEDFLNSYGLSYIQV |
| | | EEAFKKSVMTSLSWGINRLTSFFIDDSNTVKFDDITTKKAKEAIESNY |
| | | FNKLRTCSRMQDHFKEKLAFFYPVYVKDKKDRPDDDIENLIVLVKN |
| | | AIESVSYLRNRTFHFKESSLLELLKELDDKNSGQNKIDYSVAAEFIKR |
| | | DIENLYDVFREQIRSLGIAEYYKADMISDCFKTCGLEFALYSPKNSL |
| | | MPAFKNVYKRGANLNKAYIRDKGPKETGDQGQNSYKALEEYRELT |
| | | WYIEVKNNDQSYNAYKNLLQLIYYHAFLPEVRENEALITDFINRTKE |
| | | WNRKETEERLNTKNNKKHKNFDENDDITVNTYRYESIPDYQGESLD |
| | | DYLKVLQRKQMARAKEVNEKEEGNNNYIQFIRDVVVWAFGAYLE |
| | | NKLKNYKNELQPPLSKENIGLNDTLKELFPEEKVKSPFNIKCRFSIST |
| | | FIDNKGKSTDNTSAEAVKTDGKEDEKDKKNIKRKDLLCFYLFLRLL |
| | | DENEICKLQHQFIKYRCSLKERRFPGNRTKLEKETELLAELEELMEL |
| | | VRFTMPSIPEISAKAESGYDTMIKKYFKDFIEKKVFKNPKTSNLYYH |
| | | SDSKTPVTRKYMALLMRSAPLHLYKDIFKGYYLITKKECLEYIKLSN |
| | | IIKDYQNSLNELHEQLERIKLKSEKQNGKDSLYLDKKDFYKVKEYV |
| | | ENLEQVARYKHLQHKINFESLYRIFRIHVDIAARMVGYTQDWERDM |
| | | HFLFKALVYNGVLEERRFEAIFNNNDDNNDGRIVKKIQNNLNNKNR |
| | | ELVSMLCWNKKLNKNEFGAIIWKRNPIAHLNHFTQTEQNSKSSLES |
| | | LINSLRILLAYDRKRQNAVTKTINDLLLNDYHIRIKWEGRVDEGQIY |
| | | FNIKEKEDIENEPIIHLKHLHKKDCYIYKNSYMFDKQKEWICNGIKEE |
| | | VYDKSILKCIGNLFKFDYEDKNKSSANPKHT |
| SEQ | Paludibacter | MRVSKVKVKDGGKDKMVLVHRKTTGAQLVYSGQPVSNETSNILPE |
| ID | propionicigenes | KKRQSFDLSTLNKTIIKFDTAKKQKLNVDQYKIVEKIFKYPKQELPK |
| NO: | C2c2 amino | QIKAEEILPFLNHKFQEPVKYWKNGKEESFNLTLLIVEAVQAQDKR |
| 136 | acid sequence | KLQPYYDWKTWYIQTKSDLLKKSIENNRIDLTENLSKRKKALLAWE |
| | | TEFTASGSIDLTHYHKVYMTDVLCKMLQDVKPLTDDKGKINTNAY |
| | | HRGLKKALQNHQPAIFGTREVPNEANRADNQLSIYHLEVVKYLEHY |
| | | FPIKTSKRRNTADDIAHYLKAQTLKTTIEKQLVNAIRANIIQQGKTNH |
| | | HELKADTTSNDLIRIKTNEAFVLNLTGTCAFAANNIRNMVDNEQTN |
| | | DILGKGDFIKSLLKDNTNSQLYSFFFGEGLSTNKAEKETQLWGIRGA |
| | | VQQIRNNVNHYKKDALKTVFNISNFENPTITDPKQQTNYADTIYKA |
| | | RFINELEKIPEAFAQQLKTGGAVSYYTIENLKSLLTTFQFSLCRSTIPF |
| | | APGFKKVFNGGINYQNAKQDESFYELMLEQYLRKENFAEESYNAR |
| | | YFMLKLIYNNLFLPGFTTDRKAFADSVGFVQMQNKKQAEKVNPRK |
| | | KEAYAFEAVRPMTAADSIADYMAYVQSELMQEQNKKEEKVAEET |
| | | RINFEKFVLQVFIKGFDSFLRAKEFDFVQMPQPQLTATASNQQKAD |
| | | KLNQLEASITADCKLTPQYAKADDATHIAFYVFCKLLDAAHLSNLR |
| | | NELIKFRESVNEFKFHHLLEIIEICLLSADVVPTDYRDLYSSEADCLA |
| | | RLRPFIEQGADITNWSDLFVQSDKHSPVIHANIELSVKYGTTKLLEQI |
| | | INKDTQFKTTEANFTAWNTAQKSIEQLIKQREDHHEQWVKAKNAD |
| | | DKEKQERKREKSNFAQKFIEKHGDDYLDICDYINTYNWLDNKMHF |
| | | VHLNRLHGLTIELLGRMAGFVALFDRDFQFFDEQQIADEFKLHGFV |
| | | NLHSIDKKLNEVPTKKIKEIYDIRNKIIQINGNKINESVRANLIQFISSK |
| | | RNYYNNAFLHVSNDEIKEKQMYDIRNHIAHFNYLTKDAADFSLIDLI |
| | | NELRELLHYDRKLKNAVSKAFIDLFDKHGMILKLKLNADHKLKVES |
| | | LEPKKIYHLGSSAKDKPEYQYCTNQVMMAYCNMCRSLLEMKK |
| SEQ | Leptotrichia | MYMKITKIDGVSHYKKQDKGILKKKWKDLDERKQREKIEARYNKQ |
| ID | wadei (Lwa) | IESKIYKEFFRLKNKKRIEKEEDQNIKSLYFFIKELYLNEKNEEWELK |
| NO: | C2c2 amino | NINLEILDDKERVIKGYKFKEDVYFFKEGYKEYYLRILFNNLIEKVQ |
| 137 | acid sequence | NENREKVRKNKEFLDLKEIFKKYKNRKIDLLLKSINNNKINLEYKKE |
| | | NVNEEIYGINPTNDREMTFYELLKEIIEKKDEQKSILEEKLDNFDITNF |
| | | LENIEKIFNEETEINIIKGKVLNELREYIKEKEENNSDNKLKQIYNLEL |
| | | KKYIENNFSYKKQKSKSKNGKNDYLYLNFLKKIMFIEEVDEKKEIN |
| | | KEKFKNKINSNFKNLFVQHILDYGKLLYYKENDEYIKNTGQLETKD |
| | | LEYIKTKETLIRKMAVLVSFAANSYYNLFGRVSGDILGTEVVKSSKT |
| | | NVIKVGSHIFKEKMLNYFFDFEIFDANKIVEILESISYSIYNVRNGVG |
| | | HFNKLILGKYKKKDINTNKRIEEDLNNNEEIKGYFIKKRGEIERKVK |
| | | EKFLSNNLQYYYSKEKIENYFEVYEFEILKRKIPFAPNFKRIIKKGED |
| | | LFNNKNNKKYEYFKNFDKNSAEEKKEFLKTRNFLLKELYYNNFYK |
| | | EFLSKKEEFEKIVLEVKEEKKSRGNINNKKSGVSFQSIDDYDTKINIS |
| | | DYIASIHKKEMERVEKYNEEKQKDTAKYIRDFVEEIFLTGFINYLEK |
| | | DKRLHFLKEEFSILCNNNNNVVDFNININEEKIKEFLKENDSKTLNLY |
| | | LFFNMIDSKRISEFRNELVKYKQFTKKRLDEEKEFLGIKIELYETLIEF |
| | | VILTREKLDTKKSEEIDAWLVDKLYVKDSNEYKEYEEILKLFVDEKI |
| | | LSSKEAPYYATDNKTPILLSNFEKTRKYGTQSFLSEIQSNYKYSKVE |
| | | KENIEDYNKKEEIEQKKKSNIEKLQDLKVELHKKWEQNKITEKEIEK |
| | | YNNTTRKINEYNYLKNKEELQNVYLLHEMLSDLLARNVAFFNKWE |
| | | RDFKFIVIAIKQFLRENDKEKVNEFLNPPDNSKGKKVYFSVSKYKNT |
| | | VENIDGIHKNFMNLIFLNNKFMNRKIDKMNCAIWVYFRNYIAHFLH |
| | | LHTKNEKISLISQMNLLIKLFSYDKKVQNHILKSTKTLLEKYNIQINF |
| | | EISNDKNEVFKYKIKNRLYSKKGKMLGKNNKFEILENEFLENVKAM |
| | | LEYSE |
| SEQ | Bergeyella | MENKTSLGNNIYYNPFKPQDKSYFAGYFNAAMENTDSVFRELGKR |
| ID | zoohelcum | LKGKEYTSENFFDAIFKENISLVEYERYVKLLSDYFPMARLLDKKEV |
| NO: | Cas13b | PIKERKENFKKNFKGIIKAVRDLRNFYTHKEHGEVEITDEIFGVLDE |
| 138 | | MLKSTVLTVKKKKVKTDKTKEILKKSIEKQLDILCQKKLEYLRDTA |
| | | RKIEEKRRNQRERGEKELVAPFKYSDKRDDLIAAIYNDAFDVYIDK |
| | | KKDSLKESSKAKYNTKSDPQQEEGDLKIPISKNGVVFLLSLFLTKQEI |
| | | HAFKSKIAGFKATVIDEATVSEATVSHGKNSICFMATHEIFSHLAYK |
| | | KLKRKVRTAEINYGEAENAEQLSVYAKETLMMQMLDELSKVPDVV |
| | | YQNLSEDVQKTFIEDWNEYLKENNGDVGTMEEEQVIHPVIRKRYED |
| | | KFNYFAIRFLDEFAQFPTLRFQVHLGNYLHDSRPKENLISDRRIKEKI |
| | | TVFGRLSELEHKKALFIKNTETNEDREHYWEIFPNPNYDFPKENISV |
| | | NDKDFPIAGSILDREKQPVAGKIGIKVKLLNQQYVSEVDKAVKAHQ |
| | | LKQRKASKPSIQNIIEEIVPINESNPKEAIVFGGQPTAYLSMNDIHSILY |
| | | EFFDKWEKKKEKLEKKGEKELRKEIGKELEKKIVGKIQAQIQQIIDK |
| | | DTNAKILKPYQDGNSTAIDKEKLIKDLKQEQNILQKLKDEQTVREKE |
| | | YNDFIAYQDKNREINKVRDRNHKQYLKDNLKRKYPEAPARKEVLY |
| | | YREKGKVAVWLANDIKRFMPTDFKNEWKGEQHSLLQKSLAYYEQ |
| | | CKEELKNLLPEKVFQHLPFKLGGYFQQKYLYQFYTCYLDKRLEYIS |
| | | GLVQQAENFKSENKVFKKVENECFKFLKKQNYTHKELDARVQSIL |
| | | GYPIFLERGFMDEKPTIIKGKTFKGNEALFADWFRYYKEYQNFQTFY |
| | | DTENYPLVELEKKQADRKRKTKIYQQKKNDVFTLLMAKHIFKSVFK |
| | | QDSIDQFSLEDLYQSREERLGNQERARQTGERNTNYIWNKTVDLKL |
| | | CDGKITVENVKLKNVGDFIKYEYDQRVQAFLKYEENIEWQAFLIKE |
| | | SKEEENYPYVVEREIEQYEKVRREELLKEVHLIEEYILEKVKDKEILK |
| | | KGDNQNFKYYILNGLLKQLKNEDVESYKVFNLNTEPEDVNINQLKQ |
| | | EATDLEQKAFVLTYIRNKFAHNQLPKKEFWDYCQEKYGKIEKEKTY |
| | | AEYFAEVFKKEKEALIK |
| SEQ | Prevotella | MEDDKKTTDSIRYELKDKHFWAAFLNLARHNVYITVNHINKILEEG |
| ID | intermedia | EINRDGYETTLKNTWNEIKDINKKDRLSKLIIKHFPFLEAATYRLNPT |
| NO: | Cas13b | DTTKQKEEKQAEAQSLESLRKSFFVFIYKLRDLRNHYSHYKHSKSLE |
| 139 | | RPKFEEGLLEKMYNIFNASIRLVKEDYQYNKDINPDEDFKHLDRTEE |
| | | EFNYYFTKDNEGNITESGLLFFVSLFLEKKDAIWMQQKLRGFKDNR |
| | | ENKKKMTNEVFCRSRMLLPKLRLQSTQTQDWILLDMLNELIRCPKS |
| | | LYERLREEDREKFRVPIEIADEDYDAEQEPFKNTLVRHQDRFPYFAL |
| | | RYFDYNEIFTNLRFQIDLGTYHFSIYKKQIGDYKESHHLTHKLYGFE |
| | | RIQEFTKQNRPDEWRKFVKTFNSFETSKEPYIPETTPHYHLENQKIGI |
| | | RFRNDNDKIWPSLKTNSEKNEKSKYKLDKSFQAEAFLSVHELLPMM |
| | | FYYLLLKTENTDNDNEIETKKKENKNDKQEKHKIEEIIENKITEIYAL |
| | | YDTFANGEIKSIDELEEYCKGKDIEIGHLPKQMIAILKDEHKVMATE |
| | | AERKQEEMLVDVQKSLESLDNQINEEIENVERKNSSLKSGKIASWL |
| | | VNDMMRFQPVQKDNEGKPLNNSKANSTEYQLLQRTLAFFGSEHER |
| | | LAPYFKQTKLIESSNPHPFLKDTEWEKCNNILSFYRSYLEAKKNFLES |
| | | LKPEDWEKNQYFLKLKEPKTKPKTLVQGWKNGFNLPRGIFTEPIRK |
| | | WFMKHRENITVAELKRVGLVAKVIPLFFSEEYKDSVQPFYNYHFNV |
| | | GNINKPDEKNFLNCEERRELLRKKKDEFKKMTDKEKEENPSYLEFK |
| | | SWNKFERELRLVRNQDIVTWLLCMELFNKKKIKELNVEKIYLKNIN |
| | | TNTTKKEKNTEEKNGEEKNIKEKNNILNRIMPMRLPIKVYGRENFSK |
| | | NKKKKIRRNTFFTVYIEEKGTKLLKQGNFKALERDRRLGGLFSFVKT |
| | | PSKAESKSNTISKLRVEYELGEYQKARIEIIKDMLALEKTLIDKYNSL |
| | | DTDNFNKMLTDWLELKGEPDKASFQNDVDLLIAVRNAFSHNQYPM |
| | | RNRIAFANINPFSLSSANTSEEKGLGIANQLKDKTHKTIEKIIEIEKPIE |
| | | TKE |
| SEQ | Prevotella | MQKQDKLFVDRKKNAIFAFPKYITIMENKEKPEPIYYELTDKHFWA |
| ID | buccae Cas13b | AFLNLARHNVYTTINHINRRLEIAELKDDGYMMGIKGSWNEQAKK |
| NO: | | LDKKVRLRDLIMKHFPFLEAAAYEMTNSKSPNNKEQREKEQSEALS |
| 140 | | LNNLKNVLFIFLEKLQVLRNYYSHYKYSEESPKPIFETSLLKNMYKV |
| | | FDANVRLVKRDYMHHENIDMQRDFTHLNRKKQVGRTKNIIDSPNF |
| | | HYHFADKEGNMTIAGLLFFVSLFLDKKDAIWMQKKLKGFKDGRNL |
| | | REQMTNEVFCRSRISLPKLKLENVQTKDWMQLDMLNELVRCPKSL |
| | | YERLREKDRESFKVPFDIFSDDYNAEEEPFKNTLVRHQDRFPYFVLR |
| | | YFDLNEIFEQLRFQIDLGTYHFSIYNKRIGDEDEVRHLTHHLYGFARI |
| | | QDFAPQNQPEEWRKLVKDLDHFETSQEPYISKTAPHYHLENEKIGIK |
| | | FCSAHNNLFPSLQTDKTCNGRSKFNLGTQFTAEAFLSVHELLPMMF |
| | | YYLLLTKDYSRKESADKVEGIIRKEISNIYAIYDAFANNEINSIADLTR |
| | | RLQNTNILQGHLPKQMISILKGRQKDMGKEAERKIGEMIDDTQRRL |
| | | DLLCKQTNQKIRIGKRNAGLLKSGKIADWLVNDMMRFQPVQKDQN |
| | | NIPINNSKANSTEYRMLQRALALFGSENFRLKAYFNQMNLVGNDNP |
| | | HPFLAETQWEHQTNILSFYRNYLEARKKYLKGLKPQNWKQYQHFLI |
| | | LKVQKTNRNTLVTGWKNSFNLPRGIFTQPIREWFEKHNNSKRIYDQI |
| | | LSFDRVGFVAKAIPLYFAEEYKDNVQPFYDYPFNIGNRLKPKKRQFL |
| | | DKKERVELWQKNKELFKNYPSEKKKTDLAYLDFLSWKKFERELRLI |
| | | KNQDIVTWLMFKELFNMATVEGLKIGEIHLRDIDTNTANEESNNILN |
| | | RIMPMKLPVKTYETDNKGNILKERPLATFYIEETETKVLKQGNFKAL |
| | | VKDRRLNGLFSFAETTDLNLEEHPISKLSVDLELIKYQTTRISIFEMTL |
| | | GLEKKLIDKYSTLPTDSFRNMLERWLQCKANRPELKNYVNSLIAVR |
| | | NAFSHNQYPMYDATLFAEVKKFTLFPSVDTKKIELNIAPQLLEIVGK |
| | | AIKEIEKSENKN |
| SEQ | Porphyromonas | MNTVPASENKGQSRTVEDDPQYFGLYLNLARENLIEVESHVRIKFG |
| ID | gingivalis | KKKLNEESLKQSLLCDHLLSVDRWTKVYGHSRRYLPFLHYFDPDSQ |
| NO: | Cas13b | IEKDHDSKTGVDPDSAQRLIRELYSLLDFLRNDFSHNRLDGTTFEHL |
| 141 | | EVSPDISSFITGTYSLACGRAQSRFAVFFKPDDFVLAKNRKEQLISVA |
| | | DGKECLTVSGFAFFICLFLDREQASGMLSRIRGFKRTDENWARAVH |
| | | ETFCDLCIRHPHDRLESSNTKEALLLDMLNELNRCPRILYDMLPEEE |
| | | RAQFLPALDENSMNNLSENSLDEESRLLWDGSSDWAEALTKRIRHQ |
| | | DRFPYLMLRFIEEMDLLKGIRFRVDLGEIELDSYSKKVGRNGEYDRT |
| | | ITDHALAFGKLSDFQNEEEVSRMISGEASYPVRFSLFAPRYAIYDNKI |
| | | GYCHTSDPVYPKSKTGEKRALSNPQSMGFISVHDLRKLLLMELLCE |
| | | GSFSRMQSDFLRKANRILDETAEGKLQFSALFPEMRHRFIPPQNPKS |
| | | KDRREKAETTLEKYKQEIKGRKDKLNSQLLSAFDMDQRQLPSRLLD |
| | | EWMNIRPASHSVKLRTYVKQLNEDCRLRLRKFRKDGDGKARAIPL |
| | | VGEMATFLSQDIVRMIISEETKKLITSAYYNEMQRSLAQYAGEENRR |
| | | QFRAIVAELRLLDPSSGHPFLSATMETAHRYTEGFYKCYLEKKREW |
| | | LAKIFYRPEQDENTKRRISVFFVPDGEARKLLPLLIRRRMKEQNDLQ |
| | | DWIRNKQAHPIDLPSHLFDSKVMELLKVKDGKKKWNEAFKDWWS |
| | | TKYPDGMQPFYGLRRELNIHGKSVSYIPSDGKKFADCYTHLMEKTV |
| | | RDKKRELRTAGKPVPPDLAADIKRSFHRAVNEREFMLRLVQEDDRL |
| | | MLMAINKMMTDREEDILPGLKNIDSILDEENQFSLAVHAKVLEKEG |
| | | EGGDNSLSLVPATIEIKSKRKDWSKYIRYRYDRRVPGLMSHFPEHK |
| | | ATLDEVKTLLGEYDRCRIKIFDWAFALEGAIMSDRDLKPYLHESSSR |
| | | EGKSGEHSTLVKMLVEKKGCLTPDESQYLILIRNKAAHNQFPCAAE |
| | | MPLIYRDVSAKVGSIEGSSAKDLPEGSSLVDSLWKKYEMIIRKILPIL |
| | | DPENRFFGKLLNNMSQPINDL |
| SEQ | Bacteroides | MESIKNSQKSTGKTLQKDPPYFGLYLNMALLNVRKVENHIRKWLG |
| ID | pyogenes | DVALLPEKSGFHSLLTTDNLSSAKWTRFYYKSRKFLPFLEMFDSDK |
| NO: | Cas13b | KSYENRRETAECLDTIDRQKISSLLKEVYGKLQDIRNAFSHYHIDDQ |
| 142 | | SVKHTALIISSEMHRFIENAYSFALQKTRARFTGVFVETDFLQAEEK |
| | | GDNKKFFAIGGNEGIKLKDNALIFLICLFLDREEAFKFLSRATGFKST |
| | | KEKGFLAVRETFCALCCRQPHERLLSVNPREALLMDMLNELNRCPD |
| | | ILFEMLDEKDQKSFLPLLGEEEQAHILENSLNDELCEAIDDPFEMIAS |
| | | LSKRVRYKNRFPYLMLRYIEEKNLLPFIRFRIDLGCLELASYPKKMG |
| | | EENNYERSVTDHAMAFGRLTDFHNEDAVLQQITKGITDEVRFSLYA |
| | | PRYAIYNNKIGFVRTSGSDKISFPTLKKKGGEGHCVAYTLQNTKSFG |
| | | FISIYDLRKILLLSFLDKDKAKNIVSGLLEQCEKHWKDLSENLFDAIR |
| | | TELQKEFPVPLIRYTLPRSKGGKLVSSKLADKQEKYESEFERRKEKL |
| | | TEILSEKDFDLSQIPRRMIDEWLNVLPTSREKKLKGYVETLKLDCRE |
| | | RLRVFEKREKGEHPLPPRIGEMATDLAKDIIRMVIDQGVKQRITSAY |
| | | YSEIQRCLAQYAGDDNRRHLDSIIRELRLKDTKNGHPFLGKVLRPGL |
| | | GHTEKLYQRYFEEKKEWLEATFYPAASPKRVPRFVNPPTGKQKELP |
| | | LIIRNLMKERPEWRDWKQRKNSHPIDLPSQLFENEICRLLKDKIGKE |
| | | PSGKLKWNEMFKLYWDKEFPNGMQRFYRCKRRVEVFDKVVEYEY |
| | | SEEGGNYKKYYEALIDEVVRQKISSSKEKSKLQVEDLTLSVRRVFKR |
| | | AINEKEYQLRLLCEDDRLLFMAVRDLYDWKEAQLDLDKIDNMLGE |
| | | PVSVSQVIQLEGGQPDAVIKAECKLKDVSKLMRYCYDGRVKGLMP |
| | | YFANHEATQEQVEMELRHYEDHRRRVFNWVFALEKSVLKNEKLRR |
| | | FYEESQGGCEHRRCIDALRKASLVSEEEYEFLVHIRNKSAHNQFPDL |
| | | EIGKLPPNVTSGFCECIWSKYKAIICRIIPFIDPERRFFGKLLEQK |
| SEQ | Cas13c | MTEKKSIIFKNKSSVEIVKKDIFSQTPDNMIRNYKITLKISEKNPRVVE |
| ID | | AEIEDLMNSTILKDGRRSARREKSMTERKLIEEKVAENYSLLANCPM |
| NO: | | EEVDSIKIYKIKRFLTYRSNMLLYFASINSFLCEGIKGKDNETEEIWH |
| 143 | | LKDNDVRKEKVKENFKNKLIQSTENYNSSLKNQIEEKEKLLRKESK |
| | | KGAFYRTIIKKLQQERIKELSEKSLTEDCEKIIKLYSELRHPLMHYDY |
| | | QYFENLFENKENSELTKNLNLDIFKSLPLVRKMKLNNKVNYLEDND |
| | | TLFVLQKTKKAKTLYQIYDALCEQKNGFNKFINDFFVSDGEENTVF |
| | | KQIINEKFQSEMEFLEKRISESEKKNEKLKKKFDSMKAHFHNINSED |
| | | TKEAYFWDIHSSSNYKTKYNERKNLVNEYTELLGSSKEKKLLREEIT |
| | | QINRKLLKLKQEMEEITKKNSLFRLEYKMKIAFGFLFCEFDGNISKF |
| | | KDEFDASNQEKIIQYHKNGEKYLTYFLKEEEKEKFNLEKMQKIIQKT |
| | | EEEDWLLPETKNNLFKFYLLTYLLLPYELKGDFLGFVKKHYYDIKN |
| | | VDFMDENQNNIQVSQTVEKQEDYFYHKIRLFEKNTKKYEIVKYSIV |
| | | PNEKLKQYFEDLGIDIKYLTGSVESGEKWLGENLGIDIKYLTVEQKS |
| | | EVSEEKIKKFL |
| SEQ | Cas13c | MEKDKKGEKIDISQEMIEEDLRKILILFSRLRHSMVHYDYEFYQALY |
| ID | | SGKDFVISDKNNLENRMISQLLDLNIFKELSKVKLIKDKAISNYLDK |
| NO: | | NTTIHVLGQDIKAIRLLDIYRDICGSKNGFNKFINTMITISGEEDREYK |
| 144 | | EKVIEHFNKKMENLSTYLEKLEKQDNAKRNNKRVYNLLKQKLIEQ |
| | | QKLKEWFGGPYVYDIHSSKRYKELYIERKKLVDRHSKLFEEGLDEK |
| | | NKKELTKINDELSKLNSEMKEMTKLNSKYRLQYKLQLAFGFILEEF |
| | | DLNIDTFINNFDKDKDLIISNFMKKRDIYLNRVLDRGDNRLKNIIKEY |
| | | KFRDTEDIFCNDRDNNLVKLYILMYILLPVEIRGDFLGFVKKNYYD |
| | | MKHVDFIDKKDKEDKDTFFHDLRLFEKNIRKLEITDYSLSSGFLSKE |
| | | HKVDIEKKINDFINRNGAMKLPEDITIEEFNKSLILPIMKNYQINFKLL |
| | | NDIEISALFKIAKDRSITFKQAIDEIKNEDIKKNSKKNDKNNHKDKNI |
| | | NFTQLMKRALHEKIPYKAGMYQIRNNISHIDMEQLYIDPLNSYMNS |
| | | NKNNITISEQIEKIIDVCVTGGVTGKELNNNIINDYYMKKEKLVFNL |
| | | KLRKQNDIVSIESQEKNKREEFVFKKYGLDYKDGEINIIEVIQKVNSL |
| | | QEELRNIKETSKEKLKNKETLFRDISLINGTIRKNINFKIKEMVLDIVR |
| | | MDEIRHINIHIYYKGENYTRSNIIKFKYAIDGENKKYYLKQHEINDIN |
| | | LELKDKFVTLICNMDKHPNKNKQTINLESNYIQNVKFIIP |
| SEQ | Cas13c | MENKGNNKKIDFDENYNILVAQIKEYFTKEIENYNNRIDNIIDKKEL |
| ID | | LKYSEKKEESEKNKKLEELNKLKSQKLKILTDEEIKADVIKIIKIFSDL |
| NO: | | RHSLMHYEYKYFENLFENKKNEELAELLNLNLFKNLTLLRQMKIEN |
| 145 | | KTNYLEGREEFNIIGKNIKAKEVLGHYNLLAEQKNGFNNFINSFFVQ |
| | | DGTENLEFKKLIDEHFVNAKKRLERNIKKSKKLEKELEKMEQHYQR |
| | | LNCAYVWDIHTSTTYKKLYNKRKSLIEEYNKQINEIKDKEVITAINV |
| | | ELLRIKKEMEEITKSNSLFRLKYKMQIAYAFLEIEFGGNIAKFKDEFD |
| | | CSKMEEVQKYLKKGVKYLKYYKDKEAQKNYEFPFEEIFENKDTHN |
| | | EEWLENTSENNLFKFYILTYLLLPMEFKGDFLGVVKKHYYDIKNVD |
| | | FTDESEKELSQVQLDKMIGDSFFHKIRLFEKNTKRYEIIKYSILTSDEI |
| | | KRYFRLLELDVPYFEYEKGTDEIGIFNKNIILTIFKYYQIIFRLYNDLEI |
| | | HGLFNISSDLDKILRDLKSYGNKNINFREFLYVIKQNNNSSTEEEYRK |
| | | IWENLEAKYLRLHLLTPEKEEIKTKTKEELEKLNEISNLRNGICHLNY |
| | | KEIIEEILKTEISEKNKEATLNEKIRKVINFIKENELDKVELGFNFINDF |
| | | FMKKEQFMFGQIKQVKEGNSDSITTERERKEKNNKKLKETYELNCD |
| | | NLSEFYETSNNLRERANSSSLLEDSAFLKKIGLYKVKNNKVNSKVK |
| | | DEEKRIENIKRKLLKDSSDIMGMYKAEVVKKLKEKLILIFKHDEEKR |
| | | IYVTVYDTSKAVPENISKEILVKRNNSKEEYFFEDNNKKYVTEYYTL |
| | | EITETNELKVIPAKKLEGKEFKTEKNKENKLMLNNHYCFNVKIIY |
| SEQ | Cas13c | MEEIKHKKNKSSIIRVIVSNYDMTGIKEIKVLYQKQGGVDTFNLKTII |
| ID | | NLESGNLEIISCKPKEREKYRYEFNCKTEINTISITKKDKVLKKEIRKY |
| NO: | | SLELYFKNEKKDTVVAKVTDLLKAPDKIEGERNHLRKLSSSTERKL |
| 146 | | LSKTLCKNYSEISKTPIEEIDSIKIYKIKRFLNYRSNFLIYFALINDFLC |
| | | AGVKEDDINEVWLIQDKEHTAFLENRIEKITDYIFDKLSKDIENKKN |
| | | QFEKRIKKYKTSLEELKTETLEKNKTFYIDSIKTKITNLENKITELSLY |
| | | NSKESLKEDLIKIISIFTNLRHSLMHYDYKSFENLFENIENEELKNLLD |
| | | LNLFKSIRMSDEFKTKNRTNYLDGTESFTIVKKHQNLKKLYTYYNN |
| | | LCDKKNGFNTFINSFFVTDGIENTDFKNLIILHFEKEMEEYKKSIEYY |
| | | KIKISNEKNKSKKEKLKEKIDLLQSELINMREHKNLLKQIYFFDIHNSI |
| | | KYKELYSERKNLIEQYNLQINGVKDVTAINHINTKLLSLKNKMDKIT |
| | | KQNSLYRLKYKLKIAYSFLMIEFDGDVSKFKNNFDPTNLEKRVEYL |
| | | DKKEEYLNYTAPKNKFNFAKLEEELQKIQSTSEMGADYLNVSPENN |
| | | LFKFYILTYIMLPVEFKGDFLGFVKNHYYNIKNVDFMDESLLDENEV |
| | | DSNKLNEKIENLKDSSFFNKIRLFEKNIKKYEIVKYSVSTQENMKEY |
| | | FKQLNLDIPYLDYKSTDEIGIFNKNMILPIFKYYQNVFKLCNDIEIHA |
| | | LLALANKKQQNLEYAIYCCSKKNSLNYNELLKTFNRKTYQNLSFIR |
| | | NKIAHLNYKELFSDLFNNELDLNTKVRCLIEFSQNNKFDQIDLGMNF |
| | | INDYYMKKTRFIFNQRRLRDLNVPSKEKIIDGKRKQQNDSNNELLK |
| | | KYGLSRTNIKDIFNKAWY |
| SEQ | Cas13c | MKVRYRKQAQLDTFIIKTEIVNNDIFIKSIIEKAREKYRYSFLFDGEE |
| ID | | KYHFKNKSSVEIVKNDIFSQTPDNMIRNYKITLKISEKNPRVVEAEIE |
| NO: | | DLMNSTILKDGRRSARREKSMTERKLIEEKVAENYSLLANCPIEEVD |
| 147 | | SIKIYKIKRFLTYRSNMLLYFASINSFLCEGIKGKDNETEEIWHLKDN |
| | | DVRKEKVKENFKNKLIQSTENYNSSLKNQIEEKEKLSSKEFKKGAFY |
| | | RTIIKKLQQERIKELSEKSLTEDCEKIIKLYSELRHPLMHYDYQYFEN |
| | | LFENKENSELTKNLNLDIFKSLPLVRKMKLNNKVNYLEDNDTLFVL |
| | | QKTKKAKTLYQIYDALCEQKNGFNKFINDFFVSDGEENTVFKQIINE |
| | | KFQSEMEFLEKRISESEKKNEKLKKKLDSMKAHFRNINSEDTKEAYF |
| | | WDIHSSRNYKTKYNERKNLVNEYTKLLGSSKEKKLLREEITKINRQL |
| | | LKLKQEMEEITKKNSLFRLEYKMKIAFGFLFCEFDGNISKFKDEFDA |
| | | SNQEKIIQYHKNGEKYLTSFLKEEEKEKFNLEKMQKIIQKTEEEDWL |
| | | LPETKNNLFKFYLLTYLLLPYELKGDFLGFVKKHYYDIKNVDFMDE |
| | | NQNNIQVSQTVEKQEDYFYHKIRLFEKNTKKYEIVKYSIVPNEKLKQ |
| | | YFEDLGIDIKYLTGSVESGEKWLGENLGIDIKYLTVEQKSEVSEEKN |
| | | KKVSLKNNGMFNKTILLFVFKYYQIAFKLFNDIELYSLFFLREKSEKP |
| | | FEVFLEELKDKMIGKQLNFGQLLYVVYEVLVKNKDLDKILSKKIDY |
| | | RKDKSFSPEIAYLRNFLSHLNYSKFLDNFMKINTNKSDENKEVLIPSI |
| | | KIQKMIQFIEKCNLQNQIDFDFNFVNDFYMRKEKMFFIQLKQIFPDIN |
| | | STEKQKKSEKEEILRKRYHLINKKNEQIKDEHEAQSQLYEKILSLQKI |
| | | FSCDKNNFYRRLKEEKLLFLEKQGKKKISMKEIKDKIASDISDLLGIL |
| | | KKEITRDIKDKLTEKFRYCEEKLLNISFYNHQDKKKEEGIRVFLIRDK |
| | | NSDNFKFESILDDGSNKIFISKNGKEITIQCCDKVLETLMIEKNTLKIS |
| | | SNGKIISLIPHYSYSIDVKY |

The programmable nuclease can be Cas13. Sometimes the Cas13 can be Cas13a, Cas13b, Cas13c, Cas13d, or Cas13e. In some cases, the programmable nuclease can be Mad7 or Mad2. In some cases, the programmable nuclease can be Cas12. Sometimes the Cas12 can be Cas12a, Cas12b, Cas12c, Cas12d, or Cas12e. In some cases, the programmable nuclease can be Csm1, Cas9, C2c4, C2c8, C2c5, C2c10, C2c9, or CasZ. Sometimes, the Csm1 can also be also called smCms1, miCms1, obCms1, or suCms1. Sometimes Cas13a can also be also called C2c2. Sometimes CasZ can also be called Cas14a, Cas14b, Cas14c, Cas14d, Cas14e, Cas14f, Cas14g, or Cas14h. Sometimes, the programmable nuclease can be a type V CRISPR-Cas system. In some cases, the programmable nuclease can be a type VI CRISPR-Cas system. Sometimes the programmable nuclease can be a type III CRISPR-Cas system. In some cases, the programmable nuclease can be from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pint), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Therms thermophilus (Tt). Sometimes the Cas13 is at least one of LbuCas13a, LwaCas13a, LbaCas13a, HheCas13a, PprCas13a, EreCas13a, CamCas13a, or LshCas13a. The trans cleavage activity of the CRISPR enzyme can be activated when the crRNA is complexed with the target nucleic acid. The trans cleavage activity of the CRISPR enzyme can be activated when the guide nucleic acid comprising a tracrRNA and crRNA are complexed with the target nucleic acid. The target nucleic acid can be RNA or DNA.

In some embodiments, a programmable nuclease as disclosed herein is an RNA-activated programmable RNA nuclease. In some embodiments, a programmable nuclease as disclosed herein is a DNA-activated programmable RNA nuclease. In some embodiments, a programmable nuclease is capable of being activated by a target RNA to initiate trans cleavage of an RNA reporter and is capable of being activated by a target DNA to initiate trans cleavage of an RNA reporter, such as a Type VI CRISPR/Cas enzyme (e.g., Cas13). For example, Cas13a of the present disclosure can be activated by a target RNA to initiate trans cleavage activity of the Cas13a for the cleavage of an RNA reporter and can be activated by a target DNA to initiate trans cleavage activity of the Cas13a for trans cleavage of an RNA reporter. An RNA reporter can be an RNA-based reporter molecule. In some embodiments, the Cas13a recognizes and detects ssDNA to initiate transcleavage of RNA reporters. Multiple Cas13a isolates can recognize, be activated by, and detect target DNA, including ssDNA, upon hybridization of a guide nucleic acid with the target DNA. For example, LbuCas13a and LwaCas13a can both be activated to transcollaterally cleave RNA reporters by target DNA. Thus, Type VI CRISPR/Cas enzyme (e.g., Cas13, such as Cas13a) can be DNA-activated programmable RNA nucleases, and therefore, can be used to detect a target DNA using the methods as described herein. DNA-activated programmable RNA nuclease detection of ssDNA can be robust at multiple pH values. For example, target ssDNA detection by Cas13 can exhibit consistent cleavage across a wide range of pH conditions, such as from a pH of 6.8 to a pH of 8.2. In contrast, target RNA detection by Cas13 may exhibit high cleavage activity of pH values from 7.9 to 8.2. In some embodiments, a DNA-activated programmable RNA nuclease that also is capable of being an RNA-activated programmable RNA nuclease, can have DNA targeting preferences that are distinct from its RNA targeting preferences. For example, the optimal ssDNA targets for Cas13a have different properties than optimal RNA targets for Cas13a. As one example, gRNA performance on ssDNA may not necessarily correlate with the performance of the same gRNAs on RNA. As another example, gRNAs can perform at a high level regardless of target nucleotide identity at a 3′ position on a target RNA sequence. In some embodiments, gRNAs can perform at a high level in the absence of a G at a 3′ position on a target ssDNA sequence. Furthermore, target DNA detected by Cas13 disclosed herein can be directly from organisms, or can be indirectly generated by nucleic acid amplification methods, such as PCR and LAMP or any amplification method described herein. Key steps for the sensitive detection of a target DNA, such as a target ssDNA, by a DNA-activated programmable RNA nuclease, such as Cas13a, can include: (1) production or isolation of DNA to concentrations above about 0.1 nM per reaction for in vitro diagnostics, (2) selection of a target sequence with the appropriate sequence features to enable DNA detection as these features are distinct from those required for RNA detection, and (3) buffer composition that enhances DNA detection. The detection of a target DNA by a DNA-activated programmable RNA nuclease can be connected to a variety of readouts including fluorescence, lateral flow, electrochemistry, or any other readouts described herein. Multiplexing of programmable DNA nuclease, such as a Type V CRISPR-Cas protein, with a DNA-activated programmable RNA nuclease, such as a Type VI protein, with a DNA reporter and an RNA reporter, can enable multiplexed detection of target ssDNAs or a combination of a target dsDNA and a target ssDNA, respectively. Multiplexing of different RNA-activated programmable RNA nucleases that have distinct RNA reporter cleavage preferences can enable additional multiplexing. Methods for the generation of ssDNA for DNA-activated programmable RNA nuclease-based diagnostics can include (1) asymmetric PCR, (2) asymmetric isothermal amplification, such as RPA, LAMP, SDA, etc. (3) NEAR for the production of short ssDNA molecules, and (4) conversion of RNA targets into ssDNA by a reverse transcriptase followed by RNase H digestion. Thus, DNA-activated programmable RNA nuclease detection of target DNA is compatible with the various systems, kits, compositions, reagents, and methods disclosed herein. For example target ssDNA detection by Cas13a can be employed in a DETECTR assay disclosed herein.

Described herein are reagents comprising a single stranded detector nucleic acid comprising a detection moiety, wherein the detector nucleic acid is capable of being cleaved by the activated nuclease, thereby generating a first detectable signal. As used herein, a detector nucleic acid is used interchangeably with reporter or reporter molecule. In some cases, the detector nucleic acid is a single-stranded nucleic acid comprising deoxyribonucleotides. In other cases, the detector nucleic acid is a single-stranded nucleic acid comprising ribonucleotides. The detector nucleic acid can be a single-stranded nucleic acid comprising at least one deoxyribonucleotide and at least one ribonucleotide. In some cases, the detector nucleic acid is a single-stranded nucleic acid comprising at least one ribonucleotide residue at an internal position that functions as a cleavage site. In some cases, the detector nucleic acid comprises at least 2, 3, 4, 5, 6, 7, 8, 9, or 10 ribonucleotide residues at an internal position. Sometimes the ribonucleotide residues are continuous. Alternatively, the ribonucleotide residues are interspersed in between non-ribonucleotide residues. In some cases, the detector nucleic acid has only ribonucleotide residues. In some cases, the detector nucleic acid has only deoxyribonucleotide residues. In some cases, the detector nucleic acid comprises nucleotides resistant to cleavage by the programmable nuclease described herein. In some cases, the detector nucleic acid comprises synthetic nucleotides. In some cases, the detector nucleic acid comprises at least one ribonucleotide residue and at least one non-ribonucleotide residue. In some cases, detector nucleic acid is 5-20, 5-15, 5-10, 7-20, 7-15, or 7-10 nucleotides in length. In some cases, the detector nucleic acid comprises at least one uracil ribonucleotide. In some cases, the detector nucleic acid comprises at least two uracil ribonucleotides. Sometimes the detector nucleic acid has only uracil ribonucleotides. In some cases, the detector nucleic acid comprises at least one adenine ribonucleotide. In some cases, the detector nucleic acid comprises at least two adenine ribonucleotide. In some cases, the detector nucleic acid has only adenine ribonucleotides. In some cases, the detector nucleic acid comprises at least one cytosine ribonucleotide. In some cases, the detector nucleic acid comprises at least two cytosine ribonucleotide. In some cases, the detector nucleic acid comprises at least one guanine ribonucleotide. In some cases, the detector nucleic acid comprises at least two guanine ribonucleotide. A detector nucleic acid can comprise only unmodified ribonucleotides, only unmodified deoxyribonucleotides, or a combination thereof. In some cases, the detector nucleic acid is from 5 to 12 nucleotides in length. In some cases, the detector nucleic acid is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the detector nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising Cas13, a detector nucleic acid can be 5, 8, or 10 nucleotides in length. For cleavage by a programmable nuclease comprising Cas12, a detector nucleic acid can be 10 nucleotides in length.

The single stranded detector nucleic acid comprises a detection moiety capable of generating a first detectable signal. Sometimes the detector nucleic acid comprises a protein capable of generating a signal. A signal can be a calorimetric, potentiometric, amperometric, optical (e.g., fluorescent, colorometric, etc.), or piezo-electric signal. In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the detector nucleic acid. Sometimes the detection moiety is at the 3′ terminus of the detector nucleic acid. In some cases, the detection moiety is at the 5′ terminus of the detector nucleic acid. In some cases, the quenching moiety is at the 3′ terminus of the detector nucleic acid. In some cases, the single-stranded detector nucleic acid is at least one population of the single-stranded nucleic acid capable of generating a first detectable signal. In some cases, the single-stranded detector nucleic acid is a population of the single stranded nucleic acid capable of generating a first detectable signal. Optionally, there is more than one population of single-stranded detector nucleic acid. In some cases, there are 2, 3, 4, 5, 6, 7, 8, 9, 10, 11, 12, 13, 14, 15, 20, 30, 40, 50, or greater than 50, or any number spanned by the range of this list of different populations of single-stranded detector nucleic acids capable of generating a detectable signal.

TABLE 5
Exemplary Single Stranded Detector Nucleic Acid

| 5′ Detection Moiety* | Sequence (SEQ ID NO:) | 3′ Quencher* |
|---|---|---|
| /56-FAM/ | rUrUrUrUrU (SEQ ID NO: 1) | /3IABkFQ/ |
| /5IRD700/ | rUrUrUrUrU (SEQ ID NO: 1) | /3IRQC1N/ |
| /5TYE665/ | rUrUrUrUrU (SEQ ID NO: 1) | /3IAbRQSp/ |
| /5Alex594N/ | rUrUrUrUrU (SEQ ID NO: 1) | /3IAbRQSp/ |
| /5ATTO633N/ | rUrUrUrUrU (SEQ ID NO: 1) | /3IAbRQSp/ |
| /56-FAM/ | rUrUrUrUrUrUrUrU (SEQ ID NO: 2) | /3IABkFQ/ |
| /5IRD700/ | rUrUrUrUrUrUrUrU (SEQ ID NO: 2) | /3IRQC1N/ |
| /5TYE665/ | rUrUrUrUrUrUrUrU (SEQ ID NO: 2) | /3IAbRQSp/ |
| /5Alex594N/ | rUrUrUrUrUrUrUrU (SEQ ID NO: 2) | /3IAbRQSp/ |
| /5ATTO633N/ | rUrUrUrUrUrUrUrU (SEQ ID NO: 2) | /3IAbRQSp/ |
| /56-FAM/ | rUrUrUrUrUrUrUrUrUrU (SEQ ID NO: 3) | /3IABkFQ/ |
| /5IRD700/ | rUrUrUrUrUrUrUrUrUrU (SEQ ID NO: 3) | /3IRQC1N/ |
| /5TYE665/ | rUrUrUrUrUrUrUrUrUrU (SEQ ID NO: 3) | /3IAbRQSp/ |
| /5Alex594N/ | rUrUrUrUrUrUrUrUrUrU (SEQ ID NO: 3) | /3IAbRQSp/ |
| /5ATTO633N/ | rUrUrUrUrUrUrUrUrUrU (SEQ ID NO: 3) | /3IAbRQSp/ |
| /56-FAM/ | TTTTrUrUTTTT (SEQ ID NO: 4) | /3IABkFQ/ |
| /5IRD700/ | TTTTrUrUTTTT (SEQ ID NO: 4) | /3IRQC1N/ |
| /5TYE665/ | TTTTrUrUTTTT (SEQ ID NO: 4) | /3IAbRQSp/ |
| /5Alex594N/ | TTTTrUrUTTTT (SEQ ID NO: 4) | /3IAbRQSp/ |
| /5ATTO633N/ | TTTTrUrUTTTT (SEQ ID NO: 4) | /3IAbRQSp/ |
| /56-FAM/ | TTrUrUTT (SEQ ID NO: 5) | /3IABkFQ/ |
| /5IRD700/ | TTrUrUTT (SEQ ID NO: 5) | /3IRQC1N/ |
| /5TYE665/ | TTrUrUTT (SEQ ID NO: 5) | /3IAbRQSp/ |
| /5Alex594N/ | TTrUrUTT (SEQ ID NO: 5) | /3IAbRQSp/ |
| /5ATTO633N/ | TTrUrUTT (SEQ ID NO: 5) | /3IAbRQSp/ |
| /56-FAM/ | TArArUGC (SEQ ID NO: 6) | /3IABkFQ/ |
| /5IRD700/ | TArArUGC (SEQ ID NO: 6) | /3IRQC1N/ |
| /5TYE665/ | TArArUGC (SEQ ID NO: 6) | /3IAbRQSp/ |
| /5Alex594N/ | TArArUGC (SEQ ID NO: 6) | /3IAbRQSp/ |
| /5ATTO633N/ | TArArUGC (SEQ ID NO: 6) | /3IAbRQSp/ |
| /56-FAM/ | TArUrGGC (SEQ ID NO: 7) | /3IABkFQ/ |
| /5IRD700/ | TArUrGGC (SEQ ID NO: 7) | /3IRQC1N/ |
| /5TYE665/ | TArUrGGC (SEQ ID NO: 7) | /3IAbRQSp/ |
| /5Alex594N/ | TArUrGGC (SEQ ID NO: 7) | /3IAbRQSp/ |
| /5ATTO633N/ | TArUrGGC (SEQ ID NO: 7) | /3IAbRQSp/ |
| /56-FAM/ | rUrUrUrUrU (SEQ ID NO: 8) | /3IABkFQ/ |
| /5IRD700/ | rUrUrUrUrU (SEQ ID NO: 8) | /3IRQC1N/ |
| /5TYE665/ | rUrUrUrUrU (SEQ ID NO: 8) | /3IAbRQSp/ |
| /5Alex594N/ | rUrUrUrUrU (SEQ ID NO: 8) | /3IAbRQSp/ |
| /5ATTO633N/ | rUrUrUrUrU (SEQ ID NO: 8) | /3IAbRQSp/ |
| /56-FAM/ | TTATTATT (SEQ ID NO: 9) | /3IABkFQ/ |
| /56-FAM/ | TTATTATT (SEQ ID NO: 9) | /3IABkFQ/ |
| /5IRD700/ | TTATTATT (SEQ ID NO: 9) | /3IRQC1N/ |
| /5TYE665/ | TTATTATT (SEQ ID NO: 9) | /3IAbRQSp/ |
| /5Alex594N/ | TTATTATT (SEQ ID NO: 9) | /3IAbRQSp/ |
| /5ATTO633N/ | TTATTATT (SEQ ID NO: 9) | /3IAbRQSp/ |
| /56-FAM/ | TTTTTT (SEQ ID NO: 10) | /3IABkFQ/ |
| /56-FAM/ | TTTTTTTT (SEQ ID NO: 11) | /3IABkFQ/ |
| /56-FAM/ | TTTTTTTTTT (SEQ ID NO: 12) | /3IABkFQ/ |
| /56-FAM/ | TTTTTTTTTTTT (SEQ ID NO: 13) | /3IABkFQ/ |
| /56-FAM/ | TTTTTTTTTTTTTT (SEQ ID NO: 14) | /3IABkFQ/ |
| /56-FAM/ | AAAAAA (SEQ ID NO: 15) | /3IABkFQ/ |
| /56-FAM/ | CCCCCC (SEQ ID NO: 16) | /3IABkFQ/ |
| /56-FAM/ | GGGGGG (SEQ ID NO: 17) | /3IABkFQ/ |
| /56-FAM/ | TTATTATT (SEQ ID NO: 9) | /3IABkFQ/ |

/56-FAM/: 5′ 6-Fluorescein (Integrated DNA Technologies)
/3IABkFQ/: 3′ Iowa Black FQ (Integrated DNA Technologies)
/5IRD700/: 5′ IRDye 700 (Integrated DNA Technologies)
/5TYE665/: 5′ TYE 665 (Integrated DNA Technologies)
/5Alex594N/: 5′ Alexa Fluor 594 (NHS Ester) (Integrated DNA Technologies)
/5ATTO633N/: 5′ ATTO TM 633 (NHS Ester) (Integrated DNA Technologies)
/3IRQC1N/: 3′ IRDye QC-1 Quencher (Li-Cor)
/3IAbRQSp/: 3′ Iowa Black RQ (Integrated DNA Technologies)
rU: uracil ribonucleotide
rG: guanine ribonucleotide
*This Table refers to the detection moiety and quencher moiety as their tradenames and their source is identified. However, alternatives, generics, or non-tradename moieties with similar function from other sources can also be used.

A detection moiety can be an infrared fluorophore. A detection moiety can be a fluorophore that emits fluorescence in the range of from 500 nm and 720 nm. A detection moiety can be a fluorophore that emits fluorescence in the range of from 500 nm and 720 nm. In some cases, the detection moiety emits fluorescence at a wavelength of 700 nm or higher. In other cases, the detection moiety emits fluorescence at about 660 nm or about 670 nm. In some cases, the detection moiety emits fluorescence at in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 6890 to 690, 690 to 700, 700 to 710, 710 to 720, or 720 to 730 nm. A detection moiety can be a fluorophore that emits a fluorescence in the same range as 6-Fluorescein, IRDye 700, TYE 665, Alex Fluor, or ATTO TM 633 (NHS Ester). A detection moiety can be fluorescein amidite, 6-Fluorescein, IRDye 700, TYE 665, Alex Fluor 594, or ATTO TM 633 (NHS Ester). A detection moiety can be a fluorophore that emits a fluorescence in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A detection moiety can be fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Any of the detection moieties described herein can be from any commercially available source, can be an alternative with a similar function, a generic, or a non-tradename of the detection moieties listed.

A detection moiety can be chosen for use based on the type of sample to be tested. For example, a detection moiety that is an infrared fluorophore is used with a urine sample. As another example, SEQ ID NO: 1 with a fluorophore that emits around 520 nm is used for testing in non-urine samples, and SEQ ID NO: 8 with a fluorophore that emits a fluorescence around 700 nm is used for testing in urine samples.

A quenching moiety can be chosen based on its ability to quench the detection moiety. A quenching moiety can be a non-fluorescent fluorescence quencher. A quenching moiety can quench a detection moiety that emits fluorescence in the range of from 500 nm and 720 nm. A quenching moiety can quench a detection moiety that emits fluorescence in the range of from 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety emits fluorescence at in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 6890 to 690, 690 to 700, 700 to 710, 710 to 720, or 720 to 730 nm. A quenching moiety can quench fluorescein amidite, 6-Fluorescein, IRDye 700, TYE 665, Alex Fluor 594, or ATTO TM 633 (NHS Ester). A quenching moiety can be Iowa Black RQ, Iowa Black FQ or IRDye QC-1 Quencher. A quenching moiety can quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety can be Iowa Black RQ (Integrated DNA Technologies), Iowa Black FQ (Integrated DNA Technologies) or IRDye QC-1 Quencher (LiCor). Any of the quenching moieties described herein can be from any commercially available source, can be an alternative with a similar function, a generic, or a non-tradename of the quenching moieties listed.

The generation of the detectable signal from the release of the detection moiety indicates that cleavage by the programmable nuclease has occurred and that the sample contains the target nucleic acid. In some cases, the detection moiety comprises a fluorescent dye. Sometimes the detection moiety comprises a fluorescence resonance energy transfer (FRET) pair. In some cases, the detection moiety comprises an infrared (IR) dye. In some cases, the detection moiety comprises an ultraviolet (UV) dye. Alternatively or in combination, the detection moiety comprises a polypeptide. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

A detection moiety can be any moiety capable of generating a calorimetric, potentiometric, amperometric, optical (e.g., fluorescent, colorimetric, etc.), or piezo-electric signal. A detector nucleic acid, sometimes, is protein-nucleic acid that is capable of generating a calorimetric, potentiometric, amperometric, optical (e.g., fluorescent, colorimetric, etc.), or piezo-electric signal upon cleavage of the nucleic acid. A protein-nucleic acid may comprise a nucleic acid component and a protein or peptide component. In some embodiments, a protein-nucleic acid may comprise a nucleic acid fused to a protein or peptide. Often a calorimetric signal is heat produced after cleavage of the detector nucleic acids. Sometimes, a calorimetric signal is heat absorbed after cleavage of the detector nucleic acids. A potentiometric signal, for example, is electrical potential produced after cleavage of the detector nucleic acids. An amperometric signal can be movement of electrons produced after the cleavage of detector nucleic acid. Often, the signal is an optical signal, such as a colorimetric signal or a fluorescence signal. An optical signal is, for example, a light output produced after the cleavage of the detector nucleic acids. Sometimes, an optical signal is a change in light absorbance between before and after the cleavage of detector nucleic acids. Often, a piezo-electric signal is a change in mass between before and after the cleavage of the detector nucleic acid.

Often, the protein-nucleic acid is an enzyme-nucleic acid. The enzyme may be sterically hindered when present as in the enzyme-nucleic acid, but then functional upon cleavage from the nucleic acid. Often, the enzyme is an enzyme that produces a reaction with a substrate. An enzyme can be invertase. Often, the substrate of invertase is sucrose and DNS reagent.

Sometimes the protein-nucleic acid is a substrate-nucleic acid. Often the substrate is a substrate that produces a reaction with an enzyme.

A protein-nucleic acid may be attached to a solid support. The solid support, for example, is a surface. A surface can be an electrode. Sometimes the solid support is a bead. Often the bead is a magnetic bead. Upon cleavage, the protein is liberated from the solid and interacts with other mixtures. For example, the protein is an enzyme, and upon cleavage of the nucleic acid of the enzyme-nucleic acid, the enzyme flows through a chamber into a mixture comprising the substrate. When the enzyme meets the enzyme substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

In some embodiments, the reporter comprises a nucleic acid conjugated to an affinity molecule and the affinity molecule conjugated to the fluorophore (e.g., nucleic acid—affinity molecule—fluorophore) or the nucleic acid conjugated to the fluorophore and the fluorophore conjugated to the affinity molecule (e.g., nucleic acid—fluorophore—affinity molecule). In some embodiments, a linker conjugates the nucleic acid to the affinity molecule. In some embodiments, a linker conjugates the affinity molecule to the fluorophore. In some embodiments, a linker conjugates the nucleic acid to the fluorophore. A linker can be any suitable linker known in the art. In some embodiments, the nucleic acid of the reporter can be directly conjugated to the affinity molecule and the affinity molecule can be directly conjugated to the fluorophore or the nucleic acid can be directly conjugated to the fluorophore and the fluorophore can be directly conjugated to the affinity molecule. In this context, “directly conjugated” indicated that no intervening molecules, polypeptides, proteins, or other moieties are present between the two moieties directly conjugated to each other. For example, if a reporter comprises a nucleic acid directly conjugated to an affinity molecule and an affinity molecule directly conjugated to a fluorophore—no intervening moiety is present between the nucleic acid and the affinity molecule and no intervening moiety is present between the affinity molecule and the fluorophore. The affinity molecule can be biotin, avidin, streptavidin, or any similar molecule.

In some cases, the reporter comprises a substrate-nucleic acid. The substrate may be sequestered from its cognate enzyme when present as in the substrate-nucleic acid, but then is released from the nucleic acid upon cleavage, wherein the released substrate can contact the cognate enzyme to produce a detectable signal. Often, the substrate is sucrose and the cognate enzyme is invertase, and a DNS reagent can be used to monitor invertase activity.

A major advantage of the devices and methods disclosed herein is the design of excess reporters to total nucleic acids in an unamplified or an amplified sample, not including the nucleic acid of the reporter. Total nucleic acids can include the target nucleic acids and non-target nucleic acids, not including the nucleic acid of the reporter. The non-target nucleic acids can be from the original sample, either lysed or unlysed. The non-target nucleic acids can also be byproducts of amplification. Thus, the non-target nucleic acids can include both non-target nucleic acids from the original sample, lysed or unlysed, and from an amplified sample. The presence of a large amount of non-target nucleic acids, an activated programmable nuclease may be inhibited in its ability to bind and cleave the reporter sequences. This is because the activated programmable nucleases collaterally cleaves any nucleic acids. If total nucleic acids are in present in large amounts, they may outcompete reporters for the programmable nucleases. The devices and methods disclosed herein are designed to have an excess of reporter to total nucleic acids, such that the detectable signals from cleavage reactions (e.g., DETECTR reactions) are particularly superior. In some embodiments, the reporter can be present in at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, from 1.5 fold to 100 fold, from 2 fold to 10 fold, from 10 fold to 20 fold, from 20 fold to 30 fold, from 30 fold to 40 fold, from 40 fold to 50 fold, from 50 fold to 60 fold, from 60 fold to 70 fold, from 70 fold to 80 fold, from 80 fold to 90 fold, from 90 fold to 100 fold, from 1.5 fold to 10 fold, from 1.5 fold to 20 fold, from 10 fold to 40 fold, from 20 fold to 60 fold, or from 10 fold to 80 fold excess of total nucleic acids.

A second significant advantage of the devices and methods disclosed herein is the design of an excess volume comprising the guide nucleic acid, the programmable nuclease, and the reporter, which contacts a smaller volume comprising the sample with the target nucleic acid of interest. The smaller volume comprising the sample can be unlysed sample, lysed sample, or lysed sample which has undergone any combination of reverse transcription, amplification, and in vitro transcription. The presence of various reagents in a crude, non-lysed sample, a lysed sample, or a lysed and amplified sample, such as buffer, magnesium sulfate, salts, the pH, a reducing agent, primers, dNTPs, NTPs, cellular lysates, non-target nucleic acids, primers, or other components, can inhibit the ability of the programmable nuclease to find and cleave the nucleic acid of the reporter. This may be due to nucleic acids that are not the reporter, which outcompete the nucleic acid of the reporter, for the programmable nuclease. Alternatively, various reagents in the sample may simply inhibit the activity of the programmable nuclease. Thus, the devices and methods provided herein for contacting an excess volume comprising the guide nucleic acid, the programmable nuclease, and the reporter to a smaller volume comprising the sample with the target nucleic acid of interest provides for superior detection of the target nucleic acid by ensuring that the programmable nuclease is able to find and cleaves the nucleic acid of the reporter. In some embodiments, the volume comprising the guide nucleic acid, the programmable nuclease, and the reporter (can be referred to as “a second volume”) is 4-fold greater than a volume comprising the sample (can be referred to as “a first volume”). In some embodiments, the volume comprising the guide nucleic acid, the programmable nuclease, and the reporter (can be referred to as “a second volume”) is at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, from 1.5 fold to 100 fold, from 2 fold to 10 fold, from 10 fold to 20 fold, from 20 fold to 30 fold, from 30 fold to 40 fold, from 40 fold to 50 fold, from 50 fold to 60 fold, from 60 fold to 70 fold, from 70 fold to 80 fold, from 80 fold to 90 fold, from 90 fold to 100 fold, from 1.5 fold to 10 fold, from 1.5 fold to 20 fold, from 10 fold to 40 fold, from 20 fold to 60 fold, or from 10 fold to 80 fold greater than a volume comprising the sample (can be referred to as “a first volume”). In some embodiments, the volume comprising the sample is at least 0.5 ul, at least 1 ul, at least at least 1 uL, at least 2 uL, at least 3 uL, at least 4 uL, at least 5 uL, at least 6 uL, at least 7 uL, at least 8 uL, at least 9 uL, at least 10 uL, at least 11 uL, at least 12 uL, at least 13 uL, at least 14 uL, at least 15 uL, at least 16 uL, at least 17 uL, at least 18 uL, at least 19 uL, at least 20 uL, at least 25 uL, at least 30 uL, at least 35 uL, at least 40 uL, at least 45 uL, at least 50 uL, at least 55 uL, at least 60 uL, at least 65 uL, at least 70 uL, at least 75 uL, at least 80 uL, at least 85 uL, at least 90 uL, at least 95 uL, at least 100 uL, from 0.5 uL to 5 ul uL, from 5 uL to 10 uL, from 10 uL to 15 uL, from 15 uL to 20 uL, from 20 uL to 25 uL, from 25 uL to 30 uL, from 30 uL to 35 uL, from 35 uL to 40 uL, from 40 uL to 45 uL, from 45 uL to 50 uL, from 10 uL to 20 uL, from 5 uL to 20 uL, from 1 uL to 40 uL, from 2 uL to 10 uL, or from 1 uL to 10 uL. In some embodiments, the volume comprising the programmable nuclease, the guide nucleic acid, and the reporter is at least 10 uL, at least 11 uL, at least 12 uL, at least 13 uL, at least 14 uL, at least 15 uL, at least 16 uL, at least 17 uL, at least 18 uL, at least 19 uL, at least 20 uL, at least 21 uL, at least 22 uL, at least 23 uL, at least 24 uL, at least 25 uL, at least 26 uL, at least 27 uL, at least 28 uL, at least 29 uL, at least 30 uL, at least 40 uL, at least 50 uL, at least 60 uL, at least 70 uL, at least 80 uL, at least 90 uL, at least 100 uL, at least 150 uL, at least 200 uL, at least 250 uL, at least 300 uL, at least 350 uL, at least 400 uL, at least 450 uL, at least 500 uL, from 10 uL to 15 ul uL, from 15 uL to 20 uL, from 20 uL to 25 uL, from 25 uL to 30 uL, from 30 uL to 35 uL, from 35 uL to 40 uL, from 40 uL to 45 uL, from 45 uL to 50 uL, from 50 uL to 55 uL, from 55 uL to 60 uL, from 60 uL to 65 uL, from 65 uL to 70 uL, from 70 uL to 75 uL, from 75 uL to 80 uL, from 80 uL to 85 uL, from 85 uL to 90 uL, from 90 uL to 95 uL, from 95 uL to 100 uL, from 100 uL to 150 uL, from 150 uL to 200 uL, from 200 uL to 250 uL, from 250 uL to 300 uL, from 300 uL to 350 uL, from 350 uL to 400 uL, from 400 uL to 450 uL, from 450 uL to 500 uL, from 10 uL to 20 uL, from 10 uL to 30 uL, from 25 uL to 35 uL, from 10 uL to 40 uL, from 20 uL to 50 uL, from 18 uL to 28 uL, or from 17 uL to 22 uL.

A reporter may be a hybrid nucleic acid reporter. A hybrid nucleic acid reporter comprises a nucleic acid with at least one deoxyribonucleotide and at least one ribonucleotide. In some embodiments, the nucleic acid of the hybrid nucleic acid reporter can be of any length and can have any mixture of DNAs and RNAs. For example, in some cases, longer stretches of DNA can be interrupted by a few ribonucleotides. Alternatively, longer stretches of RNA can be interrupted by a few deoxyribonucleotides. Alternatively, every other base in the nucleic acid may alternate between ribonucleotides and deoxyribonucleotides. A major advantage of the hybrid nucleic acid reporter is increased stability as compared to a pure RNA nucleic acid reporter. For example, a hybrid nucleic acid reporter can be more stable in solution, lyophilized, or vitrified as compared to a pure DNA or pure RNA reporter.

The reporter can be lyophilized or vitrified. The reporter can be suspended in solution or immobilized on a surface. For example, the reporter can be immobilized on the surface of a chamber in a device as disclosed herein. In some cases, the reporter is immobilized on beads, such as magnetic beads, in a chamber of a device as disclosed herein where they are held in position by a magnet placed below the chamber.

Additionally, target nucleic acid can be amplified before binding to the crRNA of the CRISPR enzyme. This amplification can be PCR amplification or isothermal amplification. This nucleic acid amplification of the sample can improve at least one of sensitivity, specificity, or accuracy of the detection the target RNA. The reagents for nucleic acid amplification can comprise a recombinase, an oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C. The nucleic acid amplification reaction can be performed at a temperature of at least 20° C., 25° C., 30° C., 35° C., 37° C., 40° C., or 45° C.

Disclosed herein are methods of assaying for a target nucleic acid as described herein wherein a signal is detected. For example, a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

A programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby nucleic acids by the activated programmable nuclease, such as trans cleavage of detector nucleic acids with a detection moiety. Once the detector nucleic acid is cleaved by the activated programmable nuclease, the detection moiety can be released from the detector nucleic acid and can generate a signal. The signal can be immobilized on a support medium for detection. The signal can be visualized to assess whether a target nucleic acid comprises a modification.

Often, the signal is a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorometric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of the detection moiety to the capture molecule in the detection region, where the first detection signal indicates that the sample contained the target nucleic acid. Sometimes the system is capable of detecting more than one type of target nucleic acid, wherein the system comprises more than one type of guide nucleic acid and more than one type of detector nucleic acid. In some cases, the detectable signal is generated directly by the cleavage event. Alternatively or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium. In some cases, the second detectable signal is generated in a spatially distinct location than the first generated signal.

In some cases, the threshold of detection, for a subject method of detecting a single stranded target nucleic acid in a sample, is less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 aM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, fom 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases, the minimum concentration at which a single stranded target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a single stranded target nucleic acid can be detected in a sample is in a range of from 1 aM to 100 pM. In some cases, the minimum concentration at which a single stranded target nucleic acid can be detected in a sample is in a range of from 1 fM to 100 pM. In some cases, the minimum concentration at which a single stranded target nucleic acid can be detected in a sample is in a range of from 10 fM to 100 pM. In some cases, the minimum concentration at which a single stranded target nucleic acid can be detected in a sample is in a range of from 800 fM to 100 pM. In some cases, the minimum concentration at which a single stranded target nucleic acid can be detected in a sample is in a range of from 1 pM to 10 pM. In some cases, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some cases, the devices, systems, fluidic devices, kits, and methods described herein detect a target single-stranded nucleic acid in a sample where the sample is contacted with the reagents for a predetermined length of time sufficient for the trans cleavage to occur or cleavage reaction to reach completion. In some cases, the devices, systems, fluidic devices, kits, and methods described herein detect a target single-stranded nucleic acid in a sample where the sample is contacted with the reagents for no greater than 60 minutes. Sometimes the sample is contacted with the reagents for no greater than 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, or 1 minute. Sometimes the sample is contacted with the reagents for at least 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, or 5 minutes. In some cases, the devices, systems, fluidic devices, kits, and methods described herein can detect a target nucleic acid in a sample in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes.

When a guide nucleic acid binds to a target nucleic acid, the programmable nuclease's trans cleavage activity can be initiated, and detector nucleic acids can be cleaved, resulting in the detection of fluorescence. Some methods as described herein can a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The cleaving of the detector nucleic acid using the programmable nuclease may cleave with an efficiency of 50% as measured by a change in a signal that is calorimetric, potentiometric, amperometric, optical (e.g., fluorescent, colorimetric, etc.), or piezo-electric, as non-limiting examples. Some methods as described herein can be a method of detecting a target nucleic acid in a sample comprising contacting the sample comprising the target nucleic acid with a guide nucleic acid targeting a target nucleic acid segment, a programmable nuclease capable of being activated when complexed with the guide nucleic acid and the target nucleic acid segment, a single stranded detector nucleic acid comprising a detection moiety, wherein the detector nucleic acid is capable of being cleaved by the activated programmable nuclease, thereby generating a first detectable signal, cleaving the single stranded detector nucleic acid using the programmable nuclease that cleaves as measured by a change in color, and measuring the first detectable signal on the support medium. The cleaving of the single stranded detector nucleic acid using the programmable nuclease may cleave with an efficiency of 50% as measured by a change in color. In some cases, the cleavage efficiency is at least 40%, 50%, 60%, 70%, 80%, 90%, or 95% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with a guide nucleic acid targeting a target nucleic acid segment, a programmable nuclease capable of being activated when complexed with the guide nucleic acid and the target nucleic acid segment, and a single stranded detector nucleic acid comprising a detection moiety, wherein the detector nucleic acid is capable of being cleaved by the activated nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample.

In some cases, the devices, systems, fluidic devices, kits, and methods described herein detect a target single-stranded nucleic acid with a programmable nuclease and a single-stranded detector nucleic acid in a sample where the sample is contacted with the reagents for a predetermined length of time sufficient for trans cleavage of the single stranded detector nucleic acid. For example, a programmable nuclease is LbuCas13a that detects a target nucleic acid and a single stranded detector nucleic acid comprises two adjacent uracil nucleotides with a green detectable moiety that is detected upon cleavage. As another example, a programmable nuclease is LbaCas13a that detects a target nucleic acid and a single-stranded detector nucleic acid comprises two adjacent adenine nucleotides with a red detectable moiety that is detected upon cleavage.

In some cases, the devices, systems, fluidic devices, kits, and methods described herein detect different two target single-stranded nucleic acids with two different programmable nucleases and two different single-stranded detector nucleic acids in a sample where the sample is contacted with the reagents for a predetermined length of time sufficient for trans cleavage of the at least two single-stranded detector nucleic acids. For example, a first programmable nuclease is LbuCas13a, which is activated by a first single-stranded target nucleic acid and upon activation, cleaves a first single-stranded detector nucleic acid comprising two adjacent uracil nucleotides with a green detectable moiety that is detected upon cleavage, and a second programmable nuclease is LbaCas13a, which is activated by a second single-stranded target nucleic acid and upon activation, cleaves a second single-stranded detector nucleic acid comprising two adjacent adenine nucleotides with a red detectable moiety that is detected upon cleavage. In some cases, the activation of both programmable nucleases to cleave their respective single-stranded nucleic acids, for example LbuCas13a that cleaves a first single-stranded detector nucleic acid comprising two adjacent uracil nucleotides with a green detectable moiety that is detected upon cleavage and LbaCas13a that cleaves a second single-stranded detector nucleic acid comprises two adjacent adenine nucleotides with a red detectable moiety that is detected upon cleavage, the subsequence detection of a yellow signal indicates that the first single-stranded target nucleic acid and the second single-stranded target nucleic are present in the sample.

Alternatively, the devices, systems, fluidic devices, kits, and methods described herein can comprise a first programmable nuclease that detects the presence of a first single-stranded target nucleic acid in a sample and a second programmable nuclease that is used as a control. For example, a first programmable nuclease is Lbu13a, which cleaves a first single-stranded detector nucleic acid comprising two adjacent uracil nucleotides with a green detectable moiety that is detected upon cleavage and which is activated by a first single-stranded target nucleic acid if it is present in the sample, and a second programmable nuclease is Lba13a, which cleaves a second single-stranded detector nucleic acid comprising two adjacent adenine nucleotides with a red detectable moiety that is detected upon cleavage and which is activated by a second single-stranded target nucleic acid that is not found (and would not be expected to ever be found) in the sample and serves as a control. In this case, the detection of a red signal or a yellow signal indicates there is a problem with the test (e.g., the sample contains a high level of other RNAses that are cleaving the single-stranded detector nucleic acids in the absence of activation of the second programmable nuclease), but the detection of a green signal indicates the test is working correctly and the first target single-stranded nucleic acid of the first programmable nuclease is present in the sample.

As additional examples, the devices, systems, fluidic devices, kits, and methods described herein detect different two target single-stranded nucleic acids with two different programmable nucleases and two different single stranded detector nucleic acids in a sample where the sample is contacted with the reagents for a predetermined length of time sufficient for trans cleavage of the at least two single stranded detector nucleic acid. For example, a first programmable nuclease is a Cas13a protein, which cleaves a first single-stranded detector nucleic that is detected upon cleavage and which is activated by a first single-stranded target nucleic acid from a sepsis RNA biomarker if it is present in the sample, and a second programmable nuclease is a Cas14 protein, which cleaves a second single-stranded detector nucleic acid that is detected upon cleavage and which is activated by a second single-stranded target nucleic acid from in influenza virus.

The reagents described herein can also include buffers, which are compatible with the devices, systems, fluidic devices, kits, and methods disclosed herein. These buffers are compatible with the other reagents, samples, and support mediums as described herein for detection of an ailment, such as a disease, including those caused by viruses such as influenza. The methods described herein can also include the use of buffers, which are compatible with the methods disclosed herein. For example, a buffer comprises 20 mM HEPES pH 6.8, 50 mM KCl, 5 mM MgCl₂, and 5% glycerol. In some instances the buffer comprises from 0 to 100, 0 to 75, 0 to 50, 0 to 25, 0 to 20, 0 to 10, 0 to 5, 5 to 10,5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM HEPES pH 6.8. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In other instances the buffer comprises 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl₂. The buffer can comprise 0 to 25, 0 to 20, 0 to 10, 0 to 5, 5 to 10, 5 to 15, 5 to 20, 5 to 25, 5 to 30% glycerol.

As another example, a buffer comprises 100 mM Imidazole pH 7.5; 250 mM KCl, 25 mM MgCl₂, 50 ug/mL BSA, 0.05% Igepal Ca-630, and 25% Glycerol. In some instances the buffer comprises 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM Imidazole pH 7.5. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In other instances the buffer comprises 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl₂. The buffer, in some instances, comprises 0 to 100, 0 to 75, 0 to 50, 0 to 25, 0 to 20, 0 to 10, 0 to 5, 5 to 50, 5 to 75, 5 to 100, 10 to 20, 10 to 50, 10 to 75, 10 to 100, 25 to 50, 25 to 75 25 to 100, 50 to 75, or 50 to 100 ug/mL BSA. In some instances, the buffer comprises 0 to 1, 0 to 0.5, 0 to 0.25, 0 to 0.01, 0 to 0.05, 0 to 0.025, 0 to 0.01, 0.01 to 0.025, 0.01 to 0.05, 0.01 to 0.1, 0.01 to 0.25, 0.01, to 0.5, 0.01 to 1, 0.025 to 0.05, 0.025 to 0.1, 0.025, to 0.5, 0.025 to 1, 0.05 to 0.1, 0.05 to 0.25, 0.05 to 0.5, 0.05 to 0.75, 0.05 to 1, 0.1 to 0.25, 0.1 to 0.5, or 0.1 to 1% Igepal Ca-630. The buffer can comprise 0 to 25, 0 to 20, 0 to 10, 0 to 5, 5 to 10, 5 to 15, 5 to 20, 5 to 25, 5 to 30% glycerol.

A buffer of the present disclosure may comprise a viral lysis buffer. A viral lysis buffer may lyse a coronavirus capsid in a viral sample (e.g., a sample collected from an individual suspected of having a coronavirus infection), releasing a viral genome. The viral lysis buffer may be compatible with amplification (e.g., RT-LAMP amplification) of a target region of the viral genome. The viral lysis buffer may be compatible with detection (e.g., a DETECTR reaction disclosed herein). A viral lysis buffer that is functional to lyse a virus and is compatible with amplification, detection, or both may be a dual lysis buffer. A viral lysis buffer that is functional to lyse a virus and is compatible with amplification may be a dual lysis/amplification buffer. A viral lysis buffer that is functional to lyse a virus and is compatible with detection may be a dual lysis/detection buffer. A sample may be prepared in a one-step sample preparation method comprising suspending the sample in a viral lysis buffer compatible with amplification, detection (e.g., a DETECTR reaction), or both. A viral lysis buffer compatible with amplification (e.g., RT-LAMP amplification), detection (e.g., DETECTR), or both, may comprise a buffer (e.g., Tris-HCl, phosphate, or HEPES), a reducing agent (e.g., N-Acetyl Cysteine (NAC), Dithiothreitol (DTT), β-mercaptoethanol (BME), or tris(2-carboxyethyl)phosphine (TCEP)), a chelating agent (e.g., EDTA or EGTA), a detergent (e.g., deoxycholate, NP-40 (Ipgal), Triton X-100, or Tween 20), a salt (e.g., ammonium acetate, magnesium acetate, manganese acetate, potassium acetate, sodium acetate, ammonium chloride, potassium chloride, magnesium chloride, manganese chloride, sodium chloride, ammonium sulfate, magnesium sulfate, manganese sulfate, potassium sulfate, or sodium sulfate), or a combination thereof. For example, a viral lysis buffer may comprise a buffer and a reducing agent, or a viral lysis buffer may comprise a buffer and a chelating agent. The viral lysis buffer may be formulated at a low pH. For example, the viral lysis buffer may be formulated at a pH of from about pH 4 to about pH 5. In some embodiments, the viral lysis buffer may be formulated at a pH of from about pH 4 to about pH 8.8. In some embodiments, the viral lysis buffer may be formulated at a pH of from about pH 4 to about pH 9. The viral lysis buffer may further comprise a preservative (e.g., ProClin 150). In some embodiments, the viral lysis buffer may comprise an activator of the amplification reaction. For example, the buffer may comprise primers, dNTPs, or magnesium (e.g., MgSO₄, MgCl₂ or MgOAc), or a combination thereof, to activate the amplification reaction. In some embodiments, an activator (e.g., primers, dNTPs, or magnesium) may be added to the buffer following lysis of the coronavirus to initiate the amplification reaction.

A viral lysis buffer may comprise a pH of about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 6, about 6.5, about 7, about 7.5, about 8, about 8.5, or about 9. In some embodiments, a viral lysis buffer may comprise a pH of from 3.5 to 4.5, from 4 to 5, from 4.5 to 5.5, from 3.5 to 4, from 4 to 4.5, from 4.5 to 5, from 5 to 5.5, from 5 to 6, from 6 to 7, from 7 to 8, or from 8 to 9.

A viral lysis buffer may comprise a magnesium concentration of about 0 mM, about 2 mM, about 4 mM, about 5 mM, about 6 mM, about 8 mM, about 10 mM, about 12 mM, about 13 mM, about 14 mM, about 15 mM, about 20 mM, about 25 mM, about 30 mM, about 35 mM, about 40 mM, about 45 mM, about 50 mM, about 55 mM, or about 60 mM of magnesium (e.g., MgSO₄, MgCl₂ or MgOAc). A viral lysis buffer may comprise a magnesium concentration of from 0 mM to 5 mM, from 5 mM to 10 mM, from 10 mM to 15 mM, from 15 mM to 20 mM, from 20 mM to 25 mM, from 25 mM to 30 mM, from 30 mM to 40 mM, from 40 mM to 50 mM, or from 50 mM to 60 mM of magnesium (e.g., MgSO₄, MgCl₂ or MgOAc). In some embodiments, the magnesium may be added after viral lysis to activate an amplification reaction.

A viral lysis buffer may comprise a reducing agent (e.g., NAC, DTT, BME, or TCEP) at a concentration of about 1 mM, about 2 mM, about 3 mM, about 4 mM, about 5 mM, about 6 mM, about 7 mM, about 8 mM, about 10 mM, about 12 mM, about 15 mM, about 20 mM, about 25 mM, about 30 mM, about 40 mM, about 50 mM, about 60 mM, about 7 mM, about 80 mM, about 90 mM, about 100 mM, or about 120 mM. A viral lysis buffer may comprise a reducing agent (e.g., NAC, DTT, BME, or TCEP) at a concentration of from 1 mM to 5 mM, from 5 mM to 10 mM, from 10 mM to 15 mM, from 15 mM to 20 mM, from 20 mM to 25 mM, or from 25 mM to 30 mM, from 30 mM to 40 mM, from 40 mM to 50 mM, from 50 mM to 60 mM, from 60 mM to 70 mM, from 70 mM to 80 mM, or from 80 mM to 90 mM, from 90 mM to 100 mM, or from 100 mM to 120 mM. A viral lysis buffer may comprise a chelator (e.g., EDTA or EGTA) at a concentration of about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 10 mM, about 12 mM, about 15 mM, about 20 mM, about 25 mM, or about 30 mM. A viral lysis buffer may comprise a chelator (e.g., EDTA or EGTA) at a concentration of from 0.1 mM to 0.5 mM, from 0.25 mM to 0.5 mM, from 0.4 mM to 0.6 mM, from 0.5 mM to 1 mM, from 1 mM to 5 mM, from 5 mM to 10 mM, from 10 mM to 15 mM, from 15 mM to 20 mM, from 20 mM to 25 mM, or from 25 mM to 30 mM.

A viral lysis buffer may comprise a salt (e.g., ammonium acetate ((NH₄)₂OAc), magnesium acetate (MgOAc), manganese acetate (MnOAc), potassium acetate (K₂OAc), sodium acetate (Na₂OAc), ammonium chloride (NH₄Cl), potassium chloride (KCl), magnesium chloride (MgCl₂), manganese chloride (MnCl₂), sodium chloride (NaCl), ammonium sulfate ((NH₄)₂SO₄), magnesium sulfate (MgSO₄), manganese sulfate (MnSO₄), potassium sulfate (K₂SO₄), or sodium sulfate (Na₂SO₄)) at a concentration of about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 70 mM, about 80 mM, about 90 mM, or about 100 mM. A viral lysis buffer may comprise a salt (e.g., (NH₄)₂OAc, MgOAc, MnOAc, K₂OAc, Na₂OAc, NH₄Cl, KCl, MgCl₂, MnCl₂, NaCl, (NH₄)₂SO₄, MgSO₄, MnSO₄, K₂SO₄, or Na₂SO₄) at a concentration of from 1 mM to 5 mM, from 1 mM to 10 mM, from 5 mM to 10 mM, from 10 mM to 15 mM, from 15 mM to 20 mM, from 20 mM to 25 mM, from 25 mM to 30 mM, from 30 mM to 35 mM, from 35 mM to 40 mM, from 40 mM to 45 mM, from 45 mM to 50 mM, from 50 mM to 55 mM, from 55 mM to 60 mM, from 60 mM to 70 mM, from 70 mM to 80 mM, from 80 mM to 90 mM, or from 90 mM to 100 mM.

A viral lysis buffer may comprise a detergent (e.g., deoxycholate, NP-40 (Ipgal), Triton X-100, or Tween 20) at a concentration of about 0.01%, about 0.05%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.00%, about 1.10%, about 1.20%, about 1.30%, about 1.40%, about 1.50%, about 2.00%, about 2.50%, about 3.00%, about 3.50%, about 4.00%, about 4.50%, or about 5.00%. A viral lysis buffer may comprise a detergent (e.g., deoxycholate, NP-40 (Ipgal), Triton X-100, or Tween 20) at a concentration of from 0.01% to 0.10%, from 0.05% to 0.15%, from 0.10% to 0.20%, from 0.15% to 0.25%, from 0.20% to 0.30%, from 0.25% to 0.35%, from 0.30% to 0.40%, from 0.35% to 0.45%, from 0.40% to 0.50%, from 0.45% to 0.55%, from 0.50% to 0.60%, from 0.55% to 0.65%, from 0.60% to 0.70%, from 0.65% to 0.75%, from 0.70% to 0.80%, from 0.75% to 0.85%, from 0.80% to 0.90%, from 0.85% to 0.95%, from 0.90% to 1.00%, from 0.95% to 1.10%, from 1.00% to 1.20%, from 1.10% to 1.30%, from 1.20% to 1.40%, from 1.30% to 1.50%, from 1.40% to 1.60%, from 1.50% to 2.00%, from 2.00% to 2.50%, from 2.50% to 3.00%, from 3.00% to 3.50%, from 3.50% to 4.00%, from 4.00% to 4.50%, or from 4.50% to 5.00%.

A lysis reaction may be performed at a range of temperatures. In some embodiments, a lysis reaction may be performed at about room temperature. In some embodiments, a lysis reaction may be performed at about 95° C. In some embodiments, a lysis reaction may be performed at from 1° C. to 10° C., from 4° C. to 8° C., from 10° C. to 20° C., from 15° C. to 25° C., from 15° C. to 20° C., from 18° C. to 25° C., from 18° C. to 95° C., from 20° C. to 37° C., from 25° C. to 40° C., from 35° C. to 45° C., from 40° C. to 60° C., from 50° C. to 70° C., from 60° C. to 80° C., from 70° C. to 90° C., from 80° C. to 95° C., or from 90° C. to 99° C. In some embodiments, a lysis reaction may be performed for about 5 minutes, about 15 minutes, or about 30 minutes. In some embodiments, a lysis reaction may be performed for from 2 minutes to 5 minutes, from 3 minutes to 8 minutes, from 5 minutes to 15 minutes, from 10 minutes to 20 minutes, from 15 minutes to 25 minutes, from 20 minutes to 30 minutes, from 25 minutes to 35 minutes, from 30 minutes to 40 minutes, from 35 minutes to 45 minutes, from 40 minutes to 50 minutes, from 45 minutes to 55 minutes, from 50 minutes to 60 minutes, from 55 minutes to 65 minutes, from 60 minutes to 70 minutes, from 65 minutes to 75 minutes, from 70 minutes to 80 minutes, from 75 minutes to 85 minutes, or from 80 minutes to 90 minutes.

A number of detection devices and methods are consistent with methods disclosed herein. For example, any device that can measure or detect a calorimetric, potentiometric, amperometric, optical (e.g., fluorescent, colorometric, etc.), or piezo-electric signal. Often a calorimetric signal is heat produced after cleavage of the detector nucleic acids. Sometimes, a calorimetric signal is heat absorbed after cleavage of the detector nucleic acids. A potentiometric signal, for example, is electrical potential produced after cleavage of the detector nucleic acids. An amperometric signal can be movement of electrons produced after the cleavage of detector nucleic acid. Often, the signal is an optical signal, such as a colorometric signal or a fluorescence signal. An optical signal is, for example, a light output produced after the cleavage of the detector nucleic acids. Sometimes, an optical signal is a change in light absorbance between before and after the cleavage of detector nucleic acids. Often, a piezo-electric signal is a change in mass between before and after the cleavage of the detector nucleic acid. Sometimes, the detector nucleic acid is protein-nucleic acid. Often, the protein-nucleic acid is an enzyme-nucleic acid.

The results from the detection region from a completed assay can be detected and analyzed in various ways, for example, by a glucometer. In some cases, the positive control spot and the detection spot in the detection region is visible by eye, and the results can be read by the user. In some cases, the positive control spot and the detection spot in the detection region is visualized by an imaging device or other device depending on the type of signal. Often, the imaging device is a digital camera, such a digital camera on a mobile device. The mobile device may have a software program or a mobile application that can capture an image of the support medium, identify the assay being performed, detect the detection region and the detection spot, provide image properties of the detection spot, analyze the image properties of the detection spot, and provide a result. Alternatively or in combination, the imaging device can capture fluorescence, ultraviolet (UV), infrared (IR), or visible wavelength signals. The imaging device may have an excitation source to provide the excitation energy and captures the emitted signals. In some cases, the excitation source can be a camera flash and optionally a filter. In some cases, the imaging device is used together with an imaging box that is placed over the support medium to create a dark room to improve imaging. The imaging box can be a cardboard box that the imaging device can fit into before imaging. In some instances, the imaging box has optical lenses, mirrors, filters, or other optical elements to aid in generating a more focused excitation signal or to capture a more focused emission signal. Often, the imaging box and the imaging device are small, handheld, and portable to facilitate the transport and use of the assay in remote or low resource settings.

The assay described herein can be visualized and analyzed by a mobile application (app) or a software program. Using the graphic user interface (GUI) of the app or program, an individual can take an image of the support medium, including the detection region, barcode, reference color scale, and fiduciary markers on the housing, using a camera on a mobile device. The program or app reads the barcode or identifiable label for the test type, locate the fiduciary marker to orient the sample, and read the detectable signals, compare against the reference color grid, and determine the presence or absence of the target nucleic acid, which indicates the presence of the gene, virus, or the agent responsible for the disease. The mobile application can present the results of the test to the individual. The mobile application can store the test results in the mobile application. The mobile application can communicate with a remote device and transfer the data of the test results. The test results can be viewable remotely from the remote device by another individual, including a healthcare professional. A remote user can access the results and use the information to recommend action for treatment, intervention, clean up of an environment.

### Detection of a Mutation in a Target Nucleic Acid

Disclosed herein are methods of assaying for a target nucleic acid as described herein that can be used for detection of a mutation in a target nucleic acid. For example, a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The detection of the signal can indicate the presence of the target nucleic acid. Sometimes, the target nucleic acid comprises a mutation. Often, the mutation is a single nucleotide mutation. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

Methods described herein can be used to identify a mutation in a target nucleic acid. The methods can be used to identify a single nucleotide mutation of a target nucleic acid that affects the expression of a gene. A mutation that affects the expression of gene can be a single nucleotide mutation of a target nucleic acid within the gene, a single nucleotide mutation of a target nucleic acid comprising RNA associated with the expression of a gene, or a target nucleic acid comprising a single nucleotide mutation of a nucleic acid associated with regulation of expression of a gene, such as an RNA or a promoter, enhancer, or repressor of the gene. Often, a status of a mutation is used to diagnose or identify diseases associated with the mutation of target nucleic acid. Detection of target nucleic acids having a mutation are applicable to a number of fields, such as clinically, as a diagnostic, in laboratories as a research tool, and in agricultural applications. Often, the mutation is a single nucleotide mutation. The mutation may result in a mutated strain of a virus, such as an influenza A or influenza B virus.

### Disease Detection

Disclosed herein are methods of assaying for a target nucleic acid as described herein that can be used for disease detection. For example, a method of assaying for a target nucleic acid (e.g., from an influenza virus) in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The detection of the signal can indicate the presence of the target nucleic acid. Sometimes, the target nucleic acid comprises a mutation. Often, the mutation is a single nucleotide mutation. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

Methods described herein can be used to identify a mutation in a target nucleic acid from a bacteria, virus, or microbe. The methods can be used to identify a mutation of a target nucleic acid that affects the expression of a gene. A mutation that affects the expression of gene can be a mutation of a target nucleic acid within the gene, a mutation of a target nucleic acid comprising RNA associated with the expression of a gene, or a target nucleic acid comprising a mutation of a nucleic acid associated with regulation of expression of a gene, such as an RNA or a promoter, enhancer, or repressor of the gene. Sometimes, a status of a target nucleic acid mutation is used to determine a pathogenicity of a bacteria, virus, or microbe or treatment resistance, such as resistance to antibiotic treatment. Often, a status of a mutation is used to diagnose or identify diseases associated with the mutation of target nucleic acids in the bacteria, virus, or microbe. Often, the mutation is a single nucleotide mutation.

### Detection as a Research Tool, Point-of-Care, or Over-the-Counter

Disclosed herein are methods of assaying for a target nucleic acid (e.g., from an influenza virus) as described herein that can be used as a research tool, and can be provided as reagent kits. For example, a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The detection of the signal can indicate the presence of the target nucleic acid. Sometimes, the target nucleic acid comprises a mutation. Often, the mutation is a single nucleotide mutation. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

The methods as described herein can be used to identify a single nucleotide mutation in a target nucleic acid. The methods can be used to identify mutation of a target nucleic acid that affects the expression of a gene. A mutation that affects the expression of gene can be a single nucleotide mutation of a target nucleic acid within the gene, a mutation of a target nucleic acid comprising RNA associated with the expression of a gene, or a target nucleic acid comprising a mutation of a nucleic acid associated with regulation of expression of a gene, such as an RNA or a promoter, enhancer, or repressor of the gene. Often, the mutation is a single nucleotide mutation.

The reagent kits or research tools can be used to detect any number of target nucleic acids, mutations, or other indications disclosed herein in a laboratory setting. Reagent kits can be provided as reagent packs for open box instrumentation.

In other embodiments, any of the systems, assay formats, Cas reporters, programmable nucleases, or other reagents can be used in a point-of-care (POC) test, which can be carried out at a decentralized location such as a hospital, POL, or clinic. These point-of-care tests can be used to diagnose any of the indications disclosed herein, such as influenza or streptococcal infections, or can be used to measure the presence or absence of a particular mutation in a target nucleic acid (e.g., EGFR). POC tests can be provided as small instruments with a consumable test card, wherein the test card is any of the assay formats (e.g., a lateral flow assay) disclosed herein.

In still other embodiments, any of the systems, assay formats, Cas reporters, programmable nucleases, or other reagents can be used in an over-the-counter (OTC), readerless format, which can be used at remote sites or at home to diagnose a range of indications, such as influenza. These indications can include influenza A, influenza B, streptococcal infections, or CT/NG infections. OTC products can include a consumable test card, wherein the test card is any of the assay formats (e.g., a lateral flow assay) disclosed herein. In an OTC product, the test card can be interpreted visually or using a mobile phone.

### Support Medium

A number of support mediums are consistent with the devices, systems, fluidic devices, kits, and methods disclosed herein. These support mediums are, for example, consistent with fluidic devices disclosed herein for detection of a target nucleic acid (e.g., from an influenza virus) within the sample, wherein the fluidic device may comprise multiple pumps, valves, reservoirs, and chambers for sample preparation, amplification of a target nucleic acid within the sample, mixing with a programmable nuclease, and detection of a detectable signal arising from cleavage of detector nucleic acids by the programmable nuclease within the fluidic system itself. These support mediums are compatible with the samples, reagents, and fluidic devices described herein for detection of an ailment, such as a a viral infection, for example an infection from influenza A or influenza B. A support medium described herein can provide a way to present the results from the activity between the reagents and the sample. The support medium provides a medium to present the detectable signal in a detectable format. Optionally, the support medium concentrates the detectable signal to a detection spot in a detection region to increase the sensitivity, specificity, or accuracy of the assay. The support mediums can present the results of the assay and indicate the presence or absence of the disease of interest targeted by the target nucleic acid. The result on the support medium can be read by eye or using a machine. The support medium helps to stabilize the detectable signal generated by the cleaved detector molecule on the surface of the support medium. In some instances, the support medium is a lateral flow assay strip. In some instances, the support medium is a PCR plate. The PCR plate can have 96 wells or 384 wells. The PCR plate can have a subset number of wells of a 96 well plate or a 384 well plate. A subset number of wells of a 96 well PCR plate is, for example, 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, or 95 wells. For example, a PCR subset plate can have 4 wells wherein a well is the size of a well from a 96 well PCR plate (e.g., a 4 well PCR subset plate wherein the wells are the size of a well from a 96 well PCR plate). A subset number of wells of a 384 well PCR plate is, for example, 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, or 380 wells. For example, a PCR subset plate can have 20 wells wherein a well is the size of a well from a 384 well PCR plate (e.g., a 20 well PCR subset plate wherein the wells are the size of a well from a 384 well PCR plate). The PCR plate or PCR subset plate can be paired with a fluorescent light reader, a visible light reader, or other imaging device. Often, the imaging device is a digital camera, such a digital camera on a mobile device. The mobile device may have a software program or a mobile application that can capture an image of the PCR plate or PCR subset plate, identify the assay being performed, detect the individual wells and the sample therein, provide image properties of the individuals wells comprising the assayed sample, analyze the image properties of the contents of the individual wells, and provide a result.

The support medium has at least one specialized zone or region to present the detectable signal. The regions comprise at least one of a sample pad region, a nucleic acid amplification region, a conjugate pad region, a detection region, and a collection pad region. In some instances, the regions are overlapping completely, overlapping partially, or in series and in contact only at the edges of the regions, where the regions are in fluid communication with its adjacent regions. In some instances, the support medium has a sample pad located upstream of the other regions; a conjugate pad region having a means for specifically labeling the detector moiety; a detection region located downstream from sample pad; and at least one matrix which defines a flow path in fluid connection with the sample pad. In some instances, the support medium has an extended base layer on top of which the various zones or regions are placed. The extended base layer may provide a mechanical support for the zones.

Described herein are sample pad that provide an area to apply the sample to the support medium. The sample may be applied to the support medium by a dropper or a pipette on top of the sample pad, by pouring or dispensing the sample on top of the sample pad region, or by dipping the sample pad into a reagent chamber holding the sample. The sample can be applied to the sample pad prior to reaction with the reagents when the reagents are placed on the support medium or be reacted with the reagents prior to application on the sample pad. The sample pad region can transfer the reacted reagents and sample into the other zones of the support medium. Transfer of the reacted reagents and sample may be by capillary action, diffusion, convection or active transport aided by a pump. In some cases, the support medium is integrated with or overlayed by microfluidic channels to facilitate the fluid transport.

The dropper or the pipette may dispense a predetermined volume. In some cases, the predetermined volume may range from about 1 μl to about 1000 μl, about 1 μl to about 500 μl, about 1 μl to about 100 μl, or about 1 μl to about 50 μl. In some cases, the predetermined volume may be at least 1 μl, 2 μl, 3 μl, 4 μl, 5 μl, 6 μl, 7 μl, 8 μl, 9 μl, 10 μl, 25 μl, 50 μl, 75 μl, 100 μl, 250 μl, 500 μl, 750 μl, or 1000 μl. The predetermined volume may be no more than 5 μl, 10 μl, 25 μl, 50 μl, 75 μl, 100 μl, 250 μl, 500 μl, 750 μl, or 1000 μl. The dropper or the pipette may be disposable or be single-use.

Optionally, a buffer or a fluid may also be applied to the sample pad to help drive the movement of the sample along the support medium. In some cases, the volume of the buffer or the fluid may range from about 1 μl to about 1000 μl, about 1 μl to about 500 μl, about 1 μl to about 100 μl, or about 1 μl to about 50 μl. In some cases, the volume of the buffer or the fluid may be at least 1 μl, 2 μl, 3 μl, 4 μl, 5 μl, 6 μl, 7 μl, 8 μl, 9 μl, 10 μl, 25 μl, 50 μl, 75 μl, 100 μl, 250 μl, 500 μl, 750 μl, or 1000 μl. The volume of the buffer or the fluid may be no more than than 5 μl, 10 μl, 25 μl, 50 μl, 75 μl, 100 μl, 250 μl, 500 μl, 750 μl, or 1000 μl. In some cases, the buffer or fluid may have a ratio of the sample to the buffer or fluid of at least 1:1, 1:2, 1:3, 1:4, 1:5, 1:6, 1:7, 1:8, 1:9, or 1:10.

The sample pad can be made from various materials that transfer most of the applied reacted reagents and samples to the subsequent regions. The sample pad may comprise cellulose fiber filters, woven meshes, porous plastic membranes, glass fiber filters, aluminum oxide coated membranes, nitrocellulose, paper, polyester filter, or polymer-based matrices. The material for the sample pad region may be hydrophilic and have low non-specific binding. The material for the sample pad may range from about 50 μm to about 1000 μm, about 50 μm to about 750 μm, about 50 μm to about 500 μm, or about 100 μm to about 500 μm.

The sample pad can be treated with chemicals to improve the presentation of the reaction results on the support medium. The sample pad can be treated to enhance extraction of nucleic acid in the sample, to control the transport of the reacted reagents and sample or the conjugate to other regions of the support medium, or to enhance the binding of the cleaved detection moiety to the conjugate binding molecule on the surface of the conjugate or to the capture molecule in the detection region. The chemicals may comprise detergents, surfactants, buffers, salts, viscosity enhancers, or polypeptides. In some instances, the chemical comprises bovine serum albumin.

Described herein are conjugate pads that provide a region on the support medium comprising conjugates coated on its surface by conjugate binding molecules that can bind to the detector moiety from the cleaved detector molecule or to the control molecule. The conjugate pad can be made from various materials that facilitate binding of the conjugate binding molecule to the detection moiety from cleaved detector molecule and transfer of most of the conjugate-bound detection moiety to the subsequent regions. The conjugate pad may comprise the same material as the sample pad or other zones or a different material than the sample pad. The conjugate pad may comprise glass fiber filters, porous plastic membranes, aluminum oxide coated membranes, paper, cellulose fiber filters, woven meshes, polyester filter, or polymer-based matrices. The material for the conjugate pad region may be hydrophilic, have low non-specific binding, or have consistent fluid flow properties across the conjugate pad. In some cases, the material for the conjugate pad may range from about 50 μm to about 1000 μm, about 50 μm to about 750 μm, about 50 μm to about 500 μm, or about 100 μm to about 500 μm.

Further described herein are conjugates that are placed on the conjugate pad and immobilized to the conjugate pad until the sample is applied to the support medium. The conjugates may comprise a nanoparticle, a gold nanoparticle, a latex nanoparticle, a quantum dot, a chemiluminescent nanoparticle, a carbon nanoparticle, a selenium nanoparticle, a fluorescent nanoparticle, a liposome, or a dendrimer. The surface of the conjugate may be coated by a conjugate binding molecule that binds to the detection moiety from the cleaved detector molecule.

The conjugate binding molecules described herein coat the surface of the conjugates and can bind to detection moiety. The conjugate binding molecule binds selectively to the detection moiety cleaved from the detector nucleic acid. Some suitable conjugate binding molecules comprise an antibody, a polypeptide, or a single stranded nucleic acid. In some cases, the conjugate binding molecule binds a dye and a fluorophore. Some such conjugate binding molecules that bind to a dye or a fluorophore can quench their signal. In some cases, the conjugate binding molecule is a monoclonal antibody. In some cases, an antibody, also referred to as an immunoglobulin, includes any isotype, variable regions, constant regions, Fc region, Fab fragments, F(ab′)2 fragments, and Fab′ fragments. Alternatively, the conjugate binding molecule is a non-antibody compound that specifically binds the detection moiety. Sometimes, the conjugate binding molecule is a polypeptide that can bind to the detection moiety. Sometimes, the conjugate binding molecule is avidin or a polypeptide that binds biotin. Sometimes, the conjugate binding molecule is a detector moiety binding nucleic acid.

The diameter of the conjugate may be selected to provide a desired surface to volume ratio. In some instances, a high surface area to volume ratio may allow for more conjugate binding molecules that are available to bind to the detection moiety per total volume of the conjugates. In some cases, the diameter of the conjugate may range from about 1 nm to about 1000 nm, about 1 nm to about 500 nm, about 1 nm to about 100 nm, or about 1 nm to about 50 nm. In some cases, the diameter of the conjugate may be at least 1 nm, 2 nm, 3 nm, 4 nm, 5 nm, 6 nm, 7 nm, 8 nm, 9 nm, 10 nm, 15 nm, 20 nm, 25 nm, 30 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 200 nm, 300 nm, 400 nm, 500 nm, 600 nm, 700 nm, 800 nm, 900 nm, or 1000 nm. In some cases, the diameter of the conjugate may be no more than 1 nm, 2 nm, 3 nm, 4 nm, 5 nm, 6 nm, 7 nm, 8 nm, 9 nm, 10 nm, 15 nm, 20 nm, 25 nm, 30 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 200 nm, 300 nm, 400 nm, 500 nm, 600 nm, 700 nm, 800 nm, 900 nm, or 1000 nm.

The ratio of conjugate binding molecules to the conjugates can be tailored to achieve desired binding properties between the conjugate binding molecules and the detection moiety. In some instances, the molar ratio of conjugate binding molecules to the conjugates is at least 1:1, 1:5, 1:10, 1:20, 1:30, 1:40, 1:50, 1:60, 1:70, 1:80, 1:90, 1:100, 1:110, 1:120, 1:130, 1:140, 1:150, 1:160, 1:170, 1:180, 1:190, 1:200, 1:250, 1:300, 1:350, 1:400, 1:450, or 1:500. In some instances, the mass ratio of conjugate binding molecules to the conjugates is at least 1:1, 1:5, 1:10, 1:20, 1:30, 1:40, 1:50, 1:60, 1:70, 1:80, 1:90, 1:100, 1:110, 1:120, 1:130, 1:140, 1:150, 1:160, 1:170, 1:180, 1:190, 1:200, 1:250, 1:300, 1:350, 1:400, 1:450, or 1:500. In some instances, the number of conjugate binding molecules per conjugate is at least 1, 10, 50, 100, 500, 1000, 5000, or 10000.

The conjugate binding molecules can be bound to the conjugates by various approached. Sometimes, the conjugate binding molecule can be bound to the conjugate by passive binding. Some such passive binding comprise adsorption, absorption, hydrophobic interaction, electrostatic interaction, ionic binding, or surface interactions. In some cases, the conjugate binding molecule can be bound to the conjugate covalently. Sometimes, the covalent bonding of the conjugate binding molecule to the conjugate is facilitated by EDC/NHS chemistry or thiol chemistry.

Described herein are detection region on the support medium that provide a region for presenting the assay results. The detection region can be made from various materials that facilitate binding of the conjugate-bound detection moiety from cleaved detector molecule to the capture molecule specific for the detection moiety. The detection pad may comprise the same material as other zones or a different material than the other zones. The detection region may comprise nitrocellulose, paper, cellulose, cellulose fiber filters, glass fiber filters, porous plastic membranes, aluminum oxide coated membranes, woven meshes, polyester filter, or polymer-based matrices. Often the detection region may comprise nitrocellulose. The material for the region pad region may be hydrophilic, have low non-specific binding, or have consistent fluid flow properties across the region pad. The material for the conjugate pad may range from about 10 μm to about 1000 μm, about 10 μm to about 750 μm, about 10 μm to about 500 μm, or about 10 μm to about 300 μm.

The detection region comprises at least one capture area with a high density of a capture molecule that can bind to the detection moiety from cleaved detection molecule and at least one area with a high density of a positive control capture molecule. The capture area with a high density of capture molecule or a positive control capture molecule may be a line, a circle, an oval, a rectangle, a triangle, a plus sign, or any other shapes. In some instances, the detection region comprise more than one capture area with high densities of more than one capture molecules, where each capture area comprises one type of capture molecule that specifically binds to one type of detection moiety from cleaved detection molecule and are different from the capture molecules in the other capture areas. The capture areas with different capture molecules may be overlapping completely, overlapping partially, or spatially separate from each other. In some instances, the capture areas may overlap and produce a combined detectable signal distinct from the detectable signals generated by the individual capture areas. Usually, the positive control spot is spatially distinct from any of the detection spot.

The capture molecule described herein bind to detection moiety and immobilized in the detection spot in the detect region. Some suitable capture molecules comprise an antibody, a polypeptide, or a single stranded nucleic acid. In some cases, the capture molecule binds a dye and a fluorophore. Some such capture molecules that bind to a dye or a fluorophore can quench their signal. Sometimes, the capture molecule is an antibody that that binds to a dye or a fluorophore can quench their signal. In some cases, the capture molecule is a monoclonal antibody. In some cases, an antibody, also referred to as an immunoglobulin, includes any isotype, variable regions, constant regions, Fc region, Fab fragments, F(ab′)2 fragments, and Fab′ fragments. Alternatively, the capture molecule is a non-antibody compound that specifically binds the detection moiety. Sometimes, the capture molecule is a polypeptide that can bind to the detection moiety. In some instances, the detection moiety from cleaved detection molecule has a conjugate bound to the detection moiety, and the conjugate-detection moiety complex may bind to the capture molecule specific to the detection moiety on the detection region. Sometimes, the capture molecule is a polypeptide that can bind to the detection moiety. Sometimes, the capture molecule is avidin or a polypeptide that binds biotin. Sometimes, the capture molecule is a detector moiety binding nucleic acid.

The detection region described herein comprises at least one area with a high density of a positive control capture molecule. The positive control spot in the detection region provides a validation of the assay and a confirmation of completion of the assay. If the positive control spot is not detectable by the visualization methods described herein, the assay is not valid and should be performed again with a new system or kit. The positive control capture molecule binds at least one of the conjugate, the conjugate binding molecule, or detection moiety and is immobilized in the positive control spot in the detect region. Some suitable positive control capture molecules comprise an antibody, a polypeptide, or a single stranded nucleic acid. In some cases, the positive control capture molecule binds to the conjugate binding molecule. Some such positive control capture molecules that bind to a dye or a fluorophore can quench their signal. Sometimes, the positive control capture molecule is an antibody that that binds to a dye or a fluorophore can quench their signal. In some cases, the positive control capture molecule is a monoclonal antibody. In some cases, an antibody includes any isotype, variable regions, constant regions, Fc region, Fab fragments, F(ab′)2 fragments, and Fab′ fragments. Alternatively, the positive control capture molecule is a non-antibody compound that specifically binds the detection moiety. Sometimes, the positive control capture molecule is a polypeptide that can bind to at least one of the conjugate, the conjugate binding molecule, or detection moiety. In some instances, the conjugate unbound to the detection moiety binds to the positive control capture molecule specific to at least one of the conjugate, the conjugate binding molecule.

The kit or system described herein may also comprise a positive control sample to determine that the activity of at least one of programmable nuclease, a guide nucleic acid, or a single stranded detector nucleic acid. Often, the positive control sample comprises a target nucleic acid that binds to the guide nucleic acid. The positive control sample is contacted with the reagents in the same manner as the test sample and visualized using the support medium. The visualization of the positive control spot and the detection spot for the positive control sample provides a validation of the reagents and the assay.

The kit or system for detection of a target nucleic acid described herein further can comprises reagents protease treatment of the sample. The sample can be treated with protease, such as Protease K, before amplification or before assaying for a detectable signal. Often, a protease treatment is for no more than 15 minutes. Sometimes, the protease treatment is for no more than 1, 5, 10, 15, 20, 25, 30, or more minutes, or any value from 1 to 30 minutes.

The kit or system for detection of a target nucleic acid described herein further comprises reagents for nucleic acid amplification of target nucleic acids in the sample. Isothermal nucleic acid amplification allows the use of the kit or system in remote regions or low resource settings without specialized equipment for amplification. Often, the reagents for nucleic acid amplification comprise a recombinase, a oligonucleotide primer, a single-stranded DNA binding (SSB) protein, and a polymerase. Sometimes, nucleic acid amplification of the sample improves at least one of sensitivity, specificity, or accuracy of the assay in detecting the target nucleic acid. In some cases, the nucleic acid amplification is performed in a nucleic acid amplification region on the support medium. Alternatively or in combination, the nucleic acid amplification is performed in a reagent chamber, and the resulting sample is applied to the support medium. Sometimes, the nucleic acid amplification is isothermal nucleic acid amplification. In some cases, the nucleic acid amplification is transcription mediated amplification (TMA). Nucleic acid amplification is helicase dependent amplification (HDA) or circular helicase dependent amplification (cHDA) in other cases. In additional cases, nucleic acid amplification is strand displacement amplification (SDA). In some cases, nucleic acid amplification is by recombinase polymerase amplification (RPA). In some cases, nucleic acid amplification is by at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value from 1 to 60 minutes. Sometimes, the nucleic acid amplification reaction is performed at a temperature of around 20-45° C. In some cases, the nucleic acid amplification reaction is performed at a temperature no greater than 20° C., 25° C., 30° C., 35° C., 37° C., 40° C., 45° C., or any value from 20° C. to 45° C. In some cases, the nucleic acid amplification reaction is performed at a temperature of at least 20° C., 25° C., 30° C., 35° C., 37° C., 40° C., or 45° C., or any value from 20° C. to 45° C.

Sometimes, the total time for the performing the method described herein is no greater than 3 hours, 2 hours, 1 hour, 50 minutes, 40 minutes, 30 minutes, 20 minutes, or any value from 3 hours to 20 minutes. Often, a method of nucleic acid detection from a raw sample comprises protease treating the sample for no more than 15 minutes, amplifying (can also be referred to as pre-amplyfing) the sample for no more than 15 minutes, subjecting the sample to a programmable nuclease-mediated detection, and assaying nuclease mediated detection. The total time for performing this method, sometimes, is no greater than 3 hours, 2 hours, 1 hour, 50 minutes, 40 minutes, 30 minutes, 20 minutes, or any value from 3 hours to 20 minutes. Often, the protease treatment is Protease K. Often the amplifying is thermal cycling amplification. Sometimes the amplifying is isothermal amplification.

Described herein are collection pad region that provide a region to collect the sample that flows down the support medium. Often the collection pads are placed downstream of the detection region and comprise an absorbent material. The collection pad can increase the total volume of sample that enters the support medium by collecting and removing the sample from other regions of the support medium. This increased volume can be used to wash unbound conjugates away from the detection region to lower the background and enhance assay sensitivity. When the design of the support medium does not include a collection pad, the volume of sample analyzed in the support medium may be determined by the bed volume of the support medium. The collection pad may provide a reservoir for sample volume and may help to provide capillary force for the flow of the sample down the support medium.

The collection pad may be prepared from various materials that are highly absorbent and able to retain fluids. Often the collection pads comprise cellulose filters. In some instances, the collection pads comprise cellulose, cotton, woven meshes, polymer-based matrices. The dimension of the collection pad, usually the length of the collection pad, may be adjusted to change the overall volume absorbed by the support medium.

The support medium described herein may have a barrier around the edge of the support medium. Often the barrier is a hydrophobic barrier that facilitates the maintenance of the sample within the support medium or flow of the sample within the support medium. Usually, the transport rate of the sample in the hydrophobic barrier is much lower than through the regions of the support medium. In some cases, the hydrophobic barrier is prepared by contacting a hydrophobic material around the edge of the support medium. Sometimes, the hydrophobic barrier comprises at least one of wax, polydimethylsiloxane, rubber, or silicone.

Any of the regions on the support medium can be treated with chemicals to improve the visualization of the detection spot and positive control spot on the support medium. The regions can be treated to enhance extraction of nucleic acid in the sample, to control the transport of the reacted reagents and sample or the conjugate to other regions of the support medium, or to enhance the binding of the cleaved detection moiety to the conjugate binding molecule on the surface of the conjugate or to the capture molecule in the detection region. The chemicals may comprise detergents, surfactants, buffers, salts, viscosity enhancers, or polypeptides. In some instances, the chemical comprises bovine serum albumin. In some cases, the chemicals or physical agents enhance flow of the sample with a more even flow across the width of the region. In some cases, the chemicals or physical agents provide a more even mixing of the sample across the width of the region. In some cases, the chemicals or physical agents control flow rate to be faster or slower in order to improve performance of the assay. Sometimes, the performance of the assay is measured by at least one of shorter assay time, longer times during cleavage activity, longer or shorter binding time with the conjugate, sensitivity, specificity, or accuracy.

### Multiplexing

The devices, systems, fluidic devices, kits, and methods described herein can be multiplexed in a number of ways. These methods of multiplexing are, for example, consistent with fluidic devices disclosed herein for detection of a target nucleic acid within the sample, wherein the fluidic device may comprise multiple pumps, valves, reservoirs, and chambers for sample preparation, amplification of one or more than one sequences of target nucleic acids within the sample, mixing with a programmable nuclease, and detection of a detectable signal arising from cleavage of detector nucleic acids by the programmable nuclease within the fluidic system itself.

Methods consistent with the present disclosure include a multiplexing method of assaying for a target nucleic acid in a sample. A multiplexing method comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. As another example, multiplexing method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

Multiplexing can be either spatial multiplexing wherein multiple different target nucleic acids at the same time, but the reactions are spatially separated. Often, the multiple target nucleic acids are detected using the same programmable nuclease, but different guide nucleic acids. The multiple target nucleic acids sometimes are detected using the different programmable nucleases. Sometimes, multiplexing can be single reaction multiplexing wherein multiple different target acids are detected in a single reaction volume. Often, at least two different programmable nucleases are used in single reaction multiplexing. For example, multiplexing can be enabled by immobilization of multiple categories of detector nucleic acids within a fluidic system, to enable detection of multiple target nucleic acids within a single fluidic system. Multiplexing allows for detection of multiple target nucleic acids in one kit or system. In some cases, the multiple target nucleic acids comprise different target nucleic acids to a virus, such as an influenza virus. In some cases, the multiple target nucleic acids comprise different target nucleic acids associated withinfluenza and another disease (e.g., sepsis or a respiratory infection, such as an upper respiratory tract virus). Multiplexing for one disease increases at least one of sensitivity, specificity, or accuracy of the assay to detect the presence of the disease in the sample. In some cases, the multiple target nucleic acids comprise target nucleic acids directed to different viruses, bacteria, or pathogens responsible for more than one disease. In some cases, multiplexing allows for discrimination between multiple target nucleic acids, such as target nucleic acids that comprise different genotypes of the same bacteria or pathogen responsible for a disease, for example, for a wild-type genotype of a bacteria or pathogen and for genotype of a bacteria or pathogen comprising a mutation, such as a single nucleotide polymorphism (SNP) that can confer resistance to a treatment, such as antibiotic treatment. For example, multiplexing comprises method of assaying comprising a single assay for a microorganism species using a first programmable nuclease and an antibiotic resistance pattern in a microorganism using a second programmable nuclease. Sometimes, multiplexing allows for discrimination between multiple target nucleic acids of different influenza strains, for example, influenza A and influenza B. Often, multiplexing allows for discrimination between multiple target nucleic acids, such as target nucleic acids that comprise different genotypes, for example, for a wild-type genotype and for SNP genotype. Multiplexing for multiple viral infections provides the capability to test a panel of diseases from a single sample. For example, multiplexing for multiple diseases can be valuable in a broad panel testing of a new patient or in epidemiological surveys. Often multiplexing is used for identifying bacterial pathogens in sepsis or other diseases associated with multiple pathogens.

Furthermore, signals from multiplexing can be quantified. For example, a method of quantification for a disease panel comprises assaying for a plurality of unique target nucleic acids in a plurality of aliquots from a sample, assaying for a control nucleic acid control in a second aliquot of the sample, and quantifying a plurality of signals of the plurality of unique target nucleic acids by measuring signals produced by cleavage of detector nucleic acids compared to the signal produced in the second aliquot. Often the plurality of unique target nucleic acids are from a plurality of viruses in the sample. Sometimes the quantification of a signal of the plurality correlates with a concentration of a unique target nucleic acid of the plurality for the unique target nucleic acid of the plurality that produced the signal of the plurality. The disease panel can be for any disease, such as influenza.

The devices, systems, fluidic devices, kits, and methods described herein can be multiplexed by various configurations of the reagents and the support medium. In some cases, the kit or system is designed to have multiple support mediums encased in a single housing. Sometimes, the multiple support mediums housed in a single housing share a single sample pad. The single sample pad may be connected to the support mediums in various designs such as a branching or a radial formation. Alternatively, each of the multiple support mediums has its own sample pad. In some cases, the kit or system is designed to have a single support medium encased in a housing, where the support medium comprises multiple detection spots for detecting multiple target nucleic acids. Sometimes, the reagents for multiplexed assays comprise multiple guide nucleic acids, multiple programmable nucleases, and multiple single stranded detector nucleic acids, where a combination of one of the guide nucleic acids, one of the programmable nucleases, and one of the single stranded detector nucleic acids detects one target nucleic acid and can provide a detection spot on the detection region. In some cases, the combination of a guide nucleic acid, a programmable nuclease, and a single stranded detector nucleic acid configured to detect one target nucleic acid is mixed with at least one other combination in a single reagent chamber. In some cases, the combination of a guide nucleic acid, a programmable nuclease, and a single stranded detector nucleic acid configured to detect one target nucleic acid is mixed with at least one other combination on a single support medium. When these combinations of reagents are contacted with the sample, the reaction for the multiple target nucleic acids occurs simultaneously in the same medium or reagent chamber. Sometimes, this reacted sample is applied to the multiplexed support medium described herein.

In some cases, the combination of a guide nucleic acid, a programmable nuclease, and a single stranded detector nucleic acid configured to detect one target nucleic acid is provided in its own reagent chamber or its own support medium. In this case, multiple reagent chambers or support mediums are provided in the device, kit, or system, where one reagent chamber is designed to detect one target nucleic acid. In this case, multiple support mediums are used to detect the panel of viral infections, or other diseases of interest.

In some instances, the multiplexed devices, systems, fluidic devices, kits, and methods detect at least 2 different target nucleic acids in a single reaction. In some instances, the multiplexed devices, systems, fluidic devices, kits, and methods detect at least 3 different target nucleic acids in a single reaction. In some instances, the multiplexed devices, systems, fluidic devices, kits, and methods detect at least 4 different target nucleic acids in a single reaction. In some instances, the multiplexed devices, systems, fluidic devices, kits, and methods detect at least 5 different target nucleic acids in a single reaction. In some cases, the multiplexed devices, systems, fluidic devices, kits, and methods detect at least 6, 7, 8, 9, or 10 different target nucleic acids in a single reaction. In some instances, the multiplexed kits detect at least 2 different target nucleic acids in a single kit. In some instances, the multiplexed kits detect at least 3 different target nucleic acids in a single kit. In some instances, the multiplexed kits detect at least 4 different target nucleic acids in a single kit. In some instances, the multiplexed kits detect at least 5 different target nucleic acids in a single kit. In some instances, the multiplexed kits detect at least 6, 7, 8, 9, or 10 different target nucleic acids in a single kit.

### Housing

A support medium as described herein can be housed in a number of ways that are consistent with the devices, systems, fluidic devices, kits, and methods disclosed herein. The housing for the support medium are, for example, consistent with fluidic devices disclosed herein for detection of a target nucleic acid within the sample, wherein the fluidic device may comprise multiple pumps, valves, reservoirs, and chambers for sample preparation, amplification of a target nucleic acid within the sample, mixing with a programmable nuclease, and detection of a detectable signal arising from cleavage of detector nucleic acids by the programmable nuclease within the fluidic system itself. For example, the fluidic device may be comprise support mediums to channel the flow of fluid from one chamber to another and wherein the entire fluidic device is encased within the housing described herein. Typically, the support medium described herein is encased in a housing to protect the support medium from contamination and from disassembly. The housing can be made of more than one part and assembled to encase the support medium. In some instances, a single housing can encase more than one support medium. The housing can be made from cardboard, plastics, polymers, or materials that provide mechanical protection for the support medium. Often, the material for the housing is inert or does not react with the support medium or the reagents placed on the support medium. The housing may have an upper part which when in place exposes the sample pad to receive the sample and has an opening or window above the detection region to allow the results of the lateral flow assay to be read. The housing may have guide pins on its inner surface that are placed around and on the support medium to help secure the compartments and the support medium in place within the housing. In some cases, the housing encases the entire support medium. Alternatively, the sample pad of the support medium is not encased and is left exposed to facilitate the receiving of the sample while the rest of the support medium is encased in the housing.

The housing and the support medium encased within the housing may be sized to be small, portable, and hand held. The small size of the housing and the support medium would facilitate the transport and use of the assay in remote regions or low resource settings. In some cases, the housing has a length of no more than 30 cm, 25 cm, 20 cm, 15 cm, 10 cm, or 5 cm. In some cases, the housing has a length of at least 1 cm, 5 cm, 10 cm, 15 cm, 20 cm, 25 cm, or 30 cm. In some cases, the housing has a width of no more than 30 cm, 25 cm, 20 cm, 15 cm, 10 cm, 5 cm, 4 cm, 3 cm, 2 cm, or 1 cm. In some cases, the housing has a width of at least 1 cm, 2 cm, 3 cm, 4 cm, 5 cm, 10 cm, 15 cm, 20 cm, 25 cm, or 30 cm. In some cases, the housing has a height of no more than 10 cm, 9 cm, 8 cm, 7 cm, 6 cm, 5 cm, 4 cm, 3 cm, 2 cm, or 1 cm. In some cases, the housing has a height of at least 1 cm, 2 cm, 3 cm, 4 cm, 5 cm, 6 cm, 7 cm, 8 cm, 9 cm, or 10 cm. Typically, the housing is rectangular in shape.

The housing may comprise more than one piece. The housing may comprise an over-molding. The housing may seal a chamber, channel, compartment, or valve from the surrounding environment. The housing may be comprise sealable materials, such as polycarbonate capable of laser bonding. The housing may comprise a rigid material. The housing may comprise a flexible material. The housing may comprise connectors or adaptors. A set of connectors or adaptors may have tight tolerances. A set of connectors or adaptors may have loose tolerances.

In some instances, the housing provides additional information on the outer surface of the upper cover to facilitate the identification of the test type, visualization of the detection region, and analysis of the results. The upper outer housing may have identification label including but not limited to barcodes, QR codes, identification label, or other visually identifiable labels. In some instances, the identification label is imaged by a camera on a mobile device, and the image is analyzed to identify the disease that is being tested for. The correct identification of the test is important to accurately visualize and analyze the results. In some instances, the upper outer housing has fiduciary markers to orient the detection region to distinguish the positive control spot from the detection spots. In some instances, the upper outer housing has a color reference guide. When the detection region is imaged with the color reference guide, the detection spots, located using the fiduciary marker, can be compared with the positive control spot and the color reference guide to determine various image properties of the detection spot such as color, color intensity, and size of the spot. In some instances, the color reference guide has red, green, blue, black, and white colors. In some cases, the image of the detection spot can be normalized to at least one of the reference colors of the color reference guide, compared to at least two of the reference colors of the color reference guide, and generate a value for the detection spot. Sometimes, the comparison to at least two of the reference colors is comparison to a standard reference scale. In some instance, the image of the detection spot in some instance undergoes transformation or filtering prior to analysis. Analysis of the image properties of the detection spot can provide information regarding presence or absence of the target nucleic acid targeted by the assay and the disease associated with the target nucleic acid. In some instances, the analysis provides a qualitative result of presence or absence of the target nucleic acid in the sample. In some instances, the analysis provides a semi-quantitative or quantitative result of the level of the target nucleic acid present in the sample. Quantification may be performed by having a set of standards in spots/wells and comparing the test sample to the range of standards. A more semi-quantitative approach may be performed by calculating the color intensity of 2 spots/well compared to each other and measuring if one spot/well is more intense than the other. Sometimes, quantification is of quantification of circulating nucleic acid. The circulating nucleic acid can comprise a target nucleic acid. For example, a method of circulating nucleic acid quantification comprises assaying for a target nucleic acid of circulating nucleic acid in a first aliquot of a sample, assaying for a control nucleic acid in a second aliquot of the sample, and quantifying the target nucleic acid target in the first aliquot by measuring a signal produced by cleavage of a detector nucleic acid. Sometimes, a method of circulating RNA quantification comprises assaying for a target nucleic acid of the circulating RNA in a first aliquot of a sample, assaying for a control nucleic acid in a second aliquot of the sample, and quantifying the target nucleic acid target in the first aliquot by measuring a signal produced by cleavage of a detector nucleic acid. Often, the output comprises fluorescence/second. The reaction rate, sometimes, is log linear for output signal and target nucleic acid concentration. In some instances, the signal output is correlated with the target nucleic acid concentration. Sometimes, the circulating nucleic acid is DNA.

### Detection/Visualization Devices

A number of detection or visualization devices and methods are consistent with the devices, systems, fluidic devices, kits, and methods disclosed herein. Methods of detection/visualization are, for example, consistent with fluidic devices disclosed herein for detection of a target nucleic acid within the sample, wherein the fluidic device may comprise multiple pumps, valves, reservoirs, and chambers for sample preparation, amplification of a target nucleic acid within the sample, mixing with a programmable nuclease, and detection of a detectable signal arising from cleavage of detector nucleic acids by the programmable nuclease within the fluidic system itself. For example, the fluidic device may comprise an incubation and detection chamber or a stand-alone detection chamber, in which a colorimetric, fluorescence, electrochemical, or electrochemiluminesence signal is generated for detection/visualization. Sometimes, the signal generated for detection is a calorimetric, potentiometric, amperometric, optical (e.g., fluorescent, colorimetric, etc.), or piezo-electric signal. Often a calorimetric signal is heat produced after cleavage of the detector nucleic acids. Sometimes, a calorimetric signal is heat absorbed after cleavage of the detector nucleic acids. A potentiometric signal, for example, is electrical potential produced after cleavage of the detector nucleic acids. An amperometric signal can be movement of electrons produced after the cleavage of detector nucleic acid. Often, the signal is an optical signal, such as a colorimetric signal or a fluorescence signal. An optical signal is, for example, a light output produced after the cleavage of the detector nucleic acids. Sometimes, an optical signal is a change in light absorbance between before and after the cleavage of detector nucleic acids. Often, a piezo-electric signal is a change in mass between before and after the cleavage of the detector nucleic acid. Sometimes, the detector nucleic acid is protein-nucleic acid. Often, the protein-nucleic acid is an enzyme-nucleic acid. The detection/visualization can be analyzed using various methods, as further described below. The results from the detection region from a completed assay can be visualized and analyzed in various ways. In some cases, the positive control spot and the detection spot in the detection region is visible by eye, and the results can be read by the user. In some cases, the positive control spot and the detection spot in the detection region is visualized by an imaging device. Often, the imaging device is a digital camera, such a digital camera on a mobile device. The mobile device may have a software program or a mobile application that can capture an image of the support medium, identify the assay being performed, detect the detection region and the detection spot, provide image properties of the detection spot, analyze the image properties of the detection spot, and provide a result. Alternatively or in combination, the imaging device can capture fluorescence, ultraviolet (UV), infrared (IR), or visible wavelength signals. The imaging device may have an excitation source to provide the excitation energy and captures the emitted signals. In some cases, the excitation source can be a camera flash and optionally a filter. In some cases, the imaging device is used together with an imaging box that is placed over the support medium to create a dark room to improve imaging. The imaging box can be a cardboard box that the imaging device can fit into before imaging. In some instances, the imaging box has optical lenses, mirrors, filters, or other optical elements to aid in generating a more focused excitation signal or to capture a more focused emission signal. Often, the imaging box and the imaging device are small, handheld, and portable to facilitate the transport and use of the assay in remote or low resource settings.

In some cases, detection or visualization may comprise the production of light by a diode. In some cases, a diode may produce visible light. In some cases, a diode may produce infrared light. In some cases, a diode may produce ultraviolet light. In some cases, a diode may be capable of producing different wavelengths or spectra of light. A diode may produce light over a broad or narrow spectrum. A diode may produce white light covering a large portion of the visible spectrum. A diode may produce a specific wavelength of light (e.g., a roughly Gaussian or Lorentzian wavelength vs intensity profile centered around a particular wavelength). In some cases, the bandwidth of light produced by a diode may be defined as the full width at half maximum intensity of a Gaussian-like or Lorentzian-like band. Some diodes produce light with narrow emission bandwidths. A diode may produce light with less than a 1 nm bandwidth. A diode may produce light with less than a 5 nm bandwidth. A diode may produce light with less than a 10 nm bandwidth. A diode may produce light with less than a 20 nm bandwidth. A diode may produce light with less than a 30 nm bandwidth. A diode may produce light with less than a 50 nm bandwidth. A diode may produce light with less than a 100 nm bandwidth. A diode may produce light with less than a 150 nm bandwidth. A diode may produce light with less than a 200 nm bandwidth.

In some cases, detection or visualization may comprise light detection by a diode (e.g., a photodiode). The current produced by a diode may be used to determine characteristics of light absorbed, including polarization, wavelength, intensity, direction traveled, point of origin, or any combination thereof. In some cases, detection or visualization may comprise light detection by a camera (e.g., a charge coupled device (CCD) detector) or a metal—oxide—semiconductor (MOS) detector). A detector (e.g., a photodiode, a CCD detector, or a MOS detector) may be configured to detect a bandwidth of light. In some cases, the bandwidth of light detected by a detector may be defined as the full width at half maximum intensity of a Gaussian-like or Lorentzian-like band. In some cases, the bandwidth of light detected by a detector may be narrowed by an emission filter positioned between the sample and the detector. The emission filter may be a long pass filter. The emission filter may be bandpass filter. The emission filter may be a notch filter. In some embodiments, the bandwidth of light detected by the detector may be less than about 300 nm, less than about 200 nm, less than about 100 nm, less than about 75 nm, less than about 50 nm, less than about 40 nm, less than about 30 nm, less than about 20 nm, less than about 10 nm, or less than about 5 nm.

In some cases, a diode array may be used to excite and detect fluorescence from a sample. In some cases, a device may comprise a light producing diode and detector diode positioned to illuminate and detect light from a particular portion of a sample. In some cases, a device may comprise a light producing diode and detector diode positioned to illuminate and detect light from a particular sample compartment or chamber.

The assay described herein can be visualized and analyzed by a mobile application (app) or a software program. Using the graphic user interface (GUI) of the app or program, an individual can take an image of the support medium, including the detection region, barcode, reference color scale, and fiduciary markers on the housing, using a camera on a mobile device. The program or app reads the barcode or identifiable label for the test type, locate the fiduciary marker to orient the sample, and read the detectable signals, compare against the reference color grid, and determine the presence or absence of the target nucleic acid, which indicates the presence of the gene, virus, or the agent responsible for the disease. The mobile application can present the results of the test to the individual. The mobile application can store the test results in the mobile application. The mobile application can communicate with a remote device and transfer the data of the test results. The test results can be viewable remotely from the remote device by another individual, including a healthcare professional. A remote user can access the results and use the information to recommend action for treatment, intervention, clean up of an environment.

### Manufacturing

The support medium may be assembled with a variety of materials and reagents. Reagents may be dispensed or coated on to the surface of the material for the support medium. The material for the support medium may be laminated to a backing card, and the backing card may be singulated or cut into individual test strips. The device may be manufactured by completely manual, batch-style processing; or a completely automated, in-line continuous process; or a hybrid of the two processing approaches. The batch process may start with sheets or rolls of each material for the support medium. Individual zones of the support medium may be processed independently for dispensing and drying, and the final support medium may be assembled with the independently prepared zones and cut. The batch processing scheme may have a lower cost of equipment, and a higher labor cost than more automated in-line processing, which may have higher equipment costs. In some instances, batch processing may be preferred for low volume production due to the reduced capital investment. In some instances, automated in-line processing may be preferred for high volume production due to reduced production time. Both approaches may be scalable to production level.

In some instances, the support mediums are prepared using various instruments, including an XYZ-direction motion system with dispensers, impregnation tanks, drying ovens, a manual or semi-automated laminator, and cutting methods for reducing roll or sheet stock to appropriate lengths and widths for lamination. For dispensing the conjugate binding molecules for the conjugate zone and capture molecules for the detection zones, an XYZ-direction motion system with dispensers may be used. In some embodiments, the dispenser may dispense by a contact method or a non-contact method.

In automated or semi-automated preparation of the support medium, the support medium may be prepared from rolls of membranes for each region that are ordered into the final assembled order and unfurled from the rolls. For example, the membranes can be ordered from sample pad region to collection pad region from left to right with one membrane corresponding to a region on the support medium, all onto an adhesive cardstock. The dispenser places the reagents, conjugates, detection molecules, and other treatments for the membrane onto the membrane. The dispensed fluids are dried onto the membranes by heat, in a low humidity chamber, or by freeze drying to stabilize the dispensed molecules. The membranes are cut into strips and placed into the housing and packaged.

### Detection of a Target Nucleic Acid in a Fluidic Device

Disclosed herein are various fluidic devices for detection of a target nucleic acid of interest in a biological sample. The fluidic devices described in detail below can be used to monitor the reaction of target nucleic acids in samples with a programmable nuclease, thereby allowing for the detection of said target nucleic acid. All samples and reagents disclosed herein are compatible for use with a fluidic device disclosed below. Any programmable nuclease, such as any Cas nuclease described herein, are compatible for use with a fluidic device disclosed below. Support mediums and housing disclosed herein are also compatible for use in conjunction with the fluidic devices disclosed below. Multiplexing detection, as described throughout the present disclosure, can be carried out within the fluidic devices disclosed herein. Compositions and methods for detection and visualization disclosed herein are also compatible for use within the below described fluidic systems.

In the below described fluidic systems, any programmable nuclease (e.g., CRISPR-Cas) reaction can be monitored. For example, any programmable nuclease disclosed herein can be used to cleave the reporter molecules to generate a detection signal. In some cases, the programmable nuclease is Cas13. Sometimes the Cas13 is Cas13a, Cas13b, Cas13c, Cas13d, or Cas13e. In some cases, the programmable nuclease is Mad7 or Mad2. In some cases, the programmable nuclease is Cas12. Sometimes the Cas12 is Cas12a, Cas12b, Cas12c, Cas12d, or Cas12e. In some cases, the programmable nuclease is Csm1, Cas9, C2c4, C2c8, C2c5, C2c10, C2c9, or CasZ. Sometimes, the Csm1 is also called smCms1, miCms1, obCms1, or suCms1. Sometimes Cas13a is also called C2c2. Sometimes CasZ is also called Cas14a, Cas14b, Cas14c, Cas14d, Cas14e, Cas14f, Cas14g, or Cas14h. Sometimes, the programmable nuclease is a type V CRISPR-Cas system. In some cases, the programmable nuclease is a type VI CRISPR-Cas system. Sometimes the programmable nuclease is a type III CRISPR-Cas system. In some cases, the programmable nuclease is from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pint), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a.

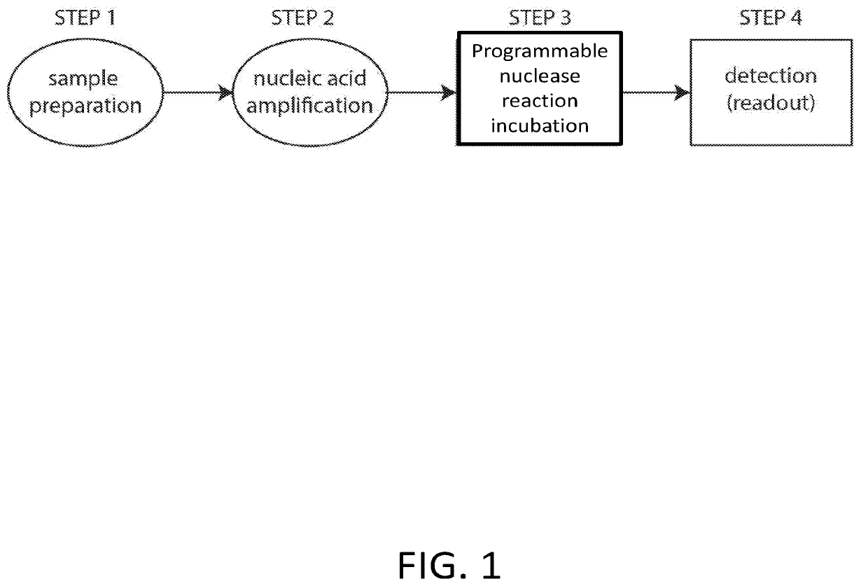

A workflow of a method for detecting a target nucleic acid in a sample within a fluidic device can include sample preparation, nucleic acid amplification, incubation with a programmable nuclease, and/or detection (readout). FIG. 1 shows a schematic illustrating a workflow of a programmable nuclease reaction. Step 1 shown in the workflow is sample preparation, Step 2 shown in the workflow is nucleic acid amplification. Step 3 shown in the workflow is programmable nuclease incubation. Step 4 shown in the workflow is detection (readout). Non-essential steps are shown as oval circles. Steps 1 and 2 are optional, and steps 3 and 4 can occur concurrently, if incubation and detection of programmable nuclease activity are within the same chamber. Sample preparation and amplification can be carried out within a fluidic device described herein or, alternatively, can be carried out prior to introduction into the fluidic device. As mentioned above, sample preparation of any nucleic acid amplification are optional, and can be excluded. In further cases, programmable nuclease reaction incubation and detection (readout) can be performed sequentially (one after another) or concurrently (at the same time). In some embodiments, sample preparation and/or amplification can be performed within a first fluidic device and then the sample can be transferred to a second fluidic device to carry out Steps 3 and 4 and, optionally, Step 2.

Workflows and systems compatible with the compositions and methods provided herein include one-pot reactions and two-pot reactions. In a one-pot reaction, amplification, reverse transcription, amplification and reverse transcription, or amplification and in vitro transcription, and detection can be carried out simultaneously in one chamber. In other words, in a one-pot reaction, any combination of reverse transcription, amplification, and in vitro transcription can be performed in the same reaction as detection. In a two-pot reaction, any combination of reverse transcription, amplification, and in vitro transcription can be performed in a first reaction, followed by detection in a second reaction. The one-pot or two-pot reactions can be carried out in any of the chambers of the devices disclosed herein.

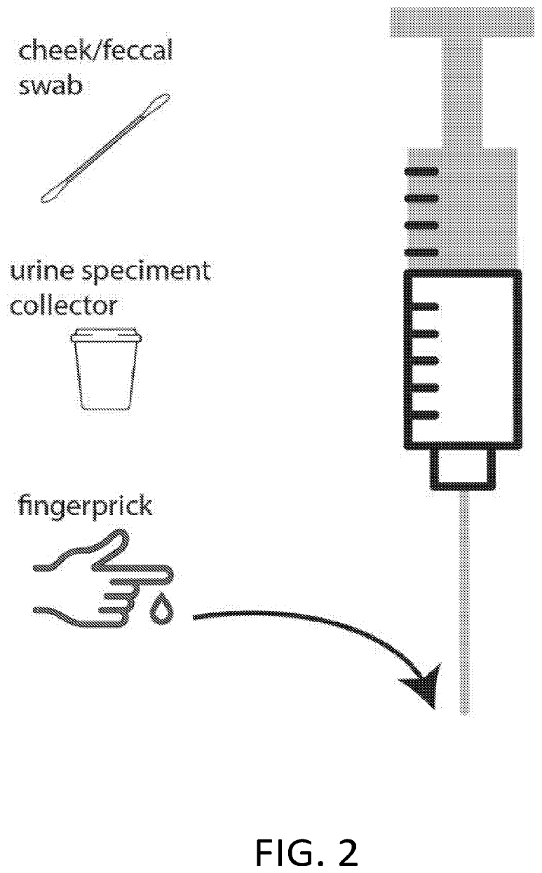

A fluidic device for sample preparation can be referred to as a filtration device. In some embodiments, the filtration device for sample preparation resembles a syringe or, comprises, similar functional elements to a syringe. For example, a functional element of the filtration device for sample preparation includes a narrow tip for collection of liquid samples. Liquid samples can include blood, saliva, urine, or any other biological fluid. Liquid samples can also include liquid tissue homogenates. The tip, for collection of liquid samples, can be manufactured from glass, metal, plastic, or other biocompatible materials. The tip may be replaced with a glass capillary that may serve as a metering apparatus for the amount of biological sample added downstream to the fluidic device. For some samples, e.g., blood, the capillary may be the only fluidic device required for sample preparation. Another functional element of the filtration device for sample preparation may include a channel that can carry volumes from nL to mL, containing lysis buffers compatible with the programmable nuclease reaction downstream of this process. The channel may be manufactured from metal, plastic, or other biocompatible materials. The channel may be large enough to hold an entire fecal, buccal, or other biological sample collection swab. The filtration device may further contain a solution of reagents that will lyse the cells in each type of samples and release the nucleic acids so that they are accessible to the programmable nuclease. Active ingredients of the solution may be chaotropic agents, detergents, salts, and can be of high osmolality, ionic strength and pH. Chaotropic agents or chaotropes are substances that disrupt the three-dimensional structure in macromolecules such as proteins, DNA, or RNA. One example protocol comprises a 4 M guanidinium isothiocyanate, 25 mM sodium citrate. 2H₂O, 0.5% (w/v) sodium lauryl sarcosinate, and 0.1 M β-mercaptoethanol), but numerous commercial buffers for different cellular targets may also be used. Alkaline buffers may also be used for cells with hard shells, particularly for environmental samples. Detergents such as sodium dodecyl sulphate (SDS) and cetyl trimethylammonium bromide (CTAB) may also be implemented to chemical lysis buffers. Cell lysis may also be performed by physical, mechanical, thermal or enzymatic means, in addition to chemically-induced cell lysis mentioned previously. The device may include more complex architecture depending on the type of sample, such as nanoscale barbs, nanowires, sonication capability in a separate chamber of the device, integrated laser, integrated heater, for example, a Peltier-type heater, or a thin-film planar heater, and/or microcapillary probes for electrical lysis. Any samples described herein can be used in this workflow. For example samples may include liquid samples collected from a subject being tested for a condition of interest. FIG. 2 shows an example fluidic, or filtration, device for sample preparation that may be used in Step 1 of the workflow schematic of 1. The sample preparation fluidic device shown in this figure can process different types of biological sample: finger-prick blood, urine or swabs with fecal, cheek or other collection.

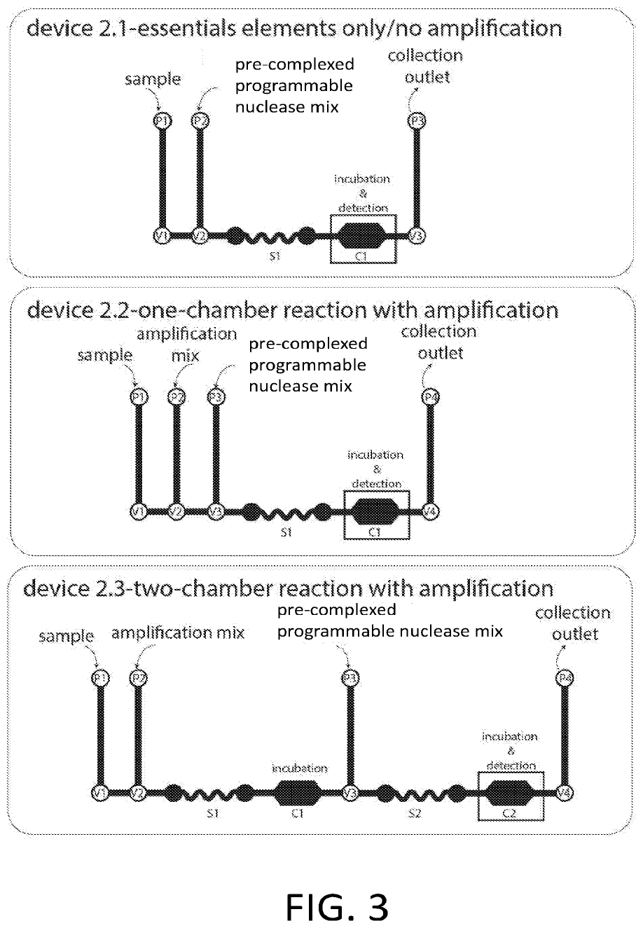

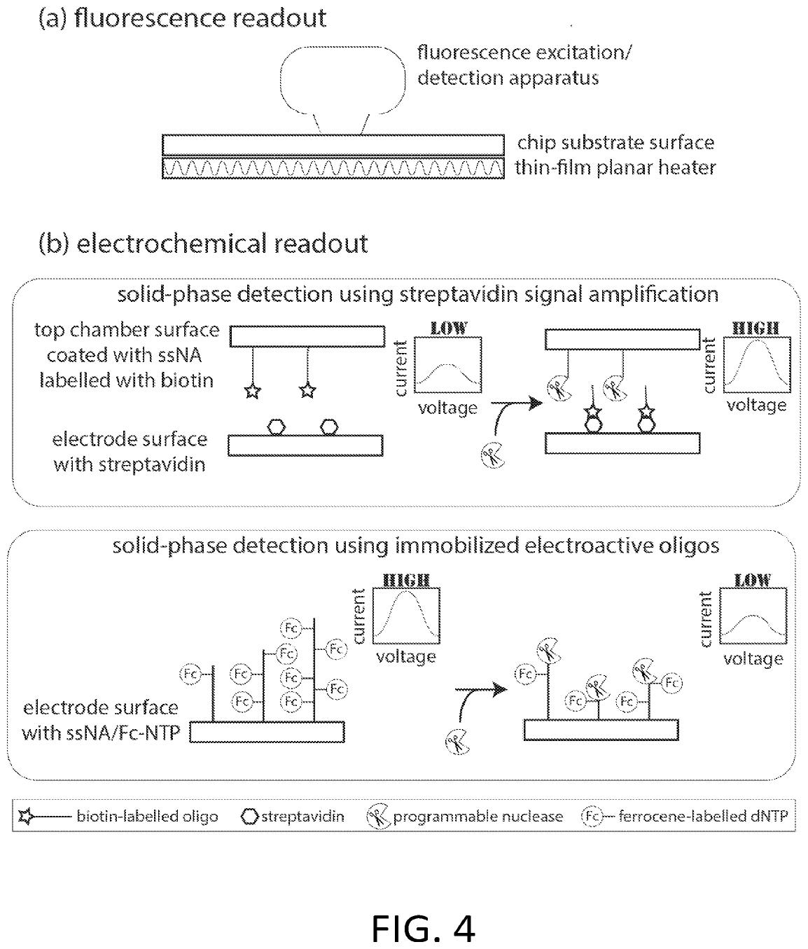

A fluidic device may be used to carry out any one of, or any combination of, Steps 2-4 of FIG. 1 (nucleic acid amplification, programmable nuclease reaction incubation, detection (readout)). FIG. 3 shows an example fluidic device for a programmable nuclease reaction with a fluorescence or electrochemical readout that may be used in Step 2 to Step 4 of the workflow schematic of FIG. 1. This figure shows that the device performs three iterations of Steps 2 through 4 of the workflow schematic of FIG. 1. At top, is one variation of this fluidic device, which performs the programmable nuclease reaction incubation and detection (readout) steps, but not amplification. Shown in the middle is another variation of said fluidic device, comprising a one-chamber reaction with amplification. Shown at bottom is yet another variation of the fluidic device, comprising a two-chamber reaction with amplification. An exploded view diagram summarizing the fluorescence and electrochemical processes that may be used for detection of the reaction are shown in FIG. 4.

A fluidic device may comprise a plurality of chambers and types of chambers. A fluidic device may comprise a plurality of chambers configured to contain a sample with reagents and in conditions conducive to a particular type of reaction. Such a chamber may be designed to facilitate detection of a reaction or a reaction species (e.g., by having transparent surfaces so that the contents of the chamber can be monitored by an external fluorimeter, or by having electrodes capable of potentiometric analysis). A fluidic device may comprise an amplification chamber, which can be designed to contain a sample and reagents in conditions (e.g., temperature) suitable for an amplification reaction. A fluidic device may comprise a detection chamber, which may be designed to contain a sample with reagents in conditions suitable for a detection reaction (e.g., a colorimetric reaction or a DETECTR reaction). A fluidic device may also comprise chambers designed to store or transfer reagents. For example, a fluidic device may comprise an amplification reagent chamber designed to hold reagents for an amplification reaction (e.g., LAMP) or a detection reagent chamber designed to hold reagents for a reaction capable of detecting the presence or absence of a species (e.g., a DETECTR reaction). A fluidic device may comprise a chamber configured for multiple purposes (e.g., a chamber may be configured for storing a reagent, containing two types of samples for two separate types of reactions, and facilitating fluorescence detection).

A fluidic device may comprise a sample inlet (the term ‘sample inlet’ is herein used interchangeably with sample inlet port and sample collection port) that leads to an internal space within the fluidic device, such as a chamber or fluidic channel. A sample inlet may lead to a chamber within the fluidic device. A sample inlet may be capable of sealing. A sample inlet may be sealed such that fluid is prevented from passing through the sample inlet. In some cases, a sample inlet seals around a second apparatus designed to deliver a sample, thus sealing the sample inlet from the surrounding environment. For example, a sample inlet may be capable of sealing around a swab or syringe. A sample inlet may also be configured to accommodate a cap or other mechanism that covers or seals the A sample inlet may comprise a bendable or breakable component. For example, a sample inlet may comprise a seal that breaks upon sample insertion. In some cases, a seal within a sample inlet releases reagents upon breaking. A sample inlet may comprise multiple chambers or compartments. For example, a sample inlet may comprise an upper compartment and a lower compartment separated by a breakable plastic seal. The seal may break upon sample insertion, releasing contents (e.g., lysis buffer or amplification buffer) from the upper container into the lower container, where it may mix with the sample and elute into a separate compartment (e.g., a sample compartment) within the fluidic device.

In some embodiments, the fluidic device may be a pneumatic device. The pneumatic device may comprise one or more sample chambers connected to one or more detection chambers by one or more pneumatic valves. Optionally, the pneumatic device may further comprise one or more amplification chamber between the one or more sample chambers and the one or more detection chambers. The one or more amplification chambers may be connected to the one or more sample chambers and the one or more detection chambers by one or more pneumatic valves. A pneumatic valve may be made from PDMS, or any other suitable material. A pneumatic valve may comprise a channel perpendicular to a microfluidic channel connecting the chambers and allowing fluid to pass between chambers when the valve is open. In some embodiments, the channel deflects downward upon application of air pressure through the channel perpendicular to the microfluidic channel. In some embodiments, the fluidic device may be a sliding valve device. The sliding valve device may comprise a sliding layer with one or more channels and a fixed layer with one or more sample chambers and one or more detection chambers. Optionally, the fixed layer may further comprise one or more amplification chambers. In some embodiments, the sliding layer is the upper layer and the fixed layer is the lower layer. In other embodiments, the sliding layer is the lower layer and the fixed layer is the upper layer. The sliding valve device may further comprise one or more of a side channel with an opening aligned with an opening in the sample chamber, a side channel with an opening aligned with an opening in the amplification chamber, or a side channel with an opening aligned with the opening in the detection chamber. In some embodiments the side channels are connected to a mixing chamber to allow transfer of fluid between the chambers. In some embodiments, the sliding valve device comprises a pneumatic pump for mixing, aspirating, and dispensing fluid in the device.

In some embodiments, a fluidic device may comprise a sliding valve. A sliding valve may be capable of adopting multiple positions, that connect different channels or compartments in a device. In some cases, a sliding device comprises multiple sets of channels that can simultaneously connect multiple different channels or compartments. For example a device that comprises 10 amplification chambers, 10 reagent chambers, and 1 sample chamber may comprise a sliding valve that can adopt a first position connecting the sample chamber to the 10 amplification chambers through 10 separate channels, and a second position that may separately connect the 10 amplification chambers to the 10 reagent chambers. A sliding valve may be capable of automated control by a device or computer. A sliding valve may comprise a transfer fluidic channel, which can have a first end that is open to a first chamber or fluidic channel and a second end that is blocked when the sliding valve is in a first position, and can have the first end blocked and the second end open to a second chamber or fluidic channel when the sliding valve is in a second position. A sliding valve may be designed to combine the flow from two or more chambers or channels into a single chamber or channel. A sliding valve may be designed to divide the flow from a single chamber or channel into two or more separate chambers or fluidic channels.

The chip (also referred to as fluidic device) may be manufactured from a variety of different materials. Exemplary materials that may be used include plastic polymers, such as poly-methacrylate (PMMA), cyclic olefin polymer (COP), cyclic olefin copolymer (COC), polyethylene (PE), high-density polyethylene (HDPE), polypropylene (PP); glass; and silicon. Features of the chip may be manufactured by various processes. For example, features may be (1) embossed using injection molding, (2) micro-milled or micro-engraved using computer numerical control (CNC) micromachining, or non-contact laser drilling (by means of a CO2 laser source); (3) additive manufacturing, and/or (4) photolithographic methods. A chip may comprise a material or combination of materials that thermally isolate different portions of the chip (e.g., two fluidic channels or reaction chambers may be thermally isolated by intervening material between them).

A design may include a plurality of input ports operated by a plurality of pumps. For example, the design may include up to three (3) input ports operated by three (3) pumps, labelled on FIG. 3 as P1-P3. The pumps may be operated by external syringe pumps using low pressure or high pressure. The pumps may be passive, and/or active (pneumatic, piezoelectric, Braille pin, electroosmotic, acoustic, gas permeation, or other).

The ports may be connected to pneumatic pressure pumps, air or gas may be pumped into the microfluidic channels to control the injection of fluids into the fluidic device. At least three reservoirs may be connected to the device, each containing buffered solutions of: (1) sample, which may be a solution containing purified nucleic acids processed in a separate fluidic device, or neat sample (blood, saliva, urine, stool, and/or sputum); (2) amplification mastermix, which varies depending on the method used, wherein the method may include any of loop-mediated isothermal amplification (LAMP), strand displacement amplification (SDA), recombinase polymerase amplification (RPA), helicase dependent amplification (HDA), multiple displacement amplification (MDA), rolling circle amplification (RCA), and nucleic acid sequence-based amplification (NASBA), transcription mediated amplification (TMA), circular helicase dependent amplification (cHDA), exponential amplification reaction (EXPAR), ligase chain reaction (LCR), simple method amplifying RNA targets (SMART), single primer isothermal amplification (SPIA), hinge-initiated primer-dependent amplification of nucleic acids (HIP), nicking enzyme amplification reaction (NEAR), or improved multiple displacement amplification (IMDA); and (3) pre-complexed programmable nuclease mix, which includes one or more programmable nuclease and guide oligonucleotides. The method of nucleic acid amplification may also be polymerase chain reaction (PCR), which includes cycling of the incubation temperature at different levels, hence is not defined as isothermal. Often, the reagents for nucleic acid amplification comprise a recombinase, a oligonucleotide primer, a single-stranded DNA binding (SSB) protein, and a polymerase. Sometimes, nucleic acid amplification of the sample improves at least one of sensitivity, specificity, or accuracy of the assay in detecting the target nucleic acid. In some cases, the nucleic acid amplification is performed in a nucleic acid amplification region on the support medium. Alternatively or in combination, the nucleic acid amplification is performed in a reagent chamber, and the resulting sample is applied to the support medium. Sometimes, the nucleic acid amplification is isothermal nucleic acid amplification. Complex formation of a nuclease with guides (a programmable nuclease) and reporter probes may occur off the chip. An additional port for output of the final reaction products is depicted at the end of the fluidic path, and is operated by a similar pump, as the ones described for P1-P3. The reactions product can be, thus, collected for additional processing and/or characterization, e.g., sequencing.

A device may comprise a plurality of chambers, fluidic channels and valves. A device may comprise multiple types of chambers, fluidic channels, valves, or any combination thereof. A device may comprise different numbers of chambers, fluidic channels, and valves. For example, a device may comprise one sample chamber, a rotating valve connecting the sample chamber to 10 separate amplification reaction chambers, and two sliding valves controlling flow from the 10 amplification reaction chambers into 30 separate Detection chambers. A rotating valve may connect 2 or more chambers or fluidic channels. A rotating valve may connect 3 or more chambers or fluidic channels. A rotating valve may connect 4 or more chambers or fluidic channels. A rotating valve may connect 5 or more chambers or fluidic channels. A rotating valve may connect 8 or more chambers or fluidic channels. A rotating valve may connect 10 or more chambers or fluidic channels. A rotating valve may connect 15 or more chambers or fluidic channels. A rotating valve may connect 20 or more chambers or fluidic channels.

A fluidic device may comprise a plurality of channels. A fluidic device may comprise a plurality of channels comprising a plurality of dimensions and properties. A fluidic device may comprise two channels with identical lengths. A fluidic device may comprise two channels that provide identical resistance. A fluidic device may comprise two identical channels.

A fluidic device may comprise a millichannel. A millichannel may have a width of between 100 and 200 mm. A millichannel may have a width of between 50 and 100 nm. A millichannel may have a width of between 20 and 50 nm. A millichannel may have a width of between 10 and 20 nm. A millichannel may have a width of between 1 and 10 nm. A fluidic device may comprise a microchannel. A microchannel may have a width of between 800 and 990 μm. A microchannel may have a width of between 600 and 800 μm. A microchannel may have a width of between 400 and 600 μm. A microchannel may have a width of between 200 and 400 μm. A microchannel may have a width of between 100 and 200 μm. A microchannel may have a width of between 50 and 100 μm. A microchannel may have a width of between 30 and 50 μm. A microchannel may have a width of between 20 and 30 μm. A microchannel may have a width of between 10 and 20 μm. A microchannel may have a width of between 5 and 10 μm. A microchannel may have a width of between 1 and 5μm. A fluidic device may comprise a nanochannel. A nanochannel may have a width of between 800 and 990 nm. A nanochannel may have a width of between 600 and 800 nm. A nanochannel may have a width of between 400 and 600 nm. A nanochannel may have a width of between 200 and 400 nm. A nanochannel may have a width of between 1 and 200 nm. A channel may have a comparable height and width. A channel may have a greater width than height, or a narrower width than height. A channel may have a width that is 1.1, 1.2, 1.3, 1.4, 1.5, 2, 3, 4, 5, 10, 20, 30, 40, 50, 100, 500, 1000 or more times its height. A channel may have a width that is 0.9, 0.8, 0.7, 0.6, 0.5, 0.25, 0.1, 0.05, 0.01, 0.005, 0.001 times its height. A channel may have a width that is less than 0.001 times its height. A channel may have non-uniform dimensions. A channel may have different dimensions at different points along its length. A channel may divide into 2 or more separate channels. A channel may be straight, or may have bends, curves, turns, angles, or other features of non-linear shapes. A channel may comprise a loop or multiple loops.

A fluidic device may comprise a resistance channel. A resistance channel may be a channel with slow flow rates relative to other channels within the fluidic device. A resistance channel may be a channel with low volumetric flow rates relative to other channels within the fluidic device. A resistance channel may provide greater resistance to sample flow relative to other channels in the fluidic device. A resistance channel may prevent or limit sample backflow. A resistance channel may prevent or limit cross-contamination between multiple samples within a device by limiting turbulence. A resistance channel may contribute to flow stability within a fluidic device. A resistance channel may limit disparities in flow rates between multiple portions of a fluidic device. A resistance channel may stabilize flow rates within a device, and minimize flow variation over time.

The flow of liquid in a fluidic device may be controlled with a plurality of microvalves. For example, the flow of liquid in this fluidic device may be controlled using up to four (4) microvalves, labelled in FIG. 3 as V1-V4. These valves can be electro-kinetic microvalves, pneumatic microvalves, vacuum microvalves, capillary microvalves, pinch microvalves, phase-change microvalves, burst microvalves.

The flow to and from the fluidic channel from each of P1-P4 is controlled by valves, labelled as V1-V4. The volume of liquids pumped into the ports can vary from nL to mL depending in the overall size of the device.

In device iteration 2.1, shows in FIG. 3, no amplification is needed. After addition of sample and pre-complexed programmable nuclease mix in P1 and P2, respectively, the reagents may be mixed in the serpentine channel, S1, which then leads to chamber C1 where the mixture may be incubated at the required temperature and time. The readout can be done simultaneously in C1, described in FIG. 4. Thermoregulation in C1 may be carried out using a thin-film planar heater manufactured, from e.g. Kapton, or other similar materials, and controlled by a proportional integral derivative (PID).

In device iteration 2.2, shown in FIG. 3, after addition of sample, amplification mix, and pre-complexed programmable nuclease mix in P1, P2 and P3, respectively, the reagents can be mixed in the serpentine channel, S1, which then leads to chamber C1 where the mixture is incubated at the required temperature and time needed to efficient amplification, as per the conditions of the method used. The readout may be done simultaneously in C1, described in FIG. 4. Thermoregulation may be achieved as previously described.

In device iteration 2.3, shown in FIG. 3, amplification and programmable nuclease reactions occur in separate chambers. The pre-complexed programmable nuclease mix is pumped into the amplified mixture from C1 using pump P3. The liquid flow is controlled by valve V3, and directed into serpentine mixer S2, and subsequently in chamber C2 for incubation the required temperature, for example at 37° C. for 90 minutes.

During the detection step (shown as step 4 in the workflow diagram of FIG. 1), the Cas-gRNA complex binds to its matching nucleic acid target from the amplified sample and is activated into a non-specific nuclease, which cleaves a nucleic acid-based reporter molecule to generate a signal readout. In the absence of a matching nucleic acid target, the Cas-gRNA complex does not cleave the nucleic acid-based reporter molecule. Real-time detection of the Cas reaction can be achieved by three methods: (1) fluorescence, (2) electrochemical detection, and (3) electrochemiluminescence. All three methods are described below and a schematic diagrams of these processes is shown in FIG. 4. Detection of the signal can be achieved by multiple methods, which can detect a signal that is calorimetric, potentiometric, amperometric, optical (e.g., fluorescent, colorometric, etc.), or piezo-electric, as non-limiting examples.

FIG. 4 shows schematic diagrams of a readout process that may be used in conjunction with a fluidic device (e.g., the fluidic device of FIG. 3), including (a) fluorescence readout and (b) electrochemical readout. The emitted fluorescence of cleaved reporter oligo nucleotides may be monitored using a fluorimeter positioned directly above the detection and incubation chamber. The fluorimeter may be a commercially available instrument, the optical sensor of a mobile phone or smart phone, or a custom-made optical array comprising of fluorescence excitation means, e.g. CO₂, other, laser and/or light emitting diodes (LEDs), and fluorescence detection means e.g. photodiode array, phototransistor, or others. A device may comprise a chamber comprising transparent or translucent materials that allow light to pass in and out of the chamber.

The fluorescence detection and excitation may be multiplexed, wherein, for example, fluorescence detection involves exciting and detecting more than one fluorophore in the incubation and detection chamber (C1 or C2). The fluorimeter itself may be multichannel, in which detecting and exciting light at different wavelengths, or more than one fluorimeter may be used in tandem, and their position above the incubation and detection chamber (C1 and C2) be modified by mechanical means, such as a motorized mechanism using micro or macro controllers and actuators (electric, electronic, and/or piezo-electric).

Two electrochemical detection variations are described herein, using integrated working, counter and reference electrodes in the incubation and detection chamber (C1 or C2):

Increase in signal. The progress of the cleavage reaction catalyzed by the programmable nuclease may be detected using a streptavidin-biotin coupled reaction. The top surface of the detection and incubation chamber may be functionalized with nucleic acid molecules (ssRNA, ssDNA or ssRNA/DNA hybrid molecules) conjugated with a biotin moiety. The bottom surface of the detection and incubation chamber operates as an electrode, comprising of working, reference, and counter areas, manufactured (or screen-printed) from carbon, graphene, silver, gold, platinum, boron-doped diamond, copper, bismuth, titanium, antimony, chromium, nickel, tin, aluminum, molybdenum, lead, tantalum, tungsten, steel, carbon steel, cobalt, indium tin oxide (ITO), ruthenium oxide, palladium, silver-coated copper, carbon nano-tubes, or other metals. The bottom surface of the detection and incubation chamber may be coated with streptavidin molecules. In the absence of any biotin molecules, the current measured by a connected electrochemical analyzer (commercial, or custom-made) is low. When the pre-complexed programmable nuclease mix with amplified target flows in the detection and incubation chamber, and is activated at a higher temperature, for example at 37° C., cleavage of the single-stranded nucleic acid (ssNA) linker releases biotin molecules that can diffuse onto the streptavidin-coated bottom surface of the detection and incubation chamber. Because of the interaction of biotin and streptavidin molecules, an increase in the current is read by a coupled electrochemical analyzer.

In some cases, reporter cleavage may increase the intensity of an electrochemical signal (e.g., a potentiometric signal from a square wave or cyclic voltammogram). Reporter cleavage may increase the diffusion constant of an electroactive moiety in the reporter, which can lead to an increase of an electrochemical signal. Thus, in some cases, electrochemical signal increase proportional to the degree of transcollateral reporter cleavage.

Some DETECTR experiments may be sensitive to small changes in cleaved reporter concentration, allowing low concentrations of target nucleic acid to be detected or distinguished. An electrochemical DETECTR assay (a DETECTR assay that utilizes electrochemical detection) may be capable to detecting less than 100 nM target nucleic acid. An electrochemical DETECTR assay may be capable to detecting less than 10 nM target nucleic acid. An electrochemical DETECTR assay may be capable to detecting less than 1 nM target nucleic acid. An electrochemical DETECTR assay may be capable to detecting less than 100 pM target nucleic acid. An electrochemical DETECTR assay may be capable to detecting less than 10 pM target nucleic acid. An electrochemical DETECTR assay may be capable to detecting less than 1 pM target nucleic acid. An electrochemical DETECTR assay may be capable to detecting less than 100 fM target nucleic acid. An electrochemical DETECTR assay may be capable to detecting less than 50 fM target nucleic acid. An electrochemical DETECTR assay may be capable to detecting less than 10 fM target nucleic acid. An electrochemical DETECTR assay may be capable to detecting less than 1 fM target nucleic acid. In some cases, an electrochemical detection may be more sensitive than fluorescence detection. In some cases, a DETECTR assay with electrochemical detection may have a lower detection limit than a DETECTR assay that utilizes fluorescence detection.

In some cases, an electrochemical DETECTR reaction may require low reporter concentrations. In some cases, an electrochemical DETECTR reaction may require low reporter concentrations. An electrochemical DETECTR reaction may require less than 10 μM reporter. An electrochemical DETECTR reaction may require less than 1 μM reporter. An electrochemical DETECTR reaction may require less than 100 nM reporter. An electrochemical DETECTR reaction may require less than 10 nM reporter. An electrochemical DETECTR reaction may require less than 1 nM reporter. An electrochemical DETECTR reaction may require less than 100 pM reporter. An electrochemical DETECTR reaction may require less than 10 pM reporter. An electrochemical DETECTR reaction may require less than 1 pM reporter.

Other types of signal amplification that use enrichment may also be used apart from biotin-streptavidin excitation. Non-limiting examples are: (1) glutathione, glutathione S-transferase, (2) maltose, maltose-binding protein, (3) chitin, chitin-binding protein.

Decrease in signal. The progress of the programmable nuclease cleavage reaction may be monitored by recording the decrease in the current produced by a ferrocene (Fc), or other electroactive mediator moieties, conjugated to the individual nucleotides of nucleic acid molecules (ssRNA, ssDNA or ssRNA/DNA hybrid molecules) immobilized on the bottom surface of the detection and incubation chamber. In the absence of the amplified target, the programmable nuclease complex remains inactive, and a high current caused by the electroactive moieties is recorded. When the programmable nuclease complex with guides flows in the detection and incubation chamber and is activated by the matching nucleic acid target at 37° C., the programmable nuclease complex non-specifically degrades the immobilized Fc-conjugated nucleic acid molecules. This cleavage reaction decreases the number of electroactive molecules and, thus, leads to a decrease in recorded current.

The electrochemical detection may also be multiplexed. This is achieved by the addition of one or more working electrodes in the incubation and detection chamber (C1 or C2). The electrodes can be plain, or modified, as described above for the single electrochemical detection method.

Electrochemiluminescence in a combined optical and electrochemical readout method. The optical signal may be produced by luminescence of a compound, such as tri-propyl amine (TPA) generated as an oxidation product of an electroactive product, such as ruthenium bipyridine,[Ru (py)3]2+.

A number of different programmable nuclease proteins may be multiplexed by: (1) separate fluidic paths (parallelization of channels), mixed with the same sample, for each of the proteins, or (2) switching to digital (two-phase) microfluidics, where each individual droplet contains a separate reaction mix. The droplets could be generated from single or double emulsions of water and oil. The emulsions are compatible with programmable nuclease reaction, and optically inert.

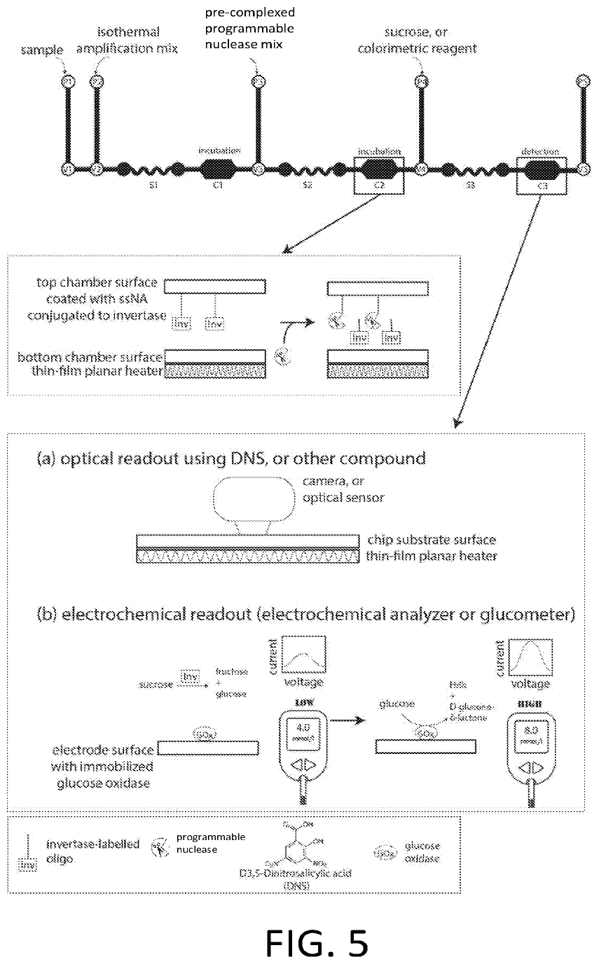

FIG. 5 shows an example fluidic device for coupled invertase/Cas reactions with colorimetric or electrochemical/glucometer readout. This diagram illustrates a fluidic device for miniaturizing a Cas reaction coupled with the enzyme invertase. Surface modification and readout processes are depicted in exploded view schemes at the bottom including (a) optical readout using DNS, or other compound and (b) electrochemical readout (electrochemical analyzer or glucometer). Described herein is the coupling of the Cas reaction with the enzyme invertase (EC 3.2.1.26), or sucrase or β-fructofuranosidase. This enzyme catalyzes the breakdown of sucrose to fructose and glucose.

The following methods may be used to couple the readout of the Cas reaction to invertase activity:

Colorimetry using a camera, standalone, or an integrated mobile phone optical sensor. The amount of fructose and glucose is linked to a colorimetric reaction. Two examples are: (a) 3,5-Dinitrosalicylic acid (DNS), and (b) formazan dye thiazolyl blue. The color change can be monitored using a CCD camera, or the image sensor of a mobile phone. For this method, we use a variation of the fluidic device described in FIG. 5. The modification is the use of a camera, instead of a fluorimeter above C3.

Amperometry using a conventional glucometer, or an electrochemical analyzer. A variation of the fluidic device described in FIG. 3 may be used, for example, the addition of one more incubation chamber C3. An additional step is added to the reaction scheme, which takes place in chamber C2. The top of the chamber surface is coated with single stranded nucleic acid that is conjugated to the enzyme invertase (Inv). The target-activated programmable nuclease complex cleaves the invertase enzyme from the oligo (ssRNA, ssDNA or ssRNA/DNA hybrid molecule), in C2, and invertase is then available to catalyze the hydrolysis of sucrose injected by pump P4, and controlled by valve V4. The mixture is mixed in serpentine mixer S3, and at chamber C3, the glucose produced may be detected colorimetrically, as previously described, electrochemically. The enzyme glucose oxidase is dried on the surface on C3, and catalyzes the oxidation of glucose to hydrogen peroxide and D-glucono-δ-lactone.

A number of different devices are compatible with detection of target nucleic acids using the methods and compositions disclosed herein. In some embodiments, the device is any of the microfluidic devices disclosed herein. In other embodiments, the device is a lateral flow test strip connected to a reaction chamber. In further embodiments, the lateral flow strip may be connected to a sample preparation device.

In some embodiments, the fluidic device may be a pneumatic device. The pneumatic device may comprise one or more sample chambers connected to one or more detection chambers by one or more pneumatic valves. Optionally, the pneumatic device may further comprise one or more amplification chamber between the one or more sample chambers and the one or more detection chambers. The one or more amplification chambers may be connected to the one or more sample chambers and the one or more detection chambers by one or more pneumatic valves. A pneumatic valve may be made from PDMS, or any other suitable material. A pneumatic valve may comprise a channel perpendicular to a microfluidic channel connecting the chambers and allowing fluid to pass between chambers when the valve is open. In some embodiments, the channel deflects downward upon application of air pressure through the channel perpendicular to the microfluidic channel.

In some embodiments, the fluidic device may be a sliding valve device. The sliding valve device may comprise a sliding layer with one or more channels and a fixed layer with one or more sample chambers and one or more detection chambers. Optionally, the fixed layer may further comprise one or more amplification chambers. In some embodiments, the sliding layer is the upper layer and the fixed layer is the lower layer. In other embodiments, the sliding layer is the lower layer and the fixed layer is the upper layer. In some embodiments, the upper layer is made of a plastic polymer comprising poly-methacrylate (PMMA), cyclic olefin polymer (COP), cyclic olefin copolymer (COC), polyethylene (PE), high-density polyethylene (HDPE), polypropylene (PP); a glass; or a silicon. In some embodiments, the lower layer is made of a plastic polymer comprising poly-methacrylate (PMMA), cyclic olefin polymer (COP), cyclic olefin copolymer (COC), polyethylene (PE), high-density polyethylene (HDPE), polypropylene (PP); a glass; or a silicon.The sliding valve device may further comprise one or more of a side channel with an opening aligned with an opening in the sample chamber, a side channel with an opening aligned with an opening in the amplification chamber, or a side channel with an opening aligned with the opening in the detection chamber. In some embodiments the side channels are connected to a mixing chamber to allow transfer of fluid between the chambers. In some embodiments, the sliding valve device comprises a pneumatic pump for mixing, aspirating, and dispensing fluid in the device.

Pneumatic Valve Device. A microfluidic device particularly well suited for carrying out the DETECTR reactions described herein is one comprising a pneumatic valve, also referred to as a “quake valve”. The pneumatic valve can be closed and opened by the flow of air from, for an example, an air manifold. The opening of the pneumatic valve can lead to a downward deflection of the channel comprising the pneumatic valve, which can subsequently deflect downwards and seal off a microfluidic channel beneath the channel comprising the pneumatic valve. This can lead to stoppage of fluid flow in the microfluidic channel. When the air manifold is turned off, the flow of air through the channel comprising the quake valve ceases and the microfluidic channel beneath the channel comprising the quake valve is “open”, and fluid can flow through. In some embodiments, the channel comprising the pneumatic valve may be above or below the microfluidic channel carrying the fluid of interest. In some embodiments, the channel comprising the pneumatic valve can be parallel or perpendicular to the microfluidic channel carrying the fluid of interest. Pneumatic valves can be made of a two hard thermoplastic layers sandwiching a soft silicone layer.

One example layout that is compatible with the compositions and methods disclosed herein is shown in FIG. 55 and FIG. 55. In some embodiments, the device comprises a sample chamber and a detection chamber, wherein the detection chamber is fluidically connected to the sample chamber by a pneumatic valve and wherein the detection chamber comprises any programmable nuclease of the present disclosure. Optionally, the device can also include an amplification chamber that is between the fluidic path from the sample chamber to the detection chamber, is connected to the sample chamber by a pneumatic valve, and is additionally connected to the detection chamber by a pneumatic valve. In some embodiments, the pneumatic valve is made of PDMS, or any other material for forming microfluidic valves. In some embodiments, the sample chamber has a port for inserting a sample. The sample can be inserted using a swab. The sample chamber can have a buffer for lysing the sample. The sample chamber can have a filter between the chamber and the fluidic channel to the amplification or detection chambers. The sample chamber may have an opening for insertion of a sample. A sample can be incubated in the sample chamber for from 30 seconds to 10 minutes. The air manifold may until this point be on, pushing air through the pneumatic valve and keeping the fluidic channel between the sample chamber and the amplification or detection chambers closed. At this stage, the air manifold can be turned off, such that no air is passing through the pneumatic valve, and allowing the microfluidic channel to open up and allow for fluid flow from the sample chamber to the next chamber (e.g., the amplification or detection chambers). In devices where there is an amplification chamber, the lysed sample flows from the sample chamber into the amplification chamber. Otherwise, the lysed sample flows from the sample chamber into the detection chamber. At this stage, the air manifold is turned back on, to push air through the pneumatic valve and seal the microfluidic channel. The amplification chamber holds various reagents for amplification and, optionally, reverse transcription of a target nucleic acid in the sample. These reagents may include forward and reverse primers, a deoxynucleotide triphosphate, a reverse transcriptase, a T7 promoter, a T7 polymerase, or any combination thereof. The sample is allowed to incubate in the amplification chamber for from 5 minutes to 40 minutes. The amplified and, optionally reverse transcribed, sample is moved into the detection chamber as described above: the air manifold is turned off, ceasing air flow through the pneumatic valve and opening the microfluidic channel. The detection chamber can include any programmable nuclease disclosed herein, a guide RNA with a portion reverse complementary to a portion of the target nucleic acid, and any reporter disclosed herein. In some embodiments, the detection chamber may comprise a plurality of guide RNAs. The plurality of guide RNAs may have the same sequence, or one or more of the plurality of guide RNAs may have different sequences. In some embodiments, the plurality of guide RNAs has a portion reverse complementary to a portion of a target nucleic acid different than a second RNA of the plurality of guide RNAs. The plurality of guide RNAs may comprise at least 5, at least 10, at least 15, at least 20, or at least 50 guide RNAs. Once the sample is moved into the detection chamber, the DETECTR reaction can be carried out for 1 minute to 20 minutes. Upon hybridization of the guide RNA to the target nucleic acid, the programmable nuclease is activated and begins to collaterally cleave the reporter, which as described elsewhere in this disclosure has a nucleic acid and one or more molecules that enable detection of cleavage. The detection chamber can interface with a device for reading out for the signal. For example, in the case of a colorimetric or fluorescence signal generated upon cleavage, the detection chamber may be coupled to a spectrophotometer or fluorescence reader. In the case where an electrochemical signal is generated, the detection chamber may have one to 10 metal leads connected to a readout device (e.g., a glucometer), as shown in FIG. 60. FIG. 59 shows a schematic of the top layer of a cartridge of a pneumatic valve device of the present disclosure, highlighting suitable dimensions. The schematic shows one cartridge that is 2 inches by 1.5 inches. FIG. 60 shows a schematic of a modified top layer of a cartridge of a pneumatic valve device of the present disclosure adapted for electrochemical dimension. In this schematic, three lines are shown in the detection chambers (4 chambers at the very right). These three lines represent wiring (or “metal leads”), which is co-molded, 3D-printed, or manually assembled in the disposable cartridge to form a three-electrode system. Electrodes are termed as working, counter, and reference. Electrodes can also be screen printed on the cartridges. Metals used can be carbon, gold, platinum, or silver. A major advantage of the pneumatic valve device is that the pneumatic valves connecting the various chambers of the device prevent backflow from chamber to chamber, which reduces contamination. Prevention of backflow and preventing sample contamination is especially important for the applications described herein. Sample contamination can result in false positives or can generally confound the limit of detection for a target nucleic acid. As another example, the pneumatic valves disclosed herein are particularly advantageous for devices and methods for multiplex detection. In multiplexed assays, where two or more target nucleic acids are assayed for, it is particularly important that backflow and contamination is avoided. Backflow between chambers in a multiplexed assay can lead to cross-contamination of different guide nucleic acids or different programmable nuclease and can result in false results. Thus, the pneumatic valve device, which is designed to minimize or entirely avoid backflow, is particularly superior, in comparison to other device layouts, for carrying out the detection methods disclosed herein.

FIG. 55 shows a quake valve pneumatic pump layout for a DETECTR assay. FIG. 55A shows a schematic of a pneumatic valve device. A pipette pump aspirates and dispenses samples. An air manifold is connected to a pneumatic pump to open and close the normally closed valve. The pneumatic device moves fluid from one position to the next. The pneumatic design has reduced channel cross talk compared to other device designs. FIG. 55B shows a schematic of a cartridge for use in the pneumatic valve device shown in FIG. 55A. The valve configuration is shown. The normally closed valves (one such valve is indicated by an arrow) comprise an elastomeric seal on top of the channel to isolate each chamber from the rest of the system when the chamber is not in use. The pneumatic pump uses air to open and close the valve as needed to move fluid to the necessary chambers within the cartridge. FIG. 56 shows a valve circuitry layout for the pneumatic valve device shown in FIG. 55A. A sample is placed in the sample well while all valves are closed, as shown at (i.). The sample is lysed in the sample well. The lysed sample is moved from the sample chamber to a second chamber by opening the first quake valve, as shown at (ii.), and aspirating the sample using the pipette pump. The sample is then moved to the first amplification chamber by closing the first quake valve and opening a second quake valve, as shown at (iii.) where it is mixed with the amplification mixture. After the sample is mixed with the amplification mixture, it is moved to a subsequent chamber by closing the second quake valve and opening a third quake valve, as shown at (iv). The sample is moved to the DETECTR chamber by closing the third quake valve and opening a fourth quake valve, as shown at (v). The sample can be moved through a different series of chambers by opening and closing a different series of quake valves, as shown at (vi). Actuation of individual valves in the desired chamber series prevents cross contamination between channels. In some embodiments the sliding valve device has a surface area of 5 cm by 5 cm, 5 by 6 cm, 6 by 7 cm, 7 by 8 cm, 8 by 9 cm, 9 by 10 cm, 10 by 11 cm, 11 by 12 cm, 6 by 9 cm, 7 by 10 cm, 8 by 11 cm, 9 by 12 cm, 10 by 13 cm, 11 by 14 cm, 12 by 11 cm, about 30 sq cm, about 35 sq cm, about 40 sq cm, about 45 sq cm, about 50 sq cm, about 55 sq cm, about 60 sq cm, about 65 sq cm, about 70 sq cm, about 75 sq cm, about 25 sq cm, about 20 sq cm, about 15 sq cm, about 10 sq cm, about 5 sq cm, from 1 to 100 sq cm, from 5 to 10 sq cm, from 10 to 15 sq cm, from 15 to 20 sq cm, from 20 to 25 sq cm, from 25 to 30 sq cm, from 30 to 35 sq cm, from 35 to 40 sq cm, from 40 to 45 sq cm, from 45 to 50 sq cm, from 5 to 90 sq cm, from 10 to 0 sq cm, from 15 to 5 sq cm, from 20 to 10 sq cm, or from 25 to 15 sq cm.

Sliding Valve Device. A microfluidic device particularly well suited for carrying out the DETECTR reactions described herein is a sliding valve device. The sliding valve device can have a sliding layer and a fixed layer. The sliding layer may be on top and the fixed layer may be on bottom. Alternatively, the sliding layer may be on bottom and the fixed layer may be on top. In some embodiments, the sliding valve has a channel. The channel can have an opening at one end that interacts with an opening in a chamber and the channel can also have an opening at the other end that interacts with an opening in a side channel. In some embodiments, the sliding layer has more than one opening. In some embodiments, the fixed layer comprises a sample chamber, an amplification chamber, and a detection chamber. The sample chamber, the amplification chamber, and the detection layer can all have an opening at the bottom of the chambers. For example, the sample chamber may have an opening for insertion of a sample. When the opening in a chamber is aligned with the opening in a channel, fluid can flow from the chamber into the channel. Further, when the opening in the channel is subsequently aligned with an opening in a side channel, fluid can flow from the channel into the side channel. The side channel can be further fluidically connected to a mixing chamber, or a port in which an instrument (e.g., a pipette pump) for mixing fluid is inserted. Alignment of openings can be enabled by physically moving or automatically actuating the sliding layer to slide along the length of the fixed layer. In some embodiment, the above described pneumatic valves can be added at any position to the sliding valve device in order to control the flow of fluid from one chamber into the next. The sliding valve device can also have multiple layers. For example, the sliding valve can have 2, 3, 4, 5, 6, 7, 8, 9, 10, or more layers.

FIG. 46 shows a layout for a DETECTR assay. Shown at top is a pneumatic pump, which interfaces with the cartridge. Shown at middle is a top down view of the cartridge showing a top layer with reservoirs. Shown at bottom is a sliding valve containing the sample and arrows pointing to the lysis chamber at left, following by amplification chambers to the right, and DETECT chambers further to the right. FIG. 57 shows a schematic of a sliding valve device. The offset pitch of the channels allows aspirating and dispensing into each well separately and helps to mitigate cross talk between the amplification chambers and corresponding chambers. FIG. 58 shows a diagram of sample movement through the sliding valve device shown in FIG. 57. In the initial closed position (i.), the sample is loaded into the sample well and lysed. The sliding valve is then actuated by the instrument, and samples are loaded into each of the channels using the pipette pump, which dispenses the appropriate volume into the channel (ii.). The sample is delivered to the amplification chambers by actuating the sliding valve and mixed with the pipette pump (iii.). Samples from the amplification chamber are aspirated into each channel (iv.) and then dispensed and mixed into each DETECTR chamber (v.) by actuating the sliding valve and pipette pump. In some embodiments the sliding valve device has a surface area of 5 cm by 8 cm, 5 by 6 cm, 6 by 7 cm, 7 by 8 cm, 8 by 9 cm, 9 by 10 cm, 10 by 11 cm, 11 by 12 cm, 6 by 9 cm, 7 by 10 cm, 8 by 11 cm, 9 by 12 cm, 10 by 13 cm, 11 by 14 cm, 12 by 11 cm, about 30 sq cm, about 35 sq cm, about 40 sq cm, about 45 sq cm, about 50 sq cm, about 55 sq cm, about 60 sq cm, about 65 sq cm, about 70 sq cm, about 75 sq cm, about 25 sq cm, about 20 sq cm, about 15 sq cm, about 10 sq cm, about 5 sq cm, from 1 to 100 sq cm, from 5 to 10 sq cm, from 10 to 15 sq cm, from 15 to 20 sq cm, from 20 to 25 sq cm, from 25 to 30 sq cm, from 30 to 35 sq cm, from 35 to 40 sq cm, from 40 to 45 sq cm, from 45 to 50 sq cm, from 5 to 90 sq cm, from 10 to 0 sq cm, from 15 to 5 sq cm, from 20 to 10 sq cm, or from 25 to 15 sq cm.

Lateral Flow Devices. In some embodiments, a device of the present disclosure comprises a chamber and a lateral flow strip. FIG. 32-FIG. 33 shows a particularly advantageous layout for the lateral flow strip and a corresponding suitable reporter. FIG. 32 shows a modified Cas reporter comprising a DNA linker to biotin-dT (shown as a pink hexagon) bound to a FAM molecule (shown as a green start). FIG. 33 shows the layout of Milenia HybridDetect strips with the modified Cas reporter. This particular layout improves the test result by generating higher signal in the case of a positive result, while also minimizing false positives. In this assay layout, the reporter comprises a biotin and a fluorophore attached at one of a nucleic acid. The nucleic acid can be conjugated directly to the biotin molecule and then the fluorophore or directly to the fluorophore and then to the biotin. Other affinity molecules, including those described herein can be used instead of biotin. Any of the fluorophores disclosed herein can also be used in the reporter. The reporter can be suspended in solution or immobilized on the surface of the Cas chamber. Alternatively, the reporter can be immobilized on beads, such as magnetic beads, in the reaction chamber where they are held in position by a magnet placed below the chamber. When the reporter is cleaved by an activated programmable nuclease, the cleaved biotin-fluorophore accumulates at the first line, which comprises a streptavidin (or another capture molecule). Gold nanoparticles, which are on the sample pad and flown onto the strip using a chase buffer, are coated with an anti-fluorophore antibody allowing binding and accumulation of the gold nanoparticle at the first line. The nanoparticles additionally accumulate at a second line which is coated with an antibody (e.g., anti-rabbit) against the antibody coated on the gold nanoparticles (e.g., rabbit, anti-FAM). In the case of a negative result, the reporter is not cleaved and does not flow on the lateral flow strip. Thus, the nanoparticles only bind and accumulate at the second line Multiplexing on the lateral flow strip can be performed by having two reporters (e.g., a biotin-FAM reporter and a biotin-DIG reporter). Anti-FAM and anti-DIG antibodies are coated onto the lateral flow strip at two different regions. Anti-biotin antibodies are coated on gold nanoparticles. Fluorophores are conjugated directly to the affinity molecules (e.g., biotin) by first generating a biotin-dNTP following from the nucleic acids of the reporter and then conjugating the fluorophore. In some embodiments, the lateral flow strip comprises multiple layers.

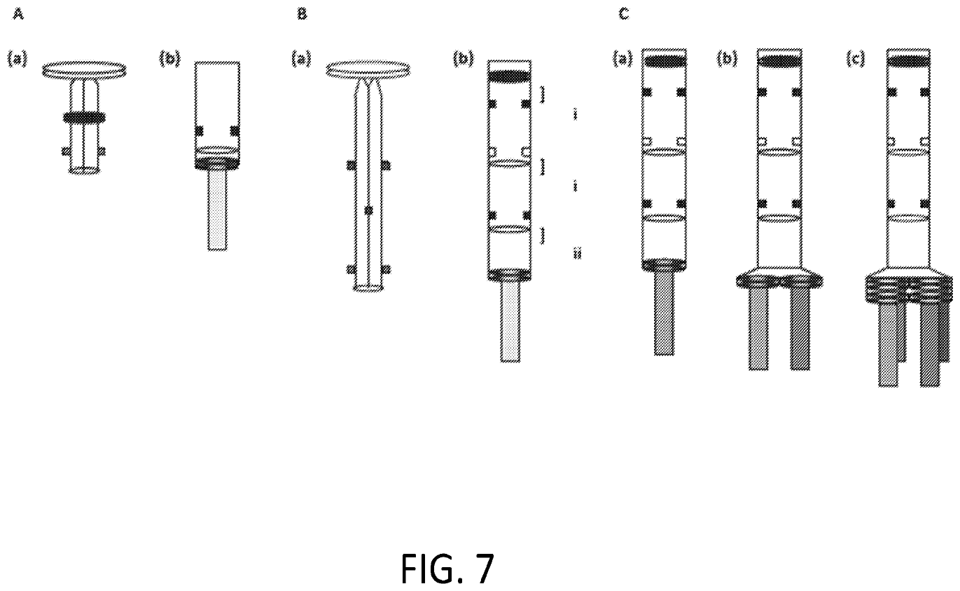

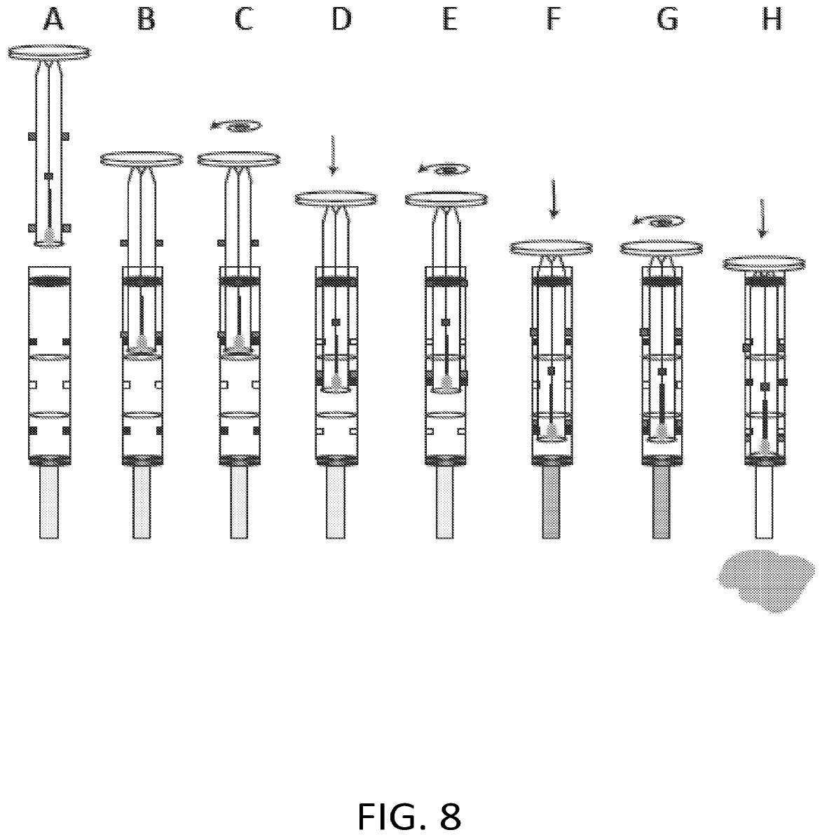

In some embodiments, the above lateral flow strip can be additionally interfaced with a sample preparation device, as shown in FIG. 7 and FIG. 8. FIG. 7 shows individual parts of sample preparation devices of the present disclosure. Part A of the figure shows a single chamber sample extraction device: (a) the insert holds the sample collection device and regulates the step between sample extraction and dispensing the sample into another reaction or detection device, (b) the single chamber contains extraction buffer. Part B of the figure shows filling the dispensing chamber with material that further purifies the nucleic acid as it is dispensed is an option: (a) the insert holds the sample collection device and regulates the “stages” of sample extraction and nucleic acid amplification. Each set of notches (red, blue and green) are offset 90° from the preceding set, (b) the reaction module contains multiple chambers separated by substrates that allow for independent reactions to occur. (e.g., i. a nucleic acid separation chamber, ii. a nucleic acid amplification chamber And iii. a DETECTR reaction chamber or dispensing chamber). Each chamber has notches (black) that prevent the insert from progressing into the next chamber without a deliberate 90° turn. The first two chambers may be separated by material that removes inhibitors between the extraction and amplification reactions. Part C shows options for the reaction/dispensing chamber: (a) a single dispensing chamber may release only extracted sample or extraction/amplification or extraction/amplification/DETECTR reactions, (b) a duel dispensing chamber may release extraction/multiplex amplification products, and (c) a quadruple dispensing chamber would allow for multiplexing amplification and single DETECTR or four single amplification reactions. FIG. 8 shows a sample work flow using a sample processing device. The sample collection device is attached to the insert portion of the sample processing device (A). The insert is placed into the device chamber and pressed until the first stop (lower tabs on top portion meet upper tabs on bottom portion) (B). This step allows the sample to come into contact with the nucleic acid extraction reagents. After the appropriate amount of time, the insert is turned 90° (C.) and depressed (D) to the next set of notches. These actions transfer the sample into the amplification chamber. The sample collection device is no longer in contact with the sample or amplification products. After the appropriate incubation, the insert is rotated 90° (E) and depressed (F) to the next set of notches. These actions release the sample into the DETECTR (green reaction). The insert is again turned 90° (G) and depressed (H) to dispense the reaction.

Resistance Channel Devices. In some embodiments, a device of the present disclosure may resistance channels, sample metering channels, valves for fluid flow or any combination thereof. FIG. 126A, FIG. 126B, FIG. 127A, FIG. 127B, FIG. 128A, FIG. 128B, FIG. 128C, FIG. 128D, FIG. 129A, FIG. 129B, FIG. 129C, and FIG. 129D show examples of said microfluidic cartridges for use in a DETECTR reaction. In some embodiments, a cartridge may comprise an amplification chamber, a valve fluidically connected to the amplification chamber, a detection reaction chamber fluidically connected to the valve, and a detection reagent reservoir fluidically connected to the detection chamber, as shown in FIG. 130A. In some embodiments, a device may further comprise a luer slip adapter, as shown in FIG. 131C. A leur slip adaptor may be used to adapt to a leur lock syringe for sample or reagent delivery into the device. One or more elements (e.g., chambers, channels, valves, or pumps) of a microfluidic device may be fluidically connected to one or more other elements of the microfluidic device. A first element may be fluidically connected to a second element such that fluid may flow between the first element and the second element. A first element may be fluidically connected to a second element through a third element such that fluid may flow from the first element to the second element by passing through the third element. For example, a detection reagent chamber may be fluidically connected to a detection chamber through a resistance channel, as shown in FIG. 130A.

A chamber of the device (e.g., the amplification chamber, the detection chamber, or the detection reagent reservoir) may be fluidically connected to one or more additional chambers by one or more channels. In some embodiments, a channel may be a resistance channel configured to regulate the flow of fluid between a first chamber and a second chamber. A resistance channel may form a non-linear path between the first chamber and the second chamber. It may include features to restrict or confound flow, such as bends, turns, fins, chevrons, herringbones or other microstructures. A resistance channel may have reduced backflow compared to a linear channel of comparable length and width. A resistance channel may function by requiring an increased pressure to pass fluid through the channel compared to a linear channel of comparable length and width. In some embodiments, a resistance channel may result in decreased cross-contamination between two chambers connected by the resistance channel as compared to the cross-contamination between two chambers connected by a linear channel of comparable length and width. A resistance channel may have an angular path, for example as illustrated FIG. 128A, FIG. 128B, FIG. 129C and FIG. 129D. An angular path may comprise one or more angles in the direction of flow of a fluid passing through the channel. In some embodiments, an angular path may comprise a right angle. In some embodiments, an angular path may comprise an angle of about 90°. In some embodiments, an angular path may comprise at least one angle between about 45° and about 135°. In some embodiments, an angular path may comprise at least one angle between about 80° and about 100°. In some embodiments, an angular path may comprise at least one angle between about 85° and about 95°. A resistance channel may have a circuitous or serpentine path, for example as illustrated in FIG. 128C, FIG. 128D, FIG. 129A, and FIG. 129B. A circuitous or serpentine path may comprise one or more bends in the direction of flow of a fluid passing through the channel. In some embodiments, a circuitous or serpentine path may comprise a bend of about 90°. In some embodiments, a circuitous or serpentine path may comprise at least one bend between about 45° and about 135°. In some embodiments, a circuitous or serpentine path may comprise at least one bend between about 80° and about 100°. In some embodiments, a circuitous or serpentine path may comprise at least one bend between about 85° and about 95°. In some embodiments, a resistance channel may be substantially contained within a plane (e.g., the resistance channel may be angular, circuitous, or serpentine in two-dimensions). A two-dimensional resistance channel may be positioned substantially within a single layer of a microfluidic device of the present disclosure. In some embodiments, a resistance channel may be a three-dimensional resistance channel (e.g., the resistance channel may be angular, circuitous, or serpentine in x, y, and z dimensions of a microfluidic device). In some embodiments, a sample input of a resistance channel may be in the same plane (e.g., at the same level in a z direction) as the resistance channel, a chamber connected to the resistance channel, or both. In some embodiments, a sample input of a resistance channel may be in a different plan (e.g., on a different level in a z direction) as the resistance channel, a chamber connected to the resistance channel, or both. Examples of resistance channels are shown in FIG. 133. In some embodiments a resistance channel may have a width of about 300 μm. In some embodiments a resistance channel may have a width of from about 10 μm to about 100 μm, from about 50 μm to about 100 μm, from about 100 μm to about 200 μm, from about 100 μm to about 300 μm, from about 100 μm to about 400 μm, from about 100 μm to about 500 μm, from about 200 μm to about 300 μm, from about 200 μm to about 400 μm, from about 200 μm to about 500 μm, from about 200 μm to about 600 μm, from about 200 μm to about 700 μm, from about 200 μm to about 800 μm, from about 200 μm to about 900 μm, or from about 200 μm to about 1000 μm.

In some embodiments, a channel may be a sample metering channel. A sample metering channel may form a path between a first chamber and a second chamber and have a channel volume configured to hold a set volume of a fluid to meter the volume of fluid transferred from the first chamber to the second chamber. A sample metering path may form a path between a first chamber and a second chamber and have a channel volume configured to allow to flow from the first channel to the second channel at a desired rate. Metering can also be affected by positive or negative pressure applied to an auxiliary chamber acting as a liquid reagent storage reservoir. This can also be done by storing air in a blister pack for low-cost applications. Examples of sample metering channels are shown in FIG. 133. In some embodiments, a sample input of a sample metering channel may be in the same plane (e.g., at the same level in a z direction) as the sample metering channel, a chamber connected to the sample metering channel, or both. In some embodiments, a sample input of a sample metering channel may be in a different plan (e.g., on a different level in a z direction) as the sample metering channel, a chamber connected to the sample metering channel, or both. The length, width, volume, or combination thereof of a sample metering channel may be designed to pass a desired volume of fluid from a first chamber to a second chamber. The length, width, volume, or combination thereof of a sample metering channel may be designed to pass fluid from a first chamber to a second chamber at a desired rate. In some embodiments, a sample metering channel may have a width of about 300 μm. In some embodiments a sample metering channel may have a width of from about 10 μm to about 100 μm, from about 50 μm to about 100 μm, from about 100 μm to about 200 μm, from about 100 μm to about 300 μm, from about 100 μm to about 400 μm, from about 100 μm to about 500 μm, from about 200 μm to about 300 μm, from about 200 μm to about 400 μm, from about 200 μm to about 500 μm, from about 200 μm to about 600 μm, from about 200 μm to about 700 μm, from about 200 μm to about 800 μm, from about 200 μm to about 900 μm, or from about 200 μm to about 1000 μm. In some embodiments, a first chamber may be connected to a second chamber by a channel comprising a resistance channel and a sample metering channel.

A schematic example of a resistance channel is shown in FIG. 133. The valve seat may have a reduced height of about 142 μm and the valve has a dead volume of about 2 μL. The valve may be positioned on a different plane than the sample metering channel to minimize the seat height and the dead volume and to improve sealing. The DETECTR sample metering inlet may be positioned on a different level than the sample metering channel so that the sample enters the channel at a different height to prevent amplified sample entry or backflow. The sample metering channel may have an increased height of about 784 μm to accommodate 5 μL of metered sample with a footprint of about 0.784 mm×0.75 mm×8.25 mm, as compared to a channel with a height of 142 μm and a footprint of about 0.142 mm×0.75 mm×46 mm. The DETECTR sample detection well inlet may be positioned on a different level than the mixing well so that the DETECTR sample enters the detection well at a different level to reduce the cross sectional area and reduce backflow.

A microfluidic device may comprise one or more reagent ports configured to receive a reagent into the device (e.g., into a chamber of the device). A reagent port may comprise an opening in the wall of a chamber. A reagent port may comprise an opening in the wall of a channel or the end of a channel. A reagent port configured to receive a sample may be a sample inlet port. A reagent (e.g., a buffer, a solution, or a sample) may be introduced into the microfluidic device through a reagent port. The reagent may be introduced manually by a user (e.g., a human user), or the reagent may be introduced automatically by a machine (e.g., by a detection manifold).

A variety of chamber shapes may be utilized in the cartridges of the present disclosure. A chamber may be circular, for example the amplification chambers, detection chambers, and detection reagent reservoirs shown in FIG. 128A and FIG. 128C. A chamber may be elongated, for example the amplification chambers and detection reagent reservoirs shown in FIG. 128B, FIG. 128D, FIG. 129A, FIG. 129B, FIG. 129C, and FIG. 129D.

A valve may be configured to prevent, regulate, or allow fluid flow from a first chamber to one or more additional chambers. In some embodiments, a valve may rotate from a first position to a second position to prevent, allow, or alter a fluid flow path. In some embodiments, a valve may slide from a first position to a second position to prevent, allow, or alter a fluid flow path. In some embodiments, a valve may open or close based on pressure applied to the valve. In some embodiments, a valve may be an elastomeric valve. The valve can be active (mechanical, non-mechanical, or externally actuated) or passive (mechanical or non-mechanical). A valve may be a push-pull/solenoid actuated valve. A valve may be controlled electronically. For example, a valve may be controlled using a solenoid. In some embodiments, a valve may be controlled manually. Other mechanisms of control may be: magnetic, electric, piezoelectric, thermal, bistable, electrochemical, phase change, rheological, pneumatic, check valving or capillarity. In some embodiment, a valve may be disposable. For example, a valve may be removed from a microfluidic device and replaced with a new valve to prevent contamination when reusing a microfluidic device. In some embodiments, a valve may be covered by a valve cap or elastomeric plug.

The cartridge may be configured to connect to a first pump to pump fluid from the amplification chamber to the detection chamber and to a second pump to pump fluid from the detection reagent reservoir to the detection chamber. A variety of pumps known in the art are functional to move fluid from a first chamber to a second chamber and may be used with a cartridge of the present disclosure. In some embodiments, a cartridge may be used with a peristaltic pump, a pneumatic pump, a hydraulic pump, or a syringe pump.

An example of a microfluidic cartridge is shown in FIG. 127A and FIG. 127B. As shown in FIG. 127A, the cartridge may contain an amplification chamber and sample inlet well capable of storing about 45 μL of aqueous reaction mix to which a user adds about 5 μL of sample. The amplification chamber may be sealed. A pump air inlet interfaces the cartridge to an external low-volume low-power pump for solution control. The on-board cartridge valve may be configured to contain amplification mixture during the heating step and during pressure build-up. The cartridge ma contain an amplification mix splitter to split the incoming amplification reaction mix and allows a pump to dispense about 5 μL directly to the detection chambers. Dual detection chambers can be vented with hydrophobic PTFE vent to allow solution entry, have a clear top for imaging and detection, and may be heated to 37° C. for 10 minutes during a reaction. In some embodiments, a detection chamber may be sized such that an amplified sample mixture fills the detection chamber when combined with the detection reagents from the detection reagent storage chamber. DETECTR reaction mix storage wells, also referred to as a detection reagent storage chambers, can store about 100 μL, of aqueous DETECTR mix on-board the cartridge. The pump air inlet interfaces the cartridge to an external low-volume low-power pump for solution control. As shown in FIG. 127B, the cartridge may contain a cartridge air supply valves, and entries sit above aqueous reagent to prevent overspill. Passive reagent fill stops form a torturous path and have hydrostatic head to passively prevent aqueous solution flow into cartridge after filling. The on-board elastomeric valve prevents forward flow under pressure build-up from the reaction mixture heated to 65° C. and is actuated by a low-cost, small-footprint linear actuator.

In some embodiments, a device may comprise a multi-layered, laminated cartridge patterned with laser embossing, and hardware with integrated electronics, optics and mechanics, as shown in FIG. 130B. A multi-layered device may be manufactured by two-dimensional lamination, as shown in FIG. 131B (left). In some embodiments, a device may be injection molded. An injection molded device may be laminated to seal the device, as shown in FIG. 131B (right). Injection molding may be used for high volume production of a microfluidic device of the present disclosure.

Detection Manifolds. A detection manifold may be used to perform and detect a DETECTR assay of the present disclosure in a device of the present disclosure. A detection manifold may also be referred to herein as a cartridge manifold or a heating manifold. A detection manifold may be configured to facilitate or detect a DETECTR reaction performed in a microfluidic device of the present disclosure. In some embodiments, a detection manifold may comprise one or more heating zones to heat one or more regions of a microfluidic device. In some embodiments, a detection manifold may comprise a first heating zone to heat a first region of a microfluidic device in which an amplification reaction is performed. For example, the first heater may heat the first region of the microfluidic device to about 60° C. In some embodiments, a detection manifold may comprise a second heating zone to heat a second region of a microfluidic device in which a detection reaction is performed. For example, the second heater may heat the second region of the microfluidic device to about 37° C. In some embodiments, a detection manifold may comprise a third heating zone to heat a third region of a microfluidic device in which a lysis reaction is performed. For example, the third heater may heat the third region of the microfluidic device to about 95° C. An example of a detection manifold comprising two insulated heating zones for use with a microfluidic cartridge is shown in FIG. 131A. In some embodiments, a detection manifold may comprise a heating zone configured to heat a lysis region of a microfluidic device of the presence disclosure. An example of a detection manifold comprising a lysis heating zone, an amplification heating zone, and a detection heating zone is shown in FIG. 132A and FIG. 132B. The detection manifold may be configured to be compatible with a microfluidic device comprising a lysis chamber, an amplification chamber, and a detection chamber.

In some embodiments, a detection manifold may comprise an illumination source configured to illuminate a detection chamber of a microfluidic device. The illumination source may be configured to emit a narrow spectrum illumination (e.g., an LED) or the illumination may be configured to emit a broad-spectrum illumination (e.g., an arc lamp). The detection manifold may further comprise one or more filters or gratings to filter for a desired illumination wavelength. In some embodiments, the illumination source may be configured to illuminate a detection chamber (e.g., a chamber comprising a DETECTR reaction) through a top surface of a microfluidic device. In some embodiments, the illumination source may be configured to illuminate a detection chamber through a side surface of a microfluidic device. In some embodiments, the illumination source may be configured to illuminate a detection chamber through a bottom surface of a microfluidic device. In some embodiments, the detection manifold may comprise a sensor for detecting a signal produced by a DETECTR reaction. The signal may be a fluorescent signal. For example, the detection manifold may comprise a camera (e.g., charge-coupled device (CCD), complementary metal-oxide-semiconductor (CMOS)) or a photodiode. A schematic example of a detection manifold is shown in FIG. 136A and FIG. 136B. An example of a detection illuminated in a detection manifold is shown in FIG. 137A.

A detection manifold may comprise electronics configured to control one or more of a temperature, a pump, a valve, an illumination source, or a sensor. In some embodiments, the electronics may be controlled autonomously using a program. For example, the electronics may be autonomously controlled to implement a workflow of the present disclosure (e.g., the workflow provided in FIG. 134). A schematic example of an electronic layout is provided in FIG. 135. The electronics may control one or more heaters using one or more of a power control, a temperature feedback, or a PID loop. One or more of a pump, a valve (e.g., a solenoid-controlled valve), or an LED (e.g., a blue LED) may be controlled by one or more of a power converter (e.g., a 3V, 12V, or 9V power converter) or a power relay board. A logic board may be used to control one or more elements of the detection manifold. A detection manifold may comprise one or more indicator lights to indicate a status of one or more elements (e.g., an LED, a heater, a pump, or a valve). The devices described in this section may be combined with any other features disclosed herein (e.g., pneumatic valves, components that operate via use of sliding valves, or any other general feature of devices disclosed herein).

General Features of Devices. In some embodiments, a device of the present disclosure can hold 2 or more amplification chambers. In some embodiments, a device of the present disclosure can hold 10 or more detection chambers. In some embodiments, a device of the present disclosure comprises a single chamber in which sample lysis, target nucleic acid amplification, reverse transcription, and detection are all carried out. In some cases, different buffers are present in the different chambers. In some embodiments, all the chambers of a device of the present disclosure have the same buffer. In some embodiments, the sample chamber comprises the lysis buffer and all of the materials in the amplification and detection chambers are lyophilized or vitrified. In some embodiments, the sample chamber includes any buffer for lysing a sample disclosed herein. The amplification chamber can include any buffer disclosed herein compatible with amplification and/or reverse transcription of target nucleic acids. The detection chamber can include any DETECTR or CRISPR buffer (e.g., an MBuffer) disclosed herein or otherwise capable of allowing DETECTR reactions to be carried out. In this case, once sample lysing has occurred, volume is moved from the sample chamber to the other chambers in an amount enough to rehydrate the materials in the other chambers. In some embodiments, the device further comprises a pipette pump at one end for aspirating, mixing, and dispensing liquids. In some embodiments, an automated instrument is used to control aspirating, mixing, and dispensing liquids. In some embodiments, no other instrument is needed for the fluids in the device to move from chamber to chamber or for sample mixing to occur. A device of the present disclosure may be made of any suitable thermoplastic, such as COC, polymer COP, teflon, or another thermoplastic material. Alternatively, the device may be made of glass. In some embodiments, the detection chamber may include beads, such as nanoparticles (e.g., a gold nanoparticle). In some embodiments, the reporters are immobilized on the beads. In some embodiments, after cleavage from the bead, the liberated reporters flow into a secondary detection chamber, where detection of a generated signal occurs by any one of the instruments disclosed herein. In some embodiments, the detection chamber is shallow, but has a large surface area that is optimized for optical detection. A device of the present disclosure may also be coupled to a thermoregulator. For example, the device may be on top of or adjacent to a planar heater that can heat the device up to high temperatures. Alternatively, a metal rod conducting heat is inserted inside the device and presses upon a soft polymer. The heat is transferred to the sample by dissipating through the polymer and into the sample. This allows for sample heating with direct contact between the metal rod and the sample. In some embodiments, in addition to or in place of a buffer for lysing a sample, the sample chamber may include an ultrasonicator for sample lysis. A swab carrying the sample may be inserted directly into the sample chamber. Commonly, a buccal swab may be used, which can carry blood, urine, or a saliva sample. A filter may be included in any of the chambers in the devices disclosed herein to filter the sample prior to carrying it to the next step of the method. Any of the devices disclosed herein can be couple to an additional sample preparation module for further manipulation of the sample before the various steps of the DETECTR reaction. In some embodiments the reporter can be in solution in the detection chamber. In other embodiments, the reporter can be immobilized directly on the surface of the detection chamber. The surface can be the top or the bottom of the chamber. In still other embodiments, the reporter can be immobilized to the surface of a bead. In the case of a bead, after cleavage, the detectable signal may be washed into a subsequent chamber while the bead remains trapped—thus allowing for separation of the detectable signal from the bead. Alternatively, cleavage of the reporter off of the surface of the bead is enough to generate a strong enough detectable signal to be measured. By sequestering or immobilizing the above described reporters, the stability of the reporters in the devices disclosed herein carrying out DETECTR reactions may be improved. Any of the above devices can be compatible for colorimetric, fluorescence, amperometric, potentiometric, or another electrochemical signal. In some embodiments, the colorimetric, fluorescence, amperometric, potentiometric, or another electrochemical sign may be detected using a measurement device connected to the detection chamber (e.g., a fluorescence measurement device, a spectrophotometer, or an oscilloscope).

In some embodiments, signals themselves can be amplified, for example via use of an enzyme such as horse radish peroxidase (HRP). In some embodiments, biotin and avidin reactions, which bind at a 4:1 ratio can be used to immobilize multiple enzymes or secondary signal molecules (e.g., 4 enzymes of secondary signal molecules, each on a biotin) to a single protein (e.g., avidin). In some embodiments, an electrochemical signal may be produced by an electrochemical molecule (e.g., biotin, ferrocene, digoxigenin, or invertase). In some embodiments, the above devices could be couple with an additional concentration step. For example, silica membranes may be used to capture nucleic acids off a column and directly apply the Cas reaction mixture on top of said filter. In some embodiments, the sample chamber of any one of the devices disclosed herein can hold from 20 ul to 1000 ul of volume. In some embodiments, the sample chamber holds from 20 to 500, from 40 to 400, from 30 to 300, from 20 to 200 or from 10 to 100 ul of volume. In preferred embodiments, the sample chamber holds 200 ul of volume. The amplification and detection chambers can hold a lower volume than the sample chamber. For example, the amplification and detection chambers may hold from 1 to 50, 10 to 40, 20 to 30, 10 to 40, 5 to 35, 40 to 50, or 1 to 30 ul of volume. Preferably, the amplification and detection chambers may hold about 200 ul of volume. In some embodiments, an exonuclease is present in the amplification chamber or may be added to the amplification chamber. The exonuclease can clean up single stranded nucleic acids that are not the target. In some embodiments, primers for the target nucleic acid can be phosophorothioated in order to prevent degradation of the target nucleic acid in the presence of the exonuclease. In some embodiments, any of the devices disclosed herein can have a pH balancing well for balancing the pH of a sample. In some embodiments, in each of the above devices, the reporter is present in at least four-fold excess of total nucleic acids (target nucleic acids +non-target nucleic acids). Preferably the reporter is present in at least 10-fold excess of total nucleic acids. In some embodiments, the reporter is present in at least 4-fold, at least 5-fold at least 6-fold, at least 7-fold, at least 8-fold, at least 9-fold, at least 10-fold, at least 15-fold, at least 20-fold, at least 50-fold, at least 100-fold, from 1.5 to 100-fold, from 4 to 80-fold, from 4 to 10-fold, from 5 to 20-fold or from 4 to 15-fold excess of total nucleic acids. In some embodiments, any of the devices disclosed herein can carry out a DETECTR reaction with a limit of detection of at least 0.1 aM, at least 0.1 nM, at least 1 nM or from 0.1 aM to 1 nM. In some embodiments, the devices disclosed herein can carry out a DETECTR reaction with a positive predictive value of at least 75%, at least 80%, at least 85%, at least 90%, at least 92%, at least 95%, at least 97%, at least 99%, or 100%. In some embodiments, the devices disclosed herein can carry out a DETECTR reaction with a negative predictive value of at least 75%, at least 80%, at least 85%, at least 90%, at least 92%, at least 95%, at least 97%, at least 99%, or 100%. In some embodiments, spatial multiplexing in the above devices is carried out by having at least one, more than one, or every detection chamber in the device comprise a unique guide nucleic acid.

Workflows. A DETECTR reaction may be performed in a microfluidic device using many different workflows. In some embodiments, a workflow for measuring a buccal swab sample may comprise swabbing a cheek, adding the swab to a lysis solution, incubating the swab to lyse the sample, combining the lysed sample with reagents for amplification of a target nucleic acid, combining the amplified sample with DETCTR reagents, and incubating the sample to detect the target nucleic acid. In some embodiments, one or more of lysis, amplification, and detection may be performed in a microfluidic device (e.g., a microfluidic cartridge illustrated in FIG. 126A-B, FIG. 127A-B, FIG. 128A-D, FIG. 129A-D, FIG. 130A, FIG. 133, FIG. 150, FIG. 151, or FIG. 157-FIG. 167. In some embodiments, the workflow may comprise measuring a detectable signal indicative of the presence or absence of a target nucleic acid using a detection manifold (e.g., a detection manifold illustrated in FIG. 136A-B, FIG. 137B, FIG. 137C, FIG. 138A-B, FIG. 156, FIG. 168, or FIG. 172).

An example of a workflow for detecting a target nucleic acid is provided in FIG. 134. The cartridge may be loaded with a sample and reaction solutions. The amplification chamber may be heated to 60° C. and the sample may incubated in the amplification chamber for 30 minutes. The amplified sample may be pumped to the DETECTR reaction chambers, and the DETECTR reagents may be pumped to the DETECTR reaction chambers. The DETECTR reaction chambers may be heated to 37° C. and the sample may be incubated for 30 minutes. The fluorescence in the DETECTR reaction chambers may be measured in real time to produce a quantitative result.

An example of a workflow for detecting a target nucleic acid (e.g., a viral target nucleic acid) may comprise swabbing a cheek of a subject. The swab may be added to about 200 μL of a low-pH solution. In some embodiments, the swab may displace the solution so that the total volume is about 220 μL. The swab may be incubated in the low-pH solution for about a minute. In some embodiments, cells or viral capsids present on the swab may be lysed in the low-pH solution. A portion of the sample (5 μL) may be combined with about 45 μL of an amplification solution in an amplification chamber. The total volume within the chamber may be about 50 μL. The sample may be incubated in the amplification chamber for up to about 30 minutes at a temperature of from about 50° C. to about 65° C. to amplify the target nucleic acid the sample. In some embodiments, two aliquots of about 5 μL each of the amplified sample may be directed to two detection chambers where they are combined with about 95 μL each of a DETECTR reaction mix. The amplified sample may be incubated with the DETECTR reaction mix for up to about 10 minutes at about 37° C. in each of two detection chambers to detect the presence or absence of the target nucleic acid.

In some embodiments, a workflow for a DETECTR reaction performed in a microfluidic device may be implemented by a user. A user may collect a sample from a subject (e.g., a buccal swab or a nasal swab), place the sample in a lysis buffer, add the lysed sample to a microfluidic cartridge of the present disclosure, and insert the cartridge in a detection manifold of the present disclosure. In some embodiments, a user may add an unlysed sample to the microfluidic cartridge. In some embodiments, a workflow for a DETECTR reaction may be implemented in a microfluidic cartridge of the present disclosure. A microfluidic cartridge may comprise one or more reagents in one or more chambers to facilitate one or more of lysis, amplification, or detection of a target nucleic acid in a sample. In some embodiments, a workflow for a DETECTR reaction performed in a microfluidic device may be facilitated by a detection manifold. A detection manifold may provide one or more of heating control for an amplification reaction, a detection reaction, or both, solution movement control (e.g., pump control or valve control), illumination, or detection.

In some embodiments, a workflow for a DETECTR performed a microfluidic cartridge and facilitated by a user and a detection manifold may comprise steps of: 1) user loads sample into cartridge comprising one or more reagents, 2) user inserts cartridge into a detection manifold and presses a start button, 3) manifold energizes a solenoid to close a valve between a amplification chamber and a detection chamber, 4) manifold indicator LED turns on, 5) manifold turns on first heater to heat a first heating zone to 60° C. and second heater to heat a second heating zone to 37° C., 5) incubate sample in amplification chamber for 30 minutes in first heating zone to amplify sample, 6) manifold turns off first heater, 7) manifold de-energizes solenoid to open valve, 8) manifold turns on a first pump for 15 seconds to pump the amplified sample to the detection chamber, 9) manifold turns off first pump, 10) manifold turns on a second pump for 15 seconds to pump detection reagents from a detection reagent storage chamber to the detection chamber, 11) manifold turns off second pump, 12) incubate amplified sample and detection reagents in detection chamber for 30 minutes in second heating zone to perform detection reaction, 13) manifold indicator LED turns off, 14) manifold turns on illumination source and measures detectable signal produced by detection reaction.

An example of a workflow that may be performed in a microfluidic device, for example the microfluidic device shown in FIG. 159, and facilitated by a detection manifold, for example the detection manifold shown in FIG. 168, may comprise the following steps: 1) Add a swab containing a sample to chamber C2 while valves V1-V18 are closed, heater 1 is off, and heater 2 is off; 2) snap off the end of the swab and close the lid of the device; 3) suspend swab in lysis solution by opening valve V1 to facilitate flow of lysis solution from chamber C1 to chamber C2; 4) meter about 20 μL of lysate from chamber C2 to each of chambers C7-C10 by opening valve V2 and mix with contents from chambers C3-C6 by opening valves V3-V6; 5) close all valves and turn on heater 1 to incubate the samples in chambers C7-C10 at 60° C. to amplify; 6) turn off heater 1, meter about 10 μL of amplicon into each of chambers C19-C26 from chambers C7-C10 (2×10 μL from each chamber), and combine with the contents from each of chambers C11-C18 by opening valves V7-V18; 7) close all valves and turn on heater 2 to incubate the sample in chambers C19-C26 at 37° C. to perform CRISPR detection reaction; 8) detect the samples in chambers C19-C26 by illuminating at 470 nm and detecting at 520 nm during the incubation of step 7.

In some embodiments, a workflow performed in microfluidic device may comprise partitioning a sample into two or more chambers. A device may be configured to partition a sample into a plurality of portions. A device may be configured to transfer two portions of a partitioned sample into separate fluidic channels or chambers. A device may be configured to transfer a plurality of portions of a sample into a plurality of different fluidic channels or chambers. A device may be configured to perform reactions on individual portions of a partitioned sample. A device may be configured to partition a sample into 2 portions. A device may be configured to partition a sample into 3 portions. A device may be configured to partition a sample into 4 portions. A device may be configured to partition a sample into 5 portions. A device may be configured to partition a sample into 6 portions. A device may be configured to partition a sample into 7 portions. A device may be configured to partition a sample into 8 portions. A device may be configured to partition a sample into 9 portions. A device may be configured to partition a sample into 10 portions. A device may be configured to partition a sample into 12 portions. A device may be configured to partition a sample into 15 portions. A device may be configured to divide a sample into at least 20 portions. A device may be configured to partition a sample into at least 50 portions. A device may be configured to partition a sample into 100 portions. A device may be configured to partition a sample into 500 portions.

A device may be configured to perform a first reaction on a first portion of a sample and a second reaction on a second portion of a partitioned sample. A device may be configured to perform a different reaction on each portion of a partitioned sample. A device may be configured to perform sequential reactions on a sample or a portion of a sample. A device may be configured to perform a first reaction in a first chamber and a second reaction in a second chamber on a sample or portion of a sample.

A device may be configured to mix a sample with reagents. In some cases, a device mixes a sample with reagents by flowing the sample and reagents back and forth between a plurality of compartments. In some cases, a device mixes a sample with reagents by cascading the sample and reagents into a single compartment (e.g., by flowing both the sample and reagents into the compartment from above). In some cases, the mixing method performed by the device minimizes the formation of bubbles. In some cases, the mixing method performed by the device minimizes the sample loss or damage (e.g., protein precipitation).

A device may be configured to perform a plurality of reactions on a plurality of portions of a sample. In some cases, a device comprises a plurality of chambers each comprising reagents. In some cases, two chambers from among the plurality of reagent comprising chambers comprise different reagents. In some cases, a first portion and a second portion of a sample may be subjected to different reactions. In some cases, a first portion and a second portion of a sample may be subjected to the same reactions in the presence of different reporter molecules. In some cases, a first portion and a second portion of a sample may be subjected to the same detection method. In some cases, a first portion and a second portion of a sample may be subjected to different detection methods. In some cases, a plurality of portions of a sample may be detected separately (e.g., by a diode array that excites and detects fluorescence from each portion of a sample individually). In some cases, a plurality of portions of a sample may be detected simultaneously. For example, a device may partition a single sample into 4 portions, perform different amplification reactions on each portion, partition the products of each amplification reaction into two portions, perform different DETECTR reactions on each portion, and individually measure the progress of each DETECTR reaction.

A device may be configured to partition a small quantity of sample for a large number of different reactions or sequences of reactions. In some cases, a device may partition less than 1 ml of sample for a plurality of different reactions or sequences of reactions. In some cases, a device may partition less than 800 μl of sample for a plurality of different reactions or sequences of reactions. In some cases, a device may partition less than 600 μl of sample for a plurality of different reactions or sequences of reactions. In some cases, a device may partition less than 400 μl of sample for a plurality of different reactions or sequences of reactions. In some cases, a device may partition less than 200 μl of sample for a plurality of different reactions or sequences of reactions. In some cases, a device may partition less than 100 μl of sample for a plurality of different reactions or sequences of reactions. In some cases, a device may partition less than 50 μl of sample for a plurality of different reactions or sequences of reactions. In some cases, a device may partition less than 1 mg of sample for a plurality of different reactions or sequences of reactions. In some cases, a device may partition less than 800 μg of sample for a plurality of different reactions or sequences of reactions. In some cases, a device may partition less than 600 μg of sample for a plurality of different reactions or sequences of reactions. In some cases, a device may partition less than 400 μg of sample for a plurality of different reactions or sequences of reactions. In some cases, a device may partition less than 200 μg of sample for a plurality of different reactions or sequences of reactions. In some cases, a device may partition less than 100 μg of sample for a plurality of different reactions or sequences of reactions. In some cases, a device may partition less than 50 μg of sample for a plurality of different reactions or sequences of reactions. In some cases, a device may partition less than 20 μg of sample for a plurality of different reactions or sequences of reactions. In some cases, a device may partition less than 10 μg of sample for a plurality of different reactions or sequences of reactions. In some cases, a device may partition less than 1 μg of sample for a plurality of different reactions or sequences of reactions. In some cases, a device may partition less than 800 ng of sample for a plurality of different reactions or sequences of reactions. In some cases, a device may partition less than 600 ng of sample for a plurality of different reactions or sequences of reactions. In some cases, a device may partition less than 400 ng of sample for a plurality of different reactions or sequences of reactions. In some cases, a device may partition less than 200 ng of sample for a plurality of different reactions or sequences of reactions. In some cases, a device may partition less than 100 ng of sample for a plurality of different reactions or sequences of reactions. In some cases, a device may partition less than 50 ng of sample for a plurality of different reactions or sequences of reactions. In some cases, the sample may comprise nucleic acid. In some cases, the sample may comprise cells. In some cases, the sample may comprise proteins. In some cases, the plurality of different reactions or sequences of reactions may comprise 2 or more different reactions or sequences of reactions. In some cases, the plurality of different reactions or sequences of reactions may comprise 3 or more different reactions or sequences of reactions. In some cases, the plurality of different reactions or sequences of reactions may comprise 4 or more different reactions or sequences of reactions. In some cases, the plurality of different reactions or sequences of reactions may comprise 5 or more different reactions or sequences of reactions. In some cases, the plurality of different reactions or sequences of reactions may comprise 10 or more different reactions or sequences of reactions. In some cases, the plurality of different reactions or sequences of reactions may comprise 20 or more different reactions or sequences of reactions. In some cases, the plurality of different reactions or sequences of reactions may comprise 50 or more different reactions or sequences of reactions. In some cases, the plurality of different reactions or sequences of reactions may comprise 100 or more different reactions or sequences of reactions. In some cases, the plurality of different reactions or sequences of reactions may comprise 500 or more different reactions or sequences of reactions. In some cases, the plurality of different reactions or sequences of reactions may comprise 1000 or more different reactions or sequences of reactions. In some cases, a first reaction or sequence of reactions and a second reaction or sequence of reactions detect two different nucleic acid sequences. In some cases, each reaction or sequence of reactions from among a plurality of different reactions or sequences of reactions detects a different nucleic acid sequence. For example, a device may be configured to perform 40 different sequences of reactions designed to detect 40 different nucleic acid sequences from a single sample comprising 200 ng DNA (e.g., 200 ng DNA from a buccal swab). In such a case, each of the 40 different nucleic acid sequences could be used to determine the presence of a particular virus in the sample.

In some cases, a device is configured to automate a step. In some cases, a device automates a sample partitioning step. In some cases, a device automates a reaction step (e.g., by mixing a sample with reagents and heating to a temperature for a defined length of time). In some cases, the device automates every step following sample input. In some cases, a device may automate a plurality of reactions on a single input sample. In some cases, a device may automate, detect, and provide results for a plurality of reactions on a single input sample. In some cases, a device may automate, detect, and provide results for a plurality of reactions on a single sample in less than 2 hours. For example, a device may automate 100 separate amplification and DETECTR reactions on a sample comprising 400 ng DNA, detect and then provide the results of the reactions in less than 2 hours. In some cases, a device may automate, detect, and provide results for a plurality of reactions on a single sample in less than 1 hour. In some cases, a device may automate, detect, and provide results for a plurality of reactions on a single sample in less than 40 minutes. In some cases, a device may automate, detect, and provide results for a plurality of reactions on a single sample in less than 20 minutes. In some cases, a device may automate, detect, and provide results for a plurality of reactions on a single sample in less than 10 minutes. In some cases, a device may automate, detect, and provide results for a plurality of reactions on a single sample in less than 5 minutes. In some cases, a device may automate, detect, and provide results for a plurality of reactions on a single sample in less than 2 minutes.

Microfluidic devices and detection manifolds for detection of viral infections. A microfluidic device of the present disclosure (e.g., a microfluidic device illustrated in FIG. 126A-B, FIG. 127A-B, FIG. 128A-D, FIG. 129A-D, FIG. 130A, FIG. 133, FIG. 151, FIG. 154, or FIG. 157-FIG. 167) may be used to detect the presence or absence of an influenza virus (e.g., an influenza A virus or an influenza B virus) in a biological sample. Detection of the influenza virus may be facilitated by a detection manifold (e.g., a detection manifold illustrated in FIG. 136A-B, FIG. 137B, FIG. 137C, FIG. 138A-B, FIG. 156, FIG. 168, or FIG. 172). A biological sample may be collected from a subject, for example via a nasal swab or a buccal swab, and introduced into an amplification chamber of the microfluidic device. The chamber may comprise lysis buffer, amplification reagents, or both. In some embodiments, the biological sample may be contacted with a lysis buffer prior to introduction into the amplification chamber. In some embodiments, the amplification reagents may be introduced into the amplification chamber from an amplification reagent storage chamber. Introduction of the amplification reagents may be controlled by actuating a pump, a valve, or both via the detection manifold. The amplification reagents may comprise primers to amplify a target nucleic acid present in the influenza viral genome. If the target nucleic acid is present in the sample, the target nucleic acid may be amplified (e.g., by TMA, HDA, cHDA, SDA, LAMP, EXPAR, RCA, LCR, SMART, SPIA, MDA, NASBA, HIP, NEAR, or IMDA). The first chamber may be heated by the detection manifold. The amplified sample may be introduced into a detection chamber by actuating a pump, a valve, or both via the detection manifold. The amplified sample may pass through a sample metering channel. Detection reagents may be introduced into the detection channel from a detection reagent storage chamber by actuating a pump, a valve, or both via the detection manifold. The detection reagents may pass through a sample metering channel, a resistance channel, or both. The detection reagents may comprise a programmable nuclease, a guide nucleic acid directed to the target nucleic acid, and a labeled detector nucleic acid. A detection reaction may be performed in the detection channel by heating the detection channel via the detection manifold. The presence or absence of the target nucleic acid associated with the influenza virus may be detected in the detection channel using the detection manifold. The presence or absence of the influenza virus may be determined by measuring a detectable signal produced by cleavage of the detector nucleic acid by the programmable nuclease upon binding to the target nucleic acid.

A microfluidic device of the present disclosure (e.g., a microfluidic device illustrated in FIG. 126A-B, FIG. 127A-B, FIG. 128A-D, FIG. 129A-D, FIG. 130A, FIG. 133, FIG. 151, FIG. 154, or FIG. 157-FIG. 167) may be used to detect the presence or absence of a coronavirus (e.g., a SARS-CoV-2 virus, a SARS-CoV virus, a MERS-CoV virus, a combination thereof, or a combination of any coronavirus strain and one or more other viruses or bacteria) in a biological sample. Detection of the coronavirus may be facilitated by a detection manifold (e.g., a detection manifold illustrated in FIG. 136A-B, FIG. 137B, FIG. 137C, FIG. 138A-B, FIG. 156, FIG. 168, or FIG. 172). A biological sample may be collected from a subject, for example via a nasal swab or a buccal swab, and introduced into an amplification chamber of the microfluidic device. The chamber may comprise lysis buffer, amplification reagents, or both. In some embodiments, the biological sample may be contacted with a lysis buffer prior to introduction into the amplification chamber. In some embodiments, the amplification reagents may be introduced into the amplification chamber from an amplification reagent storage chamber. Introduction of the amplification reagents may be controlled by actuating a pump, a valve, or both via the detection manifold. The amplification reagents may comprise primers to amplify a target nucleic acid present in the coronavirus genome. If the target nucleic acid is present in the sample, the target nucleic acid may be amplified (e.g., by TMA, HDA, cHDA, SDA, LAMP, EXPAR, RCA, LCR, SMART, SPIA, MDA, NASBA, HIP, NEAR, or IMDA). The first chamber may be heated by the detection manifold. The amplified sample may be introduced into a detection chamber by actuating a pump, a valve, or both via the detection manifold. The amplified sample may pass through a sample metering channel. Detection reagents may be introduced into the detection channel from a detection reagent storage chamber by actuating a pump, a valve, or both via the detection manifold. The detection reagents may pass through a sample metering channel, a resistance channel, or both. The detection reagents may comprise a programmable nuclease, a guide nucleic acid directed to the target nucleic acid, and a labeled detector nucleic acid. A detection reaction may be performed in the detection channel by heating the detection channel via the detection manifold. The presence or absence of the target nucleic acid associated with the coronavirus may be detected in the detection channel using the detection manifold. The presence or absence of the coronavirus may be determined by measuring a detectable signal produced by cleavage of the detector nucleic acid by the programmable nuclease upon binding to the target nucleic acid.

A microfluidic device of the present disclosure (e.g., a microfluidic device illustrated in FIG. 126A-B, FIG. 127A-B, FIG. 128A-D, FIG. 129A-D, FIG. 130A, FIG. 133, FIG. 151, FIG. 154, or FIG. 157-FIG. 167) may be used to detect the presence or absence of a respiratory syncytial virus in a biological sample. Detection of the respiratory syncytial virus may be facilitated by a detection manifold (e.g., a detection manifold illustrated in FIG. 136A-B, FIG. 137B, FIG. 137C, FIG. 138A-B, FIG. 156, FIG. 168, or FIG. 172). A biological sample may be collected from a subject, for example via a nasal swab or a buccal swab, and introduced into an amplification chamber of the microfluidic device. The chamber may comprise lysis buffer, amplification reagents, or both. In some embodiments, the biological sample may be contacted with a lysis buffer prior to introduction into the amplification chamber. In some embodiments, the amplification reagents may be introduced into the amplification chamber from an amplification reagent storage chamber. Introduction of the amplification reagents may be controlled by actuating a pump, a valve, or both via the detection manifold. The amplification reagents may comprise primers to amplify a target nucleic acid present in the respiratory syncytial viral genome. If the target nucleic acid is present in the sample, the target nucleic acid may be amplified (e.g., by TMA, HDA, cHDA, SDA, LAMP, EXPAR, RCA, LCR, SMART, SPIA, MDA, NASBA, HIP, NEAR, or IMDA). The first chamber may be heated by the detection manifold. The amplified sample may be introduced into a detection chamber by actuating a pump, a valve, or both via the detection manifold. The amplified sample may pass through a sample metering channel. Detection reagents may be introduced into the detection channel from a detection reagent storage chamber by actuating a pump, a valve, or both via the detection manifold. The detection reagents may pass through a sample metering channel, a resistance channel, or both. The detection reagents may comprise a programmable nuclease, a guide nucleic acid directed to the target nucleic acid, and a labeled detector nucleic acid. A detection reaction may be performed in the detection channel by heating the detection channel via the detection manifold. The presence or absence of the target nucleic acid associated with the respiratory syncytial virus may be detected in the detection channel using the detection manifold. The presence or absence of the respiratory syncytial virus may be determined by measuring a detectable signal produced by cleavage of the detector nucleic acid by the programmable nuclease upon binding to the target nucleic acid.

### Kit

Disclosed herein are kits fluidic devices, and systems for use to detect a target nucleic acid. In some embodiments, the kit comprises the reagents and the support medium. The reagent may be provided in a reagent chamber or on the support medium. Alternatively, the reagent may be placed into the reagent chamber or the support medium by the individual using the kit. Optionally, the kit further comprises a buffer and a dropper. The reagent chamber be a test well or container. The opening of the reagent chamber may be large enough to accommodate the support medium. The buffer may be provided in a dropper bottle for ease of dispensing. The dropper can be disposable and transfer a fixed volume. The dropper can be used to place a sample into the reagent chamber or on the support medium.

In some embodiments, a kit for detecting a target nucleic acid comprising a support medium; a guide nucleic acid targeting a target nucleic acid segment; a programmable nuclease capable of being activated when complexed with the guide nucleic acid and the target nucleic acid segment; and a single stranded detector nucleic acid comprising a detection moiety, wherein the detector nucleic acid is capable of being cleaved by the activated nuclease, thereby generating a first detectable signal.

In some embodiments, a kit for detecting a target nucleic acid comprising a PCR plate; a guide nucleic acid targeting a target nucleic acid segment; a programmable nuclease capable of being activated when complexed with the guide nucleic acid and the target nucleic acid segment;

and a single stranded detector nucleic acid comprising a detection moiety, wherein the detector nucleic acid is capable of being cleaved by the activated nuclease, thereby generating a first detectable signal. The wells of the PCR plate can be pre-aliquoted with the guide nucleic acid targeting a target nucleic acid segment, a programmable nuclease capable of being activated when complexed with the guide nucleic acid and the target sequence, and at least one population of a single stranded detector nucleic acid comprising a detection moiety. A user can thus add the biological sample of interest to a well of the pre-aliquoted PCR plate and measure for the detectable signal with a fluorescent light reader or a visible light reader.

In some instances, such kits may include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In one embodiment, the containers are formed from a variety of materials such as glass, plastic, or polymers.

The kit or systems described herein contain packaging materials. Examples of packaging materials include, but are not limited to, pouches, blister packs, bottles, tubes, bags, containers, bottles, and any packaging material suitable for intended mode of use.

A kit typically includes labels listing contents and/or instructions for use, and package inserts with instructions for use. A set of instructions will also typically be included. In one embodiment, a label is on or associated with the container. In some instances, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

### Stability

Disclosed herein are stable compositions of the reagents and the programmable nuclease system for use in the methods as discussed above. The reagents and programmable nuclease system described herein may be stable in various storage conditions including refrigerated, ambient, and accelerated conditions. Disclosed herein are stable reagents. The stability may be measured for the reagents and programmable nuclease system themselves or the reagents and programmable nuclease system present on the support medium.

In some instances, stable as used herein refers to a reagents having about 5% w/w or less total impurities at the end of a given storage period. Stability may be assessed by HPLC or any other known testing method. The stable reagents may have about 10% w/w, about 5% w/w, about 4% w/w, about 3% w/w, about 2% w/w, about 1% w/w, or about 0.5% w/w total impurities at the end of a given storage period.

In some embodiments, stable as used herein refers to a reagents and programmable nuclease system having about 10% or less loss of detection activity at the end of a given storage period and at a given storage condition. Detection activity can be assessed by known positive sample using a known method. Alternatively or combination, detection activity can be assessed by the sensitivity, accuracy, or specificity. In some embodiments, the stable reagents has about 10%, about 9%, about 8%, about 7%, about 6%, about 5%, about 4%, about 3%, about 2%, about 1%, or about 0.5% loss of detection activity at the end of a given storage period.

In some embodiments, the stable composition has zero loss of detection activity at the end of a given storage period and at a given storage condition. The given storage condition may comprise humidity of equal to or less than 10%, 20%, 30%, 40%, 50%, 60%, 70%, 80%, 90%, or 100% relative humidity. The controlled storage environment may comprise humidity between 0% and 50% relative humidity, 0% and 40% relative humidity, 0% and 30% relative humidity, 0% and 20% relative humidity, or 0% and 10% relative humidity. The controlled storage environment may comprise temperatures of −100° C., −80° C., −20° C., 4° C., about 25° C. (room temperature), or 40° C. The controlled storage environment may comprise temperatures between −80° C. and 25° C., or −100° C. and 40° C. The controlled storage environment may protect the system or kit from light or from mechanical damage. The controlled storage environment may be sterile or aseptic or maintain the sterility of the light conduit. The controlled storage environment may be aseptic or sterile.

In some cases, reagents may be stored in a capillary. A capillary may be a glass capillary. In some cases, a capillary provides a controlled storage environment. A capillary may also be stored within a controlled storage environment. A capillary can store a solution containing a reagent. A capillary can store a reagent in a dry form. A capillary can be loaded with a solution containing a reagent and then be dried to yield a capillary containing a dried or powdered form of the reagent. A dried or powdered reagent may be hydrated or dissolved by filling the capillary with a solution (e.g., buffer). A reagent within a capillary may be stable when stored at room temperature. A reagent within a capillary may stable when stored at (e.g., 37° C.). A reagent within a capillary may be stable when stored below room temperature (e.g., 4 37° C.). A reagent within a capillary may be stable when stored for 1, 2, 3, 4, 5, 6, 7, 8, 9, 10, 11 or 12 months. A reagent stored within a capillary may be stable when stored for longer than a year. A reagent stored within a capillary may retain greater than 25%, 30%, 35%, 40%, 45%, 50%, 55%, 60%, 65%, 70%, 75%, 80%, 85%, 90%, 95% or 100% of its activity.

A capillary can contain an enzyme in dried form or in solution. A capillary can contain a programmable nuclease in dried form or in solution. A capillary can contain a nucleic acid in dried form or in solution. A capillary can contain an ribonucleoprotein in dried form or in solution. A capillary can contain a dye in dried form or in solution. A capillary can contain a buffer (e.g., a lysis buffer) in dried form or in solution. A capillary can contain amplification reagents in dried form or in solution.

A reagent may be removed from a capillary by flowing a solution through the capillary. A reagent may be removed from a capillary by applying pressure (e.g., hydraulic or pneumatic pressure) to an open end of the capillary. A reagent may be removed from a capillary by breaking the capillary. A capillary may be positioned so that its contents elute due to gravity. A capillary may be open at both ends. A capillary may be sealed at one or two ends.

A capillary may have an internal volume of less than 1 μl. A capillary can have an internal volume of 1 μl. A capillary can have an internal volume of 2 μl. A capillary can have an internal volume of 3 μl. A capillary can have an internal volume of 4 μl. A capillary can have an internal volume of 5 μl. A capillary can have an internal volume of between 5 and 10 μl. A capillary can have an internal volume of between 10 and 20 μl. A capillary can have an internal volume of between 20 and 30 μl. A capillary can have an internal volume of between 30 and 40 μl. A capillary can have an internal volume of between 40 and 50 μl. A capillary can have an internal volume of between 50 and 60 μl. A capillary can have an internal volume of between 60 and 70 μl. A capillary can have an internal volume of between 70 and 80 μl. A capillary can have an internal volume of between 80 and 90 μl. A capillary can have an internal volume of between 90 and 100 μl. A capillary can have an internal volume of greater than 100 μl.

The kit or system can be packaged to be stored for extended periods of time prior to use. The kit or system may be packaged to avoid degradation of the kit or system. The packaging may include desiccants or other agents to control the humidity within the packaging. The packaging may protect the kit or system from mechanical damage or thermal damage. The packaging may protect the kit or system from contamination of the reagents and programmable nuclease system. The kit or system may be transported under conditions similar to the storage conditions that result in high stability of the reagent or little loss of reagent activity. The packaging may be configured to provide and maintain sterility of the kit or system. The kit or system can be compatible with standard manufacturing and shipping operations.

### Target Amplification and Detection

A number of target amplification and detection methods are consistent with the methods, compositions, reagents, enzymes, and kits disclosed herein. As described herein, a target nucleic acid may be detected using a DNA-activated programmable RNA nuclease (e.g., a Cas13), a DNA-activated programmable DNA nuclease (e.g., a Cas12), or an RNA-activated programmable RNA nuclease (e.g., a Cas13) and other reagents disclosed herein (e.g., RNA components). The target nucleic acid may be detected using DETECTR, as described herein. The target nucleic acid may be an RNA, reverse transcribed RNA, DNA, DNA amplicon, amplified DNA, synthetic nucleic acids, or nucleic acids found in biological or environmental samples. In some cases, the target nucleic acid is amplified prior to or concurrent with detection. In some cases, the target nucleic acid is reverse transcribed prior to amplification. The target nucleic acid may be amplified via loop mediated isothermal amplification (LAMP) of a target nucleic acid sequence. In some cases, the nucleic acid is amplified using LAMP coupled with reverse transcription (RT-LAMP). The LAMP amplification may be performed independently, or the LAMP amplification may be coupled to DETECTR for detection of the target nucleic acid. The RT-LAMP amplification may be performed independently, or the RT-LAMP amplification may be coupled to DETECTR for detection of the target nucleic acid. The DETECTR reaction may be performed using any method consistent with the methods disclosed herein.

### Amplification and Detection Reaction Mixtures

In some embodiments, a LAMP amplification reaction comprises a plurality of primers, dNTPs, and a DNA polymerase. LAMP may be used to amplify DNA with high specificity under isothermal conditions. The DNA may be single stranded DNA or double stranded DNA. In some cases, a target nucleic acid comprising RNA may be reverse transcribed into DNA using a reverse transcriptase prior to LAMP amplification. A reverse transcription reaction may comprise primers, dNTPs, and a reverse transcriptase. In some cases, the reverse transcription reaction and the LAMP amplification reaction may be performed in the same reaction. A combined RT-LAMP reaction may comprise LAMP primers, reverse transcription primers, dNTPs, a reverse transcriptase, and a DNA polymerase. In some case, the LAMP primers may comprise the reverse transcription primers.

A DETECTR reaction to detect the target nucleic acid sequence may comprise a guide nucleic acid comprising a segment that is reverse complementary to a segment of the target nucleic acid and a programmable nuclease. The programmable nuclease when activated, as described elsewhere herein, exhibits sequence-independent cleavage of a reporter (e.g., a nucleic acid comprising a moiety that becomes detectable upon cleavage of the nucleic acid by the programmable nuclease). The programmable nuclease is activated upon the guide nucleic acid hybridizing to the the target nucleic acid. A combined LAMP DETECTR reaction may comprise a plurality of primers, dNTPs, a DNA polymerase, a guide nucleic acid, a programmable nuclease, and a substrate nucleic acid. A combined RT-LAMP DETECTR reaction may comprise LAMP primers, reverse transcription primers, dNTPs, a reverse transcriptase, a DNA polymerase, a guide nucleic acid, a programmable nuclease, and a substrate nucleic acid. In some case, the LAMP primers may comprise the reverse transcription primers. LAMP and DETECTR can be carried out in the same sample volume. LAMP and DETECTR can be carried out concurrently in separate sample volumes or in the same sample volume. RT-LAMP and DETECTR can be carried out in the same sample volume. RT-LAMP and DETECTR can be carried out concurrently in separate sample volumes or in the same sample volume.

### Primer Design for LAMP Amplification

A LAMP reaction may comprise a plurality of primers. A plurality of primers are designed to amplify a target nucleic acid sequence, which is shown in FIG. 61 relative to various regions of a double stranded nucleic acid. The primers can anneal to or have sequences corresponding to these various regions. As shown in FIG. 61, the target nucleic acid is 5′ of an F1c region, the F1c region is 5′ of the F2c region, and the F2c region is 5′ of the F3c region. Additionally, the B1 region is 3′ of the B2 region, and the B2 region is 3′ of the B3 region. The F3c, F2c, F1c, B1, B2, and B3 regions are shown on the lower strand in FIG. 61. An F3 region is a sequence reverse complementary to the F3c region. An F2 region is a sequence reverse complementary to the F2c region. An Fl region is a sequence reverse complementary to the F1c region. The B1c region is a sequence reverse complementary to a B1 region. The B2c region is a sequence reverse complementary to a B2 region. The B3c region is a sequence reverse complementary to a B3 region. The target nucleic acid may be 5′ of the F1c region and 3′ of the B1 region, as shown in the top configuration of FIG. 61. The target nucleic acid may be 5′ of the B1c region and 3′ of the F1 region, as shown in the bottom configuration of FIG. 61. In some embodiments, the target nucleic acid may be 5′ of the F2c region and 3′ of the F1c region. In some embodiments, the target nucleic acid may be 5′ of the B2c region and 3′ of the B1c region. In some embodiments, the target nucleic acid sequence may be 5′ of the B1 region and 3′ of the B2 region. In some embodiments, the target nucleic acid sequence may be 5′ of the F1 region and 3′ of the F2 region.

FIG. 61 also shows the structure and directionality of the various primers. The forward outer primer has a sequence of the F3 region. Thus, the forward outer primer anneals to the F3c region. The backward outer primer has a sequence of the B3 region. Thus, the backward outer primer anneals to the B3c region. The forward inner primer has a sequence of the F1c region 5′ of a sequence of the F2 region. Thus, the F2 region of the forward inner primer anneals to the F2c region and the amplified sequence forms a loop held together via hybridization of the sequence of the F1c region in the forward inner primer and the Fl region. The backward inner primer has a sequence of a B1c region 5′ of a sequence of the B2 region. Thus, the B2 region of the backward inner primer anneals to the B2c region and the amplified sequence forms a loop held together via hybridization of the sequence of the B1c region of the backward inner primer and the B1 region of the target strand.

Further, as shown in FIG. 61, the plurality of primers may additionally include a loop forward primer (LF) and/or a loop backward primer (LB). LF is positioned 3′ of the F1c region and 5′ of the F2c region. LB is positioned 5′ of the B2c region and 3′ of the B1c region. The F1, F1c, F2, F2c, F3, F3c, B1, B1c, B2, B2c, B3, and/or B3c regions are illustrated in various arrangements relative to the target nucleic acid, the PAM, and the guide RNA (gRNA), as shown in any one of FIG. 61-FIG. 63 or FIG. 71-FIG. 72. The target nucleic acid may be within the nucleic acid strand comprising the B1, B2, B3, LF, F1c, F2c, F3c, and LBc regions. The target nucleic acid may be within the nucleic acid strand comprising the F1, F2, F3, LB, B1c, B2c, B3c, and LFc regions.

A set of LAMP primers may be designed for use in combination with a DETECTR reaction. The nucleic acid may comprise a region (e.g., a target nucleic acid), to which a guide RNA hybridizes. All or part of the guide RNA sequence may be reverse complementary to all or part of the target sequence. The target nucleic acid sequence may be adjacent to a protospacer adjacent motif (PAM) 3′ of the target nucleic acid sequence. The PAM may promote interaction the programmable nuclease with the target nucleic acid. The target nucleic acid sequence may be adjacent to a protospacer flanking site (PFS) 3′ of the target nucleic acid sequence. The PFS may promote interaction the programmable nuclease with the target nucleic acid. One or more of the guide RNA, the PAM or PFS, or the target nucleic acid sequence may be specifically positioned with respect to one or more of the F1, F1c, F2, F2c, F3, F3c, LF, LFc, LB, LBc, B1, B1c, B2, B2c, B3, and/or B3c regions.

In some cases, the guide RNA is reverse complementary to a sequence of the target nucleic acid, which is between an F1c region and a B1 region, as in FIG. 62A. In some cases, the guide RNA is reverse complementary to a sequence of the target nucleic acid, which is between a B1c region and an F1 region.

In some cases, the guide RNA is partially reverse complementary to a sequence of the target nucleic acid, which is between an F1c region and a B1 region, as in FIG. 62B. In some cases, the guide RNA is partially reverse complementary to a sequence of the target nucleic acid, which is between a B1c region and an F1 region. For example, the target nucleic acid comprises a sequence between an F1c region and a B1 region or a B1c region and an F1 region that is reverse complementary to at least 60% of a guide nucleic acid. In another example, the target nucleic acid comprises a sequence between an F1c region and a B1 region that is reverse complementary to at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, from 5% to 100%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 95%, or from 95% to 100% of a guide nucleic acid. In this arrangement, the guide RNA is not reverse complementary to the forward inner primer or the backward inner primer shown in FIG. 61.

In some cases, the guide RNA is reverse complementary to no more than 50%, no more than 40%, no more than 35%, no more than 30%, no more than 25%, no more than 20%, no more than 15%, no more than 10%, or no more than 5% of the forward inner primer, the backward inner primer, or a combination thereof the sequence between the F1c region and the B1 region or the sequence between the B1c region and the F1 region is at least 50%, at least 70%, at least 75%, at least 80%, at least 85%, at least 90%, at least 92%, at least 95%, at least 99%, or 100% reverse complementary to the guide nucleic acid sequence. In some cases, the guide nucleic acid has a sequence reverse complementary to no more than 50%, no more than 40%, no more than 35%, no more than 30%, no more than 25%, no more than 20%, no more than 15%, no more than 10%, or no more than 5% of the forward inner primer, the backward inner primer, the forward outer primer, the backward outer primer, or any combination thereof. In some cases, the guide nucleic acid sequence has a sequence reverse complementary to no more than 50%, no more than 40%, no more than 35%, no more than 30%, no more than 25%, no more than 20%, no more than 15%, no more than 10%, or no more than 5% of a sequence of an F3c region, an F2c region, the F1c region, the B1c region, an B2c region, an B3c region, or any combination thereof.

In some cases, the region corresponding to the guide RNA sequence does not overlap or hybridize to any of the primers and may further not overlap with or hybridize to any of the regions shown in FIG. 61-FIG. 63 and FIG. 71-FIG. 72.

In some cases, all or a portion of the guide nucleic acid is reverse complementary to a sequence of the target nucleic acid in a loop region. For example, all or a portion of the sequence of the target nucleic acid that hybridizes to the gRNA may be located between the B1c and B2 regions, as shown in FIG. 62C. In another example, all or a portion of the sequence of the target nucleic acid that hybridizes to the gRNA may be located between the F2c and F1c regions, as shown in FIG. 62D. In some cases, all or a portion of the sequence of the target nucleic acid that hybridizes to the gRNA may be located between the F1 and F2 regions. In some cases, all or a portion of the sequence of the target nucleic acid that hybridizes to the gRNA may be located between the B2c and B1c regions.

In some cases, a LAMP primer set may be designed using a commercially available primer design software. A LAMP primer set may be designed for use in combination with a DETECR reaction, a reverse transcription reaction, or both. In some cases, a LAMP primer set may be designed using distributed ledger technology (DLT), artificial intelligence (AI), extended reality (XR) and quantum computing, commonly called “DARQ.” In some cases, a LAMP primer set may be designed using quenching of unincorporated amplification signal reporters (QUASR) (Ball et al., Anal Chem. 2016 Apr. 5; 88(7):3562-8. doi: 10.1021/acs.analchem.5b04054. Epub 2016 Mar. 24.). These methods of designing a set of LAMP primers are provided by way of example only; other methods of designing a set of LAMP primers may be readily apparent to one skilled in the art and may be employed in any of the compositions, kits and methods described herein. Exemplary sets of LAMP primers for use in a combined RT-LAMP DETECTR reaction or LAMP-DETECTR to detect the presence of a nucleic acid sequence corresponding to a respiratory syncytial virus (RSV), an influenza A virus (IAV), an influenza B virus (IAV), or a HERC2 SNP are provided in TABLE 6.

TABLE 6
Exemplary LAMP Primers

| SEQ ID NO: | Primer Name | Primer Set | Sequence |
|---|---|---|---|
| SEQ ID NO: 148 | F3 RSV-A- | #1 | TGGAACAAGTTGTGGAGG |
| | set13 | | |
| SEQ ID NO: 149 | B3 RSV-A- | #1 | TGCAGCATCATATAGATCTTGA |
| | set13 | | |
| SEQ ID NO: 150 | FIP RSV-A- | #1 | TAGTGATGCTTTTGGGTTGTTCAAT |
| | set13 | | TGTATGAGTATGCTCAAAAATTGG |
| SEQ ID NO: 151 | BIP RSV-A- | #1 | GTGTAGTATTGGGCAATGCTGCTC |
| | set13 | | CTTGGTGTACCTCTGT |
| SEQ ID NO: 152 | LF RSV-A- | #1 | TATGGTAGAATCCTGCTTCTCC |
| | set13 | | |
| SEQ ID NO: 153 | LB RSV-A- | #1 | TGGCCTAGGCATAATGGGAGA |
| | set13 | | |
| SEQ ID NO: 154 | F3 RSV-A- | #2 | AACAAGTTGTGGAGGTGTA |
| | set14 | | |
| SEQ ID NO: 155 | B3 RSV-A- | #2 | CCATTTTCTTTGAGTTGTTCAG |
| | set14 | | |
| SEQ ID NO: 156 | FIP RSV-A- | #2 | TAGTGATGCTTTTGGGTTGTTCAA |
| | set14 | | GAGTATGCTCAAAAATTGGGTG |
| SEQ ID NO: 157 | BIP RSV-A- | #2 | GTATTGGGCAATGCTGCTGGCATA |
| | set14 | | TAGATCTTGATTCCTTGGTG |
| SEQ ID NO: 158 | LF RSV-A- | #2 | ATATGGTAGAATCCTGCTTCTC |
| | set14 | | |
| SEQ ID NO: 159 | LB RSV-A- | #2 | CCTAGGCATAATGGGAGAATAC |
| | set14 | | |
| SEQ ID NO: 154 | F3 RSV-A- | #3 | AACAAGTTGTGGAGGTGTA |
| | set15 | | |
| SEQ ID NO: 155 | B3 RSV-A- | #3 | CCATTTTCTTTGAGTTGTTCAG |
| | set15 | | |
| SEQ ID NO: 160 | FIP RSV-A- | #3 | ATAGTGATGCTTTTGGGTTGTTCA |
| | set15 | | AGTATGCTCAAAAATTGGGTG |
| SEQ ID NO: 161 | BIP RSV-A- | #3 | GCTGCTGGCCTAGGCATAATGCAT |
| | set15 | | CATATAGATCTTGATTCCTT |
| SEQ ID NO: 380 | LF RSV-A- | #3 | TATATGGTAGAATCCTGCTTCTC |
| | set15 | | |
| SEQ ID NO: 162 | LB RSV-A- | #3 | GGGAGAATACAGAGGTACAC |
| | set15 | | |
| SEQ ID NO: 163 | F3 RSV-A- | #4 | GGGTCTTAGCAAAATCAGTT |
| | set16 | | |
| SEQ ID NO: 149 | B3 RSV-A- | #4 | TGCAGCATCATATAGATCTTGA |
| | set16 | | |
| SEQ ID NO: 164 | FIP RSV-A- | #4 | GAATCCTGCTTCTCCACCCAATTG |
| | set16 | | ACACGCTAGTGTACAAGC |
| SEQ ID NO: 151 | BIP RSV-A- | #4 | GTGTAGTATTGGGCAATGCTGCTC |
| | set16 | | CTTGGTGTACCTCTGT |
| SEQ ID NO: 165 | LF RSV-A- | #4 | CCTCCACAACTTGTTCCATTTCT |
| | set16 | | |
| SEQ ID NO: 166 | LB RSV-A- | #4 | TGGCCTAGGCATAATGGGAG |
| | set16 | | |
| SEQ ID NO: 167 | F3 RSV-A- | #5 | AAGCAGAAATGGAACAAGTT |
| | set17 | | |
| SEQ ID NO: 155 | B3 RSV-A- | #5 | CCATTTTCTTTGAGTTGTTCAG |
| | set17 | | |
| SEQ ID NO: 168 | FIP RSV-A- | #5 | TAGTGATGCTTTTGGGTTGTTCAGT |
| | set17 | | GGAGGTGTATGAGTATGC |
| SEQ ID NO: 169 | BIP RSV-A- | #5 | GTAGTATTGGGCAATGCTGCTGAT |
| | set17 | | ATAGATCTTGATTCCTTGGTG |
| SEQ ID NO: 170 | LF RSV-A- | #5 | TGCTTCTCCACCCAATTTTTGA |
| | set17 | | |
| SEQ ID NO: 171 | LB RSV-A- | #5 | GCCTAGGCATAATGGGAGAATAC |
| | set17 | | |
| SEQ ID NO: 163 | F3 RSV-A- | #6 | GGGTCTTAGCAAAATCAGTT |
| | set18 | | |
| SEQ ID NO: 149 | B3 RSV-A- | #6 | TGCAGCATCATATAGATCTTGA |
| | set18 | | |
| SEQ ID NO: 172 | FIP RSV-A- | #6 | GAATCCTGCTTCTCCACCCAGACA |
| | set18 | | CGCTAGTGTACAAGC |
| SEQ ID NO: 151 | BIP RSV-A- | #6 | GTGTAGTATTGGGCAATGCTGCTC |
| | set18 | | CTTGGTGTACCTCTGT |
| SEQ ID NO: 165 | LF RSV-A- | #6 | CCTCCACAACTTGTTCCATTTCT |
| | set18 | | |
| SEQ ID NO: 166 | LB RSV-A- | #6 | TGGCCTAGGCATAATGGGAG |
| | set18 | | |
| SEQ ID NO: 173 | F3 RSV-A- | #7 | TACACAGCTGCTGTTCAA |
| | set19 | | |
| SEQ ID NO: 174 | B3 RSV-A- | #7 | GGTAAATTTGCTGGGCATT |
| | set19 | | |
| SEQ ID NO: 175 | FIP RSV-A- | #7 | TTGGAACATGGGCACCCATAAATG |
| | set19 | | TCCTAGAAAAAGACGATG |
| SEQ ID NO: 176 | BIP RSV-A- | #7 | CTAGTGAAACAAATATCCACACCC |
| | set19 | | AGCACTGCACTTCTTGAGTT |
| SEQ ID NO: 177 | LF RSV-A- | #7 | TTGTAAGTGATGCAGGAT |
| | set19 | | |
| SEQ ID NO: 178 | LB RSV-A- | #7 | AGGGACCCTCATTAAGAGTCATG |
| | set19 | | |
| SEQ ID NO: 179 | F3 RSV-A- | #8 | ATACACAGCTGCTGTTCA |
| | set20 | | |
| SEQ ID NO: 174 | B3 RSV-A- | #8 | GGTAAATTTGCTGGGCATT |
| | set20 | | |
| SEQ ID NO: 180 | FIP RSV-A- | #8 | TCTGCTGGCATGGATGATTGAATG |
| | set20 | | TCCTAGAAAAAGACGATG |
| SEQ ID NO: 176 | BIP RSV-A- | #8 | CTAGTGAAACAAATATCCACACCC |
| | set20 | | AGCACTGCACTTCTTGAGTT |
| SEQ ID NO: 181 | LF RSV-A- | #8 | CCCATATTGTAAGTGATGCAGGAT |
| | set20 | | |
| SEQ ID NO: 182 | LB RSV-A- | #8 | AGGGACCCTCATTAAGAGTCAT |
| | set20 | | |
| SEQ ID NO: 179 | F3 RSV-A- | #9 | ATACACAGCTGCTGTTCA |
| | set21 | | |
| SEQ ID NO: 183 | B3 RSV-A- | #9 | TGGTAAATTTGCTGGGCAT |
| | set21 | | |
| SEQ ID NO: 180 | FIP RSV-A- | #9 | TCTGCTGGCATGGATGATTGAATG |
| | set21 | | TCCTAGAAAAAGACGATG |
| SEQ ID NO: 184 | BIP RSV-A- | #9 | TGAAACAAATATCCACACCCAAGG |
| | set21 | | GCACTGCACTTCTTGAGTT |
| SEQ ID NO: 185 | LF RSV-A- | #9 | CCATATTGTAAGTGATGCAGGAT |
| | set21 | | |
| SEQ ID NO: 186 | LB RSV-A- | #9 | GACCCTCATTAAGAGTCATGAT |
| | set21 | | |
| SEQ ID NO: 187 | F3 RSV-A- | #10 | AACATACGTGAACAAACTTCA |
| | set22 | | |
| SEQ ID NO: 188 | B3 RSV-A- | #10 | GCACATATGGTAAATTTGCTGG |
| | set22 | | |
| SEQ ID NO: 189 | FIP RSV-A- | #10 | ACCCATATTGTAAGTGATGCAGGA |
| | set22 | | TAGGGCTCCACATACACAG |
| SEQ ID NO: 190 | BIP RSV-A- | #10 | CTAGTGAAACAAATATCCACACCC |
| | set22 | | AAGCACTGCACTTCTTGAG |
| SEQ ID NO: 191 | LF RSV-A- | #10 | TTTCTAGGACATTGTATTGAACAG |
| | set22 | | C |
| SEQ ID NO: 192 | LB RSV-A- | #10 | GGGACCCTCATTAAGAGTCATG |
| | set22 | | |
| SEQ ID NO: 193 | IAV-MP-F3 | #1 | GACTTGAAGATGTCTTTGC |
| SEQ ID NO: 194 | IAV-MP B3 | #1 | TGTTGTTTGGGTCCCCATT |
| SEQ ID NO: 195 | IAV-MP-FIP | #1 | TTAGTCAGAGGTGACAGGATTGCA |
| | | | GATCTTGAGGCTCTC |
| SEQ ID NO: 196 | IAV-MP-BIP | #1 | TTGTGTTCACGCTCACCGTGTTTGG |
| | | | ACAAAGCGTCTACG |
| SEQ ID NO: 197 | IAV-MP FL | #1 | GTCTTGTCTTTAGCCA |
| SEQ ID NO: 198 | IAV-MP BL | #1 | CAGTGAGCGAGGACTG |
| SEQ ID NO: 199 | IAV F3 v2 | #2 | ACCGAGGTCGAAACGT |
| SEQ ID NO: 200 | IAV B3 v2 | #2 | GGTCCCCATTCCCATTG |
| SEQ ID NO: 201 | IAV FIP v2 | #2 | CAAAGACATCTTCAAGTCTCTGCG |
| | | | TTTTTTCTCTCTATCGTCCCGTCA |
| SEQ ID NO: 202 | IAV BIP v2 | #2 | AATGGCTAAAGACAAGACCAATCC |
| | | | TTTTTTGTCTACGCTGCAGTCC |
| SEQ ID NO: 203 | IAV LF v2 | #2 | CGATCTCGGCTTTGAGGG |
| SEQ ID NO: 204 | IAV LB v2 | #2 | TCACCGTGCCCAGTGAG |
| SEQ ID NO: 205 | IAV F3 v3 | #3 | CGAAAGCAGGTAGATATTGAAAG |
| SEQ ID NO: 206 | IAV B3 v3 | #3 | TCTACGCTGCAGTCCTC |
| SEQ ID NO: 207 | IAV FIP v3 | #3 | TCAAGTCTCTGCGCGATCTCTTTTT |
| | | | TGAGTCTTCTAACCGAGGT |
| SEQ ID NO: 208 | IAV BIP v3 | #3 | AGATGTCTTTGCAGGGAAAAACAC |
| | | | TTTTTTCACAAATCCTAAAATCCCC |
| | | | TTAG |
| SEQ ID NO: 209 | IAV LF v3 | #3 | GACGATAGAGAGAACGTACGTTTC |
| SEQ ID NO: 210 | IAV LB v3 | #3 | AAGACCAATCCTGTCACCTCT |
| SEQ ID NO: 211 | IAV-set4-F3 | #4 | GCGAAAGCAGGTAGATATTGA |
| SEQ ID NO: 212 | IAV-set4-B3 | #4 | CATTCCCATTGAGGGCATT |
| SEQ ID NO: 213 | IAV-set4-FIP | #4 | CTTCAAGTCTCTGCGCGATCTATG |
| | | | AGTCTTCTAACCGAGGT |
| SEQ ID NO: 214 | IAV-set4-BIP | #4 | TTGAGGCTCTCATGGAATGGCAGC |
| | | | GTGAACACAAATCCTAA |
| SEQ ID NO: 215 | IAV-set4-LF | #4 | TGACGGGACGATAGAGAGAA |
| SEQ ID NO: 216 | IAV-set4-LB | #4 | ACAAGACCAATCCTGTCACC |
| SEQ ID NO: 211 | IAV-set5-F3 | #5 | GCGAAAGCAGGTAGATATTGA |
| SEQ ID NO: 212 | IAV-set5-B3 | #5 | CATTCCCATTGAGGGCATT |
| SEQ ID NO: 217 | IAV-set5-FIP | #5 | TTCAAGTCTCTGCGCGATCTCATG |
| | | | AGTCTTCTAACCGAGGT |
| SEQ ID NO: 214 | IAV-set5-BIP | #5 | TTGAGGCTCTCATGGAATGGCAGC |
| | | | GTGAACACAAATCCTAA |
| SEQ ID NO: 215 | IAV-set5-LF | #5 | TGACGGGACGATAGAGAGAA |
| SEQ ID NO: 216 | IAV-set5-LB | #5 | ACAAGACCAATCCTGTCACC |
| SEQ ID NO: 211 | IAV-set6-F3 | #6 | GCGAAAGCAGGTAGATATTGA |
| SEQ ID NO: 218 | IAV-set6-B3 | #6 | TTGGACAAAGCGTCTACG |
| SEQ ID NO: 213 | IAV-set6-FIP | #6 | CTTCAAGTCTCTGCGCGATCTATG |
| | | | AGTCTTCTAACCGAGGT |
| SEQ ID NO: 214 | IAV-set6-BIP | #6 | TTGAGGCTCTCATGGAATGGCAGC |
| | | | GTGAACACAAATCCTAA |
| SEQ ID NO: 215 | IAV-set6-LF | #6 | TGACGGGACGATAGAGAGAA |
| SEQ ID NO: 216 | IAV-set6-LB | #6 | ACAAGACCAATCCTGTCACC |
| SEQ ID NO: 211 | IAV-set7-F3 | #7 | GCGAAAGCAGGTAGATATTGA |
| SEQ ID NO: 212 | IAV-set7-B3 | #7 | CATTCCCATTGAGGGCATT |
| SEQ ID NO: 219 | IAV-set7-FIP | #7 | AAGTCTCTGCGCGATCTCGATGAG |
| | | | TCTTCTAACCGAGGT |
| SEQ ID NO: 214 | IAV-set7-BIP | #7 | TTGAGGCTCTCATGGAATGGCAGC |
| | | | GTGAACACAAATCCTAA |
| SEQ ID NO: 215 | IAV-set7-LF | #7 | TGACGGGACGATAGAGAGAA |
| SEQ ID NO: 216 | IAV-set7-LB | #7 | ACAAGACCAATCCTGTCACC |
| SEQ ID NO: 220 | IAV-set8-F3 | #8 | TCTTCTAACCGAGGTCGAA |
| SEQ ID NO: 221 | IAV-set8-B3 | #8 | CTGCTCTGTCCATGTTGTT |
| SEQ ID NO: 222 | IAV-set8-FIP | #8 | TCAGAGGTGACAGGATTGGTCTGA |
| | | | AGATGTCTTTGCAGGGAA |
| SEQ ID NO: 223 | IAV-set8-BIP | #8 | TTGTGTTCACGCTCACCGTCATTCC |
| | | | CATTGAGGGCATT |
| SEQ ID NO: 224 | IAV-set8-LF | #8 | ATTCCATGAGAGCCTCAAGATC |
| SEQ ID NO: 225 | IAV-set8-LB | #8 | GAGGACTGCAGCGTAGAC |
| SEQ ID NO: 226 | IAV-set9-F3 | #9 | TTCTCTCTATCGTCCCGTC |
| SEQ ID NO: 221 | IAV-set9-B3 | #9 | CTGCTCTGTCCATGTTGTT |
| SEQ ID NO: 227 | IAV-set9-FIP | #9 | CCCTTAGTCAGAGGTGACAGGAAC |
| | | | ACAGATCTTGAGGCTCT |
| SEQ ID NO: 223 | IAV-set9-BIP | #9 | TTGTGTTCACGCTCACCGTCATTCC |
| | | | CATTGAGGGCATT |
| SEQ ID NO: 228 | IAV-set9-LF | #9 | GGTCTTGTCTTTAGCCATTCCA |
| SEQ ID NO: 225 | IAV-set9-LB | #9 | GAGGACTGCAGCGTAGAC |
| SEQ ID NO: 229 | IAV-set10-F3 | #10 | GTCTTCTAACCGAGGTCGA |
| SEQ ID NO: 221 | IAV-set10-B3 | #10 | CTGCTCTGTCCATGTTGTT |
| SEQ ID NO: 230 | IAV-set10-FIP | #10 | GAGGTGACAGGATTGGTCTTGTTG |
| | | | AAGATGTCTTTGCAGGG |
| SEQ ID NO: 223 | IAV-set10-BIP | #10 | TTGTGTTCACGCTCACCGTCATTCC |
| | | | CATTGAGGGCATT |
| SEQ ID NO: 224 | IAV-set10-LF | #10 | ATTCCATGAGAGCCTCAAGATC |
| SEQ ID NO: 225 | IAV-set10-LB | #10 | GAGGACTGCAGCGTAGAC |
| SEQ ID NO: 231 | IAV-set11-F3 | #11 | AAGAAGACAAGAGATATGGC |
| SEQ ID NO: 232 | IAV-set11-B3 | #11 | CAATTCGACACTAATTGATGGC |
| SEQ ID NO: 233 | IAV-set11-FIP | #11 | GTCTCCTTGCCCAATTAGCAAGCA |
| | | | TCAATGAACTGAGCA |
| SEQ ID NO: 234 | IAV-set11-BIP | #11 | GTGGTGTTGGTAATGAAACGAAGC |
| | | | TGTCTGGCTGTCAGTA |
| SEQ ID NO: 235 | IAV-set11-LF | #11 | ACATTAGCCTTCTCTCCTTT |
| SEQ ID NO: 236 | IAV-set11-LB | #11 | AACGGGACTCTAGCATACT |
| SEQ ID NO: 237 | M605 F3 IBV | IBV | AGGGACATGAACAACAAAGA |
| | LAMP | | |
| SEQ ID NO: 238 | M606 B3 IBV | IBV | CAAGTTTAGCAACAAGCCT |
| | LAMP | | |
| SEQ ID NO: 239 | M607 FIP IBV | IBV | TCAGGGACAATACATTACGCATAT |
| | LAMP | | CGATAAAGGAGGAAGTAAACACT |
| | | | CA |
| SEQ ID NO: 240 | M608 BIP IBV | IBV | TAAACGGAACATTCCTCAAACACC |
| | LAMP | | ACTCTGGTCATATGCATTC |
| SEQ ID NO: 241 | M609 LF IBV | IBV | TCAAACGGAACTTCCCTTCTTTC |
| | LAMP | | |
| SEQ ID NO: 242 | M610 LB IBV | IBV | GGATACAAGTCCTTATCAACTCTG |
| | LAMP | | C |
| SEQ ID NO: 243 | M948 F3 | HERC2 | CTTGTAATCAACATCAGGGTAA |
| | HERC2 set3 | | |
| SEQ ID NO: 244 | M949 B3 | HERC2 | AGAAACGACAAGTAGACCATT |
| | HERC2 set3 | | |
| SEQ ID NO: 245 | M950 FIP | HERC2 | CGCCTCTTGGATCAGACACATGTG |
| | HERC2 set3 | | TTAATACAAAGGTACAGGA |
| SEQ ID NO: 246 | M951 BIP | HERC2 | CACGCTATCATCATCAGGGGCTGC |
| | HERC2 set3 | | TTCAAGTGTATATAAACTCAC |
| SEQ ID NO: 247 | M952 LF | HERC2 | GAGAGCCATGAAGAACAAATTCT |
| | HERC2 set3 | | |
| SEQ ID NO: 248 | M953 LB | HERC2 | CGAGGCTTCTCTTTGTTTTTAAT |
| | HERC2 set3 | | |

A set of LAMP primers may be designed for use in combination with a DETECTR reaction to detect a single nucleotide polymorphism (SNP) in a target nucleic acid. In some embodiments, a sequence of the target nucleic acid comprising the SNP may be reverse complementary to all or a portion of the guide nucleic acid. For example, the SNP may be positioned within a sequence of the target nucleic acid that is reverse complementary to the guide RNA sequence, as illustrated in FIG. 72C. In some cases, the sequence of the target nucleic acid sequence comprising the SNP does not overlap with or is not reverse complementary to the primers or one or more of the F1, F1c, F2, F2c, F3, F3c, B1, B1c, B2, B2c, B3, B3c, LB, LBc, LF, or LFc regions shown in FIG. 71. The guide nucleic acid may be reverse complementary to a sequence of the target nucleic acid between the F1c and B1 regions, as illustrated in FIG. 72A. The guide nucleic acid may be reverse complementary to a sequence of the target nucleic acid between the B1c and F1 regions. A guide nucleic acid may be partially reverse complementary to a sequence of the target nucleic acid between the F1c region and the B1 region, for example as illustrated in FIG. 72B. A guide nucleic acid may be partially reverse complementary to a sequence of the target nucleic acid between the B1c region and the F1 region. For example, the sequence of the target nucleic acid sequence having the SNP may be reverse complementary to at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, from 5% to 100%, from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 95%, or from 95% to 100% of the guide nucleic acid. In some cases, the guide nucleic acid does not overlap with and/or is not reverse complementary to any of the plurality of primers or the F1, F1c, F2, F2c, F3, F3c, B1, B1c, B2, B2c, B3, B3c, LB, LBc, LF, or LFc regions. Exemplary sets of DETECTR gRNAs for use in a combined RT-LAMP DETECTR or LAMP-DETECTR reaction to detect the presence of a nucleic acid sequence corresponding to a respiratory syncytial virus (RSV), an influenza A virus (IAV), an influenza B virus (IAV), or a HERC2 SNP are provided in TABLE 7.

TABLE 7
Exemplary DETECTR Guide RNAs

| SEQ ID NO: | gRNA Name | Sequence |
|---|---|---|
| SEQ ID NO: 249 | gRNA #1 (R1118) | UAAUUUCUACUAAGUGUAGAUCUUAUAA |
| | | AAGAACUAGCCAA |
| SEQ ID NO: 250 | gRNA #2 (R288) | UAAUUUCUACUAAGUGUAGAUACUCAAU |
| | | UUCCUCACUUCUC |
| SEQ ID NO: 251 | R283 | UAAUUUCUACUAAGUGUAGAUUGUUCAC |
| | | GCUCACCGUGCCC |
| SEQ ID NO: 252 | R781 | UAAUUUCUACUAAGUGUAGAUGCCAUUC |
| | | CAUGAGAGCCUCA |
| SEQ ID NO: 253 | R782 | UAAUUUCUACUAAGUGUAGAUGACAAAG |
| | | CGUCUACGCUGCA |
| SEQ ID NO: 254 | IBV (R778) | UAAUUUCUACUAAGUGUAGAUCUAACAC |
| | | UCUCAGGGACAAU |
| SEQ ID NO: 255 | A SNP Position 9 | UAAUUUCUACUAAGUGUAGAUAGCAUUA |
| | (R570) | AAUGUCAAGUUCU |
| SEQ ID NO: 256 | G SNP Position 9 | UAAUUUCUACUAAGUGUAGAUAGCAUUA |
| | (R571) | AGUGUCAAGUUCU |
| SEQ ID NO: 257 | A SNP Position 14 | UAAUUUCUACUAAGUGUAGAUAUUUGAG |
| | (R1138) | CAUUAAAUGUCAA |
| SEQ ID NO: 258 | G SNP Position 14 | UAAUUUCUACUAAGUGUAGAUAUUUGAG |
| | (R1139) | CAUUAAGUGUCAA |

### Amplification and Detection of a Single Nucleotide Polymorphism Allele

A DETECTR reaction may be used to detect the presence of a specific single nucleotide polymorphism (SNP) allele in a sample. The DETECTR reaction may produce a detectable signal, as described elsewhere herein, in the presence of a target nucleic acid comprising a specific SNP allele. The DETECTR reaction may not produce a signal in the absence of the target nucleic acid or in the presence of a nucleic acid sequence that does not comprise the specific SNP allele or comprises a different SNP allele. In some cases, a DETECTR reaction may comprise a guide RNA reverse complementary to a portion of a target nucleic acid sequence comprising a specific SNP allele. The guide RNA and the target nucleic acid comprising the specific SNP allele may bind to and activate a programmable nuclease, thereby producing a detectable signal as described elsewhere herein. The guide RNA and a nucleic acid sequence that does not comprise the specific SNP allele may not bind to or activate the programmable nuclease and may not produce a detectable signal. In some cases, a target nucleic acid sequence that may or may not comprise a specific SNP allele may be amplified using, for example, a LAMP amplification reaction. In some cases, the LAMP amplification reaction may be combined with a reverse transcription reaction, a DETECTR reaction, or both. For example, the LAMP reaction may be an RT-LAMP reaction, a LAMP DETECTR reaction, or an RT-LAMP DETECTR reactions.

A DETECTR reaction, as described elsewhere herein, may produce a detectable signal specifically in the presence of a target nucleic acid sequence comprising a specific SNP allele. For example, the DETECTR reaction may produce a detectable signal in the presence of a target nucleic acid comprising a G nucleic acid at a location of a SNP but not in the presence of a nucleic acid comprising a C, a T, or an A nucleic acid at the location of the SNP. The DETECTR reaction may produce a detectable signal in the presence of a target nucleic acid comprising a T nucleic acid at a location of a SNP but not in the presence of a nucleic acid comprising a G, a C, or an A nucleic acid at the location of the SNP. The DETECTR reaction may produce a detectable signal in the presence of a target nucleic acid comprising a C nucleic acid at a location of a SNP but not in the presence of a nucleic acid comprising a G, a T, or an A nucleic acid at the location of the SNP. The DETECTR reaction may produce a detectable signal in the presence of a target nucleic acid comprising an A nucleic acid at a location of a SNP but not in the presence of a nucleic acid comprising a G, a T, or a C nucleic acid at the location of the SNP. In addition to the DETECTR reaction, the target nucleic acid having the SNP may be concurrently, sequentially, concurrently together in a sample, or sequentially together in a sample be carried out alongside LAMP or RT-LAMP. For example, the reactions can comprise LAMP and DETECTR reactions, or RT-LAMP and DETECTR reactions. Performing a DETECTR reaction in combination with a LAMP reaction may result in an increased detectable signal as compared to the DETECTR reaction in the absence of the LAMP reaction.

In some cases, the detectable signal produced in the DETECTR reaction may be higher in the presence of a target nucleic acid comprising a specific SNP allele than in the presence of a nucleic acid that does not comprise the specific SNP allele. In some cases, the DETECTR reaction may produce a detectable signal that is at least 1-fold, at least 2-fold, at least 3-fold, at least 4-fold, at least 5-fold, at least 10-fold, at least 20-fold, at least 30-fold, at least 40-fold, at least 50-fold, at least 100-fold, at least 200-fold, at least 300-fold, at last 400-fold, at least 500-fold, at least 1000-fold, at least 2000-fold, at least 3000-fold, at least 4000-fold, at least 5000-fold, at least 6000-fold, at least 7000-fold, at least 8000-fold, at least 9000-fold, at least 10000-fold, at least 50000-fold, at least 100000-fold, at least 500000-fold, or at least 1000000-fold greater in the presence of a target nucleic acid comprising a specific SNP allele than in the presence of a nucleic acid that does not comprise the specific SNP allele. In some cases, the DETECTR reaction may produce a detectable signal that is from 1-fold to 2-fold, from 2-fold to 3-fold, from 3-fold to 4-fold, from 4-fold to 5-fold, from 5-fold to 10-fold, from 10-fold to 20-fold, from 20-fold to 30-fold, from 30-fold to 40-fold, from 40-fold to 50-fold, from 50-fold to 100-fold, from 100-fold to 500-fold, from 500-fold to 1000-fold, from 1000-fold to 10,000-fold, from 10,000-fold to 100,000-fold, or from 100,000-fold to 1,000,000-fold greater in the presence of a target nucleic acid comprising a specific SNP allele than in the presence of a nucleic acid that does not comprise the specific SNP allele.

A DETECTR reaction may be used to detect the presence of a SNP allele associated with a disease or a condition in a nucleic acid sample. The DETECTR reaction may be used to detect the presence of a SNP allele associated with an increased likelihood of developing a disease or a condition in a nucleic acid sample. The DETECTR reaction may be used to detect the presence of a SNP allele associated with a phenotype in a nucleic acid sample. For example, a DETECTR reaction may be used to detect a SNP allele associated with a disease such as phenylketonuria (PKU), cystic fibrosis, sickle-cell anemia, albinism, Huntington's disease, myotonic dystrophy type 1, hypercholesterolemia, neurofibromatosis, polycystic kidney disease, hemophilia, muscular dystrophy, hypophosphatemic rickets, Rat's syndrome, or spermatogenic failure. A DETECTR reaction may be used to detect a SNP allele associated with an increased risk of cancer, for example bladder cancer, brain cancer, breast cancer, cervical cancer, colon cancer, colorectal cancer, gallbladder cancer, stomach cancer, leukemia, liver cancer, lung cancer, oral cancer, esophageal cancer, ovarian cancer, pancreatic cancer, prostate cancer, skin cancer, testicular cancer, thyroid cancer, neuroblastoma, or lymphoma. A DETECTR reaction may be used to detect a SNP allele associated with an increased risk of a disease, for example Alzheimer's disease, Parkinson's disease, amyloidosis, heterochromatosis, celiac disease, macular degeneration, or hypercholesterolemia. A DETECTR reaction may be used to detect a SNP allele associated with a phenotype, for example, eye color, hair color, height, skin color, race, alcohol flush reaction, caffeine consumption, deep sleep, genetic weight, lactose intolerance, muscle composition, saturated fat and weight, or sleep movement.

### Definitions

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, the term “comprising” and its grammatical equivalents specifies the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein the terms “individual,” “subject,” and “patient” are used interchangeably and include any member of the animal kingdom, including humans.

As used herein the term “antibody” refers to, but not limited to, a monoclonal antibody, a synthetic antibody, a polyclonal antibody, a multispecific antibody (including a bi-specific antibody), a human antibody, a humanized antibody, a chimeric antibody, a single-chain Fvs (scFv) (including bi-specific scFvs), a single chain antibody, a Fab fragment, a F(ab′) fragment, a disulfide-linked Fvs (sdFv), or an epitope-binding fragment thereof. In some cases, the antibody is an immunoglobulin molecule or an immunologically active portion of an immunoglobulin molecule. In some instances, an antibody is animal in origin including birds and mammals. Alternately, an antibody is human or a humanized monoclonal antibody.

FIG. 1 shows a schematic, which from left to right shows, Steps 1 to 4 of a workflow. Under Step 1 is “sample preparation” in an oval. Under Step 2 is “nucleic acid amplification” in an oval. Under Step 3 is “programmable nuclease reaction incubation” in a rectangle. Under Step 4 is “detection (readout)” in a rectangle.

FIG. 2 depicts at right a filtration device shaped like a syringe. At left are three samples, which from top to bottom are cheek/facial swab, urine specimen collector, and fingerprint.

FIG. 3 shows at top a schematic entitled “device 2.1-essentials elements only/no amplification”. A sample is depicted entering through P1, which is connected vertically below to V1. V1 is adjacent to V2, which is connected vertically above to P2 through which pre-complexed programmable nuclease mix is introduced. To the right of V1 is a twisted region labeled S1. To the right of S1 is an incubation and detection chamber, labeled C1. To the right of C1 is V3, which is connected vertically above to P3, which is the collection outlet. Shown in the middle of the schematic is a fluidic device entitled “device 2.2-one-chamber reaction with amplification. A sample is depicted entering through P1, which is connected vertically below to V1. V1 is adjacent to V2, which is connected vertically above to P2 through which amplification mix is introduced. V2 is adjacent to V2, which connected vertically above to P3 through which pre-complexed programmable nuclease mix is introduced. To right of V3 is a twisted region labeled S1. To the right of S1 is an incubation and detection chamber, labeled C1. To the right of C1 is V4, which is connected vertically above to P4, which is the collection outlet. Shown at bottom is another fluidic device entitled “device 2.3-two-chamber reaction with amplification”. A sample is depicted entering through P1, which is connected vertically below to V1. V1 is adjacent to V2, which is connected vertically above to P2 through which amplification mix is introduced. To the right of V2 is a twisted region labeled S1. To the right of S1 is an incubation chamber labeled C1. To the right of C1 is V3, which is connected vertically above to P3, through which pre-complexed programmable nuclease mix is introduced. To the right of V3 is another serpentine region labeled S2. To the right of S2 is an incubation and detection chamber labeled C2. To the right of C2 is V4, which is connected vertically above to P4, which is the collection outlet.

FIG. 4 shows at top is “(a) fluorescence readout” and depicts a rectangular chip substrate surface with a thin film planar heater shown as a colored in rectangular region. Above the chip is a drawing of a fluorescence excitation/detection apparatus. Shown below is a “(b) electrochemical readout”. The electrochemical readout shows two schematics. The top schematic is titled “solid-phase detection using streptavidin signal amplification”. At left is a rectangular surface depicting the top chamber surface coated with ssDNA labeled with biotin, which is shown as stars. Directly below is an electrode surface with streptavidin, which is sown as hexagons. Shown to the right of the functionalized chambers is a graph of voltage on the x-axis versus current on the y-axis, where the graph is titled “LOW”. To the right is an arrow showing introduction of a programmable nuclease, which is depicted as a pair of scissors, and which is shown to cleave the biotin off the top surface. The biotin is depicted as attached to the streptavidin. Shown further to the right is a graph of voltage on the x-axis versus current on the y-axis, where the graph is titled “HIGH”. Shown below is the second schematic titled “solid-phase detection using immobilized electroactive oligos”. Shown at the left of the schematic is a rectangular electrode surface with ssNA/Fc-NTP. The surface is functionalize with electroactive moieties depicted as tree-like structures with ferrocene shown in circles. To the right is a graph of voltage on the x-axis versus current on the y-axis and where the graph is titled “HIGH”. Further to the right is an arrow showing introduction of a programmable nuclease, which is depicted as a pair of scissors, and which is shown to cleave the Fc circles. Further to the right is a graph of voltage on the x-axis versus current on the y-axis and where the graph is titled “LOW”.

FIG. 5 shows a sample being introduced at P1, which is connected vertically below to V1. V1 is adjacent to V2, which is connected vertically above to isothermal amplification mix. To the right of V2 is a serpentine channel labeled S1. Further to the right is an incubation chamber labeled C1. To the right of C1 is V3, which is connected vertically above to P3, through which pre-complexed programmable nuclease mix is introduced. To the right of V3 is another serpentine channel labeled S2 and further to the right is another incubation chamber labeled C2. To the right of C2 is V4, which is connected vertically above to P4 through which sucrose or a colorimetric reagent is introduced. To the right of V4 is another serpentine channel labeled S3 and further to the right is a detection chamber labeled C3 To the right of C3 is V5, which is connected vertically above to P5. Below is an exploded view diagram of the C2 incubation chamber. The schematic is of a top chamber depicted as a rectangle with a label reading “top chamber surface coated with ssNA conjugated to invertase. Invertase is shown in rectangular boxes labeled “Inv”. Below the top chamber is a structure showing a bottom chamber surface with a thin-film planar heater. Further to the right is an arrow showing introduction of a programmable nuclease, which is depicted as a pair of scissors, and which is shown to cleave the Invertase. Further below is an exploded view diagram of the detection chamber labeled C3. This exploded view diagram shows at top a schematic labeled “(a) optical readout using DNS, or other compound”. At top is “(a) optical readout using DNS, or other compound” and depicts a rectangular chip substrate surface with a thin film planar heater shown as a colored in rectangular region. Above the chip is a camera, or optical sensor. At bottom is “(b) electrochemical readout (electrochemical analyzer or glucometer”, which from left to right shows an electrode surface with immobilized glucose oxidase, which is depicted as a rectangle with an oval labeled “GOx”. Above the functionalized electrode surface is a flow diagram which from left to right shows sucrose, an arrow to the right with “Inv” directly above it, and fructose+glucose at the right. To the right of the functionalized electrode surface is a graph of voltage on the x-axis versus current on the y-axis, below which is an electronic reader indicating “LOW”. Further to the right is glucose interacting with the GOx functioanlized electrode surface resoluting in H2)2+F-glucono-δ-lactone. To the right is a graph of voltage on the x-axis versus current on the y-axis, below which is an electronic reader indicating “HIGH”. Below is a key showing that an invertase-labeled oligo is depicted as a line with a rectangle labeled “Inv”. Programmable nuclease is depicted as a pair of scissors. The molecular structure of DNS is shown. Glucose oxidase is an oval labeled as GOx.

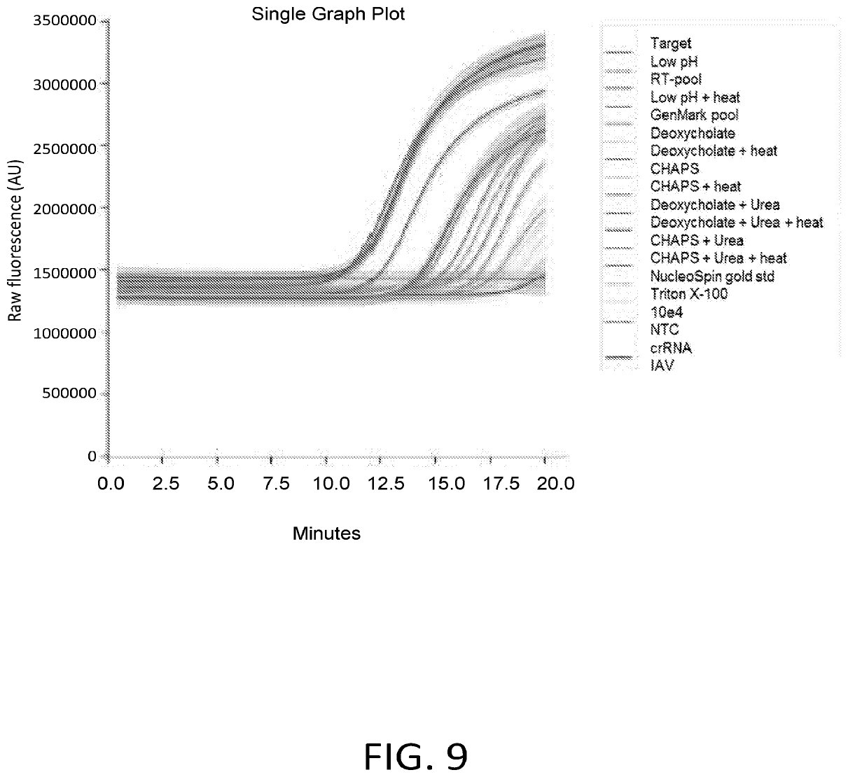

FIG. 9 depicts a line graph of raw fluorescence over time. The x-axis shows time in minutes from 0.0 to 20.0 in increments of 2.5. The y-axis shows raw fluorescence from 0 to 3,500,000 in increments of 500,000. The lines depict targets corresponding to Low pH, RT-pool, Low pH+heat, GenMark pool, Deoxycholate, Deoxycholate+heat, CHAPS, CHAPS+heat, Deoxycholate+Urea, Deoxycholate+Urea+heat, Nucleospin gold std, Triton X-100, 10e4, and NTC. The cRNA is IAV. The highest lines on the graph correspond to RT-pool, Low pH, and GenMark pool. The remaining lines, in order from upper left to lower right, correspond to NucleoSpin gold std, Doxycholate and CHAPS+Urea (approximately equal), Low pH+heat, CHAPS+heat, CHAPS+Urea+heat, Triton X-100, Deoxycholate+Urea, 10e4, and Deoxycholate+Urea+heat. NTC is a flat line at about 1,500,000.

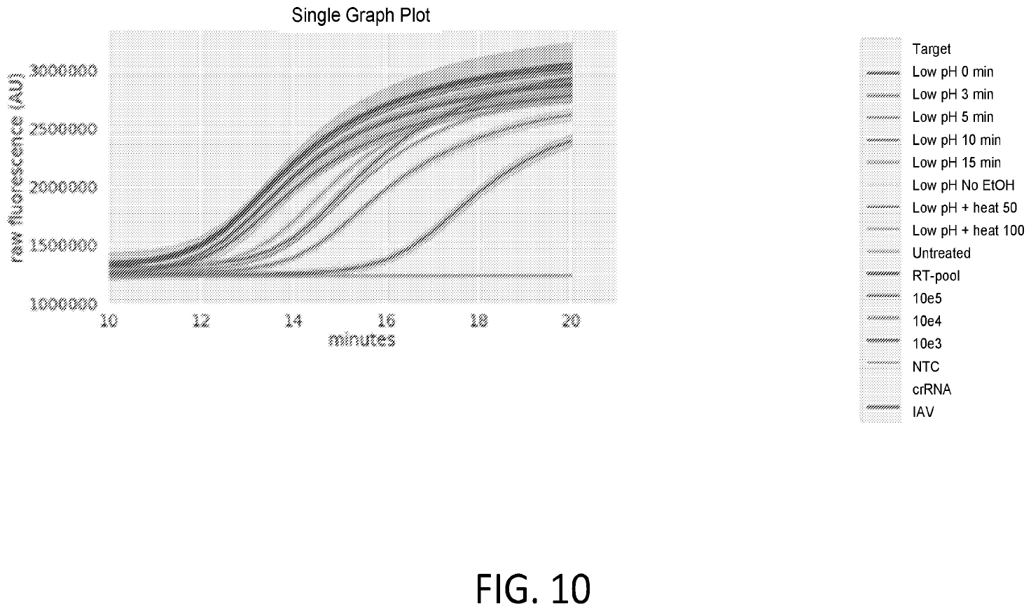

FIG. 10 depicts a line graph of raw fluorescence over time. The x-axis shows time in minutes from 0.0 to 20.0 in increments of 2.5. The y-axis shows raw fluorescence from 1,000,000 to 3,000,000 in increments of 1,000,000. The lines depict targets corresponding to Low pH 0 min, Low pH 3 min, Low pH 5 min, Low pH 10 min, Low pH 15 min, Low pH No EtOH, Low pH+heat 50, Low pH+heat 100, Untreated, RT-pool, 10e5, 10e4, 10e3, and NTC. The crRNA is IAV. The two highest lines correspond to Low pH 0 min and RT-pool. The remaining lines, from upper left to lower right, correspond to Low pH 5 min, Low pH No EtOH, Low pH 15 min, Low pH 10 min, 10e5, Low pH +heat 100, Low pH +heat 50, Untreated, 10e4, 10e3, and NTC.

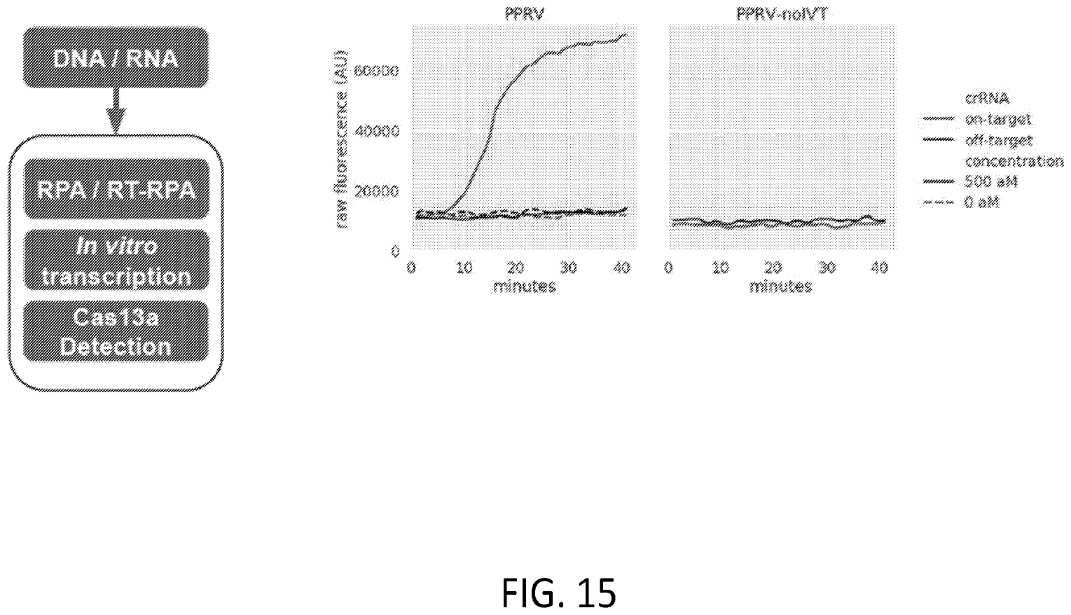

FIG. 15 depicts a flow chart and two line graphs. The flow chart shows four boxes. The top box reads “DNA/RNA.” The remaining three boxes read, from top to bottom, “RPA/RT-RPA,” “In vitro transcription,” and “Cas13a Detection.” Both plots show raw fluorescence over time. The x-axis shows minutes from 0 to 40 in increments of 10. The y-axis shows raw fluorescence (AU) from 0 to 60,000 in increments of 20,000. Both plots show two sets of two lines corresponding to on-target and off-target each at 500 aM (solid lines), and on-target and off-target each at 0 aM (dashed lines). The left plot depicts PPRV. In the left plot, the line corresponding to on-target at 500 aM rises over time. The remaining lines appear approximately flat. The right plot shows PPRV-noIVT. All four lines are approximately flat.

FIG. 17 depicts a flow chart and four line graphs. The flow chart shows four boxes. The top box reads “DNA/RNA.” The remaining three boxes read, from top to bottom, “RPA/RT-RPA,” “In vitro transcription,” and “Cas13a Detection.” All four plots show raw fluorescence over time. The x-axis of all four plots shows minutes from 0 to 20 in increments of 10. The y-axis of all four plots shows raw fluorescence (AU) from 0 to 25,000 in increments of 5,000. All four plots show two lines corresponding to crRNA on-target and off-target. The upper left plot shows +RT and +UMT. The on-target line rises over time, and the off-target line appears approximately flat. The lower left plot shows +RT and −UMT. The on-target line rises over time, and the off-target line appears approximately flat. The upper right plot shows −RT and +UMT. Both lines appear approximately flat. The lower right plot shows −RT and −UMT. Both lines appear approximately flat, but the on-target line is above the off-target line.

FIG. 20B shows a bar graph depicting time to result (lower is better). The graph shows six sets of four bars each. The six sets of bars correspond to temperatures (C) of, from left to right, of 74, 72, 70, 68, 66, and 64, as shown on the x-axis. The four bars in each set show, from left to right, Hela-total-RNA, Mouse-liver-RNA, Hela-DNA, and NTC. The y-axis shows time to result (minutes) from 0 to 40 in increments of 5. At all six temperatures, the bars corresponding to Mouse-liver-RNA and NTC have a time to result of 40 or more. At all six temperatures, Hela-total-RNA is the next highest, and Hela-DNA is the lowest.

FIG. 20C depicts three line graphs corresponding to, from left to right, crRNA=off-target, crRNA=on-target #1, and crRNA=on-target #2. For all three plots, the x-axis shows minutes from 0 to 75 in increments of 25, and the y-axis shows raw fluorescence (AU) from 0 to 1,500,000 in increments of 500,000. Each plot depicts three lines corresponding to Targets, the lines representing Hela-RNA, Hela-DNA, Mouse-liver RNA, and NTC. On the left plot and the right plot, all four lines are approximately flat. In the middle plot, the lines corresponding to Hela-DNA and Hela-RNA rise over time, with Hela-DNA being the highest. Mouse-liver-RNA and NTC are the lowest.

FIG. 21 depicts a flow chart and six line graphs. The flow chart shows three boxes labeled, from top to bottom, “DNA/RNA,” “LAMP/RT-LAMP,” and “Cas12a Detection.” The six line graphs show fluorescence over time. In all six plots, the x-axis shows minutes from 0 to 75 in increments of 25, and the y-axis shows raw fluorescence (AU) from 0 to 60,000 in increments of 20,000. All six plots show three lines corresponding to different crRNAs. The three lines show on-target #1, on-target #2, and off-target. The upper left plot shows Primers =IAV1, Target =IAV. The line corresponding to on-target #2 rises over time and is the highest. The line corresponding to off-target rises slightly over time. The line corresponding to on-target #1 appear approximately flat. The upper middle plot shows Primers=IAV2, Target=IAV. The line corresponding to on-target #2 rises over time and is the highest. The line corresponding to on-target #1 rises over time, but is not as high as on-target #2. The line corresponding to off-target rises slightly over time and is the lowest. The upper right plot shows Primers=IAV3, Target=IAV. The line corresponding to on-target #1 rises over time and is the highest. The line corresponding to off-target rises slightly over time, but is not as high as on-target #1. The line corresponding to on-target #2 appear low on the graphs and appear approximately flat. The lower left plot shows Primers=IAV1, Target=NTC. The line corresponding to on-target #2 rises over time and is the highest. The line corresponding to off-target rises slightly over time, but is not as high as on-target #2. The line corresponding to on-target #1 appears approximately flat. The lower middle plot shows Primers=IAV2, Target=NTC. The line corresponding to off target rises slightly over time. The lines corresponding to on-target #1 and on-target #2 appear approximately flat. The lower right plot shows Primers=IAV3, Target=NTC. The line corresponding to off target rises slightly over time. The lines corresponding to on-target #1 and on-target #2 appear low on the graphs and look approximately flat.

FIG. 22 depicts a flow chart and three line graphs. The flow chart shows three boxes labeled, from top to bottom, “DNA/RNA,” “LAMP/RT-LAMP,” and “Cas12a Detection.” The three line graphs show fluorescence over time. In all three plots, the x-axis shows minutes from 0 to 75 in increments of 25, and the y-axis shows raw fluorescence (AU) from 0 to 60,000 in increments of 20,000. All three plots show three lines corresponding to different crRNAs. The three lines IBV #1, IBV #2, and IBV #3. The left plot shows Target=IAV. All three lines appear approximately flat. The middle plot shows Target =IBV. The line corresponding to IBV #3 rises over time and is the highest. The line corresponding to IBV #2 rises over time, but not as rapidly as IBV #3. The line corresponding to IBV #1 appears approximately flat. The right plot shows Target=NTC. All three lines appear approximately flat.

FIG. 24B shows six line graphs. In all six plots, the x-axis shows minutes from 0 to 75 in increments of 25, and the y-axis shows raw fluorescence (AU) from 0 to 60,000 in increments of 20,000. All six plots show two lines corresponding to concentrations of 10000 and 0. The upper left plot shows Target=IAV, crRNA=IAV. The line corresponding to 10000 rises over time and is the highest. The line corresponding to 0 appear approximately flat. The upper middle plot shows Target=IBV, crRNA=IAV. Neither line is visible. The upper right plot shows Target=IAV and IBV, crRNA=IAV. The line corresponding to 10000 rises over time and is the highest. The line corresponding to 0 appears approximately flat. The lower left plot shows Target =IAV, crRNA=IBV. Neither line is visible. The lower middle plot shows Target=IBV, crRNA=IBV. The line corresponding to 10000 rises over time and is the highest. The line corresponding to 0 appear approximately flat. The lower right plot shows Target=IAV and IBV, crRNA=IBV. The line corresponding to 10000 rises over time and is the highest. The line corresponding to 0 appears approximately flat.

FIG. 25 depicts a flow chart and four line graphs. The flow chart has five boxes. The top box reads “viral RNA,” the middle box reads “multiplexed RN-LAMP,” and the remaining boxes read, from left to right, “Cas12 Influenza A detection,” “Cas12 Influenza B detection,” and “Cas12a internal amp. detection.” The four plots depict fluorescence over time. The x-axis of all four plots shows minutes from 0 to 80 in increments of 20, and the y-axis shows raw fluorescence (AU) from 0 to 50,000 in increments of 10,000. Each plot shows three lines corresponding to different crRNAs, IAV, IBV, and Mammoth IAC. The left-most plot depicts IAV. The line corresponding to IAV rises over time. The second plot from the left shows IBV. The line corresponding to IBV rises over time. The second plot from the right depicts IAV and IBV. The line corresponding to IBV rises over time and is the highest. The line corresponding to IAV rises over time, but is not as high as IBV. The right-most plot depicts IAV, IBV, and Mammoth IAC. The line corresponding to IBV rises over time and is the highest. The line corresponding to Mammoth IAC rises over time, but is not as high as IBV. The line corresponding to IAV appears approximately flat.

FIG. 49C shows six line plots depicting fluorescence over time. In all six plots the x-axis shows minutes from 0 to 75 in increments of 25, and the y-axis shows normalized fluorescence from 0.0 to 1.0 in increments of 0.2. Each plot depicts two sets of four lines. The first set of four lines shows concentrations (nM) of 2.5, 0.25, 0.025, and 0 with an RNA-FQ reporter (solid lines). The second set of four lines shows concentrations (nM) of 2.5, 0.25, 0.025, and 0 with an DNA-FQ reporter (dashed lines). The top left plot shows target=RNA, protein=Cas13M26 (LbuCas13a having a sequence of SEQ ID NO: 131). The lines corresponding to 2.5 RNA-FQ, 0.25 RNA-FQ, and 0.0025 RNA-FQ rise over time. The line corresponding to 2.5 RNA-FQ is the highest, followed by the line corresponding to 0.25 RNA-FQ, and the line corresponding to 0.025 RNA-FQ is the lowest of the three. The remaining lines are not distinguishable from the baseline. The top middle plot shows target=ssDNA, protein=Cas13M26. The lines corresponding to 2.5 RNA-FQ and 0.25 RNA-FQ rise over time. The line corresponding to 2.5 RNA-FQ is the highest, followed by the line corresponding to 0.25 RNA-FQ. The remaining lines are not distinguishable from the baseline. The top right plot shows target=dsDNA, protein=Cas13M26. None of the lines are distinguishable from baseline. The bottom left plot shows target=RNA, protein=Cas12M08 (a variant within the Cas12 family having a sequence of SEQ ID NO: 37). None of the lines are distinguishable from baseline. The bottom middle plot shows target=ssDNA, protein=Cas12M08. The lines corresponding to 2.5 DNA-FQ, 0.25 DNA-FQ, and 0.0025 DNA-FQ rise over time. The line corresponding to 2.5 DNA-FQ is the highest, followed by the line corresponding to 0.25 DNA-FQ, and the line corresponding to 0.025 DNA-FQ is the lowest of the three. The remaining lines are minimally distinguishable from the baseline. The bottom right plot shows target=dsDNA, protein=Cas12M08. The lines corresponding to 2.5 DNA-FQ, 0.25 DNA-FQ, and 0.0025 DNA-FQ rise over time. The line corresponding to 2.5 DNA-FQ is the highest, followed by the line corresponding to 0.25 DNA-FQ, and the line corresponding to 0.025 DNA-FQ is the lowest of the three. The remaining lines are minimally distinguishable from the baseline.

FIG. 50 shows two line plots depicting fluorescence over time. For both plots, the x-axis shows minutes from 0 to 50 in increments of 50, and the y-axis shows raw fluorescence (AU) from 0 to 2,000,000 in increments of 500,000. Both plots depict lines representing the reporters rep01—FAM-U5, rep08—A5, rep09—C5, rep10—G5, rep 11—T5, rep12—TA6, rep13—TA13, rep14—TA10, rep15—T6, rep16—T7, rep19—T10, rep20—T11, rep21—T12, and rep30—beacon. The left plot shows 0 nM, and none of the lines are substantially distinguishable from baseline. The right plot shows 2.5 nM. The line corresponding to rep01—FAM-up rise over time. The remaining lines are not substantially distinguishable from baseline.

FIG. 54A shows a bar plot depicting fluorescence with different crRNA and primers. The y-axis shows normalized fluorescence from 0 to 160,000 in increments of 20,000. The x-axis shows crRNA. The plot depicts two sets of three bars. The left set depcits on-target crRNA, and the right set depicts off-target crRNA. The three bars in each set correspond to the primers, from left to right, LF+LB, LF, and LB.

While various embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A microfluidic cartridge for detecting a target nucleic acid comprising: a) an amplification chamber fluidically connected to a valve; b) a detection chamber fluidically connected to the valve, wherein the valve is connected to a sample metering channel; c) a detection reagent chamber fluidically connected to the detection chamber via a resistance channel, the detection reagent chamber comprising a programmable nuclease, a guide nucleic acid, and a labeled detector nucleic acid, wherein the labeled detector nucleic acid is capable of being cleaved upon binding of the guide nucleic acid to a segment of a target nucleic acid. 2. The microfluidic cartridge of embodiment 1, wherein the sample metering channel controls volumes of liquids dispensed in a channel or chamber. 3. The microfluidic cartridge of embodiment 2, wherein the sample metering channel is fluidically connected to the detection chamber. 4. The resistance channel of any one of embodiments 1-3, wherein the resistance channel has a serpentine path, an angular path, or a circuitous path. 5. The microfluidic cartridge of any one of embodiments 1-4, wherein the valve is a rotary valve, pneumatic valve, a hydraulic valve, an elastomeric valve. 6. The microfluidic cartridge of any one of embodiments 1-5, wherein the resistance channel is fluidically connected with the valve. 7. The microfluidic cartridge of any one of embodiments 1-6, wherein the valve comprises casing, comprising a “substrate” or an “over-mold.” 8. The microfluidic cartridge of any one of embodiments 1-7, wherein the valve is actuated by a solenoid. 9. The microfluidic cartridge of any one of embodiments 1-8, wherein the valve is controlled manually, magnetically, electrically, thermally, by a bistable circuit, with a piezoelectric material, electrochemically, with phase change, rehologically, pneumatically, with a check valve, with capillarity, or any combination thereof 10. The microfluidic cartridge of any one of embodiments 5-9, wherein the rotary valve fluidically connects at least 3, at least, 4, or at least 5 chambers. 11. The microfluidic cartridge of any one of embodiments 1-10, further comprising an amplification reagent chamber fluidically connected to the amplification chamber. 12. The microfluidic cartridge of embodiment 11, further comprising a sample chamber fluidically connected to the amplification reagent chamber. 13. The microfluidic cartridge of embodiment 12, further comprising a sample inlet connected to the sample chamber. 14. The microfluidic cartridge of embodiment 13, wherein the sample inlet is sealable. 15. The microfluidic cartridge of embodiment 14, wherein the sample inlet forms a seal around the sample. 16. The microfluidic cartridge of any one of embodiments 12-15, wherein the sample chamber comprises a lysis buffer. 17. The microfluidic cartridge of any one of embodiments 12-16, further comprising a lysis buffer storage chamber fluidically connected to the sample chamber. 18. The microfluidic cartridge of embodiment 17, wherein the lysis buffer storage chamber comprises a lysis buffer. 19. The microfluidic cartridge of any one of embodiments 16-18, wherein the lysis buffer is a dual lysis/amplification buffer. 20. The microfluidic cartridge of any one of embodiments 17-19, wherein the lysis buffer storage chamber is fluidically connected to the sample chamber through a second valve. 21. The microfluidic cartridge of any one of embodiments 12-20, wherein the sample chamber is fluidically connected to the amplification chamber through the amplification reagent chamber. 22. The microfluidic cartridge any one of embodiments 12-20, wherein the sample chamber is fluidically connected to the amplification reagent chamber through the amplification chamber. 23. The microfluidic cartridge of any one of embodiments 11-22, wherein the microfluidic cartridge is configured to direct fluid bidirectionally between the amplification reagent chamber and amplification chamber. 24. The microfluidic cartridge of any one of embodiments 1-23, wherein the detection reagent chamber is fluidically connected to the amplification chamber. 25. The microfluidic cartridge of any one of embodiments 1-24, wherein the amplification chamber is fluidically connected to the detection chamber through the detection reagent chamber. 26. The microfluidic cartridge of any one of embodiments 1-25, further comprising a reagent port above the detection chamber configured to deliver fluid from the detection reagent chamber to the detection chamber. 27. The microfluidic cartridge of any one of embodiments 1-26, wherein the amplification chamber is fluidically connected to the detection reagent chamber through the detection chamber. 28. The microfluidic cartridge of any one of embodiments 1-27, wherein the resistance channel is configured to reduce backflow into the detection chamber and the detection reagent chamber. 29. The microfluidic cartridge of any one of embodiments 2-27, wherein the sample metering channel is configured to direct a predetermined volume of fluid from the detection reagent chamber to the detection chamber. 30. The microfluidic cartridge of any one of embodiments 1-29, wherein the amplification chamber and detection chamber are thermally isolated. 31. The microfluidic cartridge of any one of embodiments 1-30, wherein the detection reagent chamber is fluidically connected to the detection chamber. 32. The microfluidic cartridge of any one of embodiments 1-31, wherein the detection reagent chamber is fluidically connected to the detection chamber via a second resistance channel. 33. The microfluidic cartridge of any one of embodiments 1-32, wherein the resistance channel or the second resistance channel is a serpentine resistance channel. 34. The microfluidic cartridge of any one of embodiments 1-33, wherein the resistance channel or the second resistance channel comprises at least two hairpins. 35. The microfluidic cartridge of any one of embodiments 1-34, wherein the resistance channel or the second resistance channel comprises at least one, at least 2, at least 3, or at least 4 right angles. 36. The microfluidic cartridge of any one of embodiments 1-35, wherein the amplification chamber comprises a sealable sample inlet. 37. The microfluidic cartridge of embodiment 36, wherein the sample inlet is configured to form a seal around a swab. 38. The microfluidic cartridge of any one of embodiments 1-37, wherein microfluidic cartridge is configured to connect to a first pump to pump fluid from the amplification chamber to the detection chamber. 39. The microfluidic cartridge of any one of embodiments 1-38, wherein microfluidic cartridge is configured to connect to a second pump to pump fluid from the detection reagent chamber to the detection chamber. 40. The microfluidic cartridge of any one of embodiments 38-39, wherein first pump or the second pump is a pneumatic pump, a peristaltic pump, a hydraulic pump, or a syringe pump. 41. The microfluidic cartridge of any one of embodiments 1-40, wherein the amplification chamber is fluidically connected to a port configured to receive pneumatic pressure. 42. The microfluidic cartridge of embodiment 41, wherein the amplification chamber is fluidically connected to the port through a channel. 43. The microfluidic cartridge of any one of embodiments 11-42, wherein the amplification reagent chamber is connected to a second port configured to receive pneumatic pressure. 44. The microfluidic cartridge of embodiment 43, wherein the amplification reagent chamber is fluidically connected to the second port through a second channel. 45. The microfluidic cartridge of any one of embodiments 11-44, wherein the microfluidic cartridge is configured to connect to a third pump to pump fluid from the amplification reagent chamber to the amplification chamber. 46. The microfluidic cartridge of embodiment 45, wherein the third pump is a pneumatic pump, a peristaltic pump, a hydraulic pump, or a syringe pump. 47. The microfluidic cartridge of any one of embodiments 1-46, wherein the detection reagent chamber is connected to a port configured to receive pneumatic pressure. 48. The microfluidic cartridge of any one of embodiments 1-47, wherein the detection reagent chamber is fluidically connected to a third port through a third channel. 49. The microfluidic cartridge of any one of embodiments 1-48, wherein the microfluidic cartridge is configured to connect to a fourth pump to pump fluid from the detection reagent chamber to the detection chamber. 50. The microfluidic cartridge of embodiment 49, wherein the fourth pump is a pneumatic pump, a peristaltic pump, a hydraulic pump, or a syringe pump. 51. The microfluidic cartridge of any one of embodiments 1-50, further comprising a plurality of ports configured to couple to a gas manifold, wherein the plurality of ports is configured to receive pneumatic pressure. 52. The microfluidic cartridge of any one of embodiments 1-51, wherein any chamber of the microfluidic cartridge is connected to the plurality of ports of embodiment 50. 53. The microfluidic cartridge of any one of embodiments 1-52, wherein the valve is opened upon application of current electrical signal. 54. The microfluidic cartridge of any one of embodiments 1-53, wherein the detection reagent chamber is circular. 55. The microfluidic cartridge of any one of embodiments 1-53, wherein the detection reagent chamber is elongated. 56. The microfluidic cartridge of any one of embodiments 1-53, wherein the detection reagent chamber is hexagonal. 57. The microfluidic cartridge of any one of embodiments 2-56, wherein a region of the resistance channel is molded to direct flow in a direction perpendicular to the net flow direction. 58. The microfluidic cartridge of any one of embodiments 2-56, wherein a region of the resistance channel is molded to direct flow in a direction perpendicular to the axis defined by two ends of the resistance channel. 59. The microfluidic cartridge of any one of embodiments 2-58, wherein a region of the resistance channel is molded to direct flow along the z-axis of the microfluidic cartridge. 60. The microfluidic cartridge of any one of embodiments 1-59, wherein the valve is fluidically connected to two detection chambers via an amplification mix splitter. 61. The microfluidic cartridge of any one of embodiments 1-60, wherein the valve is fluidically connected to 3, 4, 5, 6, 7, 8, 9, or 10 detection chambers via an amplification mix splitter. 62. The microfluidic cartridge of any one of embodiments 1-61, further comprising a second valve fluidically connected to the detection reagent chamber and the detection chamber. 63. The microfluidic cartridge of any one of embodiments 1-62, wherein the detection chamber is vented with a hydrophobic PTFE vent. 64. The microfluidic cartridge of any one of embodiments 1-63, wherein the detection chamber comprises an optically transparent surface. 65. The microfluidic cartridge of any one of embodiments 1-64, wherein the amplification chamber is configured to hold from 10 μL to 500 μL of fluid. 66. The microfluidic cartridge of any one of embodiments 11-65, wherein the amplification reagent chamber is configured to hold from 10 μL to 500 μL of fluid. 67. The microfluidic cartridge of any one of embodiments 1-66, wherein the microfluidic cartridge is configured to accept from 2 μL to 100 μL of a sample comprising a nucleic acid. 68. The microfluidic cartridge of any one of embodiments 1-67, wherein the amplification reagent chamber comprises between 5 and 200 μl an amplification buffer. 69. The microfluidic cartridge of any one of embodiments 1-68, wherein the amplification chamber comprises 45 μl amplification buffer. 70. The microfluidic cartridge of any one of embodiments 1-69, wherein the detection reagent chamber stores from 5 to 200 μl of fluid containing the programmable nuclease, the guide nucleic acid, and the labeled detector nucleic acid. 71. The microfluidic cartridge of any one of embodiments 1-70, comprising 2, 3, 4, 5, 6, 7, or 8 detection chambers. 72. The microfluidic cartridge of embodiment 71, wherein the 2, 3, 4, 5, 6, 7, or 8 detection chambers are fluidically connected to a single sample chamber. 73. The microfluidic cartridge of any one of embodiments 1-72, wherein the detection chamber holds up to 100 μL, 200 μL, 300 μL, or 400 μL of fluid. 74. The microfluidic cartridge of any one of embodiments 1-73, wherein the microfluidic cartridge comprises 5-7 layers. 75. The microfluidic cartridge of any one of embodiments 1-74, wherein the cartridge comprises layers as shown in FIG. 130B. 76. The microfluidic cartridge of any one of embodiments 1-75, further comprising a sample inlet configured to adapt with a slip luer tip. 77. The microfluidic cartridge of embodiment 76, wherein the slip luer tip is adapted to fit a syringe holding a sample. 78. The microfluidic cartridge of any one of embodiments 76-77, wherein the sample inlet is capable of being hermetically sealed. 79. The microfluidic cartridge of any one of embodiments 1-78, further comprising a sliding valve. 80. The microfluidic cartridge of embodiment 79, wherein the sliding valve connects the amplification reagent chamber to the amplification chamber. 81. The microfluidic cartridge of either of embodiments 79 or 80, wherein the sliding valve connects the amplification chamber to the detection reagent chamber. 82. The microfluidic cartridge of any one of embodiments 79-81, wherein the sliding valve connects the amplification reagent chamber to the detection chamber. 83. A manifold configured to accept the microfluidic cartridge of any one of embodiments 1-82. 84. The manifold of embodiment 83, comprising a pump configured to pump fluid into the detection chamber, an illumination source configured to illuminate the detection chamber, a detector configured to detect a detectable signal produced by the labeled detector nucleic acid, and a heater configured to heat the amplification chamber. 85. The manifold of embodiment 84, further comprising a second heater configured to heat the detection chamber. 86. The manifold of any one of embodiments 84-85, wherein the illumination source is a broad spectrum light source. 87. The manifold of any one of embodiments 84-86, wherein the illumination source light produces an illumination with a bandwidth of less than 5 nm. 88. The manifold of any one of embodiments 84-87, wherein the illumination source is a light emitting diode. 89. The manifold of embodiment 88, wherein the light emitting diode produces white light, blue light, or green light. 90. The manifold of any one of embodiments 84-89, wherein the detectable signal is light. 91. The manifold of any one of embodiments 84-90, wherein the detector is a camera or a photodiode. 92. The manifold of any one of embodiments 84-91, wherein the detector has a detection bandwidth of less than 100 nm, less than 75 nm, less than 50 nm, less than 40 nm, less than 30 nm, less than 20 nm, less than 10 nm, or less than 5 nm. 93. The manifold of any one of embodiments 84-92, further comprising an optical filter configured to be between the detection chamber and the detector. 94. The microfluidic cartridge of any one of embodiments 1-93, wherein the amplification chamber comprises amplification reagents. 95. The microfluidic cartridge of any one of embodiments 11-94, wherein the amplification reagent chamber comprises amplification reagents. 96. The microfluidic cartridge of any one of embodiments 94-95, wherein the amplification reagents comprise a primer, a polymerase, dNTPs, an amplification buffer. 97. The microfluidic cartridge of any one of embodiments 1-96, wherein the amplification chamber comprises a lysis buffer. 98. The microfluidic cartridge of any one of embodiments 11-97, wherein the amplification reagent chamber comprises a lysis buffer. 99. The microfluidic cartridge of any one of embodiments 94-98, wherein the amplification reagents comprise a reverse transcriptase. 100. The microfluidic cartridge of any one of embodiments 94-99, wherein the amplification reagents comprise reagents for thermal cycling amplification. 101. The microfluidic cartridge of any one of embodiments 94-99, wherein the amplification reagents comprise reagents for isothermal amplification. 102. The microfluidic cartridge of any one of embodiments 94-101, wherein the amplification reagents comprise reagents for transcription mediated amplification (TMA), helicase dependent amplification (HDA), circular helicase dependent amplification (cHDA), strand displacement amplification (SDA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). 103. The microfluidic cartridge of any one of embodiments 94-102, wherein the amplification reagents comprise reagents for loop mediated amplification (LAMP). 104. The microfluidic cartridge of any one of embodiments 16-103, wherein the lysis buffer and the amplification buffer are a single buffer. 105. The microfluidic cartridge of any one of embodiments 16-104, wherein the lysis buffer storage chamber comprises a lysis buffer. 106. The microfluidic cartridge of any one of embodiments 16-105, wherein the lysis buffer has a pH of from pH 4 to pH 5. 107. The microfluidic cartridge of any one of embodiments 1-106, wherein the microfluidic cartridge further comprises reverse transcription reagents. 108. The microfluidic cartridge of embodiment 107, wherein the reverse transcription reagents comprise a reverse transcriptase, a primer, and dNTPs. 109. The microfluidic cartridge of any one of embodiments 1-108, wherein the programmable nuclease comprises an RuvC catalytic domain. 110. The microfluidic cartridge of any one of embodiments 1-109, wherein the programmable nuclease is a type V CRISPR/Cas effector protein. 111. The microfluidic cartridge of embodiment 110, wherein the type V CRISPR/Cas effector protein is a Cas12 protein. 112. The microfluidic cartridge of embodiment 111, wherein the Cas12 protein comprises a Cas12a polypeptide, a Cas12b polypeptide, a Cas12c polypeptide, a Cas12d polypeptide, a Cas12e polypeptide, a C2c4 polypeptide, a C2c8 polypeptide, a C2c5 polypeptide, a C2c10 polypeptide, and a C2c9 polypeptide. 113. The microfluidic cartridge of any one of embodiments 110-112, wherein the Cas12 protein has at least 80%, at least 85%, at least 90%, at least 92%, at least 95%, at least 97%, or at least 99% sequence identity to any one of SEQ ID NO: 27-SEQ ID NO: 37. 114. The microfluidic cartridge of any one of embodiments 110-113, wherein the Cas12 protein is selected from SEQ ID NO: 27-SEQ ID NO: 37. 115. The microfluidic cartridge of embodiment 110, wherein the type V CRIPSR/Cas effector protein is a Cas14 protein. 116. The microfluidic cartridge of embodiment 115, wherein the Cas14 protein comprises a Cas14a polypeptide, a Cas14b polypeptide, a Cas14c polypeptide, a Cas14d polypeptide, a Cas14e polypeptide, a Cas 14f polypeptide, a Cas14g polypeptide, a Cas14h polypeptide, a Cas14i polypeptide, a Cas14j polypeptide, or a Cas14k polypeptide. 117. The microfluidic cartridge of any one of embodiments 115-116, wherein the Cas14 protein has at least 80%, at least 85%, at least 90%, at least 92%, at least 95%, at least 97%, or at least 99% sequence identity to any one of SEQ ID NO: 38-SEQ ID NO: 129. 118. The microfluidic cartridge of any one of embodiments 115-117, wherein the Cas14 protein is selected from SEQ ID NO: 38-SEQ ID NO: 129. 119. The microfluidic cartridge of embodiment 110, wherein the type V CRIPSR/Cas effector protein is a CasΦ protein. 120. The microfluidic cartridge of embodiment 119, wherein the CasΦ protein has at least 80%, at least 85%, at least 90%, at least 92%, at least 95%, at least 97%, or at least 99% sequence identity to any one of SEQ ID NO: 274-SEQ ID NO: 321. 121. The microfluidic cartridge of any one of embodiments 119-120, wherein the CasΦ protein is selected from SEQ ID NO: 274-SEQ ID NO: 321. 122. The microfluidic cartridge of any one of embodiments 1-121, the microfluidic cartridge further providing one or more chambers for in vitro transcribing amplified coronavirus target nucleic acid. 123. The microfluidic cartridge of embodiment 122, wherein the in vitro transcribing comprises contacting the amplified coronavirus target nucleic acid to reagents for in vitro transcription. 124. The microfluidic cartridge of embodiment 123, wherein the reagents for in vitro transcription comprise an RNA polymerase, NTPs, and a primer. 125. The microfluidic cartridge of any one of embodiments 1-124, wherein the programable nuclease comprises a HEPN cleaving domain. 126. The microfluidic cartridge of any one of embodiments 1-125, wherein the programmable nuclease is a type VI CRISPR/Cas effector protein. 127. The microfluidic cartridge of embodiment 126, wherein the type VI CRISPR/Cas effector protein is a Cas13 protein. 128. The microfluidic cartridge of embodiment 127, wherein the Cas13 protein comprises a Cas13a polypeptide, a Cas13b polypeptide, a Cas13c polypeptide, a Cas13c polypeptide, a Cas13d polypeptide, or a Cas13e polypeptide. 129. The microfluidic cartridge of any one of embodiments 127-128, wherein the Cas13 protein has at least 80%, at least 85%, at least 90%, at least 92%, at least 95%, at least 97%, or at least 99% sequence identity to any one of SEQ ID NOs: 130-SEQ ID NO: 137. 130. The microfluidic cartridge of any one of embodiments 127-129, wherein the Cas13 protein is selected from SEQ ID NOs: 130-SEQ ID NO: 137. 131. The microfluidic cartridge of any one of embodiments 1-130, wherein the target nucleic acid is from a virus. 132. The microfluidic cartridge of embodiment 131, wherein the virus comprises a respiratory virus. 133. The microfluidic cartridge of embodiment 132, wherein the respiratory virus is an upper respiratory virus. 134. The microfluidic cartridge of embodiment 131, wherein the virus comprises an influenza virus. 135. The microfluidic cartridge of any one of embodiments 131-133, wherein the virus comprises a coronavirus. 136. The microfluidic cartridge of embodiment 135, wherein the coronavirus target nucleic acid is from SARS-CoV-2. 137. The microfluidic cartridge of any one of embodiments 135-136, wherein the coronavirus target nucleic acid is from an N gene, an E gene, or a combination thereof 138. The microfluidic cartridge of any one of embodiments 135-137, wherein the coronavirus target nucleic acid has a sequence of any one of SEQ ID NO: 333-SEQ ID NO: 338. 139. The microfluidic cartridge of any one of embodiments 135-138, wherein the guide nucleic acid is a guide RNA. 140. The microfluidic cartridge of any one of embodiments 135-139, wherein the guide nucleic acid has at least 80%, at least 85%, at least 90%, at least 92%, at least 95%, at least 97%, or at least 99% sequence identify to any one of SEQ ID NO: 323-SEQ ID NO: 328. 141. The microfluidic cartridge of any one of embodiments 135-140, wherein the guide nucleic acid is selected from any one of SEQ ID NO: 323-SEQ ID NO: 328. 142. The microfluidic cartridge of any one of embodiments 1-141, wherein the microfluidic cartridge comprises a control nucleic acid. 143. The microfluidic cartridge of embodiment 142, wherein the control nucleic acid is in the detection chamber. 144. The microfluidic cartridge of any one of embodiments 142-143, wherein the control nucleic acid is RNaseP. 145. The microfluidic cartridge of any one of embodiments 142-144, wherein the control nucleic acid has a sequence of SEQ ID NO: 379. 146. The microfluidic cartridge of any one of embodiments 142-144, wherein the guide nucleic acid has at least 80%, at least 85%, at least 90%, at least 92%, at least 95%, at least 97%, or at least 99% sequence identify to any one of SEQ ID NO: 330-SEQ ID NO: 332. 147. The microfluidic cartridge of any one of embodiments 142-146, wherein the guide nucleic acid is selected from any one of SEQ ID NO: 330-SEQ ID NO: 332. 148. The microfluidic cartridge of any one of embodiments 134-147, wherein the influenza virus comprises an influenza A virus, influenza B virus, or a combination thereof 149. The microfluidic cartridge of embodiment 1-148, wherein the guide nucleic acid targets a plurality of target sequences. 150. The microfluidic cartridge of embodiment 1-149, wherein the system comprises a plurality of guide sequences tiled against a virus. 151. The microfluidic cartridge of embodiment 150, wherein the plurality of target sequences comprises sequences from influenza A virus, influenza B virus, and a third pathogen. 152. The microfluidic cartridge of any one of embodiments 1-151, wherein the labeled detector nucleic acid comprises a single stranded reporter comprising a detection moiety 153. The microfluidic cartridge of embodiment 152, wherein the detection moiety is a fluorophore, a FRET pair, a fluorophore/quencher pair, or an electrochemical reporter molecule. 154. The microfluidic cartridge of embodiment 153, wherein the electrochemical reporter molecule comprises a species shown in FIG. 149. 155. The microfluidic cartridge of any one of embodiments 1-154, wherein the labeled detector produced a detectable signal upon cleavage of the detector nucleic acid. 156. The microfluidic cartridge of embodiment 155, wherein the detectable signal is a colorimetric signal, a fluorescence signal, an amperometric signal, or a potentiometric signal. 157. A method of detecting a target nucleic acid, the method comprising: a) providing a sample from a subject; b) adding the sample to the microfluidic cartridge of any one of embodiments 1-156; c) correlating the detectable signal of any one of embodiments 84-156 to the presence or absence of the target nucleic acid; and d) optionally quantifying the detectable signal, thereby quantifying an amount of the target nucleic acid present in the sample. 158. The use of a microfluidic cartridge according to any one of embodiments 1-156 in a method of detecting a target nucleic acid. 159. The use of a system according to any one of embodiments 1-156 in a method of detecting a targeting nucleic acid. 160. The use of a programmable nuclease in a method of detecting a target nucleic acid according to any one of embodiments 30-63, 66, 150, 153. 161. The use of a composition according to any one of embodiments 66-87 in a method of detecting a target a nucleic acid. 162. The use of a DNA-activated programmable RNA nuclease in a method of assaying for a target deoxyribonucleic acid from a virus in a sample according to any one of embodiments 88, 90-106 or 151. 163. The use of a DNA-activated programmable RNA nuclease in a method of assaying for a target ribonucleic acid from a virus in a sample according to any one of embodiments 88, 90-106, or 152. 164. The use of a programmable nuclease in a method of detecting a target nucleic acid in a sample according to any one embodiments 108-120, 123-148 or 156. 165. A glass capillary comprising dried reagents comprising: a) a programmable nuclease b) a guide nucleic acid c) a labeled detector nucleic acid. wherein the labeled detector nucleic acid is capable of being cleaved upon binding of the guide nucleic acid to a segment of a target nucleic acid. 166. The glass capillary of embodiment 165, wherein the dried reagents can be stored for more than 1 year. 167. The glass capillary of embodiment 165, wherein the dried reagents can be stored up to 7 months, up to 8 months, up to 9 months, up to 10 months, or up to 11 months. 168. The glass capillary of any one of embodiments 165-167, wherein the dried reagents can be stably stored at room temperature. 169. The glass capillary of any one of embodiments 165-168, wherein the dried reagents can be stably stored at 4° C. 170. The glass capillary of any one of embodiments 165-169, wherein a sample comprising the target nucleic acid can be eluted through the capillary. 171. The glass capillary of embodiment 98, wherein the sample rehydrates the dried reagents. 172. The glass capillary of any one of embodiments 165-170, wherein the glass capillary is adapted to a microfluidic cartridge of any one of embodiments 1-156 or the maniforld of any one of embodiments 83-93 for readout of a detectable signal emitted from the cleaved labeled detector nucleic acid, wherein the detectable signal is an optical signal. 173. A method of detecting a target nucleic acid, the method comprising: a) providing a sample from a subject; b) adding the sample to the glass capillary of any one of embodiments 165-172; c) correlating the detectable signal of any one of embodiments 84-173 to the presence or absence of the target nucleic acid; and d) optionally quantifying the detectable signal, thereby quantifying an amount of the target nucleic acid present in the sample. 174. A spin through column comprising a) an upper chamber comprising a programmable nuclease, a guide nucleic acid, a labeled detector nucleic acid wherein the labeled detector nucleic acid is capable of being cleaved upon binding of the guide nucleic acid to a segment of a target nucleic acid and wherein the upper chamber has a first side that is sealed and a second side comprising a filter; and b) a lower chamber comprising reagents for amplification and a sample having the target nucleic acid, wherein the lower chamber is attached to the upper chamber via the second side of the upper chamber. 175. The spin through column of embodiment 174, wherein upon centrifugation, the programmable nuclease, the guide nucleic acid, and the labeled detector nucleic acid flows through to the bottom chamber via the filter. 176. The spin through column of any one of embodiments 174-175, wherein the lower chamber can be imaged for a detectable signal from the cleaved labeled detector nucleic acid. 177. The spin through column of any one of embodiments 174-176, wherein the spin through column is capable of being hermetically sealed. 178. The spin through column of any one of embodiments 174-177, wherein the upper chamber is thermally isolated from the lower chamber. 179. A method of assaying for a segment of a coronavirus target nucleic acid in a sample, the method comprising: a) contacting the sample to: i) a detector nucleic acid; and ii) a composition comprising a programmable nuclease and a non-naturally occurring guide nucleic acid that hybridizes to a segment of the target nucleic acid, wherein the programmable nuclease the detector nucleic acid upon hybridization of the non-naturally occurring guide nucleic acid to the segment of the coronavirus target nucleic acid; and b) assaying for a change in a signal, wherein the change in the signal is produced by cleavage of the detector nucleic acid. 180. The method of embodiment 179, wherein the coronavirus target nucleic acid is from SARS-CoV-2. 181. The method of embodiment 179, wherein the coronavirus target nucleic acid is from an E gene, an N gene, or a combination thereof 182. The method of embodiment 179, wherein the coronavirus target nucleic acid has a sequence of any one of SEQ ID NO: 333-SEQ ID NO: 338. 183. The method of any one of embodiments 179-182, wherein the guide nucleic acid is a guide RNA. 184. The method of any one of embodiments 179-183, wherein the guide nucleic acid has at least 80%, at least 85%, at least 90%, at least 92%, at least 95%, at least 97%, or at least 99% sequence identify to any one of SEQ ID NO: 323-SEQ ID NO: 332. 185. The method of any one of embodiments 179-184, wherein the guide nucleic acid is selected from any one of SEQ ID NO: 323-SEQ ID NO: 332. 186. The method of any one of embodiments 179-185, the method further comprising amplifying the coronavirus target nucleic acid. 187. The method of embodiment 186, wherein the amplifying the coronavirus target nucleic acid comprises contacting the sample to reagents for amplification. 188. The method of embodiment 187, wherein the contacting the sample to regents for the amplification occurs prior to the contacting the sample to the detector nucleic acid to the detector nucleic acid and the composition. 189. The method of embodiment 187, wherein the contacting the sample to regents for the amplification occurs concurrent to the contacting the sample to the detector nucleic acid to the detector nucleic acid and the composition. 190. The method of any one of embodiments 186-189, wherein the amplifying comprises thermal cycling amplification. 191. The method of any one of embodiments 186-189, wherein the amplifying comprises isothermal amplification. 192. The method of any one of embodiments 186-191, wherein the amplifying comprises transcription mediated amplification (TMA), helicase dependent amplification (HDA), circular helicase dependent amplification (cHDA), strand displacement amplification (SDA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). 193. The method of any one of embodiments 186-192, wherein the amplifying comprises loop mediated amplification (LAMP). 194. The method of any one of embodiments 187-193, wherein the reagents for amplification comprise an amplification primer, a polymerase, and dNTPs. 195. The method of any one of embodiments 187-194, wherein the reagents for amplification comprise a FIP primer, a BIP primer, a LF primer, and a LB primer. 196. The method of any one of embodiments 194-195, wherein the amplification primers are selected from SEQ ID NO: 348-SEQ ID NO: 353 or SEQ ID NO: 356-SEQ ID NO: 359. 197. The method of any one of embodiments 179-196, wherein the method further comprises reverse transcribing the coronavirus target nucleic acid. 198. The method of 197, wherein the reverse transcribing comprises contacting the sample to reagents for reverse transcription. 199. The method of 198, wherein the reagents for reverse transcription comprise a reverse transcriptase, an oligonucleotide primer, and dNTPs 200. The method of any one of embodiments 198-199, wherein the contacting the sample to reagents for reverse transcription occurs prior to the contacting the sample to the detector nucleic acid to the detector nucleic acid and the composition, prior to the contacting the sample to the reagents for amplification, or prior to both. 201. The method of any one of embodiments 198-200, wherein the contacting the sample to reagents for reverse transcription occurs concurrent to the contacting the sample to the detector nucleic acid to the detector nucleic acid and the composition, concurrent to the contacting the sample to the reagents for amplification, or concurrent to both. 202. The method of any one of embodiments 179-201, the method further comprising assaying for a control sequence by contacting a control nucleic acid to a second detector nucleic acid and a composition comprising the programmable nuclease and a non-naturally occurring guide nucleic acid that hybridizes to a segment of the control nucleic acid, wherein the programmable nuclease the detector nucleic acid upon hybridization of the non-naturally occurring guide nucleic acid to the segment of the control nucleic acid. 203. The method of embodiment 202, wherein the control nucleic acid is RNase P. 204. The method of any one of embodiments 202-203, wherein the control nucleic acid has a sequence of SEQ ID NO: 339. 205. The method of any one of embodiments 179-204, wherein the guide nucleic acid has at least 80%, at least 85%, at least 90%, at least 92%, at least 95%, at least 97%, or at least 99% sequence identify to SEQ ID NO: 330-SEQ ID NO: 332. 206. The method of any one of embodiments 179-205, wherein the guide nucleic acid is SEQ ID NO: 330-SEQ ID NO: 332. 207. The method of any one of embodiments 179-206, wherein the method is carried out on a lateral flow strip. 208. The method of embodiment 207, wherein the lateral flow strip comprises a sample pad region, a control line, and a test line. 209. The method of embodiment 208, further comprising adding the sample to the sample pad region. 210. The method of any one of embodiments 208-209, wherein the presence or absence of an uncleaved reporter molecule is detected at the control line and the presence or absence of a cleaved reporter molecule is present at a test line. 211. The method of any one of embodiments 179-210, wherein the method is carried out in a microfluidic cartridge of any one of embodiments 1-81. 212. The method of any one of embodiments 179-211, further comprising lysing the sample. 213. The method of embodiment 212, wherein the lysing the sample comprises contacting the sample to a lysis buffer. 214. The method of embodiment 213, wherein the lysis buffer comprises a buffering agent, a pH of from pH 4 to pH 5, and a reducing agent. 215. The method of embodiment 214, wherein the reducing agent is N-Acetyl Cysteine, Dithiothreitol, or tris(2-carboxyethyl)phosphine. 216. The method of any one of embodiments 214-215, wherein the buffering agent is Tris, phosphate, or HEPES. 217. The method of any one of embodiments 213-216, wherein the lysis buffer further comprises a chelating agent. 218. The method of embodiment 217, wherein the chelating agent is EDTA or EGTA. 219. The method of any one of embodiments 213-218, wherein the lysis buffer further comprises magnesium salt. 220. The method of embodiment 219, wherein the magnesium salt is magnesium sulfate, magnesium chloride, or magnesium acetate. 221. The method of any one of embodiments 179-220, wherein the programmable nuclease comprises an RuvC catalytic domain. 222. The method of any one of embodiments 179-221, wherein the programmable nuclease is a type V CRISPR/Cas effector protein. 223. The method of embodiment 222, wherein the type V CRISPR/Cas effector protein is a Cas12 protein. 224. The method of embodiment 223, wherein the Cas12 protein comprises a Cas12a polypeptide, a Cas12b polypeptide, a Cas12c polypeptide, a Cas12d polypeptide, a Cas12e polypeptide, a C2c4 polypeptide, a C2c8 polypeptide, a C2c5 polypeptide, a C2c10 polypeptide, and a C2c9 polypeptide. 225. The method of any one of embodiments 223-224, wherein the Cas12 protein has at least 80%, at least 85%, at least 90%, at least 92%, at least 95%, at least 97%, or at least 99% sequence identity to any one of SEQ ID NO: 27-SEQ ID NO: 37. 226. The method of any one of embodiments 223-225, wherein the Cas12 protein is selected from SEQ ID NO: 27-SEQ ID NO: 37. 227. The method of embodiment 222, wherein the type V CRIPSR/Cas effector protein is a Cas14 protein. 228. The method of embodiment 227, wherein the Cas14 protein comprises a Cas14a polypeptide, a Cas14b polypeptide, a Cas14c polypeptide, a Cas14d polypeptide, a Cas14e polypeptide, a Cas14f polypeptide, a Cas14g polypeptide, a Cas14h polypeptide, a Cas14i polypeptide, a Cas14j polypeptide, or a Cas14k polypeptide. 229. The method of any one of embodiments 227-228, wherein the Cas14 protein has at least 80%, at least 85%, at least 90%, at least 92%, at least 95%, at least 97%, or at least 99% sequence identity to any one of SEQ ID NO: 38-SEQ ID NO: 129. 230. The method of any one of embodiments 227-229, wherein the Cas14 protein is selected from SEQ ID NO: 38-SEQ ID NO: 129. 231. The method of embodiment 222, wherein the type V CRIPSR/Cas effector protein is a Case protein. 232. The method of embodiment 231, wherein the Case protein has at least 80%, at least 85%, at least 90%, at least 92%, at least 95%, at least 97%, or at least 99% sequence identity to any one of SEQ ID NO: 274-SEQ ID NO: 321. 233. The method of any one of embodiments 231-232, wherein the CasΦ protein is selected from SEQ ID NO: 274-SEQ ID NO: 321. 234. The method of any one of embodiments 179-233, the method further comprising in vitro transcribing amplified coronavirus target nucleic acid. 235. The method of embodiment 234, wherein the in vitro transcribing comprises contacting the amplified coronavirus target nucleic acid to reagents for in vitro transcription. 236. The method of embodiment 235, wherein the reagents for in vitro transcription comprise an RNA polymerase, a primer, and NTPs. 237. The method of any one of embodiments 179-236, wherein the programable nuclease comprises a HEPN cleaving domain. 238. The method of any one of embodiments 179-237, wherein the programmable nuclease is a type VI CRISPR/Cas effector protein. 239. The method of embodiment 238, wherein the type VI CRISPR/Cas effector protein is a Cas13 protein. 240. The method of embodiment 239, wherein the Cas13 protein comprises a Cas13a polypeptide, a Cas13b polypeptide, a Cas13c polypeptide, a Cas13c polypeptide, a Cas13d polypeptide, or a Cas13e polypeptide. 241. The method of any one of embodiments 239-240, wherein the Cas13 protein has at least 80%, at least 85%, at least 90%, at least 92%, at least 95%, at least 97%, or at least 99% sequence identity to any one of SEQ ID NOs: 130-SEQ ID NO: 147. 242. The method of any one of embodiments 239-241, wherein the Cas13 protein is selected from SEQ ID NOs: 130-SEQ ID NO: 147. 243. The method of any one of embodiments 179-242, further comprising multiplexed detection of more than one coronavirus target nucleic acid. 244. The method of any one of embodiments 179-243, further comprising multiplexed detection of more than one coronavirus target nucleic acid and a control nucleic acid. 245. The method of any one of embodiments 243-244, wherein the multiplexed detection is carried out in a test tube, a well plate, a lateral flow strip, or a microfluidic cartridge. 246. The method of any one of embodiments 179-245, wherein sample lysis, reverse transcription, amplification, in vitro transcription, detection, or any combination thereof is carried out in a single volume. 247. The method of any one of embodiments 179-246, wherein sample lysis, reverse transcription, amplification, in vitro transcription, detection, or any combination thereof is carried out in separate volumes. 248. A composition comprising a non-naturally occurring guide nucleic acid having at least 80%, at least 85%, at least 90%, at least 92%, at least 95%, at least 97%, or at least 99% sequence identify to any one of SEQ ID NO: 323-SEQ ID NO: 332. 249. The composition of embodiment 248, wherein the guide nucleic acid is selected from any one of SEQ ID NO: 323-SEQ ID NO: 332. 250. The composition of any one of embodiments 248-249, the composition further comprising the detector nucleic acid of any one of embodiments 179-247. 251. The composition of any one of embodiments 248-250, the composition further comprising the programmable nuclease of any one of embodiments 179-247. 252. The composition of any one of embodiments 248-251, the composition further comprising the reagents for amplification of any one of embodiments 187-247. 253. The composition of any one of embodiments 248-252, the composition further comprising the reagents for reverse transcription of any one of embodiments 200-247. 254. The composition of any one of embodiments 248-253, the composition further comprising the reagents for in vitro transcription of any one of embodiments 135-254. 255. The composition of any one of embodiments 248-254, the composition further comprising the lysis buffer of any one of embodiments 213-247. 256. The composition of any one of embodiments 248-255, the composition further comprising the control nucleic acid of any one of embodiments 244-247. 257. The composition of any one of embodiments 248-256, the composition further comprising the guide nucleic acid of any one of embodiments 202-247. 258. The composition of any one of embodiments 248-257, wherein the composition is present in a lateral flow strip of any one of embodiments 245-247. 259. The composition of any one of embodiments 248-258, wherein the composition is present in a microfluidic cartridge of any one of embodiments 1-156. 260. The use of a programmable nuclease in a method of assaying for a segment of a coronavirus target nucleic acid in sample according to any one of embodiments 179-247. 261. The use of a composition according to any one of embodiments 248-268 in a method of detecting a target nucleic acid.

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A system for detecting a target nucleic acid, said system comprising: a guide nucleic acid targeting a target sequence from a virus; a programmable nuclease capable of being activated when complexed with the guide nucleic acid and the target sequence; and a reporter, wherein the reporter is capable of being cleaved by the activated nuclease, thereby generating a detectable signal. 2. The system of embodiment 1, wherein the reporter comprises a single stranded reporter comprising a detection moiety. 3. The system of embodiment 1, wherein the virus comprises an influenza virus or a coronavirus. 4. The system of embodiment 3, wherein the influenza virus comprises an influenza A virus, influenza B virus, or a combination thereof 5. The system of embodiment 1, wherein the virus comprises a respiratory virus. 6. The system of embodiment 5, wherein the respiratory virus is an upper respiratory virus. 7. The system of embodiment 1, wherein the guide nucleic acid targets a plurality of target sequences. 8. The system of embodiment 1, wherein the system comprises a plurality of guide sequences tiled against the virus. 9. The system of embodiment 7, wherein the plurality of target sequences comprises sequences from influenza A virus, influenza B virus, and a third pathogen. 10. The system of embodiment 2, wherein the single stranded reporter comprises a detection moiety at the 5′ end. 11. The system of embodiment 2, wherein the single stranded reporter comprises a biotin-dT/FAM moiety or a biotin-dT/ROX moiety. 12. The system of embodiment 2, wherein the single stranded reporter comprises a chemical functional handle at the 3′end capable of being conjugated to a substrate. 13. The system of embodiment 12, wherein the substrate is a magnetic bead. 14. The system of embodiment 12, wherein the substrate is a surface of a reaction chamber. 15. The system of embodiment 14, wherein downstream of the reaction chamber is a test line. 16. The system of embodiment 15, wherein the test line comprises a streptavidin. 17. The system of embodiment 15, wherein downstream of the test line is a flow control line. 18. The system of embodiment 17, wherein the flow control line comprises an anti-IgG antibody. 19. The system of embodiment 18, wherein the anti-IgG antibody comprises an anti-rabbit IgG antibody. 20. The system of embodiment 11, wherein the activated nuclease is capable of cleaving the single stranded reporter and releases the biotin-dT/FAM moiety or the biotin-dT/ROX moiety. 21. The system of embodiment 20, wherein the biotin-dT/FAM moiety is capable of binding the streptavidin at the test line. 22. The system of embodiment 1, wherein the reporter is an electroactive reporter. 23. The system of embodiment 22, wherein the electroactive reporter comprises biotin and methylene blue. 24. The system of embodiment 1, wherein the reporter is an enzyme-nucleic acid. 25. The system of embodiment 24, wherein the enzyme-nucleic acid is an invertase enzyme. 26. The system of embodiment 24, wherein an enzyme of the enzyme-nucleic acid is a sterically hindered enzyme. 27. The system of embodiment 24, wherein upon cleavage of a nucleic acid of the enzyme-nucleic acid, the enzyme is functional. 28. The system of embodiment 1, wherein the detectable signal is a colorimetric signal, a fluorescence signal, an amperometric signal, or a potentiometric signal. 29. A method of detecting a target nucleic acid in a sample comprising: contacting the sample with a guide nucleic acid targeting a target sequence; a programmable nuclease capable of being activated when complexed with the guide nucleic acid and the target sequence; and a reporter, wherein the reporter is capable of being cleaved by the activated nuclease, thereby generating a detectable signal. 30. The method of embodiment 29, wherein the target nucleic acid is from an exogenous pathogen. 31. The method of embodiment 30, wherein the exogenous pathogen comprises a virus. 32. The method of embodiment 31, wherein the virus comprises an influenza virus or a coronavirus. 33. The method of embodiment 32, wherein the influenza virus comprises an influenza A virus, influenza B virus, or a combination thereof 34. The method of embodiment 31, wherein the virus comprises a respiratory virus. 35. The method of embodiment 34, wherein the respiratory virus is an upper respiratory virus. 36. The method of embodiment 31, wherein the detectable signal indicates presence of the virus in the sample. 37. The method of embodiment 31, wherein the method further comprises diagnosing a subject from which the sample was taken with the virus. 38. The method of embodiment 37, wherein the subject is a human. 39. The method of embodiment 29, wherein the sample is a buccal swab, a nasal swab, or urine. 40. The method of embodiment 29, wherein the reporter comprises a single stranded reporter comprising a detection moiety. 41. The method of embodiment 29, wherein the guide nucleic acid targets a plurality of target sequences. 42. The system of embodiment 31, wherein the method comprises tiling a plurality of guide sequences against the virus. 43. The method of embodiment 42, wherein the plurality of target sequences comprises sequences from influenza A virus, influenza B virus, and a third pathogen. 44. The method of embodiment 40, wherein the single stranded reporter comprises a detection moiety at the 5′ end. 45. The method of embodiment 40, wherein the single stranded reporter comprises a biotin-dT/FAM moiety or a biotin-dT/ROX moiety. 46. The method of embodiment 40, wherein the single stranded reporter comprises a chemical functional handle at the 3′ end capable of being conjugated to a substrate. 47. The method of embodiment 46, wherein the substrate is a magnetic bead. 48. The method of embodiment 46, wherein the substrate is a surface of a reaction chamber. 49. The method of embodiment 48, wherein downstream of the reaction chamber is a test line. 50. The method of embodiment 49, wherein the test line comprises a streptavidin. 51. The method of embodiment 49, wherein downstream of the test line is a flow control line. 52. The method of embodiment 51, wherein the flow control line comprises an anti-IgG antibody. 53. The method of embodiment 52, wherein the anti-IgG antibody comprises an anti-rabbit IgG antibody. 54. The method of embodiment 45, wherein the activated nuclease is capable of cleaving the single stranded reporter and releases the biotin-dT/FAM moiety or the biotin-dT/ROX moiety. 55. The method of embodiment 54, wherein the biotin-dT/FAM moiety is capable of binding the streptavidin at the test line. 56. The method of embodiment 29, wherein the reporter is an electroactive reporter. 57. The method of embodiment 56, wherein the electroactive reporter comprises biotin and methylene blue. 58. The method of embodiment 29, wherein the reporter is an enzyme-nucleic acid. 59. The method of embodiment 58, wherein the enzyme-nucleic acid is an invertase enzyme. 60. The method of embodiment 58, wherein an enzyme of the enzyme-nucleic acid is a sterically hindered enzyme. 61. The method of embodiment 58, wherein upon cleavage of a nucleic acid of the enzyme-nucleic acid, the enzyme is functional. 62. The method of embodiment 29, wherein the detectable signal is a colorimetric signal, a fluorescence signal, an amperometric signal, or a potentiometric signal. 63. The system of embodiment 5, wherein the respiratory virus is a lower respiratory virus. 64. The method of embodiment 34, wherein the respiratory virus is a lower respiratory virus. 65. A composition comprising: a) a DNA-activated programmable RNA nuclease; and b) a guide nucleic acid comprising a segment that is reverse complementary to a segment of a target deoxyribonucleic acid, wherein the DNA-activated programmable RNA nuclease binds to the guide nucleic acid to form a complex. 66. The composition of embodiment 65, further comprising an RNA reporter. 67. The composition of any one of embodiments 65 and 66, further comprising the target deoxyribonucleic acid from a virus. 68. The composition of any one of embodiments 65-67, wherein the target deoxyribonucleic acid is an amplicon of a nucleic acid. 69. The composition of embodiment 68, wherein the nucleic acid is a deoxyribonucleic acid or a ribonucleic acid. 70. The composition of any one of embodiments 65-69, wherein the DNA-activated programmable RNA nuclease is a Type VI CRISPR/Cas enzyme. 71. The composition of any one of embodiments 65-70, wherein the DNA-activated programmable RNA nuclease is a Cas13. 72. The composition of any one of embodiments 65-71, wherein the DNA-activated programmable RNA nuclease is a Cas13a. 73. The composition of embodiment 72, wherein the Cas13a is LbuCas13a or LwaCas13a. 74. The composition of any one of embodiments 65-73, wherein the composition has a pH from pH 6.8 to pH 8.2. 75. The composition of any one of embodiments 65-74, wherein the target deoxyribonucleic acid lacks a guanine at the 3′ end. 76. The composition of any one of embodiments 65-75, wherein the target deoxyribonucleic acid is a single-stranded deoxyribonucleic acid. 77. The composition of any one of embodiments 65-76, further comprising a support medium. 78. The composition of any one of embodiments 65-77, further comprising a lateral flow assay device. 79. The composition of any one of embodiments 65-78, further comprising a device configured for fluorescence detection. 80. The composition of any one of embodiments 65-79, further comprising a second guide nucleic acid and a DNA-activated programmable DNA nuclease, wherein the second guide nucleic acid comprises a segment that is reverse complementary to a segment of a second target deoxyribonucleic acid comprising a guide nucleic acid. 81. The composition of embodiment 80, further comprising a DNA reporter. 82. The composition of any one of embodiments 80 and 81, wherein the DNA-activated programmable DNA nuclease is a Type V CRISPR/Cas enzyme. 83. The composition of any one of embodiments 81-82, wherein the DNA-activated programmable DNA nuclease is a Cas12. 84. The composition of embodiment 83, wherein the Cas12 is a Cas12a, Cas12b, Cas12c, Cas12d, or Cas12e. 85. The composition of any one of embodiments 80-82, wherein the DNA-activated programmable DNA nuclease is a Cas14. 86. The composition of embodiment 85, wherein the Cas14 is a Cas14a, Cas14b, Cas14c, Cas14d, Cas14e, Cas 14f, Cas14g, or Cas14h. 87. The composition of embodiment 82, wherein the type V CRIPSR/Cas effector protein is a Case protein. 88. The composition of embodiment 87, wherein the Case protein has at least 80%, at least 85%, at least 90%, at least 92%, at least 95%, at least 97%, or at least 99% sequence identity to any one of SEQ ID NO: 274-SEQ ID NO: 321. 89. The composition of any one of embodiments 87-88, wherein the Case protein is selected from SEQ ID NO: 274-SEQ ID NO: 321. 90. A method of assaying for a target deoxyribonucleic acid from a virus in a sample, the method comprising: contacting the sample to a complex comprising a guide nucleic acid and a DNA-activated programmable RNA nuclease, wherein the guide nucleic acid comprises a segment that is reverse complementary to a segment of the target deoxyribonucleic acid, and assaying for a signal produced by cleavage of at least some RNA reporters of a plurality of RNA reporters. 91. A method of assaying for a target ribonucleic acid from a virus in a sample, the method comprising: amplifying a nucleic acid in a sample to produce a target deoxyribonucleic acid; contacting the target deoxyribonucleic acid to a complex comprising a guide nucleic acid and a DNA-activated programmable RNA nuclease, wherein the guide nucleic acid comprises a segment that is reverse complementary to a segment of the target deoxyribonucleic acid, and assaying for a signal produced by cleavage of at least some RNA reporters of a plurality of RNA reporters. 92. The method of any one of embodiments 90 or 91, wherein the DNA-activated programmable RNA nuclease is a Type VI CRISPR nuclease. 93. The method of any one of embodiments 90-92, wherein the DNA-activated programmable RNA nuclease is a Cas13. 94. The method of embodiment 93, wherein the Cas13 is a Cas13a. 95. The method of embodiment 94, wherein the Cas13a is LbuCas13a or LwaCas13a. 96. The method of any one of embodiments 90-95, wherein cleavage of the at least some RNA reporters of the plurality of reporters occurs from pH 6.8 to pH 8.2. 97. The method of any one of embodiments 90-96, wherein the target deoxyribonucleic acid lacks a guanine at the 3′ end. 98. The method of any one of embodiments 90-97, wherein the target deoxyribonucleic acid is a single-stranded deoxyribonucleic acid. 99. The method of any one of embodiments 90-98, wherein the target deoxyribonucleic acid is an amplicon of a ribonucleic acid. 100. The method of any one of embodiments 90-99, wherein the target deoxyribonucleic acid or the ribonucleic acid is from an organism. 101. The method of embodiment 100, wherein the organism is a virus, bacteria, plant, or animal. 102. The method of any one of embodiments 90-101, wherein the target deoxyribonucleic acid is produced by a nucleic acid amplification method. 103. The method of embodiment 102, wherein the nucleic acid amplification method is isothermal amplification. 104. The method of embodiment 102, wherein the nucleic acid amplification method is thermal amplification. 105. The method of embodiment 102, wherein the nucleic acid amplification method is recombinase polymerase amplification (RPA), transcription mediated amplification (TMA), strand displacement amplification (SDA), helicase dependent amplification (HDA), loop mediated amplification (LAMP), rolling circle amplification (RCA), single primer isothermal amplification (SPIA), ligase chain reaction (LCR), simple method amplifying RNA targets (SMART), or improved multiple displacement amplification (IMDA), or nucleic acid sequence-based amplification (NASBA). 106. The method of any one of embodiments 90-105, wherein the signal is fluorescence, luminescence, colorimetric, electrochemical, enzymatic, calorimetric, optical, amperometric, or potentiometric. 107. The method of any one of embodiments 90-106, further comprising contacting the sample to a second guide nucleic acid and a DNA-activated programmable DNA nuclease, wherein the second guide nucleic acid comprises a segment that is reverse complementary to a segment of a second target deoxyribonucleic acid comprising a guide nucleic acid. 108. The method of embodiment 39, further comprising assaying for a signal produced by cleavage of at least some DNA reporters of a plurality of DNA reporters. 109. The method of any one of embodiments 107 and 108, wherein the DNA-activated programmable DNA nuclease is a Type V CRISPR nuclease. 110. The method of any one of embodiments 107-109, wherein the DNA-activated programmable DNA nuclease is a Cas12. 111. The method of embodiment 110, wherein the Cas12 is a Cas12a, Cas12b, Cas12c, Cas12d, or Cas12e. 112. The method of embodiments 107-109, wherein the DNA-activated programmable DNA nuclease is a Cas14. 113. The method of embodiment 112, wherein the Cas14 is a Cas14a, Cas14b, Cas14c, Cas14d, Cas14e, Cas14f, Cas14g, or Cas14h. 114. The method of embodiment 107-109, wherein the DNA-activated programmable DNA nuclease is a Case protein. 115. The method of embodiment 114, wherein the Case protein has at least 80%, at least 85%, at least 90%, at least 92%, at least 95%, at least 97%, or at least 99% sequence identity to any one of SEQ ID NO: 274-SEQ ID NO: 321. 116. The method of any one of embodiments 114-115, wherein the CasΦ protein is selected from SEQ ID NO: 274-SEQ ID NO: 321. 117. The method of any one of embodiments 90-116, wherein the guide nucleic acid comprises a crRNA. 118. The method of any one of embodiments 90-117, wherein the guide nucleic acid comprises a crRNA and a tracrRNA. 119. The method of any one of embodiments 90-118, wherein the signal is present prior to cleavage of the at least some RNA reporters. 120. The method of any one of embodiments 90-119, wherein the signal is absent prior to cleavage of the at least some RNA reporters. 121. The method of any one of embodiments 90-120, wherein the sample comprises blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. 122. The method of any one of embodiments 90-121, wherein the method is carried out on a support medium. 123. The method of any one of embodiments 90-122, wherein the method is carried out on a lateral flow assay device. 124. The method of any one of embodiments 90-123, wherein the method is carried out on a device configured for fluorescence detection. 125. A method of designing a plurality of primers for amplification of a target nucleic acid, the method comprising: providing a target nucleic acid, wherein a guide nucleic acid hybridizes to the target nucleic acid and wherein at least 60% of a sequence of the target nucleic acid is between an F1c region and a B1 region or between an F1 and a B1c region; and designing the plurality of primers comprising: i) a forward inner primer comprising a sequence of the F1c region 5′ of a sequence of an F2 region; ii) a backward inner primer comprising a sequence of the B1c region 5′ of a sequence of a B2 region; iii) a forward outer primer comprising a sequence of an F3 region; and iv) a backward outer primer comprising a sequence of a B3 region. 126. A method of detecting a target nucleic acid in a sample, the method comprising: contacting the sample to: a plurality of primers comprising: i) a forward inner primer comprising a sequence corresponding to an F1c region 5′ of a sequence corresponding to an F2 region; ii) a backward inner primer comprising a sequence corresponding to a B1 c region 5′ of a sequence corresponding to a B2 region; iii) a forward outer primer comprising a sequence corresponding to an F3 region; and iv) a backward outer primer comprising a sequence corresponding to a B3 region; a guide nucleic acid, wherein the guide nucleic acid hybridizes to the target nucleic acid and wherein at least 60% of a sequence of the target nucleic acid is between the F1c region and a B1 region or between an F1 region and the B1c region; a reporter; and a programmable nuclease that cleaves the reporter when complexed with the guide nucleic acid; and measuring a detectable signal produced by cleavage of the reporter, wherein the measuring provides for detection of the target nucleic acid in the sample. 127. The method of any one of embodiments 125-126, wherein the sequence between the F1c region and the B1 region or the sequence between the B1c region and the F1 region is at least 50% reverse complementary to the guide nucleic acid sequence. 128. The method of any one of embodiments 125-127, wherein the guide nucleic acid sequence is reverse complementary to no more than 50% of the forward inner primer, the backward inner primer, or a combination thereof 129. The method of any one of embodiments 125-128, wherein the guide nucleic acid does not hybridize to the forward inner primer and the backward inner primer. 130. The method of any one of embodiments 125-129, wherein a protospacer adjacent motif (PAM) or a protospacer flanking site (PFS) is 3′ of the target nucleic acid. 131. The method of any one of embodiments 125-130, wherein a protospacer adjacent motif (PAM) or a protospacer flanking site (PFS) is 3′ of the B1 region and 5′ of the F1c region or the protospacer adjacent motif (PAM) or a protospacer flanking site (PFS) is 3′ of the F1 region and 5′ of the B1c region. 132. The method of any one of claims 125-131, wherein the 3′ end of the target nucleic acid is 5′ of the 5′ end of the F3c region or the 3′ end of the target nucleic acid is 5′ of the 5′ end of the B3c region. 133. The method of any one of embodiments 125-132, wherein the 3′ end of the target nucleic acid is 5′ of the 5′ end of the F2c region or 3′ end of the target nucleic acid is 5′ of the 5′ end of the B2c region. 134. The method of any one of embodiments 125-133, wherein the target nucleic acid is between the F1c region and the B1 region and the 3′ end of the target nucleic acid is 5′ of the 3′ end of the F2c region, or wherein the target nucleic acid is between the B1c region and the F1 region and the 3′ end of the target nucleic acid is 5′ of the 3′ end of the B2c region. 135. The method of any one of embodiments 125-134, wherein the guide nucleic acid has a sequence reverse complementary to no more than 50% of the forward inner primer, the backward inner primer, the forward outer primer, the backward outer primer, or any combination thereof 136. The method of any one of embodiments 125-135, wherein the guide nucleic acid sequence does not hybridize to the forward inner primer, the backward inner primer, the forward outer primer, the backward outer primer, or any combination thereof 137. The method of any one of embodiments 125-136, wherein the guide nucleic acid sequence has a sequence reverse complementary to no more than 50% of a sequence of an F3c region, an F2c region, the F1c region, the B1c region, an B2c region, an B3c region, or any combination thereof 138. The method of any one of embodiments 125-137, wherein the guide nucleic acid sequence does not hybridize to a sequence of an F3c region, an F2c region, the F1c region, the B1c region, an B2c region, an B3c region, or any combination thereof 139. A method of designing a plurality of primer for amplification of a target nucleic acid, the method comprising: providing the target nucleic acid comprising a sequence between a B2 region and a B1 region or between an F2 region and an F1 region that hybridizes to a guide nucleic acid; and designing the plurality of primers comprising: i) a forward inner primer comprising a sequence of the F1c region 5′ of a sequence of an F2 region; ii) a backward inner primer comprising a sequence of the B1c region 5′ of a sequence of a B2 region; iii) a forward outer primer comprising a sequence of an F3 region; and iv) a backward outer primer comprising a sequence of a B3 region. 140. A method of designing a plurality of primer for amplification of a target nucleic acid, the method comprising: providing the target nucleic acid comprising a sequence between a F1c region and an F2c region or between a B1c region and a B2c region that hybridizes to a guide nucleic acid; and designing the plurality of primers comprising: i) a forward inner primer comprising a sequence of the F1c region 5′ of a sequence of an F2 region; ii) a backward inner primer comprising a sequence of the B1c region 5′ of a sequence of a B2 region; iii) a forward outer primer comprising a sequence of an F3 region; and iv) a backward outer primer comprising a sequence of a B3 region. 141. A method of detecting a target nucleic acid in a sample, the method comprising: contacting the sample to: a plurality of primers comprising: i) a forward inner primer comprising a sequence corresponding to an F 1 c region 5′ of a sequence corresponding to an F2 region; ii) a backward inner primer comprising a sequence corresponding to a B1c region 5′ of a sequence corresponding to a B2 region; iii) a forward outer primer comprising a sequence corresponding to an F3 region; and iv) a backward outer primer comprising a sequence corresponding to a B3 region; a guide nucleic acid, wherein the target nucleic acid comprises a sequence between a B2 region and a B1 region or between the F2 region and an F1 region that hybridizes to the guide nucleic acid; a reporter; and a programmable nuclease that cleaves the reporter when complexed with the guide nucleic acid; and measuring a detectable signal produced by cleavage of the reporter, wherein the measuring provides for detection of the target nucleic acid in the sample. 142. A method of detecting a target nucleic acid in a sample, the method comprising: contacting the sample to: a plurality of primers comprising: i) a forward inner primer comprising a sequence corresponding to an F1c region 5′ of a sequence corresponding to an F2 region; ii) a backward inner primer comprising a sequence corresponding to a B1c region 5′ of a sequence corresponding to a B2 region; iii) a forward outer primer comprising a sequence corresponding to an F3 region; and iv) a backward outer primer comprising a sequence corresponding to a B3 region; a guide nucleic acid, wherein the target nucleic acid comprises a sequence between the F1c region and an F2c region or between the B1c region and a B2c region that hybridizes to the guide nucleic acid; a reporter; and a programmable nuclease that cleaves the reporter when complexed with the guide nucleic acid; and measuring a detectable signal produced by cleavage of the reporter, wherein the measuring provides for detection of the target nucleic acid in the sample. 143. The method of any one embodiment 139 or embodiment 141, wherein a protospacer adjacent motif (PAM) or a protospacer flanking site (PFS) is 3′ of the B2 region and 5′ of the B1 region or the protospacer adjacent motif (PAM) or a protospacer flanking site (PFS) is 3′ of the F2 region and 5′ of the Fl region. 144. The method of any one embodiment 140 or embodiment 142, wherein a protospacer adjacent motif (PAM) or a protospacer flanking site (PFS) is 3′ of the B1c region and 5′ of the B2c region or the protospacer adjacent motif (PAM) or a protospacer flanking site (PFS) is 3′ of the F1c region and 5′ of the F2c region. 145. The method of any one of embodiments 139-144, wherein a protospacer adjacent motif (PAM) or a protospacer flanking site (PFS) is 3′ of the target nucleic acid. 146. The method of embodiment 145, wherein the PAM and the PFS are 5′ of the 5′ end of the F1c region, 5′ of the 5′ end of the B1c region, 3′ of the 3′ end of the F3 region, 3′ of the 3′ end of the B3 region, 3′ of the 3′ end of the F2 region, 3′ of the 3′ end of the B2 region, or any combination thereof 147. The method of embodiment 146, wherein the PAM and the PFS do not overlap the F2 region, the B3 region, the F1c region, the F2 region, the B1c region, the B2 region, or any combination thereof 148. The method of any one of embodiments 145-147, wherein the PAM and the PFS do not hybridize to the forward inner primer, the backward inner primer, the forward outer primer, the backward outer primer, or any combination thereof 149. The method of any one of embodiments 125-148, wherein the plurality of primers further comprises a loop forward primer. 150. The method of any one of embodiments 125-149, wherein the plurality of primers further comprises a loop backward primer. 151. The method of any one of embodiments 149-150, wherein the loop forward primer is between an F lc region and an F2c region. 152. The method of any one of embodiments 150-151, wherein the loop backward primer is between a B1 c region and a B2c region. 153. The method of any one of embodiments 125-152, wherein the target nucleic acid comprises a single nucleotide polymorphism (SNP). 154. The method of embodiment 153, wherein the single nucleotide polymorphism (SNP) comprises a HERC2 SNP. 155. The method of embodiment 153, wherein the single nucleotide polymorphism (SNP) is associated with an increased risk or decreased risk of cancer. 156. The method of any one of embodiments 126-138 or 141-155, wherein the target nucleic acid comprises a single nucleotide polymorphism (SNP), and wherein the detectable signal is higher in the presence of a guide nucleic acid that is 100% complementary to the target nucleic acid comprising the single nucleotide polymorphism (SNP) than in the presence of a guide nucleic acid that is less than 100% complementary to the target nucleic acid comprising the single nucleotide polymorphism (SNP). 157. The method of any one of embodiments 125-156, wherein the plurality of primers and the guide nucleic acid are present together in a sample comprising the target nucleic acid. 158. The method of any one of embodiments 126-138 or 141-157, wherein the contacting the sample to the plurality of primers results in amplifying the target nucleic acid. 159. The method of embodiment 158, wherein the amplifying and the contacting the sample to the guide nucleic acid occurs at the same time. 160. The method of embodiment 158, wherein the amplifying and the contacting the sample to the guide nucleic acid occur at different times. 161. The method of any one of embodiments 126-138 or 141-160, wherein the method further comprises providing a polymerase, a dATP, a dTTP, a dGTP, a dCTP, or any combination thereof 162. The method of any one of embodiments 125-161, wherein the target nucleic acid is from a virus. 163. The method of embodiment 162, wherein the virus comprises an influenza virus, respiratory syncytial virus, coronavirus, or a combination thereof 164. The method of embodiment 163, wherein the influenza virus comprises an influenza A virus, influenza B virus, or a combination thereof 165. The method of any one of embodiments 162-164, wherein the virus comprises a respiratory virus. 166. The method of embodiment 165, wherein the respiratory virus is an upper respiratory virus. 167. The system of any one of embodiments 1-28, wherein the system further comprises a forward inner primer, a backward inner primer, a forward outer primer, a backward outer primer, a loop forward primer, a loop backward primer, or any combination thereof 168. The method of any one of embodiments 29-64, the method further comprising contacting the sample with a forward inner primer, a backward inner primer, a forward outer primer, a backward outer primer, a loop forward primer, a loop backward primer, or any combination thereof 169. The method of embodiment 87, the method further comprising amplifying the target deoxyribonucleic acid with a forward inner primer, a backward inner primer, a forward outer primer, a backward outer primer, a loop forward primer, a loop backward primer, or any combination thereof 170. The method of any one of embodiments 88-125, wherein the amplifying comprises contacting the sample to a forward inner primer, a backward inner primer, a forward outer primer, a backward outer primer, a loop forward primer, a loop backward primer, or any combination thereof 171. The system or method of any one of embodiments 3, 32, or 163, wherein the coronavirus is SARS CoV-2. 172. The method of embodiment 105, wherein the nucleic acid amplification method is loop mediated amplification (LAMP).

### EXAMPLES

The following examples are illustrative and non-limiting to the scope of the devices, systems, fluidic devices, kits, and methods described herein.

### Example 1

### Testing of Influenza

A biological sample from an individual can be tested to determine whether the individual has influenza. The biological sample can be tested to detect the presence or absence of a target nucleic acid indicative of a influenza A or influenza B virus.

An individual obtains a biological sample of urine and applies the biological sample to the reagents described herein in a reagent chamber provided in a kit. The reagents comprise a guide nucleic acid targeting a nucleic acid present in and specific to the virus, a programmable nuclease, and a single stranded detector nucleic acid with a detection moiety. The biological sample has the influenza virus, and the target nucleic acid from the virus binds to the guide nucleic acid and activates the programmable nuclease to cleave the target nucleic acid and the single stranded detector nucleic acid.

After the sample and the reagents are contacted for a predetermined time, the individual can apply the reacted sample and reagents to a sample pad region on a support medium. The support medium comprises a lateral flow assay test strip encased in a protective housing with openings for the sample pad region to apply the reacted sample and reagents and for a detection region for reading the test results. The housing also has fiduciary markers, a reference color scale, and a barcode that identifies the test performed by the kit. As the reacted sample and reagents move along the lateral flow assay test strip to the detection region, the detection moiety from the cleaved single stranded detector nucleic acid binds with a capture molecule on the support medium and a detection molecule in a detection region to generate a detectable signal on the support medium. The detectable signal can be line in the detection region of the support medium. Once the test is complete, a line for a positive control marker and another line for a positive test become visible through the detection region opening.

After a predetermined amount of time after applying the reacted sample and reagents to the support medium, the individual can use a mobile device to obtain the test results. The individual can open a mobile application for reading of the test results on a mobile device with a camera and take an image of the support medium, including the detection region, barcode, reference color scale, and fiduciary markers on the housing, using the camera of the mobile device and the GUI of the mobile application. The mobile application identifies the test based on the barcode in the image, analyzes the detection region in the image with the fiduciary markers and a reference color scale on the housing in the same image based on the identification of the test with the barcode, and determines the presence or absence of the virus. The mobile application can present the results of the test to the individual. The mobile application can store the test results in the mobile application. The mobile application can communicate with a remote device and transfer the data of the test results. The test results can be viewable remotely from the remote device by another individual, including a healthcare professional.

### Example 2

### Testing of Influenza—Dipstick Method

A biological sample from an individual can be tested to determine whether the individual has influenza. The biological sample can be tested to detect the presence or absence of a target nucleic acid indicative of influenza A or influenza B.

An individual obtains a biological sample of urine and applies the biological sample to the reagents described herein in a reagent chamber provided in a kit. The reagents comprise a guide nucleic acid targeting a nucleic acid present in and specific to the virus, a programmable nuclease, and a single stranded detector nucleic acid.

After the sample and the reagents are contacted for a predetermined time, the individual can place one end of a support medium into the reagent chamber to apply the reacted sample and reagents to a sample pad region on the support medium. The support medium comprises a lateral flow assay test strip. As the reacted sample and reagents move along the test strip to the detection region, a line for a positive control marker becomes visible in the detection region. After a predetermined amount of time after applying the reacted sample and reagents to the support medium, the support medium can be placed into a protective housing with an opening for the detection region for reading the test results, fiduciary markers, a reference color scale, and a barcode that identifies the test performed by the kit.

The individual can use a mobile device to obtain the test results. The individual can open a mobile application for reading of the test results on a mobile device with a camera and take an image of the support medium, including the detection region, barcode, reference color scale, and fiduciary markers on the housing, using the camera of the mobile device and the mobile application. The mobile application identifies the test based on the barcode in the image, analyzes the detection region in the image with the fiduciary markers and a reference color scale on the housing in the same image based on the identification of the test with the barcode, and determines the presence or absence of the virus. The mobile application can present the results of the test to the individual.

### Example 3

### Testing of Influenza—In Situ Cleaving on Support Medium

A biological sample from an individual can be tested to determine whether the individual has influenza. The biological sample can be tested to detect the presence or absence of a target nucleic acid indicative of influenza A or influenza B virus.

An individual obtains a biological sample of urine and applies the biological sample to the reagents described herein on a sample pad region on a support medium provided in a kit. The reagents comprise a guide nucleic acid targeting a nucleic acid present in and specific to the virus, a programmable nuclease, and a single stranded detector nucleic acid. The support medium comprises a lateral flow assay test strip encased in a protective housing with an opening for the detection region for reading the test results, fiduciary markers, a reference color scale, and a barcode that identifies the test performed by the kit.

After the sample and the reagents are contacted for a predetermined time on the support medium, the reacted sample and reagents move along the support medium to a detection region on the support medium. The individual can optionally place a small volume of buffer to help move the reacted sample and reagents to the detection region. As the reacted sample and reagents move along the test strip to the detection region, a line for a positive control marker becomes visible in the detection region.

The individual can use a mobile device to obtain the test results. The individual can use a mobile device to obtain the test results. The mobile application identifies the test based on the barcode in the image, analyzes the detection region in the image, and determines the presence or absence of the virus. The mobile application can present the results of the test to the individual.

### Example 4

### Testing of Multiple Influenza Viruses—Multiple Lateral Flow Assay

A biological sample from an individual can be tested to determine whether the individual has one or more than one strains of influenza (e.g., influenza A, influenza B. The biological sample can be tested to detect the presence or absence of one or more of target nucleic acids, where the individual target nucleic acid is indicative of a virus.

An individual obtains a biological sample of urine and applies the biological sample to multiple reagent chambers provided in a kit to test for a panel of influenza virus strains. Each reagents chamber comprise the reagents specific to detect one influenza virus strain. The reagents in each reagent chamber comprise a guide nucleic acid targeting a nucleic acid present in the virus; a programmable nuclease; and a single stranded detector nucleic acid.

After the sample and the reagents are contacted for a predetermined time, the individual can apply the reacted sample and reagents from one of the reagent chambers to a matched sample pad region on a support medium. Each reagent chamber has a matching sample pad region on the support medium. The support medium comprises multiple lateral flow assay test strips encased in a protective housing with openings for the matched sample pad regions to apply the reacted sample and reagents from the matching reagent chamber and for a detection region for reading the test results. The housing also has fiduciary markers, a reference color scale, and a barcode that identifies the tests performed by the kit. As the reacted sample and reagents move along the lateral flow assay test strip to the detection region, a positive control marker for each lateral flow test strip becomes visible through the detection region opening.

After a predetermined amount of time after applying the reacted sample and reagents to the support medium, the individual can use a mobile device to obtain the test results. The individual can use a mobile device to obtain the test results. The individual can use a mobile device to obtain the test results. The mobile application identifies the test based on the barcode in the image, analyzes the detection region in the image, and determines the presence or absence of the virus (influenza A, influenza B, or influenza A and B). The mobile application can present the results of the test to the individual.

### Example 5

### Testing of Multiple Strains of Influenza—Multiplexed Lateral Flow Assay

A biological sample from an individual can be tested to determine whether the individual has one or more than one strains of influenza (e.g., influenza A, influenza B. The biological sample can be tested to detect the presence or absence of one or more of target nucleic acids, where the individual target nucleic acid is indicative of a virus.

An individual obtains a biological sample of urine and applies the biological sample to a reagent chamber provided in a kit to test for a panel of influenza virus strains. The reagents chamber comprises multiple sets of reagents to detect multiple influenza virus strains. One set of reagents to detect one influenza strain comprise a guide nucleic acid targeting a nucleic acid present in and specific to the virus; a programmable nuclease; and a single stranded detector nucleic acid.

After the sample and the reagents are contacted for a predetermined time, the individual can apply the reacted sample and reagents to a sample pad region on a support medium. The support medium comprises a multiplexed lateral flow assay test strip that can detect multiple detector molecules on the test strip. The lateral flow assay strip encased in a protective housing with openings for the sample pad region to apply the reacted sample and reagents and for a detection region for reading the test results. The housing also has fiduciary markers, a reference color scale, and a barcode that identifies the tests performed by the kit. As the reacted sample and reagents move along the lateral flow assay test strip to the detection region, a positive control marker for each lateral flow test strip becomes visible through the detection region opening.

After a predetermined amount of time after applying the reacted sample and reagents to the support medium, the individual can use a mobile device to obtain the test results. The individual can use a mobile device to obtain the test results. The individual can use a mobile device to obtain the test results. The mobile application identifies the test based on the barcode in the image, analyzes the detection region in the image, and determines the presence or absence of the virus. The mobile application can present the results of the test to the individual.

### Example 6

### Detection of a Nucleic Acid from a Respiratory Virus Using a Fluidic Device

This example illustrates detection of a nucleic acid from a respiratory virus (e.g., an influenza virus) using a fluidic device. A CRISPR-Cas reaction for detection of a target nucleic acid from a respiratory virus (e.g., an influenza virus) in a sample is carried out using a fluidic device.

FIG. 1 shows a schematic illustrating a workflow of the CRISPR-Cas reaction. Step 1 in the workflow is sample preparation, Step 2 in the workflow is amplification of a target nucleic acid from a respiratory virus (e.g., an influenza virus). Step 3 in the workflow is Cas reaction incubation. Step 4 in the workflow is detection (readout). Steps 1 and 2 are optional, and steps 3 and 4 can occur concurrently, if detection and readout are incorporated to the Cas reaction. FIG. 2 shows a fluidic device for sample preparation that is used. The sample preparation fluidic device processes different types of biological sample. The biological sample is finger-prick blood, urine or swabs with fecal, nasal swab, cheek swab or other collection. The sample is prepared in a fluidic device of FIG. 2 and is then introduced into a fluidic device.

The fluidic device is one of the three fluidic devices of FIG. 3 or the fluidic device of FIG. 5. The three fluidic devices of FIG. 3 carry out a Cas reaction with a fluorescence or electrochemical readout. An exploded view diagram summarizing the fluorescence and electrochemical processes that are used for detection of the reaction are shown in FIG. 4. FIG. 4 shows schematic diagrams of a readout process that are used including (a) fluorescence readout and (b) electrochemical readout. FIG. 5 shows a fluidic device for coupled invertase/Cas reactions with colorimetric or electrochemical/glucometer readout. This diagram illustrates a fluidic device for miniaturizing a Cas reaction coupled with the enzyme invertase. Surface modification and readout processes are depicted in exploded view schemes at the bottom including (a) optical readout using DNS, or other compound and (b) electrochemical readout (electrochemical analyzer or glucometer).

A sample containing the target nucleic acid of interest from a respiratory virus (e.g., an influenza virus) is introduced into a fluidic device of FIG. 2. The sample is filtered and introduced into a fluidic device of FIG. 3 or FIG. 5, wherein the nucleic acid of interest is, optionally, amplified, and incubated with pre-complexed Cas mix. The Cas-gRNA complex binds to its matching nucleic acid target from the amplified sample and is activated into a non-specific nuclease, which cleaves a nucleic acid-based reporter molecule to generate a signal readout. A target nucleic acid of interest from a respiratory virus (e.g., an influenza virus) is detected using a fluorescence readout, an electrochemical readout, or an electrochemiluminescence readout, as shown in FIG. 4 or an optical readout or electrochemical readout, as shown in FIG. 5.

### Example 7

### Electrochemical Detection of Target Nucleic Acids from a Respiratory Virus via DETECTR Reactions using CRISPR/Cas Systems

This example describes electrochemical detection of target nucleic acids from a respiratory virus (e.g., an influenza virus) in DETECTR reactions using CRISPR/Cas systems. In this assay a biotin-streptavidin signal enhancement method is employed using a biotinylated CRISPR-Cas reporter molecule, which is cleaved by the enzyme in the presence of a positive DETECTR reaction (one in which the target nucleic acid is present).

Electrochemical detection is tested as an alternative to (1) use of ferrocene-labelled oligos immobilized on the electrode surface and (2) coupling of DETECTR to an invertase catalyzed reaction, also disclosed herein. The latter reaction produces glucose that be detected with a glucometer directly, or indirectly. Electrochemical detection and detection using ferrocene-labelled oligos are both potentiometric, while invertase catalyzed reactions are amperometric.

The reporter is cleaved using a DNAse enzyme and cleavage results in an increase in current at an oxidation peak compared to when the reporter was intact. Results are collected using a benchtop, gold-standard electrochemical analyzer (uSTAT, Metrohm, USA). The sequence of the reporter is /5Biosg/TTTTTTTTTTTTTTTTTTTT/3MeBlN/ (SEQ ID NO: 373). A cyclic voltammogram is obtained, wherein cleavage of the electroactive reporter leads to an increase in current.

### Example 8

### Fluorescence-Based Device for Detection of Target Nucleic Acids from a Respiratory Virus via DETECTR Reactions Using CRISPR-Cas Systems

This example describes a fluorescence-based device for detection of target nucleic acids from a respiratory virus (e.g., an influenza virus) in DETECTR reactions using CRISPR-Cas systems. Two approaches are used to develop a miniaturized device for DETECTR reactions for detection of target nucleic acids from a respiratory virus (e.g., an influenza virus). First, a glass capillary (Drummond Scientific, USA) is used as a single, capillarity driven vessel of the DETECTR reaction. Both flash-dried and liquid formulations of the reagents are used. Second, a commercially-available, plastic (TOPAS) microfluidic chip (Microfluidic Chip Shop, Germany) with no mechanical actuation for mixing or reagent delivery is used.

Results are collected using (1) a portable, photodiode-based fluorescence sensor (ESELog, Quiagen Lake Constance, Germany) and (2) a commercially-available transilluminator (E-GEL, Thermofisher, USA). The major advancements of the detection capabilities of this system includes an on-chip DETECR reaction. Real-time measurement of fluorescence from a one-pot reverse transcription-recombinase polymerase amplification-in vitro transcription (RT-RPA-IVT)-DETECTR reaction is carried out on chip

### Example 9

### Guide Pooling for High Sensitivity and Broad Spectrum Detection of Target Nucleic Acids from a Respiratory Virus

This example describes guide pooling for high sensitivity and broad spectrum detection of target nucleic acids from a respiratory virus (e.g., an influenza virus). While traditional detection requires one gRNA per target sequence/organism, the guide pooling methods disclosed herein, including use of multiple gRNAs with one or more CRISPR effector proteins, is amenable for both improving sensitivity (in the case of guide tiling across a single target sequence/organism) and broad spectrum detection (in the case of detecting multiple target sequences/organisms in a single reaction). Thus, guide pooling is a useful method for enhancing detection sensitivity performance as well as functioning as an initial triage step that is rapid and low-cost (e.g., an “alert” for blood-borne pathogens, pandemic flu, pan-bacterial detection, etc.) as compared to traditional diagnostic methods.

To perform a DETECTR assay, a guide RNA (crRNA) is first complexed to the Cas protein. The complexing reaction is carried out at 37° C. for 30 minutes. Reporter and additional buffer are then added to complete the complex master mix. Finally, the complex is added to the samples to detect sequences specifically targeted by the guide. By pooling multiple guide RNAs designed to target difference sequences or different sequence segments of the same target, it is possible to broaden the detection spectrum in a single reaction and increase the detection efficiency. To achieve this, guide RNAs are individually complexed to Cas protein at high concentration. Multiple guide-protein complex reactions are pooled. After pooling, the reporter and addition buffer are added to complete the pooled complexes for use in the DETECTR assay.

A. Guide Pooling for Detection of Influenza Strains

Methods for guide pooling for detection of influenza strains involved using guide RNAs for different influenza strains (e.g., strains from IAV and/or IBV). Multiple guide RNAs (e.g., 15-20) are designed to target lthe influenza strains.

The programmable nuclease used in the DETECTR assay is LbCas12a (SEQ ID NO: 27). The reporter is an 8-mer ssDNA with a FAM-labeled 5′ end and an Iowa Black FQ-labeled 3′ end.

Guide RNA (crRNA) are individually complexed with LbCas12a protein at high concentration. Each crRNA is mixed with LbCas12a in 1× MBuffer 2 (20 mM Tris HCl, pH8, 100 mM NaCl, 5 mM MgCl2, 1 mM DTT, 5% glycerol, 50 ug/mL Heparin). The concentrations of the crRNA and protein are at least 4-fold higher than those in the standard single guide complexing reaction. The mixture is incubated at 37° C. for 30 minutes to form the guide-protein complex. TABLE 8 below lists the formulation of the complexing reaction. The volumes are for one DETECTR reaction, and can be scaled up accordingly.

TABLE 8

| Component | Volume (μL) |
|---|---|
| 5X MBuffer 2 | 1 |
| crRNA (20 μM) | 0.8 |
| LbCas12a (5 μM) | 3.2 |
| TOTAL | 5 |

Guide Pooling. At the completion of the incubation, guide pools are generated by combining equal volume of individual guide-protein complexing reactions. Several pools at different n-plex (n=number of different guides) levels are generated.

5 ul of the complexing reaction is used in each 20 ul DETECTR assays. The effective concentration in the assay of guides and protein is one fourth of those in the complexing reactions.

Complex Master Mix. Complex Master Mixes of the guide pools are completed by adding equal volume of Mix2 (containing the ssDNA reporter and additional buffer, the formulation is listed in TABLE 9).

TABLE 9

| Component | Volume (μL) |
|---|---|
| H2O | 0.8 |
| ssDNA reporter (10 μM) | 0.2 |
| 5x MBuffer 2 | 3 |
| Glycerol 80% | 1 |
| Total | 5 |

Additionally, Mix2 is added to individual guide-protein complexing reaction to generate single guide complex master mix. In the complex master mix, the concentrations of the guides and proteins are diluted in half

DETECTR assay. The targets are diluted to 200 pM and 20 pM. In each DETECTR assay, 10 ul of complex master mix is mixed with 10 ul of sample in a well of a 384-well plate. The effective concentrations of the guides and protein are one fourth of those in the complexing reaction. The reaction is carried out in a TECAN Infinite 200 pro plate reader at 37 C. The fluorescence raw data file is analyzed using internal software. The kinetics of the DETECTR assay is measured by max rate (estimated rate of cleavage of the reporter by the activated Cas protein). The activity of the guide pools versus the single guide is measured against 200 pM targets (100 pM targets in the final reaction).

Signals are clearly boosted by guide pooling. For example, the signal increases as the n-plex level is increased to 10- and 20-plex and the detection sensitivity is improved from the single guide detection. The guide pools can be adjusted to detect difference targets.

Using guide pooling, detection of 10 pM targets, which is near the detection limit of the single guide assay, is improved and the pooling of the guides improves the sensitivity of the assay.

B. Guide pooling Top-Performing Individual gRNAs to Increase Assay Sensitivity for Detection of RSV

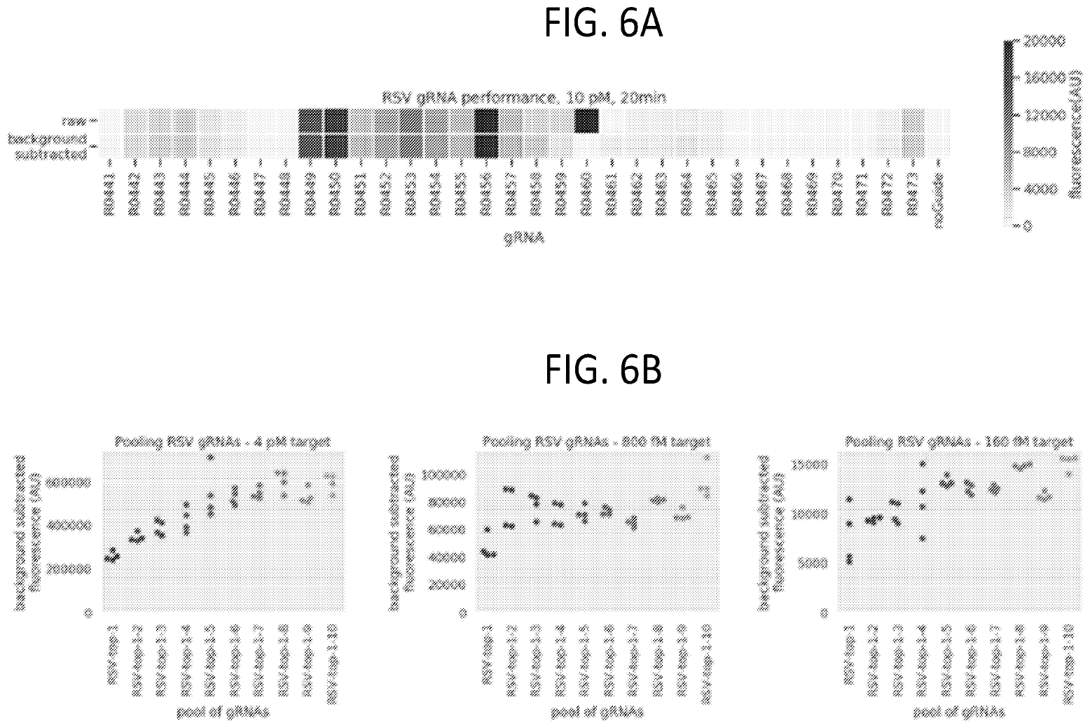

Guide pooling was used to improve the detection limit of an assay for RSV detection. 33 guide RNAs for RSV guides were designed by tiling across the target region. The guide RNAs were screened for activities and top performing guides were selected for pooling. RNA corresponding to the RSV target was generated from in vitro transcription (IVT) reaction. A Cas13a protein was used and the reporter was a 5-mer ssRNA with a 5′ FAM and a 3′ Iowa Black FQ. FIG. 6A shows a panel of gRNAs for RSV evaluated for detection efficiency. Darker squares in the background subtracted row indicate greater efficiency of detecting RSV target nucleic acids. FIG. 6B shows graphs of pools of gRNA versus background subtracted fluorescence. The left most graph shows pooling of RSV gRNAs for detection of 4 pM of target nucleic acids. The middle graph shows pooling of RSV gRNAs for detection of 800 fM of target nucleic acids. The right most graph shows pooling of RSV gRNAs for detection of 160 fM of target nucleic acids. gRNA sequences used in the RSV study are summarized below in TABLE 10.

TABLE 10

| Guide name | SEQ ID NO | crRNA Sequence |
|---|---|---|
| R0443 | SEQ ID NO: 18 | GCCACCCCAAAAAUGAAGGGGACUAAAACAUCC |
| | | UACAAAAAAAUGCUAAA |
| R0444 | SEQ ID NO: 19 | GCCACCCCAAAAAUGAAGGGGACUAAAACACCU |
| | | ACAAAAAAAUGCUAAAA |
| R0445 | SEQ ID NO: 20 | GCCACCCCAAAAAUGAAGGGGACUAAAACACUA |
| | | CAAAAAAAUGCUAAAAG |
| R0449 | SEQ ID NO: 21 | GCCACCCCAAAAAUGAAGGGGACUAAAACAAGA |
| | | AACAUUUGAUAACAAUG |
| R0450 | SEQ ID NO: 22 | GCCACCCCAAAAAUGAAGGGGACUAAAACAGAA |
| | | ACAUUUGAUAACAAUGA |
| R0452 | SEQ ID NO: 23 | GCCACCCCAAAAAUGAAGGGGACUAAAACAAAC |
| | | AUUUGAUAACAAUGAAG |
| R0453 | SEQ ID NO: 24 | GCCACCCCAAAAAUGAAGGGGACUAAAACAACA |
| | | UUUGAUAACAAUGAAGA |
| R0456 | SEQ ID NO: 25 | GCCACCCCAAAAAUGAAGGGGACUAAAACAUGC |
| | | CUAUAACAAAUGAUCAG |
| R0457 | SEQ ID NO: 26 | GCCACCCCAAAAAUGAAGGGGACUAAAACAGCC |
| | | UAUAACAAAUGAUCAGA |

### Example 10

### Optimization of Temperature and Temperature Tolerance of CRISPR-Cas Proteins in CRISPR DETECTR Assays for Detection of Target Nucleic Acids from Respiratory Viruses

This example describes optimization of temperature and temperature tolerance of CRISPR-Cas proteins in CRISPR DETECTR assays for detection of target nucleic acids from respiratory viruses (e.g., influenza virus). The CRISPR diagnostics of the present disclosure leverage the unique biochemical properties of Type V (e.g., Cas12) and Type VI (e.g., Cas13) CRISPR-Cas proteins to enable the specific detection of nucleic acids. These proteins are directed to bind a target nucleic acid from a respiratory virus (e.g., an influenza virus) by a CRISPR RNA (crRNA), which is also known as a guide RNA (gRNA). Once bound to a complementary target sequence, the Cas protein initiates indiscriminate cleavage of surrounding single-strand DNA or single-strand RNA. When coupled to a quenched fluorescence reporter or other cleavage reporter, fluorescent or other signal is generated by the Cas protein only in the presence of the target nucleic acid. CRISPR-Cas proteins are isolated from a variety of natural contexts and therefore have different tolerances for elevated temperatures and optimal temperature ranges. These different tolerances for temperature are used to activate or inhibit the proteins at different stages to allow for other molecular processes, such as target amplification of nucleic acids from respiratory viruses (e.g., an influenza virus), to occur.

In DETECTR assays in which the target nucleic acid is from a respiratory virus (e.g., an influenza virus) and a Cas12 variant (SEQ ID NO: 37) programmable nuclease is used, the Type V protein Cas12 variant has a functional range between 25° C. and 45° C., with maximal activity at 35° C. For the Type V protein LbCas12a (SEQ ID NO: 27), the functional range is from 35° C. to 50° C. with peak activity around 40° C. For the Type VI protein LbuCas13a (SEQ ID NO: 131) the functional range is between 25° C. and 40° C. with maximal activity between 30° C. and 35° C. Type V proteins, such as the Cas12 variant and LbCas12a, are stable and functional at elevated temperatures. In DETECTR assays in which the target nucleic acid is from a respiratory virus (e.g., an influenza virus), the Cas12 variant exhibits activity at a temperature of 37° C. This temperature shifting is exploitable for use in isothermal amplification methods, where the amplification occurs at a higher temperature, but after lowering the reaction temperature the Cas protein is activated without compromising its functionality.

In DETECTR assays in which the target nucleic acid is from a respiratory virus (e.g., an influenza virus), the Cas12 variant is stable after exposure to elevated temperatures for 30 minutes and then lowering the reaction temperature to 37° C. The Cas12 variant is active in 0.5× NEBuffer4 (New England Biolabs)+0.05% Tween and 1× MBuffer3.

### Example 11

### Sample Preparation Protocol and Device Workflow

This example describes sample preparation protocol and device workflow. Collecting and processing material for diagnostic analysis is typically performed at a point of care facility or a clinical laboratory. There are minimal methods currently available for at home sample collection and nucleic acid extraction for diagnostic analysis. The devices disclosed herein provide an over the counter solution for nucleic acid extraction with or without nucleic acid amplification and with or without the DETECTR reaction. The resulting product from any or all of these modules is applied to a readout device for data collection and subsequent analysis.

A crude sample preparation protocol includes elution of a sample from a sample collection device (e.g. swab) into a buffer that will induce dissociation of the sample into its macromolecule components releasing the genomic nucleic acids. These components include any or all of the following: pH change, chaotropic salts and a detergent (Tween 20, Triton X-100, Deoxycholate, Sodium laurel sulfate or CHAPS. This protocol occurs in a stepwise work flow that would feed into a hand held device. In this device there is at least one chamber that contains the reagents components for the sample preparation protocol.

FIG. 7 shows individual parts of sample preparation devices of the present disclosure. Part A of the figure shows a single chamber sample extraction device: (a) the insert holds the sample collection device and regulates the step between sample extraction and dispensing the sample into another reaction or detection device, (b) the single chamber contains extraction buffer. Part B of the figure shows filling the dispensing chamber with material that further purifies the nucleic acid as it is dispensed is an option: (a) the insert holds the sample collection device and regulates the “stages” of sample extraction and nucleic acid amplification. Each set of notches (red, blue and green) are offset 90° from the preceding set, (b) the reaction module contains multiple chambers separated by substrates that allow for independent reactions to occur. (e.g., i. a nucleic acid separation chamber, ii. a nucleic acid amplification chamber and iii. A DETECTR reaction chamber or dispensing chamber). Each chamber has notches (black) that prevent the insert from progressing into the next chamber without a deliberate 90° turn. The first two chambers may be separated by material that removes inhibitors between the extraction and amplification reactions. Part C shows options for the reaction/dispensing chamber: (a) a single dispensing chamber may release only extracted sample or extraction/amplification or extraction/amplification/DETECTR reactions, (b) a duel dispensing chamber may release extraction/multiplex amplification products, and (c) a quadruple dispensing chamber would allow for multiplexing amplification and single DETECTR or four single amplification reactions.

FIG. 8 shows a sample work flow using a sample processing device. The sample collection device is attached to the insert portion of the sample processing device (A). The insert is placed into the device chamber and pressed until the first stop (green tabs meet black tabs) (B). This step allows the sample to come into contact with the nucleic acid extraction reagents. After the appropriate amount of time, the insert is turned 90° (C.) and depressed (D) to the next set of notches. These actions transfer the sample into the amplification chamber. The sample collection device is no longer in contact with the sample or amplification products. After the appropriate incubation, the insert is rotated 90° (E) and depressed (F) to the next set of notches. These actions release the sample into the DETECTR (green reaction). The insert is again turned 90° (G) and depressed (H) to dispense the reaction.

Examples of crude sample preparation protocols are summarized in TABLE 11.

TABLE 11

| | | | | | Incubation | Incubation |
|---|---|---|---|---|---|---|
| Name | Sample | HCl | Detergent | Urea | time | temperature |
| Low pH | Clinical | Yes | N/A | No | 15 minutes | RT |
| | reminant | | | | | |
| Low pH + heat | Clinical | Yes | N/A | No | 15 minutes | 60° C. |
| | reminant | | | | | |
| Deoxycholate | Clinical | No | Yes | No | 15 minutes | RT |
| | reminant | | | | | |
| Deoxycholate + | Clinical | No | Yes | No | 15 minutes | 60° C. |
| heat | reminant | | | | | |
| CHAPS | Clinical | No | Yes | No | 15 minutes | RT |
| | reminant | | | | | |
| CHAPS + heat | Clinical | No | Yes | No | 15 minutes | 60° C. |
| | reminant | | | | | |
| Deoxycholate + | Clinical | No | Yes | Yes | 15 minutes | RT |
| Urea | reminant | | | | | |
| Deoxycholate + | Clinical | No | Yes | Yes | 15 minutes | 60° C. |
| Urea + heat | reminant | | | | | |
| CHAPS + Urea | Clinical | No | Yes | Yes | 15 minutes | RT |
| | reminant | | | | | |
| CHAPS + Urea + | Clinical | No | Yes | Yes | 15 minutes | 60° C. |
| heat | reminant | | | | | |
| NucleoSpin | Clinical | No | Yes | Yes | 3 minutes | RT |
| Control | reminant | | | | | |

FIG. 9 shows extraction buffers used to extract Influenza A RNA from remnant clinical samples. Replicate remnant clinical samples were exposed to the reagents listed in TABLE 11 above. The extraction process was completed using the NucleoSpin Virus kit. qPCR analysis was performed to evaluate the quality and quantity of extracted RNA genome. The low pH condition resulted in RNA amounts equal to the sample extracted with the ‘gold standard’ kit (RT-pool).

FIG. 10 shows that low pH conditions allow for rapid extraction of Influenza A genomic RNA. Decreasing time of exposure to low pH conditions did not affect the efficiency of viral dissociation and subsequent extraction completed using the NucleoSpin Virus kit. The amount of extracted product was similar to the ‘gold standard’ extractions (RT-pool).

### Example 12

### Isothermal Amplification in CRISPR-Cas Diagnostics

This example describes methods of isothermal amplification in the CRISPR-Cas diagnostics of the present disclosure, including those diagnostics involving DETECTR assays. CRISPR diagnostics leverage the unique biochemical properties of Type V (e.g. Cas12) and Type VI (e.g. Cas13) CRISPR-Cas proteins to enable the specific detection of nucleic acids. These proteins are directed to their target nucleic acid by a CRISPR RNA (crRNA), which is also known as a guide RNA (gRNA). Once bound to a complementary target sequence, the Cas protein initiates indiscriminate cleavage of surrounding single-strand DNA or single-strand RNA. When coupled to a quenched fluorescence reporter or other cleavage reporter, fluorescent or other signal can be generated by the Cas protein only in the presence of the target nucleic acid. Alone these proteins are capable of detecting in the pM or fM range of target nucleic acid. When coupled to nucleic acid amplification set forth in this example and disclosed elsewhere herein, the sensitivity of CRISPR diagnostics was increased to the aM or zM range. PCR is a commonly used nucleic acid amplification method that generates double stranded DNA (dsDNA) when temperatures are cycled between two or three different temperatures. Nucleic acid amplification methods that function at single temperature are known as isothermal amplification. These methods include LAMP, RPA, SIBA, SDA, and NASBA. These methods can be coupled to reverse transcription (RT) which enables these methods to amplify RNA targets by first converting the RNA to cDNA through reverse transcription.

CRISPR based diagnostics using Type V (e.g., Cas12) and CasVI (e.g., Cas13) proteins were run using isothermal amplification methods of target nucleic acids to enable sensitive diagnostic assays.

RPA. Recombinase polymerase amplification (RPA) was used to amplify DNA sequences or RNA sequences by including a reverse transcription enzyme in the reaction (RT-RPA). RPA and RT-RPA can be used to generate an amplicon suitable for detection by Type V (e.g. Cas12) Cas proteins.

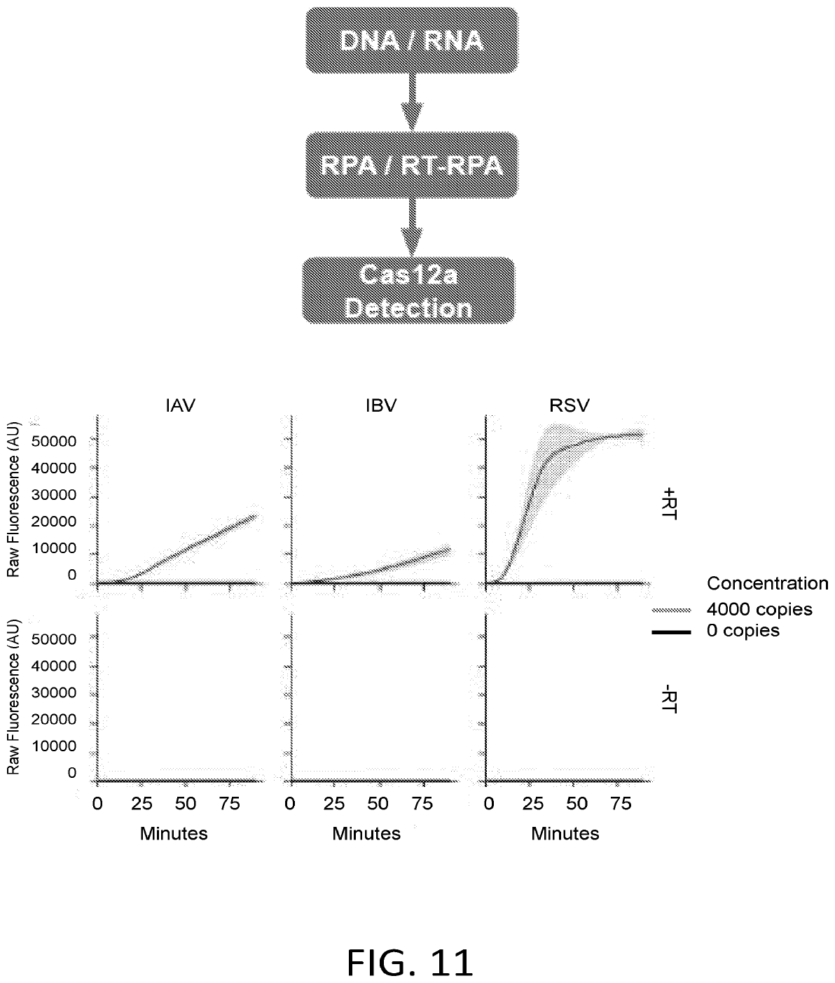

FIG. 11 shows the application of RT-RPA to the detection of Influenza A, Influenza B, and human Respiratory Syncytial Virus (RSV) viral RNA by Cas12a. By including a T7 promoter on one of the RPA primers, an in vitro transcription (IVT) reaction using an RNA polymerase to convert RNA to DNA step was performed after the RPA reaction to generate target RNA for detection by Type VI (e.g. Cas13) proteins. In FIG. 11, detection of RT-RPA amplicon was carried out from 4000 copies of Influenza A (IAV), Influenza B (IBV), and human respiratory syncytial virus (RSV) RNA using Cas12a. The RT-RPA reaction was performed at 40° C. for 30 minutes. Controls included no RT enzyme, no target control, and no primer control. Following the RT-RPA reaction, the RT-RPA amplicon was transferred to a Cas12a DETECTR assay.

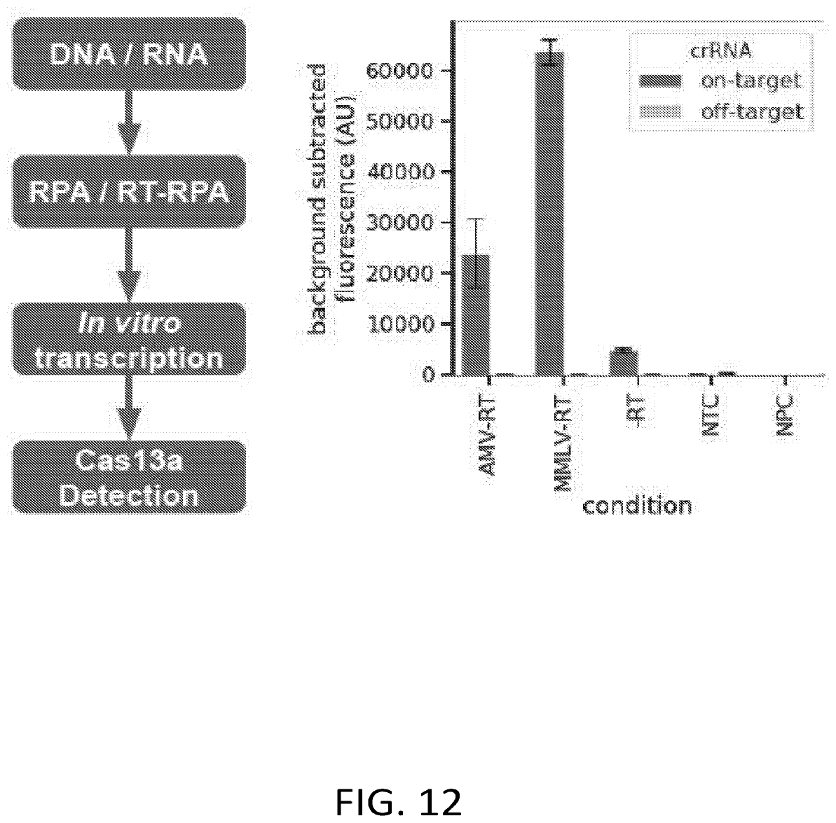

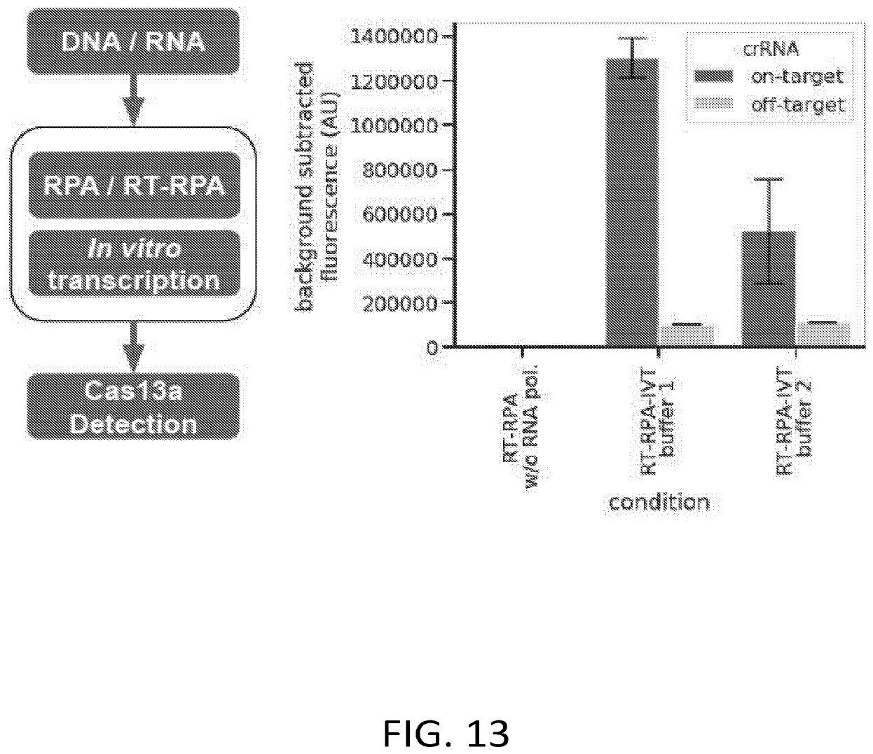

FIG. 12 shows the application of RT-RPA coupled with an IVT reaction enabling detection of viral RNA using Cas13a. In FIG. 12, detection of RT-RPA amplifcon was carried out from 2 fM of PPR virus RNA using Cas13a. The RT-RPA reaction was performed at 40° C. for 30 minutes. Several reverse transcriptase enzymes were evaluated for their compatibility with the RPA reaction. Controls included no RT enzymes, no target, and no primers. Following the RT-RPA reaction, the RT-RPA amplicon was transferred to an IVT reaction for generation of RNA at 37° C. for 10 minutes. The product of the IVT reaction was diluted and added to a Cas13a reaction at 37° C. On-target and off-target crRNAs were used to demonstrate specificity of the Cas13a reaction.

A “two-pot” DETECTR assay was carried out using RPA and Cas13a by combining the IVT reaction with the RT-RPA or RPA reaction to generate RNA simultaneously with the RPA reaction. FIG. 13 shows the production of RNA, as detected by Cas13a, from an RNA virus using an RT-RPA-IVT “two-pot” reaction. In the two-pot reaction, the first reaction was the RT-RPA-IVT, and the second reaction as the Cas13a detection assay. Components of the IVT (T7 RNA polymerase, NTPs) were added to a RT-RPA reaction in the presence of RNA transcription buffer (RPA rehydration buffer from Twist Dx “buffer 1”) or RPA rehydration buffer (20 mM imidazole, pH 7.5; 50 mM KCl; 5 mM MgCl2; BSA 10 μg/mL; 0.01% Igepal Ca-630; 5% glycerol “buffer 2”). As a control, RT-RPA without the RNA polymerase was added. 2 fM of PPR virus RNA was used as the target RNA in these reactions. The reaction proceeded for 15 minutes at 37° C. and on-target and off-target crRNAs were used to show specificity.

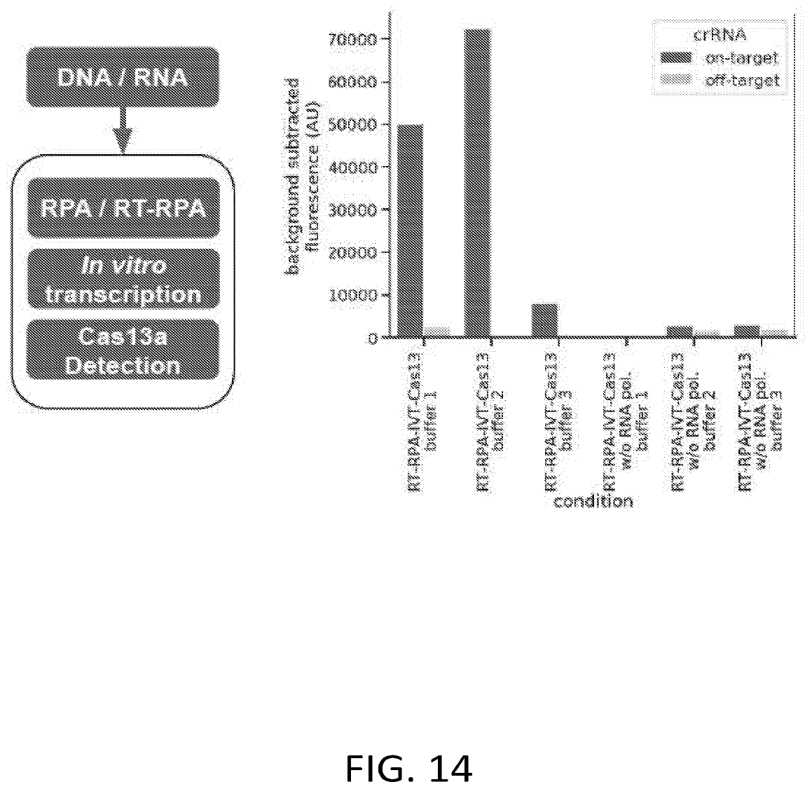

The IVT and Cas13a detection assay reactions were combined with RT-RPA or an RPA reaction to generate and detect RNA simultaneously in a “one-pot” assay. FIG. 14 shows the effect of various buffers on the performance of a one-pot Cas13a assay. Components for RT-RPA were combined in a single reaction with both components for IVT (T7 RNA polymerase, NTPs) and Cas13a detection (Cas13a enzyme, crRNA, fluorescent cleavage reporter). The reaction was run in three buffers (buffer 1: RPA rehydration buffer, buffer 2: Cas13a buffer, and buffer 3: Cas12a buffer (20 mM Tris-HCl, pH 8.0; 100 mM NaCl; 5 mM MgCl2; 1 mM DTT; 5% glycerol; 50 μg/mL heparin)). Reactions without the RNA polymerase were used as controls. In addition, specificity was shown by comparing a reaction with an on-target crRNA to a reaction with an off-target crRNA. The reaction was allowed to proceed at 40° C. for 10 minutes.

FIG. 15 shows the specific detection of viral RNA from the PPR virus that infects goats using the one-pot Cas13a assay. 500 aM of viral RNA was added to the reaction and the reaction was incubated at 40° C. As a control, an identical reaction without the T7 RNA polymerase (“PPRV-noIVT”; graph at right) was used to show the specific production of RNA for Cas13a to detect. An on-target and off-target crRNA was used to demonstrate assay specificity.

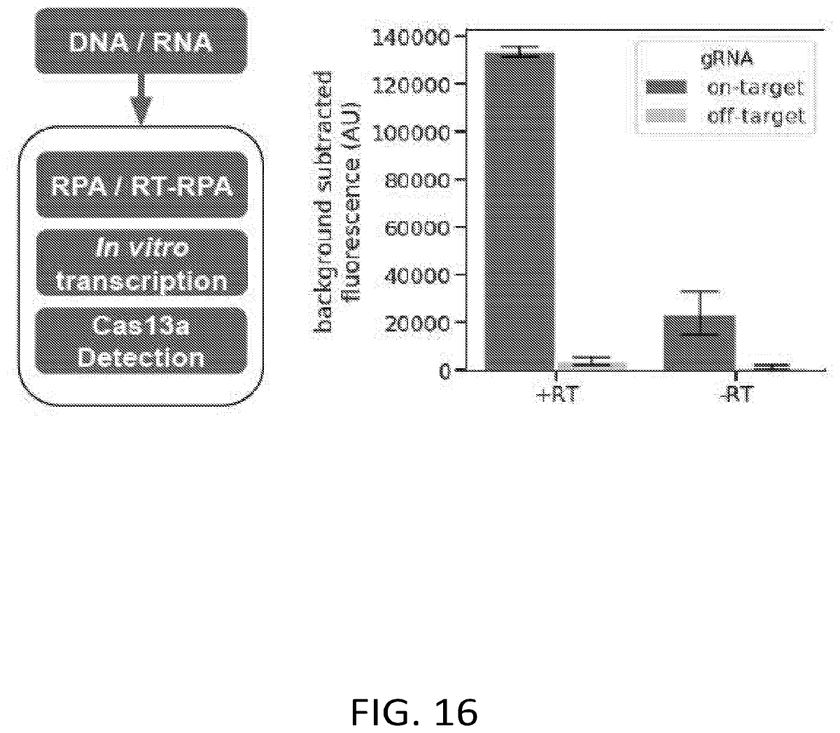

FIG. 16 shows the specific detection of Influenza B using the one-pot Cas13a assay run at 40° C. 40 fM of viral RNA was added to the reaction. As a control, an identical reaction without reverse transcriptase (labeled “−RT”) was used to show the specific production of RNA for Cas13a to detect. An on-target and off-target crRNA was used to demonstrate assay specificity.

FIG. 17 shows the tolerance of the one-pot Cas13a assay for the detection of RNA from the Influenza B virus in the presence and in the absence of a universal viral transport medium called universal transport media (UTM Copan) at 40° C. 40 fM of viral RNA was added to the reaction. As a control, an identical reaction without reverse transcriptase (−RT) was used to show the specific production of RNA for Cas13a to detect. An on-target and off-target crRNA was used to demonstrate assay specificity.

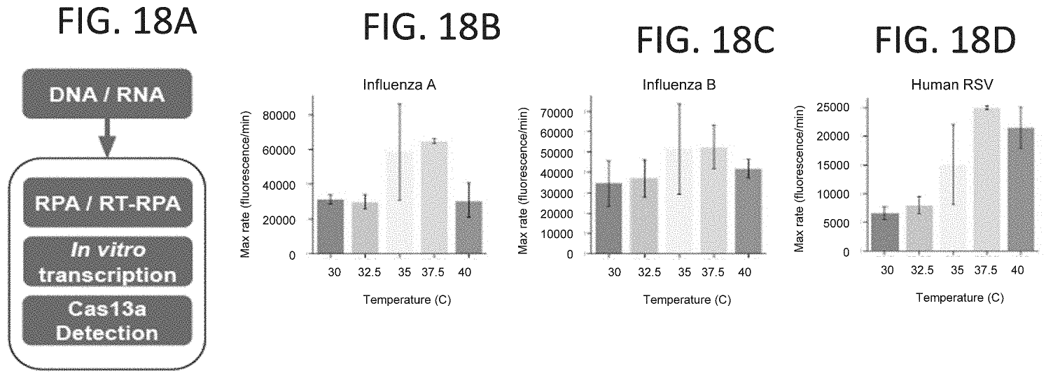

FIG. 18 shows the one-pot Cas13a detecting Influenza A (a), Influenza B (b), and human RSV (c) RNA at various temperatures. 100,000 viral genomes were added to the reaction and compared to reactions containing 0 copies. Reactions were run at either 30° C., 32.5, 35° C., 37.5° C., or 40° C. The assay was determined to be most robust between 35° C. and 37.5° C.

LAMP. Loop-mediated isothermal amplification (LAMP) was also used for amplifying a DNA sequences or RNA sequences in combination with a reverse transcriptase enzyme (RT-LAMP). LAMP reactions use a combination of four, five, or six primers to amplify the target DNA or cDNA from RNA. During the course of the LAMP reaction, concatemers of amplicons form. If RT-LAMP or LAMP amplicons contain sequence features that support Cas protein recognition (such as PAM or PFS), they can be used as target nucleic acids in CRISPR diagnostics.

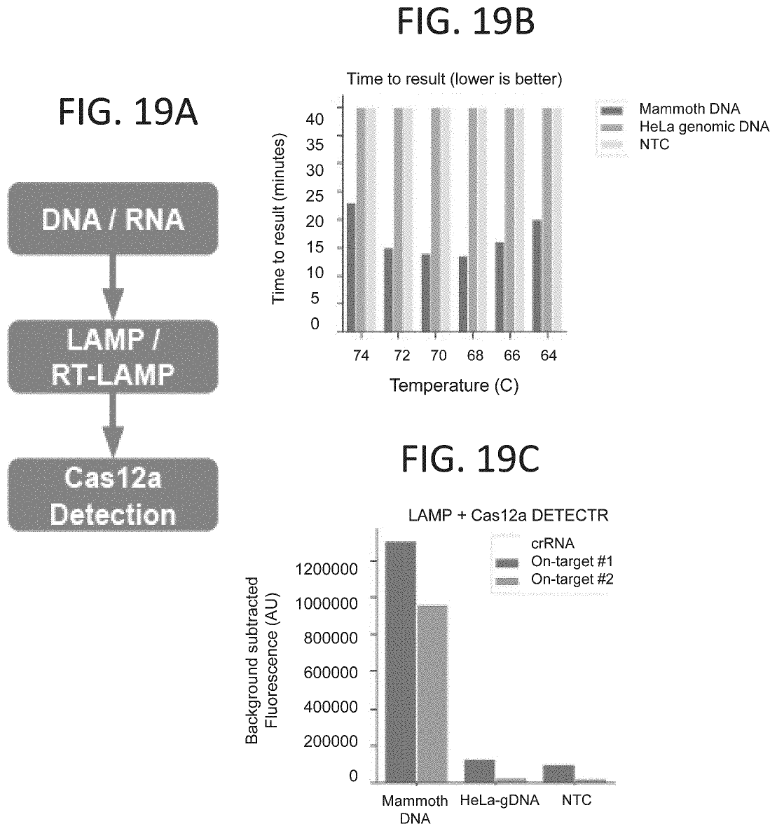

FIG. 19 shows the optimization of a LAMP reaction for the detection of an internal amplification control using a DNA sequence derived from the Mammuthus primigenius (Wooly Mammoth) mitochondria. In addition, FIG. 19 shows the specific detection of the LAMP amplicon by Cas12a using a variety of crRNAs. FIG. 19A shows a schematic of the workflow including providing DNA/RNA, LAMP/RT-LAMP, and Cas12a detection. FIG. 19B shows the time to result for LAMP reactions for an internal amplification control using a DNA sequence derived from the Mammuthus primigenius, as quantified by fluorescence. The time to result was determined by the time to reach half of max fluorescence for a reaction. Controls include off-target Hela genomic DNA and a no target control. FIG. 19C shows Cas12a specific detection at 37° C. of LAMP amplicon from the 68° C. temperature reaction. Two on-target crRNAs were tested. Specificity was shown by no detection from Hela genomic DNA amplicon or no template control (NTC) amplicon.

FIG. 20 shows the optimization of LAMP and Cas12 specific detection of the human POP7 gene (ACTCCGCAGCCCGTTCAGGACCCCGGCGCGGGCAGGGCGCCCACGAGCTGGCTGGC TGCTTGCACCCACATCCTTCTTTCTCTGGGACCTGGGGTCGCGGTTACTTGGGCTGGC CGGCGAACCCTTGAGTGGCCTGGCGGGGAGCGGGCCTCGCGCGCCTGGAGGGCCCT GTGGAACGAAGAGAGGCACACAGCATGGCAGAAAACCGAGAGCCCCGCGGTGCTG TGGAGGCTGAACTGGATCCAGTGGAATACACCCTTAGGAAAAGGCTTCCCAGCCGC CTGCCCCGGAGACCCAATGACATTTATGTCAACATGAAGACGGACTTTAAGGCCCA GCTGGCCCGCTGCCAGAAGCTGCTGGACGGAGGGGCCCGGGGTCAGAACGCGTGCT CTGAGATCTACATTCACGGCTTGGGCCTGGCCATCAACCGCGCCATCAACATCGCGC TGCAGCTGCAGGCGGGCAGCTTCGGGTCCTTGCAGGTGGCTGCCAATACCTCCACCG TGGAGCTTGTTGATGAGCTGGAGCCAGAGACCGACACACGGGAGCCACTGACTCGG ATCCGCAACAACTCAGCCATCCACATCCGAGTCTTCAGGGTCACACCCAAGTAATTG AAAAGACACTCCTCCACTTATCCCCTCCGTGATATGGCTCTTCGCATGCTGAGTACT GGACCTCGGACCAGAGCCATGTAAGAAAAGGCCTGTTCCCTGGAAGCCCAAAGGAC TCTGCATTGAGGGTGGGGGTAATTGTCTCTTGGTGGGCCCAGTTAGTGGGCCTTCCT GAGTGTGTGTATGCGGTCTGTAACTATTGCCATATAAATAAAAAATCCTGTTGCACT AGT, SEQ ID NO: 339) that is a component of RNase P. This sequence is present in human DNA and RNA and was used as a control for the efficiency of sample extraction (sample control). FIG. 20A shows a schematic of the workflow including providing DNA/RNA, LAMP/RT-LAMP, and Cas12a detection. FIG. 20B shows the time to result of a LAMP/RT-LAMP reaction for RNase P POP7 at different temperatures, as quantified by fluorescence. Time to result was determined by the time to reach half of max fluorescence for a reaction. Controls included off-target mouse-liver-RNA and a no target control. Hela total RNA and Hela genomic RNA were detected by the RT-LAMP and LAMP reactions, respectively. FIG. 20C shows three graphs demonstrating Cas12a specific detection at 37° C. of LAMP/RT-LAMP amplicon from the 68° C. temperature reaction. Two on-target crRNAs were tested and one off-target crRNA was tested. Specificity was shown by no detection of mouse total RNA amplicon or no template control (NTC) amplicon.

Cas12 was also used for the detection of RT-LAMP products. FIG. 21 shows the specific detection of three different RT-LAMP amplicons for Influenza A virus. The data from this experiment shows that the design of RT-LAMP primers around Cas12a compatible sites was important for the specificity of the experiment. The primer and crRNA were optimized and combined for specific detection of Influenza A (IAV) by RT-LAMP. Briefly, three different primer sets were tested for RT-LAMP that were specific to IAV. RT-LAMP reactions were performed either in the presence of IAV RNA or as a control with no template (NTC). For each amplicon, two on-target crRNAs and one off-target crRNA was used in a Cas12a detection assay at 37° C.

FIG. 22 shows the identification of optimal crRNAs for the specific detection of Influenza B (IBV) RT-LAMP amplicons. The RT-LAMP reaction was performed for 30 minutes at 60° C. in the presence of Influenza A (IAV) RNA, IBV RNA, or a no template control (NTC). For the resulting amplicons, three on-target crRNAs were used to determine which was most specific and efficient for the detection of Influenza B by Cas12a at 37° C.

The primers of an RT-LAMP or LAMP reaction were combined for multiplexed amplification. Because of the formation of concatemers during RT-LAMP and LAMP, it is difficult to differentiate between amplicons in a multiplex RT-LAMP or LAMP reaction by conventional means, as shown in FIG. 23. Multiplexed RT-LAMP for Influenza A (IAV) and Influenza B (IBV) was carried out for 30 minutes at 60° C. RT-LAMP reactions were incubated with 10,000 viral genome copies of IAV, IBV, or both IAV and IBV. A no-target control (NTC) was used that contained 0 viral genome copies. 0.5 uL of the RT-LAMP product after the 30 minute incubation was run on a 1% agarose gel. FIG. 23 shows the results of the 1% agarose gel with bands showing the products of the RT-LAMP reaction. As seen in the gel, it is difficult to differentiate between the IAV, IBV, and IAV +IBV samples. The application of Type V (e.g. Cas12) enzymes identified which amplicons were amplified in a multiplexed RT-LAMP or LAMP reaction. FIG. 24 shows Cas12a discrimination between amplicons from a multiplex RT-LAMP reaction for Influenza A and Influenza B. FIG. 24A shows a schematic of the workflow including providing viral RNA, multiplexed RT-LAMP, and Cas12a influenza A detection or Cas12a influenza B detection. FIG. 24B shows Cas12a detection of RT-LAMP amplicons after 30 minute multiplexed RT-LAMP amplification at 60° C. Multiplexed amplification contained primer sets for Influenza A (IAV) and Influenza B (IBV). Reactions contained 10000 viral genome copies or 0 copies as a control. Targets for IAV only, IBV only, and IAV and IBV combined were used. FIG. 24C shows background subtracted fluorescence at 30 minutes of Cas12a detection at 37° C. of RT-LAMP amplicons for 10,000 viral genome copies of IAV and IBV. crRNAs specific for IAV and IBV enable discrimination for which viral sample was present. Similarly, FIG. 25 shows Cas12a discrimination between a triple multiplexed RT-LAMP reaction for Influenza A, Influenza B, and the Mammuthus primigenius (Wooly Mammoth) mitochondria internal amplification control sequence after 30 minutes of multiplexed RT-LAMP amplification at 60° C. Multiplexed amplification contained primer sets for Influenza A (IAV), Influenza B (IBV), and the Mammoth internal amplification control (Mammoth IAC). Reactions contained 100,000 viral genome copies or 500 aM of the IAC. Targets for IAV only, IBV only, multiplexed IAV+IBV, and multiplexed IAV+IBV+Mammoth IAC were used. Cas12a detection assays at 37° C. with IAV, IBV, and Mammoth IAC specific crRNAs were performed to differentiate the amplicons from the multiplexed reactions.

By including a T7 promoter sequence in the forward inner primers (FIP) or backward inner primers (BIP) of a LAMP or RT-LAMP reaction, the resulting amplicon can be added to an in vitro transcription reaction to generate RNA, as shown in the schematic in FIG. 26. This RNA can be used in a Type VI (e.g. Cas13) detection assay. FIG. 26A shows a schematic illustrating the identity of the primers used in LAMP and RT-LAMP. Primers LF and LB are option in some LAMP and RT-LAMP designs, but generally increase the efficiency of the reaction. FIG. 26B shows a schematic illustrating the position and orientation of the T7 promoter in a variety of LAMP primers.

FIG. 27 shows that a T7 promoter can be included on the F3 or B3 primers (outer primers), or FIP or BIP primers for Influenza A. However, only T7 promoters located in the FIP or BIP primers are capable of generating enough RNA to enable a Cas13a detection assay. FIG. 27A shows a schematic of the workflow including providing DNA/RNA, LAMP/RT-LAMP, in vitro transcription, and Cas13a detection. FIG. 27B shows the time to result for RT-LAMP reactions for Influenza A using different primer sets, as quantified by fluorescence. Each primer set contained a T7 promoter sequence in a different position. Time to result was determined by the time to reach half of max fluorescence for a reaction. No target was used as a specificity control. The results demonstrated that the B3+T7 and BIP+T7 sense primer sets worked best for RT-LAMP reaction. The reaction was performed at 68° C. for 30 minutes. FIG. 27C shows in vitro transcription (IVT) with T7 RNA polymerase of the product of the RT-LAMP reactions for Influenza A using different primer sets at 37° C. for 10 minutes. A Cas13a detection assay at 37° C. was then used to detect the RNA products from the IVT reaction. Three different on-target crRNAs were used along with an off-target crRNA to demonstrate specificity. The BIP+T7 sense and antisense primer sets worked best for RNA production, along with on-target crRNA #2. Thus, the BIP+T7 sense primer set in conjunction with crRNA #2 worked best for the detection of RNA after a RT-LAMP reaction followed by an IVT reaction.

SIBA. Strand invasion based amplification (SIBA) is another isothermal method that can be used. FIG. 28 shows the detection of a RT-SIBA amplicon for Influenza A by Cas12. In SIBA and RT-SIBA reactions for Cas12, the guide RNA is not complementary to the invasion oligo and the amplicon contains a PAM. The RT-SIBA reaction was performed at 41° C. for 60 minutes with a starting RNA concentration of 500 aM. Controls for the RT-SIBA reaction included a no target control and a no primer control. After the completion of the RT-SIBA reaction, 2 μL of amplicon was added to a 20 μL Cas12a detection reaction. On-target and off-target crRNAs were used to show specific detection of by Cas12.

### Example 13

### Optimization of Assay Conditions for CRISPR DETECTR-based Diagnostic Assays for Detection of Target Nucleic Acids from Respiratory Viruses

This example describes optimization of assay conditions for the CRISPR-Cas DETECTR-based diagnostic assays disclosed herein for the detection of target nucleic acids from respiratory viruses (e.g., an influenza virus). The components of the DETECTR reaction, such as protein concentration, crRNA, and buffer components impact the rate and efficiency of the reaction. Optimization of the buffers allows for the development of an assay with increased sensitivity and specificity.

Cas13M26 (LbuCas13a (SEQ ID NO: 131)) performs optimally in DETECTR reactions in buffers with decreased amounts of tRNA without changing the stability of the reaction. Decreasing the amount of tRNA in the reaction or eliminating it completely, increases the efficiency of the Cas13a detection assay without dramatically changing the stability of the reaction in the absence of activator. Buffers in which Cas13a exhibits activity in a DETECTR assay for detection of nucleic acids from a respiratory virus (e.g., influenza virus) lack or have low amounts of urea and SDS. Additionally, Cas13a exhibits activity in DETECTR assays for detection of nucleic acids from a respiratory virus (e.g., influenza virus) in buffers comprising NaCl or KCl, with 30 mM salt or below, and/or with 0-10 mM DTT in buffers containing either NaCl or KCl. Cas13a also exhibits activity, as measured by fluorescence, for a number of reporters, including a “U5” reporter (/5-6FAM/rUrUrUrUrU/3IABkFQ/ (SEQ ID NO: 1)), a “UU” reporter (/56-FAM/TArUrUGC/3IABkFQ/ (SEQ ID NO: 381)), and a reporter with the same nucleotide sequence as the “U5” reporter but with a different fluorophore and quencher, “TYE665U5” (/5-TYE665/rUrUrUrUrU/3IABkRQ/ (SEQ ID NO: 1)). Optimal buffer compositions and pH for Cas13a DETECTR assays include buffers with a pH around 7.5 and buffers imidazole, phosphate, tricine, and SPG.

Cas13a performance is improved in NEBuffer2 (NEBuffer 2.1; 1× Buffer Components, 50 mM NaCl, 10 mM Tris-HCl, 10 mM MgCl2, 100 μg/ml BSA, pH 7.9@25° C.) and Cutsmart (1× Buffer Components, 50 mM Potassium Acetate, 20 mM Tris-acetate, 10 mM Magnesium Acetate, 100 μg/ml BSA, pH 7.9@25° C.). Cas13a performs optimally in MBufferl. 1× MBuffer1 includes 20 mM imidazole pH 7.5, 50 mM KCl, 5 mM MgCl2, 10 μg/μL BSA, 0.01% Igepal Ca-630, and 5% glycerol. Additionally, Cas13a performance is improved in buffers comprising 5% glycerol, BSA, and NP-40 improve Cas13a DETECTR assay. NP-40 (Igecal-Ca 630) increases the efficiency of the Cas13a detection assay and small amounts of BSA also improve the performance of the assay. Concentrations of 0.05% to 0.0625% NP-40 are the most optimal and concentrations of 2.5 to 0.625 μg/mL BSA are desirable.

Buffers lack compounds that inhibit the performance of the Cas13a DETECTR assay, including: beryllium sulfate, manganese chloride, zinc chloride, tri-sodium citrate, copper chloride, yttrium chloride, 1-6-Diaminohexane, 1-8-diaminooctane, ammonium fluoride, ethanolamine, lithium salicylate, magnesium sulfate, potassium cyanate, and sodium fluoride.

LbCas12a (SEQ ID NO: 27) exhibits optimal activity in DETECTR assays for detection of target nucleic acids from respiratory viruses (e.g., influenza viruses) using buffers with a pH 8.0 and the following buffer types: AMPD, BIS-TRISpropane, DIPSO, HEPES, MOPS, TAPS, TRIS, and tricine buffers. LbCas12a exhibits activity in DETECTR assays with low KCl concentrations (0-40 mM or less than 20 mM salt and less KCl).

A Cas12 variant (SEQ ID NO: 37) performs optimally in a pH of 7.5 and in buffers including DIPSO, HEPES, MOPS, TAPS, imidazole, and tricine. The Cas12 variant performs best at a salt concentration of around 4 mM (ranging from 2-10 nM) and exhibits increased activity in buffers with MgOAc and KOAc (acetate buffers), in comparison to buffers with MgCl and KCl. Additionally, the Cas12 variant is inhibited by heparin and prefers low salt.

Buffers lack specific compounds inhibiting the performance of the Cas12 variant DETECR assay include: benzamidine hydrochloride, beryllium sulfate, manganese chloride, potassium bromide, sodium iodine, zinc chloride, di-ammonium hydrogen phosphate, tri-lithium citrate, tri-sodium citrate, cadmium chloride, copper chloride, yttrium chloride, 1-6 diaminohexane, 1-8-diaminooctane, ammonium fluoride, and ammonium sulfate. Compounds that increase assay performance included: polyvinyl alcohol type II, DTT, DMSO, polyvinylpyrrolidone K15, polyethylene glycol (PEG) 600, and polypropylene glycol 400.

SNP differentiation is stronger for the Cas12 variant along the 3′ end of a crRNA (distal from the PAM). LbCas12a (SEQ ID NO: 27) displays strong mutation sensitivity at all positions along target sequences, and sensitivity on the PAM proximal (complementary to the 5′ end of the crRNA target sequence) end and is more sensitive to mutations in this region and mutation sensitivity is target site dependent.

### Example 14

### Lateral Flow Test Strips for Visual Detection of Target Nucleic Acids from a Respiratory Virus in DETECTR Reactions Using CRISPR-Cas Systems

This example describes a lateral flow test-strip for visual detection of target nucleic acids from a respiratory virus (e.g., an influenza virus) in DETECTR reactions using CRISPR-Cas systems. Visual readouts for the DETECTR reaction are developed to have a low-cost format and be amenable to high-volume manufacturing. Described here are custom-made lateral flow strips. Colloidal gold nanoparticles are conjugated to antibodies and the gold nanoparticles serve as a visual readout in the assay. Two commercially available lateral flow strips are tested including: (1) Millenia Hybridetect 1, TwistDx (UK, now part of Abbott) and (2) PCRD, Abingdon Health (UK).

Results are collected by: (1) visual inspection of the strips, and (2) obtaining a cell-phone-camera picture of the strips. Unlike commercially available lateral flow test strips, the custom-made lateral flow strip design disclosed herein includes a new type of CRISPR-Cas reporter molecule, which is made of (1) a 6-Fluorescein (FAM) moiety; (2) a biotin moiety; and (3) a DNA-based oligo linker, which are irreversibly conjugated to the DETECTR reaction chamber upstream of the reaction.

Lateral Flow Strips for Read Out of a Cas12 variant and LbCas12a. Lateral flow strips are tested for readout of a Cas12 variant (SEQ ID NO: 37) and LbCas12a (SEQ ID NO: 27). Complexing reactions include final concentrations of 40 nM of crRNA per reaction, 40 nM of final protein per reaction, and 500 nM of reporter per reaction. Complexing reactions are incubated at 37 C for 30 min, the reporter substrate is added, and 15 uL of the complexing reactions are aliquoted into PCR tubes. 5 uL of diluted PPR virus PCR product is added and the target (e.g., a sample containing a target nucleic acid from a respiratory virus) and complex are incubated at 37 C for 20 min. 100 uL of Mllenia GenLine Dipstick Assay Buffer (Tween or Triton) is added and the dipstick is inserted into the solution with target and complex. Test strips are photographed and the top band was quantified using ImageJ.

MNT-Lateral Flow, Au NP Conjugation. Anti-FAM and anti-ROX polyclonal antibodies are conjugated to gold nanoparticles for downstream use in the custom made lateral flow strips. Materials include Corning Spin-X UF 500 uL Concentrators and a Gold in a Box Conjugation kit. A 0.5× buffer solution is prepared by diluting PBS, pH 7.2 (1×) in 1:1 with nuclease-free water. 100 ul of the MNT antibody and 100 ul of a FITC antibody are used. Spin concentrators are used to exchange native buffer from 0.1M Tris glycerine, pH 7 with 10% glycerol to 0.5× PBS for both antibodies. Washes with 100 ul of 0.5× PBS are carried out and the concentrators were spun for 1.5 min at 18,000 rcg (×g) for each wash. Antibodies are eluted in 100 ul of 0.5× PBS. Gold conjugation is carried out as per manufacturer's instructions. Tubes are labeled MNT1-10 and FITC1-10 and 7 ul of each antibody was added. Reactions are incubated for 30 min in a shaking incubator at room temperature. The reaction is stopped by adding 50 uL of a BSA blocking buffer to each tubes, and tubes are stored at 4 C.

Lateral Flow Strips for Read Out of LbuCas13a. Lateral flow strips are tested for readout of LbuCas13a. TwistDx lateral flow strips are used to test the FAM-US-Biotin (SEQ ID NO: 1) (rep71 reporter). Assays are run at room temperature at a variety of target concentrations. Complexing reactions include final concentrations of 40 nM of crRNA per reaction, 40 nM of final protein per reaction, and 500 nM of reporter per reaction. Complexing reactions are incubated at 37 C for 30 min. Dilutions of the target are added to the reaction including at 10 nM, 1 nM, 0.1 nM, 0.01 nM, and no target. 30 uL of the complexing reaction is added to the target and incubated for 15 minutes at room temperature. The reaction is placed on ice and 10 uL of the reaction is pipetted directly onto the lateral flow sample area. 50 uL of Milenia GenLine Dipstick Assay buffer is added and the strip was photographed.

Conjugation of 3′Amino Reporter to NHS Beads Using Kit. An NHS FlexiBind Magnetic Bead Kit is used to conjugate the 3′amino modified lateral flow reporter allowing for the intended usage of the lateral flow devices (Milenia Hybrid), where the ligand is detected first and the control line serves as the flow control. The sequence of the reporter used is /56-FAM/*/iBiodT/*AATTAATTAATTAATTAATT/3AmMO/ (SEQ ID NO: 372).

Bead conjugation is carried out as follows. Rep75 is resuspended to 100 μM in Wash/Coupling Buffer (PBS, pH 7.4). 32.5 nmol is delivered from IDT and 5.4 nmol (54 μL) of rep75 is used. 20% NHS FlexiBind Magnetic beads are resuspended by vortexing for 20 seconds. 100 μL of bead slurry is pipetted into a 1.5 mL microcentrifuge tube. Magnetic beads are pelleted on the magnetic stand until the solution became clear. Storage buffer is removed and discarded. 100 μL of ice-cold Equilibration buffer (1 mM HCl) is immediately added. The reaction is removed from the magnet and vortexed for 20 seconds, then placed back on the magnet to pellet beads. The supernatant is removed and discarded and 54 μL of 100 μM rep75 in PBS is added. Beads are incubated at room temperature with interval mixing: 2 min rest, 15 sec mix at 1200 rpm for 2 hours. Tubes are placed in a magnetic stand to allow the beads to migrate to the magnet. Unbound ligand is removed and saved for analysis.

0.5 μL raw reporter is measured in 20 μL NFW vs. 0.5 μL post-conjugation supernatant in 20 μL NFW on a plate reader until it is no longer visibly green. 500 μL of Quench buffer is added, vortexed for 30 seconds, and pelleted with a magnetic rack. The supernatant is discarded and the sample is washed 5 times. Beads are resuspended in 500 μL of Quench Buffer and incubated for 1 hour at room temperature. The beads are pelleted with a magnetic rack and the buffer is removed and discarded. The beads are resuspended in 100 μL of Wash/Coupling Buffer (PBS, pH 7.4) and the beads are kept on ice in dark tube.

Testing uncleaved/unconjugated reporter with lateral flow is carried out using 2× NG-40-B009 Naked Gold Sol beads—40 nm—15 OD—9 mL, FITC antibody (Invitrogen TB265150), anti-IgG (Invitrogen A16098), Streptavadin (NEB N7021S), pH 8.8, and three batches—Batch 1: AU (5 μL)→anti-IgG (μL)→Strep (0.5 μL), Batch 2: AU (5 μL)→strep (1 μL)→anti-IgG (1 μL), and Batch 3: AU (2.5 μL)→strep (1 μL)→anti-IgG (1 μL).

Test beads with a Cas12 variant (SEQ ID NO: 37) by first complexing reaction. Reactions are run with final concentrations of 40 nM crRNA per reaction, 40 nM protein per reaction, and 100, 250, 500, or 1000 nM reporter per reaction. The complex is incubated at 37 C for 30 min. The 40 μM stock of beads is diluted to 1:10 to 4 μM. Reporter beads are added to 5 μL PPRV diluted PCR product or NFW, 15 μL of complexing reaction was added to target. The reaction is incubated at 37 C for 30 min with shaking at 2000 rpm in Thermomixer. Beads are pelleted with magnetic rack for 2 minutes. 10 μL of reaction is transferred to a new tube, 50 μL of Dipstick Assay Buffer is added, and 60 μL diluted reaction is placed on magnet before adding solution to lateral flow strips. Reactions are run on Milenia flow strips.

FIG. 29 shows the layout of a Milenia commercial strip with a typical reporter. This schematic shows an analyte-independent universal dipstick with a sample application region at right followed by a wicking region immediately to the left, followed to the left by a region containing a biotin ligand, followed to the left by a region spotted with anti-rabbit antibody. The sample and analyte-specific solution are incubated with analyte detectors bearing a biotin or FITC. Samples are run on the strip. A positive result shows two bands—the left most band is from the control band and is due to binding of anti-FITC antibody coated gold nanoparticles to an anti-rabbit antibody. The right band is from the test band itself and is due to binding by the biotin ligand to an analyte detector bearing biotin, where the detector complexes the analyte and wherein the analyte is further complexed to another detector bearing FITC, which is then bound to the anti-FITC antibody coated gold particle. In the negative result—only one band is seen at the control line.

FIG. 30 shows the layout of a Milenia HybridDetect 1 strip with an amplicon. This schematic shows at top PCR amplicon using FAM and biotin primers at the right end of the top figure. In the case of a positive result, the strip shows two bands—this PCR amplicon binds to a moiety immobilized at the test line, and the FAM molecule (shown as a start) binds to an anti-FAM antibody coated particle. To the left of the test line is a flow control line, containing anti-rabbit antibody which binds to anti-FAM antibody coated nanoparticles. In the case of a negative result, the strip shows one band—that is, just binding of anti-FAM antibody coated nanoparticles bound to anti-rabbit antibody immobilized on the test strip.

FIG. 31 shows the layout of a Milenia HybridDetect 1 strip with a standard Cas reporter. A positive result is shown at top where a Cas protein cleaves the standard reporter, and only one band is seen—due to binding of the anti-FAM antibody coated nanoparticles to anti-rabbit antibody spotted on the strip. A negative result is shown at bottom where the intact reporter binds to a moiety immobilized on the strip and all anti-FAM antibody coated nanoparticles bind at the control line to the FAM molecule on the intact Cas reporter. Results of running samples with target nucleic acids and with a water only control show that even with the water only control, a false positive band appears at the test line.

FIG. 32 shows a modified Cas reporter comprising a DNA linker to biotin-dT (shown as a pink hexagon) bound to a FAM molecule (shown as a green start). This entire modified Cas reporter was conjugated to magnetic beads or the surface of the reaction chamber, which was upstream of the strip. This is shown in the schematic as immobilization of the modified Cas reporter to the substrate of the DETECTR chamber/bead. During cleavage by a Cas (shown as a yellow pac-man), the biotin-FAM molecule is released from the DNA linker. Unlike other assay formats, this particular assay format contains the entire Cas cleavage reaction to the reaction chamber. In this assay format, the test-line is the actual test line and the control line is a true control line. FIG. 33 shows the layout of Milenia HybridDetect strips with the modified Cas reporter. At top, a positive result is shown, where in the Cas reaction chamber, the Cas protein cleaves the DNA linker segment of the modified Cas reporter. The biotin-dT/FAM molecule is released and flows down the test strip binding to streptavidin coated on the test line. An anti-FAM antibody coated gold nanoparticle binds to the biotin-DT/FAM reporter at the test line. Additionally the anti-FAM antibody coated gold nanoparticle binds to anti-rabbit antibody coated at the flow control line. At bottom, a negative result is shown where only the anti-FAM antibody coated gold nanoparticle binds to anti-rabbit antibody coated at the flow control line.

FIG. 34 shows an example of a single target assay format (to left) and a multiplexed assay format (to right). At the top are diagrams showing a schematic of the assay prior to use, anti-FAM antibody coated gold nanoparticles only (on left) or anti-FAM antibody coated gold nanoparticles and anti-ROX antibody coated gold nanoparticles (to right) are upstream of the control and test lines. The control lines are spotted with streptavidin and the test lines are spotted with only target A (left) or target A and target B (right). Assays with positive results are shown in the middle schematic and assays with negative results are shown in the lowest schematic.

FIG. 35 shows another variation of an assay prior to use (top), an assay with a positive result (middle left), an assay with a negative result (middle right), and a failed test (bottom). In this assay the flow control is at the left most end of the strip, followed by the test line coated with anti-IgG rabbit antibody, followed by the control line coated with streptavidin, followed by gold nanoparticles coated with anti-FAM or anti-biotin antibodies. The Cas reporters are upstream of the strip in a reaction chamber. If cleaved the Cas reporter is cleaved (positive result), FAM molecules bind to the anti-FAM coated gold nanoparticles, which subsequently bind at the test line and anti-biotin antibody coated nanoparticles bind at the control line to the DNA/RNA linker/biotin construct. If the Cas reporter is not cleaved (negative result), the intact reporter binds to streptavidin at the control line, where they are subsequently bound by anti-FAM coated gold nanoparticles.

Gold nanoparticle conjugation to anti-biotin antibody. A 100 ul aliquot of anti-biotin antibody is used, with the antibody suspended in nuclease free water. 7 ul of the dilute antibody in solution is added to tubes and reactions were incubated for 30 min in a shaking incubatory at room temperature. The reaction in each tube is stopped with the addition of 50 ul of the BSA blocked buffer and the tubes were stored at 4 C.

### Example 15

### Conjugation of Oligonucleotides to Peptides/Enzymes for Downstream Use in an Invertase-Coupled Assay for Amperometric detection of Target Nucleic Acids from a Respiratory

Virus in DETECTR Reactions Using CRISPR-Cas Systems

This example describes a conjugation method for oligonucleotides to peptides/enzymes for downstream use in an invertase coupled assay for amperometric detection of target nucleic acids from a respiratory virus (e.g., influenza virus) in DETECTR reactions using CRISPR-Cas systems. The methods disclosed herein are developed as alternatives to fluorescence and lateral-flow-immunochromatography readouts of DETECTR reactions and include efficient conjugation of an invertase enzyme to a DETECTR reporter using a 3′ thiol modification. The CRISPR-Cas reporter molecule for use in the invertase-coupled assay for amperometric detection of DETECTR reactions includes (1) a 5′-Biotin moiety and (2) a 3′-invertase enzyme. The sequence of the oligo was /5Biosg/TTTTTTTTTTTTTTTTTTTT/3ThioMC3-D/ (SEQ ID NO: 373) and the invertase enzyme is conjugated at the 3′ end. Reagents for the conjugation include invertase from Baker's yeast (S. Cerevisiae), streptavidin magnetic beads, Tris(2-carboxyethyl)phosphine hydrochloride (TCEP), N-Succinimidyl 4-(maleimidomethyl)cyclohexanecarboxylate, SMCC (SMCC), 1M sodium phosphate buffer, pH 7.2, 2-(NMorpholino)ethanesulfonic acid (MES), sodium chloride 5M sterile, and biotin labelled oligos.

Buffer and solution preparation. Buffers include (1) 0.1M Phosphate Buffer, no NaCl, pH 7.2, (2) 0.1M Phosphate Buffer, 0.1M NaCl, pH 7.2, (3) 0.05M MES Buffer, pH 5.5, and (4) 0.05M MES Buffer, with 0.1M NaCl, pH 5.5. TCEP solution, SMCC solution, and invertase solution is prepared from solids. The DNS reagent is also prepared.

Thiol activation of DNA oligo. The thiol-biotin-labelled oligos (15 μL, 1 mM in water) are mixed with TCEP (3 μL, 0.5M in water) in a 1.5-mL microcentrifuge tube. The reaction volume is made up to 30 μL with the addition of 12 μL of eqivalent buffer. The following twelve reactions are prepared: MB406 with low pH, no salt buffer; MB406 with low pH and salt buffer; MB406 with PBS, no salt; MB406 with PBS and salt buffer; MB407 with low pH, no salt buffer; MB407 with low pH and salt buffer; MB407 with PBS, no salt; MB407 with PBS and salt buffer; MB408 with low pH, no salt buffer; MB408 with low pH and salt buffer; MB408 with PBS, no salt; and MB408 with PBS and salt. In each buffer, the volume of DNA oligos is 15 μL, the volume of TCEP was 3 μL, and the volume of buffer was 12 μL. The reaction is incubated for 3-5 hours in the shaking incubator at 37° C. The reaction is stopped by snap freezing in liquid nitrogen. Microcentrifuge tubes are stored at −20° C. until the next step of the reaction. Thiol-activated oligo tubes are removed from the freezer 3 hours prior to conjugation to activated invertase/or other activated proteins/peptides. The tube is first incubated at 37° C. for 3 hours and then used in the conjugation reaction

SMCC activation of invertase enzyme. A fresh solution of invertase lab stock bottle is prepared. 10 mg of solid was weighed in a clean 1.5 mL microcentrifuge tube, 860 μL of buffer A (0.1M NaCl, 0.1M sodium phosphate buffer, pH 7.2) is added to make a solution of 20 mg/mL. 1 mg of SMCC is added to a 1.5 mL microcentrifuge tube and the reaction is initiated by addition of invertase solution (400 μL, 20 mg/mL in 0.1M NaCl, 0.1M sodium phosphate buffer, pH 7.2). The reaction is incubated in the shaking incubator at 37° C. for 24 hours.

Cleanup of SMCC-activated invertase. The reaction is removed from the shaking incubator (37° C.) after 23 hours and 15 minutes. SMCC-activated invertase is washed 8× and resuspended in 400 μL of buffer. Protein is quantified by the BCA method.

Re-activation of thiol-DNA oligo. The oligo is removed from −20° C. and incubated in the shaking incubator at 37° C. The reaction is initiated and incubated in the shaking incubator (37° C.) for 48 hours. The reaction is removed from the incubator and each reaction contained (1) 35 μL of invertase solution, and (2) 30 μL of thiol-DNA oligo solution.

Binding with streptavidin beads. 12.5 μL of streptavidin beads is mixed with 50 μL of the biotinylated DNA oligo previously conjugated with invertase enzyme in a 1.5-mL microcentrifuge tube. The reaction is incubated for 5 minutes at room temperature, beads were washed 5× with 50-μL aliquots of Buffer A on a magnetic rack to remove any unbound DNA oligo from the solution, and eluent from all the washes was checked for invertase activity (and thus inefficient binding between streptavidin and biotin molecules). During the last wash, beads are resuspended with 50 μL of Buffer A and beads were stored at 4° C.

Incubation with DNS/Sucrose. A reaction is prepared containing 5 μL of 20% sucrose, 30 μL DNS reagent, 25 μL of biotynlated DNA with invertase moiety. A color change is observed after incubation at high heat (95 C).

DNA-Invertase Conjugation. Conjugation is carried out using a heterobifunctional linker sulfo-SMCC. To 30 uL of 1 mM thiol-DNA in Millipore water, 2 uL of 1 M sodium phosphate buffer at pH 5.5 and 2 uL of 30 mM TCEP in Millipore water are added and mixed. This mixture is kept at room temperature for 1 hour and then purified by Amicon-10K using Buffer A (0.1 M NaCl, 0.1 M sodium phosphate buffer, pH 7.3, 0.05% Tween-20) without Tween-20 by 8 times. For invertase conjugation, 400 uL of 20 mg/mL invertase in Buffer A without Tween-20 is mixed with 1 mg of sulfo-SMCC. After vortexing for 5 minutes, the solution is placed on a shaker for 1 hour at room temperature. The mixture is then centrifuged and the insoluble excess sulfo-SMCC was removed. The clear solution is then purified by Amicon-100K using Buffer A without Tween-20 by 8 times. The purified solution of sulfo-SMCC-activated invertase is mixed with the above solution of thiol-DNA. The resulting solution is kept at room temperature for 48 hours. To remove un-reacted thiol-DNA, the solution is purified by Amicon-100K 8 times using Buffer A without Tween-20. Conjugation is also carried out using homobifunctional linker PDITC. To 60 uL of 1 mM amine-DNA in Millipore water, 30 uL of Buffer B (0.1 M sodium borate buffer, pH 9.2) are added and mixed. This solution is further mixed with 20 mg of PDITC dissolved in 1 mL DMF. The resulting solution is placed on a shaker and kept at room temperature in the dark for 2 hours. After that, the solution is mixed with 6 mL of Millipore water and 6 mL 1-butanol. After centrifuging for 15 min, the upper organic phase is discarded. The aqueous phase is then extracted with 4 mL 1-butanol three times, and purified by Amicon-10K using Buffer A without Tween-20 for 8 times to produce a PDITC-activated amine-DNA solution. The PDITC activation ratio is over 90% as determined by MALDI-TOF mass spectrometry obtained after desalting the DNA product. Then, 10 mg of invertase is added to the activated DNA solution in Buffer A without Tween-20 to reach a final concentration about 5 mg/mL. The resulting solution is kept at room temperature for 48 hours. To remove un-reacted PDITC-activated amine-DNA, the solution is purified by Amicon-100K 8 times using Buffer A without Tween-20. Tween is not necessary for invertase activity; (2) 1 mg/ml invertase reaction likely finishes after 5 min; (3) 2% sucrose input produces red color at RT after ˜15 min; and (4) DNS is not effective for <0.2% sucrose.

### Example 16

### Lateral Flow Cleavage Reporters for CRISPR Diagnostics for Detection of Target Nucleic Acids from a Respiratory Virus

This example describes lateral flow cleavage reporters for CRISPR diagnostics for detection of target nucleic acids from a respiratory virus (e.g., an influenza virus). One design of the Cas reporters disclosed herein involves tethering the Cas reporter to the reaction chamber, upstream of the lateral flow test strip.

FIG. 36 shows one design of a tethered lateral flow Cas reporter. To the left is a DNA or RNA linker connecting a functional handle for chemical conjugation at the 3′ end (amine, thiol, etc.) and a biotin at the 5′ end (shown as a diamond) further connected to a FAM reporter molecule. This entire Cas reporter is conjugated to a magnetic bead and immobilized to the surface of the reaction chamber. After CRISPR-Cas cleavage reactions, the DNA/RNA linker is cleaved and the biotin/FAM reporter moiety is released.

FIG. 37 shows a workflow for CRISPR diagnostics using the tethered cleavage reporter using magnetic beads. First, CRISPR-Cas protein RNPs are incubated with target nucleic acids and magnetic beads were conjugated to the reporter. Magnetic beads are captured with a magnet, the supernatant is removed, and the sample is placed on a lateral flow strip with chase buffer.

Tethered cleavage reporters can also be used to multiplex readouts from CRISPR diagnostics. FAM-biotin and DIG-biotin reporter conjugated to magnetic beads is incubated with a Cas12 variant (SEQ ID NO: 37) for 30 minutes at 37 C in the presence or absence of target DNA (˜0.5 nM) in two separate DETECTR reactions. After the incubation period the magnetic beads are pelleted and the supernatant transferred to a PCRD lateral flow strip (Abingdon Health).

FIG. 38 shows a schematic for an enzyme-reporter system that is filtered by streptavidin-biotin before reaching the reaction chamber. The reporter structure is shown at left and includes a DNA/RNA linker connecting biotin and an enzyme. In the presence of the target (shown at top), Cas proteins cleave the linker in the Cas reaction chamber, leading to binding of biotin to the streptavidin inside of a capture chamber or on a paper strip, and enzymatic activity exhibited in a detection chamber containing the enzyme's substrate. In the absence of the target (shown at bottom), Cas proteins do not cleave the linker in the Cas reaction chamber, leading to binding of the full reporter inside of the capture chamber, and no enzyme (thus, no enzymatic activity) in the detection chamber containing the enzyme's substrate.

### Example 17

### Lateral Flow Assays for an Influenza CRISPR Diagnostic

This example describes a lateral flow assay for an influenza CRISPR diagnostic. A DNA or RNA linker is conjugated at the 5′end to biotin-dT/FAM and conjugated at the 3′ end to the substrate of a DETECTR chamber/bead, as shown in FIG. 32. These Cas reporters are inside a reaction chamber upstream of a lateral flow test strip. Cas enzymes and a sample containing target nucleic acids from an influenza virus are added, along with reagents for amplification. Amplified target nucleic acids activate transcleavage of the DNA or RNA linker by the Cas protein. The Cas protein is Cas12, Cas12a, Cas12b, Cas12c, Cas12d, Cas13, Cas13a, or Cas14. The biotin-dt/FAM reporter moiety is released and flows downstream to the test line where it binds streptavidin, as shown in FIG. 33. The biotin-dT/FAM reporter moiety is bound by anti-FAM antibody coated gold nanoparticles, which also bind to anti-rabbit antibody coated at a flow control line, thus revealing a positive test result. In the absence of target nucleic acids from an influenza virus in the sample, the Cas reporter remains immobilized to the substrate in the reaction chamber and anti-FAM antibody coated gold nanoparticles only bind to anti-rabbit antibody coated at a flow control line, thus revealing a negative test result.

### Example 18

### Multiplexed Influenza CRISPR Diagnostics

This example describes a lateral flow assay for an influenza CRISPR diagnostic. A DNA or RNA linker is conjugated at the 5′end to biotin-dT/FAM and a second DNA or RNA linker is conjugated at the 5′end to biotin-dT/ROX. Both reporters are conjugated at the 3′ end to the substrate of a DETECTR chamber/bead, as shown in FIG. 61. These Cas reporters are inside a reaction chamber upstream of a lateral flow test stripCas enzymes and a sample containing target nucleic acids from an influenza virus are added, along with reagents for amplification. Amplified target nucleic acids activate transcleavage of the DNA or RNA linker by the Cas protein. The Cas protein is Cas12, Cas12a, Cas12b, Cas12c, Cas12d, Cas13, Cas13a, or Cas14. The biotin-dt/FAM and/or the biotin-dt/ROX reporter moiety is released and flows downstream to the test line where it binds streptavidin, as shown in FIG. 33. The reporter moieties are bound by anti-FAM antibody and/or an anti-ROX antibody coated gold nanoparticles, which also bind to anti-rabbit antibody coated at a flow control line, thus revealing a positive test result. In the absence of target nucleic acids from an influenza virus in the sample, the Cas reporter remains immobilized to the substrate in the reaction chamber and anti-FAM antibody coated gold nanoparticles only bind to anti-rabbit antibody coated at a flow control line, thus revealing a negative test result.

### Example 19

### Diagnosing Influenza in Subject with a CRISPR Diagnostic

This example describes diagnosing influenza in a subject with a CRIPSR Cas diagnostic of the present disclosure. A sample is taken from a subject, such as a buccal swab or nasal swab. The subject has an undiagnosed illness. The sample is added to a CRISPR-Cas diagnostic of the present disclosure, for example, the CRISPR-Cas diagnostic of EXAMPLE 17. Guides are designed against influenza virus. The influenza virus is influenza A virus or influenza B virus. The target nucleic acid in the sample, corresponding to influenza, binds to the guide sequence, thus activating transcollateral cleavage of the Cas reporters by a Cas protein. The Cas protein is Cas12, Cas12a, Cas12b, Cas12c, Cas12d, Cas13, Cas13a, or Cas14. The CRISPR diagnostic, thus, reveals a positive result, and the subject is diagnosed with influenza.

### Example 20

### Influenza CRISPR-Cas Companion Diagnostic

This example describes an influenza CRISPR-Cas companion diagnostic of the present disclosure. A sample is taken from a subject, such as a buccal swab or nasal swab. The subject has influenza and has been prescribed and taking a flu therapeutic. The sample is added to a CRISPR-Cas diagnostic of the present disclosure, for example, the CRISPR-Cas diagnostic of EXAMPLE 17. Guides are designed against influenza virus. The influenza virus is influenza A virus or influenza B virus. The target nucleic acid in the sample, corresponding to influenza, binds to the guide sequence, thus activating transcollateral cleavage of the Cas reporters by a Cas protein. The Cas protein is Cas12, Cas12a, Cas12b, Cas12c, Cas12d, Cas13, Cas13a, or Cas14. The CRISPR diagnostic, thus, reveals a result, indicating that the flu therapeutic has not completely eliminated the influenza virus in the subject.

### Example 21

### Invertase-Nucleic Acid as a Detector Nucleic Acid

This example shows an invertase-nucleic acid as a detector nucleic for detection of a target nucleic acid from a respiratory virus (e.g., an influenza virus)in a programmable nuclease system.

FIG. 39 shows an invertase-nucleic acid used for the detection of a target nucleic acid. The invertase-nucleic acid, immobilized on a magnetic bead, is added to a sample reaction containing Cas protein, guide RNA, and a target nucleic acid. Target recognition activates the Cas protein to cleave the nucleic acid of the invertase-nucleic acid, liberating the invertase enzyme from the immobilized magnetic bead. This solution is either be transferred to the “reaction mix”, which contains sucrose and the DNS reagent and changes color from yellow to red when the invertase converts sucrose to glucose or is can be transferred to a hand-held glucometer device for a digital readout.

### Example 22

### Assay Layouts and Workflows for DETECTR Reactions

This example describes assay layouts and workflows for DETECTR reactions for detection of target nucleic acids from a respiratory virus (e.g., an influenza virus). An assay is provided that comprises separate chambers for amplification and reverse transcription versus a programmable nuclease-based detection assay. The programmable nuclease is a Cas12, Cas13, or Cas14. The sample is a biofluid collected by a swab and inserted into a swab collection reservoir. A pump drives the fluidics in the assay moving sample from chamber to chamber. A detectable signal is colorimetric, fluorescence-based, electrochemical and/or generated using an enzyme (e.g., invertase).

FIG. 40 shows one layout for a DETECTR assay. In this layout a swab collection cap seals a swab reservoir chamber. Clockwise to the swab reservoir chamber is a chamber holding the amplification reaction mix. Clockwise to the chamber holding the amplification reaction mix is a chamber holding the DETECTR reaction mix. Clockwise to this is the detection area. Clockwise to the detection area is the pH balance well. A cartridge wells cap is shown and seals all the wells containing the various reagent mixtures. The cartridge itself is shown as a square layer at the bottom of the schematic. To the right is a diagram of the instrument pipers pump which drives the fluidics in each chamber/well and is connected to the entire cartridge. Below the cartridge is a rotary valve that interfaces with the instrument. FIG. 41 shows one workflow of the various reactions in the DETECTR assay of FIG. 40. First, as shown in the top left diagram, a swab may be inserted into the 200 ul swab chamber and mixed. In the middle left diagram, the valve is rotated clockwise to the “swab chamber position” and 1 uL of sample is picked up. In the lower left diagram, the valve is rotated clockwise to the “amplification reaction mix” position and the 1 ul of sample is dispensed and mixed. In the top right diagram, 2 uL of sample is aspirated from the “amplification reaction mix”. In the top middle diagram, the valve is roated clockwise to the “DETECTR” position, the sample is dispsensed and mixed, and 20 ul of the sample is aspirated. Finally, in the bottom right diagram, the valve is rotated clockwise to the detection area position and 20 ul of the sample is dispensed. While the rotary valve is in a closed position, the sample is loaded into the swab lysis chamber and sealed using the cap. The sample is then incubated and mixed by the instrument with the lysis buffer. Following sample lysis, the rotary valve turns to align with the sample well and aspirates 2 to 4 μL of sample. The rotary valve then turns to align with the amplification chamber, where the sample is mixed with the amplification mixture. The sample is then aspirated, and the rotary valve rotates to the DETECTR chamber. In the DETECTR chamber, the sample mixes with the DETECTR mix. Actuation of the pipette pump mixes the reaction mixtures. The process may then be repeated from the amplification chamber to a second DETECTR chamber.

FIG. 42 shows a modification of the workflow shown in FIG. 41 that is also consistent with the methods and systems of the present disclosure. At left is the diagram shown at the top right of FIG. 41. At right is the modified diagram in which there is a first amplification chamber counterclockwise to the swab lysis chamber and a second amplification chamber clockwise to the swab lysis chamber. Additionally, clockwise to amplification chamber #2 are two sets, or “duplex”, DETECTR chambers labeled “Duplex DETECTR Chambers #2” and “Duplex DETECTR Chambers #1”, respectively. FIG. 43 shows breakdown of the workflow for the modified layout shown in FIG. 42. Specifically, from the swab lysis chamber, which holds 200 ul of sample, 20 ul of the sample can be moved to amplification chamber #1 and 20 ul of the sample can be moved to amplification chamber #2. After amplification in amplification chamber #1, 20 ul of the sample can be moved to Duplex DETECTR Chambers #1a and 20 ul of the sample can be moved to Duplex DETECTR Chambers # 1 b. Additionally, after amplification in amplification chamber #2, 20 ul of the sample can be moved to Duplex DETECTR Chambers #2a and 20 ul of the sample can be moved to Duplex DETECTR Chambers #2b.

FIG. 44 shows the modifications to the cartridge illustrated in FIG. 42 and FIG. 43. FIG. 45 shows a top down view of the cartridge of FIG. 44. This layout and workflow has a replicate in comparison to the layout and workflow of FIGS. 40-41. FIG. 46 shows a layout for a two-pot DETECTR assay. Shown at top is a pneumatic pump, which interfaces with the cartridge. Shown at middle is a top down view of the cartridge showing a top layer with reservoirs. Shown at bottom is a sliding valve containing the sample and arrows pointing to the lysis chamber at left, following by amplification chambers to the right, and DETECT chambers further to the right. FIG. 57 shows a schematic of the sliding valve device. FIG. 58 shows a layout and workflow for a sliding valve device. In the initial closed position (i.), the sample is loaded into the sample well and lysed. The sliding valve is then actuated by the instrument, and samples are loaded into each of the channels using the pipette pump, which dispenses the appropriate volume into the channel (ii.). The sample is delivered to the amplification chambers by actuating the sliding valve and mixed with the pipette pump (iii.). Samples from the amplification chamber are aspirated into each channel (iv.) and then dispensed and mixed into each DETECTR chamber (v.) by actuating the sliding valve and pipette pump.

### Example 23

### DETECTR Assays Versus PCR-Based Detection

This example describes a comparison of the DETECTR assays disclosed herein to the gold standard: PCR-based methods of detecting a target nucleic acid. Samples were either used as a crude prep for DETECTR assays (only lysed) or purified (lysed, bound, washed, and eluted) for PCR-based methods of detection. A DETECTR assay using a programmable nuclease (e.g., a Cas protein) is carried out on the crude sample. The programmable nuclease is activated by the target nucleic acid in a sample to which it binds via a reverse complementary guide RNA. The activated programmable nuclease indiscriminately cleaves a reporter generating a fluorescent detectable signal. Standard PCR-based methods were used to also detect the target nucleic acids in the sample.

FIG. 47 shows a comparison of the DETECTR assays disclosed herein to the gold standard PCR-based method of detecting a target nucleic acid. Shown is a flow chart showing a gradient of sample prep evaluation from crude (left) to pure (right). Sample prep steps that take a crude sample to a pure sample include lysis, binding, washing, and eluting. DETECTR assays disclosed herein may only need the sample prep step of lysis, yielding a crude sample. On the other hand, PCR-based methods can require lysis, binding, washing, and elution, yielding a very pure sample. The DETECTR assay disclosed herein can identify target nucleic acids from respiratory viruses (e.g., an influenza virus) just as well as gold standard PCR-based methods of detection.

### Example 24

### Cas13a Detection of DNA

This example describes Cas13a detection of target DNA. Cas13a was used to detect a target RT-LAMP DNA amplicon from Influenza A RNA. FIG. 48A shows a schematic The RT-LAMP reaction was performed at 55° C. for 30 minutes with a starting RNA concentration of 10,000 viral genome copies or 0 viral genome copies, as a control. Two different primer sets showed the same results (FIG. 48B and FIG. 48C). After completion of the RT-LAMP reaction, 1 μL of amplicon was added to a 20 μL Cas13a detection reaction. On-target and off-target crRNAs were used to show specific detection by Cas13a at 37° C. of the RT-LAMP DNA amplicon.

FIG. 48A shows a schematic of the workflow including providing DNA/RNA, LAMP/RT-LAMP, and Cas13a detection. FIG. 48B shows Cas13a specific detection of target RT-LAMP DNA amplicon with a first primer set as measured by background subtracted fluorescence on the y-axis. On-target crRNA results are shown by the darker bars and off-target crRNA control results are shown in lighter bars. A starting RNA concentration of 10,000 viral genome copies is shown in the left two bars and 0 viral genome copies (negative control) is shown in the right two bars. FIG. 48C shows Cas13a specific detection of target RT-LAMP DNA amplicon with a second primer set as measured by background subtracted fluorescence on the y-axis. On-target crRNA results are shown by the darker bars and off-target crRNA control results are shown in lighter bars. A starting RNA concentration of 10,000 viral genome copies is shown in the left two bars and 0 viral genome copies (negative control) is shown in the right two bars.

Cas13a recognized target ssDNA and target RNA. FIG. 49A shows a Cas13 detection assay using 2.5 nM RNA, single-stranded DNA (ssDNA), or double-stranded (dsDNA) as target nucleic acids, where detection was measured by fluorescence for each of the target nucleic acids tested. The reaction was performed at 37° C. for 20 minutes with both RNA-FQ (RNA-fluorescence quenched reporter) and DNA-FQ reporter substrates. Results showed that Cas13 initiates trans-cleavage activity for RNA-FQ for both target RNA and target ssDNA. Data was normalized to max fluorescence signal for each reporter substrate. FIG. 49B shows Cas12 detection assay using 2.5 nM RNA, ssDNA, and dsDNA as target nucleic acids, where detection was measured by fluorescence for each of the target nucleic acids tested. Reactions were performed at 37° C. for 20 minutes with both RNA-FQ and DNA-FQ reporter substrates. Results supported the previously established preference for Cas12 for either target ssDNA or target dsDNA and specificity for DNA-FQ. Data was normalized to max fluorescence signal for each reporter substrate. FIG. 49C shows the performance of Cas13 and Cas12 on target RNA, target ssDNA, and target dsDNA at various concentrations, where detection was measured by fluorescence for each of the target nucleic acids tested. Reactions were performed at 37° C. for 90 minutes with both RNA-FQ and DNA-FQ reporter substrates. Data was normalized to max fluorescence signal for each reporter substrate. Results indicated picomolar sensitivity of Cas13 for target ssDNA.

Cas13a trans-cleavage activity was found to be specific for RNA reporters when targeting target ssDNA. FIG. 50 shows an LbuCas13a (SEQ ID NO: 131) detection assay using 2.5 nM target ssDNA with 170 nM of various reporter substrates, wherein detection was measured by fluorescence for each of the reporter substrates tested. A single RNA-FQ reporter substrate (rep01—FAM-U5) was tested and 13 DNA-FQ reporter substrates were tested. TABLE 12 below shows the sequence of each of the reporters tested.

TABLE 12
Reporter Sequences

| | SEQ | |
|---|---|---|
| Reporter | ID | |
| ID | NO: | Sequence |
| rep01 | 1 | /56-FAM/rUrUrUrUrU/3IABkFQ/ |
| rep08 | 382 | /56-FAM/AAAAA/3IABkFQ/ |
| rep09 | 383 | /56-FAM/CCCCC/3IABkFQ/ |
| rep10 | 384 | /56-FAM/GGGGG/3IABkFQ/ |
| rep11 | 385 | /56-FAM/TTTTT/3IABkFQ/ |
| rep12 | 386 | /56-FAM/TTATTA/3IABkFQ/ |
| rep13 | 9 | /56-FAM/TTATTATT/3IABkFQ/ |
| rep14 | 387 | /56-FAM/ATTATTATTA/3IABkFQ/ |
| rep15 | 10 | /56-FAM/TTTTTT/3IABkFQ/ |
| rep16 | 388 | /56-FAM/TTTTTTT/3IABkFQ/ |
| rep17 | 12 | /56-FAM/TTTTTTTTTT/3IABkFQ/ |
| rep18 | 389 | /56-FAM/TTTTTTTTTTT/3IABkFQ/ |
| rep19 | 13 | /56-FAM/TTTTTTTTTTTT/3IABkFQ/ |
| rep30 | 390 | /FAM/CCGGCAGCCATAACGCCGTGAATACGTTCTGCCGG/BHQ1/ |

Results indicated that Cas13 trans-cleavage was specific for RNA reporters, even when activated by target ssDNA.

Multiple Cas13 family members detected target ssDNA. FIG. 51A shows the results of Cas13 detection assays for LbuCas13a (SEQ ID NO: 131) and LwaCas13a (SEQ ID NO: 137) using 10 nM or 0 nM of target RNA, where detection was measured by fluorescence resulting from cleavage of reporters over time. Three target RNAs encoding different sequences were evaluated with corresponding gRNAs. Results showed similar detection of all three target nucleic acids for both Cas13 family members. FIG. 51B shows the results of Cas13 detection assays for LbuCas13a and LwaCas13a using 10 nM or 0 nM of target ssDNA, where detection was measured by fluorescence resulting from cleavage of reporters over time. Three target DNA and their corresponding gRNAs, with the same sequence as the target RNAs, were evaluated. Results showed Cas13 family preferences in target ssDNA recognition, with LbuCas13a exhibiting faster detection for some target nucleic acids and LwaCas13a exhibiting faster detection for other targets

Cas13 detection of target ssDNA was robust at multiple pH values. FIG. 52 shows LbuCas13a detection assay using 1 nM target RNA (at left) or target ssDNA (at right) in buffers with various pH values ranging from 6.8 to 8.2. Reactions were performed at 37° C. for 20 minutes with RNA-FQ reporter substrates. Results indicated enhanced Cas13 RNA detection at buffers with a higher pH (7.9 to 8.2), whereas Cas13 ssDNA detection was consistent across pH conditions (6.8 to 8.2).

Cas13 preferences for target ssDNA were found to be distinct from preferences for target RNA. FIG. 53A shows guide RNAs (gRNAs) tiled along a target sequence at 1 nucleotide intervals. FIG. 53B shows LbuCas13a (SEQ ID NO: 131) detection assays using 0.1 nM RNA or 2 nM target ssDNA with gRNAs tiled at 1 nucleotide intervals and an off-target gRNA. Guide RNAs were ranked by their position along the sequence of the target nucleic acid. FIG. 53C shows data from FIG. 53B ranked by performance of target ssDNA. Results showed that gRNA performance on target ssDNA did not correlate with the performance of the same gRNAs on RNA. FIG. 53D shows performance of gRNAs for each nucleotide on a 3′ end of a target RNA. Results indicated that there are high performing gRNAs on target RNAs regardless of target nucleotide identity at this position. FIG. 53E shows performance of gRNAs for each nucleotide on a 3′ end of a target ssDNA. Results indicated that a G in the target at this position performed worse than other gRNAs.

Cas13a detected target DNA generated by nucleic acid amplification methods (PCR, LAMP). FIG. 54A shows LbuCas13a (SEQ ID NO: 131) detection assays using 1 μL of target DNA amplicon from various LAMP isothermal nucleic acid amplification reactions. LAMP conditions tested included 6-primer with both loop-forward (LF) and loop-reverse (LB), asymmetric LAMP with LF only, and asymmetric LAMP with LB only. All tested LAMP reactions generated an LbuCas13a compatible target DNA. FIG. 54B shows LbuCas13a (SEQ ID NO: 131) detection assays using various amounts of PCR reaction as a target DNA. Results indicated that PCR generated enough target ssDNA to enable Cas13 detection.

### Example 25

### Layouts and Workflows for DETECTR Reactions

This example describes assay layouts and workflows for DETECTR reactions. An assay is provided that comprises separate chambers for amplification and reverse transcription versus a programmable nuclease-based detection assay. The programmable nuclease is a Cas12, Cas13, or Cas14. The sample is a biofluid collected by a swab and inserted into a swab collection reservoir. The biofluid sample is tested for the presence of a target nucleic acid from an influenza virus. A pump drives the fluidics in the assay moving sample from chamber to chamber. A detectable signal is colorimetric, fluorescence-based, electrochemical and/or generated using an enzyme (e.g., invertase).

FIG. 55A shows a schematic of a pneumatic valve device. A pipette pump aspirates and dispenses samples. An air manifold is connected to a pneumatic pump to open and close the normally closed valve. The pneumatic device moves fluid from one position to the next and isolates unused parts of the system. The pneumatic design has reduced channel cross talk compared to other devices. FIG. 55B shows a schematic of a cartridge for use in the pneumatic valve device. The normally closed valves (one such valve is indicated by an arrow) comprise an elastomeric seal on top of the channel to isolate each chamber from the rest of the system when the chamber is not in use. The pneumatic pump uses air to open and close the valve as needed to move fluid to the necessary chambers within the cartridge. The cartridge is able to incorporate multiple different sample media. The cartridge can accommodate lysis buffer volumes of 200 μL and perform incubation steps, for example, a 10 minute incubation. The cartridge accommodates aspiration of two 2 μL samples from up to four amplification chambers. The two samples can be dispensed into the corresponding detection chambers with limited cross contamination between amplification chambers or detection chambers. The cartridge accommodates transfer of 1-2 μL of lysed sample from the sample input chamber to an amplification chamber. The cartridge may comprise up to four amplification chambers, with two detection chambers per amplification chamber, for a total of up to eight detection chambers. Each DETECTR chamber may be imaged, for example by a spectrometer. As shown in FIG. 55 and illustrated in FIG. 56, the cartridge may have two amplification chambers and two detection chambers per amplification chamber.

FIG. 56 shows a valve circuitry layout for the pneumatic valve device. The biofluid sample is placed in the sample well while all valves are closed, as shown at (i.). The sample is lysed in the sample well. The lysed sample is moved from the sample chamber to a second chamber by opening the first quake valve, as shown at (ii.), and aspirating the sample using the pipette pump. The sample is then moved to the first amplification chamber by closing the first quake valve and opening a second quake valve, as shown at (iii.) where it is mixed with the amplification mixture. After the sample is mixed with the amplification mixture, it is moved to a subsequent chamber by closing the second quake valve and opening a third quake valve, as shown at (iv). The sample is moved to the detection chamber by closing the quick third valve and opening a quick fourth valve, as shown at (v). The detection chamber comprises the programmable nuclease. If a target nucleic acid is present in the sample, a detectable signal may be produced. The detectable signal may be imaged in the detection chamber. The sample can be moved through a different series of chambers by opening and closing a different series of quake valves, as shown at (vi). Actuation of individual valves in the desired chamber series prevents cross contamination between channels.

FIG. 59 shows a schematic of the top layer of a cartridge of a pneumatic valve device of the present disclosure, highlighting suitable dimensions. The schematic shows one cartridge that is 2 inches by 1.5 inches. FIG. 60 shows a schematic of a modified top layer of a cartridge of a pneumatic valve device of the present disclosure adapted for electrochemical dimension. In this schematic, three lines are shown in the detection chambers (4 chambers at the very right). These three lines represent wiring (or “metal leads”), which is co-molded, 3D-printed, or manually assmpled in the disposable cartridge to form a three-electrode system. Electrodes are termed as working, counter, and reference. Electrodes can also be screen-printed on the cartridges. Metals used can be carbon, gold, platinum, or silver.

### Example 26

### Primer Design for Combined LAMP and DETECTR Reactions

This example describes primer design for combined LAMP and DETECTR reactions for amplification and detection of a target nucleic acid, as provided herein. Strategies for designing primers for use in combined LAMP and DETECTR reactions were tested and evaluated for multiple target nucleic acids. From these experiments, a set of design guidelines was determined to facilitate combined LAMP and DETECTR reactions for DNA nucleic acid targets or RT-LAMP and DETECTR reactions for RNA nucleic acid targets.

FIG. 61 shows a scheme for designing primers for loop mediated isothermal amplification (LAMP) of a target nucleic acid sequence. LAMP generates concatemer amplicons, comprising the target nucleic acid sequence, that form from nucleic acid loops during amplification. To generate the loops, LAMP may use from four to six primers, including the forward outer primer, the backward outer primer, the forward inner primer, the backward inner primer, optionally a loop forward primer, and optionally a loop backward primer.

FIG. 62 shows schematics of exemplary configurations of various regions of the nucleic acid sequence that correspond to or anneal LAMP primers, guide RNA sequences, protospacer-adjacent motif (PAM) or protospacer flanking site (PFS), and target nucleic acid sequences for amplification and detection by LAMP and DETECTR.

FIG. 62A shows a schematic of an exemplary arrangement of the guide RNA (gRNA) with respect to the various regions of the nucleic acid sequence that correspond to or anneal LAMP primers. In this arrangement, the guide RNA is reverse complementary to a sequence of the target nucleic acid, which is between an F1c region and a B1 region.

FIG. 62B shows a schematic of an exemplary arrangement of the guide RNA sequence with respect to the various regions of the nucleic acid sequence that correspond to or anneal LAMP primers. In this arrangement, the guide RNA is partially reverse complementary to a sequence of the target nucleic acid, which is between an F1c region and a B1 region. For example, the target nucleic acid comprises a sequence between an F1c region and a B1 region that is reverse complementary to at least 60% of a guide nucleic acid. In this arrangement, the guide RNA is not reverse complementary to the forward inner primer or the backward inner primer shown in FIG. 61.

FIG. 62C shows a schematic of an exemplary arrangement of the guide RNA with respect to the various regions of the nucleic acid sequence that correspond to or anneal LAMP primers. In this arrangement, the guide RNA hybridizes to a sequence of the target nucleic acid, which is within the loop region between the B1 region and the B2 region. The primer sequences do not contain and are not reverse complementary to the PAM or PFS.

FIG. 62D shows a schematic of an exemplary arrangement of the guide RNA with respect to the various regions of the nucleic acid sequence that correspond to or anneal LAMP primers. In this arrangement, the guide RNA hybridizes to a sequence of the target nucleic acid, which is within the loop region between the F2c region and F1c region. The primer sequences do not contain and are not reverse complementary to the PAM or PFS.

Primer sets and guide RNAs for combined LAMP and DETECTR reactions were tested for their sensitivity and specificity to detect the presence of a target nucleic acid in a sample. DETECTR signal, measured as raw fluorescence, was measured for each LAMP primer set with each of three guide RNAs designed for the specific LAMP primer set. DETECTR signal was measured in a sample containing 10000 copies of a target nucleic acid sequence and a sample containing zero copies of a target nucleic acid sequence (negative control) for each LAMP primer and guide RNA pair.

FIG. 63 shows schematics of exemplary configurations of various regions of the nucleic acid sequence that correspond to or anneal LAMP primers, guide RNA sequences, protospacer-adjacent motif (PAM) or protospacer flanking site (PFS), and target nucleic acid sequences for combined LAMP and DETECTR for amplification and detection, respectively. At the right, the schematics also show corresponding fluorescence data using the and guide RNA sequences to detect the presence of a target nucleic acid sequence, following amplification of the target nucleic acid using the LAMP amplification, where a fluorescence signal is the output of the DETECTR reaction and indicates presence of the target nucleic acid. Sequences and arrangements of the regions that correspond to or anneal LAMP primers, guide RNA sequences, protospacer-adjacent motif (PAM) or protospacer flanking site (PFS), and target nucleic acid sequences are illustrated in FIG. 64A-FIG. 64C. Three exemplary guide RNAs (gRNA1 (SEQ ID NO: 271), gRNA2 (SEQ ID NO: 272), and gRNA3 (SEQ ID NO: 273)) were tested in each primer configuration. Fluorescence signal from the DETECTR reactions, indicative of detection of a target nucleic acid, measured for each of the three guide RNAs was compared for two samples, one containing the target nucleic acid sequence (1000 genome copies per reaction) and a negative control (0 genome copies per reaction) that does not contain the target nucleic acid sequence. Sequences of the gRNAs and the primers are shown below in TABLE 13.

TABLE 13
Exemplary LAMP Primer and DETECTR gRNA Sets

| SEQ ID NO: | Name | Sequence |
|---|---|---|
| SEQ ID NO: 211 | IAV-MP-set5-F3 | GCGAAAGCAGGTAGATATTGA |
| SEQ ID NO: 259 | IAV-MP-set5-F2 | ATGAGTCTTCTAACCGAGGT |
| SEQ ID NO: 215 | IAV-MP-set5-LF | TGACGGGACGATAGAGAGAA |
| SEQ ID NO: 260 | IAV-MP-set5-F1c | TTCAAGTCTCTGCGCGATCTC |
| SEQ ID NO: 261 | IAV-MP-set5-B1c | TTGAGGCTCTCATGGAATGGC |
| SEQ ID NO: 216 | IAV-MP-set5-LB | ACAAGACCAATCCTGTCACC |
| SEQ ID NO: 262 | IAV-MP-set5-B2 | AGCGTGAACACAAATCCTAA |
| SEQ ID NO: 212 | IAV-MP-set5-B3 | CATTCCCATTGAGGGCATT |
| SEQ ID NO: 220 | IAV-MP-set8-F3 | TCTTCTAACCGAGGTCGAA |
| SEQ ID NO: 263 | IAV-MP-set8-F2 | GAAGATGTCTTTGCAGGGAA |
| SEQ ID NO: 224 | IAV-MP-set8-LF | ATTCCATGAGAGCCTCAAGATC |
| SEQ ID NO: 264 | IAV-MP-set8-F1c | TCAGAGGTGACAGGATTGGTCT |
| SEQ ID NO: 265 | IAV-MP-set8-B1c | TTGTGTTCACGCTCACCGTG |
| SEQ ID NO: 225 | IAV-MP-set8-LB | GAGGACTGCAGCGTAGAC |
| SEQ ID NO: 212 | IAV-MP-set8-B2 | CATTCCCATTGAGGGCATT |
| SEQ ID NO: 221 | IAV-MP-set8-B3 | CTGCTCTGTCCATGTTGTT |
| SEQ ID NO: 266 | IAV-MP-set1-F3 | GACTTGAAGATGTCTTTGCA |
| SEQ ID NO: 267 | IAV-MP-set1-F2 | CAGATCTTGAGGCTCTC |
| SEQ ID NO: 268 | lAV-MP-set1-LF | GTCTTGTCTTGTCTTTAGCCA |
| SEQ ID NO: 269 | lAV-MP-set1-F1c | TTAGTCAGAGGTGACAGGATTG |
| SEQ ID NO: 265 | lAV-MP-set1-B1c | TTGTGTTCACGCTCACCGTG |
| SEQ ID NO: 198 | lAV-MP-set1-LB | CAGTGAGCGAGGACTG |
| SEQ ID NO: 270 | IAV-MP-set1-B2 | TTTGGACAAAGCGTCTACG |
| SEQ ID NO: 194 | IAV-MP-set1-B3 | TGTTGTTTGGGTCCCCATT |
| SEQ ID NO: 271 | gRNA1 | UUUGUGUUCACGCUCACCGUGCCC |
| SEQ ID NO: 272 | gRNA2 | UUUAGCCAUUCCAUGAGAGCCUCA |
| SEQ ID NO: 273 | gRNA3 | UUUGGACAAAGCGUCUACGCUGCA |

FIG. 63A shows a schematic of an arrangement of various regions of the nucleic acid sequence that correspond to or anneal LAMP primers (SEQ ID NO: 211, SEQ ID NO: 212, SEQ ID NO: 215, SEQ ID NO: 216, and SEQ ID NO: 259-SEQ ID NO: 262) and positions of three guide RNAs (gRNA1 (SEQ ID NO: 271), gRNA2 (SEQ ID NO: 272), and gRNA3 (SEQ ID NO: 273)) relative to the LAMP primers (at left). gRNA1 partially overlaps with the B2c region and is, thus, reverse complementary to a portion of the B2 region. gRNA2 overlaps with the B1 region and is, thus, reverse complementary to the B1c region. gRNA3 partially overlaps with the B3 region and partially overlaps with the B2 region and is, thus, partially reverse complementary to the B3c region and partially reverse complementary to the B2c region. The complementary regions (B1c, B2c, B3c, F1c, F2c, and F3c) are not depicted, but correspond to the regions shown in FIG. 61. At right is a graph of fluorescence from the DETECTR reaction in the presence of 10,000 genome copies (before amplification) of the target nucleic acid or 0 genome copies of the target nucleic acid. DETECTR reactions with gRNA1 and gRNA3 exhibited low fluorescence intensity, indicating low to no detection of the target nucleic acid (right). gRNA2 produced a fluorescent signal independent of the presence of the target nucleic acid due to hybridization of gRNA2 with the B1c region of the BIP and self-activation of the guide RNA. and Cas cleavage activity. Hybridization of gRNA2 with the BIP may further lead to amplification of a non-target sequence due to the formation of a primer dimer.

FIG. 63B shows a schematic of an arrangement of various regions of the nucleic acid sequence that correspond to or anneal LAMP primers (SEQ ID NO: 212, SEQ ID NO: 220, SEQ ID NO: 221, SEQ ID NO: 224, SEQ ID NO: 225, SEQ ID NO: 263-SEQ ID NO: 265) and positions of three guide RNAs (gRNA1 (SEQ ID NO: 271), gRNA2 (SEQ ID NO: 272), and gRNA3 (SEQ ID NO: 273)) relative to the LAMP primers (at left). gRNA1 overlaps with the B1c region and is, thus, reverse complementary to the B1 region. gRNA2 overlaps with the LF region and is, thus, reverse complementary to the LFc region. gRNA 3 partially overlaps with the B2 region and partially overlaps with the LBc region and is, thus, partially reverse complementary to the B2c region and is partially reverse complementary to the LB region. At right is a graph of fluorescence from the DETECTR reaction in the presence of 10,000 genome copies of the target nucleic acid or 0 genome copies of the target nucleic acid. All three guide RNAs detected the presence of the target nucleic acid in DETECTR reactions, as evidenced by a high fluorescence signal in the presence of the target nucleic acid (right). gRNA1 also produced a non-specific fluorescent signal in the absence of the target nucleic acid due to primer-dimer formation with the BIP. gRNA2 and gRNA3 did not produce a substantial non-specific fluorescent signal.

FIG. 63C shows a schematic of an arrangement of various regions of the nucleic acid sequence that correspond to or anneal LAMP primers (SEQ ID NO: 194, SEQ ID NO: 198, SEQ ID NO: 265-SEQ ID NO: 270) and positions of three guide RNAs (gRNA1 (SEQ ID NO: 271), gRNA2 (SEQ ID NO: 272), and gRNA3 (SEQ ID NO: 273)) relative to the LAMP primers (at left). gRNA1 overlaps with the B1c region and is, thus, reverse complementary to the B1 region. gRNA2 partially overlaps with the LF region and partially overlaps with the F2c region and is, thus, partially reverse complementary to the LFc region and partially reverse complementary to the F2 region. gRNA3 overlaps with the B2 and is, thus, reverse complementary to the B2c region. At right is a graph of fluorescence from the DETECTR reaction in the presence of 10,000 genome copies of the target nucleic acid or 0 genome copies of the target nucleic acid. gRNA2 and gRNA3 specifically detected the presence of the target nucleic acid in DETECTR reactions, as evidenced by a high fluorescence signal in the presence of the target nucleic acid and low fluorescence signal in the absence of the target nucleic acid (right). gRNA1 detected the presence of the target nucleic acid in a DETECTR reaction but also non-specifically produced a fluorescence signal in the absence of the target nucleic acid due to primer-dimer formation with the BIP, as evidenced by a high fluorescence signal in the presence of the target nucleic acid and a moderate fluorescence signal in the absence of the target nucleic acid.

### Example 27

### Detection of a Target Nucleic Acid with Combined LAMP and DETECTR Reactions

This example describes detection of a target nucleic acid with combined LAMP and DETECTR reactions. Ten LAMP primer sets (#1-#10) for use in RT-LAMP assays were tested for sensitivity and specificity for samples containing a target nucleic acid sequence. Detection following RT-LAMP amplification was performed using either SYTO 9 detection or DETECTR. The sequences of the LAMP primers in each primer set are provided in TABLE 14.

TABLE 14
LAMP Primers for RT-LAMP Amplification and Detection

| SEQ ID NO: | Primer Name | Primer Set | Sequence |
|---|---|---|---|
| SEQ ID NO: 148 | F3 RSV-A- | #1 | TGGAACAAGTTGTGGAGG |
| | set13 | | |
| SEQ ID NO: 149 | B3 RSV-A- | #1 | TGCAGCATCATATAGATCTTGA |
| | set13 | | |
| SEQ ID NO: 150 | FIP RSV-A- | #1 | TAGTGATGCTTTTGGGTTGTTCAAT |
| | set13 | | TGTATGAGTATGCTCAAAAATTGG |
| SEQ ID NO: 151 | BIP RSV-A- | #1 | GTGTAGTATTGGGCAATGCTGCTC |
| | set13 | | CTTGGTGTACCTCTGT |
| SEQ ID NO: 152 | LF RSV-A- | #1 | TATGGTAGAATCCTGCTTCTCC |
| | set13 | | |
| SEQ ID NO: 153 | LB RSV-A- | #1 | TGGCCTAGGCATAATGGGAGA |
| | set13 | | |
| SEQ ID NO: 154 | F3 RSV-A- | #2 | AACAAGTTGTGGAGGTGTA |
| | set14 | | |
| SEQ ID NO: 155 | B3 RSV-A- | #2 | CCATTTTCTTTGAGTTGTTCAG |
| | set14 | | |
| SEQ ID NO: 156 | FIP RSV-A- | #2 | TAGTGATGCTTTTGGGTTGTTCAA |
| | set14 | | GAGTATGCTCAAAAATTGGGTG |
| SEQ ID NO: 157 | BIP RSV-A- | #2 | GTATTGGGCAATGCTGCTGGCATA |
| | set14 | | TAGATCTTGATTCCTTGGTG |
| SEQ ID NO: 158 | LF RSV-A- | #2 | ATATGGTAGAATCCTGCTTCTC |
| | set14 | | |
| SEQ ID NO: 159 | LB RSV-A- | #2 | CCTAGGCATAATGGGAGAATAC |
| | set14 | | |
| SEQ ID NO: 154 | F3 RSV-A- | #3 | AACAAGTTGTGGAGGTGTA |
| | set15 | | |
| SEQ ID NO: 155 | B3 RSV-A- | #3 | CCATTTTCTTTGAGTTGTTCAG |
| | set15 | | |
| SEQ ID NO: 160 | FIP RSV-A- | #3 | ATAGTGATGCTTTTGGGTTGTTCA |
| | set15 | | AGTATGCTCAAAAATTGGGTG |
| SEQ ID NO: 161 | BIP RSV-A- | #3 | GCTGCTGGCCTAGGCATAATGCAT |
| | set15 | | CATATAGATCTTGATTCCTT |
| SEQ ID NO: 380 | LF RSV-A- | #3 | TATATGGTAGAATCCTGCTTCTC |
| | set15 | | |
| SEQ ID NO: 162 | LB RSV-A- | #3 | GGGAGAATACAGAGGTACAC |
| | set15 | | |
| SEQ ID NO: 163 | F3 RSV-A- | #4 | GGGTCTTAGCAAAATCAGTT |
| | set16 | | |
| SEQ ID NO: 149 | B3 RSV-A- | #4 | TGCAGCATCATATAGATCTTGA |
| | set16 | | |
| SEQ ID NO: 164 | FIP RSV-A- | #4 | GAATCCTGCTTCTCCACCCAATTG |
| | set16 | | ACACGCTAGTGTACAAGC |
| SEQ ID NO: 151 | BIP RSV-A- | #4 | GTGTAGTATTGGGCAATGCTGCTC |
| | set16 | | CTTGGTGTACCTCTGT |
| SEQ ID NO: 165 | LF RSV-A- | #4 | CCTCCACAACTTGTTCCATTTCT |
| | set16 | | |
| SEQ ID NO: 166 | LB RSV-A- | #4 | TGGCCTAGGCATAATGGGAG |
| | set16 | | |
| SEQ ID NO: 167 | F3 RSV-A- | #5 | AAGCAGAAATGGAACAAGTT |
| | set17 | | |
| SEQ ID NO: 155 | B3 RSV-A- | #5 | CCATTTTCTTTGAGTTGTTCAG |
| | set17 | | |
| SEQ ID NO: 168 | FIP RSV-A- | #5 | TAGTGATGCTTTTGGGTTGTTCAGT |
| | set1l7 | | GGAGGTGTATGAGTATGC |
| SEQ ID NO: 169 | BIP RSV-A- | #5 | GTAGTATTGGGCAATGCTGCTGAT |
| | set17 | | ATAGATCTTGATTCCTTGGTG |
| SEQ ID NO: 170 | LF RSV-A- | #5 | TGCTTCTCCACCCAATTTTTGA |
| | set17 | | |
| SEQ ID NO: 171 | LB RSV-A- | #5 | GCCTAGGCATAATGGGAGAATAC |
| | set17 | | |
| SEQ ID NO: 163 | F3 RSV-A- | #6 | GGGTCTTAGCAAAATCAGTT |
| | set18 | | |
| SEQ ID NO: 149 | B3 RSV-A- | #6 | TGCAGCATCATATAGATCTTGA |
| | set18 | | |
| SEQ ID NO: 172 | FIP RSV-A- | #6 | GAATCCTGCTTCTCCACCCAGACA |
| | set18 | | CGCTAGTGTACAAGC |
| SEQ ID NO: 151 | BIP RSV-A- | #6 | GTGTAGTATTGGGCAATGCTGCTC |
| | set18 | | CTTGGTGTACCTCTGT |
| SEQ ID NO: 165 | LF RSV-A- | #6 | CCTCCACAACTTGTTCCATTTCT |
| | set18 | | |
| SEQ ID NO: 166 | LB RSV-A- | #6 | TGGCCTAGGCATAATGGGAG |
| | set18 | | |
| SEQ ID NO: 173 | F3 RSV-A- | #7 | TACACAGCTGCTGTTCAA |
| | set19 | | |
| SEQ ID NO: 174 | B3 RSV-A- | #7 | GGTAAATTTGCTGGGCATT |
| | set19 | | |
| SEQ ID NO: 175 | FIP RSV-A- | #7 | TTGGAACATGGGCACCCATAAATG |
| | set19 | | TCCTAGAAAAAGACGATG |
| SEQ ID NO: 176 | BIP RSV-A- | #7 | CTAGTGAAACAAATATCCACACCC |
| | set19 | | AGCACTGCACTTCTTGAGTT |
| SEQ ID NO: 177 | LF RSV-A- | #7 | TTGTAAGTGATGCAGGAT |
| | set19 | | |
| SEQ ID NO: 178 | LB RSV-A- | #7 | AGGGACCCTCATTAAGAGTCATG |
| | set19 | | |
| SEQ ID NO: 179 | F3 RSV-A- | #8 | ATACACAGCTGCTGTTCA |
| | set20 | | |
| SEQ ID NO: 174 | B3 RSV-A- | #8 | GGTAAATTTGCTGGGCATT |
| | set20 | | |
| SEQ ID NO: 180 | FIP RSV-A- | #8 | TCTGCTGGCATGGATGATTGAATG |
| | set20 | | TCCTAGAAAAAGACGATG |
| SEQ ID NO: 176 | BIP RSV-A- | #8 | CTAGTGAAACAAATATCCACACCC |
| | set20 | | AGCACTGCACTTCTTGAGTT |
| SEQ ID NO: 181 | LF RSV-A- | #8 | CCCATATTGTAAGTGATGCAGGAT |
| | set20 | | |
| SEQ ID NO: 182 | LB RSV-A- | #8 | AGGGACCCTCATTAAGAGTCAT |
| | set20 | | |
| SEQ ID NO: 179 | F3 RSV-A- | #9 | ATACACAGCTGCTGTTCA |
| | set21 | | |
| SEQ ID NO: 183 | B3 RSV-A- | #9 | TGGTAAATTTGCTGGGCAT |
| | set21 | | |
| SEQ ID NO: 180 | FIP RSV-A- | #9 | TCTGCTGGCATGGATGATTGAATG |
| | set21 | | TCCTAGAAAAAGACGATG |
| SEQ ID NO: 184 | BIP RSV-A- | #9 | TGAAACAAATATCCACACCCAAGG |
| | set21 | | GCACTGCACTTCTTGAGTT |
| SEQ ID NO: 185 | LF RSV-A- | #9 | CCATATTGTAAGTGATGCAGGAT |
| | set21 | | |
| SEQ ID NO: 186 | LB RSV-A- | #9 | GACCCTCATTAAGAGTCATGAT |
| | set21 | | |
| SEQ ID NO: 187 | F3 RSV-A- | #10 | AACATACGTGAACAAACTTCA |
| | set22 | | |
| SEQ ID NO: 188 | B3 RSV-A- | #10 | GCACATATGGTAAATTTGCTGG |
| | set22 | | |
| SEQ ID NO: 189 | FIP RSV-A- | #10 | ACCCATATTGTAAGTGATGCAGGA |
| | set22 | | TAGGGCTCCACATACACAG |
| SEQ ID NO: 190 | BIP RSV-A- | #10 | CTAGTGAAACAAATATCCACACCC |
| | set22 | | AAGCACTGCACTTCTTGAG |
| SEQ ID NO: 191 | LF RSV-A- | #10 | TTTCTAGGACATTGTATTGAACAG |
| | set22 | | C |
| SEQ ID NO: 192 | LB RSV-A- | #10 | GGGACCCTCATTAAGAGTCATG |
| | set22 | | |

FIG. 65 shows the times to result of a reverse-transcription LAMP (RT-LAMP) reaction detected using a DNA binding dye. LAMP amplification, measured by an increase in SYTO 9 fluorescence, was observed over time, and time to result was determined as the time to reach half maximum SYTO 9 fluorescence intensity. Time to result was compared for ten LAMP primer sets in the presence (1000 genome copies) or absence (0 genome copies) of a target sequence from an RNA virus. Primer sets, namely #1 (SEQ ID NO: 148-SEQ ID NO: 153), #7 (SEQ ID NO: 173-SEQ ID NO: 178), #8 (SEQ ID NO: 174, SEQ ID NO: 176, and SEQ ID NO: 179-SEQ ID NO: 182), and #10 (SEQ ID NO: 187-SEQ ID NO: 192), showed clear differentiation between a sample containing the target sequence and a negative control lacking the target sequence. A decreased time to result is indicative of a sample positive for the target nucleic acid sequence.

FIG. 66 shows fluorescence signal from a DETECTR reaction using a Cas 12 variant (SEQ ID NO: 37) following a five-minute incubation with products from RT-LAMP reactions. LAMP primer sets #1-6 were designed for use with guide RNA #2 (SEQ ID NO: 250), and LAMP primer sets #7-10 were designed for use with guide RNA #1 (SEQ ID NO: 249). Sequences of primers in each primer set are provided in TABLE 14. DETECTR signal was compared for each LAMP primer set in the presence (1000 genome copies) or absence (0 genome copies) of a target sequence using either a guide RNA having a sequence corresponding to SEQ ID NO: 249 (guide RNA#1, top bar graph) or guide RNA having a sequence corresponding to SEQ ID NO: 250 (guide RNA #2, bottom bar graph). Data shows clean differentiation between reactions with the target sequence and no target control reactions when using DETECTR to differentiate between specific and non-specific LAMP amplification. The sequences of the gRNAs used in the DETECTR reaction are provided in TABLE 15.

TABLE 15
DETECTR gRNAs for RT-LAMP Amplification with DETECTR

| SEQ ID NO: | gRNA Name | Sequence |
|---|---|---|
| SEQ ID NO: 249 | gRNA #1 (R1118) | UAAUUUCUACUAAGUGUAGAUCUUAUAA |
| | | AAGAACUAGCCAA |
| SEQ ID NO: 250 | gRNA #2 (R288) | UAAUUUCUACUAAGUGUAGAUACUCAAUU |
| | | UCCUCACUUCUC |

### Example 28

### Detection of Influenza A and B Virus using RT-LAMP and SYTO9

This example describes detection of influenza A and B virus using LAMP and SYTO9. Samples containing either 0, 100, 1000, 10,000, or 100,000 copies of an influenza A virus (IAV) or 0, 100, 1000, 10,000, or 100,000 copies of an influenza B virus (IBV) target nucleic acid sequence were subjected to RT-LAMP amplification using different sets of LAMP primers. Sets of LAMP primers (1, 2, 4, 5, 6, 7, 8, 9, 10, 11, or a negative control) were compared for their ability to specifically amplify the target nucleic acid sequence. Amplification was measured as a time to result using SYTO9. A decreased time to result is indicative of a sample positive for the target nucleic acid sequence.

Each reaction RT-LAMP reaction was performed in the presence of 1× NEB IsoAmp Buffer, 4.5 mM MgSO₄, 6.4 U/μL Bst 2.0 (NEB), 0.75 μL Warmstart RTx reverse transcriptase, 1 μL 10×0 primer mix, and 0.2 μL SYTO9 per 10 μL reaction in nuclease free water.

FIG. 67 shows detection of sequences from influenza A virus (IAV) using SYTO 9 (a DNA binding dye) following RT-LAMP amplification with LAMP primer sets 1, 2, 4, 5, 6, 7, 8, 9, 10, 11, or a negative control. Ten reactions were performed per primer set and reactions were performed in duplicate. Individual plots depict fluorescence intensity over time during the LAMP amplification reaction. Fluorescence from SYTO 9 was measured over time as a function of an amount of target sequence present in the reaction. Plots in rows show amplification in the presence of, from top to bottom, 0, 100, 1000, 10,000, or 100,000 copies of the target nucleic acid. Plots in columns show amplification using, from left to right, primer sets 1, 2, IBV, 4, 5, 6, 7, 8, 9, 10, and 11. Primer set 1 (SEQ ID NO: 193-SEQ ID NO: 198) shows a flat negative control curve, indicating suitability for use in LAMP amplification reactions. Primer set 2 (SEQ ID NO: 199-SEQ ID NO: 204) is well-suited for use in amplifying a target nucleic acid using LAMP. Primer set 8 (SEQ ID NO: 220-SEQ ID NO: 225) and primer set 10 (SEQ ID NO: 221, SEQ ID NO: 223-SEQ ID NO: 225, and SEQ ID NO: 229-SEQ ID NO: 230) also work well in amplifying a target nucleic acid using LAMP. Primer set 8 produces a lower negative control amplification signal than primer set 10.

FIG. 69 shows the time to amplification of an IAV target sequence following LAMP amplification with different primer sets as determined from the SYTO 9 fluorescence traces shown in FIG. 67. Time to result was determined as the time to reach half maximum SYTO 9 fluorescence intensity. Amplification was detected using SYTO9 in the presence of increasing concentrations of target sequence (0, 100, 1000, 10,000, or 100,000 genome copies of the target sequence per reaction). The assay was capable of distinguishing between negative control reactions (no target sequence) and reactions containing 100,000 genome copies of the target sequence for all primer sets. The sequences of the LAMP primers in each primer set are provided in TABLE 16.

TABLE 16
Primers for Amplification and Detection of IAV and IBV Virus using RT-LAMP

| SEQ ID NO: | Primer Name | Primer Set | Sequence |
|---|---|---|---|
| SEQ ID NO: 193 | IAV-MP-F3 | #1 | GACTTGAAGATGTCTTTGC |
| SEQ ID NO: 194 | IAV-MP B3 | #1 | TGTTGTTTGGGTCCCCATT |
| SEQ ID NO: 195 | IAV-MP-FIP | #1 | TTAGTCAGAGGTGACAGGATTGC |
| | | | AGATCTTGAGGCTCTC |
| SEQ ID NO: 196 | IAV-MP-BIP | #1 | TTGTGTTCACGCTCACCGTGTTTG |
| | | | GACAAAGCGTCTACG |
| SEQ ID NO: 197 | IAV-MP FL | #1 | GTCTTGTCTTTAGCCA |
| SEQ ID NO: 198 | IAV-MP BL | #1 | CAGTGAGCGAGGACTG |
| SEQ ID NO: 199 | IAV F3 v2 | #2 | ACCGAGGTCGAAACGT |
| SEQ ID NO: 200 | IAV B3 v2 | #2 | GGTCCCCATTCCCATTG |
| SEQ ID NO: 201 | IAV FIP v2 | #2 | CAAAGACATCTTCAAGTCTCTGCG |
| | | | TTTTTTCTCTCTATCGTCCCGTCA |
| SEQ ID NO: 202 | IAV BIP v2 | #2 | AATGGCTAAAGACAAGACCAATC |
| | | | CTTTTTTGTCTACGCTGCAGTCC |
| SEQ ID NO: 203 | IAV LF v2 | #2 | CGATCTCGGCTTTGAGGG |
| SEQ ID NO: 204 | IAV LB v2 | #2 | TCACCGTGCCCAGTGAG |
| SEQ ID NO: 205 | IAV F3 v3 | #3 | CGAAAGCAGGTAGATATTGAAAG |
| SEQ ID NO: 206 | IAV B3 v3 | #3 | TCTACGCTGCAGTCCTC |
| SEQ ID NO: 207 | IAV FIP v3 | #3 | TCAAGTCTCTGCGCGATCTCTTTT |
| | | | TTGAGTCTTCTAACCGAGGT |
| SEQ ID NO: 208 | IAV BIP v3 | #3 | AGATGTCTTTGCAGGGAAAAACA |
| | | | CTTTTTTCACAAATCCTAAAATCC |
| | | | CCTTAG |
| SEQ ID NO: 209 | IAV LF v3 | #3 | GACGATAGAGAGAACGTACGTTT |
| | | | C |
| SEQ ID NO: 210 | IAV LB v3 | #3 | AAGACCAATCCTGTCACCTCT |
| SEQ ID NO: 211 | IAV-set4-F3 | #4 | GCGAAAGCAGGTAGATATTGA |
| SEQ ID NO: 212 | IAV-set4-B3 | #4 | CATTCCCATTGAGGGCATT |
| SEQ ID NO: 213 | IAV-set4-FIP | #4 | CTTCAAGTCTCTGCGCGATCTATG |
| | | | AGTCTTCTAACCGAGGT |
| SEQ ID NO: 214 | IAV-set4-BIP | #4 | TTGAGGCTCTCATGGAATGGCAG |
| | | | CGTGAACACAAATCCTAA |
| SEQ ID NO: 215 | IAV-set4-LF | #4 | TGACGGGACGATAGAGAGAA |
| SEQ ID NO: 216 | IAV-set4-LB | #4 | ACAAGACCAATCCTGTCACC |
| SEQ ID NO: 211 | IAV-set5-F3 | #5 | GCGAAAGCAGGTAGATATTGA |
| SEQ ID NO: 212 | IAV-set5-B3 | #5 | CATTCCCATTGAGGGCATT |
| SEQ ID NO: 217 | IAV-set5-FIP | #5 | TTCAAGTCTCTGCGCGATCTCATG |
| | | | AGTCTTCTAACCGAGGT |
| SEQ ID NO: 214 | IAV-set5-BIP | #5 | TTGAGGCTCTCATGGAATGGCAG |
| | | | CGTGAACACAAATCCTAA |
| SEQ ID NO: 215 | IAV-set5-LF | #5 | TGACGGGACGATAGAGAGAA |
| SEQ ID NO: 216 | IAV-set5-LB | #5 | ACAAGACCAATCCTGTCACC |
| SEQ ID NO: 211 | IAV-set6-F3 | #6 | GCGAAAGCAGGTAGATATTGA |
| SEQ ID NO: 218 | IAV-set6-B3 | #6 | TTGGACAAAGCGTCTACG |
| SEQ ID NO: 213 | IAV-set6-FIP | #6 | CTTCAAGTCTCTGCGCGATCTATG |
| | | | AGTCTTCTAACCGAGGT |
| SEQ ID NO: 214 | IAV-set6-BIP | #6 | TTGAGGCTCTCATGGAATGGCAG |
| | | | CGTGAACACAAATCCTAA |
| SEQ ID NO: 215 | IAV-set6-LF | #6 | TGACGGGACGATAGAGAGAA |
| SEQ ID NO: 216 | IAV-set6-LB | #6 | ACAAGACCAATCCTGTCACC |
| SEQ ID NO: 211 | IAV-set7-F3 | #7 | GCGAAAGCAGGTAGATATTGA |
| SEQ ID NO: 212 | IAV-set7-B3 | #7 | CATTCCCATTGAGGGCATT |
| SEQ ID NO: 219 | IAV-set7-FIP | #7 | AAGTCTCTGCGCGATCTCGATGA |
| | | | GTCTTCTAACCGAGGT |
| SEQ ID NO: 214 | IAV-set7-BIP | #7 | TTGAGGCTCTCATGGAATGGCAG |
| | | | CGTGAACACAAATCCTAA |
| SEQ ID NO: 215 | IAV-set7-LF | #7 | TGACGGGACGATAGAGAGAA |
| SEQ ID NO: 216 | IAV-set7-LB | #7 | ACAAGACCAATCCTGTCACC |
| SEQ ID NO: 220 | IAV-set8-F3 | #8 | TCTTCTAACCGAGGTCGAA |
| SEQ ID NO: 221 | IAV-set8-B3 | #8 | CTGCTCTGTCCATGTTGTT |
| SEQ ID NO: 222 | IAV-set8-FIP | #8 | TCAGAGGTGACAGGATTGGTCTG |
| | | | AAGATGTCTTTGCAGGGAA |
| SEQ ID NO: 223 | IAV-set8-BIP | #8 | TTGTGTTCACGCTCACCGTCATTC |
| | | | CCATTGAGGGCATT |
| SEQ ID NO: 224 | IAV-set8-LF | #8 | ATTCCATGAGAGCCTCAAGATC |
| SEQ ID NO: 225 | IAV-set8-LB | #8 | GAGGACTGCAGCGTAGAC |
| SEQ ID NO: 226 | IAV-set9-F3 | #9 | TTCTCTCTATCGTCCCGTC |
| SEQ ID NO: 221 | IAV-set9-B3 | #9 | CTGCTCTGTCCATGTTGTT |
| SEQ ID NO: 227 | IAV-set9-FIP | #9 | CCCTTAGTCAGAGGTGACAGGAA |
| | | | CACAGATCTTGAGGCTCT |
| SEQ ID NO: 223 | IAV-set9-BIP | #9 | TTGTGTTCACGCTCACCGTCATTC |
| | | | CCATTGAGGGCATT |
| SEQ ID NO: 228 | IAV-set9-LF | #9 | GGTCTTGTCTTTAGCCATTCCA |
| SEQ ID NO: 225 | IAV-set9-LB | #9 | GAGGACTGCAGCGTAGAC |
| SEQ ID NO: 229 | IAV-set10-F3 | #10 | GTCTTCTAACCGAGGTCGA |
| SEQ ID NO: 221 | IAV-set10-B3 | #10 | CTGCTCTGTCCATGTTGTT |
| SEQ ID NO: 230 | IAV-set10-FIP | #10 | GAGGTGACAGGATTGGTCTTGTT |
| | | | GAAGATGTCTTTGCAGGG |
| SEQ ID NO: 223 | IAV-set10-BIP | #10 | TTGTGTTCACGCTCACCGTCATTC |
| | | | CCATTGAGGGCATT |
| SEQ ID NO: 224 | IAV-set10-LF | #10 | ATTCCATGAGAGCCTCAAGATC |
| SEQ ID NO: 225 | IAV-set10-LB | #10 | GAGGACTGCAGCGTAGAC |
| SEQ ID NO: 231 | IAV-set11-F3 | #11 | AAGAAGACAAGAGATATGGC |
| SEQ ID NO: 232 | IAV-set11-B3 | #11 | CAATTCGACACTAATTGATGGC |
| SEQ ID NO: 233 | IAV-set11-FIP | #11 | GTCTCCTTGCCCAATTAGCAAGCA |
| | | | TCAATGAACTGAGCA |
| SEQ ID NO: 234 | IAV-set11-BIP | #11 | GTGGTGTTGGTAATGAAACGAAG |
| | | | CTGTCTGGCTGTCAGTA |
| SEQ ID NO: 235 | IAV-set11-LF | #11 | ACATTAGCCTTCTCTCCTTT |
| SEQ ID NO: 236 | IAV-set11-LB | #11 | AACGGGACTCTAGCATACT |
| SEQ ID NO: 237 | M605 F3 IBV | IBV | AGGGACATGAACAACAAAGA |
| | LAMP | | |
| SEQ ID NO: 238 | M606 B3 IBV | IBV | CAAGTTTAGCAACAAGCCT |
| | LAMP | | |
| SEQ ID NO: 239 | M607 FIP IBV | IBV | TCAGGGACAATACATTACGCATA |
| | LAMP | | TCGATAAAGGAGGAAGTAAACAC |
| | | | TCA |
| SEQ ID NO: 240 | M608 BIP IBV | IBV | TAAACGGAACATTCCTCAAACAC |
| | LAMP | | CACTCTGGTCATATGCATTC |
| SEQ ID NO: 241 | M609 LF IBV | IBV | TCAAACGGAACTTCCCTTCTTTC |
| | LAMP | | |
| SEQ ID NO: 242 | M610 LB IBV | IBV | GGATACAAGTCCTTATCAACTCTG |
| | LAMP | | C |

FIG. 68 shows the time to amplification of an influenza B virus (IBV) target sequence following RT-LAMP amplification. Amplification was detected using SYTO9 in the presence of increasing concentrations of the target nucleic acid sequence (0, 100, 1000, 10,000, or 100,000 genome copies of the target sequence per reaction). RT-LAMP amplification was performed using primer set #8 (SEQ ID NO: 220-SEQ ID NO: 225), provided in TABLE 16.

### Example 29

### Detection of Influenza A Virus using LAMP and DETECTR

This example describes detection of influenza A virus using LAMP and DETECTR. Samples containing an influenza A virus (IAV) target nucleic acid sequence or lacking the IAV target nucleic acid sequence were subjected to RT-LAMP amplification using different sets of LAMP primers. Sets of LAMP primers were compared for their ability to specifically amplify the target nucleic acid sequence. Presence or absence of the target nucleic acid in the sample was subsequently measured using DETECTR. DETECTR signal, measured by an increase in fluorescent signal upon activation of a programmable nuclease, was observed over time. An increase in fluorescence indicates the presence of the target nucleic acid sequence.

Each RT-LAMP reaction was performed in the presence of 1× NEB IsoAmp Buffer, 4.5 mM MgSO₄, 1.4 mM dNTPs (NEB), 6.4 U/μL Bst 2.0 (NEB), 1.5 μL Warmstart RTx, and 2 μL 10× primer mix per 20 μL reaction in nuclease-free water. Each DETECTR reaction was performed in the presence of lx Processing Buffer, 250 nM crRNA, and 200 nM Sr-WT LbCas12a (SEQ ID NO: 27) programmable nuclease in nuclease-free water.

FIG. 70 shows detection of target nucleic acid sequences from influenza A virus (IAV) using DETECTR following RT-LAMP amplification with LAMP primer sets 1, 2, 4, 5, 6, 7, 8, 9, 10, or a negative control. RT-LAMP amplification was performed using the primer sets provided in TABLE 16. Ten reactions were performed per primer set. DETECTR was performed with different gRNAs. The sequences of the gRNAs used in the DETECTR reaction are provided in TABLE 17. DETECTR signal was measured as a function of an amount of target sequence present in the reaction. Individual plots depict fluorescence intensity over time during DETECTR reaction following LAMP amplification. Individual traces on each plot show amplification followed by DETECTR with a guide RNA corresponding to SEQ ID NO: 251 (R283 gRNA, blue), a guide RNA corresponding to SEQ ID NO: 252 (R781 gRNA, red), a guide RNA corresponding to SEQ ID NO: 253 (R782 gRNA, green), or a guide RNA corresponding to SEQ ID NO: 254 (IBV gRNA, purple). Plots in rows show DETECTR following LAMP amplification in the presence of, from top to bottom, 0, 100, 1000, 10,000, or 100,000 copies of the target nucleic acid. Plots in columns show DETECTR following LAMP amplification using, from left to right, primer sets 1, 2, 4, 5, 6, 7, 8, 9, 10, 11, or IBV. Using primer set 1 resulted in robust amplification of the target nucleic acid by RT-LAMP. Primer set 2 was also found to be well-suited for use in combined methods of amplifying a target nucleic acid sequence by RT-LAMP and detecting the target nucleic acid sequence by DETECTR. Primer set 8 (SEQ ID NO: 220-SEQ ID NO: 225) and primer set 10 (SEQ ID NO: 221, SEQ ID NO: 223-SEQ ID NO: 225, and SEQ ID NO: 229-SEQ ID NO: 230) were well suited for use in combined RT-LAMP and DETECTR reactions when detected using the guide RNA corresponding to SEQ ID NO: 253 analyzed with R782, as indicated by robust amplification and detection of the target nucleic acid without nonspecific amplification or detection in the absence of the target nucleic acid. Target nucleic acid sequences from IBV were also detected by DETECTR after RT-LAMP amplification of the target.

TABLE 17
DETECTR gRNAs for RT-LAMP Amplification with DETECTR of IAV or IBV

| SEQ ID NO: | gRNA Name | Sequence |
|---|---|---|
| SEQ ID NO: 251 | R283 | UAAUUUCUACUAAGUGUAGAUUGUUCACG |
| | | CUCACCGUGCCC |
| SEQ ID NO: 252 | R781 | UAAUUUCUACUAAGUGUAGAUGCCAUUCC |
| | | AUGAGAGCCUCA |
| SEQ ID NO: 253 | R782 | UAAUUUCUACUAAGUGUAGAUGACAAAGC |
| | | GUCUACGCUGCA |
| SEQ ID NO: 254 | IBV (R778) | UAAUUUCUACUAAGUGUAGAUCUAACACU |
| | | CUCAGGGACAAU |

### Example 30

### Detection of a SNP using LAMP and DETECTR

This example describes detection of a SNP using LAMP and DETECTR. Strategies for designing primers for use in combined LAMP and DETECTR reactions to detect SNPs were tested and evaluated for multiple target SNPs. From these experiments, a set of design guidelines was determined to facilitate combined LAMP and DETECTR reactions for DNA nucleic acid targets or RT-LAMP and DETECTR reactions for RNA nucleic acid targets.

FIG. 71 shows a scheme for designing primers for LAMP amplification of a target nucleic acid sequence and detection of a single nucleotide polymorphism (SNP) in the target nucleic acid sequence. In an exemplary arrangement, the SNP of the target nucleic acid is positioned between the F1c region and the B1 region.

FIG. 72 shows schematics of exemplary arrangements of LAMP primers, guide RNA sequences, protospacer-adjacent motif (PAM) or protospacer flanking site (PFS), and target nucleic acids with a SNP for methods of LAMP amplification of a target nucleic acid and detection of the target nucleic acid using DETECTR.

FIG. 72A shows a schematic of an exemplary arrangement of the guide RNA with respect to various regions of the nucleic acid sequence that correspond to or anneal LAMP primers. In this arrangement, the PAM or PFS of the target nucleic acid is positioned between an F1c region and a B1 region. The entirety of the guide RNA sequence may be between the F1c region and the B1c region. The SNP is shown as positioned within a sequence of the target nucleic acid that hybridizes to the guide RNA.

FIG. 72B shows a schematic of an exemplary arrangement of the guide RNA sequence with respect to various regions of the nucleic acid sequence that correspond to or anneal LAMP primers. In this arrangement, the PAM or PFS of the target nucleic acid is positioned between an F1c region and a B1 region and the target nucleic acid comprises a sequence between an F1c region and a B1 region that is reverse complementary to at least 60% of a guide nucleic acid. In this example, the guide RNA is not reverse complementary to the forward inner primer or the backward inner primer. The SNP is shown as positioned within a sequence of the target nucleic acid that hybridizes to the guide RNA.

FIG. 72C shows a schematic of an exemplary arrangement of the guide RNA sequence with respect to various regions of the nucleic acid sequence that correspond to or anneal LAMP primers. In this arrangement, the PAM or PFS of the target nucleic acid is positioned between the F1c region and the B1 region and the entirety of the guide RNA sequence is between the F1c region and the B1 region. The SNP is shown as positioned within a sequence of the target nucleic acid that hybridizes to the guide RNA.

FIG. 73 shows an exemplary sequence of a nucleic acid comprising two PAM sites and a HERC2 SNP. The SNP is positioned at position 9 relative to a first PAM site or position 14 relative to a second PAM site.

FIG. 74 shows results from DETECTR reactions to detect a HERC2 SNP at position 9 relative to a first PAM site or position 14 relative to a second PAM site following LAMP amplification. The SNP position is indicated by a triangle. Fluorescence signal, indicative of detection of the target sequence, was measured over time in the presence of a target sequence comprising either a G SNP allele or an A SNP allele in HERC2. The target nucleic acid comprising the SNP was amplified using the primers presented in TABLE 18.

TABLE 18
LAMP Primers for Amplification and Detection of a HERC2 SNP

| SEQ ID NO: | Primer Name | Primer Set | Sequence |
|---|---|---|---|
| SEQ ID NO: 243 | M948 F3 | HERC2 | CTTGTAATCAACATCAGGGTAA |
| | HERC2 set3 | | |
| SEQ ID NO: 244 | M949 B3 | HERC2 | AGAAACGACAAGTAGACCATT |
| | HERC2 set3 | | |
| SEQ ID NO: 245 | M950 FIP | HERC2 | CGCCTCTTGGATCAGACACATGTG |
| | HERC2 set3 | | TTAATACAAAGGTACAGGA |
| SEQ ID NO: 246 | M951 BIP | HERC2 | CACGCTATCATCATCAGGGGCTG |
| | HERC2 set3 | | CTTCAAGTGTATATAAACTCAC |
| SEQ ID NO: 247 | M952 LF | HERC2 | GAGAGCCATGAAGAACAAATTCT |
| | HERC2 set3 | | |
| SEQ ID NO: 248 | M953 LB | HERC2 | CGAGGCTTCTCTTTGTTTTTAAT |
| | HERC2 set3 | | |

The target sequence was detected using a guide RNA (crRNA only) to detect either the A allele with the first PAM site (SNP Position 9, “A SNP”), the G allele with the first PAM site (SNP Position 9, “G SNP”), the A allele with the second PAM site (SNP Position 14, “A SNP”)or the G allele with the second PAM site (SNP Position 14, “G SNP”). Four guide RNAs designed for each condition were used. The guide RNAs used for the detection of the two SNP alleles relative to the two PAM sites are presented in TABLE 19. The guide RNA corresponding to SEQ ID NO: 255 was designed to detect the A allele at position 9, the guide RNA corresponding to SEQ ID NO: 256 was designed to detect the G allele at position 9, the guide RNA corresponding to SEQ ID NO: 257 was designed to detect the A allele at position 14, and the guide RNA corresponding to SEQ ID NO: 258 was designed to detect the G allele at position 14. A high fluorescence signal was detected for the G allele in the presence of the position 9 G SNP guide RNA (SEQ ID NO: 256, top left) and the A allele in the presence of the position 9 A SNP guide RNA (SEQ ID NO: 255, bottom right). Minimal fluorescence signal was detected for the G allele in the presence of the position 9 A SNP guide RNA (SEQ ID NO: 255, top right) and the position 9 A allele in the presence of the G SNP guide RNA (SEQ ID NO: 256, bottom left). This indicates that the position 9 G SNP and position 9 A SNP guide RNAs show specificity for the G allele and A allele, respectively. The position 14 A SNP guide RNA (SEQ ID NO: 257) and the position 14 G SNP guide RNA (SEQ ID NO: 258) detected both alleles, as shown by high fluorescence signal when detecting the SNP with the position 14 A SNP or G SNP guide RNAs, independent of the target sequence present.

FIG. 75 shows a heatmap of fluorescence from a DETECTR reaction following LAMP amplification of the target nucleic acid sequence. The DETECTR reaction differentiated between two HERC2 SNP alleles, using guide RNAs (crRNA only) specific for the A allele (SEQ ID NO: 255) or the G allele (SEQ ID NO: 256). Positive detection is indicated by a high fluorescence value in the DETECTR reaction. Guide RNA corresponding to SEQ ID NO: 255 was specific for A allele, as indicated by (i) a high fluorescence signal in the A SNP positive control, the HeLa sample, and Sample 2, and (ii) low fluorescence signal in the G SNP positive control, the negative control, and Sample 1. Guide RNA corresponding to SEQ ID NO: 256 was specific for G allele, as indicated by (i) a high fluorescence signal in the G SNP positive control, the HeLa sample, and Sample 1, and (ii) low fluorescence signal in the A SNP positive control, the negative control, and Sample 2. Sample 1 was homozygous for the G allele and Sample 2 was homozygous for the A allele.

TABLE 19
DETECTR Guide RNAs for Amplification and Detection of a HERC2 SNP

| SEQ ID NO: | gRNA Name | Sequence |
|---|---|---|
| SEQ ID NO: 255 | A SNP Position 9 | UAAUUUCUACUAAGUGUAGAUAGCAUUA |
| | (R570) | AAUGUCAAGUUCU |
| SEQ ID NO: 256 | G SNP Position 9 | UAAUUUCUACUAAGUGUAGAUAGCAUUA |
| | (R571) | AGUGUCAAGUUCU |
| SEQ ID NO: 257 | A SNP Position 14 | UAAUUUCUACUAAGUGUAGAUAUUUGAG |
| | (R1138) | CAUUAAAUGUCAA |
| SEQ ID NO: 258 | G SNP Position 14 | UAAUUUCUACUAAGUGUAGAUAUUUGAG |
| | (R1139) | CAUUAAGUGUCAA |

FIG. 76 shows combined LAMP amplification of a target nucleic acid by LAMP and detection of the target nucleic acid by DETECTR. Detection was carried out visually with DETECTR by illuminating the samples with a red LED. Each reaction contained a target nucleic acid sequence comprising a SNP allele for either a blue eye phenotype (“Blue Eye”) or a brown eye phenotype (“Brown Eye”). Samples “Brown *” and “Blue *” were an A allele positive control and a G allele positive control, respectively. A position 9 guide RNA for either the brown eye phenotype (SEQ ID NO: 255, “Br”) or the blue eye phenotype (SEQ ID NO: 256, “Bl”) was used for each LAMP DETECTR reaction. The presence of either the blue eye allele or the brown eye allele was visually detected by eye, as shown by an increase in fluorescence in each tube containing a target nucleic acid sequence and a corresponding guide RNA. The guide RNA for the brown eye allele (SEQ ID NO: 255) was specific for the A allele, as shown by a high fluorescence signal (brighter tubes) in tubes containing the brown eye guide RNA and either the brown eye target nucleic acid or the A SNP positive control, and low fluorescence signal (darker tubes) in tubes containing the brown eye guide RNA and either the blue eye target nucleic acid or the G SNP positive control. The guide RNA for the blue eye allele (SEQ ID NO: 256) was specific for the G allele, as shown by a high fluorescence signal (brighter tubes) in tubes containing the blue eye guide RNA and either the blue eye target nucleic acid or the G SNP positive control, and low fluorescence signal (darker tubes) in tubes containing the blue eye guide RNA and either the brown eye target nucleic acid or the A SNP positive control.

### Example 31

### RT-LAMP DETECTR Reactions for Detection of Coronavirus

This example describes RT-LAMP DETECTR reactions for the detection of coronavirus. SARS-CoV-2 target sequences were designed using all available genomes available from GISAID. Briefly, viral genomes were aligned using Clustal Omega. Next, LbCas12a target sites on the SARS-CoV-2 genome were filtered against SARS-CoV, two bat-SARS-like-CoV genomes and common human coronavirus genomes. Compatible target sites were finally compared to those used in current protocols from the CDC and WHO. LAMP primers for SARS-CoV-2 were designed against regions of the N-gene and E-gene using PrimerExplorer v5 (https://primerexplorer.jp/e/). FIG. 114A shows a sequence alignment of the target sites targeted by the N-gene gRNA for three coronavirus strains. The N gene gRNA #1 is compatible with the CDC-N2 amplicon, the N gene gRNA #2 is compatible with WHO N-Sarbeco amplicon. FIG. 114B shows a sequence alignment of the target sites targeted by the E-gene gRNA for three coronavirus strains. The two E gene gRNAs tested (E gene gRNA #1 and E gene gRNA #2) are compatible with the WHO E-Sarbeco amplicon. RNase P POP7 primers were originally published by Curtis, et al. (2018) and a compatible gRNA was designed to function with these primer sets.

Target RNAs were generated from synthetic gene fragments of the viral genes of interest. First a PCR step was performed on the synthetic gene fragment with a forward primer that contained a T7 promoter. Next, the PCR product was used as the template for an in-vitro transcription (IVT) reaction at 37° C. for 2 hours. The IVT reaction was then treated with TURBO DNase (Thermo) for 30 minutes at 37° C., followed by a heat-denaturation step at 75° C. for 15 minutes. RNA was purified using RNA Clean and Concentrator 5 columns (Zymo Research). RNA was quantified by Nanodrop and Qubit and diluted in nuclease-free water to working concentrations.

DETECTR assays were performed using RT-LAMP for pre-amplification of viral or control RNA targets and LbCas12a for the trans-cleavage assay. RT-LAMP was prepared with a MgSO₄ concentration of 6.5 mM and a final volume of 10 μL. LAMP primers were added at a final concentration of 0.2 μM for F3 and B3, 1.6 μM for FIP and BIP, and 0.8 μM for LF and LB. Reactions were performed independently for N-gene, E-gene, and RNase P using 2 μL of input RNA at 62° C. for 20 minutes.

For LbCas12a (SEQ ID NO: 27) trans-cleavage, 50 nM LbCas12a (available from NEB) was pre-incubated with 62.5 nM gRNA in 1X NEBuffer 2.1 for 30 minutes at 37° C. After formation of the RNA-protein complex, the lateral flow cleavage reporter (/56-FAM/TTATTATT/3Bio/ (SEQ ID NO: 9), IDT) was added to the reaction at a final concentration of 500 nM. RNA-protein complexes were used immediately or stored at 4° C. for up to 24 hours before use.

After completion of the pre-amplification step, 2 μL of amplicon was combined with 18 μL of LbCas12a-gRNA complex and 80 μL of 1× NEBuffer 2.1. The 100 μL LbCas12a trans-cleavage assay was allowed to proceed for 10 minutes at 37° C.

A lateral flow strip (Milenia HybriDetect 1, TwistDx) was then added to the reaction tube and a result was visualized after approximately 2-3 minutes. A single band, close to the sample application pad indicated a negative result, whereas a single band close to the top of the strip or two bands indicated a positive result.

The patient optimized DETECTR assays were performed using RT-LAMP method as described above with the following modifications: A DNA binding dye, SYTO9 (Thermo Fisher Scientific), was included in the reaction to monitor the amplification reaction and the incubation time was extended to 30 minutes to capture data from lower titre samples.

The fluorescence based patient optimized LbCas12a trans-cleavage assays were performed as described above with modifications; 40 nM LbCas12a was pre-incubated with 40 nM gRNA, after which 100 nM of a fluorescent reporter molecule compatible with detection in the presence of the SYTO9 dye (/5Alex594N/TTATTATT/3IAbRQSp/ (SEQ ID NO: 9)) was added to the complex. 2 μL of amplicon was combined with 18 μL of LbCas12a-gRNA complex in a black 384-well assay plate and monitored for fluorescence using a Tecan plate reader.

### Example 32

### Screening of Primer Sets for Amplification of a SARS-CoV-2 Target Site

This example describes the screening of primer sets for amplification of a SARS-CoV-2 target site. A region of the coronavirus RNA genome corresponding to the viral N-gene was amplified using different LAMP primer sets (sett through set 11). Samples containing either 1.5 pM, 5 fM, or 0 fM SARS-CoV-2 RNA were amplified with each primer set. SARS-CoV-2 RNA in each sample was reverse transcribed using a warmstart reverse transcriptase (“Warmstart RTx”) and LAMP amplified using a Bst 2.0 DNA polymerase. The assay was performed at 60 degrees C. for 60 minutes. FIG. 77 illustrates schematically the steps of preparing and detecting a sample using a RT-LAMP and Cas12 DETECTR reactions. FIG. 97 shows technical specifications and assay conditions for detection of coronavirus using reverse transcription and loop-mediated isothermal amplification (RT-LAMP) and Cas12 detection.

A DETECTR assay was performed on each amplified sample, and the time to result was determined. Sequences were detected using a gRNA sequence corresponding to R1763 directed to the N-gene of SARS-CoV-2 and a Cas12 programmable nuclease corresponding to LbCas12a. The DETECTR assay was sensitive for the amplified SARS-CoV-2 target sequence for all tested primer sets. Sequences of the gRNAs used in this example are provided in TABLE 20. FIG. 78 shows the DETECTR assay results of the SARS-CoV-2 N-gene amplified with different primer sets (“2019-nCoV-setl” through “2019-nCoV-set12”) and detected using LbCas12a and a gRNA directed to the N-gene of SARS-CoV-2 (“R1763,” SEQ ID NO: 323). A lower time to result is indicative of a positive result. For all primer sets, the time to result was lower for samples with more of the target sequence, indicating that the assay was sensitive for the target sequence. FIG. 79 shows the individual traces of the DETECTR reactions plotted in FIG. 78 for the 0 fM and 5 fM samples. In each plot, the 0 fM trace is not visible above the baseline, indicating that there little to no non-specific detection. The best performing primer set for R1763 (SEQ ID NO: 323) was SARS-CoV-2-N-setl. Time to detect was less than 10 minutes at the tested concentration.

In a second assay, primer sets directed to the E-gene of Sarbeco (detected with gRNAs R1764 and R1765) and the N-gene of Sarbeco (detected with R1767). FIG. 80 shows the time to result of a DETECTR reaction on samples containing either the N-gene, the E-gene, or no target (“NTC”). Samples were amplified using primer sets directed to the E-gene of SARS-CoV-2 (“2019-nCoV-E-set13” through “2019-nCoV-E-set20”) or to the N-gene of SARS-CoV-2 (“2019-nCoV-N-set21” through “2019-nCoV-N-set24”). Target site sequences are provided in TABLE 21. The best performing primer set was SARS-CoV-2-E-set14. The presence of the SARS-CoV-2 N-gene was detected using the R1767 N-gene gRNA (SEQ ID NO: 327) and the presence of the SARS-CoV-2 E-gene was detected using either the R1764 E-gene gRNA (SEQ ID NO: 324) or the R1765 E-gene gRNA (SEQ ID NO: 325).

A control primer set for amplifying RNase P was also tested. FIG. 83 shows the amplification of RNase P using a POP7 sample primer set. Samples were amplified using LAMP. DETECTR reactions were performed using a gRNA directed to RNase P (“R779,” SEQ ID NO: 330) and a Cas12 variant (SEQ ID NO: 37). Samples contained either HeLa total RNA or HeLa genomic DNA. A series of line graphs is shown in the bottom half of FIG. 83. At the top of the bottom half of FIG. 83 are graphs of DETECTR reactions with HeLa total RNA as the target, which from left to right show decreasing concentrations of the target as follows: 20 ng/μl, 4 ng/μl, 0.8 ng/μl, 0.16 ng/μl, 0.032 ng/μl, 0.0064 ng/μl, 0.00128 ng/μl, and 0 ng/μl. The x-axis shows time in minutes ranging from 0 to 30 in increments of 10. The y-axis shows raw fluorescence in arbitrary units (AU) ranging from 0 to 50000 in increments of 10000. At the bottom of the bottom half of FIG. 83 are graphs of DETECTR reactions with HeLa genomic DNA as the target, which from left to right show decreasing concentrations of the target as follow: 20 ng/μl, 4 ng/μl, 0.8 ng/μl, 0.16 ng/μl, 0.032 ng/μl, 0.0064 ng/μl, 0.00128 ng/μl, and 0 ng/μl. The x-axis shows time in minutes ranging from 0 to 30 in increments of 10. The y-axis shows raw fluorescence in arbitrary units (AU) ranging from 0 to 50000 in increments of 10000.

### Example 33

### Specificity of Detection of a SARS-CoV-2 Target Nucleic Acid

This example describes the specificity of detection of a SARS-CoV-2 target nucleic acid. A sample containing target RNA corresponding to SARS-CoV-2 was amplified as using primer set 1 as described in EXAMPLE 2. gRNAs were screened for compatibility with different primer sets designed to amplify either the N-gene or the E-gene of SARS-CoV-2. FIG. 98 shows the results of a DETECTR assay evaluating multiple gRNAs for detecting SARS-CoV-2 using LbCas12a. Target nucleic acid sequences were amplified using primer sets to amplify the SARS-CoV-2 E-gene (“2019-nCoV-E-set13” through “2019-nCoV-E-set20” or the SARS-CoV-2 N-gene (“2019-nCoV-N-set21” through “2019-nCoV-N-set24”). The gRNA corresponding to SEQ ID NO: 324 (“R1764-E-Sarbeco-1) and the gRNA corresponding to SEQ ID NO: 325 (“R1765—E-Sarbeco-2”) were able to detect target sequences amplified using LAMP primer sets directed to the E-gene of SARS-CoV-2. The gRNA corresponding to SEQ ID NO: 327 (“R1767—N-Sarbeco”) was ample to detect target sequences amplified using most LAMP primer sets directed to the N-gene of SARS-CoV-2. FIG. 98 shows a series of line graphs showing DETECTR reactions, which from left to right are 2019-nCoV-E-set13, 2019-nCoV-E-set14, 2019-nCoV-E-set15, 2019-nCoV-E-set16, 2019-nCoV-E-set17, 2019-nCoV-E-set18, 2019-nCoV-E-set19, 2019-nCoV-E-set20, 2019-nCoV-E-set21, 2019-nCoV-E-set22, 2019-nCoV-E-set23, and 2019-nCoV-E-set24. In the top row of graphs, the second, third, and fourth graph from the right show that R1767 exhibits the highest signal most quickly. In the middle row of graphs, the first 8 graphs from the left show that R1764 and R1765 exhibit the highest signal most quickly. In the bottom row of graphs, the sixth graph from the left shows that R1764 and R1765 exhibit the highest signal most quickly. The x-axis on all the graphs shows time in minutes ranging from 0 to 75 in increments of 25 and the y-axis on all the graphs shows raw fluorescence in arbitrary units (AU) ranging from 0 to 60000 in increments in 20000.

Samples containing either 5 fM or 0 fM SARS-CoV-2 RNA were detected using a DETECTR assay. Samples were detected using LbCas12a and either a gRNA R1763 directed to the N-gene of SARS-CoV-2 or a gRNA R1766 directed to the N-gene of SARS-CoV. Sequences of the gRNAs used in this example are provided in TABLE 20. FIG. 81 shows the DETECTR assay results of the SARS-CoV-2 N-gene amplified with primer set 1 (“2019-nCoV-sett”) and detected using LbCas12a (SEQ ID NO: 27) and either a gRNA directed to the N-gene of SARS-CoV-2 (“R1763—CDC-N2-Wuhan,” SEQ ID NO: 323) or a gRNA directed to the N-gene of SARS-CoV (“R1766—CDC-N2-SARS,” SEQ ID NO: 326).

FIG. 87A schematically illustrates the sequence of the CDC-N2 target site used for detecting the N-2 gene of SARS-CoV-2 in this assay. Target site sequences are provided in TABLE 21.

TABLE 20
Exemplary gRNA Sequences for Detection of Coronaviruses

| SEQ ID NO: | gRNA | Target | Sequence |
|---|---|---|---|
| SEQ ID NO: 323 | R1763 | CDC-N2-Wuhan | UAAUUUCUACUAAGUGUAGAUCC |
| | | | CCCAGCGCUUCAGCGUUC |
| SEQ ID NO: 324 | R1764 | E-Sarbeco-1 | UAAUUUCUACUAAGUGUAGAUUU |
| | | | GCUUUCGUGGUAUUCUUG |
| SEQ ID NO: 325 | R1765 | E-Sarbeco-2 | UAAUUUCUACUAAGUGUAGAUGU |
| | | | GGUAUUCUUGCUAGUUAC |
| SEQ ID NO: 326 | R1766 | CDC-N2-SARS | UAAUUUCUACUAAGUGUAGAUGU |
| | | | CCAAGUGCCUCUGCAUUC |
| SEQ ID NO: 327 | R1767 | N-Sarbeco-1 | UAAUUUCUACUAAGUGUAGAUCC |
| | | | AAUGUUGUUCCUUGAGGA |
| SEQ ID NO: 328 | R1768 | ORF1ab-Wuhan | UAAUUUCUACUAAGUGUAGAUCA |
| | | | CAUACCGCAGACGGUACA |
| SEQ ID NO: 329 | R1769 | CDC-RNaseP | UAAUUUCUACUAAGUGUAGAUGA |
| | | | CCUGCGAGCGGGUUCUGA |
| SEQ ID NO: 330 | R779 | RNaseP POP7 | UAAUUUCUACUAAGUGUAGAUAA |
| | | | UUACUUGGGUGUGACCCU |
| SEQ ID NO: 331 | R1965 | RNaseP POP7 v2 | UAAUUUCUACUAAGUGUAGAUUU |
| | | | ACAUGGCUCUGGUCCGAG |
| SEQ ID NO: 332 | R780 | RNaseP POP7 v3 | UAAUUUCUACUAAGUGUAGAUGG |
| | | | CTTCCAGGGAACAGGCCT |

### Example 34

### Limit of Detection of SARS-CoV-2

This example describes the limit of detection of SARS-CoV-2. Samples containing decreasing copy numbers of SARS-CoV-2 target nucleic acid were detected using a DETECTR reaction. FIG. 82 shows the results of a DETECTR reaction to determine the limit of detection of SARS-CoV-2 in a DETECTR reaction amplified using a primer set directed to the N-gene of SARS-CoV-2 (“2019-nCoV-N-set1”). Samples containing either 15,000, 4,000, 1,000, 500, 200, 100, 50, 20, or 0 copies of a SARS-CoV-2 N-gene target nucleic acid were detected. A gel of the N-gene RNA is shown below. Samples were detected using a gRNA directed to the N-gene of SARS-CoV-2 (SEQ ID NO: 323).

FIG. 116 shows DETECTR analysis of SARS-CoV-2 identifies down to 10 viral genomes in approximately 30 min (20 min amplification, 10 min DETECTR). Duplicate LAMP reactions were amplified for twenty min followed by LbCas12a DETECTR analysis.

FIG. 117 shows the raw fluorescence at 5 minutes for the LbCas12a DETECTR analysis provided in FIG. 116. The limit of detection of the SARS-CoV-2 N-gene was determined to be 10 viral genomes per reaction (n=6).

### Example 35

### Multiplexing SARS-CoV-2 Primer Sets for Detection of SARS-CoV-2

This example describes multiplexing SARS-CoV-2 primer sets for detection of SARS-CoV-2. Samples containing target nucleic acids were amplified using a combination of primer sets directed to one or more of SARS-CoV-2 or RNase P. Primer sets directed to SARS-CoV-2 are denoted by “set” with a number. FIG. 84 shows the time to result of a multiplexed DETECTR reaction. Samples contained either in vitro transcribed N-gene of SARS-CoV-2 (“N-gene IVT”), in vitro transcribed E-gene of SARS-CoV-2 (“E-gene IVT”), HeLa total RNA, or no target (“NTC”). Samples were amplified using one or more primer sets directed to the SARS-CoV-2 N-gene (“set1”), the SARS-CoV-2 E-gene (“set14”), or RNase” (“RNaseP”). FIG. 85 shows the time to results of a multiplexed DETECTR reaction with different combinations of primer sets directed to either SARS-CoV-2 N-gene (“set1”), SARS-CoV-2 E-gene (“set14”), or RNase P. Samples containing in vitro transcribed N-gene of SARS-CoV-2 (left, “N-gene IVT”) or in vitro transcribed E-gene of SARS-CoV-2 (right, “E-gene IVT”) were tested. FIG. 86 shows the time to result of a multiplexed DETECTR reaction with the best performing primer set combinations from FIG. 84 and FIG. 85. FIG. 84, at bottom, shows a series of line graphs of DETECTR reactions for different primer sets, which from left to right are 0.5× set1, 0.5× set14, 0.5× RNaseP, 0.25× set1, 0.25× set14, 0.25× RNaseP, 0.5× set1/RNaseP, 0.5× set1/set14/RNaseP, 0.5 set14/RNaseP, 0.25× set1/RNaseP, 0.25× set1/set14/RNaseP, 0.25× set14/RNaseP. The x-axis shows time in minutes ranging from 0 to 60 in increments of 10. The y-axis ranges from 0e+00 to 3e+07 in increments of 1e+07. Each line on the graph shows targets including N-gene IVT, E-gene IVT, HeLa total RNA, and non-target control (NTC). In each graph, the NTC line is essentially the lowest line and in some graphs is primarily flat. In the left most graph, the N-gene IVT line exhibits the highest signal most quickly. In the second from the leftmost graph, the E-gene IVT line exhibits the highest signal most quickly. In the third from the leftmost graph, the HeLa total RNA line exhibits the highest signal most quickly with the N-gene IVT and E-gene IVT lines exhibiting signal later on near 40-50 minutes. In the fourth from the leftmost graph, the N-gene IVT line exhibits the highest signal most quickly followed by the HeLa total RNA line exhibiting signal later on near 40-50 minutes. In the fifth from the leftmost graph, the E-gene IVT line exhibits the highest signal most quickly. In the sixth from the leftmost graph, the HeLa total RNA line exhibits the highest signal most quickly. In the seventh from the leftmost graph, the N-gene IVT and HeLa total RNA lines exhibit the highest signals most quickly. In the eight from the leftmost graph, the N-gene IVT, E-gene IVT, and HeLa total RNA lines exhibit the highest signals most quickly. In the ninth from the leftmost graph, the E-gene IVT and HeLa total RNA lines exhibit the highest signals most quickly. In the tenth from the leftmost graph, the N-gene IVT and HeLa total RNA lines exhibit the highest signal most quickly. In the eleventh from the leftmost graph, the N-gene IVT, E-gene IVT, and HeLa total RNA lines exhibit the highest signals most quickly. In the rightmost graph, the E-gene IVT and HeLa total RNA lines exhibit the highest signals most quickly.

FIG. 101 shows the results of a DETECTR assay evaluating LAMP primer sets for their utility in multiplexed amplification of SARS-CoV-2 targets. Samples were amplified with one or more primer sets directed to the SARS-CoV-2 N-gene (“set1”) or the SARS-CoV-2 E-gene (“set14”), or RNase P (“RNaseP”). Samples were detected with either a gRNA directed to the N-gene of SARS-CoV-2 (SEQ ID NO: 323, “N-gene”), the E-gene of SARS-CoV-2 (SEQ ID NO: 325, “E-gene”), or RNase P (SEQ ID NO: 330).

### Example 36

### Sensitivity of a DETECTR Assay to Distinguish Three Coronaviruses

This example describes the sensitivity of a DETECTR assay to distinguish three coronaviruses. Samples containing 250 pM of either RNA corresponding to the N-gene of SARS-CoV-2, the N-gene of SARS-CoV, or the N-gene of bat-SL-CoV45. Samples were amplified at detected as described in EXAMPLE 2. Samples were detected using each of a gRNA directed to the N-gene of SARS-CoV-2 (“R1763”), a gRNA directed to the N-gene of SARS-CoV (“R1766”), or a gRNA directed to the N-gene of a Sarbeco coronavirus (“R1767”). Sequences of the gRNAs used in this example are provided in TABLE 20. FIG. 87B schematically illustrates the sequence of a region of the SARS-CoV-2 N-gene (“N-Sarbeco”) target site. Target site sequences are provided in TABLE 21. FIG. 88 shows the results of a DETECTR assay to determine the sensitivity of gRNAs directed to either N-gene of SARS-CoV-2 (“R1763,” SEQ ID NO: 323), the N-gene of SARS-CoV (“R1766,” SEQ ID NO: 326), or the N-gene of a Sarbeco coronavirus (“R1767,” SEQ ID NO: 327) for samples containing either the N-gene of SARS-CoV-2 (“N-2019-nCoV”), the N-gene of SARS-CoV (“N-SARS-CoV”), or the N-gene of bat-SL-CoV45 (“N-bat-SL-CoV45”). SARS-CoV-2, SARS-CoV, and bat-SL-CoV45 are strains of sarbeco coronavirus. Samples were detected using LbCas12a (SEQ ID NO: 27). FIG. 88 shows line graphs of DETECTR reactions. The graphs from left to right show different targets including N-2019-nCoV, N-SARS-CoV, and N-bat-SL-CoV45. The x-axis shows time in minutes ranging from 0 to 75 in increments of 25. The y-axis shows raw fluorescence in arbitrary units (AU) ranging from 0 to 1000000 in increments of 250000. The three lines in each graph include R1763, R1766, and R1767. In the leftmost graph, the R1766 line appears flat while the R1763 and R1767 lines exhibit the highest signal most quickly. In the middle graph, the R1763 line appears flat while the R1766 and R1767 lines exhibit the highest signal most quickly. In the rightmost graph, the R1763 line appears flat while the R1766 and R1767 lines exhibit the highest signal most quickly.

FIG. 99 shows the results of a DETECTR assay evaluating multiple gRNAs for their utility in distinguishing between three different strains of coronavirus, SARS-CoV-2 (“COVID-2019”), SARS-CoV, or bat-SL-CoV45. Samples containing N-gene amplicons of either SARS-CoV-2 (“N-2019-nCoV”), SARS-CoV (“N-SARS-CoV”), or bat-SL-CoV45 (“N-bat-SL-CoV45”) were tested. Samples were detected with a gRNA directed to the N-gene of SARS-CoV-2 (SEQ ID NO: 323, “COVID-2019 gRNA”), a gRNA directed to the N-gene of SARS-CoV (SEQ ID NO: 326, “SARS-CoV gRNA”), or a gRNA directed to the N-gene of multiple coronavirus species (SEQ ID NO: 327, “multi-CoV gRNA”).

TABLE 21
Exemplary Coronavirus N-Gene and E-Gene Gene Fragments

| SEQ ID NO: | Target | Sequence |
|---|---|---|
| SEQ ID NO: | 2019- | CCAAATTGGCTACTACCGAAGAGCTACCAGACGAATTCGTGGT |
| 333 | nCoV | GGTGACGGTAAAATGAAAGATCTCAGTCCAAGATGGTATTTCTA |
| | N-gene | CTACCTAGGAACTGGGCCAGAAGCTGGACTTCCCTATGGTGCTA |
| | | ACAAAGACGGCATCATATGGGTTGCAACTGAGGGAGCCTTGAA |
| | | TACACCAAAAGATCACATTGGCACCCGCAATCCTGCTAACAAT |
| | | GCTGCAATCGTGCTACAACTTCCTCAAGGAACAACATTGCCAAA |
| | | AGGCTTCTACGCAGAAGGGAGCAGAGGCGGCAGTCAAGCCTCT |
| | | TCTCGTTCCTCATCACGTAGTCGCAACAGTTCAAGAAATTCAAC |
| | | TCCAGGCAGCAGTAGGGGAACTTCTCCTGCTAGAATGGCTGGC |
| | | AATGGCGGTGATGCTGCTCTTGCTTTGCTGCTGCTTGACAGATT |
| | | GAACCAGCTTGAGAGCAAAATGTCTGGTAAAGGCCAACAACAA |
| | | CAAGGCCAAACTGTCACTAAGAAATCTGCTGCTGAGGCTTCTAA |
| | | GAAGCCTCGGCAAAAACGTACTGCCACTAAAGCATACAATGTA |
| | | ACACAAGCTTTCGGCAGACGTGGTCCAGAACAAACCCAAGGAA |
| | | ATTTTGGGGACCAGGAACTAATCAGACAAGGAACTGATTACAA |
| | | ACATTGGCCGCAAATTGCACAATTTGCCCCCAGCGCTTCAGCGT |
| | | TCTTCGGAATGTCGCGCATTGGCATGGAAGTCACACCTTCGGGA |
| | | ACGTGGTTGACCTACACAGGTGCCATCAAATTGGATGACAAAG |
| | | ATCCAAATTTCAAAGATCAAGTCATTTTGCTGAATAAGCATATT |
| | | GACGCATACAAAACATTCCCACCAACAGAGCCTAAAAAGGACA |
| | | AAAAGAAGAAGGCTGATGAAACTCAAGCCTTACCGCAGAGACA |
| | | GAAGAAACAGCAAACTGTG |
| SEQ ID NO: | SARS- | CCAAATTGGCTACTACCGAAGAGCTACCCGACGAGTTCGTGGT |
| 334 | CoVN- | GGTGACGGCAAAATGAAAGAGCTCAGCCCCAGATGGTACTTCT |
| | gene | ATTACCTAGGAACTGGCCCAGAAGCTTCACTTCCCTACGGCGCT |
| | | AACAAAGAAGGCATCGTATGGGTTGCAACTGAGGGAGCCTTGA |
| | | ATACACCCAAAGACCACATTGGCACCCGCAATCCTAATAACAA |
| | | TGCTGCCACCGTGCTACAACTTCCTCAAGGAACAACATTGCCAA |
| | | AAGGCTTCTACGCAGAGGGAAGCAGAGGCGGCAGTCAAGCCTC |
| | | TTCTCGCTCCTCATCACGTAGTCGCGGTAATTCAAGAAATTCAA |
| | | CTCCTGGCAGCAGTAGGGGAAATTCTCCTGCTCGAATGGCTAGC |
| | | GGAGGTGGTGAAACTGCCCTCGCGCTATTGCTGCTAGACAGATT |
| | | GAACCAGCTTGAGAGCAAAGTTTCTGGTAAAGGCCAACAACAA |
| | | CAAGGCCAAACTGTCACTAAGAAATCTGCTGCTGAGGCATCTA |
| | | AAAAGCCTCGCCAAAAACGTACTGCCACAAAACAGTACAACGT |
| | | CACTCAAGCATTTGGGAGACGTGGTCCAGAACAAACCCAAGGA |
| | | AATTTCGGGGACCAAGACCTAATCAGACAAGGAACTGATTACA |
| | | AACATTGGCCGCAAATTGCACAATTTGCTCCAAGTGCCTCTGCA |
| | | TTCTTTGGAATGTCACGCATTGGCATGGAAGTCACACCTTCGGG |
| | | AACATGGCTGACTTATCATGGAGCCATTAAATTGGATGACAAA |
| | | GATCCACAATTCAAAGACAACGTCATACTGCTGAACAAGCACA |
| | | TTGACGCATACAAAACATTCCCACCAACAGAGCCTAAAAAGGA |
| | | CAAAAAGAAAAAGACTGATGAAGCTCAGCCTTTGCCGCAGAGA |
| | | CAAAAGAAGCAGCCCACTGTG |
| SEQ ID NO: | bat-SL- | CCAAATTGGCTACTACCGTAGAGCTACCAGACGAATTCGTGGTG |
| 335 | CoVZC | GTGACGGTAAAATGAAAGAGCTCAGCCCCAGATGGTATTTTTA |
| | 45 N- | CTATCTAGGAACTGGACCAGAAGCTGGACTTCCCTATGGTGCTA |
| | gene | ACAAAGAAGGCATCATATGGGTTGCAACTGAGGGAGCCTTAAA |
| | | CACACCGAAAGACCACATTGGCACCCGCAATCCTGCTAACAAT |
| | | GCTGCAATCGTGCTACAACTTCCTCAAGGAACAACATTGCCAAA |
| | | AGGCTTCTACGCAGAAGGGAGCAGAGGCGGCAGTCAAGCTTCT |
| | | TCACGCTCCTCATCACGTAGTCGCAACAGTTCAAGAAACTCAAC |
| | | TCCAGGCAGCAGTAGGGGAACTTCTCCTGCTAGAATGGCTGGC |
| | | AATGGCGGTGACACTGCTCTTGCTTTGCTGCTGCTAGATAGGTT |
| | | GAACCAGCTTGAGAACAAAGTATCTGGCAAAGGCCAACAACAA |
| | | CAGGGCCAAACTGTCACTAAGAAATCTGCTGCTGAGGCATCTA |
| | | AAAAGCCTCGCCAAAAACGTACTGCTACAAAACAGTACAACGT |
| | | CACTCAAGCATTTGGGAGACGTGGTCCAGAACAAACCCAAGGA |
| | | AATTTTGGGGACCAAGAATTAATCAGACAAGGAACTGATTACA |
| | | AACATTGGCCGCAAATTGCACAATTTGCTCCAAGTGCCTCTGCA |
| | | TTCTTTGGAATGTCACGCATTGGCATGGAAGTCACACCTTCGGG |
| | | AACATGGCTGACTTATCATGGAGCCATTAAATTGGATGACAAA |
| | | GATCCACAATTCAAAGATAACGTCATACTGCTGAATAAGCACA |
| | | TTGACGCATACAAAACATTCCCACCAACAGAGCCTAAAAAGGA |
| | | CAAAAAGAAAAAGGCTGATGAACTTCAGGCTTTACCGCAGAGA |
| | | CAGAAGAAACAACAAACTGTG |
| SEQ ID NO: | 2019- | ACTATTACCAGCTGTACTCAACTCAATTGAGTACAGACACTGGT |
| 336 | nCoV | GTTGAACATGTTACCTTCTTCATCTACAATAAAATTGTTGATGA |
| | E-gene | GCCTGAAGAACATGTCCAAATTCACACAATCGACGGTTCATCCG |
| | | GAGTTGTTAATCCAGTAATGGAACCAATTTATGATGAACCGACG |
| | | ACGACTACTAGCGTGCCTTTGTAAGCACAAGCTGATGAGTACG |
| | | AACTTATGTACTCATTCGTTTCGGAAGAGACAGGTACGTTAATA |
| | | GTTAATAGCGTACTTCTTTTTCTTGCTTTCGTGGTATTCTTGCTA |
| | | GTTACACTAGCCATCCTTACTGCGCTTCGATTGTGTGCGTACTG |
| | | CTGCAATATTGTTAACGTGAGTCTTGTAAAACCTTCTTTTTACGT |
| | | TTACTCTCGTGTTAAAAATCTGAATTCTTCTAGAGTTCCTGATCT |
| | | TCTGGTCTAAACGAACTAAATATTATATTAGTTTTTCTGTTTGGA |
| | | ACTTTAATTTTAGCCATGGCAGATTCCAACGGTACTATTACCGT |
| | | TGAAGAGCTTAAAAAGCTCCTTGAACAATGGAACCTAGTAATA |
| | | GGTTTCCTATTCCTTACATGGATT |
| SEQ ID NO: | SARS- | TTTACTACCAGCTTGAGTCTACACAAATTACTACAGACACTGGT |
| 337 | CoV E- | ATTGAAAATGCTACATTCTTCATCTTTAACAAGCTTGTTAAAGA |
| | gene | CCCACCGAATGTGCAAATACACACAATCGACGGCTCTTCAGGA |
| | | GTTGCTAATCCAGCAATGGATCCAATTTATGATGAGCCGACGAC |
| | | GACTACTAGCGTGCCTTTGTAAGCACAAGAAAGTGAGTACGAA |
| | | CTTATGTACTCATTCGTTTCGGAAGAAACAGGTACGTTAATAGT |
| | | TAATAGCGTACTTCTTTTTCTTGCTTTCGTGGTATTCTTGCTAGT |
| | | CACACTAGCCATCCTTACTGCGCTTCGATTGTGTGCGTACTGCT |
| | | GCAATATTGTTAACGTGAGTTTAGTAAAACCAACGGTTTACGTC |
| | | TACTCGCGTGTTAAAAATCTGAACTCTTCTGAAGGAGTTCCTGA |
| | | TCTTCTGGTCTAAACGAACTAACTATTATTATTATTCTGTTTGGA |
| | | ACTTTAACATTGCTTATCATGGCAGACAACGGTACTATTACCGT |
| | | TGAGGAGCTTAAACAACTCCTGGAACAATGGAACCTAGTAATA |
| | | GGTTTCCTATTCCTAGCCTGGATT |
| SEQ ID NO: | bat-SL- | ATTACTACCAGCTGTACTCAACACAAGTGAGTACAGACACTGGT |
| 338 | CoVZC | GTTGAACATGTTACTTTCTTCATCTACAATAAAATTGTTGATGA |
| | 45 E- | GCCTGAAGAACATGTTCAAATTCACACAATCGACGGTACATCTG |
| | gene | GAGTTGTTAATCCAGCAATGGAACCAATTTATGATGAACCGAC |
| | | GACGACTACTAGCGTGCCTTTGTAAGCACAAGCTGATGAGTAC |
| | | GAACTTATGTACTCATTCGTTTCGGAAGAGACAGGTACGTTAAT |
| | | AGTTAATAGCGTACTTCTTTTTCTTGCTTTTGTGGTATTCTTGCT |
| | | AGTCACACTAGCCATCCTTACTGCGCTTCGATTGTGTGCGTACT |
| | | GCTGCAATATTGTTAACGTGAGTCTTGTAAAACCTTCTTTTTACG |
| | | TTTACTCTCGTGTTAAAAATCTGAATTCTTCTAGAGTTCCTGATC |
| | | TTTTGGTCTAAACGAACTAAATATTATATTAGTCTTTCTGTTTGG |
| | | AACTTTAATTTTAGCCATGTCAGGTGACAACGGTACCATTACCG |
| | | TTGAAGAGCTTAAAAAGCTCTTAGAACAATGGAACCTAGTAAT |
| | | AGGATTCTTGTTTCTTACATGGATT |

### Example 37

### Sensitivity of Detection of the E-Gene of Four Coronaviruses

This example describes the sensitivity of detection of the E-gene of three coronaviruses. Samples containing 250 pM of either RNA corresponding to the E-gene of SARS-CoV-2, the E-gene of SARS-CoV, the E-gene of bat-SL-CoV45, or the E-gene of bat-SL-CoV21. Samples were amplified at detected as described in EXAMPLE 2. Samples were detected using each of a first gRNA directed to the E-gene (R1764), or a second gRNA directed to the E-gene (R1765). Sequences of the gRNAs used in this example are provided in TABLE 20. FIG. 89 schematically illustrates the sequence of a region of the SARS-CoV-2 E-gene (“E-Sarbeco”) target site. Target site sequences are provided in TABLE 21. FIG. 90 shows the results of a DETECTR assay to determine the sensitivity of two gRNAs directed to a coronavirus N-gene for samples containing either the E-gene of SARS-CoV-2 (“E-2019-nCoV”), the E-gene of SARS-CoV (“E-SARS-CoV”), the E-gene of bat-SL-CoV45 (“E-bat-SL-CoV45”), or the E-gene of bat-SL-CoV21 (“E-bat-SL-CoV21”). Samples were detected with LbCas12a (SEQ ID NO: 27) and either a gRNA corresponding to SEQ ID NO: 324 (“R1764—E gene 1”) or a gRNA corresponding to SEQ ID NO: 325 (“R1765—E gene 2”). Fluorescence intensity was measured over time.

FIG. 100 shows the results of a DETECTR assay evaluating multiple gRNAs for their utility in distinguishing between three different strains of coronavirus, SARS-CoV-2 (“COVID-2019”), SARS-CoV, or bat-SL-CoV45. Samples containing E-gene amplicons of either SARS-CoV-2 (“N-2019-nCoV”), SARS-CoV (“N-SARS-CoV”), or bat-SL-CoV45 (“N-bat-SL-CoV45”) were tested. Samples were detected using gRNAs directed to the E-gene of multiple coronaviruses corresponding to SEQ ID NO: 324 (“E-gene gRNA #1”) or SEQ ID NO: 325 (“E-gene gRNA #2”). Detection of a sample with a gRNA directed to the E-gene enabled broad spectrum targeting of related coronavirus strains.

### Example 38

### Detection of a Coronavirus Using a Lateral Flow DETECTR Reaction using a Cas12 Variant

This example describes the detection of a coronavirus using a lateral flow DETECTR reaction. FIG. 106 illustrates the design of detector nucleic acids compatible with a PCRD lateral flow device. Exemplary compatible detector nucleic acids, rep072, rep076, and rep100, are provided (left). These detector nucleic acids may be used in a PCRD lateral flow device (right) to detect the presence or absence of a target nucleic acid. The top right schematic illustrates an exemplary band configuration produced when contacted to a sample that does not contain a target nucleic acid. The bottom right schematic shows an exemplary band configuration produced when contacted to a sample that does contain a target nucleic acid. Exemplary reporters compatible with a PCRD lateral flow device are provided in TABLE 22. The lateral flow cleavage reporter Rep100 enables detection of a sample on a lateral flow strip with application of the signal lines. The Rep072 reporter only gives a signal on the IgG line following cleavage of the reporter by a programmable nuclease. Similar to the rep076 reporter, which is attached to magnetic beads, the rep100 reporter generates a signal at the FAM-Biotin line on the PCRD strip when cleaved. However, unlike rep076, the rep100 reporter is captured at the DIG-biotin line, which eliminates the need for magnetic beads.

A sample comprising an RNA target sequence from a coronavirus was amplified using isothermal amplification. Samples containing either 0 fM (“−”) or 5 fM (“+”) of in vitro transcribed coronavirus N-gene were amplified for 60 minutes using a reverse-transcription LAMP (RT-LAMP) amplification assay. A DETECTR reaction was performed using a Cas12 variant (SEQ ID NO: 37) for either 0 min, 2.5 min, 5 min, or 10 min. FIG. 91 shows the results of a lateral flow DETECTR reaction to detect the presence or absence of a SARS-CoV-2 N-gene target RNA using a Cas12 variant (SEQ ID NO: 37). Lateral flow test strips are shown. Samples either containing (“+”) or lacking (“−”) in vitro transcribed SARS-CoV-2 N-gene RNA (“N-gene IVT”) were tested. The top set of horizontal lines (denoted “test”) indicated the results of the DETECTR reaction. The DETECTR reaction was sensitive for samples containing the in vitro transcribed coronavirus target sequence.

TABLE 22
Exemplary Reporter Sequences for Detection of Coronaviruses

| Reporter | Sequence |
|---|---|
| Rep072 | /56-FAM/TTATTATT/3Bio/ (SEQ ID NO: 9) |
| Rep076 | /56-FAM/*/iBiodT/*AATTAATTAATTAATTAATT/3ThioMC3-D/ (SEQ ID NO: 372) |
| Rep100 | /56-FAM/*/iBiodT/*AATTAATTAATTAATTAATT/3DiG_N/ (SEQ ID NO: 372) |

### Example 39

### Detection of SARS-CoV-2 Using a Lateral Flow DETECTR Reaction

This example describes the detection of SARS-CoV-2 using a lateral flow DETECTR reaction. FIG. 92 illustrates schematically the detection of a target nucleic acid using a programmable nuclease. Briefly, a Cas protein with trans collateral cleavage activity is activated upon binding to a guide nucleic acid and a target sequence reverse complementary to a region of the guide nucleic acid. The activated programmable nuclease cleaves a reporter nucleic acid, thereby producing a detectable signal. FIG. 93 illustrates schematically detection of the presence or absence of a target nucleic acid in a sample. Select nucleic acids in a sample are amplified using isothermal amplification. The amplified sample is contacted to a programmable nuclease, a guide nucleic acid, and a reporter nucleic acid, as illustrated in FIG. 92. If the sample contains the target nucleic acid, a detectable signal is produced. The presence or absence of a target nucleic acid corresponding to SARS-CoV-2 was detected using a DETECTR reaction following in vitro transcription and isothermal pre-amplification of the target nucleic acid. Samples were detected using a Cas12 programmable nuclease. Samples contained either SARS-CoV-2 viral RNA or a sequence corresponding to RNase P (negative control). Samples were detected using a gRNA directed to SARS-CoV-2 using the DETECTR reaction described in FIG. 92 and FIG. 93. FIG. 94 shows the results of a DETECTR lateral flow reaction to detect the presence or absence of SARS-CoV-2 (“2019-nCoV”) RNA in a sample. Detection of RNase P is used as a sample quality control. Samples were in vitro transcribed and amplified (left) and detected using a Cas12 programmable nuclease (right). Samples containing (“+”) or lacking (“−”) in vitro transcribed SARS-CoV-2 RNA (“2019-nCoV IVT”) were assayed with a Cas12 programmable nuclease and gRNA directed to SARS-CoV-2 for either 0 min or 5 min. The reaction was sensitive for samples containing SARS-CoV-2.

### Example 40

### Testing Clinical Samples for SARS-CoV-2 Using a DETECTR Reaction

This example describes the testing of clinical samples for SARS-CoV-2 using a DETECTR reaction. Clinical samples were amplified using RT-PCR and detected using LbCas12a. Samples were detected using gRNA (“crRNA”) directed to either the N-gene or the E-gene of SARS-CoV-2 or RNase P (negative control). FIG. 95 shows the results of a DETECTR reaction using an LbCas12a programmable nuclease (SEQ ID NO: 27) to determine the presence or absence of SARS-CoV-2 in patient samples. At the right of FIG. 95 are a set of bar graphs. The bar graph on top shows RT-PCR amplicon testing—15 minutes with the crRNA being varied on the x-axis and from left to right show crRNA for the N gene, E gene and RNase P. The y-axis shows raw fluorescence in arbitrary units (AU) ranging from 0 to 60000 in increments of 20000. Within each crRNA group are the different targets tested, which from left to right are sample 1, sample 1 NTC, sample 2, sample 2 NTC, and DETECTR NTC. The bar graph on bottom shows RT-PCR amplicon testing—15 minutes with the target being varied on the x-axis and from left to right show targets of sample 1, sample 1 NTC, sample 2, sample 2 NTC, and DETECTR NTC. The y-axis shows raw fluorescence in arbitrary units (AU) ranging from 0 to 60000 in increments of 20000. Within each target group are different crRNAs tested, which from left to right are N gene, E gene, and RNase P.

Clinical samples of patients either positive or negative for SARS-CoV-2 were assayed using a lateral flow DETECTR reaction. Samples were amplified and reverse transcribed using RT-PCR and detected using a Cas12 programmable nuclease. A negative control sample (“NTC”) was also assayed. The DETECTR reaction was performed for 5 min. FIG. 96 shows the results of a lateral flow DETECTR reaction to detect the presence or absence of SARS-CoV-2 in patient samples. Samples were detected with either a gRNA directed to SARS-CoV-2 or a gRNA directed to RNase P. Primers directed to a region of the E-gene were used to amplify the target region using RT-PCR.

### Example 41

### Buffer Screening for Improved RT-LAMP Amplification and Detection

This example describes buffer screening for improved RT-LAMP amplification and detection. Samples containing either HeLa total RNA (“total RNA”), SARS-CoV-2 N-gene RNA and HeLa total RNA (“N-gene +total RNA”) or no target (“NTC”) were amplified using RT-LAMP under different buffer conditions.

FIG. 102 shows the results of a DETECTR assay evaluating the sensitivity of an RT-LAMP amplification reaction to common sample buffers. Reactions were measured in universal transport medium (UTM, top) or DNA/RNA Shield buffer (bottom) at different buffer dilutions (from left to right: 1×, 0.5×, 0.25×, 0.125×, or no buffer).

### Example 42

### Limit of Detection of SARS-CoV-2 in a DETECTR Assay

This example describes the limit of detection of SARS-CoV-2 in a DETECTR assay. DETECTR reactions were performed with different copy numbers of SARS-CoV-2 viral genomes. FIG. 103 shows the results of a DETECTR assay to determine the limit of detection (LoD) of the DETECTR assay for SARS-CoV-2 (the virus attributed to the COVID-19 infection). Samples were detected using either a gRNA directed to the N-gene of SARS-CoV-2 (SEQ ID NO: 323, “R1763—N-gene”) or a gRNA directed to RNase P (SEQ ID NO: 330, “R779—RNase P”). Each condition was repeated 7 times. The DETECTR assay was capable of reproducibly and specifically detecting the presence of SARS-CoV-2 RNA down to between about 625 and about 150 copies per reaction.

### Example 43

### Target Specificity of a Multiplexed RT-LAMP Amplification with DETECTR

This example describes the target specificity of a multiplexed RT-LAMP amplification with DETECTR reaction. FIG. 104 shows the results of a DETECTR assay evaluating the target specificity of a gRNA directed to the N-gene of SARS-CoV-2 (“R1763—N-gene”) in a 2-plex multiplexed RT-LAMP reaction using an LbCas12a programmable nuclease (SEQ ID NO: 27). In vitro transcribed coronavirus N-gene sequences from either SARS-CoV-2 (“2019-nCoV N-gene IVT), SARS-CoV (“SARS-CoV N-gene IVT”), or bat-SL-CoV45 (“bat-SL-CoV45 N-gene IVT”) or clinical remnant samples from patients having different strains of coronavirus (CoV-HKU1, CoV-299E, CoV-OC43, or CoV-NL63) were amplified using a 2-plex multiplexed RT-LAMP amplification. HeLa total RNA was used as a positive control for RNase P. A no target control (“NTC”) was tested as a negative control. The 2-plex multiplexed RT-LAMP amplification amplified the samples using two primer sets, one directed to the SARS-CoV-2 N-gene and one directed to RNaseP. Amplified samples were detected using either a gRNA directed to RNase P (SEQ ID NO: 330, “R779—RNase P”) or the N-gene of SARS-CoV-2 (SEQ ID NO: 323, “R1763—N-gene”). Both gRNAs were capable of detecting samples amplified in a 2-plex multiplexed RT-LAMP amplification assay.

FIG. 105 shows the results of a DETECTR assay evaluating the target specificity of a gRNA directed to the N-gene of SARS-CoV-2 (“R1763—N-gene”) or the E-gene of SARS-CoV-2 (“R1765—E-gene”) in a 3-plex multiplexed RT-LAMP reaction using an LbCas12a programmable nuclease (SEQ ID NO: 27). In vitro transcribed coronavirus N-gene sequences from either SARS-CoV-2 (“2019-nCoV N-gene IVT), SARS-CoV (“SARS-CoV N-gene IVT”), or bat-SL-CoV45 (“bat-SL-CoV45 N-gene IVT”), in vitro transcribed coronavirus E-gene sequences from SARS-CoV-2 (“2019-nCoV E-gene IVT) or SARS-CoV (“SARS-CoV E-gene IVT”), or clinical remnant samples from patients having different strains of coronavirus (CoV-HKU1, CoV-299E, CoV-OC43, or CoV-NL63) were amplified using a 3-plex multiplexed RT-LAMP amplification. HeLa total RNA was used as a positive control for RNase P. A no target control (“NTC”) was tested as a negative control. The 3-plex multiplexed RT-LAMP amplification amplified the samples using three primer sets, one directed to the SARS-CoV-2 N-gene, one directed to the SARS-CoV-2 E-gene, and one directed to RNaseP. Amplified samples were detected using either a gRNA directed to RNase P (SEQ ID NO: 330, “R779—RNase P”), the N-gene of SARS-CoV-2 (SEQ ID NO: 323, “R1763—N-gene”), or the E-gene of SARS-CoV-2 (SEQ ID NO: 325, “R1765—E-gene”). All three gRNAs were capable of detecting samples amplified in a 3-plex multiplexed RT-LAMP amplification assay.

### Example 44

### Coronavirus Strain Specificity of N-Gene and E-Gene gRNAs

This example describes coronavirus strain specificity of N-gene and E-gene gRNAs. Guide RNAs were designed to specifically detect the N-gene of SARS-CoV-2. Guide RNAs were also designed to detect the E-gene in three SARS-like coronavirus strains (SARS-CoV, bat SARS-like coronavirus (bat-SL-CoVZC45), and SARS-CoV-2). Synthetic in vitro transcribed (IVT) SARS-CoV-2 RNA gene targets were spiked into nuclease-free water. Samples were detected with a CRISPR-Cas12 based detection assay using LbCas12a (SEQ ID NO: 27). DETECTR assays included an RT-LAMP reaction at 62° C. for 20 min and Cas12 detection reaction at 37° C. for 10 min. Primers for target generation, qPCR, and LAMP amplification are provided in TABLE 23. FIG. 107A illustrates a genome map indicating the locations of the E (envelope) gene and the N (nucleoprotein) gene regions within a coronavirus genome. Corresponding regions or annealing regions of primers and probes relative to the E and N gene regions are shown below the respective gene regions. RT-LAMP primers are indicated by black rectangles, the binding position of the F1c and B1c half of the FIP primer (grey) is represented by a striped rectangle with dashed borders. Regions amplified in tests utilized by the World Health Organization (WHO) and the Center for Disease Control (CDC) are denoted as “WHO E amplicon” and “CDC N2 amplicon,” respectively.

Guide RNAs were able to distinguish SARS-CoV-2 without cross-reactivity with related coronavirus strains using the N gene gRNA and with the expected cross-reactivity for the E gene gRNA. FIG. 107B shows the results of a DETECTR assay evaluating the specificity or broad detection utility of gRNAs directed to the N-gene or E-gene of various coronavirus strains (SARS-CoV-2, SARS-CoV, or bat-SL-CoVZC45) using an LbCas12a programmable nuclease (SEQ ID NO: 27). The N gene gRNA used in the assay (left, “N-gene”) was specific for SARS-CoV-2, whereas the E gene gRNA was able to detect 3 SARS-like coronavirus (right, “E-gene”). A separate N gene gRNA targeting SARS-CoV and a bat coronavirus failed to detect SARS-CoV-2 (middle, “N-gene related species variant”). Guide RNAs were designed to specifically target SARS-CoV-2 or broadly detect related coronavirus strains. Samples containing either SARS-CoV-2 N-gene (“N-gene: SARS-CoV-2”), SARS-CoV N-gene (“N-gene: SARS-CoV”), bat-SL-CoVZC45 N-gene (“N-gene: bat-SL-CoVZC45”), SARS-CoV-2 E-gene (“E-gene: SARS-CoV-2”), SARS-CoV E-gene (“E-gene: SARS-CoV”), or bat-SL-CoVZC45 E-gene (“E-gene: bat-SL-CoVZC45”) were detected using either a gRNA designed to specifically detect the SARS-CoV N-gene (SEQ ID NO: 323, “N-gene”), a gRNA designed to detect the N-gene of coronavirus variants (SEQ ID NO: 326, “N-gene (related species variant)”), or a gRNA designed to broadly detect coronavirus E-gene (SEQ ID NO: 324, “E-gene”).

TABLE 23
Target Generation and Amplification Primers

| SEQ ID NO: | Name | Sequence | Purpose |
|---|---|---|---|
| SEQ ID NO: 340 | N-gene-FWD IVT | AATTCTAATACGACTCACTATAGGGCC | Target |
| | | AAATTGGCTACTACCGAAGAGCTAC | Generation |
| SEQ ID NO: 341 | N-gene-REV IVT | CACAGTTTGCTGTTTCTTCTGTCTCTGC | Target |
| | | GG | Generation |
| SEQ ID NO: 342 | E-gene-FWD IVT | AATTCTAATACGACTCACTATAGGGCT | Target |
| | | GGTGTTGAACATGTTACCTTCTTCATC | Generation |
| SEQ ID NO: 343 | E-gene-REV IVT | CCTATTACTAGGTTCCATTGTTC | Target |
| | | | Generation |
| SEQ ID NO: 344 | E_Sarbeco_F1 | ACAGGTACGTTAATAGTTAATAGCGT | qPCR |
| SEQ ID NO: 345 | E_Sarbeco_R2 | ATATTGCAGCAGTACGCACACA | qPCR |
| SEQ ID NO: 346 | CDC N2-FWD | TTACAAACATTGGCCGCAAA | qPCR |
| SEQ ID NO: 347 | CDC N2-REV | GCGCGACATTCCGAAGAA | qPCR |
| SEQ ID NO: 348 | F3 2019-nCoVN- | AACACAAGCTTTCGGCAG | LAMP |
| | gene | | |
| SEQ ID NO: 349 | B3 2019-nCoVN- | GAAATTTGGATCTTTGTCATCC | LAMP |
| | gene | | |
| SEQ ID NO: 350 | BIP 2019-nCoV | TGCGGCCAATGTTTGTAATCAGCCAAG | LAMP |
| | N-gene | GAAATTTTGGGGAC | |
| SEQ ID NO: 351 | FIP 2019-nCoV | CGCATTGGCATGGAAGTCACTTTGATG | LAMP |
| | N-gene | GCACCTGTGTAG | |
| SEQ ID NO: 352 | LF 2019-nCoVN- | TTCCTTGTCTGATTAGTTC | LAMP |
| | gene | | |
| SEQ ID NO: 353 | LB 2019-nCoVN- | ACCTTCGGGAACGTGGTT | LAMP |
| | gene | | |
| SEQ ID NO: 354 | F3 2019-nCoV E- | CCGACGACGACTACTAGC | LAMP |
| | gene | | |
| SEQ ID NO: 355 | B3 2019-nCoV E- | AGAGTAAACGTAAAAAGAAGGTT | LAMP |
| | gene | | |
| SEQ ID NO: 356 | BIP 2019-nCoV | ACCTGTCTCTTCCGAAACGAATTTGTA | LAMP |
| | E-gene | AGCACAAGCTGATG | |
| SEQ ID NO: 357 | FIP 2019-nCoV E- | CTAGCCATCCTTACTGCGCTACTCACG | LAMP |
| | gene | TTAACAATATTGCA | |
| SEQ ID NO: 358 | LF 2019-nCoV E- | TCGATTGTGTGCGTACTGC | LAMP |
| | gene | | |
| SEQ ID NO: 359 | LB 2019-nCoV E- | TGAGTACATAAGTTCGTAC | LAMP |
| | gene | | |
| SEQ ID NO: 360 | F3 RNase P POP7 | TTGATGAGCTGGAGCCA | LAMP |
| SEQ ID NO: 361 | B3 RNase P POP7 | CACCCTCAATGCAGAGTC | LAMP |
| SEQ ID NO: 362 | FIP RNase P | GTGTGACCCTGAAGACTCGGTTTTAGC | LAMP |
| | POP7 | CACTGACTCGGATC | |
| SEQ ID NO: 363 | BIP RNase P | CCTCCGTGATATGGCTCTTCGTTTTTTT | LAMP |
| | POP7 | CTTACATGGCTCTGGTC | |
| SEQ ID NO: 364 | LF RNase P POP7 | ATGTGGATGGCTGAGTTGTT | LAMP |
| SEQ ID NO: 365 | LB RNase P POP7 | CATGCTGAGTACTGGACCTC | LAMP |
| SEQ ID NO: 366 | F3 RNase P POP7 | CACATCCGAGTCTTCAGG | LAMP |
| | v2 | | |
| SEQ ID NO: 367 | B3 RNase P POP7 | GGCAATAGTTACAGACCGC | LAMP |
| | v2 | | |
| SEQ ID NO: 368 | FIP RNase P | TCCAGTACTCAGCATGCGAAGCACCCA | LAMP |
| | POP7 v2 | AGTAATTGAAAAGACAC | |
| SEQ ID NO: 369 | BIP RNase P | CTGGAAGCCCAAAGGACTCTATACACA | LAMP |
| | P0P7 v2 | CACTCAGGAAGG | |
| SEQ ID NO: 370 | LF RNase P POP7 | CGGAGGGGATAAGTGGAGGA | LAMP |
| | v2 | | |
| SEQ ID NO: 371 | LB RNase P POP7 | GCATTGAGGGTGGGGGT | LAMP |
| | v2 | | |

### Example 45

### Specific and Broad Detection of Coronaviruses using a Lateral Flow DETECTR Assay

This example describes specific and broad detection of coronaviruses using a lateral flow DETECTR assay. Lateral flow DETECTR assays can be performed with minimal equipment within appropriate biosafety laboratory requirements. FIG. 107C shows exemplary laboratory equipment utilized in the coronavirus lateral flow DETECTR assays. In addition to appropriate biosafety protective equipment, the equipment utilized includes a sample collection device, microcentrifuge tubes, heat blocks set to 37° C. and 62° C., pipettes and tips, and lateral flow strips.

The DETECTR assay can be run within 30 to 40 minutes and visualized on a lateral flow strip. Conventional RNA extraction or sample matrix can be used as an input to DETECTR (LAMP pre-amplification and Cas12-based detection for N gene, E gene and RNase P), which is visualized by a fluorescent reader or lateral flow strip. The SARS-CoV-2 DETECTR assay was considered positive if there was detection of both the E and N genes, or presumptive positive if there was detection of either the E or N gene. This interpretation is consistent with that of current FDA Emergency Use Authorization (EUA) guidance and recently approved point-of-care diagnostics under the EUA. FIG. 107D illustrates an exemplary workflow of a DETECTR assay for the detection of a coronavirus in a subject. Patient samples are collected using a nasopharyngeal swab. Conventional RNA extraction or sample matrix can be used as an input to DETECTR (LAMP pre-amplification and Cas12-based detection for NE gene, EN gene and RNase P), which is visualized by a fluorescent reader or lateral flow strip. Samples can be detected directly from the raw sample matrix, or the viral RNA can be extracted and then detected. Viral RNA encoding SARS-CoV-2 E-gene and SARS-CoV N-gene and RNA encoding human RNase P is amplified using an isothermal amplification method such as RT-LAMP. Amplified samples are detected using a Cas12 programmable nuclease complexed with gRNAs directed to SARS-CoV-2 N-gene and E-gene sequences. The Cas12 programmable nuclease cleaves a ssDNA reporter nucleic acid upon complex formation with the target nucleic acid. The sample is then detected using a lateral flow readout. Sample collection may be performed in about 0 min to about 10 min, amplification and detection may be performed in about 20 min to about 30 min, and sample readout may be performed in about 2 min.

FIG. 107E shows lateral flow test strips (left) indicating a positive test result for SARS-CoV-2 N-gene (left, top) and a negative test result for SARS-CoV-2 N-gene (left, bottom). A positive identification SARS-CoV-2 in a sample required detection of both the E-gene and the N-gene to confirm a positive test. The lateral assay was performed as illustrated and described in FIG. 107D. The table (right) illustrates possible test indicators and associated results for a lateral flow strip-based coronavirus diagnostic assay that tests for the presences of absence of the RNaseP (positive control), SARS-CoV-2 N-gene, and coronavirus E-gene. Detection of the two SARS-CoV-2 viral gene targets and the internal spiked human RNase P control indicates a positive result.

### Example 46

### Amplification and Detection of Patient Samples Directly from Raw Sample Matrix

This example describes amplification and detection of patient samples directly from raw sample matrix. The capability of the RT-LAMP assay to amplify SARS-CoV-2 nucleic acid directly from raw sample matrix was assessed. Samples consisting of nasal swabs from asymptomatic donors placed in universal transport medium (UTM) or phosphate buffered saline (PBS) and spiked with SARS-CoV-2 IVT target RNA were assayed using RT-LAMP DETECTR reactions. Since nasal swabs are more frequently collected in universal transport medium (UTM) than in phosphate buffered saline (PBS), the effect of running the assay from nasal swab sample matrix consisting of UTM buffer was evaluated. Nasal swabs from asymptomatic donors were collected in UTM or PBS.

FIG. 110A shows the time to result of an RT-LAMP amplification under different buffer conditions. Time to results was calculated as the time at which the fluorescent value is one third of the max for the experiment. Reactions that failed to amplify are reported with a value of 20 minutes and labeled as “no amp.” Time to result was determined for different starting concentrations of target control plasmid in either water, 10% phosphate buffered saline (PBS), or 10% universal transport medium (UTM). A lower time to result indicates faster amplification. Results indicate that 10% PBS inhibits the assay less than 10% UTM. FIG. 110B shows the results of an RT-LAMP assay to determine the amplification efficiency of the N-gene of SARS-CoV-2, the E-gene of SARS-CoV-2, and RNase P in either 5% UTM, 5% PBS, or water. Samples containing 0.5 fM N-gene in vitro transcribed, 0.5 fM of E-gene in vitro transcribed, and 0.8 ng/μL HeLa total RNA (“N+E+total RNA”) or no target controls (“NTC”) were tested. Evaluation of amplification efficiency for RT-LAMP for the N-gene, E-gene, and RNase P in 5% sample buffer final volume showed that RT-LAMP was functional for all target genes at a 5% sample buffer concentration. Final target concentrations were at 0.5 fM N-gene IVT, 0.5 fM E-gene IVT, and 0.08 ng/μL HeLa total RNA. FIG. 110C shows amplification of RNA directly from nasal swabs in PBS. Time to result was measured as a function of PBS concentration. Nasal swabs (“nasal swab”) were either spiked with HeLa total RNA (left, “total RNA: 0.08 ng/uL”) or water (right, “total RNA: 0 ng/uL”). Samples without a nasal swab (“no swab”) were compared as controls. With RT-LAMP, assay performance degraded at reaction concentrations of ≥10% UTM by volume or ≥20% PBS by volume. The estimated limit of detection decreased to 500 copies/μL in ≥10% UTM and to 1,00 copies/μL in ≥20% PBS. RT-LAMP was capable of amplifying RNA directly from nasal swabs in PBS with the best performance at 5% or 10% final volume of PBS per RT-LAMP pre-amplification reaction. Nasal swabs were prepared in PBS and either spiked with HeLa total RNA or water and run at various concentrations in an RT-LAMP reaction for RNase P.

### Example 47

### Limit of Detection of a DETECTR Assay for SARS-CoV-2

This example describes the limit of detection of a DETECTR assay for SARS-CoV-2. Using IVT SARS-CoV-2 target RNA spiked into donor nasal swab sample matrix in PBS, the analytic limits of detection (LoD) of the DETECTR assay was compared relative to the US FDA Emergency Use Authorization (EUA)-approved CDC assay (running tests for 2 of the 3 targets, N2 and N3) for detection of SARS-CoV-2. Five 10-fold serial dilutions of in vitro transcribed viral RNA were spiked into sample matrix at concentrations ranging from 101-105 copies/mL, with 6 replicates at each dilution for the DETECTR assay, and 3 replicates at each dilution for the CDC assay. FIG. 111A shows raw fluorescence curves generated by LbCas12a (SEQ ID NO: 27) detection of SARS-CoV-2 N-gene (n=6). The curves showed saturation in less than 20 minutes. FIG. 111B shows the limit of detection of a DETECTR assay for the SARS-CoV-2 N-gene detected with LbCas12a, as determined from the raw fluorescence traces shown in FIG. 111A. Fluorescence intensity was measured with decreasing concentration (copies per mL) of SARS-CoV-2 N-gene. FIG. 111C shows the time to result of the limit of detection DETECTR assay, as determined from the raw fluorescence traces shown in FIG. 111A. A lower time to result indicated faster amplification and detection. The estimated LoD for SARS-CoV-2 DETECTR was approximately 10 copies/μl, which is comparable to the LoD for the CDC N2 and N3 assays. DETECTR analysis of SARS-CoV-2 identified down to 10 viral genomes in less than 30 minutes. Duplicate LAMP reactions were amplified for twenty minutes followed by LbCas12a DETECTR analysis. Further analysis reveals the limit of detection of the SARS-CoV-2 N-gene to be 10 viral genomes per reaction (n=6, FIG. 111B). Evaluation of the time to result of these reactions highlights detection of 10 viral genomes of SARS-CoV-2 in under 5 minutes (n=6, FIG. 111C).

The analytic limit of detection of the RT-LAMP DETECTR reaction was compared relative to the qRT-PCR detection assay used by the US FDA Emergency Use Authorization-approved CDC assay for detection of SARS-CoV-2. A standard curve for quantitation was constructed using 7 dilutions of a control IVT viral nucleoprotein RNA (“CDC VTC nCoV Transcript”), with 3 replicates at each dilution, and detected using the CDC protocol (FIG. 108D, left). Ten two-fold serial dilutions of the same control nucleoprotein RNA were then used to run the DETECTR assay, with 6 replicates at each dilution (FIG. 108D, middle). The estimated limit of dilution for the CDC assay tested by California Department of Public Health was 1 copy/μL reaction, consistent with the analytic performance in the FDA package insert, versus 10 copies/μL reaction for the DETECTR assay. FIG. 108D shows the results of a DETECTR assay with LbCas12a (middle) or a CDC protocol (left) to determine the limit of detection of SARS-CoV-2. Signal is shown as a function of the number of copies of viral genome per reaction. Representative lateral flow results for the assay shown for 0 copies/μL and 10 copies/μL (right).

The limit of detection (LoD) was measured for detection of SARS-CoV-2 using a lateral flow device. FIG. 108A illustrates cleavage of a detector nucleic acid labeled with FAM and biotin by a Cas12 programmable nuclease in the presence of a target nucleic acid (top). Schematics of lateral flow test strips (bottom) illustrate markings indicative of either the presence (“positive”) or absence (“negative) of the target nucleic acid in the tested sample. The intact FAM-biotinylated reporter molecule flows to the control capture line. Upon recognition of the matching target, the Cas-gRNA complex cleaves the reporter molecule, which flows to the target capture line.

### Example 48

### Effects Incubation Time in a DETECTR Assay for SARS-CoV-2

This example describes the effects of incubation time in a DETECTR assay for SARS-CoV-2. Samples were amplified using RT-LAMP and detected using LbCas12a (SEQ ID NO: 27). The effect of the Cas12 reaction incubation time on signal was tested.

FIG. 108B shows the results of a DETECTR assay using LbCas12a to determine the effect of reaction time for a sample containing either 0 fM SARS-CoV-2 RNA or 5 fM SARS-CoV-2 RNA. Fluorescence signal of LbCas12a detection assay on RT-LAMP amplicon for SARS-CoV-2 N-gene saturated within 10 minutes. RT-LAMP amplicon was generated from 2 μL of 5 fM or 0 fM SARS-CoV-2 N-gene IVT RNA by amplifying at 62° C. for 20 minutes. Visualization of the Cas12 detection reaction was achieved using a FAM-biotin reporter molecule and lateral flow strips designed to capture labeled nucleic acids, as shown in FIG. 108A. Uncleaved reporter molecules are captured at the first detection line (control line), whereas indiscriminate Cas12 cleavage activity generates a signal at the second detection line (test line). To compare the signal generated by Cas12 when using fluorescence or lateral flow, RT-LAMP was performed using 5 fM or 0 fM IVT template using N gene primers and monitored the performance of the Cas12 readout on identical amplicons using a fluorescent plate reader and by lateral flow at 0, 2.5, 5, and 10 minutes. The Cas12 fluorescent signal was detectable in <1 minute, and a visual signal by lateral flow was achieved within 5 minutes. FIG. 112A shows the results of a DETECTR assay using LbCas12a to determine the effect of reaction time for a sample containing either 0 fM SARS-CoV-2 RNA or 5 fM SARS-CoV-2 RNA. Fluorescence signal of LbCas12a (SEQ ID NO: 27) detection assay on RT-LAMP amplicon for SARS-CoV-2 N-gene saturated within 10 minutes. RT-LAMP amplicon was generated from 2 μL of 5 fM or 0 fM SARS-CoV-2 N-gene IVT RNA by amplifying at 62° C. for 20 minutes.

FIG. 108C shows lateral flow test strips assaying samples corresponding to the samples assayed by DETECTR in FIG. 108B. Bands corresponding to control (C) or test (T) are shown for samples containing either 0 fM SARS-CoV-2 RNA (“−”) or 5 fM SARS-CoV-2 RNA (“+”) as a function of reaction time. LbCas12a on the same RT-LAMP amplicon produced visible signal through lateral flow assay within 5 minutes. FIG. 112B shows lateral flow test strips assaying samples corresponding to the samples assayed by DETECTR in FIG. 112A. Bands corresponding to control (C) or test (T) are shown for samples containing either 0 fM SARS-CoV-2 RNA (“−”) or 5 fM SARS-CoV-2 RNA (“+”) as a function of reaction time. LbCas12a (SEQ ID NO: 27) on the same RT-LAMP amplicon as shown in FIG. 112A produced visible signal through lateral flow assay within 5 minutes.

### Example 49

### Detection of SARS-CoV-2 in Patient Samples using a DETECTR Assay

This example describes detection of SARS-CoV-2 in patient samples using a DETECTR assay. Extracted RNA from nasal swab samples collected from six patients with documented SARS-CoV-2 infection, nasal swab samples from 15 patients with other influenza or coronavirus infections, and nasal swab samples from five healthy donors were tested. RNA extracts from patients with influenza (n=4) and other human coronavirus infections (common human seasonal coronavirus infections (OC34, HKU1, 229E and NL63, n=7)) were compared to in vitro transcribed SARS-CoV-2 target RNA spiked into nasal swab matrix in UTM and RNA extracted from nasal swabs from 2 SARS-CoV-2 infected patients. Samples were detected using SARS-CoV-2 DETECTR assay with fluorescence and lateral flow strip readouts FIG. 109 shows a table comparing the SARS-CoV-2 DETECTR assay with RT-LAMP of the present disclosure to the SARS-CoV-2 assay with a quantitative reverse transcription polymerase chain reaction (qRT-PCR) detection method. The N-gene target in the DETECTR RT-LAMP assay overlaps the N-gene N2 amplicon detected in the qRT-PCR assay. FIG. 108E shows patient sample DETECTR data. Clinical samples from 6 patients with COVID-19 infection (n=11, 5 replicates) and 12 patients infected with influenza or one of the 4 seasonal coronaviruses (HCoV-229E, HCoV-HKU1, HCoV-NL63, HCoV-OC43) (n=12) were analyzed using SARS-CoV-2 DETECTR (shaded boxes). Signal intensities from lateral flow strips were quantified using ImageJ and normalized to the highest value within the N gene, E gene or RNase P set, with a positive threshold at five standard deviations above background. Final determination of the SARS-CoV-2 test was based on the interpretation matrix in FIG. 107E. FluA denotes Influenza A, and FluB denotes Influenza B. HCoV denotes human coronavirus. FIG. 108F shows lateral flow test strips testing for SARS-CoV-2 in a patient with COVID-19 (positive for SARS-CoV-2, “patient 11”), a no target control sample lacking the target nucleic acid (“NTC”), and a positive control sample containing the target nucleic acid (“PC”). The E-gene was detected using a gRNA corresponding to SEQ ID NO: 325. The N-gene was detected using a gRNA corresponding to SEQ ID NO: 323. All three samples were tested for the presence of the SARS-CoV-2 N-gene, the SARS-CoV-2 E-gene, and RNase P. There was 100% concordance of the results of the Cas12 based assays with the CDC N1/N2 qRT-PCR assays, demonstrating the feasibility of using the DETECTR Cas12 based assays for diagnosing patients with SARS-CoV-2 infection.

SARS-CoV-2 was detected in 9 of the 11 patient swabs and did not cross react with the other respiratory viruses. The two negative swabs from COVID-19 patients were confirmed to be below the established limit of detection. FIG. 118 shows lateral flow DETECTR results on 10 COVID-19 infected patient samples and 12 patient samples for other viral respiratory infections. Ten samples from 6 patients (COVID19-1 to COVID19-5) with one nasopharyngeal swab (A) and one oropharyngeal swab (B) were tested for SARS-CoV-2 using two different genes, N2 and E as well as a sample input control, RNase P. Results were analyzed in accordance with the guidance provided in FIG. 119. FIG. 119 shows instructions for the interpretation of SARS-CoV-2 DETECTR lateral flow results. FIG. 120A-C show fluorescent DETECTR kinetic curves performed on 11 COVID-19 infected patient samples and 12 patient samples for other viral respiratory infections. Ten nasopharyngeal/oropharyngeal swab samples from 6 patients (COVID19-1 to COVID19-6) were tested for SARS-CoV-2 using two different genes, N2 and E as well as a sample input control, RNase P.

FIG. 120A shows samples tested using the standard amplification and detection conditions, 10 of the 12 COVID-19 positive patient samples resulted in robust fluorescence curves indicating presence of the SARS-CoV-2 E gene (20-minute amplification, signal within 10 min). No E gene signal was detected in the 12 other viral respiratory clinical samples.

FIG. 120B shows samples tested for the presence of the SARS-CoV-2 N gene using an extended amplification time to produce strong fluorescence curves (30-minute amplification, signal within 10 min) for 10 of the 12 COVID-19 positive patient samples. No N gene signal was detected in the 12 other viral respiratory clinical samples.

FIG. 120C shows graphs corresponding to the sample input control, RNase P.

Given the 100% concordance between lateral flow and fluorescence-based readouts shown in FIG. 120 and FIG. 121, a fluorescence-based readout was used to blindly test an additional 60 nasopharyngeal swab samples from patients with acute respiratory infection for SARS-CoV2 using our DETECTR assay. Of the 60 samples, 30 were positive for COVID-19 infection by qRT-PCR testing and 30 were negative for COVID-19 infection but either positive for another viral respiratory infection by respiratory virus panel (RVP) multiplex PCR testing or negative by all testing. The positive predictive agreement (PPA) and negative predictive agreement (NPA) of SARS-CoV-2 DETECTR relative to the CDC qRT-PCR assay were 95% and 100%, respectively, for detection of the coronavirus in 83 total respiratory swab samples.

FIG. 121 shows heatmaps of SARS-CoV-2 DETECTR assay results for clinical samples with the test interpretation indicated. Results of lateral flow SARS-CoV-2 DETECTR assay (top) quantified by ImageJ Gel Analyzer tools for SARS-CoV-2 DETECTR on 24 clinical samples (12 COVID-19 positive) show 98.6% (71/72 strips) agreement with the results of the fluorescent version of the assay (bottom). Both assays were run with 30-minute amplification, Cas12 reaction signal taken at 10 min. Presumptive positive indicated by (+) in orange (bottom, column 4).

FIG. 122 shows heatmaps of SARS-CoV-2 DETECTR assay results for clinical samples with the test interpretation indicated. The top plot shows result of fluorescent SARS-CoV-2 DETECTR assay on an additional 30 COVID-19 positive clinical samples (27 positive, 1 presumptive positive, 2 negative). Presumptive positive indicated by (+) in orange (top, column 9). The bottom plot shows result of fluorescent SARS-CoV-2 DETECTR assay on an additional 30 COVID-19 negative clinical samples (0 positive, 30 negative).

Relative to the CDC qRT-PCR protocol, the SARS-CoV-2 DETECTR assay was 90% sensitive and 100% specific for detection of the coronavirus in nasal swab samples, corresponding to positive and negative predictive values of 100% and 91.7%, respectively. FIG. 108G shows performance characteristics of the SARS-CoV-2 DETECTR assay. fM denotes femtomolar; NTC denotes no-template control; PPV denotes positive predictive value; NPV denotes negative predictive value. 83 clinical samples (41 COVID-19 positive, 42 negative) were evaluated using the fluorescent version of the SARS-CoV-2 DETECTR assay. One sample (COVID19-3) was omitted due to failing assay quality control. Positive and negative calls were based on criteria described in FIG. 107E. fM denotes femtomolar; NTC denotes no-template control; PPA denotes positive predictive agreement; NPA denotes negative predictive agreement.

SARS-CoV-2 DETECTR assay (RT-LAMP+Cas12a) was evaluated on IVT RNA products from SARS-CoV-2, SARS-CoV, bast-SL-CoVZC45, and clinical samples from common human coronaviruses. FIG. 113 shows the results of a DETECTR assay to determine the cross-reactivity of gRNAs for different human coronavirus strains. Samples containing in vitro transcribed RNA of the SARS-CoV-2 N-gene, the SARS-CoV N-gene, the bat-SL-CoVZC45 N-gene, the SARS-CoV-2 E-gene, the SARS-CoV E-gene, or the bat-SL-CoVZC45 E-gene, or clinical samples positive for CoV-HKU1, CoV-299E, CoV-OC43, or CoV-NL63 were tested. HeLa total RNA was tested as a positive control for RNase P, and a sample lacking a target nucleic acid (“NTC”) was tested as a negative control. The N-gene was detected using a gRNA corresponding to SEQ ID NO: 323. The E-gene was detected using a gRNA corresponding to SEQ ID NO: 325. RNase P was detected using a gRNA corresponding to SEQ ID NO: 330. The SARS-CoV-2 DETECTR assay was positive only from the in vitro transcribed SARS-CoV-2 spiked samples and nasal swab samples from SARS-CoV-2 infected patients, indicating that the DETECTR assay was specific for SARS-CoV-2. The N-gene was only detected in SARS-CoV-2, whereas the E-gene was detected only in SARS-CoV-2 and bat-SL-CoVZC45. SARS-CoV E-gene was not detected as the RT-LAMP primer set was not capable of amplifying the SARS-CoV E-gene, even though the E-gene gRNA was capable of detecting the SARS-CoV E-gene target site. RNase P was detected in common human coronaviruses because these samples are RNA extracted from clinical samples. Result are shown at 15 minutes of LbCas12a (SEQ ID NO: 27) detection assay signal on fluorescent plate reader. FIG. 113 shows the target on the x-axis, which from left to right are SARS-CoV-2 N-gene, SARS-Cov N-gene, bat-SL-CovZC45 N-gene, SARS-2 E-gene, SARS-CoV E-gene, Bat-SL-CoVZC45 E-gene, Cov-HKUI, Cov-299E, Cov-OC43, Cov-NL63, HeLa total RNA, and NTC. Within each target group are three bars, which from left to right show varying gRNA including N-gene, E-gene, and RNaseP. The y-axis shows raw fluorescence in arbitrary units (AU) ranging from 0 to 3000 in increments of 1000.

FIG. 115A-FIG. 115B show DETECTR kinetic curves on COVID-19 infected patient samples. Ten nasal swab samples from 5 patients (COVID19-1 to COVID19-10) were tested for SARS-CoV-2 using two different genes, N2 and E as well as a sample input control, RNase P. FIG. 115A shows using the standard amplification and detection conditions, 9 of the 10 patients resulted in robust fluorescence curves indicating presence of the SARS-CoV-2 E-gene (20 minute amplification, signal within 10 minutes). FIG. 115B shows the SARS-CoV-2 N-gene required extended amplification time to produce strong fluorescence curves (30 minute amplification, signal within 10 minutes) for 8 of the 10 patients. FIG. 115C shows that as a sample input control, RNase P was positive for 17 of the 22 total samples tested (20 minute amplification, signal within 10 minutes).

### Example 50

### Improved Detection of an RNase P POP7 Control Gene with Modified LAMP Primers and gRNA

This example describes improved detection of an RNase P POP7 control gene with modified LAMP primers and gRNA. Samples containing RNase P POP7 RNA were assayed using RT-LAMP and DETECTR reactions to assess the amplification and detection efficiency of primer sets and gRNAs directed to RNase P POP7. Samples containing either 0.16 ng/μL total RNA or 0 ng/μL total RNA were amplified by RT-LAMP with different primer sets at 60° C. for 60 minutes. FIG. 123 shows the time to result for RT-LAMP amplification of RNase P POP7 with different primer sets. Time to result was determined for samples amplified with primer sets 1-10. Primer set 1 corresponds to SEQ ID NO: 360-SEQ ID NO: 365, and primer set 9 corresponds to SEQ ID NO: 366-SEQ ID NO: 371. Primer set 9 showed improved time to result over primer set 1 and primer sets 2-8 and 10 for samples containing 0.16 ng/μL total RNA. Additionally, primer set 9 showed less non-specific amplification of samples without total RNA (0 ng/μL total RNA) than primer set 1 and primer sets 2, 3, 7, 8, and 10.

A DETECTR reaction was performed on the amplicons generated by RT-LAMP. Samples were detected using gRNAs corresponding to R779 (SEQ ID NO: 330), R780 (SEQ ID NO: 332), or R1965 (SEQ ID NO: 331). FIG. 124 shows raw fluorescence over time of a DETECTR reaction performed on RNase P POP7 amplified using RT-LAMP with primer set 1 or primer set 9 and detected with R779, R780, or R1965 gRNAs. The DETECTR reaction was carried out at 37° C. for 90 minutes. The amplicon generated by the set 1 primers were detected without background (dotted line) by R779. Clean detection was also seen by R1965 and R780 on amplicons generated by set 9. The results show that R1965 detects faster than R779 or R780. FIG. 124 shows two line graphs of DETECTR reactions. The graph on the left shows sett (original) and the graph on the right shows set9. Within each graph are three crRNA including R779, R780, and R1965. Solid lines indicate 0.16 ng/ul of total RNA and dashed lines indicate 0 ng/ul. The x-axis shows time in minutes ranging from 0 to 30 in increments of 10. The y-axis shows raw fluorescence in arbitrary units (AU) from 0 to 60000 in increments of 20000. In the left graph, R1965 shows the highest signal most quickly, followed by R779, and R1965 at a total RNA of 0 ng/ul. In the right graph, R1965 shows the highest signal most quickly followed by R780.

The limit of detection was then tested for RNase P POP7 amplified using RT-LAMP with primer set 1 (SEQ ID NO: 360-SEQ ID NO: 365) or primer set 9 (SEQ ID NO: 366-SEQ ID NO: 371) and detected with R779 gRNA (SEQ ID NO: 330) or R1965 gRNA (SEQ ID NO: 331). FIG. 125A shows the time to result of RNase P POP7 detection in samples containing 10-fold dilutions of total RNA amplified using RT-LAMP with primer set 1 or primer set 9. Amplification was carried out at 60° C. for 30 minutes. FIG. 125B shows a DETECTR reaction of the RNase P POP7 amplicons shown in FIG. 125A and detected using gRNA 779 (SEQ ID NO: 330) or gRNA 1965 (SEQ ID NO: 331). Samples amplified using primer set 1 were detected with gRNA 779 and samples amplified with primer set 9 were detected with gRNA 1965. The DETECTR reaction was carried out at 37° C. for 90 minutes. Primer set 9 showed improved time to limit of detection, as seen by a faster time to result at low RNA concentrations, compared to primer set 1. Additionally, primer set 9 showed improved speed and sensitivity in a DETECTR reaction when detected with gRNA 1965 as compared to samples amplified with primer set 1 and detected with gRNA 779. FIG. 125A shows a bar graph titled RT-LAMP RNase P POP7-LAMP. The x-axis shows concentration, which from left to right include 2 ng/ul, 0.2 ng/ul, 0.02 ng/ul, 0.002 ng/ul, 0.0002 ng/ul, 0.00002 ng/ul, 0.000002 ng/ul, and 0 ng/ul. The y-axis shows time to result in minutes ranging from 0 to 30 in increments of 10. Within each group on the x-axis are two bars indicating the primer set used, which from left to right are sett (original) and set9. FIG. 125B shows line graphs titled RNase P POP7 primer set comparison—DETECTR. The x-axis on each graph shows the time in minutes ranging from 0 to 30 in increments of 10. The y-axis on each graph shows the raw fluorescence in arbitrary units (AU) ranging from 0 to 50000 in increments of 10000. The graphs from left to right show decreasing concentrations of 2 ng/ul, 0.2 ng/ul, 0.02 ng/ul, 0.002 ng/ul, and 0 ng/ul. Within each graph are two lines which are varying crRNA including R779 and R1965. In the left most graph, while both crRNA eventually reach the same raw fluorescence value, the R1965 line reaches max fluorescence more quickly. In the second graph from the left, while both crRNA eventually reach the same raw fluorescence value, the R1965 line reaches max fluorescence more quickly. In the third graph from the left, while both crRNA eventually reach the same raw fluorescence value, the R1965 line reaches max fluorescence more quickly. In the fourth graph from the left, the R1965 line reaches the highest max fluorescence most quickly.

### Example 51

### Viral Lysis Buffer for Lysis and Amplification of a Coronavirus

This example describes a viral lysis buffer for lysis and amplification of a coronavirus. Nasal swab or saliva samples are collected from individuals suspected of having a coronavirus infection. Nasal swab and saliva samples are suspended in a viral lysis buffer formulated to lyse the viral capsids and release the viral genome. The viral lysis buffer is compatible with RT-LAMP amplification of the viral genome and DETECTR detection of a target nucleic acid, providing a one-step sample preparation solution for a coronavirus DETECTR reaction.

### Example 52

### Detection of a SNP Using a DETECTR Assay on a Microfluidic Cartridge

This example describes detection of a SNP using a DETECTR assay on a microfluidic cartridge. This assay was performed on a microfluidic cartridge shown in FIG. 126B. A cartridge manifold for configured to heat the cartridge was turned on. 5 μL of a sample from a blue-eyed individual was combined with 45 μL of a LAMP master mix solution containing the components for LAMP amplification of the sample. The sample was pre-mixed before being added to the cartridge. The pre-mixed sample was loaded into the cartridge in the amplification chamber, and the chamber was sealed with clear tape. 95 μL of blue eye RNP (G SNP) was loaded into the DETECTR chamber. The loaded chip was transferred onto the pre-heated manifold and sealed with clear tape.

The first heater of the manifold was set to 60° C., and the second heater was set to 37° C. The sample was incubated for 30 minutes at 60° C. After 30 minutes, a first pump in the manifold was initiated to pump the LAMP buffer with the sample through the cartridge. A second pump in the manifold was initiated to push 95 μL of the DETECTR solution into the detection chamber. The sample was incubated at 37° C. for 30 minutes. Fluorescence was visualized using a black box fluorescence detector.

A control assay was performed in microcentrifuge tubes using a heating block. In a first tube, 5 μL of a sample from a blue-eyed individual was combined with 45 μL of a LAMP master mix solution. In a second tube, 5 μL of a sample from a brown-eyed individual was combined with 45 μL of a LAMP master mix solution. Samples were incubated for 30 minutes at 60° C. in a mini dry bath. 5 μL of each amplified sample was transferred to 95 μL of a 1× RNP solution for detection of A and G SNPs. The reactions were transferred to a 37° C. heat block.

### Example 53

### Amplification and Detection of a SNP in a Microfluidic Cartridge

This example describes amplification and detection of a SNP in a microfluidic cartridge. These assays were performed in the microfluidic cartridge illustrated in FIG. 128B. The following solutions were prepared: LAMP master mix (1× IsoAmp Buffer (NEB), 4.5 mM MgSO₄, 1.4 mM dNTPs, 1:5 Bst 2.0 (NEB), 1× primer master mix, and 1:10 target DNA), and CRISPR complex (1× MBuffer3, 40 nM crRNA, and 40 nM Cas12 variant (SEQ ID NO: 37); 1 μM reporter substrate was added after incubated at 37° C.).

PMMA layers of the cartridge were cleaned by immersion in RNAse Zap for 20 minutes and washing of remnants of the cleaning solution by washing twice in nuclease free water. The cartridge was dried using a stream of nitrogen. The layers of the cartridge were assembled. The top half of the CRISPR reaction workflow was blocked with high sol epoxy and dried for 20 minutes until clear. 80 μL of LAMP master mix was pre-mixed in a microcentrifuge tube with 10 μL of primer mix and 10 μL of pure DNA extract. The solution was mixed by pipetting up and down. 70 μL of this solution was loaded into the amplification chamber of the cartridge using a pipette. The chamber was sealed using a small rectangular piece of PCR adhesive (Biorad, MSB-1001).

The cartridge was placed into a heating manifold, and the aluminum block was heated to an on-chip temperature of 60° C. The sample was incubated at 60° C. for 30 minutes to amplify the sample using LAMP. 100 μL of CRISPR reagent containing a blue-eye gRNA was added to the lower DETECTR chamber. The top and bottom chambers were sealed with small rectangular pieces of PCR adhesive. The CRISPR reagents were mixed with 5 μL of the amplified sample by actuating a valve in the cartridge. The manifold was covered with a shroud of 3D printed APS to block light. The aluminum block was heated to an on-chip temperature of 37° C. The CRISPR reaction was incubated for 30 minutes at 37° C. The resulting fluorescence was observed by eye.

The assay was repeated as described above using the cartridge illustrated in FIG. 128C, except that the top half was not sealed with epoxy. In both assays, the fluorescence corresponding to a positive result was observable by eye. Illumination of the cartridges in the manifold from the top of the cartridge resulted in uneven illumination of the detection chambers.

### Example 54

### Amplification and Detection of a SNP in a Revised Microfluidic Cartridge

This example describes amplification and detection of a SNP in a revised microfluidic cartridge. This assay was performed on a microfluidic cartridge illustrated in FIG. 129A. LAMP master mix and CRISPR complex solutions were prepared as described in EXAMPLE 53. PMMA layers of the cartridge were cleaned by immersion in RNAse Zap for 20 minutes and washing of remnants of the cleaning solution by washing twice in nuclease free water. The cartridge was dried using a stream of nitrogen. The layers of the cartridge were assembled.

40 μL of LAMP master mix was pre-mixed in a microcentrifuge tube with 5 μL of primer mix and 5 μL of pure DNA extract. The solution was mixed by pipetting up and down. 50 μL of this solution was loaded into the amplification chamber of the cartridge using a pipette. The chamber was sealed using a small rectangular piece of PCR adhesive (Biorad, MSB-1001). 95 μL of the CRISPR reagent solution containing a Cas12 variant (SEQ ID NO: 37) and a gRNA directed to a brown-eye SNP was added to the lower DETECTR chamber, and 95 μL of a negative reagent solution (5× MBuffer3) was added to the upper DETECTR chamber. The chamber was sealed using a small rectangular piece of PCR adhesive (Biorad, MSB-1001).

The cartridge was assembled on a heating manifold, and the aluminum block was heated to an on-chip temperature of 60° C. at the amplification chamber. Heating was initiated 2 minutes prior to beginning the assay. Amplification was performed at 60° C. for 30 minutes. The valve of the cartridge was actuated to mix the CRISPR reagent with 5 μL of the amplified sample. The manifold heater of the detection chamber was heated to 37° C. without pre-heating. The DETECTR reaction was performed at 37° C. for 30 minutes, and the resulting fluorescence was observed by eye. The chambers were imaged by illuminating with either an LED from a mini PCR kit or an LED from ThorLabs.

The assay was repeated on a new cartridge of the same design with the following modifications: the CRISPR reagents were not preloaded into the device, because the heater was still warm from the previous run, and the amplification and detection steps were run for 15 minutes instead of 30 minutes.

A third assay was performed on a microfluidic cartridge illustrated in FIG. 129B. The amplification chamber was loaded with 50 μL of nuclease free water and the chamber was sealed with a small piece of PCR adhesive. 50 μL of 1 μM ATTO-488 dye and 45 μL of nuclease free water were loaded into the lower CRISPR chamber, and 95 μL of nuclease free water was loaded into the upper CRISPR chamber. Both chambers were sealed with a small piece of PCR adhesive. The cartridge was assembled on a heating manifold, as shown in FIG. 137B. Samples were incubated for 10 seconds in the amplification chamber. The first pump was run for 3 seconds to drive 5μL of fluid out of the amplification chamber and into the CRISPR chamber (also referred to as the detection chamber). The second pump was run for 5 seconds to drive detection reagents into the CRISPER chamber. The samples were incubated in the CRISPR chamber for 10 seconds before illuminating with an LED. The assay was repeated with the following parameters: 30-minute incubation in the amplification chamber, pump 1 run for 1 second, pump 2 run for 20 seconds, and 15-minute incubation in the CRISPR chamber before illuminating with an LED. The longer pump times improved fluid transfer between chambers.

### Example 55

### Use of a Microfluidic Device for a DETECTR Reaction

This example describes use of a microfluidic device for a DETECTR reaction. A microfluidic cartridge as illustrated in any of FIG. 126A, FIG. 126B, FIG. 127A, FIG. 127B, FIG. 128A, FIG. 128B, FIG. 128C, FIG. 128D, FIG. 129A, FIG. 129B, FIG. 129C, or FIG. 129D is loaded with amplification reagents and DETECTR reagents. 50 μL of amplification reagent is added to the amplification chamber, and 95 μL of DETECTR reagent is added to DETECTR chamber. The wells of the cartridge are sealed. The cartridge is loaded into a heating manifold as illustrated in any of FIG. 136A, FIG. 136B, FIG. 137B, FIG. 137C, or FIG. 138A-B. The cartridge is inserted in a specific orientation. Screws are tightened to hold the cartridge in place. Openings are sealed with clear qPCR tape cut to size to create an air-tight seal. A thermocouple is inserted into the amplification chamber to record temperatures. The solenoid, shown in FIG. 130A, is energized to close the valve. Indicator LED lights turn on. Two heaters, set to 60° C. and 37° C., are turned on. The sample is incubated at 60° C. for 30 minutes in the amplification chamber. The solenoid is de-energized to open the valve. Pump 1 is activated for 15 seconds to move fluid from the amplification chamber to the DETECTR reaction chambers. After 15 seconds, pump 2 is activated for 15 seconds to move fluid from the DETECTR reagent reservoirs to the DETECTR reaction chambers. The sample is incubated in the DETECTR reaction chambers for 30 minutes at 37° C. The indicator light turns off. The LED is turned on and fluorescence is measured by image, visual assessment, or photodiode detection.

At the end of the 30-minute 60° C. LAMP incubation, the solenoid valve opens and the peristaltic pump #1 engages at 100% PWM for 10 seconds. The LAMP buffer is pumped through the valve to the intersection of the serpentine channels leading to the DETECTR reaction chambers and the straight channels leading to the DETECTR reagent reservoirs. The serpentine channel leading to the DETECTR reaction chambers has a larger cross-sectional area than the channel leading to the DETECTR reagent reservoirs. This is intended to reduce the fluidic resistance in the serpentine channels and direct all of the buffer towards the DETECTR reaction chambers. However, throughout this study (testing 23+ chips), the buffer has split both ways nearly every time, with approximately half the buffer volume going the wrong way. In the next fluidic step, the solenoid valve closes and DETECTR reagent is pumped towards the DETECTR reaction chambers, collecting the LAMP product along the way. This provides some mixing as both buffers travel through the serpentine channels simultaneously, but this process also creates bubbles that can get carried to the DETECTR chamber.

To prevent bubbles from interfering with fluorescence measurements during DETECTR, a larger volume of buffer is loaded into the reservoirs than the reaction chambers can fit and use a longer pumping time than necessary. This ensures that the chambers are completely filled with reagent and all bubbles have been popped. The DETECTR reaction chambers have a 70 μL volume, and 25 μL LAMP plus 95 μL DETECTR reagent are delivered into each chamber. The second fluidic step (DETECTR reagent to the DETECTR reaction chambers) takes about 20-30 seconds to deliver all the buffer, but this step is run for 45 seconds. This results in completely full DETECTR reaction chambers, with the excess reagents backed up in the serpentine channels. In addition to bubbles, if the DETECTR reaction chambers are not completely filled, condensation forms on the top of the chamber during the 37° C. incubation, which also interferes with fluorescence measurements taken from above.

### Example 56

### Thermal Testing of a Microfluidic Device for a DETECTR Reaction

This example describes thermal testing of a microfluidic device for a DETECTR reaction. The thermal performance of a heating manifold was tested by measuring the time to temperature and the accuracy of heating to the setpoints with thermocouples submerged within the buffer. Under standard assay temperature setpoints (60° C. LAMP/37° C. DETECTR), the LAMP buffer heats to 60° C. in 8.5 minutes, but the DETECTR buffer reaches a maximum temperature of 34° C. at around 21 minutes. This is somewhat counterintuitive, since it takes longer to hit a lower temperature (and the DETECTR buffer does not reach the setpoint temperature). To hit a specific temperature, the heater controller varies the amount of time it spends in the on state. This state switching is quantified by the pulsed width modulation (PWM) value, the percentage of a given unit of time it spends in the on state. The heater controller also samples the temperature of the heater for feedback on the difference between the current temperature and the setpoint temperature. The larger the difference between those two values, the higher the resulting PWM value will be. As the heater temperature approaches the setpoint, the PWM value drops to slow the rate of change and avoid overshooting the setpoint temperature. The difference between the room temperature heater and the LAMP setpoint is about 35° C., while the difference between the DETECTR heater and its setpoint is about 12° C. The LAMP incubation heats with maximum PWM values around 20%, and the DETECTR incubation heats with maximum PWM values around 12%. Our current setup is designed with a larger emphasis on accuracy and not overshooting the setpoint temperature than heating the buffer to assay temperature quickly.

Specific PWM values can be used to heat to our setpoint temperatures faster. However, this is a manual process and can result in overshooting the target temperatures and damaging the manifold prototype and melting the microfluidic chip. With the LAMP heater PWM value set to 100%, the LAMP buffer (measured by thermocouple) heats to 60° C. in 90 seconds, but the heater temperature hits 100° C. With the DETECTR heater PWM set to 100%, the DETECTR buffer heats to 37° C. in 60 seconds, and the heater hits 80° C. Turning the heater off when the DETECTR buffer hits 37° C. results in a maximum buffer temperature of around 60° C. the temperature of the DETECTR side of the chip rises during the 30-minute 60° C. LAMP incubation so that it is higher than room temperature. It varies from time to time, but it is usually between 25-29° C. by the beginning of the DETECTR side.

FIG. 139A, FIG. 139B, FIG. 140A, and FIG. 140B show thermal testing summaries for an amplification chamber heated to 60° C. (FIG. 139A and FIG. 140A) or a DETECTR chamber heated to 37° C. (FIG. 139B and FIG. 140B). FIG. 140A shows a graph titled BOBv2 LAMP Temperature vs Time (61° C. setpoint). The x-axis shows time in seconds from 0 to 1800 in increments of 200. The y-axis shows temperature in ° C. ranging from 20 to 65 in increments of 5. The graph includes two lines representing heater and buffer. While both the heater and buffer lines reach the same temperature eventually, the heater line achieves the max temperature more quickly. FIG. 140B shows a graph titled BOBv2 LAMP Temperature vs Time (40° C. setpoint). The x-axis shows time in seconds from 0 to 1800 in increments of 200. The y-axis shows temperature in ° C. ranging from 25 to 43 in increments of 2. The graph includes two lines representing heater and buffer. The heater line reaches a higher temperature more quickly.

### Example 57

### Detection of a HERC2 SNP Using a Microfluidic Cartridge

This example describes detection of a HERC2 SNP using a microfluidic cartridge. A primer mix containing 2 μM F3 primer, 2 μM B3 primer, 16 μM FIP primer, 16 μM BIP primer, 8 μM LF primer, and 8 μM LB primer in nuclease free water was prepared. A complexing reaction containing 1× MBuffer3, 40 nM crRNA, and 50 nM Cas12 variant (SEQ ID NO: 37) was prepared. 40 nM reporter substrate was added after incubating at 37° C. for 30 minutes. A LAMP mix containing 1× IsoAmp Buffer, 4.5 mM MgSO₄, dNTPs, and 1× primer mix was prepared. DETECTR reagents were loaded into a microfluidic cartridge and wells were sealed with PCR tape. LAMP mix was mixed with primers and loaded into the cartridge. The narrow end of the Chip Shop tank was covered with parafilm and inserted into the luer connection above the LAMP reaction chamber. The Chip Shop tank was loaded with 200 μL of 20 mM NaOH. The cartridge was inserted into the heating manifold and screws were tightened. A buccal swab was added to the tank, gently agitated, and incubated for 2 minutes. A Drummond micropipette was used to deliver 10 μL of lysed sample through parafilm into LAMP reaction chamber. The tank was removed and the chamber was sealed with qPCR tape cut to size.

FIG. 141A shows the DETECTR results run on a plate reader at a gain of 100, using the LAMP product from the microfluidic cartridge as an input. The samples were run in duplicate with a single non-template control (NTC). 19 μL of the DETECTR master mix (the same mixture used on the device) was pipetted into wells of a 384-well plate and 1 μL of LAMP amplicon was added. For one sample, 10 μL of amplicon was inadvertently added; that sample is represented by “10 μL target”. Because the donor is homozygous for the A-SNP, guide R570 was expected to generate a faster signal than R571. A slight difference was observed between the two samples. FIG. 141A shows a line graph with the x-axis showing time in minutes ranging from 0 to 30 in increments of 10 and the y-axis shows raw fluorescence in arbitrary units (AU) ranging from 0 to 60000 in increments of 20000. The bottom two flat lines are R570 NTC and R571 NTC. The lines achieving high signal rapidly include, from left to right, R570 10 ul, R 570 1 ul, and R571 1 ul.

FIG. 141B shows three LAMP products run on a plate reader using samples from a microfluidic chip. The LAMP reactions are numbered in the order that the chips were run (LAMP_1 was run first, etc.). The donor was homozygous for SNP A and, accordingly, crRNA 570 comes up first. The ATTO 488 was used as a fluorescence standard. These measurements were taken on a plate reader at a gain of 60. Results of the three LAMP reactions were clustered close together, which indicated good run-to-run reproducibility for amplification on the microfluidic cartridge and heating manifold. Each LAMP reaction was run in triplicate with each crRNA, generating the error ranges visible in the graph. FIG. 141B shows a line graph with the x-axis showing time in minutes ranging from 0 to 30 in increments of 10 and the y-axis shows raw fluorescence in arbitrary units (AU) ranging from 0 to 8000 in increments of 2000. The flat lines near the bottom of the graph are 10 nM ATT0488 None and NTC. The flat dashed line near 6000 AU is 100 nM ATT0488 None. The lines achieving high signal rapidly include, from left to right approximately, LAMP_1 R570 and LAMP_3 R570, LAMP_2 R570, LAMP_3 R571, LAMP_1 R571, and LAMP_2 R571.

Another assay was performed. Solutions were prepared as described above, and samples were run on a microfluidic cartridge shown in FIG. 129A with addition of a luer connector on top of the amplification chamber. A buccal swab sample was prepared as described above. The cartridge was loaded, and the assay was run with the following settings: 30 minutes amplification, 10 seconds Pump 1, 40 seconds Pump 2, 30 minutes DETECTR. Samples were measured on a plate reader. FIG. 142A an image of the microfluidic cartridge after the assay. The bluer appearance of the right well compared to the green appearance of the left well is likely due to the bubbles in the right well diffusing the input blue light. FIG. 142B shows results of a DETECTR reaction measured on a plate reader after 30 minutes of LAMP amplification. The bubbles in the one reaction chamber interfered with the signal from the ESE log, so the quantitative measurements shouldn't be trusted. However, the 10 minute and 20-minute timepoints had similar signals. Furthermore, both wells appeared visually bright when the LEDs turned on after 30 minutes of DETECTR. The DETECTR results on the plate reader showed that after 30 minutes the signal was high for both SNPs. FIG. 142B shows line graphs from left to right titled R570, R571, and None. The x-axis on each graph shows time in seconds ranging from 0 to 80 in increments of 20 and the y-axis on each graph shows raw fluorescence in arbitrary units (AU) ranging from 0 to 60000 in increments of 20000. In the leftmost graph, the NTC line is flat at the bottom, while the extracted DNA line achieves high fluorescence signal rapidly. In the middle graph, the NTC line is flat at the bottom, while the extracted DNA line achieves high fluorescence signal rapidly. In the right graph, the 10 nM ATTO line is flat at the bottom, the 10 nM ATTO line is flat near the middle, and the 100 nM ATTO line is flat at the top.

### Example 58

### Detection of a Coronavirus Using a Microfluidic Cartridge

This example describes detection of a coronavirus using a microfluidic cartridge. A complexing reaction containing 1× MBuffer3, 40 nM crRNA, and 50 nM Cas12 variant (SEQ ID NO: 37) was prepared. 40 nM reporter substrate was added after incubating at 37° C. for 30 minutes. 95 μL DETECTR reagents were loaded into each DETECTR reagent well and sealed with qPCR tape. A tube of N Gene LAMP master mix (537 μL) was mixed with 32 μL of 100 mM MgSO₄ and 40 μL of mixture was loaded into a cartridge. 10 μL of Twist SARS-Cov-2 standard was added at various copies/μL or 1× TE as a negative control to LAMP reaction chamber. The cartridge was inserted into the manifold and tightened. The LAMP reaction chamber was sealed with qPCR tape. Temperatures were set (62° C. LAMP, 40° C. DETECTR (to account for thermal offset)) and automated workflow was initiated. A 3D-printed optical cover was placed on the cartridge to minimize optical noise. DETECTR measurements were taken at 0 min, 2 min, 5 min, 10 min, 20 min, and 30 min. The copy number of RNA in the LAMP reaction was varied in order to estimate the lower limit of detection in the device.

FIG. 143A, FIG. 143B, FIG. 143C, and FIG. 143D show results of the coronavirus DETECTR reaction. The two reaction chambers with 10 copies input to LAMP resulted in a rapidly increasing DETECTR signal. All NTCs were negative. With 10 copies input into LAMP, the DETECTR signal gradually increased over the course of the reaction, as shown in the photodiode measurements below in FIG. 143C. The negative controls in FIG. 143D indicated an absence of contamination.

The assay was repeated. FIG. 144A, FIG. 144B, FIG. 144C, and FIG. 144D show the results of the repeated coronavirus DETECTR reaction.

### Example 59

### Turnaround Time of an Influenza B DETECTR Assy in a Microfluidic Cartridge

This example describes the turnaround time of an influenza B DETECTR assay in a microfluidic cartridge. A primer mix containing 2 μM F3 primer, 2 μM B3 primer, 16 μM FIP primer, 16 μM BIP primer, 8 μM LF primer, and 8 μM LB primer in nuclease free water was prepared. A complexing reaction containing 1× MBuffer3, 40 nM crRNA, and 50 nM Cas12 variant (SEQ ID NO: 37) was prepared. 40 nM reporter substrate was added after incubating at 37° C. for 30 minutes. 95 μL DETECTR reagents were loaded into each DETECTR reagent well and sealed with qPCR tape. 40 μL of LAMP mixture was added to the cartridge. 2 μL of 1 pM IBV target was added to 198 μL of viral lysis buffer and loaded into a Chip Shop tank. A Drummond micropipette was used to deliver 10 μL of lysed sample through parafilm into LAMP reaction chamber. The tank was removed and the chamber was sealed.

FIG. 145A, FIG. 145B, FIG. 146A, FIG. 146B, and FIG. 146C show the photodiode measurements for an influenza B DETECTR reaction in a microfluidic cartridge. 10 minutes of amplification time resulted in an increase in signal above the background (this was observed visually as well). 5 minutes of amplification time did not result in a visible increase in signal. FIG. 145A shows line graphs titled Aggregated DETECTR signals: IBV LAMPrey Time point Testing on the detection manifold. The x-axis shows time in minutes ranging from 0 to 25 in increments of 5. The y-axis shows raw fluorescence ranging from 0 to 0.5 in increments of 0.1. The 3 lines near the middle are 15 min LAMP, 5 min LAMP, and NTC with the topmost line of the 3 liens being 15 min lamp. The topmost line in the graph is 10 min LAMP. FIG. 145B shows line graphs titled DETECTR Signal: 15 min IBV LAMP. The x-axis shows time in minutes ranging from 0 to 30 in increments of 10. The y-axis shows raw fluorescence ranging from 0 to 0.5 in increments of 0.1. The two lines near the middle are Channel 1 and Channel 2, with the Channel 1 line being higher.

### Example 60

### A DETECTR Assay Utilizing Reagents Stored in Glass Capillaries

This example describes the use of glass capillaries in a DETECTR reaction. Glass capillaries can enable fluid flow by passive capillary action, thereby obviating the need for power-driven flow (e.g., with mechanical pumps). Glass capillaries are also capable of long-term, stable reagent storage.

The reagents required for a DETECTR reaction are provided in inside of the capillaries in dry form. Hydrating the capillaries solubilizes the reagents, and allows them to be eluted from the capillaries into a collector compartment or container. CRISPR-Cas complexes may be stored and recollected in this fashion without loss of activity.

DETECTR reagent mixes were prepared by pre-complexing a guide nucleic acid of SEQ ID NO: 374 with 5 μM of a programmable nuclease with SEQ ID NO: 37 in 5× MBuffer2 (20 mM Tris HCl, pH8, 100 mM NaCl, 5 mM MgCl2, 1 mM DTT, 5% glycerol, 50 ug/mL Heparin). 23.5 mm capillaries with 20 μl volume capacities were loaded with 0.5 μl droplets of a reagent mix and then dried at room temperature overnight. After 212 days, the capillaries were rehydrated with 20 μl aliquots of 5× MBuffer2 containing either 0 μM or 0.170 μM ssDNA substrates with fluorescent reporters and either 0 μM, 0.1 μM, or 1 μM target nucleic acid. The sequences of the guide nucleic acid, ssDNA substrates, and target nucleic acid are provided in TABLE 24 below. Following 2 minutes of room temperature rehydration, the contents of each capillary were expunged into separate wells on a 384-well plate. The wells were incubated at 37° C. for 90 minutes, during which time a fluorescence readout was monitored from each well.

TABLE 24

| Species | SEQ ID NO | Sequence |
|---|---|---|
| Guide nucleic | SEQ ID NO: | GGCUGGCCAAACUGCUGGGU |
| acid (R435) | 374 | |
| Reporter | SEQ ID NO: 9 | /56-FAM/TTATTATT/3IABkFQ/ |
| nucleic acid | | |
| rep01 | | |
| Target nucleic | SEQ ID NO: | TGGTCCCCGCCACCCCCCACCCCCACTTTGCAGA |
| acid | 375 | TAAACCACATGCAGGAAGGTCAGCCTGGCAAGT |
| | | CCAGTAAGTTCAAGCCCAGGTCTCAACTGGGCA |
| | | GCAGAGCTCCTGCTCTTCTTTGTCCTCATATACG |
| | | AGCACCTCTGGACTTAAAACTTGAGGAACTGGA |
| | | TGGAGAAAAGTTAATGGTCAGCAGCGGGTTACA |
| | | TCTTCTTTCATGCGCCTTTCCATTCTTTGGATCAG |
| | | TAGTCACTAACGTTCGCCAGCCATAAGTCCTCG |
| | | ACGTGGAGAGGCTCAGAGCCTGGCATGAACATG |
| | | ACCCTGAATTCGGATGCAGAGCTTCTTCCCATGA |
| | | TGATCTGTCCCTCACAGCAGGGTCTTCTCTGTTT |
| | | CAGGGCATGAACTACTTGGAGGACCGTCGCTTG |
| | | GTGCACCGCGACCTGGCAGCCAGGAACGTACTG |
| | | GTGAAAACACCGCAGCATGTCAAGATCACAGAT |
| | | TTTGGGCTGGCCAAACTGCTGGGTGCGGAAGAG |
| | | AAAGAATACCATGCAGAAGGAGGCAAAGTAAG |
| | | GAGGTGGCTTTAGGTCAGCCAGCATTTTCCTGAC |
| | | ACCAGGGACCAGGCTGCCTTCCCACTAGCTGTA |
| | | TTGTTTAACACATGCAGGGGAGGATGCTCTCCA |
| | | GACATTCTGGGTGAGCTCGCAGCAGCTGCTGCT |
| | | GGCAGCTGGGTCCAGCCAGGGTCTCCTGGTAGT |
| | | GTGAGCCAGAGCTGCTTTGGGAACAGTACTTGC |
| | | TGGGACAGTGAATGAGGATGTTATCCCCAGGTG |
| | | ATCATTAGCAAATGTTAGGTTTCAGTCTCTCCCT |
| | | GCAGGATATATAAGTCCCCTTCAATAGCGCAAT |
| | | TGGGAAAGGTCACAGCTGCCTTGGTGGTCCACT |
| | | GCTGTCAAGGACACCTAAGGAACAGGAAAGGC |
| | | CCCATGCGGACCCGAGCTCCCAGGGCTGTCTGT |
| | | GGCTCGTGGCTGGGACAGGCAGCAATGGAGTCC |
| | | TTCTCTCCCTTCACTGGCTCGGTTTCT |

The results of the DETECTR experiment are shown in FIG. 147. Fluorescence increases were detected for the reactions with 0.17 μM reporter nucleic acid and either 0.1 μM and 1.0 μM target nucleic acid. The results show that the pre-complexed guide nucleic acid-programmable nuclease complexes maintained catalytic activity after air-drying and long-term storage. Rehydration is rapid, making intracapillary desiccation or lyophilization an ideal storage method.

### Example 61

### Spin-Columns for Sequential Amplification and CRISPR Reactions

This example describes a device that is designed to mix reagents for a nucleic acid amplification reaction and a CRISPR reaction. Amplification reactions and CRISPR reactions often require separate buffers and conditions. Thus, performing sequential amplification and CRISPR reactions on a single sample can require exposing the sample to the surrounding environment. This example describes a multicompartment spin-column that can move a sample through separate compartments containing different reagents while remaining sealed to reduce contamination.

The spin-column can have the structure illustrated in FIG. 148 panel A. This spin column has a top compartment 101 that can be loaded with CRISPR reagents and a bottom compartment 102 that can be loaded with isothermal amplification reagents. A single cap 103 is capable of sealing both compartments from the exterior environment. The top compartment is isolated from the bottom compartment by a weakly permeable material 104 such as a membrane or filter. Downwards force or pressure within the top compartment can be used to move reagents through the weakly permeable material and into the bottom compartment. This can be achieved by centrifuging the spin-column, compressing the cap, or selectively heating the top compat linent. The top compartment may be removable from the bottom compartment. For example, the top compartment can be a tube that fits within the bottom compartment.

The spin-column can be used in the method illustrated in FIG. 148. As is shown in panel A, the top compartment is loaded with CRISPR reagents and the bottom compartment can is loaded with amplification reagents and the sample. Both compartments are sealed from the external environment by closing the cap at the top of the spin-column. The sample can then be subjected to an amplification reaction, as shown in panel B. During this phase, the spin-column may be incubated at a temperature suitable for the amplification reaction (e.g., to between 37 and 65° C.). Next, as is shown in panel C, the CRISPR reagents are drawn from the top compartment into the bottom compartment through the weakly permeable material by centrifugation. In panel D, the spin-column can then be incubated at a second temperature suitable for the CRISPR reaction. Optionally, a signal can be detected from the sample through the transparent spin-column material (panel E, e.g., if the CRISPR reaction produces a fluorescence signal).

### Example 62

### Electrochemically Detectable Reporter Molecules for DETECTR Reactions

This example describes reporter molecules with electrochemically detectable moieties for use in DETECTR reactions. The reporter molecules are ssDNA containing modified thymine nucleobases conjugated to ferrocene moieties and a fluorescent moiety (e.g., fluorescein) conjugated to the 5′ end via a phosphodiester linkage. Reporter molecules may be biotinylated at the 3′ end. The sequences of two ssDNA reporter molecules are 5′-YXXTTATTXX-3′ (SEQ ID NO: 391) and 5′-YXXTTATTATTXXZ-3′ (SEQ ID NO: 392), wherein X is ferrocene labeled thymidine (FIG. 149 panel A), Y is 6-carboxyfluoroscein (FIG. 149 panel B), and Z is a 3′ biotin moiety (FIG. 149 panel C).

In a DETECTR reaction, the reporter molecules may undergo transcollateral cleavage from a programmable nuclease (e.g., a Cas12 variant having a sequence of SEQ ID NO: 37). Reporter molecule cleavage mobilizes electrochemically detectable, ferrocene containing ssDNA subunits. Ferrocene has a relatively high oxidation potential, and thus can be potentiometrically detected against a background of low oxidation potential biomolecules. The magnitude of the electrochemical signal increases with cleavage of the reporter molecules. In contrast, the intensity of the fluorescence signal from the reporter molecules is invariant to the degree of transcollateral cleavage. Thus, a fluorescence readout can be used to calibrate the electrochemical measurements by quantifying the total concentration of reporter molecules present, and the combination of electrochemical and fluorescence measurements can be used to determine the fraction of reporter molecules which have been cleaved. The biotin serves as a capture moiety for the reporter or fragments of the reporter (e.g., with streptavidin).

The assays were performed with a 5′-YXXTTATTXX-3′ (SEQ ID NO: 391) reporter oligonucleotide, a programmable nuclease targeting HERC2, and a HERC2 target nucleic acid. Detection was performed with a DropSens p.STAT ECL instrument and DropSens screen-printed carbon electrodes. Aliquots of the DETECTR reaction were collected at multiple time points after its initiation.

FIG. 176 shows the results of a DETECTR reaction measured with square wave voltammetry. The reactions utilized 50 fM target nucleic acid and 2.4 μM reporter nucleic acid. As can be seen in FIG. 176, the signal intensities of the oxidation (panel A) and reduction (panel B) curves were greater for the sample collected 33 minutes after initiation of the DETECTR reaction than for the sample collected immediately following initiation of the DETECTR reaction. Error bars represent standard deviation of two measurements of the same solution, using three traces from each measurement.

FIG. 177 shows the results of DETECTR reactions measured with cyclic voltammetry. The reactions utilized 24 μM reporter nucleic acid and 500 pM target nucleic acid. As can be seen in FIG. 177, the signal increased between the 0 minute and 26 minute timepoints of the DETECTR reactions. Each trace shown in FIG. 177 is the average of three scans, of the same solution. Error bars represent the standard deviations.

### Example 63

### Device for Automating Sequential Amplification and CRISPR Reactions

This example describes a device capable of performing multiple amplification and CRISPR reactions on a sample. The device is capable of dividing a sample to perform multiple, distinct sequences of amplification and CRISPR reactions on different aliquots of a single input sample. The device houses a microfluidic chip containing multiple compartments for storing reagents and reacting the sample. The device is configured to detect signals produced from the CRISPR reactions (e.g., optical signals), and thus facilitates a plurality of measurements from a single sample input. A possible application of the device is to perform separate series of amplification and CRISPR reactions to assay a single biological sample for a large number of viruses.

A schematic for the microfluidic chip is depicted in FIG. 150. Upon insertion into the device, a biological sample will be transported a first compartment (V1), where the sample can be combined with a variety of solutions (e.g., lysis buffer) depending on the type of sample and the number and types of assays to be performed. In some assays, V1 will be preloaded with a dilution buffer prior to the sample being loaded. The device can move (e.g., via a pump) a controlled quantity of the sample (e.g., 5 μl) from the first compartment into a second compartment (V2), where it can be mixed with amplification reagents from P1. The device controls the temperature of V2 to facilitate an amplification reaction. The device transports portions of the amplification product from V2 to either V3 or V4, where the sample is mixed with reagents for CRISPR reactions. Sample from V3 and V4 can be transported to waste compartments (V5 and V6, respectively).

A depiction of the device is provided in FIG. 151. The device is configured to hold the microfluidic chip 101 below a sample inlet port 102. The inlet port contains a projection 103 (e.g., a pneumatically driven needle) that can pull a sample into a first compartment in the microfluidic chip 104. The microfluidic chip can be removed and replaced, and is held over temperature control elements 105 that modulate the temperature within compartments in the microfluidic chip. The device contains a diode array 106 configured to measure absorbance and fluorescence from multiple microfluidic chip compartments. The device utilizes batteries 107 as a power source.

### Example 64

### Flu DETECTR Reaction with Dual Amplification, Viral Lysis Buffer System

This example describes an assay for detecting flu viral nucleic acids. The assay is a combination of ambient temperature RT-LAMP amplification and guide nucleic acid driven, programmable nuclease-based detection. LAMP protocols often require strict operating temperatures that are unfeasible for implementation in devices that perform multiple types of reactions. For example, the high temperatures required for some amplification reactions can damage reagents for CRISPR reactions. This example discloses activators for LAMP amplification that are operable at a range of temperatures, including ambient temperatures, that are more suitable for implementation within a device. This example also provides viral lysis buffers containing the LAMP activators, enabling concurrent lysis and amplification upon input of a sample, such as a swab containing nucleic acids associated with the flu.

A variety of potential LAMP activators were tested for LAMP activating capacity and viral lysis buffer compatibility. LAMP activating capacity was evaluated by performing dual LAMP-DETECTR assays in the absence of individual LAMP activators. In these assays, LAMP was performed with three out of four of a buffering agent, an activator, dNTPs, and primer. The DETECTR reactions were performed on buccal swab samples with SEQ ID NO: 37 and the guide nucleic acid (targeting HERC2) given in TABLE 25 below. The DETECTR reactions were monitored by fluorescence over 90 minutes. A separate control assay was performed with all four reagents present during the LAMP amplification. As shown in FIG. 152, the LAMP reactions were inhibited by the absence of any of the four reagents. Different extraction conditions are shown in the two columns. The left column shows crude lysis, and the right column shows a standard commercial extraction method.

TABLE 25

| Species | SEQ ID NO | Sequence |
|---|---|---|
| Guide nucleic acid | SEQ ID NO: | UAAUUUCUACUAAGUGUAGAUAGCAUUAAGU |
| | 256 | GUCAAGUUCU |
| Reporter nucleic | SEQ ID NO: 9 | /5Alex594N/TTATTATT/3IAbRQSp/ |
| acid | | |
| Target nucleic acid | SEQ ID NO: | TAACTCTGAAAACATTTCTAGTCTTGTAATCAAC |
| | 376 | ATCAGGGTAAAAATCATGTGTTAATACAAAGGT |
| | | ACAGGAACAAAGAATTTGTTCTTCATGGCTCTC |
| | | TGTGTCTGATCCAAGAGGCGAGGCCAGTTTCAT |
| | | TTGAGCATTAAGTGTCAAGTTCTGCACGCTATC |
| | | ATCATCAGGGGCCGAGGCTTCTCTTTGTTTTTAA |
| | | TTAATTGTTTTTAACTGTGAGTTTATATACACTT |
| | | GAAGCA |

FIG. 153 shows the results of dual LAMP-DETECTR assays targeting a flu nucleic acid. Panels in the first and third columns show negative results for LAMP reactions lacking an activator. Samples were detected with a gRNA corresponding to SEQ ID NO: 377 (UAAUUUCUACUAAGUGUAGAUAGCUGCUCGAAUUGGCUUUG R1463) targeted to a target sequence corresponding to SEQ ID NO: 378 (AGCAGAAGCAGAGGATTTGTTTAGTCACTGGCAAACAGGAAAAAAAAATGGCGGA CAACAACATGACCACAACACAAATTGAGGTGGGTCCGGGAGCAACCAATGCCACCA TAAACTTTGAAGCAGGAATTCTGGAGTGCTATGAAAGGCTTTCATGGCAAAGGGCC CTTGACTACCCTGGTCAAGACCGCCTAAACAGACTAAAGAGAAAATTAGAGTCAAG AATAAAGACTCACAACAAAAGTGAGCCTGAAAGTAAAAGGATGTCTCTTGAAGAGA GAAAAGCAATTGGAGTAAAAATGATGAAAGTACTCCTATTTATGAATCCGTCTGCTG GAATTGAAGGGTTTGAGCCATACT). Panels in the second and fourth column show results for LAMP reactions performed in buffer (panel in second column) and viral lysis buffer (panel in fourth column) in the presence of an activator.

### Example 65

### Multi-Chamber Injection-Molded Cartridge for Parallel Amplification and CRISPR Reactions

This example describes a fully integrated device capable of performing multiple amplification and DETECTR reactions on one input sample. The device contains an inlet port for inserting a sample, an injection-molded cartridge containing reagents for the amplification and DETECTR reactions, a fluidic system for partitioning a sample for multiple reactions, detection components for analyzing the reactions, and hardware for processing the reactions. Inserting a sample into the inlet port seals the sample within the device, preventing the sample and surrounding environment from contamination.

FIG. 154 panel (a) shows an injection-molded cartridge. The injection-molded cartridge contains an inlet port 101 for inserting a sample. The bottom of the inlet port is narrow, allowing a swab to snap and seal into place upon insertion. The top of the inlet port is attached to a cap 102 that is configured to hermetically seal the inlet port. The injection-molded cartridge contains fluidic channels 103 (e.g., microfluidic channels) through which samples and reagents can flow, including a metering channel 103a that apportions portions of the sample with defined volumes. The channels are interconnected by locations that can accommodate pumps (e.g., peristaltic pumps, hydraulic pumps, ports connecting to pneumatic pump manifolds, etc.) and switchable vales 104 that direct and meter the fluid flow. Some channels contain or terminate in compartments for reactions 105. The cartridge contains an array of reagent storage compartments 106 coupled to ports 107 for transporting the reagents throughout the fluidic channels and reaction compartments. The injection-molded cartridge is constructed from two pieces 108 & 109 that connect to hermetically seal reagents stored within the cartridge. The injection-molded cartridge chambers further comprise laser bonded sealing layers.

FIG. 154 panel (b) shows a device capable of housing the injection-molded cartridge. The device contains top 110 and bottom 111 platforms designed to hold the injection-molded cartridge firmly in place. The device contains an array of pumps and switchable valves 112 that control hydraulics within the injection-molded cartridge, and heating elements 113 that modulate temperature within the injection-molded cartridge. A fluorimeter 114 housed within the device is capable of measuring fluorescence from detection chambers in the injection-molded cartridge. A computing device 115 controls the fluorimeter, motors, and heating elements within the device.

FIG. 155 shows an assay method utilizing the device that minimizes user input. The method includes off-chip preparation steps that require user input and on-chip automated processes that are controlled by the device. The injection-molded cartridge contains multiple compartments for reagents. Prior to use in an assay, compartments need to be filled with lysis buffer, amplification reagents, and DETECTR reagents including a fluorescence-based reporter, a programmable nuclease, and a guide nucleic acid. The injection-molded cartridge has multiple compartments capable of storing multiple, different sets of amplification and DETECTR reagents (e.g., amplification and DETECTR reagents with different target sequences). Prior to loading, the programmable nuclease and guide nucleic acid need to be incubated at 37° C. for 30 minutes. Once the reagents have been loaded, the injection-molded cartridge can be hermetically sealed, and then loaded into the device. The injection-molded cartridge may be reloadable, or may come pre-loaded with reagents. In such a case, the device can mix and preheat the guide nucleic acid and programmable nuclease prior to performing the DETECTR reaction.

The injection-molded cartridge contains an inlet port for sample insertion. Once the injection-molded cartridge has been prepared with reagents and sealed, a sample can be collected on a swab and inserted into the inlet port. The inlet port is configured so that a swab can be snapped at a break point within the inlet port to fix the sample within the injection-molded cartridge. Once a sample has been fixed in the injection-molded cartridge, the inlet port can be sealed with a hermetic lid.

The sealed injection-molded cartridge (loaded with reagents and a sample) can be inserted into the device, which automates sample preparation and analysis. The device first incubates the sample with 200 μl lysis buffer for 2 minutes. The device meters 20 μl aliquots of the sample into 80 or 180 μl LAMP mastermix for isothermal amplification at 60° C. for 10-60 minutes. 10 μl aliquots of the resulting amplicon are metered into 90 or 190 μl solutions containing DETECTR reagents, and incubated at 37° C. concurrent with real-time excitation and detection at 470 nm and 520 nm. The device collects and transfers this data (e.g., as a radio signal) to computing devices for analysis. The device can perform and detect a large number of sequential and parallel amplification and detection reactions targeting different nucleic acid sequences on a single sample.

FIG. 156 shows optical assemblies for the device. FIG. 156 panel (a) shows an array of diodes 116 that can produce 470 nm light and detect 520 nm or 594 nm light to excite and detect reporter molecules, respectively. FIG. 156 panel (b) shows the diode array with the amber and blue LEDs illuminated. FIG. 156 panel (c) shows an injection molded cartridge illuminated by the diode array.

FIG. 157 shows a possible design for an injection-molded cartridge. The injection-molded cartridge contains a sample chamber 117 for collecting and then mixing a sample with up to 400 μl of buffer. The sample chamber contains a pump, and is connected through a rotary valve to a series of fluidic channels 118 (e.g., microfluidic channels) which partition the sample into multiple amplification chambers 119. A metering valve within the rotary valve at the exit of the sample chamber dispenses 20 μl aliquots from the sample chamber via into the fluidic channels per rotation. The amplification chambers are coupled to amplification reagent chambers (which contain reagents for the amplification reactions) 120 through resistance channels 118b, which are each configured with a pump and a valve that control the flow of stored reagents into the amplification chambers. The back end of each amplification chamber is connected to a valve that meters flow through a second series of fluidic channels 121 into a series of detection chambers 122. The detection chambers are coupled to detection reagent chambers (which store reagents for the detection reactions) 123 through resistance channels 118b, which are each configured with a pump and a valve that control the flow of stored reagents into the detection chambers. This injection-molded cartridge contains one sample chamber, 5 amplification chambers, and 10 detection chambers.

### Example 66

### Injection-Molded Cartridge Design for Performing Multiple Amplification and DETECTR Reactions on a Single Sample

This example provides a design for an injection molded cartridge capable of partitioning a sample for separate amplification and DETECTR reactions. The injection-molded cartridge is designed to collect samples from swabs (e.g., buccal swabs). The combinations of distinct amplification and DETECTR reactions allow the sample to be assayed for multiple sequences. For example, the 8 DETECTR reaction could be used to query for 8 separate viruses or 7 viruses and an internal control. The injection-molded cartridge is designed to fit within a device that automates sample and reagent movement, heating, and detection.

FIG. 158 shows an injection-molded cartridge design with 1 sample chamber 124, 4 amplification chambers 125, and 8 detection chambers 126. Each amplification chamber and detection chamber is connected by a resistance channel 129b to one amplification reagent chamber 127 or one detection reagent chamber 128, respectively. Each series of chambers is connected by fluidic channels 129 as shown in FIG. 158. The fluidic channels connecting the sample chamber to the amplification chambers are between 300 μm and 1 mm in width.

FIG. 159 shows an alternate design for the injection-molded cartridge in FIG. 158, with an lysis reagent chamber 130 connected to the sample chamber 124. A valve (v1) mediates flow between the lysis reagent chamber and sample chamber. V1-V18 correspond to valves to control flow between chambers.

FIG. 160 shows a design for the top of an injection-molded cartridge similar to the one depicted in FIG. 159. The injection-molded cartridge can be connected to a manifold for pressure-driven flow. The labeled chambers C1 and C2 correspond to the lysis reagent chamber and sample chamber in FIG. 159. The labeled chambers C3-C6 correspond to the amplification reagent chambers in FIG. 159. The labeled chambers C7-C10 correspond to the amplification chambers in FIG. 159. The labeled chambers C11-C18 correspond to the detection reagent chambers in FIG. 159. The labeled chambers C19-C26 correspond to the detection chambers in FIG. 159. In this design, the sample chamber and the lysis reagent chamber are located near the center of the injection-molded cartridge. The valves controlling flow from C3-C6 and C11-C18 can be controlled 131 from the top of the injection molded cartridge. The detection reagent chambers and detection chambers are also spaced further from the amplification chambers to further isolate detection reagents (e.g., reagents for CRISPR reactions) from the temperatures of the amplification reactions, as in some cases, detection reagents (e.g., CRISPR reaction reagents) aren't stable at the temperatures required for amplification reactions.

FIG. 161 shows a design for a portion of an injected molded cartridge containing a sample chamber 132 and a lysis reagent chamber 133 that are connected by a rotary valve 134, which is sealed with laser bonded clear polycarbonate. A swab containing a sample can be inserted into the sample chamber. Lysis buffer can be pumped from the lysis reagent chamber to the sample chamber by a partial rotation of a rotary valve 134. The rotary valve contains a metering channel 135a that can transfer a defined volume of liquid from the sample compartment into a channel 135b leading to an amplification chamber 136. Thus, the device is capable of sequentially transfer aliquots from the sample chamber to each of the individual amplification chambers. Flow out of each amplification chamber is controlled by a valve 137, which is connected to a vent. Panel A shows the rotary valve connecting the lysis reagent chamber to the sample chamber. Panel B shows the injection-molded cartridge after the rotary valve has been partially rotated (relative to panel A).

FIG. 162 shows a design for a portion of an injected molded cartridge containing an amplification reagent chamber 138 and an amplification chamber 139 connected by a slider valve 140. The slider valve has four positions, a first position for delivering fluid into the amplification chambers (shown in panel A) through a first metering channel 141, two positions for metering fluid out of the amplification chamber and into metering channels 142 & 143 (one of these two positions is depicted in panel B), and a fourth position in which the metering channels connect to fluidic channels 144 & 145 leading to separate detection chambers (shown in panel C). A valve 146 in between the amplification reagent chamber and amplification chamber controls flow between the two chambers when the slider valve is in the first of the four positions (shown in panel A).

FIG. 163 shows a design for an injection-molded cartridge with a plastic shell. The design includes a sample inlet port 147 leading to the sample chamber with a hermetically sealing cap 148. The sample inlet port is designed to accommodate a swab 149. Lysis buffer can be loaded into the top of the sample inlet port prior to insertion of the swab. Insertion of the swab breaks a seal, allowing the lysis buffer to flow through the bottom of the sample inlet port and into the sample chamber. Once inserted, the swab locks in place against a set of plastic projections 150, minimizing sample contamination. Closing the cap over the sample inlet port further protects against contamination. The design is rectangular so that the detection chambers 151 have flat faces for excitation light to pass through during fluorescence detection. The slider valve 152 that meters flow through the amplification chambers can be seen near the back of the injection-molded cartridge. The top of the injection-molded cartridge contains multiple ports 153 terminating in O-rings 154 allow the cartridge to connect to a pneumatic pumping manifold that can apply pressure to individual cartridge chambers. Panel A depicts a design for an injection-molded cartridge. Panel B is a picture of a functional model of an injection-molded cartridge similar to the one shown in panel A. The injection-molded cartridge in panel C differs from the injection molded cartridges in panel A by its sample inlet port, which lacks the breakable seal and projections for holding a swab.

FIG. 164 panel A shows a bottom view of an injection-molded cartridge design. This design features wide, flat reagent chambers (e.g., amplification reagent chambers) to enable rapid heating and fast fluid mixing by pumping the fluids back and forth into and out of reagent chambers, rather than sequentially flowing different solutions into a single chamber. The short cartridge height allows a heater to wrap around the reaction compartments. The lengths of the channels 155 that connect the same types of chambers to provide equal fluidic resistance when used for mixing. The bottom of the sliding valve 156, amplification reagent chambers 157 and detection chambers 158 can be seen from the bottom of the cartridge. Panel B shows a top view of the injection-molded chip. Top 159 and bottom 160 plastic casing pieces form a hermetic seal around the injection molded chip. Interlocking clips 161 on the plastic casing pieces facilitate easy assembly into a single unit. A series of 0-ring topped ports 162 allow the injection molded cartridge to couple to a pneumatic pumping manifold that can control flow throughout the injection-molded cartridge. A sample inlet port 163 contains a top chamber stoppered by a breakable seal 164.

### Example 67

### Injection-Molded Cartridge Capable of Performing Parallel Amplification and CRISPR Reactions on a Single Sample

This example describes an injection-molded cartridge designed to perform multiple amplification and CRISPR reactions on a single sample. This cartridge has 4 amplification chambers and 8 detection chambers. A single sample will first be diluted in a sample chamber, and then be partitioned between the four amplification chambers. The amplification products from each amplification chamber will be partitioned to two separate detection chambers. Each amplification chamber is transparent so as to allow optical (e.g., fluorescent) monitoring of the CRISPR (e.g., DETECTR) reactions. Each amplification and detection chamber is connected to a unique reagent storage chamber (e.g., an amplification reagent chamber). Some chambers can be loaded with identical reagents, or each chamber can be loaded with different reagents (e.g., amplification reagents and DETECTR reagents targeting different sequences). Thus, the injection-molded cartridge is capable of performing up to 8 unique sequences of amplification and CRISPR reactions on a single input sample.

The injection-molded cartridge is configured to insert into a device capable of controlling sample partition, reagent loading, heating and detection within the cartridge. The cartridge contains multiple valves along with a pneumatic delivery manifold, which collectively allow a device to control the flow, pressure, and temperature in the chambers and fluidic channels within the device. The device can also be equipped with an optical detector (e.g., a fluorimeter) capable of measuring the components of the detection chambers.

FIG. 165 shows designs for a portion of the injection-molded cartridge containing the sample chamber 101 and amplification chambers 102. Panels A & B provide top-down views, while panels C through E show the injection-molded cartridge from the bottom. As shown in panel A, a swab 103 containing the sample to be analyzed can be inserted into a sample inlet port 104. The sample inlet port has a hermetically sealing cap 105, which seals the contents of the injection-molded cartridge from the surrounding environment. Once a sample has been inserted into the sample chamber, a rotating valve 106 can transport lysis buffer from a lysis buffer storage chamber 107 to the sample chamber. Panel A shows the rotating valve connecting the lysis buffer storage and sample chambers. Once sample lysis has completed, the rotating valve can transfer 20 μl aliquots of the sample into a metering channel 108 that can be rotated to deliver sample into microfluidic channels 109 leading to the four amplification reagent chambers 110. Panel B shows the rotating valve positioned to connect the metering channel with the sample chamber.

As shown from the bottom-up view depicted in panel C, the contents of the amplification reagent chambers can flow into the amplification chambers 101. Mixing is performed by moving the contents back and forth between the two chambers. Once mixing is complete, the samples are completely transferred into the amplification chambers and incubated for a controlled period of time. As is shown in panel D, the inj ection-molded cartridge can be situated over a heating element within the control-device, thus allowing temperature control during the amplification during.

The direction of flow into and out of the amplification chambers is mediated by a slider valve 111. Panel C depicts the slider valve in a first position that connects each amplification reagent chamber to an amplification chamber. Once the amplification reaction is complete, the panel can slide to second and third positions (one of which is depicted in panel E) that allow sample to move from the amplification chambers into metering channels 112. The slider is then capable of adopting a fourth position in which the metering channels overlap with channels 113 that lead to the detection reagent chambers. Thus, the sample is divided into 8 separate components following amplification.

FIG. 166 panel A provides a design for the portion of the injection-molded cartridge containing the detection reagent chambers and detection chambers. Following amplification, the sample flows from the amplification chambers and into the detection reagent chambers 114. The sample then flows from the detection reagent chambers and cascades downwards into the detection chambers 115. The injection-molded cartridge connects to a plastic cover piece, which fits over the top of the cartridge and seals its chambers. Panel B shows the injection-molded cartridge with the plastic cover piece 116. As is shown in the side-on view of panel B, the detection chambers have flat, transparent surfaces enabling fluorescence excitation and detection. The detection chambers are situated over a second heater in the control device capable of elevating the temperatures of the detection chambers. Black bosses between the detection chambers minimizes light contamination between chambers, thus improving the accuracy and sensitivity of optical experiments (e.g., luminescence detection, fluorescence, etc.).

FIG. 167 panels A and B provide full views of the injection molded cartridge. The amplification chambers 102, lysis buffer storage chamber 107, amplification reagent chambers 110, and detection reagent chambers 114 are open, and can be loaded with solutions and reagents. Once desired reagents are loaded into the device, a plastic cover piece can be attached to the injection-molded cartridge, sealing the chambers and fluidic channels within the device. Panel C shows a picture of a working physical model of the injection molded cartridge with the plastic cover piece 116 attached. The plastic cover piece contains an array of O-ring topped inlet ports 118 that can connect to a pneumatic manifold capable of directing flow throughout the chambers and fluidic channels within the injection-molded cartridge. The total dimensions of the cartridge are 92 mm x 80 mm x 52.5 mm including the height of the sample inlet port, and 92 mm×80 mm×19.5 mm excluding the sample inlet port. A retaining ring forms a seal between the injection-molded cartridge and inlet port, which are otherwise distinct and separable.

### Example 68

### Diode Array for Excitation and Detection of Fluorescent Detection from an Injection-Molded Cartridge

This example covers a detection scheme for fluorescent read-out DETECTR reactions in a multi-chamber cartridge. The cartridge is designed to perform separate DETECTR reactions on separate portions of a sample that have undergone amplification. FIG. 168 shows an injection-molded cartridge 101 housed in a device 102 containing a diode array capable of detecting light from each of the chambers and white light emitting diodes 103 positioned to illuminate the chambers. The injection-molded cartridge has 8 detection chambers 104. The four leftmost (orange) detection chambers contain the dye ATTO 594, and the four rightmost chambers contain the dye ATTO 488. The front faces (pointing out of the device opening) of the detection chambers that contain the 594 dye are coated with an orange gel filter. The front faces of the detection chambers that contain the 488 dye are coated with a yellow filter. White lights illuminate the detection chambers from the side, exciting fluorescent dyes within the detection chambers. The sides of the detection chambers facing the white lights may be coated with optical filters or color-absorbent gels. The device contains diodes that detect light emitted from the detection chambers, thus allowing the device to monitor DETECTR reactions with fluorescent reporters.

FIG. 169 panels A and B show a graphic user interface for controlling the white lights, detector diodes, and for monitoring data collected on the detector diodes. The graphic user interface allows the user to set temperature shutoff points (e.g., configure a detector diode to shut off if its temperature exceeds 50° C.), the bias voltage or current through the diodes, and the sampling rate (e.g., 100 Hz) on each detector diode. The graphic can display fluorescence readout data from each detector diode.

FIG. 170 shows the results of a calibration test for the diode array. Each set of 8 datapoints corresponds to the data collected by the 8 detector diodes in a single test. Data set A was collected without an injection molded cartridge in the device. Data sets B-H were collected with an empty injection molded cartridge in the device. Data set B was collected on the empty cartridge. Data sets C and D were collected with the cartridge containing buffer but no dye. Data sets E, F and G were collected with the cartridge containing 1 nM, 10 nM and 100 nM dye, with diodes 1-4 collecting on wells containing ATTO 488 and wells 5-8 containing ATTO 594. Data set H was collected with 100 nM ATTO 488 in wells 1-3, 1 uM ATTO 488 in well 4, 100 nM ATTO 594 in wells 5-7, and 1 μM ATTO 594 in well 8. FIG. 170 shows bar graphs in 8 sections designated as A, B, C, D, E, F, G, and H. Section 1 is LEDS on, no chip, Section B is LEDS on, empty chip, Section C is LEDS on, chip with 100 ul 1× TE, Section D is LEDS on, chip with 90 ul 1× TE, Section E is 90 ul of 1 nM dye, Section F is 90 ul of 10 nM dye, Section G is 90 ul of 100 nM dye, and Section H is 100 nM and 1 uM. Within each section are 7 bars, which from left to right are DIODE 1, DIODE 2, DIODE 3, DIODE 4, DIODE 5, DIODE 6, DIODE 7, and DIODE 8. The y-axis shows fluorescence in arbitrary units (a.u.) ranging from 2.4 to 3.0 in increments of 0.1.

### Example 69

### HERC2 DETECTR Assay Performed Measured with a Diode Array

This example describes a DETECTR Assay performed on the injection molded cartridge of EXAMPLE 67 using the diode array of EXAMPLE 68. The reagents for the DETECTR assays were loaded directly into the detection chambers. The assays utilized a programmable nuclease with SEQ ID NO: 37, a guide nucleic acid with SEQ ID NO: 256 targeting HERC2 G SNP allele, and a reporter nucleic acid which increased fluorescence response upon cleavage. Four wells contained 5 μM reporter, 150 nM programmable nuclease, 600 nM guide nucleic acid, and 500 pM target nucleic acid. Two wells contained 5 μM reporter, 150 nM programmable nucleic acid, 600 nM guide nucleic, and no target. Two wells contained only buffer. The reporters contained either ATTO 488 or ATTO 594.

FIG. 171 shows fluorescence traces from the 8 detection chambers measured by an 8 diode detector array. The detection chambers containing a reporter, programmable nuclease, guide nucleic acid, and target nucleic acid provided fluorescence responses that increased linearly with time. The detection chambers containing DETECTR reagents but lacking the target nucleic acid and the detection chambers containing only buffer did not display increases in fluorescence. Thus, the detection chambers with active transcollateral reporter cleavage were distinguishable by fluorescence. FIG. 171 shows line graphs with the x-axis showing the DETECTR timepoint in minutes ranging from 0 to 35 in increments of 5 and the y-axis showing the net fluorescence in arbitrary units (au.) ranging from −0.02 to 0.12 in increments of 0.02. The four lines linearly increasing are from left/highest to right/lowest are G-SNP—488 nm, G-SNP—594 nm, G-SNP—488 nm, and G-SNP—488 nm. The last two in the prior list are nearly overlapping. The higher flat line near the bottom corresponds to DETECTR MM—488 nm. The lower flat lines at the bottom correspond to DETECTR MM—594 nm and 1× TE—594 nm.

FIG. 172 shows an image of the detection chambers 30 minutes after DETECTR reagent addition. Detection chambers 1, 4, 5, and 8 contained the target nucleic acid, and are visibly brighter than the remaining detection chambers.

### Example 70

### Amplification of a Target Nucleic Acid in a Viral Lysis Buffer

This example describes amplification of a target nucleic acid in a viral lysis buffer. The effects of various buffer compositions, reducing agents, and incubation temperatures were tested on amplification of a target nucleic acid. Samples in different buffers were amplified using LAMP amplification, and the resulting fluorescence was measured. Higher fluorescence was indicative of more amplification.

FIG. 173 shows the results of amplification of a SeraCare target nucleic acid using LAMP under different lysis conditions. Samples were amplified in various buffers. Samples were incubated for 5 minutes at either room temperature (left plots) or 95° C. (right plots). Samples containing either no target (“NTC”), 2.5, 25, or 250 copies per reaction. Assays were performed in triplicate using 5 μL of sample in a 25 μL reaction.

FIG. 174 shows the results of amplification of a SeraCare standard target nucleic acid using LAMP under different lysis conditions. Samples were amplified in various buffers. Samples containing either no target (“NTC”), 1.5, 2.5, 15, 25, 150, or 250 copies per reaction. Assays were performed in triplicate using 3 μL of sample in a 15 μL reaction or 5 μL of sample in a 25 μL reaction.

The results of this experiment demonstrated certain buffers were more conducive to LAMP amplification.

### Example 71

### Amplification of a Target Nucleic Acid from COVID-19 Patient Samples in a Viral Lysis Buffer

This example describes amplification of a target nucleic acid from COVID-19 patient samples in a viral lysis buffer. Samples collected from patients positive for COVID-19 were lysed and amplified in viral lysis buffers with varying components. Target nucleic acids corresponding to the SARS-CoV-2 N gene and RNaseP were amplified using LAMP as described in EXAMPLE 22. Various viral lysis buffer formulations were tested.

FIG. 175 shows amplification of a SARS-CoV-2 N gene (“N”) and an RNase P sample input control nucleic acid (“RP”) in the presence of six different viral lysis buffers (“VLB,” “VLB-D,” “VLB-T,” “Buffer,” “Buffer-A,” and “Buffer-B”). Buffer-A contains Buffer with Reducing Agent A and Buffer-B contains Buffer with Reducing Agent B. Shaded squares indicate rate of amplification, with darker shading indicating faster amplification. Amplification was performed at either 95° C. (“95 C”) or room temperature (“RT”) on high, medium, or low titer COVID-19 positive patient samples (“16.9,” “30.5,” and “33.6,” respectively). Samples were measured in duplicate.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A microfluidic cartridge for detecting a target nucleic acid comprising:
a) an amplification chamber fluidically connected to a valve;
b) a detection chamber fluidically connected to the valve, wherein the valve is connected to a sample metering channel;
c) a detection reagent chamber fluidically connected to the detection chamber via a resistance channel, the detection reagent chamber comprising a programmable nuclease, a guide nucleic acid, and a labeled detector nucleic acid, wherein the labeled detector nucleic acid is capable of being cleaved upon binding of the guide nucleic acid to a segment of a target nucleic acid.

2. The microfluidic cartridge of claim 1, wherein the sample metering channel (i) controls volumes of liquids dispensed in a channel or chamber, and (ii) is fluidically connected to the detection chamber.

3. (canceled)

4. The microfluidic cartridge of claim 1, wherein the resistance channel (i) has a serpentine path, an angular path, or a circuitous path, and (ii) is fluidically connected with the valve.

5. The microfluidic cartridge of claim 1, wherein the valve:
(a) is a rotary valve, pneumatic valve, a hydraulic valve, an elastomeric valve;
(b) comprises casing, comprising a substrate or an oxer-mold;
(c) is actuated by a solenoid;
(d) is controlled manually, macmetically, electrically, thermally, by a bistable circuit, with a piezoelectric material, electrochemically, with phase change, theologically, pneumatically, with a check valve, with capillarity, or any combination thereof; or
(e) is fluidically connected to two or more detection chambers via an amplification mix sputter.

6.-9. (canceled)

10. The microfluidic cartridge of claim 1, wherein the valve is a rotary valve that fluidically connects at least 3, at least, 4, or at least 5 chambers.

11. The microfluidic cartridge of claim 1, further comprising one or more of:
(a) an amplification reagent chamber fluidically connected to the amplification chamber;
(b) a sample chamber fluidically connected to the amplification reagent chamber;
(c) a sample inlet connected to the sample chamber;
(d) a lysis buffer storage chamber connected to the sample chamber;
(e) a second valve fluidically connecting the lysis buffer storage chamber to the sample chamber; or
(f) a second valve fluidically connected to the detection reagent chamber and the detection chamber.

12.-20. (canceled)

21. The microfluidic cartridge of claim 11, wherein
(a) the sample chamber is fluidically connected to the amplification chamber through the amplification reagent chamber;
(b) the sample chamber is fluidically connected to the amplification reagent chamber through the amplification chamber; or
(c) the microfluidic cartridge is configured to direct fluid bidirectionally between the amplification reagent chamber and amplification chamber.

22.-23. (canceled)

24. The microfluidic cartridge of claim 1, wherein:
(a) the detection reagent chamber is fluidically connected to the amplification chamber;
(b) the amplification chamber is fluidically connected to the detection chamber through the detection reagent chamber;
(c) the microfluidic cartridge further comprises a reagent port above the detection chamber configured to deliver fluid from the detection reagent chamber to the detection chamber;
(d) the ampification chamber is fluidically connected to the detection reagent chamber through the detection chamber;
(e) the amplification chamber and detection chamber are thermally isolated;
(f) the detection reagent chamber is fluidically connected to the detection chamber; and/or
(g) the detection reagent chamber is fluidically connected to the detection chamber via a second resistance channel.

25.-27. (canceled)

28. The microfluidic cartridge claim 1, wherein the resistance channel is configured to reduce backflow into the detection chamber and the detection reagent chamber.

29. The microfluidic cartridge of claim 2, wherein the sample metering channel is configured to direct a predetermined volume of fluid from the detection reagent chamber to the detection chamber.

30.-37. (canceled)

38. The microfluidic cartridge of claim 1, wherein:
(a) the microfluidic cartridge is configured to connect to a first pump to pump fluid from the amplification chamber to the detection chamber;
(b) the microfluidic cartridge is configured to connect to a second pump to pump fluid from the detection reagent chamber to the detection chambers;
(c) the amplification chamber is fluidically connected to a port configured to receive pneumatic pressure; and/or
(d) the microfluidic cartridge comprises 5-7 layers.

39.-42. (canceled)

43. The microfluidic cartridge of claim 11, wherein:
(a) the amplification reagent chamber is connected to a second port configured to receive pneumatic pressure; or
(b) the microfluidic cartridge is configured to connect to a third pump to pump fluid from the amplification reagent chamber to the amplification chamber.

44.-46. (canceled)

47. The microfluidic cartridge of claim 1, wherein:
(a) the detection reagent chamber is connected to a port configured to receive pneumatic pressures;
(b) the detection reaaern chamber is fluidically connected to a third port through a third channel;
(c) the microfluidic cartridge is configured to connect to a fourth pump to pump fluid from the detection reagent chamber to the detection chamber; and/or
(d) the microfluidic cartridge further comprises a plurality of ports configured to couple to a gas manifold, wherein the plurality of ports is configured to receive pneumatic pressure.

48.-56. (canceled)

57. The microfluidic cartridge of claim 2, wherein:
(a) a region of the resistance channel is molded to direct flow in a direction perpendicular to the net flow direction;
(b) a region of the resistance channel is molded to direct flow in a direction perpendicular to the axis defined by two ends of the resistance channel; or
(c) a region of the resistance channel is molded to direct flow along the z-axis of the microfluidic cartridge.

58.-62. (canceled)

63. The microfluidic cartridge of claim 1, wherein the detection chamber: (a) is vented with a hydrophobic PTFE vent, and/or (b) comprises an optically transparent surface.

64. (canceled)

65. The microfluidic cartridge of claim 1, wherein:
(a) the amplification chamber is configured to hold from 10 μL to 500 μL of fluid;
(b) the microfluidic cartridge is configured to accept from 2 μL to 100 μL, of a sample comprising a nucleic acid;
(c) the amplification reagent chamber comprises between 5 and 200 μl an amplification buffer;
(d) the detection reagent chamber stores from 5 to 200 μl of fluid containing the programmable nuclease, the guide nucleic acid, and the labeled detector nucleic acid; and/or
(e) the detection chamber holds up to 100 μL, 200 μL, 300 μL, or 400 μL of fluid.

66.-70. (canceled)

71. The microfluidic cartridge of claim 1, comprising 2, 3, 4, 5, 6, 7, or 8 detection chambers, optionally wherein the detection chambers are fluidically connected to a single sample chamber.

72.-78. (canceled)

79. The microfluidic cartridge of claim 1, further comprising a sliding valve; optionally wherein the sliding valve:
(a) connects the amplification reagent chamber to the amplification chamber;
(b) connects the amplification chamber to the detection reagent chamber; and/or
(c) connects the amplification reagent chamber to the detection chamber.

80.-83. (canceled)

84. A manifold configured to accept the microfluidic cartridge of claim 1, the manifold comprising a pump configured to pump fluid into the detection chamber, an illumination source configured to illuminate the detection chamber, a detector configured to detect a detectable signal produced by the labeled detector nucleic acid, and a heater configured to heat the amplification chamber.

85. The manifold of claim 84, wherein:
(a) the manifold further comprises a second heater configured to heat the detection chamber;
(b) the illumination source is a broad spectrum light source;
(c) the illumination source light produces an illumination with a bandwidth of less than 5 nm;
(d) the illumination source is a light emitting diode;
(e) the detectable signal is light;
(f) the detector is a camera or a photodiode
(g) the detector has a detection bandwidth of less than 100 nm, less than 75 nm, less than 50 nm, less than 40 nm, less than 30 nm, less than 20 nm, less than 10 nm, or less than 5 nm;
(h) the manifold further comprises an optical filter configured to be between the detection chamber and the detector.

86.-93. (canceled)

94. The microfluidic cartridge of claim 1, wherein the amplification chamber comprises amplification reagents and/or lysis buffer.

95.-108. (canceled)

109. The microfluidic cartridge of claim 1, wherein the programmable nuclease:
(a) comprises an RuvC catalytic domain:
(b) is a type V CRISPR/Cas effector protein;
(c) is a Cas12 protein;
(d) comprises a Cas12a polypeptide, a Cas12b polypeptide, a Cas12c polypeptide, a Cas12d polypeptide, a Cas12e polypeptide, a C2c4 polypeptide, a C2c8 polypeptide, a C2c5 polypeptide, a C2c10 polypeptide, and a C2c9 polypeptide;
(e) has at least 80%, at least 85%, at least 90%, at least 92%, at least 95%, at least 97%, or at least 99% sequence identity to any one of SEQ ID NO: 27-SEQ ID NO: 37;
(f) is a Cas14 protein;
(g) comprises a Cas14a polypeptide, a Cas14b polypepetide, a Cas14c polypeptide, a Cas14d polypeptide, a Cas14e polypeptide, a Cas14f polypeptide, a Cas14g polypeptide, a Cas14h polypeptide, a Cas14i polypeptide, a Cas14j polypeptide, or a Cas14k polypeptide;
(h) has at least 80%, at least 85%, at least 90%, at least 92%, at least 95%, at least 97%, or at least 99% sequence identity to any one of SEQ ID NO: 38-SEQ ID NO: 129;
(i) is a CasΦ protein; or
(j) has at least 80%, at least 85%, at least 90%, at least 92%, at least 95%, at least 97%, or at least 99% sequence identity to any one of SEQ ID NO: 274-SEQ ID NO: 321.

110.-121. (canceled)

122. The microfluidic cartridge of claim 1, the microfluidic cartridge further comprising (i) one or more chambers for in vitro transcribing amplified coronavirus target nucleic acid, and (ii) reagents for in vitro transcription.

123.-124. (canceled)

125. The microfluidic cartridge of claim 1, wherein the programable nuclease comprises a HEPN cleaving domain, a type VI CRISPR/Cas effector protein, or a Cas13 protein.

126.-141. (canceled)

142. The microfluidic cartridge of claim 1, wherein the microfluidic cartridge comprises a control nucleic acid.

143. The microfluidic cartridge of claim 142, wherein:
(a) the control nucleic acid is in the detection chamber;
(b) the control nucleic acid is RNaseP;
(c) the control nucleic acid has a sequence of SEQ ID NO: 379; or
(d) the guide nucleic acid has at least 80%, at least 85%, at least 90%, at least 92%, at least 95%, at least 97%, or at least 99% sequence identify to any one of SEQ ID NO: 330-SEQ ID NO: 332.

144.-148. (canceled)

149. The microfluidic cartridge of claim 1, wherein:
(a) the guide nucleic acid targets a plurality of target sequences;
(b) the microfluidic cartridge comprises a plurality of guide sequences tiled against a virus;
(c) the labeled detector nucleic acid comprises a single stranded reporter comprising a detection moiety;
(d) the labeled detector moduces a detectable signal upon cleavage of the detector nucleic acid.

150.-156. (canceled)

157. A method of detecting a target nucleic acid, the method comprising:
a) providing a sample from a subject;
b) adding the sample to the microfluidic cartridge of claim 1;
c) detecting a detectable signal produced by a cleavage product of the labeled detector nucleic acid in response to the presence of the target nucleic acid; and
d) optionally quantifying the detectable signal, thereby quantifying an amount of the target nucleic acid present in the sample.

158.-164. (canceled)

165. A composition comprising a non-naturally occurring nucleic acid comprising a sequence according to any one of SEQ ID NOs: 348-353.

166. A composition comprising a non-naturally occurring nucleic acid comprising a sequence according to any one of SEQ ID NOs: 354-359.
